(19) United States
(12) Patent Application Publication
(10) Pub. No.: US 2023/0012687 A1
(43) Pub. Date: Jan. 19, 2023

(54) Polynucleotides, Compositions, and Methods for Polypeptide Expression

(71) Applicant: Intellia Therapeutics, Inc., Cambridge, MA (US)

(72) Inventors: Bradley Andrew Murray, Saratoga, CA (US); Christian Dombrowski, Auburndale, MA (US); Seth C. Alexander, Medford, MA (US)

(73) Assignee: Intellia Therapeutics, Inc., Cambridge, MA (US)

(21) Appl. No.: 17/486,039

(22) Filed: Sep. 27, 2021

Related U.S. Application Data
(63) Continuation of application No. PCT/US2020/025372, filed on Mar. 27, 2020.
(60) Provisional application No. 62/825,656, filed on Mar. 28, 2019.

(51) Int. Cl. C12N 15/67; C12N 9/22; C12N 15/90; C12N 15/88; C12N 15/11

(52) U.S. Cl. CPC C12N 15/67; C12N 9/22; C12N 15/907; C12N 2310/20; C12N 15/11; C12N 2800/22; C12N 15/88

(57) ABSTRACT

Compositions and methods for gene editing. In some embodiments, a polynucleotide encoding Cas9 is provided that can provide one or more of improved editing efficiency, reduced immunogenicity, or other benefits.

This patent application is a continuation of International Application No. PCT/US2020/025372, filed on Mar. 27, 2020, which claims priority to U.S. Provisional Application No. 62/825,656, filed Mar. 28, 2019, the content of which are incorporated herein by reference in their entirety for all purposes.

The instant application contains a Sequence Listing which has been submitted electronically in ASCII format and is hereby incorporated by reference in its entirety. Said ASCII copy, created on Sep. 27, 2021, is named 01155-0027-00US_ST25.txt and is 365 KB in size.

The present disclosure relates to polynucleotides, compositions, and methods for polypeptide expression, including expression from mRNAs and expression from expression constructs.

### INTRODUCTION AND SUMMARY

Useful polypeptides can be produced in situ by cells contacted with polynucleotides, such as mRNAs or expression constructs. Existing approaches, e.g., in certain cell types or organisms such as mammals, may, however, provide less robust expression than desired or may be undesirably immunogenic, e.g., may provoke an undesirable elevation in cytokine levels.

Thus, there is a need for improved polynucleotides, compositions, and methods for polypeptide expression. The present disclosure aims to provide compositions and methods for polypeptide expression that provide one or more benefits such as at least one of improved expression levels, increased activity of the encoded polypeptide, or reduced immunogenicity (e.g., reduced elevation in cytokines upon administration), or at least to provide the public with a useful choice. In some embodiments, a polynucleotide encoding a polypeptide is provided, wherein one or more of its coding sequence or codon pair content differs from existing polynucleotides in a manner disclosed herein. It has been found that such features can provide benefits such as those described above. In some embodiments, the improved expression occurs in or is specific to an organ or cell type of a mammal, such as the liver or hepatocytes.

The following embodiments are provided by this disclosure.

Embodiment 1 is a polynucleotide comprising (i) an open reading frame (ORF) encoding a polypeptide, wherein at least 1.03% of the codon pairs in the ORF are codon pairs shown in Table 1; or (ii) an open reading frame (ORF) encoding a polypeptide, wherein at least 1% of the codon pairs in the ORF are codon pairs shown in Table 1 and the ORF does not encode an RNA-guided DNA binding agent.

Embodiment 2 is a polynucleotide comprising an open reading frame (ORF) encoding a polypeptide, wherein the ORF comprises a sequence with at least 95% identity to any one of SEQ ID NOs: 6-10, 29, 46, 69-73, 90-93, 96-99, 102-105, 108-111, 114-117, 120-123, 126-129, or 132-143, optionally wherein identity is determined without regard to the start and stop codons of the ORF.

Embodiment 3 is a polynucleotide comprising an open reading frame (ORF) encoding a polypeptide, wherein at least 75%, 80%, 85%, 90%, 95%, 98%, 99%, or 100% of the codons in the ORF are (i) codons listed in Table 5, or (ii) codons listed in Table 6, and wherein the polypeptide is not an RNA-guided DNA binding agent.

Embodiment 4 is the polynucleotide of any one of embodiments 1-3, wherein the repeat content of the ORF is less than or equal to 23.3%.

Embodiment 5 is the polynucleotide of any one of embodiments 1-4, wherein the GC content of the ORF is greater than or equal to 55%.

Embodiment 6 is a polynucleotide comprising an open reading frame (ORF) encoding a polypeptide, wherein the repeat content of the ORF is less than or equal to 23.3% and the GC content of the ORF is greater than or equal to 55%.

Embodiment 7 is the polynucleotide of any one of embodiments 2-6, wherein at least 1.03% of the codon pairs in the ORF are codon pairs shown in Table 1.

Embodiment 8 is the polynucleotide of any one of embodiments 1-7, wherein less than or equal to 0.9% of the codon pairs in the ORF are codon pairs shown in Table 2.

Embodiment 9 is the polynucleotide of any one of embodiments 1-8, wherein at least 60%, 65%, 70%, or 75% of the codon in the ORF are codon shown in Table 3.

Embodiment 10 is the polynucleotide of any one of embodiments 1-9, wherein less than or equal to 20% of the codons in the ORF are codons shown in Table 4.

Embodiment 11 is the polynucleotide of any one of embodiments 1-10, wherein at least 1.05% of the codon pairs in the ORF are codon pairs shown in Table 1.

Embodiment 12 is the polynucleotide of any one of embodiments 1-10, wherein at least 1.1% of the codon pairs in the ORF are codon pairs shown in Table 1.

Embodiment 13 is the polynucleotide of any one of embodiments 1-10, wherein at least 1.2% of the codon pairs in the ORF are codon pairs shown in Table 1.

Embodiment 14 is the polynucleotide of any one of embodiments 1-10, wherein at least 1.3% of the codon pairs in the ORF are codon pairs shown in Table 1.

Embodiment 15 is the polynucleotide of any one of embodiments 1-10, wherein at least 1.4% of the codon pairs in the ORF are codon pairs shown in Table 1.

Embodiment 16 is the polynucleotide of any one of embodiments 1-10, wherein at least 1.5% of the codon pairs in the ORF are codon pairs shown in Table 1.

Embodiment 17 is the polynucleotide of any one of embodiments 1-10, wherein at least 1.6% of the codon pairs in the ORF are codon pairs shown in Table 1.

Embodiment 18 is the polynucleotide of any one of embodiments 1-10, wherein at least 1.7% of the codon pairs in the ORF are codon pairs shown in Table 1.

Embodiment 19 is the polynucleotide of any one of embodiments 1-10, wherein at least 1.8% of the codon pairs in the ORF are codon pairs shown in Table 1.

Embodiment 20 is the polynucleotide of any one of embodiments 1-10, wherein at least 1.9% of the codon pairs in the ORF are codon pairs shown in Table 1.

Embodiment 21 is the polynucleotide of any one of embodiments 1-10, wherein at least 2.0% of the codon pairs in the ORF are codon pairs shown in Table 1.

Embodiment 22 is the polynucleotide of any one of embodiments 1-10, wherein at least 2.1% of the codon pairs in the ORF are codon pairs shown in Table 1.

Embodiment 23 is the polynucleotide of any one of embodiments 1-10, wherein at least 2.3% of the codon pairs in the ORF are codon pairs shown in Table 1.

Embodiment 24 is the polynucleotide of any one of embodiments 1-10, wherein at least 2.4% of the codon pairs in the ORF are codon pairs shown in Table 1.

Embodiment 25 is the polynucleotide of any one of embodiments 1-10, wherein at least 2.5% of the codon pairs in the ORF are codon pairs shown in Table 1.

Embodiment 26 is the polynucleotide of any one of embodiments 1-10, wherein at least 2.6% of the codon pairs in the ORF are codon pairs shown in Table 1.

Embodiment 27 is the polynucleotide of any one of embodiments 1-10, wherein at least 2.7% of the codon pairs in the ORF are codon pairs shown in Table 1.

Embodiment 28 is the polynucleotide of any one of embodiments 1-10, wherein at least 2.8% of the codon pairs in the ORF are codon pairs shown in Table 1.

Embodiment 29 is the polynucleotide of any one of embodiments 1-10, wherein at least 2.9% of the codon pairs in the ORF are codon pairs shown in Table 1.

Embodiment 30 is the polynucleotide of any one of embodiments 1-10, wherein at least 3.0% of the codon pairs in the ORF are codon pairs shown in Table 1.

Embodiment 31 is the polynucleotide of any one of embodiments 1-10, wherein at least 3.1% of the codon pairs in the ORF are codon pairs shown in Table 1.

Embodiment 32 is the polynucleotide of any one of embodiments 1-10, wherein at least 3.2% of the codon pairs in the ORF are codon pairs shown in Table 1.

Embodiment 33 is the polynucleotide of any one of embodiments 1-10, wherein at least 3.3% of the codon pairs in the ORF are codon pairs shown in Table 1.

Embodiment 34 is the polynucleotide of any one of embodiments 1-10, wherein at least 3.4% of the codon pairs in the ORF are codon pairs shown in Table 1.

Embodiment 35 is the polynucleotide of any one of embodiments 1-10, wherein at least 3.5% of the codon pairs in the ORF are codon pairs shown in Table 1.

Embodiment 36 is the polynucleotide of any one of embodiments 1-10, wherein at least 3.6% of the codon pairs in the ORF are codon pairs shown in Table 1.

Embodiment 37 is the polynucleotide of any one of embodiments 1-10, wherein at least 3.7% of the codon pairs in the ORF are codon pairs shown in Table 1.

Embodiment 38 is the polynucleotide of any one of embodiments 1-37, wherein less than or equal to 10% of the codon pairs in the ORF are codon pairs shown in Table 1.

Embodiment 39 is the polynucleotide of any one of embodiments 1-37, wherein less than or equal to 9.9% of the codon pairs in the ORF are codon pairs shown in Table 1.

Embodiment 40 is the polynucleotide of any one of embodiments 1-37, wherein less than or equal to 9.8% of the codon pairs in the ORF are codon pairs shown in Table 1.

Embodiment 41 is the polynucleotide of any one of embodiments 1-37, wherein less than or equal to 9.7% of the codon pairs in the ORF are codon pairs shown in Table 1.

Embodiment 42 is the polynucleotide of any one of embodiments 1-37, wherein less than or equal to 9.6% of the codon pairs in the ORF are codon pairs shown in Table 1.

Embodiment 43 is the polynucleotide of any one of embodiments 1-37, wherein less than or equal to 9.5% of the codon pairs in the ORF are codon pairs shown in Table 1.

Embodiment 44 is the polynucleotide of any one of embodiments 1-37, wherein less than or equal to 9.4% of the codon pairs in the ORF are codon pairs shown in Table 1.

Embodiment 45 is the polynucleotide of any one of embodiments 1-37, wherein less than or equal to 9.3% of the codon pairs in the ORF are codon pairs shown in Table 1.

Embodiment 46 is the polynucleotide of any one of embodiments 1-37, wherein less than or equal to 9.2% of the codon pairs in the ORF are codon pairs shown in Table 1.

Embodiment 47 is the polynucleotide of any one of embodiments 1-37, wherein less than or equal to 9.1% of the codon pairs in the ORF are codon pairs shown in Table 1.

Embodiment 48 is the polynucleotide of any one of embodiments 1-37, wherein less than or equal to 9.0% of the codon pairs in the ORF are codon pairs shown in Table 1.

Embodiment 49 is the polynucleotide of any one of embodiments 1-37, wherein less than or equal to 8.9% of the codon pairs in the ORF are codon pairs shown in Table 1.

Embodiment 50 is the polynucleotide of any one of embodiments 1-37, wherein less than or equal to 8.8% of the codon pairs in the ORF are codon pairs shown in Table 1.

Embodiment 51 is the polynucleotide of any one of embodiments 1-37, wherein less than or equal to 8.7% of the codon pairs in the ORF are codon pairs shown in Table 1.

Embodiment 52 is the polynucleotide of any one of embodiments 1-37, wherein less than or equal to 8.6% of the codon pairs in the ORF are codon pairs shown in Table 1.

Embodiment 53 is the polynucleotide of any one of embodiments 1-37, wherein less than or equal to 8.5% of the codon pairs in the ORF are codon pairs shown in Table 1.

Embodiment 54 is the polynucleotide of any one of embodiments 1-37, wherein less than or equal to 8.4% of the codon pairs in the ORF are codon pairs shown in Table 1.

Embodiment 55 is the polynucleotide of any one of embodiments 1-37, wherein less than or equal to 8.3% of the codon pairs in the ORF are codon pairs shown in Table 1.

Embodiment 56 is the polynucleotide of any one of embodiments 1-37, wherein less than or equal to 8.2% of the codon pairs in the ORF are codon pairs shown in Table 1.

Embodiment 57 is the polynucleotide of any one of embodiments 1-37, wherein less than or equal to 8.1% of the codon pairs in the ORF are codon pairs shown in Table 1.

Embodiment 58 is the polynucleotide of any one of embodiments 1-37, wherein less than or equal to 8.0% of the codon pairs in the ORF are codon pairs shown in Table 1.

Embodiment 59 is the polynucleotide of any one of embodiments 1-37, wherein less than or equal to 7.9% of the codon pairs in the ORF are codon pairs shown in Table 1.

Embodiment 60 is the polynucleotide of any one of embodiments 1-37, wherein less than or equal to 7.8% of the codon pairs in the ORF are codon pairs shown in Table 1.

Embodiment 61 is the polynucleotide of any one of embodiments 1-37, wherein less than or equal to 7.7% of the codon pairs in the ORF are codon pairs shown in Table 1.

Embodiment 62 is the polynucleotide of any one of embodiments 1-37, wherein less than or equal to 7.6% of the codon pairs in the ORF are codon pairs shown in Table 1.

Embodiment 63 is the polynucleotide of any one of embodiments 1-37, wherein less than or equal to 7.5% of the codon pairs in the ORF are codon pairs shown in Table 1.

Embodiment 64 is the polynucleotide of any one of embodiments 1-37, wherein less than or equal to 7.4% of the codon pairs in the ORF are codon pairs shown in Table 1.

Embodiment 65 is the polynucleotide of any one of embodiments 1-37, wherein less than or equal to 7.3% of the codon pairs in the ORF are codon pairs shown in Table 1.

Embodiment 66 is the polynucleotide of any one of embodiments 1-37, wherein less than or equal to 7.2% of the codon pairs in the ORF are codon pairs shown in Table 1.

Embodiment 67 is the polynucleotide of any one of embodiments 1-37, wherein less than or equal to 7.1% of the codon pairs in the ORF are codon pairs shown in Table 1.

Embodiment 68 is the polynucleotide of any one of embodiments 1-37, wherein less than or equal to 7.0% of the codon pairs in the ORF are codon pairs shown in Table 1.

Embodiment 69 is the polynucleotide of any one of embodiments 1-37, wherein less than or equal to 6.9% of the codon pairs in the ORF are codon pairs shown in Table 1.

Embodiment 70 is the polynucleotide of any one of embodiments 1-37, wherein less than or equal to 6.8% of the codon pairs in the ORF are codon pairs shown in Table 1.

Embodiment 71 is the polynucleotide of any one of embodiments 1-37, wherein less than or equal to 6.7% of the codon pairs in the ORF are codon pairs shown in Table 1.

Embodiment 72 is the polynucleotide of any one of embodiments 1-37, wherein less than or equal to 6.6% of the codon pairs in the ORF are codon pairs shown in Table 1.

Embodiment 73 is the polynucleotide of any one of embodiments 1-37, wherein less than or equal to 6.5% of the codon pairs in the ORF are codon pairs shown in Table 1.

Embodiment 74 is the polynucleotide of any one of embodiments 1-37, wherein less than or equal to 6.4% of the codon pairs in the ORF are codon pairs shown in Table 1.

Embodiment 75 is the polynucleotide of any one of embodiments 1-37, wherein less than or equal to 6.32% of the codon pairs in the ORF are codon pairs shown in Table 1.

Embodiment 76 is the polynucleotide of any one of embodiments 1-75, wherein less than or equal to 0.9% of the codon pairs in the ORF are codon pairs shown in Table 2.

Embodiment 77 is the polynucleotide of any one of embodiments 1-75, wherein less than or equal to 0.8% of the codon pairs in the ORF are codon pairs shown in Table 2.

Embodiment 78 is the polynucleotide of any one of embodiments 1-75, wherein less than or equal to 0.7% of the codon pairs in the ORF are codon pairs shown in Table 2.

Embodiment 79 is the polynucleotide of any one of embodiments 1-75, wherein less than or equal to 0.6% of the codon pairs in the ORF are codon pairs shown in Table 2.

Embodiment 80 is the polynucleotide of any one of embodiments 1-75, wherein less than or equal to 0.5% of the codon pairs in the ORF are codon pairs shown in Table 2.

Embodiment 81 is the polynucleotide of any one of embodiments 1-75, wherein less than or equal to 0.45% of the codon pairs in the ORF are codon pairs shown in Table 2.

Embodiment 82 is the polynucleotide of any one of embodiments 1-75, wherein less than or equal to 0.4% of the codon pairs in the ORF are codon pairs shown in Table 2.

Embodiment 83 is the polynucleotide of any one of embodiments 1-75, wherein less than or equal to 0.3% of the codon pairs in the ORF are codon pairs shown in Table 2.

Embodiment 84 is the polynucleotide of any one of embodiments 1-75, wherein less than or equal to 0.2% of the codon pairs in the ORF are codon pairs shown in Table 2.

Embodiment 85 is the polynucleotide of any one of embodiments 1-75, wherein less than or equal to 0.1% of the codon pairs in the ORF are codon pairs shown in Table 2.

Embodiment 86 is the polynucleotide of any one of embodiments 1-75, wherein the ORF does not comprise codon pairs shown in Table 2.

Embodiment 87 is the polynucleotide of any one of embodiments 1-86, wherein the GC content of the ORF is greater than or equal to 56%.

Embodiment 88 is the polynucleotide of any one of embodiments 1-86, wherein the GC content of the ORF is greater than or equal to 56.5%.

Embodiment 89 is the polynucleotide of any one of embodiments 1-86, wherein the GC content of the ORF is greater than or equal to 57%.

Embodiment 90 is the polynucleotide of any one of embodiments 1-86, wherein the GC content of the ORF is greater than or equal to 57.5%.

Embodiment 91 is the polynucleotide of any one of embodiments 1-86, wherein the GC content of the ORF is greater than or equal to 58%.

Embodiment 92 is the polynucleotide of any one of embodiments 1-86, wherein the GC content of the ORF is greater than or equal to 58.5%.

Embodiment 93 is the polynucleotide of any one of embodiments 1-86, wherein the GC content of the ORF is greater than or equal to 59%.

Embodiment 94 is the polynucleotide of any one of embodiments 1-93, wherein the GC content of the ORF is less than or equal to 63%.

Embodiment 95 is the polynucleotide of any one of embodiments 1-93, wherein the GC content of the ORF is less than or equal to 62.6%.

Embodiment 96 is the polynucleotide of any one of embodiments 1-93, wherein the GC content of the ORF is less than or equal to 62.1%.

Embodiment 97 is the polynucleotide of any one of embodiments 1-93, wherein the GC content of the ORF is less than or equal to 61.6%.

Embodiment 98 is the polynucleotide of any one of embodiments 1-93, wherein the GC content of the ORF is less than or equal to 61.1%.

Embodiment 99 is the polynucleotide of any one of embodiments 1-93, wherein the GC content of the ORF is less than or equal to 60.6%.

Embodiment 100 is the polynucleotide of any one of embodiments 1-93, wherein the GC content of the ORF is less than or equal to 60.1%.

Embodiment 101 is the polynucleotide of any one of embodiments 1-93, wherein the GC content of the ORF is less than or equal to 59.6%.

Embodiment 102 is the polynucleotide of any one of embodiments 1-101, wherein the repeat content of the ORF is less than or equal to 23.2%.

Embodiment 103 is the polynucleotide of any one of embodiments 1-101, wherein the repeat content of the ORF is less than or equal to 23.1%.

Embodiment 104 is the polynucleotide of any one of embodiments 1-101, wherein the repeat content of the ORF is less than or equal to 23.0%.

Embodiment 105 is the polynucleotide of any one of embodiments 1-101, wherein the repeat content of the ORF is less than or equal to 22.9%.

Embodiment 106 is the polynucleotide of any one of embodiments 1-101, wherein the repeat content of the ORF is less than or equal to 22.8%.

Embodiment 107 is the polynucleotide of any one of embodiments 1-101, wherein the repeat content of the ORF is less than or equal to 22.7%.

Embodiment 108 is the polynucleotide of any one of embodiments 1-101, wherein the repeat content of the ORF is less than or equal to 22.6%.

Embodiment 109 is the polynucleotide of any one of embodiments 1-101, wherein the repeat content of the ORF is less than or equal to 22.5%.

Embodiment 110 is the polynucleotide of any one of embodiments 1-101, wherein the repeat content of the ORF is less than or equal to 22.4%.

Embodiment 111 is the polynucleotide of any one of embodiments 1-110, wherein the repeat content of the ORF is greater than or equal to 20%.

Embodiment 112 is the polynucleotide of any one of embodiments 1-110, wherein the repeat content of the ORF is greater than or equal to 20.5%.

Embodiment 113 is the polynucleotide of any one of embodiments 1-110, wherein the repeat content of the ORF is greater than or equal to 21%.

Embodiment 114 is the polynucleotide of any one of embodiments 1-110, wherein the repeat content of the ORF is greater than or equal to 21.5%.

Embodiment 115 is the polynucleotide of any one of embodiments 1-110, wherein the repeat content of the ORF is greater than or equal to 21.7%.

Embodiment 116 is the polynucleotide of any one of embodiments 1-110, wherein the repeat content of the ORF is greater than or equal to 21.9%.

Embodiment 117 is the polynucleotide of any one of embodiments 1-110, wherein the repeat content of the ORF is greater than or equal to 22.1%.

Embodiment 118 is the polynucleotide of any one of embodiments 1-110, wherein the repeat content of the ORF is greater than or equal to 22.2%.

Embodiment 119 is the polynucleotide of any one of embodiments 1-118, wherein less than or equal to 15% of the codons in the ORF are codons shown in Table 4.

Embodiment 120 is the polynucleotide of any one of embodiments 1-118, wherein less than or equal to 14.5% of the codons in the ORF are codons shown in Table 4.

Embodiment 121 is the polynucleotide of any one of embodiments 1-118, wherein less than or equal to 14% of the codons in the ORF are codons shown in Table 4.

Embodiment 122 is the polynucleotide of any one of embodiments 1-118, wherein less than or equal to 13.5% of the codons in the ORF are codons shown in Table 4.

Embodiment 123 is the polynucleotide of any one of embodiments 1-118, wherein less than or equal to 13% of the codons in the ORF are codons shown in Table 4.

Embodiment 124 is the polynucleotide of any one of embodiments 1-118, wherein less than or equal to 12.5% of the codons in the ORF are codons shown in Table 4.

Embodiment 125 is the polynucleotide of any one of embodiments 1-118, wherein less than or equal to 12% of the codons in the ORF are codons shown in Table 4.

Embodiment 126 is the polynucleotide of any one of embodiments 1-118, wherein less than or equal to 11.5% of the codons in the ORF are codons shown in Table 4.

Embodiment 127 is the polynucleotide of any one of embodiments 1-118, wherein less than or equal to 11% of the codons in the ORF are codons shown in Table 4.

Embodiment 128 is the polynucleotide of any one of embodiments 1-118, wherein less than or equal to 10.5% of the codons in the ORF are codons shown in Table 4.

Embodiment 129 is the polynucleotide of any one of embodiments 1-118, wherein less than or equal to 10% of the codons in the ORF are codons shown in Table 4.

Embodiment 130 is the polynucleotide of any one of embodiments 1-118, wherein less than or equal to 9.5% of the codons in the ORF are codons shown in Table 4.

Embodiment 131 is the polynucleotide of any one of embodiments 1-118, wherein less than or equal to 9% of the codons in the ORF are codons shown in Table 4.

Embodiment 132 is the polynucleotide of any one of embodiments 1-118, wherein less than or equal to 8.5% of the codons in the ORF are codons shown in Table 4.

Embodiment 133 is the polynucleotide of any one of embodiments 1-118, wherein less than or equal to 8% of the codons in the ORF are codons shown in Table 4.

Embodiment 134 is the polynucleotide of any one of embodiments 1-118, wherein less than or equal to 7.5% of the codons in the ORF are codons shown in Table 4.

Embodiment 135 is the polynucleotide of any one of embodiments 1-118, wherein less than or equal to 7% of the codons in the ORF are codons shown in Table 4.

Embodiment 136 is the polynucleotide of any one of embodiments 1-135, wherein at least 76% of the codons in the ORF are codons shown in Table 3.

Embodiment 137 is the polynucleotide of any one of embodiments 1-135, wherein at least 77% of the codons in the ORF are codons shown in Table 3.

Embodiment 138 is the polynucleotide of any one of embodiments 1-135, wherein at least 78% of the codons in the ORF are codons shown in Table 3.

Embodiment 139 is the polynucleotide of any one of embodiments 1-135, wherein at least 79% of the codons in the ORF are codons shown in Table 3.

Embodiment 140 is the polynucleotide of any one of embodiments 1-135, wherein at least 80% of the codons in the ORF are codons shown in Table 3.

Embodiment 141 is the polynucleotide of any one of embodiments 1-140, wherein less than or equal to 87% of the codons in the ORF are codons shown in Table 3.

Embodiment 142 is the polynucleotide of any one of embodiments 1-140, wherein less than or equal to 86% of the codons in the ORF are codons shown in Table 3.

Embodiment 143 is the polynucleotide of any one of embodiments 1-140, wherein less than or equal to 85% of the codons in the ORF are codons shown in Table 3.

Embodiment 144 is the polynucleotide of any one of embodiments 1-140, wherein less than or equal to 84% of the codons in the ORF are codons shown in Table 3.

Embodiment 145 is the polynucleotide of any one of embodiments 1-140, wherein less than or equal to 83% of the codons in the ORF are codons shown in Table 3.

Embodiment 146 is the polynucleotide of any one of embodiments 1-140, wherein less than or equal to 82% of the codons in the ORF are codons shown in Table 3.

Embodiment 147 is the polynucleotide of any one of embodiments 1-140, wherein less than or equal to 81% of the codons in the ORF are codons shown in Table 3.

Embodiment 148 is the polynucleotide of any one of embodiments 1-140, wherein less than or equal to 80% of the codons in the ORF are codons shown in Table 3.

Embodiment 149 is the polynucleotide of any one of embodiments 1-140, wherein less than or equal to 79% of the codons in the ORF are codons shown in Table 3.

Embodiment 150 is the polynucleotide of any one of embodiments 1-149, wherein the ORF has a uridine content ranging from its minimum uridine content to 101%, 102%, 103%, 105%, 110%, 115%, 120%, 125%, 130%, 135%, 140%, 145%, or 150% of the minimum uridine content.

Embodiment 151 is the polynucleotide of any one of embodiments 1-150, wherein the ORF has an A+U content ranging from its minimum A+U content to 101%, 102%, 103%, 105%, 110%, 115%, 120%, 125%, 130%, 135%, 140%, 145%, or 150% of the minimum A+U content.

Embodiment 152 is the polynucleotide of any one of embodiments 1-151, wherein the ORF has a GC content in the range of 55%-65%, such as 55%-57%, 57%-59%, 59-61%, 61-63%, or 63-65%.

Embodiment 153 is the polynucleotide of any one of embodiments 1-152, wherein the ORF has a repeat content ranging from its minimum repeat content to 101%, 102%, 103%, 105%, 110%, 115%, 120%, 125%, 130%, 135%, 140%, 145%, or 150% of the minimum repeat content.

Embodiment 154 is the polynucleotide of any one of embodiments 1-153, wherein the ORF has a repeat content of 22%-27%, such as 22%-23%, 22.3%-23%, 23%-24%, 24%-25%, 25%-26%, or 26%-27%.

Embodiment 155 is the polynucleotide of any one of embodiments 1-154, wherein the polypeptide has a length of 30 amino acids, optionally wherein the polypeptide has a length of at least 50 amino acids.

Embodiment 156 is the polynucleotide of any one of embodiments 1-154, wherein the polypeptide has a length of at least 100 amino acids.

Embodiment 157 is the polynucleotide of any one of embodiments 1-154, wherein the polypeptide has a length of at least 200 amino acids.

Embodiment 158 is the polynucleotide of any one of embodiments 1-154, wherein the polypeptide has a length of at least 300 amino acids.

Embodiment 159 is the polynucleotide of any one of embodiments 1-154, wherein the polypeptide has a length of at least 400 amino acids.

Embodiment 160 is the polynucleotide of any one of embodiments 1-154, wherein the polypeptide has a length of at least 500 amino acids.

Embodiment 161 is the polynucleotide of any one of embodiments 1-154, wherein the polypeptide has a length of at least 600 amino acids.

Embodiment 162 is the polynucleotide of any one of embodiments 1-154, wherein the polypeptide has a length of at least 700 amino acids.

Embodiment 163 is the polynucleotide of any one of embodiments 1-154, wherein the polypeptide has a length of at least 800 amino acids.

Embodiment 164 is the polynucleotide of any one of embodiments 1-154, wherein the polypeptide has a length of at least 900 amino acids.

Embodiment 165 is the polynucleotide of any one of embodiments 1-154, wherein the polypeptide has a length of at least 1000 amino acids.

Embodiment 166 is the polynucleotide of any one of embodiments 1-165, wherein the length of the polypeptide is less than or equal to 5000 amino acids.

Embodiment 167 is the polynucleotide of any one of embodiments 1-165, wherein the length of the polypeptide is less than or equal to 4500 amino acids.

Embodiment 168 is the polynucleotide of any one of embodiments 1-165, wherein the length of the polypeptide is less than or equal to 4000 amino acids.

Embodiment 169 is the polynucleotide of any one of embodiments 1-165, wherein the length of the polypeptide is less than or equal to 3500 amino acids.

Embodiment 170 is the polynucleotide of any one of embodiments 1-165, wherein the length of the polypeptide is less than or equal to 3000 amino acids.

Embodiment 171 is the polynucleotide of any one of embodiments 1-165, wherein the length of the polypeptide is less than or equal to 2500 amino acids.

Embodiment 172 is the polynucleotide of any one of embodiments 1-165, wherein the length of the polypeptide is less than or equal to 2000 amino acids.

Embodiment 173 is the polynucleotide of any one of embodiments 1-165, wherein the length of the polypeptide is less than or equal to 1500 amino acids.

Embodiment 174 is the polynucleotide of any one of embodiments 1-173, wherein the polypeptide comprises a sequence with at least 90%, 95%, 96%, 97%, 98%, 99%, 99.5%, or 100% identity to any one of SEQ ID NOs: 6-10, 29, 46, 69-73, 90-93, 96-99, 102-105, 108-111, 114-117, 120-123, 126-129, or 134-143.

Embodiment 175a is the polynucleotide of any one of embodiments 1-174, wherein the polynucleotide comprises a sequence with at least 90%, 95%, 96%, 97%, 98%, 99%, 99.5%, or 100% identity to any one of SEQ ID NO: 16.

Embodiment 175b is the polynucleotide of any one of embodiments 1-174, wherein the polynucleotide comprises a sequence with at least 90%, 95%, 96%, 97%, 98%, 99%, 99.5%, or 100% identity to any one of SEQ ID NO: 17.

Embodiment 175c is the polynucleotide of any one of embodiments 1-174, wherein the polynucleotide comprises a sequence with at least 90%, 95%, 96%, 97%, 98%, 99%, 99.5%, or 100% identity to any one of SEQ ID NO: 18.

Embodiment 175d is the polynucleotide of any one of embodiments 1-174, wherein the polynucleotide comprises a sequence with at least 90%, 95%, 96%, 97%, 98%, 99%, 99.5%, or 100% identity to any one of SEQ ID NO: 19.

Embodiment 175e is the polynucleotide of any one of embodiments 1-174, wherein the polynucleotide comprises a sequence with at least 90%, 95%, 96%, 97%, 98%, 99%, 99.5%, or 100% identity to any one of SEQ ID NO: 20.

Embodiment 175f is the polynucleotide of any one of embodiments 1-174, wherein the polynucleotide comprises a sequence with at least 90%, 95%, 96%, 97%, 98%, 99%, 99.5%, or 100% identity to any one of SEQ ID NO: 78.

Embodiment 175g is the polynucleotide of any one of embodiments 1-174, wherein the polynucleotide comprises a sequence with at least 90%, 95%, 96%, 97%, 98%, 99%, 99.5%, or 100% identity to any one of SEQ ID NO: 79.

Embodiment 175h is the polynucleotide of any one of embodiments 1-174, wherein the polynucleotide comprises a sequence with at least 90%, 95%, 96%, 97%, 98%, 99%, 99.5%, or 100% identity to any one of SEQ ID NO: 80.

Embodiment 175i is the polynucleotide of any one of embodiments 1-174, wherein the polynucleotide comprises a sequence with at least 90%, 95%, 96%, 97%, 98%, 99%, 99.5%, or 100% identity to any one of SEQ ID NO: 194.

Embodiment 175j is the polynucleotide of any one of embodiments 1-174, wherein the polynucleotide comprises a sequence with at least 90%, 95%, 96%, 97%, 98%, 99%, 99.5%, or 100% identity to any one of SEQ ID NO: 195.

Embodiment 175l is the polynucleotide of any one of embodiments 1-174, wherein the polynucleotide comprises a sequence with at least 90%, 95%, 96%, 97%, 98%, 99%, 99.5%, or 100% identity to any one of SEQ ID NO: 196.

Embodiment 175m is the polynucleotide of any one of embodiments 1-174, wherein the polynucleotide comprises a sequence with at least 90%, 95%, 96%, 97%, 98%, 99%, 99.5%, or 100% identity to any one of SEQ ID NO: 197.

Embodiment 175n is the polynucleotide of any one of embodiments 1-174, wherein the polynucleotide comprises a sequence with at least 90%, 95%, 96%, 97%, 98%, 99%, 99.5%, or 100% identity to any one of SEQ ID NO: 200.

Embodiment 175o is the polynucleotide of any one of embodiments 1-174, wherein the polynucleotide comprises a sequence with at least 90%, 95%, 96%, 97%, 98%, 99%, 99.5%, or 100% identity to any one of SEQ ID NO: 201.

Embodiment 176 is the polynucleotide of any one of embodiments 1-175o, wherein the ORF encodes an RNA-guided DNA binding agent.

Embodiment 177 is the polynucleotide of embodiment 176, wherein the RNA-guided DNA-binding agent has double-stranded endonuclease activity.

Embodiment 178 is the polynucleotide of embodiment 177, wherein the RNA-guided DNA-binding agent comprises a Cas cleavase.

Embodiment 179 is the polynucleotide of embodiment 176, wherein the RNA-guided DNA-binding agent has nickase activity.

Embodiment 180 is the polynucleotide of embodiment 179, wherein the RNA-guided DNA-binding agent comprises a Cas nickase.

Embodiment 181 is the polynucleotide of embodiment 176, wherein the RNA-guided DNA-binding agent comprises a dCas DNA binding domain.

Embodiment 182 is the polynucleotide of any one of embodiments 178, 180, or 181, wherein the Cas cleavase, Cas nickase, or dCas DNA binding domain is a Cas9 cleavase, Cas9 nickase, or dCas9 DNA binding domain.

Embodiment 183 is the polynucleotide of any one of embodiments 1-182, wherein the ORF encodes an S. pyogenes Cas9.

Embodiment 184 is the polynucleotide of any one of embodiments 1-183, wherein the ORF encodes an endonuclease.

Embodiment 185 is the polynucleotide of any one of embodiments 1-175, wherein the ORF encodes a serine protease inhibitor or Serpin family member.

Embodiment 186 is the polynucleotide of embodiment 185, wherein the ORF encodes a Serpin Family A Member 1.

Embodiment 187 is the polynucleotide of any one of embodiments 1-175, wherein the ORF encodes a hydroxylase; carbamoyltransferase; glucosylceramidase; galactosidase; dehydrogenase; receptor; or neurotransmitter receptor.

Embodiment 188 is the polynucleotide of any one of embodiments 1-175, wherein the ORF encodes a phenylalanine hydroxylase; an ornithine carbamoyltransferase; a fumarylacetoacetate hydrolase; a glucosylceramidase beta; an alpha galactosidase; a transthyretin; a glyceraldehyde-3-phosphate dehydrogenase; a gamma-aminobutyric acid (GABA) receptor subunit (such as a GABA Type A Receptor Delta Subunit).

Embodiment 189 is the polynucleotide of any one of embodiments 1-188, wherein the polynucleotide further comprises a 5′ UTR with at least 90% identity to any one of SEQ ID NOs: 177-181 or 190-192.

Embodiment 190 is the polynucleotide of any one of embodiments 1-189, wherein the polynucleotide further comprises a 3′ UTR with at least 90% identity to any one of SEQ ID NOs: 182-186 or 202-204.

Embodiment 191 is the polynucleotide of embodiment 189 or 190, wherein the polynucleotide further comprises a 5′ UTR and a 3′ UTR from the same source.

Embodiment 192 is the polynucleotide of any one of embodiments 1-191, wherein the polynucleotide further comprises a 5′ cap selected from Cap0, Cap1, and Cap2.

Embodiment 193 is the polynucleotide of any one of embodiments 1-192, wherein the open reading frame has codons that increase translation of the polynucleotide in a mammal.

Embodiment 194 is the polynucleotide of any one of embodiments 1-193, wherein the encoded polypeptide comprises a nuclear localization signal (NLS).

Embodiment 195 is the polynucleotide of embodiment 194, wherein the NLS is linked to the C-terminus of the polypeptide.

Embodiment 196 is the polynucleotide of embodiment 194, wherein the NLS is linked to the N-terminus of the polypeptide.

Embodiment 197 is the polynucleotide of any one of embodiments 194-196, wherein the NLS comprises a sequence having at least 80%, 85%, 90%, or 95% identity to any one of SEQ ID NOs: 163-176.

Embodiment 198 is the polynucleotide of any one of embodiments 194-196, wherein the NLS comprises the sequence of any one of SEQ ID NOs: 163-176.

Embodiment 199 is the polynucleotide of any one of embodiments 1-198, wherein the polypeptide encodes an RNA-guided DNA-binding agent and the RNA-guided DNA-binding agent further comprises a heterologous functional domain.

Embodiment 200 is the polynucleotide of embodiment 199, wherein the heterologous functional domain is a FokI nuclease.

Embodiment 201 is the polynucleotide of embodiment 199, wherein the heterologous functional domain is a transcriptional regulatory domain.

Embodiment 202 is the polynucleotide of any of embodiments 1-201, wherein at least 10%, at least 20%, at least 30%, at least 40%, at least 50%, at least 60%, at least 70%, at least 80%, at least 90%, at least 95%, at least 98%, at least 99%, or 100% of the uridine is substituted with a modified uridine.

Embodiment 203 is the polynucleotide of embodiment 202, wherein the modified uridine is one or more of N1-methyl-pseudouridine, pseudouridine, 5-methoxyuridine, or 5-iodouridine.

Embodiment 204 is the polynucleotide of embodiment 202, wherein the modified uridine is one or both of N1-methyl-pseudouridine or 5-methoxyuridine.

Embodiment 205 is the polynucleotide of embodiment 202, wherein the modified uridine is N1-methyl-pseudouridine.

Embodiment 206 is the polynucleotide of embodiment 202, wherein the modified uridine is 5-methoxyuridine.

Embodiment 207 is the polynucleotide of any one of embodiments 202-206, wherein 15% to 45%, 45% to 55%, 55% to 65%, 65% to 75%, 75% to 85%, 85% to 95%, or 90% to 100% of the uridine is substituted with the modified uridine, optionally wherein the modified uridine is N1-methyl-pseudouridine.

Embodiment 208 is the polynucleotide of any one of embodiments 202-207, wherein at least 20% or at least 30% of the uridine is substituted with the modified uridine.

Embodiment 209 is the polynucleotide of embodiment 208, wherein at least 80% or at least 90% of the uridine is substituted with the modified uridine.

Embodiment 210 is the polynucleotide of embodiment 208, wherein 100% uridine is substituted with the modified uridine.

Embodiment 211 is the polynucleotide of any one of embodiments 1-210, wherein the polynucleotide is an mRNA.

Embodiment 212 is the polynucleotide of any one of embodiments 1-211, wherein the polynucleotide is an expression construct comprising a promoter operably linked to the ORF.

Embodiment 213 is a plasmid comprising the expression construct of embodiment 212.

Embodiment 214 is a host cell comprising the expression construct of embodiment 212 or the plasmid of embodiment 213.

Embodiment 215 is a method of preparing an mRNA comprising contacting the expression construct of embodiment 212 or the plasmid of embodiment 213 with an RNA polymerase under conditions permissive for transcription of the mRNA.

Embodiment 216 is the method of embodiment 215, wherein the contacting step is performed in vitro.

Embodiment 217 is a method of expressing a polypeptide, comprising contacting a cell with the polynucleotide of any one of embodiments 1-212.

Embodiment 218 is the method of embodiment 217, wherein the cell is in a mammalian subject, optionally wherein the subject is human.

Embodiment 219 is the method of embodiment 217, wherein the cell is a cultured cell and/or the contacting is performed in vitro.

Embodiment 220 is the method of any one of embodiments 217-219, wherein the cell is a human cell.

Embodiment 221 is a composition comprising a polynucleotide according to any one of embodiments 1-212 and at least one guide RNA, wherein the polynucleotide encodes an RNA-guided DNA binding agent.

Embodiment 222 is a lipid nanoparticle comprising a polynucleotide according to any one of embodiments 1-212.

Embodiment 223 is a pharmaceutical composition comprising a polynucleotide according to any one of embodiments 1-212 and a pharmaceutically acceptable carrier.

Embodiment 224 is the lipid nanoparticle of embodiment 222 or the pharmaceutical composition of embodiment 223, wherein the polynucleotide encodes an RNA-guided DNA binding agent and the lipid nanoparticle or pharmaceutical composition further comprises at least one guide RNA.

Embodiment 225 is a method of genome editing or modifying a target gene comprising contacting a cell with the polynucleotide, expression construct, composition, or lipid nanoparticle according to any one of embodiments 1-212 or 222-224, wherein the polynucleotide encodes an RNA-guided DNA binding agent.

Embodiment 226 is use of the polynucleotide, expression construct, composition, or lipid nanoparticle according to any one of embodiments 1-212 or 222-224 for genome editing or modifying a target gene, wherein the polynucleotide encodes an RNA-guided DNA binding agent.

Embodiment 227 is use of the polynucleotide, expression construct, composition, or lipid nanoparticle according to any one of embodiments 1-212 or 222-224 for the manufacture of a medicament for genome editing or modifying a target gene, wherein the polynucleotide encodes an RNA-guided DNA binding agent.

Embodiment 228 is the method or use of any one of embodiments 225-227, wherein the genome editing or modification of the target gene occurs in a liver cell.

Embodiment 229 is the method or use of embodiment 228, wherein the liver cell is a hepatocyte.

Embodiment 230 is the method or use of any one of embodiments 225-227, wherein the genome editing or modification of the target gene is in vivo.

Embodiment 231 is the method or use of any one of embodiments 225-227, wherein the genome editing or modification of the target gene is in an isolated or cultured cell.

Embodiment 232 is a method of generating an open reading frame (ORF) sequence encoding a polypeptide, the method comprising:
a) providing a polypeptide sequence of interest;
b) assigning a codon for each amino acid position of the polypeptide sequence, wherein if the amino acid position is a member of a dipeptide shown in Table 1, then the codon pair for that dipeptide is used, but if the amino acid position is a member of more than one dipeptide shown in Table 1 and the codon pairs for those dipeptides provide different codons for the position or the amino acid position is not a member of a dipeptide shown in Table 1, then one or more of the following is performed:
   i. selecting a codon from a wild-type sequence encoding the polypeptide if a naturally occurring polypeptide is encoded;
   ii. if the amino acid is a member of more than one dipeptide shown in Table 1 and the codon pairs for those dipeptides provide different codons for the position, eliminating codons that appear in Table 4 and/or that would result in the presence of a codon pair shown in Table 2, and/or selecting a codon that appears in Table 3;
   iii. using a codon set of Table 5, 6, or 7 to supply the codon for the amino acid position, optionally wherein if steps (i) and/or (ii) are performed then step (iii) is performed if a unique codon for the amino acid position has not been provided; and/or
   iv. selecting a codon that (1) minimizes uridine content, (2) minimizes repeat content, and/or (3) maximizes GC content.
   Embodiment 233 is the method of embodiment 232, wherein for at least one amino acid, Table 1 does not provide a unique codon at a given amino acid position, optionally wherein there are (1) conflicting codons in overlapping dipeptides; (2) multiple possible codons that corresponds to a given dipeptide; or (3) no codon that corresponds to a given dipeptide.

Embodiment 234 is the method of embodiment 232 or 233, wherein step (b)(ii) comprises performing one or more of the following:
a. selecting a codon that appears in Table 3; and/or
b. eliminating codon(s) that would result in the presence of a codon pair in
Table 2 and/or codon(s) that appear in Table 4, wherein one or more of the above steps are performed in any order and the steps are terminated when a single codon for the amino acid is provided.
Embodiment 235 is the method of any one of embodiments 232-234, wherein step (b)(ii) comprises selecting a codon that appears in Table 3, optionally wherein if one or more steps of embodiment 234 are performed, then the one or more steps of embodiment 234 are performed in any order relative to selecting a codon that appears in Table 3.

Embodiment 236 is the method of any one of embodiments 232-235, wherein step (b)(ii) further comprises:
a. eliminating codons that would result in the presence of a codon pair in Table 2; and
b. if more than one possible codon remains after step (a), eliminating codons that do not appear in Table 3 and/or eliminating codons that appear in Table 4.

Embodiment 237 is the method of any one of embodiments 232-236, wherein step (b)(ii) further comprises:
a. eliminating codons that do not appear in Table 3 and/or eliminating codons that appear in Table 4; and
b. if more than one possible codon remains after step (a), eliminating codons that would result in the presence of a codon pair in Table 2.

Embodiment 238 is the method of any one of embodiments 232-237, wherein step (b) comprises performing one or more of the following:
a. selecting the codon that minimizes uridine content;
b. selecting the codon that minimizes repeat content;
c. selecting the codon that maximizes GC content,
   wherein one or more of the above steps are performed in any order, optionally wherein the steps are terminated when a single codon for the amino acid is provided.

Embodiment 239 is the method of embodiment 238, wherein step (b) comprises performing at least one of the following and continuing to perform the following steps, optionally wherein each of the following steps (i)-(iii) is performed:
i. selecting the codon that minimizes uridine content;
ii. if more than one possible codon remains after step (a), selecting the codon that minimizes repeat content;
iii. if more than one possible codon remains after step (b), Selecting the codon that maximizes GC content.

Embodiment 240 is the method of any one of embodiments 232-239, wherein no codons remain after performing step (b)(ii) for at least one position that can be encoded by more than one codon, and the following steps are performed on a plurality of codons that encode the amino acid at the position:
i. selecting the codon that minimizes uridine content;
ii. if more than one possible codon remains after step (i), selecting the codon that minimizes repeat content;
iii. if more than one possible codon remains after step (ii), selecting the codon that maximizes GC content.

Embodiment 241 is the method of any one of embodiments 232-240, wherein a plurality of codons remain after performing step (b)(ii) for at least one position that can be encoded by more than one codon, and the following steps are performed on the plurality of codons:
i. selecting the codon that minimizes uridine content;
ii. if more than one possible codon remains after step (i), selecting the codon that minimizes repeat content;
iii. if more than one possible codon remains after step (ii), selecting the codon that maximizes GC content.

Embodiment 242 is the method of embodiments 240 or 241, wherein the method comprises selecting the codon that maximizes GC content in at least one position.

Embodiment 243 is the method of any one of embodiments 232-243, further comprising selecting a one-to-one codon set shown in Table 5, 6, or 7, and assigning a codon for at least one position from the set.

Embodiment 244 is the method of any one of embodiments 232-243, further comprising:
a. generating a set of all available codons for the amino acid to be encoded by at least one position;
b. applying one or more of the steps recited in embodiments 233-243.

Embodiment 245 is the method of any one of embodiments 232-244, wherein at least step (b) of the method is computer-implemented.

Embodiment 246 is the method of any one of embodiments 232-245, further comprising synthesizing a polynucleotide comprising the ORF, optionally wherein the polynucleotide is an mRNA.

Embodiment 247 is the method of any one of embodiments 232-246, wherein the RNA-guided DNA-binding agent has double-stranded endonuclease activity.

Embodiment 248 is the method of embodiment 247, wherein the RNA-guided DNA-binding agent comprises a Cas cleavase.

Embodiment 249 is the method of embodiment 247 or 248, wherein the RNA-guided DNA-binding agent has nickase activity.

Embodiment 250 is the method of embodiment 249, wherein the RNA-guided DNA-binding agent comprises a Cas nickase.

Embodiment 251 is the method of any one of embodiments 247-250, wherein the RNA-guided DNA-binding agent comprises a dCas DNA binding domain.

Embodiment 252 is the method of any one of embodiments 247-251, wherein the Cas cleavase, Cas nickase, or dCas DNA binding domain is a Cas9 cleavase, Cas9 nickase, or dCas9 DNA binding domain.

Embodiment 253 is the method of any one of embodiments 247-252, wherein the ORF encodes an S. pyogenes Cas9.

Embodiment 254 is the method of any one of embodiments 232-253, wherein the ORF encodes an endonuclease.

Embodiment 255 is the method of any one of embodiments 232-246, wherein the ORF encodes a serine protease inhibitor or Serpin family member.

Embodiment 256 is the method of embodiment 255, wherein the ORF encodes a Serpin Family A Member 1.

Embodiment 257 is the method of any one of embodiments 232-246, wherein the ORF encodes a hydroxylase; carbamoyltransferase; glucosylceramidase; galactosidase; dehydrogenase; receptor; or neurotransmitter receptor.

Embodiment 258 is the method of any one of embodiments 232-246, wherein the ORF encodes a phenylalanine hydroxylase; an ornithine carbamoyltransferase; a fumarylacetoacetate hydrolase; a glucosylceramidase beta; an alpha galactosidase; a transthyretin; a glyceraldehyde-3-phosphate dehydrogenase; a gamma-aminobutyric acid (GABA) receptor subunit (such as a GABA Type A Receptor Delta Subunit).

Embodiment 259 is the method of any one of embodiments 232-246, wherein the ORF encodes a polypeptide having at least 90% identity the amino acid sequence of any one of SEQ ID NOs: 1, 74, 88, 94, 100, 106, 112, 118, 124, 130, 161, or 162.

Embodiment 260 is the method of any one of embodiments 232-246, wherein the ORF encodes a polypeptide having at least 95% identity the amino acid sequence of any one of SEQ ID NOs: 1, 74, 88, 94, 100, 106, 112, 118, 124, 130, 161, or 162.

Embodiment 261 is the method of any one of embodiments 232-246, wherein the ORF encodes a polypeptide having at least 97% identity the amino acid sequence of any one of SEQ ID NOs: 1, 74, 88, 94, 100, 106, 112, 118, 124, 130, 161, or 162.

Embodiment 262 is the method of any one of embodiments 232-246, wherein the ORF encodes a polypeptide having at least 98% identity the amino acid sequence of any one of SEQ ID NOs: 1, 74, 88, 94, 100, 106, 112, 118, 124, 130, 161, or 162.

Embodiment 263 is the method of any one of embodiments 232-246, wherein the ORF encodes a polypeptide having at least 99% identity the amino acid sequence of any one of SEQ ID NOs: 1, 74, 88, 94, 100, 106, 112, 118, 124, 130, 161, or 162.

Embodiment 264 is the method of any one of embodiments 232-246, wherein the ORF encodes a polypeptide having at least 99.5% identity the amino acid sequence of any one of SEQ ID NOs: 1, 74, 88, 94, 100, 106, 112, 118, 124, 130, 161, or 162.

Embodiment 265 is the method of any one of embodiments 232-246, wherein the ORF encodes a polypeptide having 100% identity the amino acid sequence of any one of SEQ ID NOs: 1, 74, 88, 94, 100, 106, 112, 118, 124, 130, 161, or 162.

### BRIEF DESCRIPTION OF THE DRAWINGS

FIG. 1 shows expression of Cas9 in HepG2 cells 2, 6, and 24 hours after contacting the cells with mRNAs comprising the indicated sequences.

FIG. 2 shows expression of Cas9 in vivo using mRNAs comprising the indicated sequences.

FIG. 3 shows expression of Cas9 in vivo using mRNAs comprising the indicated sequences at 1, 3, and 6 hours after administration.

FIGS. 4A-4B show % Editing of the TTR gene and serum TTR levels in vivo following administration of mRNAs comprising the indicated sequences at the indicated doses.

FIGS. 5A-5B show a comparison of hA1AT expression using the indicated hSERPINA1 mRNA sequences at 6 hours and 24 hours post transfection in Primary Mouse Hepatocytes (PMH) (FIG. 5A) and in Primary Cyno Hepatocytes (PCH) (FIG. 5B).

FIG. 6 shows expression of Cas9 in primary human hepatocytes using mRNAs comprising the indicated sequences at 6 hours post transfection.

FIGS. 7A-7B show expression of Cas9 in primary human hepatocytes using mRNAs comprising the indicated sequences at 6 hours post transfection.

### DETAILED DESCRIPTION OF CERTAIN EMBODIMENTS

Reference will now be made in detail to certain embodiments of the invention, examples of which are illustrated in the accompanying drawings. While the invention will be described in conjunction with the illustrated embodiments, it will be understood that they are not intended to limit the invention to those embodiments. On the contrary, the invention is intended to cover all alternatives, modifications, and equivalents, which may be included within the invention as defined by the appended claims.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a conjugate” includes a plurality of conjugates and reference to “a cell” includes a plurality of cells and the like.

Numeric ranges are inclusive of the numbers defining the range. Measured and measurable values are understood to be approximate, taking into account significant digits and the error associated with the measurement. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques. The term “about” or “approximately” means an acceptable error for a particular value as determined by one of ordinary skill in the art, which depends in part on how the value is measured or determined, or a degree of variation that does not substantially affect the properties of the described subject matter, e.g., within 10%, 5%, 2%, or 1%. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the above specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

The section headings used herein are for organizational purposes only and are not to be construed as limiting the desired subject matter in any way. In the event that any literature incorporated by reference contradicts the express content of this specification, including but not limited to a definition, the express content of this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### Definitions

Unless stated otherwise, the following terms and phrases as used herein are intended to have the following meanings:

The term “or combinations thereof” as used herein refers to all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “kit” refers to a packaged set of related components, such as one or more polynucleotides or compositions and one or more related materials such as delivery devices (e.g., syringes), solvents, solutions, buffers, instructions, or desiccants.

“Or” is used in the inclusive sense, i.e., equivalent to “and/or,” unless the context requires otherwise.

“Polynucleotide” and “nucleic acid” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). For general discussion see The Biochemistry of the Nucleic Acids 5-36, Adams et al., ed., 11^{th} ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). RNA and DNA have different sugar moieties and can differ by the presence of uracil or analogs thereof in RNA and thymine or analogs thereof in DNA.

“Polypeptide” as used herein refers to a multimeric compound comprising amino acid residues that can adopt a three-dimensional conformation. Polypeptides include but are not limited to enzymes, enzyme precursor proteins, regulatory proteins, structural proteins, receptors, nucleic acid binding proteins, antibodies, etc. Polypeptides may, but do not necessarily, comprise post-translational modifications, non-natural amino acids, prosthetic groups, and the like.

“Modified uridine” is used herein to refer to a nucleoside other than thymidine with the same hydrogen bond acceptors as uridine and one or more structural differences from uridine. In some embodiments, a modified uridine is a substituted uridine, i.e., a uridine in which one or more non-proton substituents (e.g., alkoxy, such as methoxy) takes the place of a proton. In some embodiments, a modified uridine is pseudouridine. In some embodiments, a modified uridine is a substituted pseudouridine, i.e., a pseudouridine in which one or more non-proton substituents (e.g., alkyl, such as methyl) takes the place of a proton. In some embodiments, a modified uridine is any of a substituted uridine, pseudouridine, or a substituted pseudouridine.

“Uridine position” as used herein refers to a position in a polynucleotide occupied by a uridine or a modified uridine. Thus, for example, a polynucleotide in which “100% of the uridine positions are modified uridines” contains a modified uridine at every position that would be a uridine in a conventional RNA (where all bases are standard A, U, C, or G bases) of the same sequence. Unless otherwise indicated, a U in a polynucleotide sequence of a sequence table or sequence listing in or accompanying this disclosure can be a uridine or a modified uridine.

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server are generally appropriate.

“mRNA” is used herein to refer to a polynucleotide that is RNA or modified RNA and comprises an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of an mRNA phosphate-sugar backbone consist essentially of ribose residues, 2′-methoxy ribose residues, or a combination thereof. In general, mRNAs do not contain a substantial quantity of thymidine residues (e.g., 0 residues or fewer than 30, 20, 10, 5, 4, 3, or 2 thymidine residues; or less than 10%, 9%, 8%, 7%, 6%, 5%, 4%, 4%, 3%, 2%, 1%, 0.5%, 0.2%, or 0.1% thymidine content). An mRNA can contain modified uridines at some or all of its uridine positions.

As used herein, an “RNA-guided DNA binding agent” means a polypeptide or complex of polypeptides having RNA and DNA binding activity, or a DNA-binding subunit of such a complex, wherein the DNA binding activity is sequence-specific and depends on the sequence of the RNA. Exemplary RNA-guided DNA binding agents include Cas cleavases/nickases and inactivated forms thereof (“dCas DNA binding agents”). “Cas nuclease”, also called “Cas protein”, as used herein, encompasses Cas cleavases, Cas nickases, and dCas DNA binding agents. Cas cleavases/nickases and dCas DNA binding agents include a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. As used herein, a “Class 2 Cas nuclease” is a single-chain polypeptide with RNA-guided DNA binding activity, such as a Cas9 nuclease or a Cpf1 nuclease. Class 2 Cas nucleases include Class 2 Cas cleavases and Class 2 Cas nickases (e.g., H840A, D10A, or N863A variants), which further have RNA-guided DNA cleavase or nickase activity, and Class 2 dCas DNA binding agents, in which cleavase/nickase activity is inactivated. Class 2 Cas nucleases include, for example, Cas9, Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9(1.0) (e.g., K810A, K1003A, R1060A variants), and eSPCas9(1.1) (e.g., K848A, K1003A, R1060A variants) proteins and modifications thereof. Cpf1 protein, Zetsche et al., Cell, 163: 1-13 (2015), is homologous to Cas9, and contains a RuvC-like nuclease domain. Cpf1 sequences of Zetsche are incorporated by reference in their entirety. See, e.g., Zetsche, Tables S1 and S3. “Cas9” encompasses Spy Cas9, the variants of Cas9 listed herein, and equivalents thereof. See, e.g., Makarova et al., Nat Rev Microbiol, 13(11): 722-36 (2015); Shmakov et al., Molecular Cell, 60:385-397 (2015).

As used herein, the “minimum uridine content” of a given open reading frame (ORF) is the uridine content of an ORF that (a) uses a minimal uridine codon at every position and (b) encodes the same amino acid sequence as the given ORF. The minimal uridine codon(s) for a given amino acid is the codon(s) with the fewest uridines (usually 0 or 1 except for a codon for phenylalanine, where the minimal uridine codon has 2 uridines). Modified uridine residues are considered equivalent to uridines for the purpose of evaluating minimum uridine content.

As used herein, the “minimum uridine dinucleotide content” of a given open reading frame (ORF) is the lowest possible uridine dinucleotide (UU) content of an ORF that (a) uses a minimal uridine codon (as discussed above) at every position and (b) encodes the same amino acid sequence as the given ORF. The uridine dinucleotide (UU) content can be expressed in absolute terms as the enumeration of UU dinucleotides in an ORF or on a rate basis as the percentage of positions occupied by the uridines of uridine dinucleotides (for example, AUUAU would have a uridine dinucleotide content of 40% because 2 of 5 positions are occupied by the uridines of a uridine dinucleotide). Modified uridine residues are considered equivalent to uridines for the purpose of evaluating minimum uridine dinucleotide content.

As used herein, the “minimum adenine content” of a given open reading frame (ORF) is the adenine content of an ORF that (a) uses a minimal adenine codon at every position and (b) encodes the same amino acid sequence as the given ORF. The minimal adenine codon(s) for a given amino acid is the codon(s) with the fewest adenines (usually 0 or 1 except for a codon for lysine and asparagine, where the minimal adenine codon has 2 adenines). Modified adenine residues are considered equivalent to adenines for the purpose of evaluating minimum adenine content.

As used herein, the “minimum adenine dinucleotide content” of a given open reading frame (ORF) is the lowest possible adenine dinucleotide (AA) content of an ORF that (a) uses a minimal adenine codon (as discussed above) at every position and (b) encodes the same amino acid sequence as the given ORF. The adenine dinucleotide (AA) content can be expressed in absolute terms as the enumeration of AA dinucleotides in an ORF or on a rate basis as the percentage of positions occupied by the adenines of adenine dinucleotides (for example, UAAUA would have an adenine dinucleotide content of 40% because 2 of 5 positions are occupied by the adenines of an adenine dinucleotide). Modified adenine residues are considered equivalent to adenines for the purpose of evaluating minimum adenine dinucleotide content.

As used herein, the “minimum repeat content” of a given open reading frame (ORF) is the minimum possible sum of occurrences of AA, CC, GG, and TT (or TU, UT, or UU) dinucleotides in an ORF that encodes the same amino acid sequence as the given ORF. The repeat content can be expressed in absolute terms as the enumeration of AA, CC, GG, and TT (or TU, UT, or UU) dinucleotides in an ORF or on a rate basis as the enumeration of AA, CC, GG, and TT (or TU, UT, or UU) dinucleotides in an ORF divided by the length in nucleotides of the ORF (for example, UAAUA would have a repeat content of 20% because one repeat occurs in a sequence of 5 nucleotides). Modified adenine, guanine, cytosine, thymine, and uracil residues are considered equivalent to adenine, guanine, cytosine, thymine, and uracil residues for the purpose of evaluating minimum repeat content.

“Guide RNA”, “gRNA”, and “guide” are used herein interchangeably to refer to either a crRNA (also known as CRISPR RNA), or the combination of a crRNA and a trRNA (also known as tracrRNA). The crRNA and trRNA may be associated as a single RNA molecule (single guide RNA, sgRNA) or in two separate RNA molecules (dual guide RNA, dgRNA). “Guide RNA” or “gRNA” refers to each type. The trRNA may be a naturally-occurring sequence, or a trRNA sequence with modifications or variations compared to naturally-occurring sequences. Guide RNAs can include modified RNAs as described herein.

As used herein, a “guide sequence” refers to a sequence within a guide RNA that is complementary to a target sequence and functions to direct a guide RNA to a target sequence for binding or modification (e.g., cleavage) by an RNA-guided DNA binding agent. A “guide sequence” may also be referred to as a “targeting sequence,” or a “spacer sequence.” A guide sequence can be 20 base pairs in length, e.g., in the case of Streptococcus pyogenes (i.e., Spy Cas9) and related Cas9 homologs/orthologs. Shorter or longer sequences can also be used as guides, e.g., 15-, 16-, 17-, 18-, 19-, 21-, 22-, 23-, 24-, or 25-nucleotides in length. In some embodiments, the target sequence is in a gene or on a chromosome, for example, and is complementary to the guide sequence. In some embodiments, the degree of complementarity or identity between a guide sequence and its corresponding target sequence may be about 75%, 80%, 85%, 90%, 95%, 96%, 97%, 98%, 99%, or 100%. In some embodiments, the guide sequence and the target region may be 100% complementary or identical. In other embodiments, the guide sequence and the target region may contain at least one mismatch. For example, the guide sequence and the target sequence may contain 1, 2, 3, or 4 mismatches, where the total length of the target sequence is at least 17, 18, 19, 20 or more base pairs. In some embodiments, the guide sequence and the target region may contain 1-4 mismatches where the guide sequence comprises at least 17, 18, 19, 20 or more nucleotides. In some embodiments, the guide sequence and the target region may contain 1, 2, 3, or 4 mismatches where the guide sequence comprises 20 nucleotides.

Target sequences for Cas proteins include both the positive and negative strands of genomic DNA (i.e., the sequence given and the sequence's reverse compliment), as a nucleic acid substrate for a Cas protein is a double stranded nucleic acid. Accordingly, where a guide sequence is said to be “complementary to a target sequence”, it is to be understood that the guide sequence may direct a guide RNA to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

As used herein, “indels” refer to insertion/deletion mutations consisting of a number of nucleotides that are either inserted or deleted at the site of double-stranded breaks (DSBs) in the nucleic acid.

As used herein, “knockdown” refers to a decrease in expression of a particular gene product (e.g., protein, mRNA, or both). Knockdown of a protein can be measured either by detecting protein secreted by tissue or population of cells (e.g., in serum or cell media) or by detecting total cellular amount of the protein from a tissue or cell population of interest. Methods for measuring knockdown of mRNA are known and include sequencing of mRNA isolated from a tissue or cell population of interest. In some embodiments, “knockdown” may refer to some loss of expression of a particular gene product, for example a decrease in the amount of mRNA transcribed or a decrease in the amount of protein expressed or secreted by a population of cells (including in vivo populations such as those found in tissues).

As used herein, “knockout” refers to a loss of expression of a particular protein in a cell. Knockout can be measured either by detecting the amount of protein secretion from a tissue or population of cells (e.g., in serum or cell media) or by detecting total cellular amount of a protein a tissue or a population of cells. In some embodiments, the methods of the disclosure “knockout” a target protein one or more cells (e.g., in a population of cells including in vivo populations such as those found in tissues). In some embodiments, a knockout is not the formation of mutant of the target protein, for example, created by indels, but rather the complete loss of expression of the target protein in a cell.

As used herein, “ribonucleoprotein” (RNP) or “RNP complex” refers to a guide RNA together with an RNA-guided DNA binding agent, such as a Cas cleavase, nickase, or dCas DNA binding agent (e.g., Cas9). In some embodiments, the guide RNA guides the RNA-guided DNA binding agent such as Cas9 to a target sequence, and the guide RNA hybridizes with and the agent binds to the target sequence; in cases where the agent is a cleavase or nickase, binding can be followed by cleaving or nicking.

As used herein, a “target sequence” refers to a sequence of nucleic acid in a target gene that has complementarity to the guide sequence of the gRNA. The interaction of the target sequence and the guide sequence directs an RNA-guided DNA binding agent to bind, and potentially nick or cleave (depending on the activity of the agent), within the target sequence.

As used herein, “treatment” refers to any administration or application of a therapeutic for disease or disorder in a subject, and includes inhibiting the disease, arresting its development, relieving one or more symptoms of the disease, curing the disease, or preventing reoccurrence of one or more symptoms of the disease.

As used herein, the term “lipid nanoparticle” (LNP) refers to a particle that comprises a plurality of (i.e., more than one) lipid molecules physically associated with each other by intermolecular forces. The LNPs may be, e.g., microspheres (including unilamellar and multilamellar vesicles, e.g., “liposomes”—lamellar phase lipid bilayers that, in some embodiments, are substantially spherical—and, in more particular embodiments, can comprise an aqueous core, e.g., comprising a substantial portion of RNA molecules), a dispersed phase in an emulsion, micelles, or an internal phase in a suspension. Emulsions, micelles, and suspensions may be suitable compositions for local and/or topical delivery. See also, e.g., WO2017173054A1, the contents of which are hereby incorporated by reference in their entirety. Any LNP known to those of skill in the art to be capable of delivering nucleotides to subjects may be utilized with the guide RNAs and the nucleic acid encoding an RNA-guided DNA binding agent described herein.

As used herein, the terms “nuclear localization signal” (NLS) or “nuclear localization sequence” refers to an amino acid sequence which induces transport of molecules comprising such sequences or linked to such sequences into the nucleus of eukaryotic cells. The nuclear localization signal may form part of the molecule to be transported. In some embodiments, the NLS may be linked to the remaining parts of the molecule by covalent bonds, hydrogen bonds or ionic interactions.

As used herein, the phrase “pharmaceutically acceptable” means that which is useful in preparing a pharmaceutical composition that is generally non-toxic and is not biologically undesirable and that are not otherwise unacceptable for pharmaceutical use.

### A. Exemplary Polynucleotides and Compositions

### 1. ORF Codon Pair, Codon, and Repeat Content

Certain ORFs are translated in vivo more efficiently than others in terms of polypeptide molecules produced per mRNA molecule. It was hypothesized that the codon pair usage of such efficiently translated ORFs may contribute to translation efficiency.

Accordingly, a set of efficiently translated ORFs was identified by comparing mRNA and protein abundance data from human cells and selecting genes with high protein-to-mRNA abundance ratios. As a foil, a set of inefficiently translated ORFs was identified in a similar way, except that genes with low protein-to-mRNA ratios were selected. These sets were analyzed to determine significantly enriched codon pairs in the efficiently and inefficiently translated ORFs.

Tables 1 and 2 show the codon pairs so identified as enriched in the efficiently and inefficiently translated ORFs, respectively. The same sets were further analyzed to determine significantly enriched individual codons in the efficiently and inefficiently translated ORFs. Tables 3 and 4 show the codons so identified as enriched in the efficiently and inefficiently translated ORFs, respectively.

TABLE 1
Codon pairs enriched in efficiently
translated ORFs

| First | Second | | First | Second | |
|---|---|---|---|---|---|
| amino | amino | Codon | amino | amino | Codon |
| acid | acid | pair | acid | acid | pair |
| A | A | GCUGCC | L | P | CUUCCA |
| P | D | CCUGAC | P | P | CCACCC |
| Q | D | CAAGAC | T | P | ACUCCC |
| A | E | GCGGAG | T | Q | ACUCAA |
| P | E | CCAGAG | A | R | GCCCGC |
| R | E | AGGGAG | L | R | CUGCGG |
| W | E | UGGGAG | G | S | GGUAGC |
| C | G | UGUGGG | L | S | CUCAGC |
| G | G | GGUGGC | P | T | CCCACC |
| K | G | AAGGGG | S | T | UCGACU |
| P | G | CCUGGC | A | V | GCUGUG |
| Q | G | CAGGGC | E | V | GAAGUC |
| V | G | GUUGGC | Q | V | CAGGUG |
| M | I | AUGAUA | T | Y | ACCUAC |

TABLE 2
Codon pairs enriched in inefficiently
translated ORFs

| First | Second | | First | Second | |
|---|---|---|---|---|---|
| amino | amino | Codon | amino | amino | Codon |
| acid | acid | pair | acid | acid | pair |
| A | A | GCUGCU | W | H | UGGCAU |
| G | A | GGUGCU | E | L | GAGCUA |
| K | A | AAGGCU | Q | L | CAGUUG |
| P | A | CCAGCU | R | L | CGUCUU |
| Q | A | CAGGCU | V | L | GUGCUA |
| P | D | CCUGAU | A | P | GCACCA |
| Q | D | CAAGAU | G | P | GGACCA |
| R | D | CGAGAU | I | P | AUCCCU |
| A | E | GCGGAA | L | P | CUUCCU |
| A | E | GCAGAA | T | P | ACUCCA |
| G | E | GGUGAA | W | P | UGGCCU |
| P | E | CCAGAA | T | Q | ACUCAG |
| Q | E | CAGGAA | E | R | GAGAGA |
| R | E | AGGGAA | L | R | CUGAGA |
| T | E | ACAGAA | P | R | CCAAGA |
| W | E | UGGGAA | P | R | CCCAGA |
| A | G | GCUGGA | S | S | AGCUCU |
| G | G | GGUGGU | R | T | CGCACU |
| K | G | AAGGGU | E | V | GAGGUU |
| P | G | CCUGGU | P | V | CCUGUU |
| P | G | CCAGGA | Q | V | CAGGUU |
| P | G | CCUGGA | V | V | GUGGUU |
| V | G | GUAGGA | T | Y | ACCUAU |

TABLE 3
Codons enriched in efficiently translated ORFs

| Amino acid | Codon |
|---|---|
| A | GCC |
| A | GCG |
| C | UGC |
| D | GAC |
| E | GAG |
| F | UUC |
| G | GGC |
| G | GGG |
| H | CAC |
| I | AUA |
| I | AUC |
| L | CUC |
| L | CUG |
| N | AAC |
| P | CCC |
| P | CCG |
| Q | CAG |
| R | CGC |
| R | CGG |
| S | AGC |
| T | ACC |
| T | ACG |
| V | GUC |
| V | GUG |
| Y | UAC |

TABLE 4
Codons enriched in inefficiently translated ORFs

| Amino acid | Codon |
|---|---|
| [stop codon] | UAA |
| A | GCA |
| A | GCU |
| C | UGU |
| D | GAU |
| E | GAA |
| F | UUU |
| G | GGA |
| G | GGU |
| H | CAU |
| I | AUU |
| L | CUA |
| L | CUU |
| L | UUA |
| L | UUG |
| N | AAU |
| P | CCA |
| P | CCU |
| Q | CAA |
| R | AGA |
| R | CGA |
| R | CGU |
| S | AGU |
| S | UCU |
| T | ACA |
| T | ACU |
| V | GUA |
| V | GUU |
| Y | UAU |

In some embodiments, a polynucleotide is provided that comprises an open reading frame (ORF) encoding a polypeptide having a length of at least 30 amino acids, wherein at least 1%, at least 2%, at least 3%, or at least 4% of the codon pairs in the ORF are codon pairs shown in Table 1. In some embodiments, the polypeptide length and codon pair content are as set forth elsewhere herein, e.g., in the introduction and summary section above.

In some embodiments, a polynucleotide is provided that comprises an open reading frame (ORF) encoding a polypeptide having a length of at least 30 amino acids, wherein at least 1.03% of the codon pairs in the ORF are codon pairs shown in Table 1. In some embodiments, the polypeptide length and codon pair content are as set forth elsewhere herein, e.g., in the introduction and summary section above.

In some embodiments, a polynucleotide is provided that comprises an open reading frame (ORF) encoding a polypeptide having a length of at least 30 amino acids, wherein less than or equal to 1% of the codon pairs in the ORF are codon pairs shown in Table 2, optionally further wherein at least 1%, at least 2%, at least 3%, or at least 4% of the codon pairs in the ORF are codon pairs shown in Table 1. In some embodiments, the polypeptide length and codon pair content are as set forth elsewhere herein, e.g., in the introduction and summary section above.

In some embodiments, a polynucleotide is provided that comprises an open reading frame (ORF) encoding a polypeptide having a length of at least 30 amino acids, wherein less than or equal to 0.9% of the codon pairs in the ORF are codon pairs shown in Table 2, optionally further wherein at least 1.03% of the codon pairs in the ORF are codon pairs shown in Table 1. In some embodiments, the polypeptide length and codon pair content are as set forth elsewhere herein, e.g., in the introduction and summary section above.

In some embodiments, a polynucleotide is provided that comprises an open reading frame (ORF) encoding a polypeptide having a length of at least 30 amino acids, wherein at least 75%, at least 76%, at least 77%, at least 78%, at least 79%, at least 80% of the codons in the ORF are codons shown in Table 3, optionally further wherein at least 1%, at least 2%, at least 3%, or at least 4% of the codon pairs in the ORF are codon pairs shown in Table 1. In some embodiments, the polypeptide length and codon and codon pair content are as set forth elsewhere herein, e.g., in the introduction and summary section above.

In some embodiments, a polynucleotide is provided that comprises an open reading frame (ORF) encoding a polypeptide having a length of at least 30 amino acids, wherein at least 60%, 65%, 70%, 75% or 76% of the codons in the ORF are codons shown in Table 3, optionally further wherein at least 1.03% of the codon pairs in the ORF are codon pairs shown in Table 1, or wherein at least 1% of the codon pairs in the ORF are codon pairs shown in Table 1 and the ORF does not encode an RNA-guided DNA binding agent. In some embodiments, the polypeptide length and codon and codon pair content are as set forth elsewhere herein, e.g., in the introduction and summary section above.

In some embodiments, a polynucleotide is provided that comprises an open reading frame (ORF) encoding a polypeptide having a length of at least 30 amino acids, wherein less than or equal to 20%, less than or equal to 15%, less than or equal to 10%, less than or equal to 5% of the codons in the ORF are codons shown in Table 4, optionally further wherein optionally further wherein at least 1%, at least 2%, at least 3%, or at least 4% of the codon pairs in the ORF are codon pairs shown in Table 1. In some embodiments, the polypeptide length and codon and codon pair content are as set forth elsewhere herein, e.g., in the introduction and summary section above.

In some embodiments, a polynucleotide is provided that comprises an open reading frame (ORF) encoding a polypeptide having a length of at least 30 amino acids, wherein less than or equal to 15% of the codons in the ORF are codons shown in Table 4, optionally further wherein at least 1.03% of the codon pairs in the ORF are codon pairs shown in Table 1. In some embodiments, the polypeptide length and codon and codon pair content are as set forth elsewhere herein, e.g., in the introduction and summary section above.

In some embodiments, at least 1.05% of the codon pairs in the ORF are codon pairs shown in Table 1. In some embodiments, at least 1.1% of the codon pairs in the ORF are codon pairs shown in Table 1. In some embodiments, at least 1.2% of the codon pairs in the ORF are codon pairs shown in Table 1. In some embodiments, at least 1.3% of the codon pairs in the ORF are codon pairs shown in Table 1. In some embodiments, at least 1.4% of the codon pairs in the ORF are codon pairs shown in Table 1. In some embodiments, at least 1.5% of the codon pairs in the ORF are codon pairs shown in Table 1. In some embodiments, at least 1.6% of the codon pairs in the ORF are codon pairs shown in Table 1. In some embodiments, at least 1.7% of the codon pairs in the ORF are codon pairs shown in Table 1. In some embodiments, at least 1.8% of the codon pairs in the ORF are codon pairs shown in Table 1. In some embodiments, at least 1.9% of the codon pairs in the ORF are codon pairs shown in Table 1. In some embodiments, at least 2.0% of the codon pairs in the ORF are codon pairs shown in Table 1. In some embodiments, at least 2.1% of the codon pairs in the ORF are codon pairs shown in Table 1. In some embodiments, at least 2.3% of the codon pairs in the ORF are codon pairs shown in Table 1. In some embodiments, at least 2.4% of the codon pairs in the ORF are codon pairs shown in Table 1. In some embodiments, at least 2.5% of the codon pairs in the ORF are codon pairs shown in Table 1. In some embodiments, at least 2.6% of the codon pairs in the ORF are codon pairs shown in Table 1. In some embodiments, at least 2.7% of the codon pairs in the ORF are codon pairs shown in Table 1. In some embodiments, at least 2.8% of the codon pairs in the ORF are codon pairs shown in Table 1. In some embodiments, at least 2.9% of the codon pairs in the ORF are codon pairs shown in Table 1. In some embodiments, at least 3.0% of the codon pairs in the ORF are codon pairs shown in Table 1. In some embodiments, at least 3.1% of the codon pairs in the ORF are codon pairs shown in Table 1. In some embodiments, at least 3.2% of the codon pairs in the ORF are codon pairs shown in Table 1. In some embodiments, at least 3.3% of the codon pairs in the ORF are codon pairs shown in Table 1. In some embodiments, at least 3.4% of the codon pairs in the ORF are codon pairs shown in Table 1. In some embodiments, at least 3.5% of the codon pairs in the ORF are codon pairs shown in Table 1. In some embodiments, at least 3.6% of the codon pairs in the ORF are codon pairs shown in Table 1. In some embodiments, at least 3.7% of the codon pairs in the ORF are codon pairs shown in Table 1.

In some embodiments, less than or equal to 10% of the codon pairs in the ORF are codon pairs shown in Table 1. In some embodiments, less than or equal to 9.9% of the codon pairs in the ORF are codon pairs shown in Table 1. In some embodiments, less than or equal to 9.8% of the codon pairs in the ORF are codon pairs shown in Table 1. In some embodiments, less than or equal to 9.7% of the codon pairs in the ORF are codon pairs shown in Table 1. In some embodiments, less than or equal to 9.6% of the codon pairs in the ORF are codon pairs shown in Table 1. In some embodiments, less than or equal to 9.5% of the codon pairs in the ORF are codon pairs shown in Table 1. In some embodiments, less than or equal to 9.4% of the codon pairs in the ORF are codon pairs shown in Table 1. In some embodiments, less than or equal to 9.3% of the codon pairs in the ORF are codon pairs shown in Table 1. In some embodiments, less than or equal to 9.2% of the codon pairs in the ORF are codon pairs shown in Table 1. In some embodiments, less than or equal to 9.1% of the codon pairs in the ORF are codon pairs shown in Table 1. In some embodiments, less than or equal to 9.0% of the codon pairs in the ORF are codon pairs shown in Table 1. In some embodiments, less than or equal to 8.9% of the codon pairs in the ORF are codon pairs shown in Table 1. In some embodiments, less than or equal to 8.8% of the codon pairs in the ORF are codon pairs shown in Table 1. In some embodiments, less than or equal to 8.7% of the codon pairs in the ORF are codon pairs shown in Table 1. In some embodiments, less than or equal to 8.6% of the codon pairs in the ORF are codon pairs shown in Table 1. In some embodiments, less than or equal to 8.5% of the codon pairs in the ORF are codon pairs shown in Table 1. In some embodiments, less than or equal to 8.4% of the codon pairs in the ORF are codon pairs shown in Table 1. In some embodiments, less than or equal to 8.3% of the codon pairs in the ORF are codon pairs shown in Table 1. In some embodiments, less than or equal to 8.2% of the codon pairs in the ORF are codon pairs shown in Table 1. In some embodiments, less than or equal to 8.1% of the codon pairs in the ORF are codon pairs shown in Table 1. In some embodiments, less than or equal to 8.0% of the codon pairs in the ORF are codon pairs shown in Table 1. In some embodiments, less than or equal to 7.9% of the codon pairs in the ORF are codon pairs shown in Table 1. In some embodiments, less than or equal to 7.8% of the codon pairs in the ORF are codon pairs shown in Table 1. In some embodiments, less than or equal to 7.7% of the codon pairs in the ORF are codon pairs shown in Table 1. In some embodiments, less than or equal to 7.6% of the codon pairs in the ORF are codon pairs shown in Table 1. In some embodiments, less than or equal to 7.5% of the codon pairs in the ORF are codon pairs shown in Table 1. In some embodiments, less than or equal to 7.4% of the codon pairs in the ORF are codon pairs shown in Table 1. In some embodiments, less than or equal to 7.3% of the codon pairs in the ORF are codon pairs shown in Table 1. In some embodiments, less than or equal to 7.2% of the codon pairs in the ORF are codon pairs shown in Table 1. In some embodiments, less than or equal to 7.1% of the codon pairs in the ORF are codon pairs shown in Table 1. In some embodiments, less than or equal to 7.0% of the codon pairs in the ORF are codon pairs shown in Table 1. In some embodiments, less than or equal to 6.9% of the codon pairs in the ORF are codon pairs shown in Table 1. In some embodiments, less than or equal to 6.8% of the codon pairs in the ORF are codon pairs shown in Table 1. In some embodiments, less than or equal to 6.7% of the codon pairs in the ORF are codon pairs shown in Table 1. In some embodiments, less than or equal to 6.6% of the codon pairs in the ORF are codon pairs shown in Table 1. In some embodiments, less than or equal to 6.5% of the codon pairs in the ORF are codon pairs shown in Table 1. In some embodiments, less than or equal to 6.4% of the codon pairs in the ORF are codon pairs shown in Table 1. In some embodiments, less than or equal to 6.32% of the codon pairs in the ORF are codon pairs shown in Table 1.

In some embodiments, less than or equal to 0.8% of the codon pairs in the ORF are codon pairs shown in Table 2. In some embodiments, less than or equal to 0.7% of the codon pairs in the ORF are codon pairs shown in Table 2. In some embodiments, less than or equal to 0.6% of the codon pairs in the ORF are codon pairs shown in Table 2. In some embodiments, less than or equal to 0.5% of the codon pairs in the ORF are codon pairs shown in Table 2. In some embodiments, less than or equal to 0.45% of the codon pairs in the ORF are codon pairs shown in Table 2. In some embodiments, less than or equal to 0.4% of the codon pairs in the ORF are codon pairs shown in Table 2. In some embodiments, less than or equal to 0.3% of the codon pairs in the ORF are codon pairs shown in Table 2. In some embodiments, less than or equal to 0.2% of the codon pairs in the ORF are codon pairs shown in Table 2. In some embodiments, less than or equal to 0.1% of the codon pairs in the ORF are codon pairs shown in Table 2. In some embodiments, the ORF does not comprise codon pairs shown in Table 2.

In some embodiments, less than or equal to 15% of the codons in the ORF are codons shown in Table 4. In some embodiments, less than or equal to 14.5% of the codons in the ORF are codons shown in Table 4. In some embodiments, less than or equal to 14% of the codons in the ORF are codons shown in Table 4. In some embodiments, less than or equal to 13.5% of the codons in the ORF are codons shown in Table 4. In some embodiments, less than or equal to 13% of the codons in the ORF are codons shown in Table 4. In some embodiments, less than or equal to 12.5% of the codons in the ORF are codons shown in Table 4. In some embodiments, less than or equal to 12% of the codons in the ORF are codons shown in Table 4. In some embodiments, less than or equal to 11.5% of the codons in the ORF are codons shown in Table 4. In some embodiments, less than or equal to 11% of the codons in the ORF are codons shown in Table 4. In some embodiments, less than or equal to 10.5% of the codons in the ORF are codons shown in Table 4. In some embodiments, less than or equal to 10% of the codons in the ORF are codons shown in Table 4. In some embodiments, less than or equal to 9.5% of the codons in the ORF are codons shown in Table 4. In some embodiments, less than or equal to 9% of the codons in the ORF are codons shown in Table 4. In some embodiments, less than or equal to 8.5% of the codons in the ORF are codons shown in Table 4. In some embodiments, less than or equal to 8% of the codons in the ORF are codons shown in Table 4. In some embodiments, less than or equal to 7.5% of the codons in the ORF are codons shown in Table 4. In some embodiments, less than or equal to 7% of the codons in the ORF are codons shown in Table 4.

In some embodiments, at least 77% of the codons in the ORF are codons shown in Table 3. In some embodiments, at least 78% of the codons in the ORF are codons shown in Table 3. In some embodiments, at least 79% of the codons in the ORF are codons shown in Table 3. In some embodiments, at least 80% of the codons in the ORF are codons shown in Table 3. In some embodiments, less than or equal to 87% of the codons in the ORF are codons shown in Table 3. In some embodiments, less than or equal to 86% of the codons in the ORF are codons shown in Table 3. In some embodiments, less than or equal to 85% of the codons in the ORF are codons shown in Table 3. In some embodiments, less than or equal to 84% of the codons in the ORF are codons shown in Table 3. In some embodiments, less than or equal to 83% of the codons in the ORF are codons shown in Table 3. In some embodiments, less than or equal to 82% of the codons in the ORF are codons shown in Table 3. In some embodiments, less than or equal to 81% of the codons in the ORF are codons shown in Table 3. In some embodiments, less than or equal to 80% of the codons in the ORF are codons shown in Table 3. In some embodiments, less than or equal to 79% of the codons in the ORF are codons shown in Table 3.

In some embodiments, a polynucleotide is provided that comprises an open reading frame (ORF) encoding a polypeptide having a length of at least 30 amino acids, wherein the repeat content of the ORF is 22%-27%, 22%-23%, 22.3%-23%, 23%-24%, 24%-25%, 25%-26%, or 26%-27%; greater than or equal to 20%, 21%, or 22%; less than or equal to 20%, 21%, or 22%, optionally further wherein at least 1%, at least 2%, at least 3%, or at least 4% of the codon pairs in the ORF are codon pairs shown in Table 1. In some embodiments, the polypeptide length, repeat, and codon pair content are as set forth elsewhere herein, e.g., in the introduction and summary section above.

In some embodiments, a polynucleotide is provided that comprises an open reading frame (ORF) encoding a polypeptide having a length of at least 30 amino acids, wherein the repeat content of the ORF is less than or equal to 23.3%, optionally further wherein at least 1.03% of the codon pairs in the ORF are codon pairs shown in Table 1. In some embodiments, the polypeptide length, repeat, and codon pair content are as set forth elsewhere herein, e.g., in the introduction and summary section above.

In some embodiments, a polynucleotide is provided that comprises an open reading frame (ORF) encoding a polypeptide having a length of at least 30 amino acids, wherein the GC content of the ORF is greater than or equal to 54%, 55%, 56%, 56%, 57%, 58%, 59%, 60%, or 61%; less than or equal to 64%, 63%, 62%, 61%, 60%, or 59%, optionally further wherein at least 1%, at least 2%, at least 3%, or at least 4% of the codon pairs in the ORF are codon pairs shown in Table 1. In some embodiments, the polypeptide length, repeat, and codon pair content are as set forth elsewhere herein, e.g., in the introduction and summary section above.

In some embodiments, a polynucleotide is provided that comprises an open reading frame (ORF) encoding a polypeptide having a length of at least 30 amino acids, wherein the GC content of the ORF is greater than or equal to 55%, optionally further wherein at least 1.03% of the codon pairs in the ORF are codon pairs shown in Table 1. In some embodiments, the polypeptide length, repeat, and codon pair content are as set forth elsewhere herein, e.g., in the introduction and summary section above.

In some embodiments, the repeat content of the ORF is greater than or equal to 20%. In some embodiments, the repeat content of the ORF is greater than or equal to 20.5%. In some embodiments, the repeat content of the ORF is greater than or equal to 21%. In some embodiments, the repeat content of the ORF is greater than or equal to 21.5%. In some embodiments, the repeat content of the ORF is greater than or equal to 21.7%. In some embodiments, the repeat content of the ORF is greater than or equal to 21.9%. In some embodiments, the repeat content of the ORF is greater than or equal to 22.1%. In some embodiments, the repeat content of the ORF is greater than or equal to 22.2%.

In some embodiments, the GC content of the ORF is greater than or equal to 56%. In some embodiments, the GC content of the ORF is greater than or equal to 56.5%. In some embodiments, the GC content of the ORF is greater than or equal to 57%. In some embodiments, the GC content of the ORF is greater than or equal to 57.5%. In some embodiments, the GC content of the ORF is greater than or equal to 58%. In some embodiments, the GC content of the ORF is greater than or equal to 58.5%. In some embodiments, the GC content of the ORF is greater than or equal to 59%. In some embodiments, the GC content of the ORF is less than or equal to 63%. In some embodiments, the GC content of the ORF is less than or equal to 62.6%. In some embodiments, the GC content of the ORF is less than or equal to 62.1%. In some embodiments, the GC content of the ORF is less than or equal to 61.6%. In some embodiments, the GC content of the ORF is less than or equal to 61.1%. In some embodiments, the GC content of the ORF is less than or equal to 60.6%. In some embodiments, the GC content of the ORF is less than or equal to 60.1%.

In some embodiments, the repeat content of the ORF is less than or equal to 59.6%. In some embodiments, the repeat content of the ORF is less than or equal to 23.2%. In some embodiments, the repeat content of the ORF is less than or equal to 23.1%. In some embodiments, the repeat content of the ORF is less than or equal to 23.0%. In some embodiments, the repeat content of the ORF is less than or equal to 22.9%. In some embodiments, the repeat content of the ORF is less than or equal to 22.8%. In some embodiments, the repeat content of the ORF is less than or equal to 22.7%. In some embodiments, the repeat content of the ORF is less than or equal to 22.6%. In some embodiments, the repeat content of the ORF is less than or equal to 22.5%. In some embodiments, the repeat content of the ORF is less than or equal to 22.4%.

It will be appreciated that, overall, there are 400 possible pairings of first and second amino acids, and significantly enriched codon pairs were not identified for all pairings. Furthermore, in some cases, there may be a conflict between overlapping dipeptide segments as to which amino acid should be used at a position which is the C-terminal position of a first dipeptide segment and the N-terminal segment of a second dipeptide segment, or there may be more than one possible enriched codon pair that corresponds to a given dipeptide segment. Therefore, to design a complete ORF, it will often be useful to employ one or more additional approaches to encode amino acids in pairs for which there is no enriched pair or which are subject to conflicts between overlapping dipeptides. A number of approaches are provided to determine appropriate codons in such situations. For example, one such approach is to use codons from a wild-type sequence, where a naturally occurring polypeptide is encoded. Another approach is to use one or more algorithmic steps to narrow down the possible codons for each amino acid. A third approach is to use a codon set that provides a specific codon for each amino acid.

Regarding algorithmic steps to narrow down the possible codons for each amino acid, one or more of the following steps may be applied for one or more (e.g., all) positions at which the codon pairs of Table 1 give no codon or conflicting or multiple codons.

In some embodiments, where conflicting or multiple codons are given, codons that do not appear in Table 3 are eliminated, i.e., removed from further consideration for inclusion in the ORF. In some embodiments, where conflicting or multiple codons are given, codons that appear in Table 4 are eliminated. In some embodiments, where conflicting or multiple codons are given, codons that would result in the presence of a codon pair in Table 2 are eliminated. These may be combined in any order. For example, first eliminate codons that would result in the presence of a codon pair in Table 2, then if more than one possibility remains, eliminate codons that do not appear in Table 3 and/or codons that appear in Table 4. If any of these approaches eliminate all possible codons, one may proceed as if no codon was given for the position.

In some embodiments, where conflicting or multiple codons are given, the codon that minimizes uridine content is used. In some embodiments, where conflicting or multiple codons are given, the codon that minimizes repeat content is used. In some embodiments, where conflicting or multiple codons are given, the codon that maximizes GC content is used. Any combination of these steps may be applied hierarchically in case a first step does not provide a single codon to be used. For example, first select based on minimization of uridines; then select based on minimization of repeats; then select based on maximization of GC content. The above described steps to narrow down the possible codons for each amino acid are generally sufficient to resolve each position to a single codon; however, if more than one possibility remains, one can be chosen essentially at random, e.g., using a pseudorandom number generator, or by resorting to a one-to-one codon set, such as any of those described herein.

In some embodiments, where conflicting or multiple codons are given, codons that do not appear in Table 3 are eliminated and optionally codons that appear in Table 4 are eliminated and/or codons that would result in the presence of a codon pair in Table 2 are eliminated, and then at least one of the following is applied: the codon that minimizes uridine content is used; the codon that minimizes repeat content is used; and/or the codon that maximizes GC content is used. Any combination of these steps may be applied hierarchically in case a first step does not provide a single codon to be used. For example, first select based on minimization of uridines; then select based on minimization of repeats; then select based on maximization of GC content. The above described steps to narrow down the possible codons for each amino acid are generally sufficient to resolve each position to a single codon; however, if more than one possibility remains, one can be chosen essentially at random, e.g., using a pseudorandom number generator, or by resorting to a one-to-one codon set, such as any of those described herein.

In some embodiments, where conflicting or multiple codons are given, codons that appear in Table 4 are eliminated and optionally codons do not that appear in Table 3 are eliminated and/or codons that would result in the presence of a codon pair in Table 2 are eliminated, and then at least one of the following is applied: the codon that minimizes uridine content is used; the codon that minimizes repeat content is used; and/or the codon that maximizes GC content is used. Any combination of these steps may be applied hierarchically in case a first step does not provide a single codon to be used. For example, first select based on minimization of uridines; then select based on minimization of repeats; then select based on maximization of GC content. The above described steps to narrow down the possible codons for each amino acid are generally sufficient to resolve each position to a single codon; however, if more than one possibility remains, one can be chosen essentially at random, e.g., using a pseudorandom number generator, or by resorting to a one-to-one codon set, such as any of those described herein.

In some embodiments, where conflicting or multiple codons are given, codons that would result in the presence of a codon pair in Table 2 are eliminated and optionally codons do not that appear in Table 3 are eliminated and/or codons that appear in Table 4 are eliminated, and then at least one of the following is applied: the codon that minimizes uridine content is used; the codon that minimizes repeat content is used; and/or the codon that maximizes GC content is used. Any combination of these steps may be applied hierarchically in case a first step does not provide a single codon to be used. For example, first select based on minimization of uridines; then select based on minimization of repeats; then select based on maximization of GC content. The above described steps to narrow down the possible codons for each amino acid are generally sufficient to resolve each position to a single codon; however, if more than one possibility remains, one can be chosen essentially at random, e.g., using a pseudorandom number generator, or by resorting to a one-to-one codon set, such as any of those described herein.

Where no codon was given (and optionally where conflicting or multiple codons are given), one may start from the set of all available codons for the amino acid to be encoded; the set of all available codons for the amino acid to be encoded except those that appear in Table 4; the set of all available codons for the amino acid to be encoded except those that would result in the presence of a codon pair in Table 2; the set of all available codons for the amino acid to be encoded except those that appear in Table 4 or would result in the presence of a codon pair in Table 2; and then apply an approach discussed above or combination thereof, such as to first select based on minimization of uridines; then select based on minimization of repeats; then select based on maximization of GC content. Alternatively, one can simply resort to a one-to-one codon set, such as any of those described herein. Exemplary codon sets appear in the following tables. These sets may also be used to implement the third option set forth above, i.e., to use a codon set that provides a specific codon for each amino acid whenever selection of codon pairs from Table 1 does not provide a single codon at a given position.

TABLE 5
Codons correlated with long mRNA half-life

| Amino Acid | Codon |
|---|---|
| Gly | GGT |
| Glu | GAA |
| Asp | GAC |
| Val | GTC |
| Ala | GCC |
| Arg | AGA |
| Ser | TCT |
| Lys | AAG |
| Asn | AAC |
| Met | ATG |
| Ile | ATC |
| Thr | ACC |
| Trp | TGG |
| Cys | TGC |
| Tyr | TAC |
| Leu | TTG |
| Phe | TTC |
| Gln | CAA |
| His | CAC |

TABLE 6
Codons correlated with high liver expression
and minimal uridine content

| Amino Acid | Codon |
|---|---|
| Gly | GGC |
| Glu | GAG |
| Asp | GAC |
| Val | GTG |
| Ala | GCC |
| Arg | AGA |
| Ser | AGC |
| Lys | AAG |
| Asn | AAC |
| Met | ATG |
| Ile | ATC |
| Thr | ACC |
| Trp | TGG |
| Cys | TGC |
| Tyr | TAC |
| Leu | CTG |
| Phe | TTC |
| Gln | CAG |
| His | CAC |

TABLE 7
Additional exemplary codon sets.

| Amino | | | | | | Low |
|---|---|---|---|---|---|---|
| Acid | Low U | High U | Low G | Low C | Low A | A/U |
| Gly | GGC | GGT | GGC | GGA | GGC | GGC |
| Glu | GAG | GAA | GAA | GAG | GAG | GAG |
| Asp | GAC | GAT | GAC | GAT | GAC | GAC |
| Val | GTG | GTT | GTC | GTG | GTG | GTG |
| Ala | GCC | GCT | GCC | GCT | GCC | GCC |
| Arg | AGA | CGT | AGA | AGA | CGG | CGG |
| Ser | AGC | TCT | TCC | AGT | TCC | AGC |
| Lys | AAG | AAA | AAA | AAG | AAG | AAG |
| Asn | AAC | AAT | AAC | AAT | AAC | AAC |
| Met | ATG | ATG | ATG | AGT | ATG | ATG |
| Ile | ATC | ATT | ATC | ATT | ATC | ATC |
| Thr | ACC | ACT | ACC | ACA | ACC | ACC |
| Trp | TGG | TGG | TGG | TGG | TGG | TGG |
| Cys | TGC | TGT | TGC | TGT | TGC | TGC |
| Tyr | TAC | TAT | TAC | TAT | TAC | TAC |
| Leu | CTG | TTA | CTC | TTG | CTG | CTG |
| Phe | TTC | TTT | TTC | TTT | TTC | TTC |
| Gln | CAG | CAA | CAA | CAG | CAG | CAG |
| His | CAC | CAT | CAC | CAT | CAC | CAC |

Where a set from Table 7 is used, in some embodiments, the set is the low U, low A, or low A/U set.

Exemplary ORF sequences that encode a Cas9 nuclease and are enriched or depleted for different sets of codons and codon pairs are provided herein as SEQ ID NOs: 5-14. generated according to the method disclosed herein. The set of ORF sequences provide different enrichments or depletions in codon pairs, as shown in Table 8.

TABLE 8
Characteristics of Exemplary ORF Sequences.

| | | Count | | | | Percentage | | | | Repeat | GC |
|---|---|---|---|---|---|---|---|---|---|---|---|
| SEQ | | I- | E- | I- | E- | I- | E- | I- | E- | content | content |
| ID NO | Brief description | pairs | pairs | singles | singles | pairs | pairs | singles | singles | (%) | (%) |
| 5 | E-Single enriched | 0 | 8 | 0 | 1196 | 0.000 | 0.581 | 0.000 | 86.792 | 23.5 | 61.8 |
| 6 | E-pair enriched, I-pair depleted | 9 | 51 | 87 | 1099 | 0.653 | 3.701 | 6.313 | 79.753 | 22.7 | 59.5 |
| | (optionally either E-single enriched | | | | | | | | | | |
| | or I-single depleted) | | | | | | | | | | |
| 7 | E-pair & E-single enriched, I-pair | 6 | 85 | 97 | 1086 | 0.435 | 6.168 | 7.039 | 78.810 | 22.4 | 59.0 |
| | & I-single depleted | | | | | | | | | | |
| 8 | I-pair depleted and/or I-single | 8 | 87 | 102 | 1081 | 0.581 | 6.313 | 7.402 | 78.447 | 23.0 | 59.1 |
| | depleted | | | | | | | | | | |
| 9 | E-Pair enriched | 8 | 87 | 102 | 1081 | 0.581 | 6.313 | 7.402 | 78.447 | 22.3 | 59.0 |
| 10 | E-pair and E-single enriched | 0 | 26 | 0 | 1196 | 0.000 | 1.887 | 0.000 | 86.792 | 22.7 | 61.8 |
| 11 | E-single depleted | 0 | 19 | 0 | 1041 | 0.000 | 1.379 | 0.000 | 75.544 | 27.5 | 52.1 |
| 12 | I-single enriched | 0 | 8 | 0 | 1196 | 0.000 | 0.581 | 0.000 | 86.792 | 27.2 | 58.1 |
| 13 | E-pair depleted | 19 | 4 | 1024 | 95 | 1.379 | 0.290 | 74.311 | 6.894 | 34.1 | 23.8 |
| 14 | I-pair enriched | 13 | 85 | 809 | 328 | 0.943 | 6.168 | 58.708 | 23.803 | 31.9 | 32.0 |
| 29 | E-pair enriched; Table 6 codon | 10 | 85 | 338 | 845 | 0.725 | 6.159 | 24.493 | 61.232 | 22.7 | 51.9 |
| | enriched | | | | | | | | | | |
| 46 | E-pair enriched; Table 7 Low A | 6 | 85 | 97 | 1040 | 0.435 | 6.159 | 7.029 | 75.362 | 25.2 | 59.1 |
| | codon enriched | | | | | | | | | | |

In Table 8, E-pairs, I-pairs, E-singles, and I-singles refer, respectively, to the codon pairs or codons of Tables 1-4. In addition to the enrichments or depletions shown in the brief description column, all of SEQ ID NOs: 5-10 were further subjected to steps of minimizing uridines, minimizing repeats, and maximizing GC content. SEQ ID NOs: 29 and 46 used codons of Table 6 and the Low A set of Table 7, respectively, at positions where codon pairs of Table 1 were not used. Enrichments or depletions shown in parentheses were dispensable in that they did not further modify the sequences compared to sequences generated with the enrichment/depletion steps not in parentheses plus the steps of minimizing uridines, minimizing repeats, and maximizing GC content. In addition to the enrichments or depletions shown in the brief description column, all of SEQ ID NOs: 11-14 were further subjected to steps of maximizing uridines, maximizing repeats, and minimizing GC content. In all cases, enrichment/depletion steps (where used) were performed in the following order: E-pairs; I-pairs; E-singles; I-singles; uridines; repeats; GC content. Once a given position has converged to a single codon without conflicts due to overlapping pairs, no further steps are applied for that position.

In any of the embodiments set forth herein, the polynucleotide comprising an open reading frame (ORF) encoding a polypeptide may be an mRNA. In any of the embodiments set forth herein, the polynucleotide comprising an open reading frame (ORF) encoding a polypeptide may be an expression construct comprising a promoter operably linked to the ORF.

### 2. ORFs with Low Uridine Content

In some embodiments, the ORF encoding a polypeptide has a uridine content ranging from its minimum uridine content to about 150% of its minimum uridine content. In some embodiments, the uridine content of the ORF is less than or equal to about 145%, 140%, 135%, 130%, 125%, 120%, 115%, 110%, 105%, 104%, 103%, 102%, or 101% of its minimum uridine content. In some embodiments, the ORF has a uridine content equal to its minimum uridine content. In some embodiments, the ORF has having a uridine content less than or equal to about 150% of its minimum uridine content. In some embodiments, the ORF has a uridine content less than or equal to about 145% of its minimum uridine content. In some embodiments, the ORF has a uridine content less than or equal to about 140% of its minimum uridine content. In some embodiments, the ORF has a uridine content less than or equal to about 135% of its minimum uridine content. In some embodiments, the ORF has a uridine content less than or equal to about 130% of its minimum uridine content. In some embodiments, the ORF has a uridine content less than or equal to about 125% of its minimum uridine content. In some embodiments, the ORF has a uridine content less than or equal to about 120% of its minimum uridine content. In some embodiments, the ORF has a uridine content less than or equal to about 115% of its minimum uridine content. In some embodiments, the ORF has a uridine content less than or equal to about 110% of its minimum uridine content. In some embodiments, the ORF has a uridine content less than or equal to about 105% of its minimum uridine content. In some embodiments, the ORF has a uridine content less than or equal to about 104% of its minimum uridine content. In some embodiments, the ORF has a uridine content less than or equal to about 103% of its minimum uridine content. In some embodiments, the ORF has a uridine content less than or equal to about 102% of its minimum uridine content. In some embodiments, the ORF has a uridine content less than or equal to about 101% of its minimum uridine content.

In some embodiments, the ORF has a uridine dinucleotide content ranging from its minimum uridine dinucleotide content to 200% of its minimum uridine dinucleotide content. In some embodiments, the uridine dinucleotide content of the ORF is less than or equal to about 195%, 190%, 185%, 180%, 175%, 170%, 165%, 160%, 155%, 150%, 145%, 140%, 135%, 130%, 125%, 120%, 115%, 110%, 105%, 104%, 103%, 102%, or 101% of its minimum uridine dinucleotide content. In some embodiments, the ORF has a uridine dinucleotide content equal to its minimum uridine dinucleotide content. In some embodiments, the ORF has a uridine dinucleotide content less than or equal to about 200% of its minimum uridine dinucleotide content. In some embodiments, the ORF has a uridine dinucleotide content less than or equal to about 195% of its minimum uridine dinucleotide content. In some embodiments, the ORF has a uridine dinucleotide content less than or equal to about 190% of its minimum uridine dinucleotide content. In some embodiments, the ORF has a uridine dinucleotide content less than or equal to about 185% of its minimum uridine dinucleotide content. In some embodiments, the ORF has a uridine dinucleotide content less than or equal to about 180% of its minimum uridine dinucleotide content. In some embodiments, the ORF has a uridine dinucleotide content less than or equal to about 175% of its minimum uridine dinucleotide content. In some embodiments, the ORF has a uridine dinucleotide content less than or equal to about 170% of its minimum uridine dinucleotide content. In some embodiments, the ORF has a uridine dinucleotide content less than or equal to about 165% of its minimum uridine dinucleotide content. In some embodiments, the ORF has a uridine dinucleotide content less than or equal to about 160% of its minimum uridine dinucleotide content. In some embodiments, the ORF has a uridine dinucleotide content less than or equal to about 155% of its minimum uridine dinucleotide content. In some embodiments, the ORF has a uridine dinucleotide content equal to its minimum uridine dinucleotide content. In some embodiments, the ORF has a uridine dinucleotide content less than or equal to about 150% of its minimum uridine dinucleotide content. In some embodiments, the ORF has a uridine dinucleotide content less than or equal to about 145% of its minimum uridine dinucleotide content. In some embodiments, the ORF has a uridine dinucleotide content less than or equal to about 140% of its minimum uridine dinucleotide content. In some embodiments, the ORF has a uridine dinucleotide content less than or equal to about 135% of its minimum uridine dinucleotide content. In some embodiments, the ORF has a uridine dinucleotide content less than or equal to about 130% of its minimum uridine dinucleotide content. In some embodiments, the ORF has a uridine dinucleotide content less than or equal to about 125% of its minimum uridine dinucleotide content. In some embodiments, the ORF has a uridine dinucleotide content less than or equal to about 120% of its minimum uridine dinucleotide content. In some embodiments, the ORF has a uridine dinucleotide content less than or equal to about 115% of its minimum uridine dinucleotide content. In some embodiments, the ORF has a uridine dinucleotide content less than or equal to about 110% of its minimum uridine dinucleotide content. In some embodiments, the ORF has a uridine dinucleotide content less than or equal to about 105% of its minimum uridine dinucleotide content. In some embodiments, the ORF has a uridine dinucleotide content less than or equal to about 104% of its minimum uridine dinucleotide content. In some embodiments, the ORF has a uridine dinucleotide content less than or equal to about 103% of its minimum uridine dinucleotide content. In some embodiments, the ORF has a uridine dinucleotide content less than or equal to about 102% of its minimum uridine dinucleotide content. In some embodiments, the ORF has a uridine dinucleotide content less than or equal to about 101% of its minimum uridine dinucleotide content.

In some embodiments, the ORF has a uridine dinucleotide content ranging from its minimum uridine dinucleotide content to the uridine dinucleotide content that is 90% or lower of the maximum uridine dinucleotide content of a reference sequence that encodes the same protein as the mRNA in question. In some embodiments, the uridine dinucleotide content of the ORF is less than or equal to about 85%, 80%, 75%, 70%, 65%, 60%, 55%, 50%, 45%, 40%, 35%, 30%, 25%, 20%, 15%, 10%, or 5% of the maximum uridine dinucleotide content of a reference sequence that encodes the same protein as the mRNA in question.

In some embodiments, the ORF has a uridine trinucleotide content ranging from 0 uridine trinucleotides to 1, 2, 3, 4, 5, 6, 7, 8, 9, 10, 20, 30, 40, or 50 uridine trinucleotides (where a longer run of uridines counts as the number of unique three-uridine segments within it, e.g., a uridine tetranucleotide contains two uridine trinucleotides, a uridine pentanucleotide contains three uridine trinucleotides, etc.). In some embodiments, the ORF has a uridine trinucleotide content ranging from 0% uridine trinucleotides to 0.1%, 0.2%, 0.3%, 0.4%, 0.5%, 0.6%, 0.7%, 0.8%, 0.9%, 1%, 1.5%, or 2% uridine trinucleotides, where the percentage content of uridine trinucleotides is calculated as the percentage of positions in a sequence that are occupied by uridines that form part of a uridine trinucleotide (or longer run of uridines), such that the sequences UUUAAA and UUUUAAAA would each have a uridine trinucleotide content of 50%. For example, in some embodiments, the ORF has a uridine trinucleotide content less than or equal to 2%. For example, in some embodiments, the ORF has a uridine trinucleotide content less than or equal to 1.5%. In some embodiments, the ORF has a uridine trinucleotide content less than or equal to 1%. In some embodiments, the ORF has a uridine trinucleotide content less than or equal to 0.9%. In some embodiments, the ORF has a uridine trinucleotide content less than or equal to 0.8%. In some embodiments, the ORF has a uridine trinucleotide content less than or equal to 0.7%. In some embodiments, the ORF has a uridine trinucleotide content less than or equal to 0.6%. In some embodiments, the ORF has a uridine trinucleotide content less than or equal to 0.5%. In some embodiments, the ORF has a uridine trinucleotide content less than or equal to 0.4%. In some embodiments, the ORF has a uridine trinucleotide content less than or equal to 0.3%. In some embodiments, the ORF has a uridine trinucleotide content less than or equal to 0.2%. In some embodiments, the ORF has a uridine trinucleotide content less than or equal to 0.1%. In some embodiments, the ORF has no uridine trinucleotides.

In some embodiments, the ORF has a uridine trinucleotide content ranging from its minimum uridine trinucleotide content to the uridine trinucleotide content that is 90% or lower of the maximum uridine trinucleotide content of a reference sequence that encodes the same protein as the polynucleotide in question. In some embodiments, the uridine trinucleotide content of the ORF is less than or equal to about 85%, 80%, 75%, 70%, 65%, 60%, 55%, 50%, 45%, 40%, 35%, 30%, 25%, 20%, 15%, 10%, or 5% of the maximum uridine trinucleotide content of a reference sequence that encodes the same protein as the polynucleotide in question.

In some embodiments, the ORF has minimal nucleotide homopolymers, e.g., repetitive strings of the same nucleotides. For example, in some embodiments, when selecting a minimal uridine codon from the codons listed in Table 9, a polynucleotide is constructed by selecting the minimal uridine codons that reduce the number and length of nucleotide homopolymers, e.g., selecting GCA instead of GCC for alanine or selecting GGA instead of GGG for glycine or selecting AAG instead of AAA for lysine.

A given ORF can be reduced in uridine content or uridine dinucleotide content or uridine trinucleotide content, for example, by using minimal uridine codons in a sufficient fraction of the ORF. For example, an amino acid sequence for a polypeptide encoded by the ORF described herein can be back-translated into an ORF sequence by converting amino acids to codons, wherein some or all of the ORF uses the exemplary minimal uridine codons shown below. In some embodiments, at least about 50%, 55%, 60%, 65%, 70%, 75%, 80%, 85%, 90%, 95%, 98%, 99%, or 100% of the codons in the ORF are codons listed in Table 9.

TABLE 9
Exemplary minimal uridine codons

| | Amino Acid | Minimal uridine codon |
|---|---|---|
| A | Alanine | GCA or GCC or GCG |
| G | Glycine | GGA or GGC or GGG |
| V | Valine | GUC or GUA or GUG |
| D | Aspartic acid | GAC |
| E | Glutamic acid | GAA or GAG |
| I | Isoleucine | AUC or AUA |
| T | Threonine | ACA or ACC or ACG |
| N | Asparagine | AAC |
| K | Lysine | AAG or AAA |
| S | Serine | AGC |
| R | Arginine | AGA or AGG |
| L | Leucine | CUG or CUA or CUC |
| P | Proline | CCG or CCA or CCC |
| H | Histidine | CAC |
| Q | Glutamine | CAG or CAA |
| F | Phenylalanine | UUC |
| Y | Tyrosine | UAC |
| C | Cysteine | UGC |
| W | Tryptophan | UGG |
| M | Methionine | AUG |

In some embodiments, the ORF consists of a set of codons of which at least about 75%, 80%, 85%, 90%, 95%, 98%, 99%, or 100% of the codons are codons listed in Table 9.

### 3. ORFs with Low Adenine Content

In some embodiments, the ORF has an adenine content ranging from its minimum adenine content to about 150% of its minimum adenine content. In some embodiments, the adenine content of the ORF is less than or equal to about 145%, 140%, 135%, 130%, 125%, 120%, 115%, 110%, 105%, 104%, 103%, 102%, or 101% of its minimum adenine content. In some embodiments, the ORF has an adenine content equal to its minimum adenine content. In some embodiments, the ORF has an adenine content less than or equal to about 150% of its minimum adenine content. In some embodiments, the ORF has an adenine content less than or equal to about 145% of its minimum adenine content. In some embodiments, the ORF has an adenine content less than or equal to about 140% of its minimum adenine content. In some embodiments, the ORF has an adenine content less than or equal to about 135% of its minimum adenine content. In some embodiments, the ORF has an adenine content less than or equal to about 130% of its minimum adenine content. In some embodiments, the ORF has an adenine content less than or equal to about 125% of its minimum adenine content. In some embodiments, the ORF has an adenine content less than or equal to about 120% of its minimum adenine content. In some embodiments, the ORF has an adenine content less than or equal to about 115% of its minimum adenine content. In some embodiments, the ORF has an adenine content less than or equal to about 110% of its minimum adenine content. In some embodiments the ORF has an adenine content less than or equal to about 105% of its minimum adenine content. In some embodiments, the ORF has an adenine content less than or equal to about 104% of its minimum adenine content. In some embodiments, the ORF has an adenine content less than or equal to about 103% of its minimum adenine content. In some embodiments, the ORF has an adenine content less than or equal to about 102% of its minimum adenine content. In some embodiments, the ORF has an adenine content less than or equal to about 101% of its minimum adenine content.

In some embodiments, the ORF has an adenine dinucleotide content ranging from its minimum adenine dinucleotide content to 200% of its minimum adenine dinucleotide content. In some embodiments, the adenine dinucleotide content of the ORF is less than or equal to about 195%, 190%, 185%, 180%, 175%, 170%, 165%, 160%, 155%, 150%, 145%, 140%, 135%, 130%, 125%, 120%, 115%, 110%, 105%, 104%, 103%, 102%, or 101% of its minimum adenine dinucleotide content. In some embodiments, the ORF has an adenine dinucleotide content equal to its minimum adenine dinucleotide content. In some embodiments, the ORF has an adenine dinucleotide content less than or equal to about 200% of its minimum adenine dinucleotide content. In some embodiments, the ORF has an adenine dinucleotide content less than or equal to about 195% of its minimum adenine dinucleotide content. In some embodiments, the ORF has an adenine dinucleotide content less than or equal to about 190% of its minimum adenine dinucleotide content. In some embodiments, the ORF has an adenine dinucleotide content less than or equal to about 185% of its minimum adenine dinucleotide content. In some embodiments, the ORF has an adenine dinucleotide content less than or equal to about 180% of its minimum adenine dinucleotide content. In some embodiments, the ORF has an adenine dinucleotide content less than or equal to about 175% of its minimum adenine dinucleotide content. In some embodiments, the ORF has an adenine dinucleotide content less than or equal to about 170% of its minimum adenine dinucleotide content. In some embodiments, the ORF has an adenine dinucleotide content less than or equal to about 165% of its minimum adenine dinucleotide content. In some embodiments, the ORF has an adenine dinucleotide content less than or equal to about 160% of its minimum adenine dinucleotide content. In some embodiments, the ORF has an adenine dinucleotide content less than or equal to about 155% of its minimum adenine dinucleotide content. In some embodiments, the ORF has an adenine dinucleotide content equal to its minimum adenine dinucleotide content. In some embodiments, the ORF has an adenine dinucleotide content less than or equal to about 150% of its minimum adenine dinucleotide content. In some embodiments, the ORF has an adenine dinucleotide content less than or equal to about 145% of its minimum adenine dinucleotide content. In some embodiments, the ORF has an adenine dinucleotide content less than or equal to about 140% of its minimum adenine dinucleotide content. In some embodiments, the ORF has an adenine dinucleotide content less than or equal to about 135% of its minimum adenine dinucleotide content. In some embodiments, the ORF has an adenine dinucleotide content less than or equal to about 130% of its minimum adenine dinucleotide content. In some embodiments, the ORF has an adenine dinucleotide content less than or equal to about 125% of its minimum adenine dinucleotide content. In some embodiments, the ORF has an adenine dinucleotide content less than or equal to about 120% of its minimum adenine dinucleotide content. In some embodiments, the ORF has an adenine dinucleotide content less than or equal to about 115% of its minimum adenine dinucleotide content. In some embodiments, the ORF has an adenine dinucleotide content less than or equal to about 110% of its minimum adenine dinucleotide content. In some embodiments, the ORF has an adenine dinucleotide content less than or equal to about 105% of its minimum adenine dinucleotide content. In some embodiments, the ORF has an adenine dinucleotide content less than or equal to about 104% of its minimum adenine dinucleotide content. In some embodiments, the ORF has an adenine dinucleotide content less than or equal to about 103% of its minimum adenine dinucleotide content. In some embodiments, the ORF has an adenine dinucleotide content less than or equal to about 102% of its minimum adenine dinucleotide content. In some embodiments, the ORF has an adenine dinucleotide content less than or equal to about 101% of its minimum adenine dinucleotide content.

In some embodiments, the ORF has an adenine dinucleotide content ranging from its minimum adenine dinucleotide content to the adenine dinucleotide content that is 90% or lower of the maximum adenine dinucleotide content of a reference sequence that encodes the same protein as the polynucleotide in question. In some embodiments, the adenine dinucleotide content of the ORF is less than or equal to about 85%, 80%, 75%, 70%, 65%, 60%, 55%, 50%, 45%, 40%, 35%, 30%, 25%, 20%, 15%, 10%, or 5% of the maximum adenine dinucleotide content of a reference sequence that encodes the same protein as the polynucleotide in question.

In some embodiments, the ORF has an adenine trinucleotide content ranging from 0 adenine trinucleotides to 1, 2, 3, 4, 5, 6, 7, 8, 9, 10, 20, 30, 40, or 50 adenine trinucleotides (where a longer run of adenines counts as the number of unique three-adenine segments within it, e.g., an adenine tetranucleotide contains two adenine trinucleotides, an adenine pentanucleotide contains three adenine trinucleotides, etc.). In some embodiments, the ORF has an adenine trinucleotide content ranging from 0% adenine trinucleotides to 0.1%, 0.2%, 0.3%, 0.4%, 0.5%, 0.6%, 0.7%, 0.8%, 0.9%, 1%, 1.5%, or 2% adenine trinucleotides, where the percentage content of adenine trinucleotides is calculated as the percentage of positions in a sequence that are occupied by adenines that form part of an adenine trinucleotide (or longer run of adenines), such that the sequences UUUAAA and UUUUAAAA would each have an adenine trinucleotide content of 50%. For example, in some embodiments, the ORF has an adenine trinucleotide content less than or equal to 2%. For example, in some embodiments, the ORF has an adenine trinucleotide content less than or equal to 1.5%. In some embodiments, the ORF has an adenine trinucleotide content less than or equal to 1%. In some embodiments, the ORF has an adenine trinucleotide content less than or equal to 0.9%. In some embodiments, the ORF has an adenine trinucleotide content less than or equal to 0.8%. In some embodiments, the ORF has an adenine trinucleotide content less than or equal to 0.7%. In some embodiments, the ORF has an adenine trinucleotide content less than or equal to 0.6%. In some embodiments, the ORF has an adenine trinucleotide content less than or equal to 0.5%. In some embodiments, the ORF has an adenine trinucleotide content less than or equal to 0.4%. In some embodiments, the ORF has an adenine trinucleotide content less than or equal to 0.3%. In some embodiments, the ORF has an adenine trinucleotide content less than or equal to 0.2%. In some embodiments, the ORF has an adenine trinucleotide content less than or equal to 0.1%. In some embodiments, the ORF has no adenine trinucleotides.

In some embodiments, the ORF has an adenine trinucleotide content ranging from its minimum adenine trinucleotide content to the adenine trinucleotide content that is 90% or lower of the maximum adenine trinucleotide content of a reference sequence that encodes the same protein as the polynucleotide in question. In some embodiments, the adenine trinucleotide content of the ORF is less than or equal to about 85%, 80%, 75%, 70%, 65%, 60%, 55%, 50%, 45%, 40%, 35%, 30%, 25%, 20%, 15%, 10%, or 5% of the maximum adenine trinucleotide content of a reference sequence that encodes the same protein as the polynucleotide in question. In some embodiments, the ORF has minimal nucleotide homopolymers, e.g., repetitive strings of the same nucleotides. For example, in some embodiments, when selecting a minimal adenine codon from the codons listed in Table 10, a polynucleotide is constructed by selecting the minimal adenine codons that reduce the number and length of nucleotide homopolymers, e.g., selecting GCA instead of GCC for alanine or selecting GGA instead of GGG for glycine or selecting AAG instead of AAA for lysine. A given ORF can be reduced in adenine content or adenine dinucleotide content or adenine trinucleotide content, for example, by using minimal adenine codons in a sufficient fraction of the ORF. For example, an amino acid sequence for a polypeptide encoded by the ORF described herein can be back-translated into an ORF sequence by converting amino acids to codons, wherein some or all of the ORF uses the exemplary minimal adenine codons shown below. In some embodiments, at least about 50%, 55%, 60%, 65%, 70%, 75%, 80%, 85%, 90%, 95%, 98%, 99%, or 100% of the codons in the ORF are codons listed in Table 10.

TABLE 10
Exemplary minimal adenine codons

| | Amino Acid | Minimal adenine codon |
|---|---|---|
| A | Alanine | GCU or GCC or GCG |
| G | Glycine | GGU or GGC or GGG |
| V | Valine | GUC or GUU or GUG |
| D | Aspartic acid | GAC or GAU |
| E | Glutamic acid | GAG |
| I | Isoleucine | AUC or AUU |
| T | Threonine | ACU or ACC or ACG |
| N | Asparagine | AAC or AAU |
| K | Lysine | AAG |
| S | Serine | UCU or UCC or UCG |
| R | Arginine | CGU or CGC or CGG |
| L | Leucine | CUG or CUC or CUU |
| P | Proline | CCG or CCU or CCC |
| H | Histidine | CAC or CAU |
| Q | Glutamine | CAG |
| F | Phenylalanine | UUC or UUU |
| Y | Tyrosine | UAC or UAU |
| C | Cysteine | UGC or UGU |
| W | Tryptophan | UGG |
| M | Methionine | AUG |

In some embodiments, the ORF consists of a set of codons of which at least about 75%, 80%, 85%, 90%, 95%, 98%, 99%, or 100% of the codons are codons listed in Table 10.

### 4. ORFs with Low Adenine and Low Uridine Content

To the extent feasible, any of the features described above with respect to low adenine content can be combined with any of the features described above with respect to low uridine content. For example, the ORF has a uridine content ranging from its minimum uridine content to about 150% of its minimum uridine content (e.g., a uridine content of the ORF is less than or equal to about 145%, 140%, 135%, 130%, 125%, 120%, 115%, 110%, 105%, 104%, 103%, 102%, or 101% of its minimum uridine content) and an adenine content ranging from its minimum adenine content to about 150% of its minimum adenine content (e.g., less than or equal to about 145%, 140%, 135%, 130%, 125%, 120%, 115%, 110%, 105%, 104%, 103%, 102%, or 101% of its minimum adenine content). So too for uridine and adenine dinucleotides. Similarly, the content of uridine nucleotides and adenine dinucleotides in the ORF may be as set forth above. Similarly, the content of uridine dinucleotides and adenine nucleotides in the ORF may be as set forth above.

A given ORF can be reduced in uridine and adenine nucleotide and/or dinucleotide content, for example, by using minimal uridine and adenine codons in a sufficient fraction of the ORF. For example, an amino acid sequence for a polypeptide encoded by the ORF described herein can be back-translated into an ORF sequence by converting amino acids to codons, wherein some or all of the ORF uses the exemplary minimal uridine and adenine codons shown below. In some embodiments, at least about 50%, 55%, 60%, 65%, 70%, 75%, 80%, 85%, 90%, 95%, 98%, 99%, or 100% of the codons in the ORF are codons listed in Table 11.

TABLE 11
Exemplary minimal uridine and adenine codons

| | Amino Acid | Minimal uridine codon |
|---|---|---|
| A | Alanine | GCC or GCG |
| G | Glycine | GGC or GGG |
| V | Valine | GUC or GUG |
| D | Aspartic acid | GAC |
| E | Glutamic acid | GAG |
| I | Isoleucine | AUC |
| T | Threonine | ACC or ACG |
| N | Asparagine | AAC |
| K | Lysine | AAG |
| S | Serine | AGC or UCC or UCG |
| R | Arginine | CGC or CGG |
| L | Leucine | CUG or CUC |
| P | Proline | CCG or CCC |
| H | Histidine | CAC |
| Q | Glutamine | CAG |
| F | Phenylalanine | UUC |
| Y | Tyrosine | UAC |
| C | Cysteine | UGC |
| W | Tryptophan | UGG |
| M | Methionine | AUG |

In some embodiments, the ORF consists of a set of codons of which at least about 75%, 80%, 85%, 90%, 95%, 98%, 99%, or 100% of the codons are codons listed in Table 11. As can be seen in Table 11, each of the three listed serine codons contains either one A or one U. In some embodiments, uridine minimization is prioritized by using AGC codons for serine. In some embodiments, adenine minimization is prioritized by using UCC and/or UCG codons for serine.

### 5. Codons that Increase Translation and/or that Correspond to Highly Expressed tRNAs; Exemplary Codon Sets

In some embodiments, the ORF has codons that increase translation in a mammal, such as a human. In further embodiments, the ORF has codons that increase translation in an organ, such as the liver, of the mammal, e.g., a human. In further embodiments, the ORF has codons that increase translation in a cell type, such as a hepatocyte, of the mammal, e.g., a human. An increase in translation in a mammal, cell type, organ of a mammal, human, organ of a human, etc., can be determined relative to the extent of translation wild-type sequence of the ORF, or relative to an ORF having a codon distribution matching the codon distribution of the organism from which the ORF was derived or the organism that contains the most similar ORF at the amino acid level.

In some embodiments, the polypeptide encoded by the ORF is a Cas9 nuclease derived from prokaryotes described below, and an increase in translation in a mammal, cell type, organ of a mammal, human, organ of a human, etc., can be determined relative to the extent of translation wild-type sequence of the ORF (e.g., a wild-type ORF listed in the sequence table, such as SEQ ID NO: 67 (Cas9), 68 (SerpinA1), 89 (FAH), 95 (GABRD), 101 (GAPDH), 107 (GBA1), 113 (GLA), 119 (OTC), 125 (PAH), or 131 (TTR), or relative to an ORF of interest, such as an ORF encoding a human protein or transgene for expression in a human cell. For example, the ORF may be an ORF having a codon distribution matching the codon distribution of the organism from which the ORF was derived or the organism that contains the most similar ORF at the amino acid level, such as S. pyogenes, S. aureus, or another prokaryote for Cas proteins, or relative to translation of the Cas9 ORF contained in SEQ ID NO: 2, 3, or 67 with all else equal, including any applicable point mutations, heterologous domains, and the like. Codons useful for increasing expression in a human, including the human liver and human hepatocytes, can be codons corresponding to highly expressed tRNAs in the human liver/hepatocytes, which are discussed in Dittmar K A, PLos Genetics 2(12): e221 (2006). In some embodiments, at least about 75%, 80%, 85%, 90%, 95%, 96%, 97%, 98%, 99%, or 100% of the codons in an ORF are codons corresponding to highly expressed tRNAs (e.g., the highest-expressed tRNA for each amino acid) in a mammal, such as a human. In some embodiments, at least 75%, 80%, 85%, 90%, 95%, 96%, 97%, 98%, 99%, or 100% of the codons in an ORF are codons corresponding to highly expressed tRNAs (e.g., the highest-expressed tRNA for each amino acid) in a mammalian organ, such as a human organ. In some embodiments, at least 75%, 80%, 85%, 90%, 95%, 96%, 97%, 98%, 99%, or 100% of the codons in an ORF are codons corresponding to highly expressed tRNAs (e.g., the highest-expressed tRNA for each amino acid) in a mammalian liver, such as a human liver. In some embodiments, at least 75%, 80%, 85%, 90%, 95%, 96%, 97%, 98%, 99%, or 100% of the codons in an ORF are codons corresponding to highly expressed tRNAs (e.g., the highest-expressed tRNA for each amino acid) in a mammalian hepatocyte, such as a human hepatocyte.

Alternatively, codons corresponding to highly expressed tRNAs in an organism (e.g., human) in general may be used.

Any of the foregoing approaches to codon selection can be combined with selecting codon pairs as shown in Table 1; and/or eliminating codons that appear in Table 4, that would result in the presence of a codon pair shown in Table 2, and/or that would contribute to higher repeat content; and/or selecting codon that appears in Table 3 and/or that contribute to lower repeat content; and/or using a codon set of Table 5, 6, or 7, as shown above; using the minimal uridine and/or adenine codons shown above, e.g., Table 9, 10, or 11, and then where more than one option is available, using the codon that corresponds to a more highly-expressed tRNA, either in the organism (e.g., human) in general, or in an organ or cell type of interest, such as the liver or hepatocytes (e.g., human liver or human hepatocytes).

### 6. Polypeptide Encoded by the ORF; Exemplary Sequences

In some embodiments, the polynucleotide is a mRNA comprising an ORF encoding a polypeptide of interest.

In some embodiments, the polynucleotide is a mRNA comprising an ORF encoding an RNA-guided DNA binding agent disclosed above.

In some embodiments, the ORF comprises a sequence with at least 90% identity to any one of SEQ ID NOs: 6-10, 29, 46, 69-73, 90-93, 96-99, 102-105, 108-111, 114-117, 120-123, 126-129, or 132-143, optionally wherein identity is determined without regard to the start and stop codons of the ORF. Identity is determined “without regard to the start and stop codons of the ORF” by aligning sequences without the start and stop codons; the start and stop codons generally appear at positions 1 to 3 and N-2 to N (where N is the number of nucleotides in the ORF), respectively; and the start and stop codons are usually ATG (or sometimes GTG) and one of TAA, TGA, and TAG, respectively (where the Ts in the start and stop codons may be substituted by U). In some embodiments, the degree of identity to the sequence of SEQ ID NO: 6-10, 29, 46, 69-73, 90-93, 96-99, 102-105, 108-111, 114-117, 120-123, 126-129, or 132-143 is at least 95%. In some embodiments, the degree of identity to the sequence of SEQ ID NO: 6-10, 29, 46, 69-73, 90-93, 96-99, 102-105, 108-111, 114-117, 120-123, 126-129, or 132-143 is at least 98%. In some embodiments, the degree of identity to the sequence of SEQ ID NO: 6-10, 29, 46, 69-73, 90-93, 96-99, 102-105, 108-111, 114-117, 120-123, 126-129, or 132-143 is at least 99%. In some embodiments, the degree of identity to the sequence of SEQ ID NOs: 6-10, 29, 46, 69-73, 90-93, 96-99, 102-105, 108-111, 114-117, 120-123, 126-129, or 132-143 is 100%.

In some embodiments, the polynucleotide comprises a sequence with at least 90% identity to any one of SEQ ID NOs: 16-20, 76-80, 193-197, or 199-201. In some embodiments, the degree of identity to the sequence of SEQ ID NO: 16-20, 76-80, 193-197, or 199-201 is at least 95%. In some embodiments, the degree of identity to the sequence of SEQ ID NO: 16-20, 76-80, 193-197, or 199-201 is at least 98%. In some embodiments, the degree of identity to the sequence of SEQ ID NO: 16-20, 76-80, 193-197, or 199-201 is at least 99%. In some embodiments, the degree of identity to the sequence of SEQ ID NOs: 16-20, 76-80, 193-197, or 199-201 is 100%. In some embodiments, the polynucleotide comprises a sequence with at least 90% identity to any one of SEQ ID NOs: 16-20, 76-80, 194-197, or 200-201. In some embodiments, the degree of identity to the sequence of SEQ ID NO: 16-20, 76-80, 194-197, or 200-201 is at least 95%. In some embodiments, the degree of identity to the sequence of SEQ ID NO: 16-20, 76-80, 194-197, or 200-201 is at least 98%. In some embodiments, the degree of identity to the sequence of SEQ ID NO: 16-20, 76-80, 194-197, or 200-201 is at least 99%. In some embodiments, the degree of identity to the sequence of SEQ ID NOs: 16-20, 76-80, 194-197, or 200-201 is 100%.

In some embodiments, the polypeptide encoded by the ORF described herein is an RNA-guided DNA binding agent, which is further described below. In some embodiments, the polypeptide encoded by the ORF described herein is an endonuclease. In some embodiments, the polypeptide encoded by the ORF described herein is a serine protease inhibitor or Serpin family member. In some embodiments, the polypeptide encoded by the ORF described herein is a hydroxylase; carbamoyltransferase; glucosylceramidase; galactosidase; dehydrogenase; receptor; or neurotransmitter receptor. In some embodiments, the polypeptide encoded by the ORF described herein is a phenylalanine hydroxylase; an ornithine carbamoyltransferase; a fumarylacetoacetate hydrolase; a glucosylceramidase beta; an alpha galactosidase; a transthyretin; a glyceraldehyde-3-phosphate dehydrogenase; a gamma-aminobutyric acid (GABA) receptor subunit (such as a GABA Type A Receptor Delta Subunit). In some embodiments, the polypeptide encoded by the ORF described herein is a Serpin Family A Member 1.

An exemplary phenylalanine hydroxylase amino acid sequence is SEQ ID NO: 124. Exemplary sequences that encode a phenylalanine hydroxylase are SEQ ID NOs: 126-129 and 142.

An exemplary ornithine carbamoyltransferase amino acid sequence is SEQ ID NO: 118. Exemplary sequences that encode an ornithine carbamoyltransferase are SEQ ID NOs: 120-123 and 141.

An exemplary glucosylceramidase beta amino acid sequence is SEQ ID NO: 106. Exemplary sequences that encode a glucosylceramidase beta are SEQ ID NOs: 108-111 and 139.

An exemplary alpha galactosidase amino acid sequence is SEQ ID NO: 112. Exemplary sequences that encode an alpha galactosidase are SEQ ID NOs: 114-117 and 140.

An exemplary glyceraldehyde-3-phosphate dehydrogenase amino acid sequence is SEQ ID NO: 100. Exemplary sequences that encode a glyceraldehyde-3-phosphate dehydrogenase are SEQ ID NOs: 102-105 and 138.

An exemplary GABA Type A Receptor Delta Subunit amino acid sequence is SEQ ID NO: 94. Exemplary sequences that encode a GABA Type A Receptor Delta Subunit are SEQ ID NOs: 96-99 and 137.

An exemplary fumarylacetoacetate hydrolase amino acid sequence is SEQ ID NO: 88. Exemplary sequences that encode a fumarylacetoacetate hydrolase are SEQ ID NOs: 89-93 and 136.

An exemplary transthyretin amino acid sequence is SEQ ID NO: 130. Exemplary sequences that encode a transthyretin are SEQ ID NOs: 132-135, and 143.

An exemplary Serpin Family A Member 1 amino acid sequence is SEQ ID NO: 74. Exemplary sequences that encode a Serpin Family A Member 1 are SEQ ID NOs: 76-80.

### a) Encoded RNA-Guided DNA Binding Agent

In some embodiments, the polynucleotide encoded by the ORF described herein is an RNA-guided DNA-binding agent. In some embodiments, the RNA-guided DNA-binding agent is a Class 2 Cas nuclease. In some embodiments, the RNA-guided DNA-binding agent has cleavase activity, which can also be referred to as double-strand endonuclease activity. In some embodiments, the RNA-guided DNA-binding agent comprises a Cas nuclease, such as a Class 2 Cas nuclease (which may be, e.g., a Cas nuclease of Type II, V, or VI). Class 2 Cas nucleases include, for example, Cas9, Cpf1, C2c1, C2c2, and C2c3 proteins and modifications thereof. Examples of Cas9 nucleases include those of the type II CRISPR systems of S. pyogenes, S. aureus, and other prokaryotes (see, e.g., the list in the next paragraph), and modified (e.g., engineered or mutant) versions thereof. See, e.g., US2016/0312198 A1; US 2016/0312199 A1. Other examples of Cas nucleases include a Csm or Cmr complex of a type III CRISPR system or the Cas10, Csm1, or Cmr2 subunit thereof; and a Cascade complex of a type I CRISPR system, or the Cas3 subunit thereof. In some embodiments, the Cas nuclease may be from a Type-IIA, Type-IIB, or Type-IIC system. For discussion of various CRISPR systems and Cas nucleases see, e.g., Makarova et al., NAT. REV. MICROBIOL. 9:467-477 (2011); Makarova et al., NAT. REV. MICROBIOL, 13: 722-36 (2015); Shmakov et al., MOLECULAR CELL, 60:385-397 (2015).

Non-limiting exemplary species that the Cas nuclease can be derived from include Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gammaproteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, Acidaminococcus sp., Lachnospiraceae bacterium ND2006, and Acaryochloris marina.

In some embodiments, the Cas nuclease is the Cas9 nuclease from Streptococcus pyogenes. In some embodiments, the Cas nuclease is the Cas9 nuclease from Streptococcus thermophilus. In some embodiments, the Cas nuclease is the Cas9 nuclease from Neisseria meningitidis. In some embodiments, the Cas nuclease is the Cas9 nuclease is from Staphylococcus aureus. In some embodiments, the Cas nuclease is the Cpf1 nuclease from Francisella novicida. In some embodiments, the Cas nuclease is the Cpf1 nuclease from Acidaminococcus sp. In some embodiments, the Cas nuclease is the Cpf1 nuclease from Lachnospiraceae bacterium ND2006. In further embodiments, the Cas nuclease is the Cpf1 nuclease from Francisella tularensis, Lachnospiraceae bacterium, Butyrivibrio proteoclasticus, Peregrinibacteria bacterium, Parcubacteria bacterium, Smithella, Acidaminococcus, candidatus Methanoplasma termitum, Eubacterium eligens, Moraxella bovoculi, Leptospira inadai, Porphyromonas crevioricanis, Prevotella disiens, or Porphyromonas macacae. In certain embodiments, the Cas nuclease is a Cpf1 nuclease from an Acidaminococcus or Lachnospiraceae.

Wild type Cas9 has two nuclease domains: RuvC and HNH. The RuvC domain cleaves the non-target DNA strand, and the HNH domain cleaves the target strand of DNA. In some embodiments, the Cas9 nuclease comprises more than one RuvC domain and/or more than one HNH domain. In some embodiments, the Cas9 nuclease is a wild type Cas9. In some embodiments, the Cas9 is capable of inducing a double strand break in target DNA. In certain embodiments, the Cas nuclease may cleave dsDNA, it may cleave one strand of dsDNA, or it may not have DNA cleavase or nickase activity. An exemplary Cas9 amino acid sequence is provided as SEQ ID NO: 1. Exemplary Cas9 mRNA ORF sequences are provided as SEQ ID NOs: 5-10.

In some embodiments, chimeric Cas nucleases are used, where one domain or region of the protein is replaced by a portion of a different protein. In some embodiments, a Cas nuclease domain may be replaced with a domain from a different nuclease such as FokI. In some embodiments, a Cas nuclease may be a modified nuclease.

In other embodiments, the Cas nuclease may be from a Type-I CRISPR/Cas system. In some embodiments, the Cas nuclease may be a component of the Cascade complex of a Type-I CRISPR/Cas system. In some embodiments, the Cas nuclease may be a Cas3 protein. In some embodiments, the Cas nuclease may be from a Type-III CRISPR/Cas system. In some embodiments, the Cas nuclease may have an RNA cleavage activity.

In some embodiments, the RNA-guided DNA-binding agent has single-strand nickase activity, i.e., can cut one DNA strand to produce a single-strand break, also known as a “nick.” In some embodiments, the RNA-guided DNA-binding agent comprises a Cas nickase. A nickase is an enzyme that creates a nick in dsDNA, i.e., cuts one strand but not the other of the DNA double helix. In some embodiments, a Cas nickase is a version of a Cas nuclease (e.g., a Cas nuclease discussed above) in which an endonucleolytic active site is inactivated, e.g., by one or more alterations (e.g., point mutations) in a catalytic domain. See, e.g., U.S. Pat. No. 8,889,356 for discussion of Cas nickases and exemplary catalytic domain alterations. In some embodiments, a Cas nickase such as a Cas9 nickase has an inactivated RuvC or HNH domain. An exemplary Cas9 nickase amino acid sequence is provided as SEQ ID NO: 161.

In some embodiments, the RNA-guided DNA-binding agent is modified to contain only one functional nuclease domain. For example, the agent protein may be modified such that one of the nuclease domains is mutated or fully or partially deleted to reduce its nucleic acid cleavage activity. In some embodiments, a nickase is used having a RuvC domain with reduced activity. In some embodiments, a nickase is used having an inactive RuvC domain. In some embodiments, a nickase is used having an HNH domain with reduced activity. In some embodiments, a nickase is used having an inactive HNH domain.

In some embodiments, a conserved amino acid within a Cas protein nuclease domain is substituted to reduce or alter nuclease activity. In some embodiments, a Cas nuclease may comprise an amino acid substitution in the RuvC or RuvC-like nuclease domain. Exemplary amino acid substitutions in the RuvC or RuvC-like nuclease domain include D10A (based on the S. pyogenes Cas9 protein). See, e.g., Zetsche et al. (2015) Cell October 22:163(3): 759-771. In some embodiments, the Cas nuclease may comprise an amino acid substitution in the HNH or HNH-like nuclease domain. Exemplary amino acid substitutions in the HNH or HNH-like nuclease domain include E762A, H840A, N863A, H983A, and D986A (based on the S. pyogenes Cas9 protein). See, e.g., Zetsche et al. (2015). Further exemplary amino acid substitutions include D917A, E1006A, and D1255A (based on the Francisella novicida U112 Cpf1 (FnCpf1) sequence (UniProtKB—A0Q7Q2 (CPF1_FRATN)).

In some embodiments, an mRNA encoding a nickase is provided in combination with a pair of guide RNAs that are complementary to the sense and antisense strands of the target sequence, respectively. In this embodiment, the guide RNAs direct the nickase to a target sequence and introduce a DSB by generating a nick on opposite strands of the target sequence (i.e., double nicking). In some embodiments, use of double nicking may improve specificity and reduce off-target effects. In some embodiments, a nickase is used together with two separate guide RNAs targeting opposite strands of DNA to produce a double nick in the target DNA. In some embodiments, a nickase is used together with two separate guide RNAs that are selected to be in close proximity to produce a double nick in the target DNA.

In some embodiments, the RNA-guided DNA-binding agent lacks cleavase and nickase activity. In some embodiments, the RNA-guided DNA-binding agent comprises a dCas DNA-binding polypeptide. A dCas polypeptide has DNA-binding activity while essentially lacking catalytic (cleavase/nickase) activity. In some embodiments, the dCas polypeptide is a dCas9 polypeptide. In some embodiments, the RNA-guided DNA-binding agent lacking cleavase and nickase activity or the dCas DNA-binding polypeptide is a version of a Cas nuclease (e.g., a Cas nuclease discussed above) in which its endonucleolytic active sites are inactivated, e.g., by one or more alterations (e.g., point mutations) in its catalytic domains. See, e.g., US 2014/0186958 A1; US 2015/0166980 A1. An exemplary dCas9 amino acid sequence is provided as SEQ ID NO: 162.

### b) Heterologous Functional Domains; Nuclear Localization Signals

In some embodiments, the RNA-guided DNA-binding agent encoded by the ORF described herein comprises one or more heterologous functional domains (e.g., is or comprises a fusion polypeptide).

In some embodiments, the heterologous functional domain may facilitate transport of the RNA-guided DNA-binding agent into the nucleus of a cell. For example, the heterologous functional domain may be a nuclear localization signal (NLS). In some embodiments, the RNA-guided DNA-binding agent may be fused with 1-10 NLS(s). In some embodiments, the RNA-guided DNA-binding agent may be fused with 1-5 NLS(s). In some embodiments, the RNA-guided DNA-binding agent may be fused with one NLS. Where one NLS is used, the NLS may be linked at the N-terminus or the C-terminus of the RNA-guided DNA-binding agent sequence. In some embodiments, the RNA-guided DNA-binding agent may be fused C-terminally to at least one NLS. An NLS may also be inserted within the RNA-guided DNA binding agent sequence. In other embodiments, the RNA-guided DNA-binding agent may be fused with more than one NLS. In some embodiments, the RNA-guided DNA-binding agent may be fused with 2, 3, 4, or 5 NLSs. In some embodiments, the RNA-guided DNA-binding agent may be fused with two NLSs. In certain circumstances, the two NLSs may be the same (e.g., two SV40 NLSs) or different. In some embodiments, the RNA-guided DNA-binding agent is fused to two SV40 NLS sequences linked at the carboxy terminus. In some embodiments, the RNA-guided DNA-binding agent may be fused with two NLSs, one linked at the N-terminus and one at the C-terminus. In some embodiments, the RNA-guided DNA-binding agent may be fused with 3 NLSs. In some embodiments, the RNA-guided DNA-binding agent may be fused with no NLS. In some embodiments, the NLS may be a monopartite sequence, such as, e.g., the SV40 NLS, PKKKRKV (SEQ ID NO: 163) or PKKKRRV (SEQ ID NO: 175). In some embodiments, the NLS may be a bipartite sequence, such as the NLS of nucleoplasmin, KRPAATKKAGQAKKKK (SEQ ID NO: 176). In some embodiments, the NLS sequence may comprise LAAKRSRTT (SEQ ID NO: 164), QAAKRSRTT (SEQ ID NO: 165), PAPAKRERTT (SEQ ID NO: 166), QAAKRPRTT (SEQ ID NO: 167), RAAKRPRTT (SEQ ID NO: 168), AAAKRSWSMAA (SEQ ID NO: 169), AAAKRVWSMAF (SEQ ID NO: 170), AAAKRSWSMAF (SEQ ID NO: 171), AAAKRKYFAA (SEQ ID NO: 172), RAAKRKAFAA (SEQ ID NO: 173), or RAAKRKYFAV (SEQ ID NO: 174). The NLS may be a snurportin-1 importin-β (IBB domain, e.g. an SPN1-impβ sequence. See Huber et al., 2002, J. Cell Bio., 156, 467-479. In a specific embodiment, a single PKKKRKV (SEQ ID NO: 163) NLS may be linked at the C-terminus of the RNA-guided DNA-binding agent. One or more linkers are optionally included at the fusion site. In some embodiments, one or more NLS(s) according to any of the foregoing embodiments are present in the RNA-guided DNA-binding agent in combination with one or more additional heterologous functional domains, such as any of the heterologous functional domains described below.

In some embodiments, the heterologous functional domain may be capable of modifying the intracellular half-life of the RNA-guided DNA binding agent. In some embodiments, the half-life of the RNA-guided DNA binding agent may be increased. In some embodiments, the half-life of the RNA-guided DNA-binding agent may be reduced. In some embodiments, the heterologous functional domain may be capable of increasing the stability of the RNA-guided DNA-binding agent. In some embodiments, the heterologous functional domain may be capable of reducing the stability of the RNA-guided DNA-binding agent. In some embodiments, the heterologous functional domain may act as a signal peptide for protein degradation. In some embodiments, the protein degradation may be mediated by proteolytic enzymes, such as, for example, proteasomes, lysosomal proteases, or calpain proteases. In some embodiments, the heterologous functional domain may comprise a PEST sequence. In some embodiments, the RNA-guided DNA-binding agent may be modified by addition of ubiquitin or a polyubiquitin chain. In some embodiments, the ubiquitin may be a ubiquitin-like protein (UBL). Non-limiting examples of ubiquitin-like proteins include small ubiquitin-like modifier (SUMO), ubiquitin cross-reactive protein (UCRP, also known as interferon-stimulated gene-15 (ISG15)), ubiquitin-related modifier-1 (URM1), neuronal-precursor-cell-expressed developmentally downregulated protein-8 (NEDD8, also called Rub1 in S. cerevisiae), human leukocyte antigen F-associated (FAT10), autophagy-8 (ATG8) and -12 (ATG12), Fau ubiquitin-like protein (FUB1), membrane-anchored UBL (MUB), ubiquitin fold-modifier-1 (UFM1), and ubiquitin-like protein-5 (UBLS).

In some embodiments, the heterologous functional domain may be a marker domain. Non-limiting examples of marker domains include fluorescent proteins, purification tags, epitope tags, and reporter gene sequences. In some embodiments, the marker domain may be a fluorescent protein. Non-limiting examples of suitable fluorescent proteins include green fluorescent proteins (e.g., GFP, GFP-2, tagGFP, turboGFP, sfGFP, EGFP, Emerald, Azami Green, Monomeric Azami Green, CopGFP, AceGFP, ZsGreen1), yellow fluorescent proteins (e.g., YFP, EYFP, Citrine, Venus, YPet, PhiYFP, ZsYellow1), blue fluorescent proteins (e.g., EBFP, EBFP2, Azurite, mKalamal, GFPuv, Sapphire, T-sapphire,), cyan fluorescent proteins (e.g., ECFP, Cerulean, CyPet, AmCyan1, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In other embodiments, the marker domain may be a purification tag and/or an epitope tag. Non-limiting exemplary tags include glutathione-S-transferase (GST), chitin binding protein (CBP), maltose binding protein (MBP), thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, S1, T7, V5, VSV-G, 6×His, 8×His, biotin carboxyl carrier protein (BCCP), poly-His, and calmodulin. Non-limiting exemplary reporter genes include glutathione-S-transferase (GST), horseradish peroxidase (HRP), chloramphenicol acetyltransferase (CAT), beta-galactosidase, beta-glucuronidase, luciferase, or fluorescent proteins.

In additional embodiments, the heterologous functional domain may target the RNA-guided DNA-binding agent to a specific organelle, cell type, tissue, or organ. In some embodiments, the heterologous functional domain may target the RNA-guided DNA-binding agent to mitochondria.

In further embodiments, the heterologous functional domain may be an effector domain. When the RNA-guided DNA-binding agent is directed to its target sequence, e.g., when a Cas nuclease is directed to a target sequence by a gRNA, the effector domain may modify or affect the target sequence. In some embodiments, the effector domain may be chosen from a nucleic acid binding domain, a nuclease domain (e.g., a non-Cas nuclease domain), an epigenetic modification domain, a transcriptional activation domain, or a transcriptional repressor domain. In some embodiments, the heterologous functional domain is a nuclease, such as a FokI nuclease. See, e.g., U.S. Pat. No. 9,023,649. In some embodiments, the heterologous functional domain is a transcriptional activator or repressor. See, e.g., Qi et al., “Repurposing CRISPR as an RNA-guided platform for sequence-specific control of gene expression,” Cell 152:1173-83 (2013); Perez-Pinera et al., “RNA-guided gene activation by CRISPR-Cas9-based transcription factors,” Nat. Methods 10:973-6 (2013); Mali et al., “CAS9 transcriptional activators for target specificity screening and paired nickases for cooperative genome engineering,” Nat. Biotechnol. 31:833-8 (2013); Gilbert et al., “CRISPR-mediated modular RNA-guided regulation of transcription in eukaryotes,” Cell 154:442-51 (2013). As such, the RNA-guided DNA-binding agent essentially becomes a transcription factor that can be directed to bind a desired target sequence using a guide RNA. In certain embodiments, the DNA modification domain is a methylation domain, such as a demethylation or methyltransferase domain. In certain embodiments, the effector domain is a DNA modification domain, such as a base-editing domain. In particular embodiments, the DNA modification domain is a nucleic acid editing domain that introduces a specific modification into the DNA, such as a deaminase domain. See, e.g., WO 2015/089406; US 2016/0304846. The nucleic acid editing domains, deaminase domains, and Cas9 variants described in WO 2015/089406 and US 2016/0304846 are hereby incorporated by reference. An RNA-guided DNA binding agent comprising any such domain may be encoded by an ORF disclosed herein, e.g., having an amount of codon pairs of Table 1 described herein optionally in combination with other features described herein.

### 7. UTRs; Kozak Sequences

In some embodiments, the polynucleotide comprises at least one UTR from Hydroxysteroid 17-Beta Dehydrogenase 4 (HSD17B4 or HSD), e.g., a 5′ UTR from HSD. In some embodiments, the polynucleotide comprises at least one UTR from a globin mRNA, for example, human alpha globin (HBA) mRNA, human beta globin (HBB) mRNA, or Xenopus laevis beta globin (XBG) mRNA. In some embodiments, the polynucleotide comprises a 5′ UTR, 3′ UTR, or 5′ and 3′ UTRs from a globin mRNA, such as HBA, HBB, or XBG. In some embodiments, the polynucleotide comprises a 5′ UTR from bovine growth hormone, cytomegalovirus (CMV), mouse Hba-a1, HSD, an albumin gene, HBA, HBB, or XBG. In some embodiments, the polynucleotide comprises a 3′ UTR from bovine growth hormone, cytomegalovirus, mouse Hba-a1, HSD, an albumin gene, HBA, HBB, or XBG. In some embodiments, the polynucleotide comprises 5′ and 3′ UTRs from bovine growth hormone, cytomegalovirus, mouse Hba-a1, HSD, an albumin gene, HBA, HBB, XBG, heat shock protein 90 (Hsp90), glyceraldehyde 3-phosphate dehydrogenase (GAPDH), beta-actin, alpha-tubulin, tumor protein (p53), or epidermal growth factor receptor (EGFR).

In some embodiments, the polynucleotide comprises 5′ and 3′ UTRs that are from the same source, e.g., a constitutively expressed mRNA such as actin, albumin, or a globin such as HBA, HBB, or XBG.

In some embodiments, an mRNA disclosed herein comprises a 5′ UTR with at least 90% identity to any one of SEQ ID NOs: 177-181 or 190-192. In some embodiments, an mRNA disclosed herein comprises a 3′ UTR with at least 90% identity to any one of SEQ ID NOs: 182-186 or 202-204. In some embodiments, any of the foregoing levels of identity is at least 95%, at least 98%, at least 99%, or 100%. In some embodiments, an mRNA disclosed herein comprises a 5′ UTR having the sequence of any one of SEQ ID NOs: 177-181 or 190-192. In some embodiments, an mRNA disclosed herein comprises a 3′ UTR having the sequence of any one of SEQ ID NOs: 182-186 or 202-204.

In some embodiments, the mRNA does not comprise a 5′ UTR, e.g., there are no additional nucleotides between the 5′ cap and the start codon. In some embodiments, the mRNA comprises a Kozak sequence (described below) between the 5′ cap and the start codon, but does not have any additional 5′ UTR. In some embodiments, the mRNA does not comprise a 3′ UTR, e.g., there are no additional nucleotides between the stop codon and the poly-A tail.

In some embodiments, the mRNA comprises a Kozak sequence. The Kozak sequence can affect translation initiation and the overall yield of a polypeptide translated from an mRNA. A Kozak sequence includes a methionine codon that can function as the start codon. A minimal Kozak sequence is NNNRUGN wherein at least one of the following is true: the first N is A or G and the second N is G. In the context of a nucleotide sequence, R means a purine (A or G). In some embodiments, the Kozak sequence is RNNRUGN, NNNRUGG, RNNRUGG, RNNAUGN, NNNAUGG, or RNNAUGG. In some embodiments, the Kozak sequence is rccRUGg with zero mismatches or with up to one or two mismatches to positions in lowercase. In some embodiments, the Kozak sequence is rccAUGg with zero mismatches or with up to one or two mismatches to positions in lowercase. In some embodiments, the Kozak sequence is gccRccAUGG (nucleotides 4-13 of SEQ ID NO: 187) with zero mismatches or with up to one, two, or three mismatches to positions in lowercase. In some embodiments, the Kozak sequence is gccAccAUG with zero mismatches or with up to one, two, three, or four mismatches to positions in lowercase. In some embodiments, the Kozak sequence is GCCACCAUG. In some embodiments, the Kozak sequence is gccgccRccAUGG (SEQ ID NO: 187) with zero mismatches or with up to one, two, three, or four mismatches to positions in lowercase.

### 8. Poly-A Tail

In some embodiments, the polynucleotide is a mRNA that encodes a polypeptide of interest comprising an ORF, and the mRNA further comprises a poly-adenylated (poly-A) tail. In some instances, the poly-A tail is “interrupted” with one or more non-adenine nucleotide “anchors” at one or more locations within the poly-A tail. The poly-A tails may comprise at least 8 consecutive adenine nucleotides, but also comprise one or more non-adenine nucleotide. As used herein, “non-adenine nucleotides” refer to any natural or non-natural nucleotides that do not comprise adenine. Guanine, thymine, and cytosine nucleotides are exemplary non-adenine nucleotides. Thus, the poly-A tails on the mRNA described herein may comprise consecutive adenine nucleotides located 3′ to nucleotides encoding a polypeptide of interest. In some instances, the poly-A tails on mRNA comprise non-consecutive adenine nucleotides located 3′ to nucleotides encoding an RNA-guided DNA-binding agent or a sequence of interest, wherein non-adenine nucleotides interrupt the adenine nucleotides at regular or irregularly spaced intervals.

In some embodiments, the poly-A tail is encoded in the plasmid used for in vitro transcription of mRNA and becomes part of the transcript. The poly-A sequence encoded in the plasmid, i.e., the number of consecutive adenine nucleotides in the poly-A sequence, may not be exact, e.g., a 100 poly-A sequence in the plasmid may not result in a precisely 100 poly-A sequence in the transcribed mRNA. In some embodiments, the poly-A tail is not encoded in the plasmid, and is added by PCR tailing or enzymatic tailing, e.g., using E. coli poly(A) polymerase.

In some embodiments, the one or more non-adenine nucleotides are positioned to interrupt the consecutive adenine nucleotides so that a poly(A) binding protein can bind to a stretch of consecutive adenine nucleotides. In some embodiments, one or more non-adenine nucleotide(s) is located after at least 8, 9, 10, 11, or 12 consecutive adenine nucleotides. In some embodiments, the one or more non-adenine nucleotide is located after at least 8-50 consecutive adenine nucleotides. In some embodiments, the one or more non-adenine nucleotide is located after at least 8-100 consecutive adenine nucleotides. In some embodiments, the non-adenine nucleotide is after one, two, three, four, five, six, or seven adenine nucleotides and is followed by at least 8 consecutive adenine nucleotides.

The poly-A tail of the present disclosure may comprise one sequence of consecutive adenine nucleotides followed by one or more non-adenine nucleotides, optionally followed by additional adenine nucleotides.

In some embodiments, the poly-A tail comprises or contains one non-adenine nucleotide or one consecutive stretch of 2-10 non-adenine nucleotides. In some embodiments, the non-adenine nucleotide(s) is located after at least 8, 9, 10, 11, or 12 consecutive adenine nucleotides. In some instances, the one or more non-adenine nucleotides are located after at least 8-50 consecutive adenine nucleotides. In some embodiments, the one or more non-adenine nucleotides are located after at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 consecutive adenine nucleotides.

In some embodiments, the non-adenine nucleotide is guanine, cytosine, or thymine. In some instances, the non-adenine nucleotide is a guanine nucleotide. In some embodiments, the non-adenine nucleotide is a cytosine nucleotide. In some embodiments, the non-adenine nucleotide is a thymine nucleotide. In some instances, where more than one non-adenine nucleotide is present, the non-adenine nucleotide may be selected from: a) guanine and thymine nucleotides; b) guanine and cytosine nucleotides; c) thymine and cytosine nucleotides; or d) guanine, thymine and cytosine nucleotides. An exemplary poly-A tail comprising non-adenine nucleotides is provided as SEQ ID NO: 188.

### 9. Modified Nucleotides

In some embodiments, a nucleic acid comprising an ORF encoding a polypeptide of interest comprises a modified uridine at some or all uridine positions. In some embodiments, the modified uridine is a uridine modified at the 5 position, e.g., with a halogen or C1-C3 alkoxy. In some embodiments, the modified uridine is a pseudouridine modified at the 1 position, e.g., with a C1-C3 alkyl. The modified uridine can be, for example, pseudouridine, N1-methyl-pseudouridine, 5-methoxyuridine, 5-iodouridine, or a combination thereof. In some embodiments the modified uridine is 5-methoxyuridine. In some embodiments the modified uridine is 5-iodouridine. In some embodiments the modified uridine is pseudouridine. In some embodiments, the modified uridine is N1-methyl-pseudouridine. In some embodiments, the modified uridine is a combination of pseudouridine and N1-methyl-pseudouridine. In some embodiments, the modified uridine is a combination of pseudouridine and 5-methoxyuridine. In some embodiments, the modified uridine is a combination of N1-methyl pseudouridine and 5-methoxyuridine. In some embodiments, the modified uridine is a combination of 5-iodouridine and N1-methyl-pseudouridine. In some embodiments, the modified uridine is a combination of pseudouridine and 5-iodouridine. In some embodiments, the modified uridine is a combination of 5-iodouridine and 5-methoxyuridine.

In some embodiments, at least 10%, 15%, 20%, 25%, 30%, 35%, 40%, 45%, 50%, 55%, 60%, 65%, 70%, 75%, 80%, 85%, 90%, 95%, 98%, 99%, or 100% of the uridine positions in a polynucleotide according to the disclosure are modified uridines. In some embodiments, 10%-25%, 15-25%, 25-35%, 35-45%, 45-55%, 55-65%, 65-75%, 75-85%, 85-95%, or 90-100% of the uridine positions in a polynucleotide according to the disclosure are modified uridines, e.g., 5-methoxyuridine, 5-iodouridine, N1-methyl pseudouridine, pseudouridine, or a combination thereof. In some embodiments, 10%-25%, 15-25%, 25-35%, 35-45%, 45-55%, 55-65%, 65-75%, 75-85%, 85-95%, or 90-100% of the uridine positions in a polynucleotide according to the disclosure are 5-methoxyuridine. In some embodiments, 10%-25%, 15-25%, 25-35%, 35-45%, 45-55%, 55-65%, 65-75%, 75-85%, 85-95%, or 90-100% of the uridine positions in a polynucleotide according to the disclosure are pseudouridine. In some embodiments, 10%-25%, 15-25%, 25-35%, 35-45%, 45-55%, 55-65%, 65-75%, 75-85%, 85-95%, or 90-100% of the uridine positions in a polynucleotide according to the disclosure are N1-methyl pseudouridine. In some embodiments, 10%-25%, 15-25%, 25-35%, 35-45%, 45-55%, 55-65%, 65-75%, 75-85%, 85-95%, or 90-100% of the uridine positions in a polynucleotide according to the disclosure are 5-iodouridine. In some embodiments, 10%-25%, 15-25%, 25-35%, 35-45%, 45-55%, 55-65%, 65-75%, 75-85%, 85-95%, or 90-100% of the uridine positions in a polynucleotide according to the disclosure are 5-methoxyuridine, and the remainder are N1-methyl pseudouridine. In some embodiments, 10%-25%, 15-25%, 25-35%, 35-45%, 45-55%, 55-65%, 65-75%, 75-85%, 85-95%, or 90-100% of the uridine positions in a polynucleotide according to the disclosure are 5-iodouridine, and the remainder are N1-methyl pseudouridine. In some embodiments, 15% to 45%, 45% to 55%, 55% to 65%, 65% to 75%, 75% to 85%, 85% to 95%, or 90% to 100% of the uridine positions in a polynucleotide according to the disclosure is substituted with the modified uridine, optionally wherein the modified uridine is N1-methyl-pseudouridine. In some embodiments, 15% to 45%, 45% to 55%, 55% to 65%, 65% to 75%, 75% to 85%, 85% to 95%, or 90% to 100% of the uridine positions in a polynucleotide according to the disclosure is substituted with N1-methyl-pseudouridine. In some embodiments, 85%, 90%, 95%, or 100% of the uridine positions in a polynucleotide according to the disclosure is substituted with N1-methyl-pseudouridine. In some embodiments, 100% of the uridine is substituted with N1-methyl-pseudouridine. In some embodiments, 15% to 45%, 45% to 55%, 55% to 65%, 65% to 75%, 75% to 85%, 85% to 95%, or 90% to 100% of the uridine positions in a polynucleotide according to the disclosure is substituted with the modified uridine, optionally wherein the modified uridine is pseudouridine. In some embodiments, 15% to 45%, 45% to 55%, 55% to 65%, 65% to 75%, 75% to 85%, 85% to 95%, or 90% to 100% of the uridine positions in a polynucleotide according to the disclosure is substituted with pseudouridine. In some embodiments, 85%, 90%, 95%, or 100% of the uridine positions in a polynucleotide according to the disclosure is substituted with pseudouridine. In some embodiments, 100% of the uridine is substituted with pseudouridine.

10.5′ Cap

In some embodiments, a nucleic acid (e.g., mRNA) disclosed herein comprises a 5′ cap, such as a Cap0, Cap1, or Cap2. A 5′ cap is generally a 7-methylguanine ribonucleotide (which may be further modified, as discussed below e.g. with respect to ARCA) linked through a 5′-triphosphate to the 5′ position of the first nucleotide of the 5′-to-3′ chain of the nucleic acid, i.e., the first cap-proximal nucleotide. In Cap0, the riboses of the first and second cap-proximal nucleotides of the mRNA both comprise a 2′-hydroxyl. In Cap1, the riboses of the first and second transcribed nucleotides of the mRNA comprise a 2′-methoxy and a 2′-hydroxyl, respectively. In Cap2, the riboses of the first and second cap-proximal nucleotides of the mRNA both comprise a 2′-methoxy. See, e.g., Katibah et al. (2014) Proc Natl Acad Sci USA 111(33):12025-30; Abbas et al. (2017) Proc Natl Acad Sci USA 114(11):E2106-E2115. Most endogenous higher eukaryotic nucleic acids, including mammalian nucleic acids such as human nucleic acids, comprise Cap1 or Cap2. Cap0 and other cap structures differing from Cap1 and Cap2 may be immunogenic in mammals, such as humans, due to recognition as “non-self” by components of the innate immune system such as IFIT-1 and IFIT-5, which can result in elevated cytokine levels including type I interferon. Components of the innate immune system such as IFIT-1 and IFIT-5 may also compete with eIF4E for binding of a nucleic acids with a cap other than Cap1 or Cap2, potentially inhibiting translation of the nucleic acid.

A cap can be included co-transcriptionally. For example, ARCA (anti-reverse cap analog; Thermo Fisher Scientific Cat. No. AM8045) is a cap analog comprising a 7-methylguanine 3′-methoxy-5′-triphosphate linked to the 5′ position of a guanine ribonucleotide which can be incorporated in vitro into a transcript at initiation. ARCA results in a Cap0 cap or a Cap0-like cap in which the 2′ position of the first cap-proximal nucleotide is hydroxyl. See, e.g., Stepinski et al., (2001) “Synthesis and properties of mRNAs containing the novel ‘anti-reverse’ cap analogs 7-methyl(3′-O-methyl)GpppG and 7-methyl(3′deoxy)GpppG,” RNA 7: 1486-1495. The ARCA structure is shown below.

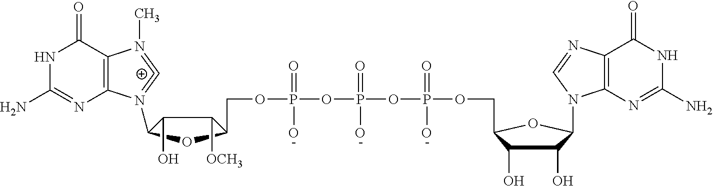

CleanCap™ AG (m7G(5′)ppp(5′)(2′OMeA)pG; TriLink Biotechnologies Cat. No. N-7113) or CleanCap™ GG (m7G(5′)ppp(5′)(2′OMeG)pG; TriLink Biotechnologies Cat. No. N-7133) can be used to provide a Cap1 structure co-transcriptionally. 3′-O-methylated versions of CleanCap™ AG and CleanCap™ GG are also available from TriLink Biotechnologies as Cat. Nos. N-7413 and N-7433, respectively. The CleanCap™ AG structure is shown below. CleanCap™ structures are sometimes referred to herein using the last three digits of the catalog numbers listed above (e.g., “CleanCap™ 113” for TriLink Biotechnologies Cat. No. N-7113).

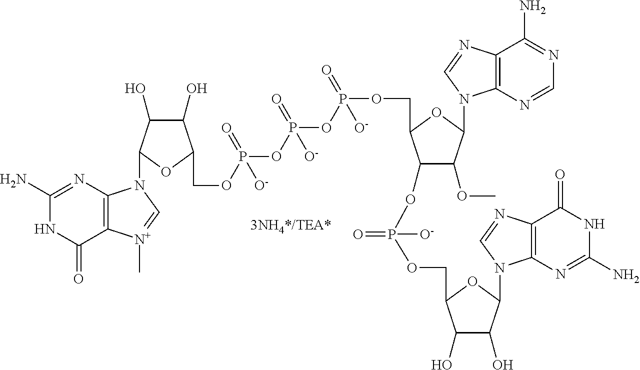 [SMILES: COC1C(OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(O)C2O)OC1n1cnc2c(N)ncnc21]

Alternatively, a cap can be added to an RNA post-transcriptionally. For example, Vaccinia capping enzyme is commercially available (New England Biolabs Cat. No. M2080S) and has RNA triphosphatase and guanylyltransferase activities, provided by its D1 subunit, and guanine methyltransferase, provided by its D12 subunit. As such, it can add a 7-methylguanine to an RNA, so as to give Cap0, in the presence of S-adenosyl methionine and GTP. See, e.g., Guo, P. and Moss, B. (1990) Proc. Natl. Acad. Sci. USA 87, 4023-4027; Mao, X. and Shuman, S. (1994) J. Biol. Chem. 269, 24472-24479. For additional discussion of caps and capping approaches, see, e.g., WO2017/053297 and Ishikawa et al., Nucl. Acids. Symp. Ser. (2009) No. 53, 129-130.

### 11. Guide RNA

In some embodiments, at least one guide RNA is provided in combination with a polynucleotide disclosed herein, such as a polynucleotide encoding an RNA-guided DNA-binding agent. In some embodiments, a guide RNA is provided as a separate molecule from the polynucleotide. In some embodiments, a guide RNA is provided as a part, such as a part of a UTR, of a polynucleotide disclosed herein. In some embodiments, at least one guide RNA targets TTR.

In some embodiments, a guide RNA comprises a modified sgRNA. An sgRNA may be modified to improve its in vivo stability. In some embodiments, the sgRNA comprises the modification pattern shown in SEQ ID NO: 189, where N is any natural or non-natural nucleotide, and where the totality of the N's comprises a guide sequence. The modifications are as shown in SEQ ID NO: 189 despite the substitution of N's for the nucleotides of a guide. That is, although the nucleotides of the guide replace the “N's”, the first three nucleotides are 2′OMe modified and there are phosphorothioate linkages between the first and second nucleotides, the second and third nucleotides and the third and fourth nucleotides.

### 12. Lipids; Formulation; Delivery

In some embodiments, a polynucleotide described herein is formulated in or administered via a lipid nanoparticle; see, e.g., WO2017173054A1 published Oct. 5, 2017, the contents of which are hereby incorporated by reference in their entirety. Any lipid nanoparticle (LNP) known to those of skill in the art to be capable of delivering nucleotides to subjects may be utilized to administer the polynucleotides described herein, in some embodiments, optionally accompanied by other nucleic acid component(s) such as guide RNAs. In some embodiments, a polynucleotide described herein, alone or optionally accompanied by other nucleic acid component(s), is formulated in or administered via liposome, a nanoparticle, an exosome, or a microvesicle. Emulsions, micelles, and suspensions may be suitable compositions for local and/or topical delivery.

Disclosed herein are various embodiments of LNP formulations for nucleic acids. Such LNP formulations may include a biodegradable ionizable lipid. Formulations may include, e.g. (i) a CCD lipid, such as an amine lipid, optionally including one or more of (ii) a neutral lipid, (iii) a helper lipid, and (iv) a stealth lipid, such as a PEG lipid. Some embodiments of the LNP formulations include an “amine lipid”, along with a helper lipid, a neutral lipid, and a stealth lipid such as a PEG lipid. By “lipid nanoparticle” is meant a particle that comprises a plurality of (i.e. more than one) lipid molecules physically associated with each other by intermolecular forces.

CCD Lipids

Lipid compositions for delivery of polynucleotide components to a liver cell may comprise a CCD Lipid, or for example, another biodegradable lipid.

In some embodiments, the CCD lipid is Lipid A, which is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. Lipid A can be depicted as:

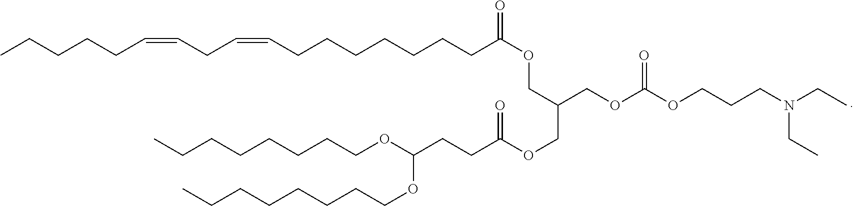 [SMILES: CCCCC/C=C\C/C=C\CCCCCCCC(=O)OCC(COC(=O)CCC(OCCCCCCCC)OCCCCCCCC)COC(=O)OCCCN(CC)CC]

Lipid A may be synthesized according to WO2015/095340 (e.g., pp. 84-86).

In some embodiments, the CCD lipid is Lipid B, which is ((5-((dimethylamino)methyl)-1,3-phenylene)bis(oxy))bis(octane-8,1-diyl)bis(decanoate), also called ((5-((dimethylamino)methyl)-1,3-phenylene)bis(oxy))bis(octane-8,1-diyl) bis(decanoate). Lipid B can be depicted as:

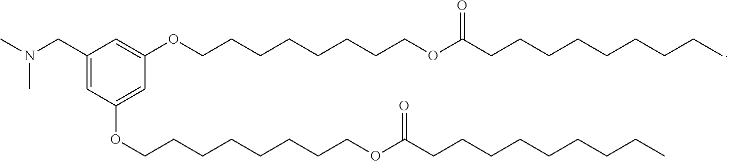 [SMILES: CCCCCCCCCC(=O)OCCCCCCCCOc1cc(CN(C)C)cc(OCCCCCCCCOC(=O)CCCCCCCCC)c1]

Lipid B may be synthesized according to WO2014/136086 (e.g., pp. 107-09).
In some embodiments, the CCD lipid is Lipid C, which is 2-((4-(((3-(dimethylamino)propoxy)carbonyl)oxy)hexadecanoyl)oxy)propane-1,3-diyl (9Z,9′Z,12Z,12′Z)-bis(octadeca-9,12-dienoate).
Lipid C can be depicted as:

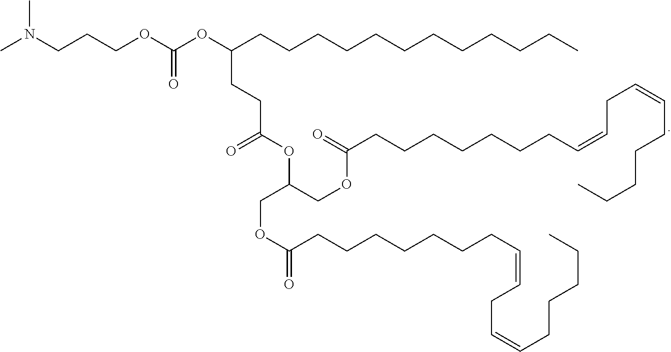 [SMILES: CCCCC/C=C\C/C=C\CCCCCCCC(=O)OCC(COC(=O)CCCCCCC/C=C\C/C=C\CCCCC)OC(=O)CCC(CCCCCCCCCCCC)OC(=O)OCCCN(C)C]

In some embodiments, the CCD lipid is Lipid D, which is 3-(((3-(dimethylamino)propoxy)carbonyl)oxy)-13-(octanoyloxy)tridecyl 3-octylundecanoate.

Lipid D can be depicted as:

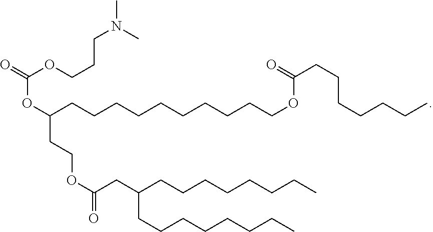 [SMILES: CCCCCCCCC(CCCCCCCC)CC(=O)OCCC(CCCCCCCCCCOC(=O)CCCCCCC)OC(=O)OCCCN(C)C]

Lipid C and Lipid D may be synthesized according to WO2015/095340.

The CCD lipid can also be an equivalent to Lipid A, Lipid B, Lipid C, or Lipid D. In certain embodiments, the CCD lipid is an equivalent to Lipid A, an equivalent to Lipid B, an equivalent to Lipid C, or an equivalent to Lipid D.

Amine Lipids

In some embodiments, the LNP compositions for the delivery of biologically active agents comprise an “amine lipid”, which is defined as Lipid A or its equivalents, including acetal analogs of Lipid A.

In some embodiments, the amine lipid is Lipid A, which is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. Lipid A can be depicted as:

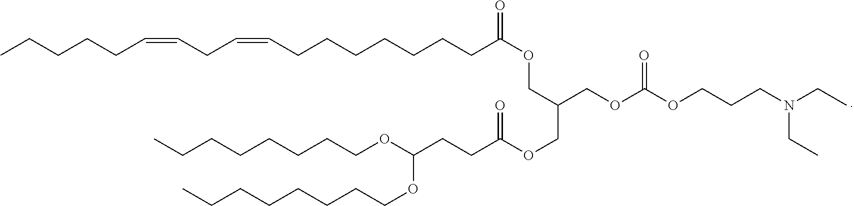 [SMILES: CCCCC/C=C\C/C=C\CCCCCCCC(=O)OCC(COC(=O)CCC(OCCCCCCCC)OCCCCCCCC)COC(=O)OCCCN(CC)CC]

Lipid A may be synthesized according to WO2015/095340 (e.g., pp. 84-86). In certain embodiments, the amine lipid is an equivalent to Lipid A.

In certain embodiments, an amine lipid is an analog of Lipid A. In certain embodiments, a Lipid A analog is an acetal analog of Lipid A. In particular LNP compositions, the acetal analog is a C4-C12 acetal analog. In some embodiments, the acetal analog is a C5-C12 acetal analog. In additional embodiments, the acetal analog is a C5-C10 acetal analog. In further embodiments, the acetal analog is chosen from a C4, C5, C6, C7, C9, C10, C11, and C12 acetal analog.

Amine lipids suitable for use in the LNPs described herein are biodegradable in vivo. The amine lipids have low toxicity (e.g., are tolerated in animal models without adverse effect in amounts of greater than or equal to 10 mg/kg). In certain embodiments, LNPs comprising an amine lipid include those where at least 75% of the amine lipid is cleared from the plasma within 8, 10, 12, 24, or 48 hours, or 3, 4, 5, 6, 7, or 10 days. In certain embodiments, LNPs comprising an amine lipid include those where at least 50% of the polynucleotide or other component is cleared from the plasma within 8, 10, 12, 24, or 48 hours, or 3, 4, 5, 6, 7, or 10 days. In certain embodiments, LNPs comprising an amine lipid include those where at least 50% of the LNP is cleared from the plasma within 8, 10, 12, 24, or 48 hours, or 3, 4, 5, 6, 7, or 10 days, for example by measuring a lipid (e.g., an amine lipid), polynucleotide (e.g., mRNA), or other component. In certain embodiments, lipid-encapsulated versus free lipid, polynucleotide, or other nucleic acid component of the LNP is measured.

Lipid clearance may be measured as described in literature. See Maier, M. A., et al. Biodegradable Lipids Enabling Rapidly Eliminated Lipid Nanoparticles for Systemic Delivery of RNAi Therapeutics. Mol. Ther. 2013, 21(8), 1570-78 (“Maier”). For example, in Maier, LNP-siRNA systems containing luciferases-targeting siRNA were administered to six- to eight-week old male C57Bl/6 mice at 0.3 mg/kg by intravenous bolus injection via the lateral tail vein. Blood, liver, and spleen samples were collected at 0.083, 0.25, 0.5, 1, 2, 4, 8, 24, 48, 96, and 168 hours post-dose. Mice were perfused with saline before tissue collection and blood samples were processed to obtain plasma. All samples were processed and analyzed by LC-MS. Further, Maier describes a procedure for assessing toxicity after administration of LNP-siRNA formulations. For example, a luciferase-targeting siRNA was administered at 0, 1, 3, 5, and 10 mg/kg (5 animals/group) via single intravenous bolus injection at a dose volume of 5 mL/kg to male Sprague-Dawley rats. After 24 hours, about 1 mL of blood was obtained from the jugular vein of conscious animals and the serum was isolated. At 72 hours post-dose, all animals were euthanized for necropsy. Assessment of clinical signs, body weight, serum chemistry, organ weights and histopathology was performed. Although Maier describes methods for assessing siRNA-LNP formulations, these methods may be applied to assess clearance, pharmacokinetics, and toxicity of administration of LNP compositions of the present disclosure.

The amine lipids lead to an increased clearance rate. In some embodiments, the clearance rate is a lipid clearance rate, for example the rate at which an amine lipid is cleared from the blood, serum, or plasma. In some embodiments, the clearance rate is a polynucleotide clearance rate, for example the rate at a polynucleotide is cleared from the blood, serum, or plasma. In some embodiments, the clearance rate is the rate at which LNP is cleared from the blood, serum, or plasma. In some embodiments, the clearance rate is the rate at which LNP is cleared from a tissue, such as liver tissue or spleen tissue. In certain embodiments, a high rate of clearance rate leads to a safety profile with no substantial adverse effects. The amine lipids reduce LNP accumulation in circulation and in tissues. In some embodiments, a reduction in LNP accumulation in circulation and in tissues leads to a safety profile with no substantial adverse effects.

The amine lipids of the present disclosure may be ionizable depending upon the pH of the medium they are in. For example, in a slightly acidic medium, the amine lipids may be protonated and thus bear a positive charge. Conversely, in a slightly basic medium, such as, for example, blood where pH is approximately 7.35, the amine lipids may not be protonated and thus bear no charge. In some embodiments, the amine lipids of the present disclosure may be protonated at a pH of at least about 9. In some embodiments, the amine lipids of the present disclosure may be protonated at a pH of at least about 9. In some embodiments, the amine lipids of the present disclosure may be protonated at a pH of at least about 10.

The ability of an amine lipid to bear a charge is related to its intrinsic pKa. For example, the amine lipids of the present disclosure may each, independently, have a pKa in the range of from about 5.8 to about 6.2. For example, the amine lipids of the present disclosure may each, independently, have a pKa in the range of from about 5.8 to about 6.5. This may be advantageous as it has been found that cationic lipids with a pKa ranging from about 5.1 to about 7.4 are effective for delivery of cargo in vivo, e.g. to the liver. Further, it has been found that cationic lipids with a pKa ranging from about 5.3 to about 6.4 are effective for delivery in vivo, e.g. to tumors. See, e.g., WO2014/136086.

Additional Lipids

“Neutral lipids” suitable for use in a lipid composition of the disclosure include, for example, a variety of neutral, uncharged or zwitterionic lipids. Examples of neutral phospholipids suitable for use in the present disclosure include, but are not limited to, 5-heptadecylbenzene-1,3-diol (resorcinol), dipalmitoylphosphatidylcholine (DPPC), distearoylphosphatidylcholine (DSPC), dioleoylphosphatidylcholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine and combinations thereof. In one embodiment, the neutral phospholipid may be selected from the group consisting of distearoylphosphatidylcholine (DSPC) and dimyristoyl phosphatidyl ethanolamine (DMPE). In another embodiment, the neutral phospholipid may be distearoylphosphatidylcholine (DSPC).

“Helper lipids” include steroids, sterols, and alkyl resorcinols. Helper lipids suitable for use in the present disclosure include, but are not limited to, cholesterol, 5-heptadecylresorcinol, and cholesterol hemisuccinate. In one embodiment, the helper lipid may be cholesterol. In one embodiment, the helper lipid may be cholesterol hemisuccinate.

“Stealth lipids” are lipids that alter the length of time the nanoparticles can exist in vivo (e.g., in the blood). Stealth lipids may assist in the formulation process by, for example, reducing particle aggregation and controlling particle size. Stealth lipids used herein may modulate pharmacokinetic properties of the LNP. Stealth lipids suitable for use in a lipid composition of the disclosure include, but are not limited to, stealth lipids having a hydrophilic head group linked to a lipid moiety. Stealth lipids suitable for use in a lipid composition of the present disclosure and information about the biochemistry of such lipids can be found in Romberg et al., Pharmaceutical Research, Vol. 25, No. 1, 2008, pg. 55-71 and Hoekstra et al., Biochimica et Biophysica Acta 1660 (2004) 41-52. Additional suitable PEG lipids are disclosed, e.g., in WO 2006/007712.

In one embodiment, the hydrophilic head group of stealth lipid comprises a polymer moiety selected from polymers based on PEG. Stealth lipids may comprise a lipid moiety. In some embodiments, the stealth lipid is a PEG lipid.

In one embodiment, a stealth lipid comprises a polymer moiety selected from polymers based on PEG (sometimes referred to as poly(ethylene oxide)), poly(oxazoline), poly(vinyl alcohol), poly(glycerol), poly(N-vinylpyrrolidone), polyaminoacids and poly[N-(2-hydroxypropyl)methacrylamide].

In one embodiment, the PEG lipid comprises a polymer moiety based on PEG (sometimes referred to as poly(ethylene oxide)).

The PEG lipid further comprises a lipid moiety. In some embodiments, the lipid moiety may be derived from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. In some embodiments, the alkyl chail length comprises about C10 to C20. The dialkylglycerol or dialkylglycamide group can further comprise one or more substituted alkyl groups. The chain lengths may be symmetrical or assymetric.

Unless otherwise indicated, the term “PEG” as used herein means any polyethylene glycol or other polyalkylene ether polymer. In one embodiment, PEG is an optionally substituted linear or branched polymer of ethylene glycol or ethylene oxide. In one embodiment, PEG is unsubstituted. In one embodiment, the PEG is substituted, e.g., by one or more alkyl, alkoxy, acyl, hydroxy, or aryl groups. In one embodiment, the term includes PEG copolymers such as PEG-polyurethane or PEG-polypropylene (see, e.g., J. Milton Harris, Poly(ethylene glycol) chemistry: biotechnical and biomedical applications (1992)); in another embodiment, the term does not include PEG copolymers. In one embodiment, the PEG has a molecular weight of from about 130 to about 50,000, in a sub-embodiment, about 150 to about 30,000, in a sub-embodiment, about 150 to about 20,000, in a sub-embodiment about 150 to about 15,000, in a sub-embodiment, about 150 to about 10,000, in a sub-embodiment, about 150 to about 6,000, in a sub-embodiment, about 150 to about 5,000, in a sub-embodiment, about 150 to about 4,000, in a sub-embodiment, about 150 to about 3,000, in a sub-embodiment, about 300 to about 3,000, in a sub-embodiment, about 1,000 to about 3,000, and in a sub-embodiment, about 1,500 to about 2,500.

In certain embodiments, the PEG (e.g., conjugated to a lipid moiety or lipid, such as a stealth lipid), is a “PEG-2K,” also termed “PEG 2000,” which has an average molecular weight of about 2,000 daltons. PEG-2K is represented herein by the following formula (I), wherein n is 45, meaning that the number averaged degree of polymerization comprises about 45 subunits. However, other PEG embodiments known in the art may be used, including, e.g., those where the number-averaged degree of polymerization comprises about 23 subunits (n=23), and/or 68 subunits (n=68). In some embodiments, n may range from about 30 to about 60. In some embodiments, n may range from about 35 to about 55. In some embodiments, n may range from about 40 to about 50. In some embodiments, n may range from about 42 to about 48. In some embodiments, n may be 45. In some embodiments, R may be selected from H, substituted alkyl, and unsubstituted alkyl. In some embodiments, R may be unsubstituted alkyl. In some embodiments, R may be methyl.

In any of the embodiments described herein, the PEG lipid may be selected from PEG-dilauroylglycerol, PEG-dimyristoylglycerol (PEG-DMG) (catalog #GM-020 from NOF, Tokyo, Japan), PEG-dipalmitoylglycerol, PEG-distearoylglycerol (PEG-DSPE) (catalog #DSPE-020CN, NOF, Tokyo, Japan), PEG-dilaurylglycamide, PEG-dimyristylglycamide, PEG-dipalmitoylglycamide, and PEG-distearoylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-ditetradecoxylbenzy-[omega]-methyl-poly(ethylene glycol)ether), 1,2-dimyristoyl-sn-glycero-3-phosphoethanolamine-N-[methoxy(polyethylene glycol)-2000] (PEG2k-DMG), 1,2-distearoyl-sn-glycero-3-phosphoethanolamine-N-[methoxy(polyethylene glycol)-2000] (PEG2k-DSPE) (cat. #880120C from Avanti Polar Lipids, Alabaster, Ala., USA), 1,2-distearoyl-sn-glycerol, methoxypolyethylene glycol (PEG2k-DSG; GS-020, NOF Tokyo, Japan), poly(ethylene glycol)-2000-dimethacrylate (PEG2k-DMA), and 1,2-distearyloxypropyl-3-amine-N-[methoxy(polyethylene glycol)-2000] (PEG2k-DSA). In one embodiment, the PEG lipid may be PEG2k-DMG. In some embodiments, the PEG lipid may be PEG2k-DSG. In one embodiment, the PEG lipid may be PEG2k-DSPE. In one embodiment, the PEG lipid may be PEG2k-DMA. In one embodiment, the PEG lipid may be PEG2k-C-DMA. In one embodiment, the PEG lipid may be compound 5027, disclosed in WO2016/010840 (paragraphs [00240] to [00244]). In one embodiment, the PEG lipid may be PEG2k-DSA. In one embodiment, the PEG lipid may be PEG2k-C11. In some embodiments, the PEG lipid may be PEG2k-C14. In some embodiments, the PEG lipid may be PEG2k-C16. In some embodiments, the PEG lipid may be PEG2k-C18.

LNP Formulations

The LNP may contain an ionizable lipid, for example a biodegradable ionizable lipid suitable for delivery of nucleic acid cargoes. The LNP may contain (i) a CCD or amine lipid for encapsulation and for endosomal escape. Such components may optionally be included in the LNP in combination with one or more of (ii) a neutral lipid for stabilization, (iii) a helper lipid, also for stabilization, and (iv) a stealth lipid, such as a PEG lipid.

In some embodiments, an LNP composition may comprise one or more nucleic acid components that include a polynucleotide comprising an open reading frame (ORF) encoding a polypeptide of interest such as any of those described herein, e.g., an RNA-guided DNA-binding agent. In some embodiments, the nucleic acid component may include a mRNA comprising an open reading frame (ORF) encoding a polypeptide of interest, such as an RNA-guided DNA-binding agent (e.g., a Class 2 Cas nuclease) and optionally a gRNA. In certain embodiments, an LNP composition may comprise the nucleic acid component, an amine lipid, a helper lipid, a neutral lipid, and a stealth lipid. In certain LNP compositions, the helper lipid is cholesterol. In other compositions, the neutral lipid is DSPC. In additional embodiments, the stealth lipid is PEG2k-DMG or PEG2k-C11. In certain embodiments, the LNP composition comprises Lipid A or an equivalent of Lipid A; a helper lipid; a neutral lipid; a stealth lipid; and a nucleic acid component. In certain compositions, the amine lipid is Lipid A. In certain compositions, the amine lipid is Lipid A or an acetal analog thereof; the helper lipid is cholesterol; the neutral lipid is DSPC; and the stealth lipid is PEG2k-DMG.

In certain embodiments, the nucleic acid component includes a polynucleotide comprising an open reading frame (ORF) encoding a polypeptide of interest. In some embodiments, the nucleic acid component includes an RNA-guided DNA-binding agent (e.g. a Cas nuclease, a Class 2 Cas nuclease, or Cas9). In some embodiments, the nucleic acid component includes a gRNA or a nucleic acid encoding a gRNA. In some embodiments, the nucleic acid component includes a combination of mRNA and gRNA. In one embodiment, an LNP composition may comprise a Lipid A or its equivalents. In some aspects, the amine lipid is Lipid A. In some aspects, the amine lipid is a Lipid A equivalent, e.g. an analog of Lipid A. In certain aspects, the amine lipid is an acetal analog of Lipid A. In various embodiments, an LNP composition comprises an amine lipid, a neutral lipid, a helper lipid, and a PEG lipid. In certain embodiments, the helper lipid is cholesterol. In certain embodiments, the neutral lipid is DSPC. In some embodiments, the PEG lipid is PEG2k-DMG. In some embodiments, an LNP composition may comprise a Lipid A, a helper lipid, a neutral lipid, and a PEG lipid. In some embodiments, an LNP composition comprises an amine lipid, DSPC, cholesterol, and a PEG lipid. In some embodiments, the LNP composition comprises a PEG lipid comprising DMG. In certain embodiments, the amine lipid is selected from Lipid A, and an equivalent of Lipid A, including an acetal analog of Lipid A. In additional embodiments, an LNP composition comprises Lipid A, cholesterol, DSPC, and PEG2k-DMG.

Embodiments of the present disclosure also provide lipid compositions described according to the molar ratio between the positively charged amine groups of the amine lipid (N) and the negatively charged phosphate groups (P) of the nucleic acid to be encapsulated. This may be mathematically represented by the equation N/P. In some embodiments, an LNP composition may comprise a lipid component that comprises an amine lipid, a helper lipid, a neutral lipid, and a helper lipid; and a nucleic acid component, wherein the N/P ratio is about 3 to 10. In some embodiments, an LNP composition may comprise a lipid component that comprises an amine lipid, a helper lipid, a neutral lipid, and a helper lipid; and an RNA component, wherein the N/P ratio is about 3 to 10. In one embodiment, the N/P ratio may about 5-7. In one embodiment, the N/P ratio may about 4.5-8. In one embodiment, the N/P ratio may about 6. In one embodiment, the N/P ratio may be 6±1. In one embodiment, the N/P ratio may about 6±0.5. In some embodiments, the N/P ratio will be ±30%, ±25%, ±20%, ±15%, ±10%, ±5%, or ±2.5% of the target N/P ratio. In certain embodiments, LNP inter-lot variability will be less than 15%, less than 10% or less than 5%. In certain embodiments, the LNP compositions include a polynucleotide comprising an open reading frame (ORF) encoding a polypeptide of interest, and additional nucleic acid component such as a gRNA. In certain embodiments, the LNP composition includes a ratio of the polynucleotide component to the other nucleic acid component from about 25:1 to about 1:25. In certain embodiments, the LNP formulation includes a ratio of the polynucleotide component to the other nucleic acid component from about 10:1 to about 1:10. In certain embodiments, the LNP formulation includes a ratio of the polynucleotide component to the other nucleic acid component from about 8:1 to about 1:8. As measured herein, the ratios are by weight. In some embodiments, ratio range is about 5:1 to about 1:5, about 3:1 to 1:3, about 2:1 to 1:2, about 5:1 to 1:2, about 5:1 to 1:1, about 3:1 to 1:2, about 3:1 to 1:1, about 3:1, about 2:1 to 1:1. The ratio may be about 25:1, 10:1, 5:1, 3:1, 1:1, 1:3, 1:5, 1:10, or 1:25.

Optionally, the LNP compositions disclosed herein may include a template nucleic acid. The template nucleic acid may be co-formulated with an mRNA encoding a Cas nuclease, such as a Class 2 Cas nuclease mRNA. In some embodiments, the template nucleic acid may be co-formulated with a guide RNA. In some embodiments, the template nucleic acid may be co-formulated with both an mRNA encoding a Cas nuclease and a guide RNA. In some embodiments, the template nucleic acid may be formulated separately from an mRNA encoding a Cas nuclease or a guide RNA. The template nucleic acid may be delivered with, or separately from the LNP compositions. In some embodiments, the template nucleic acid may be single- or double-stranded, depending on the desired repair mechanism. The template may have regions of homology to the target DNA, or to sequences adjacent to the target DNA.

Any of the LNPs and LNP formulations described herein are suitable for delivery a polynucleotide disclosed herein, alone or together with other nucleic acid components. In some embodiments, an LNP composition is encompassed comprising: a nucleic acid component and a lipid component, wherein the lipid component comprises an amine lipid, a neutral lipid, a helper lipid, and a stealth lipid; and wherein the nucleic acid to lipid (N/P) ratio is about 1-10. In any of the foregoing embodiments, the polynucleotide may be an mRNA.

In some embodiments, LNPs are formed by mixing an aqueous nucleic acid solution with an organic solvent-based lipid solution, e.g., 100% ethanol. Suitable solutions or solvents include or may contain: water, PBS, Tris buffer, NaCl, citrate buffer, ethanol, chloroform, diethylether, cyclohexane, tetrahydrofuran, methanol, isopropanol. A pharmaceutically acceptable buffer, e.g., for in vivo administration of LNPs, may be used. In certain embodiments, a buffer is used to maintain the pH of the composition comprising LNPs at or above pH 6.5. In certain embodiments, a buffer is used to maintain the pH of the composition comprising LNPs at or above pH 7.0. In certain embodiments, the composition has a pH ranging from about 7.2 to about 7.7. In additional embodiments, the composition has a pH ranging from about 7.3 to about 7.7 or ranging from about 7.4 to about 7.6. In further embodiments, the composition has a pH of about 7.2, 7.3, 7.4, 7.5, 7.6, or 7.7. The pH of a composition may be measured with a micro pH probe. In certain embodiments, a cryoprotectant is included in the composition. Non-limiting examples of cryoprotectants include sucrose, trehalose, glycerol, DMSO, and ethylene glycol. Exemplary compositions may include up to 10% cryoprotectant, such as, for example, sucrose. In certain embodiments, the LNP composition may include about 1, 2, 3, 4, 5, 6, 7, 8, 9, or 10% cryoprotectant. In certain embodiments, the LNP composition may include about 1, 2, 3, 4, 5, 6, 7, 8, 9, or 10% sucrose. In some embodiments, the LNP composition may include a buffer. In some embodiments, the buffer may comprise a phosphate buffer (PBS), a Tris buffer, a citrate buffer, and mixtures thereof. In certain exemplary embodiments, the buffer comprises NaCl. In certain embodiments, NaCl is omitted. Exemplary amounts of NaCl may range from about 20 mM to about 45 mM. Exemplary amounts of NaCl may range from about 40 mM to about 50 mM. In some embodiments, the amount of NaCl is about 45 mM. In some embodiments, the buffer is a Tris buffer. Exemplary amounts of Tris may range from about 20 mM to about 60 mM. Exemplary amounts of Tris may range from about 40 mM to about 60 mM. In some embodiments, the amount of Tris is about 50 mM. In some embodiments, the buffer comprises NaCl and Tris. Certain exemplary embodiments of the LNP compositions contain 5% sucrose and 45 mM NaCl in Tris buffer. In other exemplary embodiments, compositions contain sucrose in an amount of about 5% w/v, about 45 mM NaCl, and about 50 mM Tris at pH 7.5. The salt, buffer, and cryoprotectant amounts may be varied such that the osmolality of the overall formulation is maintained. For example, the final osmolality may be maintained at less than 450 mOsm/L. In further embodiments, the osmolality is between 350 and 250 mOsm/L. Certain embodiments have a final osmolality of 300+/−20 mOsm/L.

In some embodiments, microfluidic mixing, T-mixing, or cross-mixing is used. In certain aspects, flow rates, junction size, junction geometry, junction shape, tube diameter, solutions, and/or nucleic acid and lipid concentrations may be varied. LNPs or LNP compositions may be concentrated or purified, e.g., via dialysis, tangential flow filtration, or chromatography. The LNPs may be stored as a suspension, an emulsion, or a lyophilized powder, for example. In some embodiments, an LNP composition is stored at 2-8° C., in certain aspects, the LNP compositions are stored at room temperature. In additional embodiments, an LNP composition is stored frozen, for example at −20° C. or −80° C. In other embodiments, an LNP composition is stored at a temperature ranging from about 0° C. to about −80° C. Frozen LNP compositions may be thawed before use, for example on ice, at 4° C., at room temperature, or at 25° C. Frozen LNP compositions may be maintained at various temperatures, for example on ice, at 4° C., at room temperature, at 25° C., or at 37° C.

In some embodiments, an LNP composition has greater than about 80% encapsulation. In some embodiments, an LNP composition has a particle size less than about 120 nm. In some embodiments, an LNP composition has a pdi less than about 0.2. In some embodiments, at least two of these features are present. In some embodiments, each of these three features is present. Analytical methods for determining these parameters are discussed below in the general reagents and methods section.

In some embodiments, LNPs associated with a polynucleotide disclosed herein are for use in preparing a medicament.

Electroporation is also a well-known means for delivery of nucleic acid components, and any electroporation methodology may be used for delivery of any one of the nucleic acid components disclosed herein. In some embodiments, electroporation may be used to deliver a polynucleotide and optional one or more nucleic acid components.

In some embodiments, a method is provided for delivering a polynucleotide disclosed herein to an ex vivo cell, wherein the polynucleotide is associated with an LNP or not associated with an LNP. In some embodiments, the polynucleotide/LNP or polynucleotide is also associated with optional one or more nucleic acid components.

In some embodiments, a pharmaceutical formulation comprising a polynucleotide according to the disclosure is provided. In some embodiments, a pharmaceutical formulation comprising at least one lipid, for example, an LNP which comprises a polynucleotide according to the disclosure, is provided. Any LNP suitable for delivering a polynucleotide can be used, such as those described above; additional exemplary LNPs are described in WO2017173054A1 published Oct. 5, 2017. A pharmaceutical formulation can further comprise a pharmaceutically acceptable carrier, e.g., water or a buffer. A pharmaceutical formulation can further comprise one or more pharmaceutically acceptable excipients, such as a stabilizer, preservative, bulking agent, or the like. A pharmaceutical formulation can further comprise one or more pharmaceutically acceptable salts, such as sodium chloride. In some embodiments, the pharmaceutical formulation is formulated for intravenous administration. In some embodiments, the pharmaceutical formulation is formulated for delivery into the hepatic circulation.

### B. Determination of Efficacy of ORFs

The efficacy of a polynucleotide comprising an ORF encoding a polypeptide of interest may be determined when the polypeptide is expressed together with other components for a target function or system, e.g., using any of those recognized in the art to detect the presence, expression level, or activity of a particular polypeptide, e.g., by enzyme linked immunosorbent assay (ELISA), other immunological methods, Western blots), liquid chromatography-mass spectrometry (LC-MS), FACS analysis, or other assays described herein; or methods for determining enzymatic activity levels in biological samples (e.g., cell lysates or extracts, conditioned medium, whole blood, serum, plasma, urine, or tissue), such as in vitro activity assays. Exemplary assays for activity of various encoded polypeptides described herein include assays for phenylalanine hydroxylase enzymatic activity; ornithine carbamoyltransferase enzymatic activity; fumarylacetoacetate hydrolase enzymatic activity; glucosylceramidase beta enzymatic activity; alpha galactosidase enzymatic activity; a transthyretin; a glyceraldehyde-3-phosphate dehydrogenase enzymatic activity; serine protease inhibition; neurotransmitter binding (e.g., GABA binding). In some embodiments, the efficacy of a polynucleotide comprising an ORF encoding a polypeptide of interest is determined based on in vitro models.

### 1. Determination of Efficacy of ORFs Encoding an RNA-Guided DNA-Binding Agent

In some embodiments, the efficacy of an mRNA is determined when expressed together with other components of an RNP, e.g., at least one gRNA, such as a gRNA targeting TTR.

An RNA-guided DNA-binding agent with cleavase activity can lead to double-stranded breaks in the DNA. Nonhomologous end joining (NHEJ) is a process whereby double-stranded breaks (DSBs) in the DNA are repaired via re-ligation of the break ends, which can produce errors in the form of insertion/deletion (indel) mutations. The DNA ends of a DSB are frequently subjected to enzymatic processing, resulting in the addition or removal of nucleotides at one or both strands before the rejoining of the ends. These additions or removals prior to rejoining result in the presence of insertion or deletion (indel) mutations in the DNA sequence at the site of the NHEJ repair. Many mutations due to indels alter the reading frame or introduce premature stop codons and, therefore, produce a non-functional protein.

In some embodiments, the efficacy of an mRNA encoding a nuclease is determined based on in vitro models. In some embodiments, the in vitro model is HEK293 cells. In some embodiments, the in vitro model is HUH7 human hepatocarcinoma cells. In some embodiments, the in vitro model is primary hepatocytes, such as primary human or mouse hepatocytes.

In some embodiments, the efficacy of an RNA is measured by percent editing of TTR. Exemplary procedures for determining percent editing are given in the Examples below. In some embodiments, the percent editing of TTR is compared to the percent editing obtained when the mRNA comprises an ORF of SEQ ID NO: 2 or 3 with unmodified uridine and all else is equal.

In some embodiments, the efficacy of an mRNA is determined by measuring the protein expression levels, e.g. by an MSD technique or by quantifying a detectable marker linked to the protein. In some embodiments, the efficacy of an mRNA is determined using serum TTR concentration in a mouse following administration of an LNP comprising the mRNA and a gRNA targeting TTR, e.g., SEQ ID NO: 4. The serum TTR concentration can be expressed in absolute terms or in % knockdown relative to a sham-treated control. In some embodiments, the efficacy of an mRNA is determined using percentage editing in the liver in a mouse following administration of an LNP comprising the mRNA and a gRNA targeting TTR, e.g., SEQ ID NO: 4. In some embodiments, an effective amount is able to achieve at least 50% editing or 50% knockdown of serum TTR. Exemplary effective amounts are in the range of 0.1 to 10 mg/kg (mpk), e.g., 0.1 to 0.3 mpk, 0.3 to 0.5 mpk, 0.5 to 1 mpk, 1 to 2 mpk, 2 to 3 mpk, 3 to 5 mpk, 5 to 10 mpk, or 0.1, 0.2, 0.3, 0.5, 1, 2, 3, 5, or 10 mpk.

In some embodiments, detecting gene editing events, such as the formation of insertion/deletion (“indel”) mutations and homology directed repair (HDR) events in target DNA utilize linear amplification with a tagged primer and isolating the tagged amplification products (herein after referred to as “LAM-PCR,” or “Linear Amplification (LA)” method, as described in WO2018/067447 or Schmidt et al., Nature Methods 4:1051-1057 (2007), or next-generation sequencing (“NGS”; e.g., using the Illumina NGS platform) as described below or other methods known in the art for detecting indel mutations.

For example, to quantitatively determine the efficiency of editing at the target location in the genome, in the NGS method, genomic DNA is isolated and deep sequencing is utilized to identify the presence of insertions and deletions introduced by gene editing. PCR primers are designed around the target site (e.g., TTR), and the genomic area of interest is amplified. Additional PCR is performed according to the manufacturer's protocols (Illumina) to add the necessary chemistry for sequencing. The amplicons are sequenced on an Illumina MiSeq instrument. The reads are aligned to the reference genome (e.g., mm10) after eliminating those having low quality scores. The resulting files containing the reads are mapped to the reference genome (BAM files), where reads that overlapped the target region of interest are selected and the number of wild type reads versus the number of reads which contain an insertion, substitution, or deletion is calculated. The editing percentage (e.g., the “editing efficiency” or “percent editing”) is defined as the total number of sequence reads with insertions or deletions over the total number of sequence reads, including wild type.

### C. Exemplary Uses, Methods, and Treatments

In some embodiments, a polynucleotide, expression construct, composition, lipid nanoparticle (LNP), or pharmaceutical composition is for use in gene therapy, e.g. of a target gene. In some embodiments, a polynucleotide, expression construct, composition, lipid nanoparticle (LNP), or pharmaceutical composition is for use in genome editing, e.g., editing a target gene wherein the polynucleotide encodes an RNA-guided DNA binding agent. In some embodiments, a polynucleotide, expression construct, composition, lipid nanoparticle (LNP), or pharmaceutical composition disclosed herein encoding a polypeptide of interest is for use in expressing the polypeptide of interest in a heterologous cell, e.g., a human cell or a mouse cell. In some embodiments, a polynucleotide, expression construct, composition, lipid nanoparticle (LNP), or pharmaceutical composition is for use in modifying a target gene, e.g., altering its sequence or epigenetic status wherein the polynucleotide encodes an RNA-guided DNA binding agent. In some embodiments, a polynucleotide, expression construct, composition, lipid nanoparticle (LNP), or pharmaceutical composition is for use in inducing a double-stranded break (DSB) within a target gene. In some embodiments, a polynucleotide, expression construct, composition, lipid nanoparticle (LNP), or pharmaceutical composition is for use in inducing an indel within a target gene. In some embodiments, the use of a polynucleotide, expression construct, composition, lipid nanoparticle (LNP), or pharmaceutical composition disclosed herein is provided for the preparation of a medicament for genome editing, e.g., editing a target gene wherein the polynucleotide encodes an RNA-guided DNA binding agent. In some embodiments, the use of a polynucleotide, expression construct, composition, lipid nanoparticle (LNP), or pharmaceutical composition disclosed herein encoding a polypeptide of interest is provided for the preparation of a medicament for expressing the polypeptide of interest in a heterologous cell or increasing the expression of the polypeptide of interest, e.g., a human cell or a mouse cell. In some embodiments, the use of a polynucleotide, expression construct, composition, lipid nanoparticle (LNP), or pharmaceutical composition disclosed herein is provided for the preparation of a medicament for modifying a target gene, e.g., altering its sequence or epigenetic status. In some embodiments, the use of a polynucleotide, expression construct, composition, lipid nanoparticle (LNP), or pharmaceutical composition disclosed herein is provided for the preparation of a medicament for inducing a double-stranded break (DSB) within a target gene. In some embodiments, the use of a polynucleotide, expression construct, composition, lipid nanoparticle (LNP), or pharmaceutical composition disclosed herein is provided for the preparation of a medicament for inducing an indel within a target gene.

In some embodiments, the target gene is a transgene. In some embodiments, the target gene is an endogenous gene. The target gene may be in a subject, such as a mammal, such as a human. In some embodiments, the target gene is in an organ, such as a liver, such as a mammalian liver, such as a human liver. In some embodiments, the target gene is in a liver cell, such as a mammalian liver cell, such as a human liver cell. In some embodiments, the target gene is in a hepatocyte, such as a mammalian hepatocyte, such as a human hepatocyte. In some embodiments, the liver cell or hepatocyte is in situ. In some embodiments, the liver cell or hepatocyte is isolated, e.g., in a culture, such as in a primary culture.

Also provided are methods corresponding to the uses disclosed herein, which comprise administering the polynucleotide, expression construct, composition, lipid nanoparticle (LNP), or pharmaceutical composition disclosed herein to a subject or contacting a cell such as those described above with the polynucleotide, LNP, or pharmaceutical composition disclosed herein, e.g., to express a polypeptide of interest or increase the expression of a polypeptide of interest, e.g., in a heterologous cell, such as a human cell or a mouse cell.

In some embodiments, a polynucleotide, expression construct, composition, lipid nanoparticle (LNP), or pharmaceutical composition is for use in therapy or in treating a disease, e.g., amyloidosis associated with TTR (ATTR) or alpha-1 anti-trypsin disorder; phenylketonuria (PKU) or phenylalanine hydroxylase deficiency; ornithine carbamoyltransferase (OTC) deficiency or hyperammonemia; glucosylceramidase deficiency or Glucocerebrosidosis or Gaucher disease; alpha-galactosidase A (GLA) deficiency or Fabry disease; fumarylacetoacetase (FAH) deficiency or Trosinemia type I. In some instances, the disease is associated with the ORF or polypeptide of interest. In some embodiments, the use of a polynucleotide disclosed herein (e.g., in a composition provided herein) is provided for the preparation of a medicament, e.g., for treating a subject having amyloidosis associated with TTR (ATTR); alpha-1 anti-trypsin disorder; phenylketonuria (PKU) or phenylalanine hydroxylase deficiency; ornithine carbamoyltransferase (OTC) deficiency or hyperammonemia; glucosylceramidase deficiency or Glucocerebrosidosis or Gaucher disease; alpha-galactosidase A (GLA) deficiency or Fabry disease; fumarylacetoacetase (FAH) deficiency or Trosinemia type I.

In some embodiments, a polynucleotide, expression construct, composition, lipid nanoparticle (LNP), or pharmaceutical composition is administered intravenously for any of the uses discussed above concerning organisms, organs, or cells in situ. In some embodiments, a polynucleotide, expression construct, composition, lipid nanoparticle (LNP), or pharmaceutical composition is administered at a dose in the range of 0.01 to 10 mg/kg (mpk), e.g., 0.01 to 0.1 mpk, 0.1 to 0.3 mpk, 0.3 to 0.5 mpk, 0.5 to 1 mpk, 1 to 2 mpk, 2 to 3 mpk, 3 to 5 mpk, 5 to 10 mpk, or 0.1, 0.2, 0.3, 0.5, 1, 2, 3, 5, or 10 mpk.

In any of the foregoing embodiments involving a subject, the subject can be mammalian. In any of the foregoing embodiments involving a subject, the subject can be human. In any of the foregoing embodiments involving a subject, the subject can be a cow, pig, monkey, sheep, dog, cat, fish, or poultry.

In some embodiments, a polynucleotide, expression construct, composition, lipid nanoparticle (LNP), or pharmaceutical composition disclosed herein is administered intravenously or for intravenous administration. In some embodiments, a polynucleotide, LNP, or pharmaceutical composition disclosed herein are administered into the hepatic circulation or for administration into the hepatic circulation.

In some embodiments, a single administration of a polynucleotide, LNP, or pharmaceutical composition disclosed herein is sufficient to knock down expression of the target gene product. In some embodiments, a single administration of a polynucleotide, LNP, or pharmaceutical composition disclosed herein is sufficient to knock out expression of the target gene product. In other embodiments, more than one administration of a polynucleotide, LNP, or pharmaceutical composition disclosed herein may be beneficial to maximize editing, modification, indel formation, DSB formation, or the like via cumulative effects.

In some embodiments, the efficacy of treatment with a polynucleotide, LNP, or pharmaceutical composition disclosed herein is seen at 1 year, 2 years, 3 years, 4 years, 5 years, or 10 years after delivery.

In some embodiments, treatment slows or halts disease progression.

In some embodiments, treatment results in improvement, stabilization, or slowing of change in organ function or symptoms of disease of an organ, such as the liver.

In some embodiments, efficacy of treatment is measured by increased survival time of the subject.

### D. Exemplary DNA Molecules, Vectors, Expression Constructs, Host Cells, and Production Methods

In certain embodiments, the disclosure provides a DNA molecule comprising a sequence encoding an ORF encoding a polypeptide of interest. In some embodiments, in addition to the ORF sequence sequences, the DNA molecule further comprises nucleic acids that do not encode the polypeptide. Nucleic acids that do not encode the polypeptide include, but are not limited to, promoters, enhancers, regulatory sequences, and nucleic acids encoding a guide RNA.

In some embodiments, the DNA molecule further comprises a nucleotide sequence encoding a crRNA, a trRNA, or a crRNA and trRNA. In some embodiments, the nucleotide sequence encoding the crRNA, trRNA, or crRNA and trRNA comprises or consists of a guide sequence flanked by all or a portion of a repeat sequence from a naturally-occurring CRISPR/Cas system. The nucleic acid comprising or consisting of the crRNA, trRNA, or crRNA and trRNA may further comprise a vector sequence wherein the vector sequence comprises or consists of nucleic acids that are not naturally found together with the crRNA, trRNA, or crRNA and trRNA. In some embodiments, the crRNA and the trRNA are encoded by non-contiguous nucleic acids within one vector. In other embodiments, the crRNA and the trRNA may be encoded by a contiguous nucleic acid. In some embodiments, the crRNA and the trRNA are encoded by opposite strands of a single nucleic acid. In other embodiments, the crRNA and the trRNA are encoded by the same strand of a single nucleic acid.

In some embodiments, the DNA molecule further comprises a promoter operably linked to the sequence encoding any of the ORF encoding a polypeptide of interest. In some embodiments, the DNA molecule is an expression construct suitable for expression in a mammalian cell, e.g., a human cell or a mouse cell, such as a human hepatocyte or a rodent (e.g., mouse) hepatocyte. In some embodiments, the DNA molecule is an expression construct suitable for expression in a cell of a mammalian organ, e.g., a human liver or a rodent (e.g., mouse) liver. In some embodiments, the DNA molecule is a plasmid or an episome. In some embodiments, the DNA molecule is contained in a host cell, such as a bacterium or a cultured eukaryotic cell. Exemplary bacteria include proteobacteria such as E. coli. Exemplary cultured eukaryotic cells include primary hepatocytes, including hepatocytes of rodent (e.g., mouse) or human origin; hepatocyte cell lines, including hepatocytes of rodent (e.g., mouse) or human origin; human cell lines; rodent (e.g., mouse) cell lines; CHO cells; microbial fungi, such as fission or budding yeasts, e.g., Saccharomyces, such as S. cerevisiae; and insect cells.

In some embodiments, a method of producing an mRNA disclosed herein is provided. In some embodiments, such a method comprises contacting a DNA molecule described herein with an RNA polymerase under conditions permissive for transcription. In some embodiments, the contacting is performed in vitro, e.g., in a cell-free system. In some embodiments, the RNA polymerase is an RNA polymerase of bacteriophage origin, such as T7 RNA polymerase. In some embodiments, NTPs are provided that include at least one modified nucleotide as discussed above. In some embodiments, the NTPs include at least one modified nucleotide as discussed above and do not comprise UTP.

In some embodiments, a polynucleotide disclosed herein may be comprised within or delivered by a vector system of one or more vectors. In some embodiments, one or more of the vectors, or all of the vectors, may be DNA vectors. In some embodiments, one or more of the vectors, or all of the vectors, may be RNA vectors. In some embodiments, one or more of the vectors, or all of the vectors, may be circular. In other embodiments, one or more of the vectors, or all of the vectors, may be linear. In some embodiments, one or more of the vectors, or all of the vectors, may be enclosed in a lipid nanoparticle, liposome, non-lipid nanoparticle, or viral capsid. Non-limiting exemplary vectors include plasmids, phagemids, cosmids, artificial chromosomes, minichromosomes, transposons, viral vectors, and expression vectors.

Non-limiting exemplary viral vectors include adeno-associated virus (AAV) vector, lentivirus vectors, adenovirus vectors, helper dependent adenoviral vectors (HDAd), herpes simplex virus (HSV-1) vectors, bacteriophage T4, baculovirus vectors, and retrovirus vectors. In some embodiments, the viral vector may be an AAV vector. In other embodiments, the viral vector may a lentivirus vector. In some embodiments, the lentivirus may be non-integrating. In some embodiments, the viral vector may be an adenovirus vector. In some embodiments, the adenovirus may be a high-cloning capacity or “gutless” adenovirus, where all coding viral regions apart from the 5′ and 3′ inverted terminal repeats (ITRs) and the packaging signal (‘I’) are deleted from the virus to increase its packaging capacity. In yet other embodiments, the viral vector may be an HSV-1 vector. In some embodiments, the HSV-1-based vector is helper dependent, and in other embodiments it is helper independent. For example, an amplicon vector that retains only the packaging sequence requires a helper virus with structural components for packaging, while a 30 kb-deleted HSV-1 vector that removes non-essential viral functions does not require helper virus. In additional embodiments, the viral vector may be bacteriophage T4. In some embodiments, the bacteriophage T4 may be able to package any linear or circular DNA or RNA molecules when the head of the virus is emptied. In further embodiments, the viral vector may be a baculovirus vector. In yet further embodiments, the viral vector may be a retrovirus vector. In embodiments using AAV or lentiviral vectors, which have smaller cloning capacity, it may be necessary to use more than one vector to deliver all the components of a vector system as disclosed herein. For example, one AAV vector may contain sequences encoding a Cas protein, while a second AAV vector may contain one or more guide sequences.

In some embodiments, the vector may be capable of driving expression of one or more coding sequences, such as the coding sequence of an mRNA disclosed herein, in a cell. In some embodiments, the cell may be a prokaryotic cell, such as, e.g., a bacterial cell. In some embodiments, the cell may be a eukaryotic cell, such as, e.g., a yeast, plant, insect, or mammalian cell. In some embodiments, the eukaryotic cell may be a mammalian cell. In some embodiments, the eukaryotic cell may be a rodent cell. In some embodiments, the eukaryotic cell may be a human cell. Suitable promoters to drive expression in different types of cells are known in the art. In some embodiments, the promoter may be wild type. In other embodiments, the promoter may be modified for more efficient or efficacious expression. In yet other embodiments, the promoter may be truncated yet retain its function. For example, the promoter may have a normal size or a reduced size that is suitable for proper packaging of the vector into a virus.

In some embodiments, the vector system may comprise one copy of a nucleotide sequence encoding an ORF a polypeptide of interest. In other embodiments, the vector system may comprise more than one copy of a nucleotide sequence encoding a polypeptide of interest. In some embodiments, the nucleotide sequence encoding the polypeptide of interest may be operably linked to at least one transcriptional or translational control sequence. In some embodiments, the nucleotide sequence encoding the nuclease may be operably linked to at least one promoter.

In some embodiments, the promoter may be constitutive, inducible, or tissue-specific. In some embodiments, the promoter may be a constitutive promoter. Non-limiting exemplary constitutive promoters include cytomegalovirus immediate early promoter (CMV), simian virus (SV40) promoter, adenovirus major late (MLP) promoter, Rous sarcoma virus (RSV) promoter, mouse mammary tumor virus (MMTV) promoter, phosphoglycerate kinase (PGK) promoter, elongation factor-alpha (EF1a) promoter, ubiquitin promoters, actin promoters, tubulin promoters, immunoglobulin promoters, a functional fragment thereof, or a combination of any of the foregoing. In some embodiments, the promoter may be a CMV promoter. In some embodiments, the promoter may be a truncated CMV promoter. In other embodiments, the promoter may be an EF1a promoter. In some embodiments, the promoter may be an inducible promoter. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech).

In some embodiments, the promoter may be a tissue-specific promoter, e.g., a promoter specific for expression in the liver.

The vector may further comprise a nucleotide sequence encoding at least one guide RNA. In some embodiments, the vector comprises one copy of the guide RNA. In other embodiments, the vector comprises more than one copy of the guide RNA. In embodiments with more than one guide RNA, the guide RNAs may be non-identical such that they target different target sequences, or may be identical in that they target the same target sequence. In some embodiments where the vectors comprise more than one guide RNA, each guide RNA may have other different properties, such as activity or stability within a ribonucleoprotein complex with the RNA-guided DNA-binding agent. In some embodiments, the nucleotide sequence encoding the guide RNA may be operably linked to at least one transcriptional or translational control sequence, such as a promoter, a 3′ UTR, or a 5′ UTR. In one embodiment, the promoter may be a tRNA promoter, e.g., tRNA^{Lys3}, or a tRNA chimera. See Mefferd et al., RNA. 2015 21:1683-9; Scherer et al., Nucleic Acids Res. 2007 35: 2620-2628. In some embodiments, the promoter may be recognized by RNA polymerase III (Pol III). Non-limiting examples of Pol III promoters include U6 and H1 promoters. In some embodiments, the nucleotide sequence encoding the guide RNA may be operably linked to a mouse or human U6 promoter. In other embodiments, the nucleotide sequence encoding the guide RNA may be operably linked to a mouse or human H1 promoter. In embodiments with more than one guide RNA, the promoters used to drive expression may be the same or different. In some embodiments, the nucleotide encoding the crRNA of the guide RNA and the nucleotide encoding the trRNA of the guide RNA may be provided on the same vector. In some embodiments, the nucleotide encoding the crRNA and the nucleotide encoding the trRNA may be driven by the same promoter. In some embodiments, the crRNA and trRNA may be transcribed into a single transcript. For example, the crRNA and trRNA may be processed from the single transcript to form a double-molecule guide RNA. Alternatively, the crRNA and trRNA may be transcribed into a single-molecule guide RNA. In other embodiments, the crRNA and the trRNA may be driven by their corresponding promoters on the same vector. In yet other embodiments, the crRNA and the trRNA may be encoded by different vectors.

In some embodiments, the compositions comprise a vector system, wherein the system comprises more than one vector. In some embodiments, the vector system may comprise one single vector. In other embodiments, the vector system may comprise two vectors. In additional embodiments, the vector system may comprise three vectors. When different polynucleotides are used for multiplexing, or when multiple copies of the polynucleotides are used, the vector system may comprise more than three vectors.

In some embodiments, the vector system may comprise inducible promoters to start expression only after it is delivered to a target cell. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech).

In additional embodiments, the vector system may comprise tissue-specific promoters to start expression only after it is delivered into a specific tissue.

### EXAMPLES

The following examples are provided to illustrate certain disclosed embodiments and are not to be construed as limiting the scope of this disclosure in any way.

### Example 1—General Reagents and Methods

### LNP Formulation

The lipid components were dissolved in 100% ethanol with the lipid component molar ratios described below. The chemically modified sgRNA and Cas9 mRNA were combined and dissolved in 25 mM citrate, 100 mM NaCl, pH 5.0, resulting in a concentration of total RNA cargo of approximately 0.45 mg/mL. The LNPs were formulated with an N/P ratio of about 6, with the ratio of chemically modified sgRNA: Cas9 mRNA at either a 1:1 or 1:2 w/w ratio as described below. Unless otherwise indicated, LNPs were formulated with 50% Lipid A, 9% DSPC, 38% cholesterol, and 3% PEG2k-DMG.

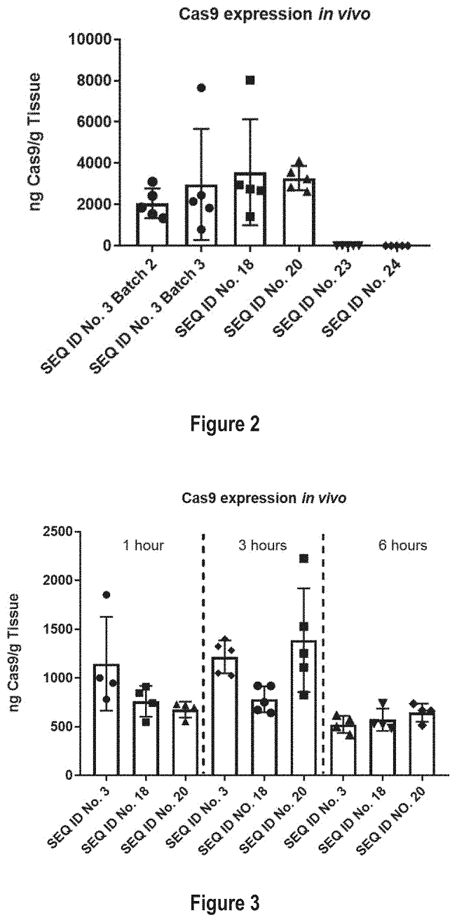

The LNPs were formed by an impinging jet mixing of the lipid in ethanol with two volumes of RNA solution and one volume of water. The lipid in ethanol is mixed through a mixing cross with the two volumes of RNA solution. A fourth stream of water is mixed with the outlet stream of the cross through an inline tee. (See, e.g., WO2016010840, FIG. 2.) A 2:1 ratio of aqueous to organic solvent was maintained during mixing using differential flow rates. The LNPs were held for 1 hour at room temperature, and further diluted with water (approximately 1:1 v/v). Diluted LNPs were concentrated using tangential flow filtration on a flat sheet cartridge (Sartorius, 100 kD MWCO) and then buffer exchanged by diafiltration into 50 mM Tris, 45 mM NaCl, 5% (w/v) sucrose, pH 7.5 (TSS). Alternatively, the final buffer exchange into TSS was completed with PD-10 desalting columns (GE). If required, compositions were concentrated by centrifugation with Amicon 100 kDa centrifugal filters (Millipore). The resulting mixture was then filtered using a 0.2 μm sterile filter. The final LNP was stored at 4° C. or −80° C. until further use.

### LNP Composition Analytics

Dynamic Light Scattering (“DLS”) is used to characterize the polydispersity index (“pdi”) and size of the LNPs of the present disclosure. DLS measures the scattering of light that results from subjecting a sample to a light source. PDI, as determined from DLS measurements, represents the distribution of particle size (around the mean particle size) in a population, with a perfectly uniform population having a PDI of zero.

Electrophoretic light scattering is used to characterize the surface charge of the LNP at a specified pH. The surface charge, or the zeta potential, is a measure of the magnitude of electrostatic repulsion/attraction between particles in the LNP suspension.

Asymetric-Flow Field Flow Fractionation—Multi-Angle Light Scattering (AF4-MALS) is used to separate particles in the composition by hydrodynamic radius and then measure the molecular weights, hydrodynamic radii and root mean square radii of the fractionated particles. This allows the ability to assess molecular weight and size distributions as well as secondary characteristics such as the Burchard-Stockmeyer Plot (ratio of root mean square (“rms”) radius to hydrodynamic radius over time suggesting the internal core density of a particle) and the rms conformation plot (log of rms radius vs log of molecular weight where the slope of the resulting linear fit gives a degree of compactness vs elongation).

Nanoparticle tracking analysis (NTA, Malvern Nanosight) can be used to determine particle size distribution as well as particle concentration. LNP samples are diluted appropriately and injected onto a microscope slide. A camera records the scattered light as the particles are slowly infused through field of view. After the movie is captured, the Nanoparticle Tracking Analysis processes the movie by tracking pixels and calculating a diffusion coefficient. This diffusion coefficient can be translated into the hydrodynamic radius of the particle. The instrument also counts the number of individual particles counted in the analysis to give particle concentration.

Cryo-electron microscopy (“cryo-EM”) can be used to determine the particle size, morphology, and structural characteristics of an LNP.

Lipid compositional analysis of the LNPs can be determined from liquid chromatography followed by charged aerosol detection (LC-CAD). This analysis can provide a comparison of the actual lipid content versus the theoretical lipid content.

LNP compositions are analyzed for average particle size, polydispersity index (pdi), total RNA content, encapsulation efficiency of RNA, and zeta potential. LNP compositions may be further characterized by lipid analysis, AF4-MALS, NTA, and/or cryo-EM. Average particle size and polydispersity are measured by dynamic light scattering (DLS) using a Malvern Zetasizer DLS instrument. LNP samples were diluted with PBS buffer prior to being measured by DLS. Z-average diameter which is an intensity-based measurement of average particle size is reported along with number average diameter and pdi. A Malvern Zetasizer instrument is also used to measure the zeta potential of the LNP. Samples are diluted 1:17 (50 μL into 800 μL) in 0.1×PBS, pH 7.4 prior to measurement.

A fluorescence-based assay (Ribogreen®, ThermoFisher Scientific) is used to determine total RNA concentration and free RNA. Encapsulation efficiency is calculated as (Total RNA—Free RNA)/Total RNA. LNP samples are diluted appropriately with 1×TE buffer containing 0.2% Triton-X 100 to determine total RNA or 1×TE buffer to determine free RNA. Standard curves are prepared by utilizing the starting RNA solution used to make the compositions and diluted in 1×TE buffer +/−0.2% Triton-X 100. Diluted RiboGreen® dye (according to the manufacturer's instructions) is then added to each of the standards and samples and allowed to incubate for approximately 10 minutes at room temperature, in the absence of light. A SpectraMax M5 Microplate Reader (Molecular Devices) is used to read the samples with excitation, auto cutoff and emission wavelengths set to 488 nm, 515 nm, and 525 nm respectively. Total RNA and free RNA are determined from the appropriate standard curves.

Encapsulation efficiency is calculated as (Total RNA—Free RNA)/Total RNA. The same procedure may be used for determining the encapsulation efficiency of a DNA-based cargo component. In a fluorescence-based assay, for single-strand DNA Oligreen Dye may be used, and for double-strand DNA, Picogreen Dye. Alternatively, the total RNA concentration can be determined by a reverse-phase ion-pairing (RP-IP) HPLC method. Triton X-100 is used to disrupt the LNPs, releasing the RNA. The RNA is then separated from the lipid components chromatographically by RP-IP HPLC and quantified against a standard curve using UV absorbance at 260 nm.

AF4-MALS is used to look at molecular weight and size distributions as well as secondary statistics from those calculations. LNPs are diluted as appropriate and injected into a AF4 separation channel using an HPLC autosampler where they are focused and then eluted with an exponential gradient in cross flow across the channel. All fluid is driven by an HPLC pump and Wyatt Eclipse Instrument. Particles eluting from the AF4 channel flow through a UV detector, multi-angle light scattering detector, quasi-elastic light scattering detector and differential refractive index detector. Raw data is processed by using a Debeye model to determine molecular weight and rms radius from the detector signals.

Lipid components in LNPs are analyzed quantitatively by HPLC coupled to a charged aerosol detector (CAD). Chromatographic separation of 4 lipid components is achieved by reverse phase HPLC. CAD is a destructive mass-based detector which detects all non-volatile compounds and the signal is consistent regardless of analyte structure.

mRNA and gRNA Production

Capped and polyadenylated mRNA was generated by in vitro transcription using a linearized plasmid DNA template and T7 RNA polymerase. Generally, plasmid DNA containing a T7 promoter and a poly(A/T) region between 90-100 nt is linearized by incubating at 37° C. with XbaI to completion. The linearized plasmid is purified from enzyme and buffer salts. The IVT reaction to generate Cas9 modified mRNA is performed by incubating at 37° C. for 1.5 or 2 hours in the following conditions: 50 ng/μL linearized plasmid; 5 mM each of GTP, ATP, CTP, and N1-methyl pseudo-UTP (Trilink); 25 mM ARCA (Trilink); 5 U/μL T7 RNA polymerase; 1 U/μL Murine RNase inhibitor; 0.004 U/μL Inorganic E. coli pyrophosphatase; and 1× reaction buffer. TURBO DNase (ThermoFisher) is then added to remove the DNA template.

mRNA is purified from enzyme and nucleotides using a RNeasy Maxi kit (Qiagen) according to the manufacturer's protocol. Alternately, mRNA is purified using a MEGAclear kit (Invitrogen) according to the manufacturer's protocol. Alternatively, mRNA is purified using LiCl precipitation, ammonium acetate precipitation and sodium acetate precipitation. Alternatively, mRNA is purified with a LiCl precipitation method followed by further purification by tangential flow filtration. Alternatively, RNA was purified by LiCl precipitation in combination with tangential flow filtration. The transcript concentration was determined by measuring the light absorbance at 260 nm (Nanodrop), and the transcript was analyzed by capillary electrophoresis by Fragment Analyzer (Agilent).

The sgRNA was chemically synthesized by known methods using phosphoramidites.

Cas9 mRNA and Guide RNA Delivery to Primary Hepatocytes In Vitro

Primary mouse hepatocytes (PMH) and primary cyno hepatocytes (PCH) were thawed and resuspended in hepatocyte thawing medium with supplements (Invitrogen, Cat. CM7000) followed by centrifugation. The supernatant was discarded, and the pelleted cells resuspended in William's Medium E (Gibco, Cat. A12176) plating medium plus supplement pack (Gibco, Cat. A15563) and 5% FBS (Gibco). Cells were counted and plated on Bio-coat collagen I coated 96-well plates (ThermoFisher, Cat. 877272) at a density of 50,000 cells/well for PCH and 15,000 cells/well for PMH. Plated cells were allowed to settle and adhere for 5 hours in a tissue culture incubator at 37° C. and 5% CO₂ atmosphere. After incubation cells were checked for monolayer formation and were washed three times with William's Medium E with cell maintenance supplements (Gibco, Cat. A15564) prior and incubated at 37° C. incubator.

PMH and PCH were transfected with 200 ng of mRNA using 0.6 or 0.3 ul of MessengerMAX per well for PMH and PCH, respectively. Transfections were carried out according to manufacturer's protocol (ThermoFisher Scientific, Cat #LMRN003). Media was collected 6, 24, and 48 hours post-treatment to assay for hA1AT expression.

Genomic DNA was extracted from each well of a 96-well plate using 50 μL/well BuccalAmp DNA Extraction solution (Epicentre, Cat. QE09050) according to manufacturer's protocol. All DNA samples were subjected to PCR and subsequent NGS analysis, as described herein.

### LNP Delivery In Vivo

CD-1 female mice, ranging from 6-10 weeks of age were used in each study. Animals were weighed and grouped according to body weight for preparing dosing solutions based on group average weight. LNPs were dosed via the lateral tail vein in a volume of 0.2 mL per animal (approximately 10 mL per kilogram body weight). Animals were euthanized at 6 or 7 days by exsanguination via cardiac puncture under isoflurane anesthesia. Blood, if needed, was collected into serum separator tubes or into tubes containing buffered sodium citrate for plasma as described herein. For studies involving in vivo editing or protein level measurements, liver tissue was collected from each animal for DNA or protein extraction and analysis. Cohorts of mice were measured for liver editing by Next-Generation Sequencing (NGS). For Cas9 protein analysis, approximately 30-80 mg liver tissue was homogenized by bead mill in RIPA Buffer (Boston Bioproducts BP-115) with 1× Complete Protease Inhibitor Tablet (Roche, Cat.11836170001).

### NGS Sequencing

In brief, to quantitatively determine the efficiency of editing at the target location in the genome, genomic DNA was isolated and deep sequencing was utilized to identify the presence of insertions and deletions introduced by gene editing.

PCR primers were designed around the target site (e.g., TTR), and the genomic area of interest was amplified. Primer sequences are provided below. Additional PCR was performed according to the manufacturer's protocols (Illumina) to add the necessary chemistry for sequencing. The amplicons were sequenced on an Illumina MiSeq instrument. The reads were aligned to the reference genome (e.g., mm10) after eliminating those having low quality scores. The resulting files containing the reads were mapped to the reference genome (BAM files), where reads that overlapped the target region of interest were selected and the number of wild type reads versus the number of reads which contain an insertion, substitution, or deletion was calculated.

The editing percentage (e.g., the “editing efficiency” or “percent editing”) is defined as the total number of sequence reads with insertions or deletions over the total number of sequence reads, including wild type.

### Cas9 Protein Measurement

Cas9 protein levels were determined by ELISA assay. Briefly, total protein concentration are optionally determined by bicinchoninic acid assay. An MSD GOLD 96-well Streptavidin SECTOR Plate (Meso Scale Diagnostics, Cat. L15SA-1) was prepared according to manufacturer's protocol using Cas9 mouse antibody (Origene, Cat. CF811179) as the capture antibody and Cas9 (7A9-3A3) Mouse mAb (Cell Signaling Technology, Cat. 14697) as the detection antibody. Recombinant Cas9 protein was used as a calibration standard in Diluent 39 (Meso Scale Diagnostics) with 1×Halt™ Protease Inhibitor Cocktail, EDTA-Free (ThermoFisher, Cat. 78437). ELISA plates were read using the Meso Quickplex SQ120 instrument (Meso Scale Discovery) and data was analyzed with Discovery Workbench 4.0 software package (Meso Scale Discovery).

### Serum TTR Measurement

The total mouse TTR serum levels were determined using a Mouse Prealbumin (Transthyretin) ELISA Kit (Aviva Systems Biology, Cat. OKIA00111). Briefly, sera were serial diluted with kit sample diluent to a final dilution of 10,000-fold for 0.1 mpk dose and 2,500-fold for 0.3 mpk. This diluted sample was then added to the ELISA plates and the assay was then carried out according to directions.

Human Alpha 1-Antitrypsin (hA1AT) Measurement

Human hA1AT levels were measured from media for in vitro studies. The total human alpha 1-antitripsin levels were determined using a Alpha 1-Antitrypsin ELISA Kit (Human) (Aviva Biosystems, Cat #OKIA00048) according to manufacturer's protocol. Serum hA1AT levels were quantitated off a standard curve using 4 parameter logistic fit and expressed as μg/mL of serum.

### Example 2—Characterization of Cas9 Expression In Vitro

Cas9 sequences using different codon schemes as described in Table 8 were designed to test for improved protein expression. Specifically, SEQ ID No: 3 and SEQ ID Nos: 15, 16, 17, 18, 19, 20, 21, 22, 23 and 24, comprising the ORFs of SEQ ID Nos. 5, 6, 7, 8, 9, 10, 11, 12, 13, and 14, respectively, were tested.

Translation efficiency was assessed in vitro by transfection of mRNA into HepG2 cells and measuring Cas9 protein expression levels by ELISA. HepG2 cells were transfected with 800 ng of each Cas9 mRNA using Lipofectamine™ MessengerMAX™ Transfection Reagent (ThermoFisher). Post transfection, cells were lysed by freeze thaw and cleared by centrifugation.

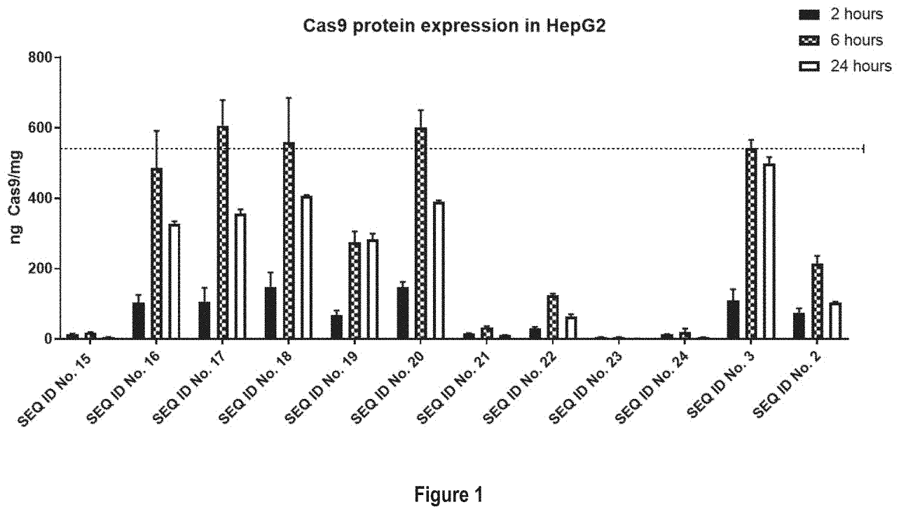

Two, six, or twenty-four hours post transfection, cells were lysed by freeze thaw and cleared by centrifugation. Cas9 protein expression was measured in these samples using the Meso Scale Discovery ELISA assay described in Example 1. Table 12 and FIG. 1 show the effects of the different codon schemes on Cas9 protein expression.

TABLE 12
In vitro expression of ORFs with different codon sets.

| | 2 hrs | | | 6 hrs | | | 24 hrs | | |
|---|---|---|---|---|---|---|---|---|---|
| | Mean (ng | | Fold | Mean (ng | | Fold | Mean (ng | | Fold |
| mRNA | Cas9/mg) | SD | change | Cas9/mg) | SD | change | Cas9/mg) | SD | change |
| SEQ ID No. 2 | 75 | 12 | 0.68 | 214 | 22 | 0.39 | 103 | 2 | 0.21 |
| SEQ ID No. 3 | 110 | 31 | — | 543 | 22 | — | 499 | 18 | — |
| SEQ ID No. 15 | 14 | 1 | 0.13 | 19 | 1 | 0.03 | 5 | 0 | 0.01 |
| SEQ ID No. 16 | 103 | 21 | 0.94 | 486 | 106 | 0.89 | 328 | 6 | 0.66 |
| SEQ ID No. 17 | 107 | 39 | 0.97 | 606 | 72 | 1.12 | 357 | 12 | 0.72 |
| SEQ ID No. 18 | 148 | 41 | 1.34 | 559 | 126 | 1.03 | 406 | 3 | 0.82 |
| SEQ ID No. 19 | 69 | 12 | 0.62 | 274 | 31 | 0.5 | 282 | 17 | 0.57 |
| SEQ ID No. 20 | 147 | 16 | 1.33 | 602 | 49 | 1.11 | 389 | 4 | 0.78 |
| SEQ ID No. 21 | 16 | 1 | 0.14 | 34 | 3 | 0.06 | 11 | 0 | 0.02 |
| SEQ ID No. 22 | 30 | 4 | 0.28 | 124 | 5 | 0.23 | 63 | 7 | 0.13 |
| SEQ ID No. 23 | 5 | 0 | 0.05 | 4 | 1 | 0.01 | 1 | 0 | 0 |
| SEQ ID No. 24 | 13 | 0 | 0.12 | 20 | 9 | 0.04 | 5 | 0 | 0.01 |

### Example 3—Characterization of Cas9 Expression In Vivo

To determine the effectiveness of the codon schemes in vivo, Cas9 protein expression was measured when expressed in vivo from mRNAs encoding Cas9 using codon schemes described in Table 8. Messenger RNAs were produced and formulated with a 1:2 w/w ratio of chemically modified sgRNA:Cas9 mRNA as described in Example 1. The LNPs contained a guide RNA targeting TTR (G000502; SEQ ID NO: 4). CD-1 female mice (n=5 per group) were dosed i.v. at 0.3 mpk. At 3 hours post-dose, animals were sacrificed, the liver was collected. Cas9 protein expression was measured in the liver using the Meso Scale Discovery ELISA assay described in Example 1. TABLE 13 and FIG. 2 show Cas9 expression results in liver. Cas9 mRNAs SEQ ID NOs: 18 and 20 showed the highest Cas9 expression of the tested ORFs and improved expression compared to other tested ORFs (SEQ ID NO: 3). Cas9 protein expression of the ORF of SEQ ID NOs: 23 and 24 were below the lower limit of quantitation (LLOQ).

TABLE 13
Cas 9 protein expression in liver

| | Mean | | Fold |
|---|---|---|---|
| mRNA | (ng Cas9/g Tissue) | SD | Improvement |
| SEQ ID No. 3 | 2972 | 2691 | 1 |
| Batch 3 | | | |
| SEQ ID No. 3 | 2053 | 718 | 0.6 |
| Batch 2 | | | |
| SEQ ID No. 18 | 3563 | 2568 | 1.2 |
| SEQ ID No. 20 | 3278 | 591 | 1.1 |
| SEQ ID No. 23 | Below LLOQ | — | N.D. |
| SEQ ID No. 24 | Below LLOQ | — | N.D. |

### Example 4—Time Course of Cas9 Protein Expression In Vivo

The durability of Cas9 protein expression from SEQ ID NO: 18 and SEQ ID No. 20 was assessed at various times after administration. Messenger RNAs were produced and formulated with a 1:2 w/w ratio of chemically modified sgRNA:Cas9 mRNA as described in Example 1. The LNPs contained a guide RNA targeting TTR (G000502; SEQ ID NO: 4). The CD-1 female mice (n=5 or n=4 per group, TABLE 14) were dosed i.v. at 0.3 mpk. At one, three, and six hours post-dose, animals were sacrificed, the liver was collected. Cas9 protein expression was measured in the liver samples using the Meso Scale Discovery ELISA assay described in Example 1. TABLE 14 and FIG. 3 show Cas9 expression results in liver. SEQ ID No. 20 showed the highest Cas9 expression of the tested ORFs at 3 and 6 hours post transfection and improved expression compared to other tested Cas9 ORFs.

TABLE 14
Time course of Cas9 protein expression in vivo

| | | Mean | | | Dose |
|---|---|---|---|---|---|
| mRNA | Time Point | (ng Cas9/g Tissue) | SD | n | (mg/kg) |
| SEQ ID No. 3 | 1 hr | 1145 | 480 | 4 | 0.3 |
| SEQ ID No. 18 | 1 hr | 760 | 159 | 4 | 0.3 |
| SEQ ID No. 20 | 1 hr | 715 | 757 | 4 | 0.3 |
| SEQ ID No. 2 | 3 hr | 613 | 165 | 5 | 0.3 |
| SEQ ID No. 3 | 3 hr | 1215 | 169 | 5 | 0.3 |
| SEQ ID No. 18 | 3 hr | 780 | 133 | 5 | 0.3 |
| SEQ ID No. 20 | 3 hr | 1387 | 532 | 5 | 0.3 |
| SEQ ID No. 3 | 6 hr | 523 | 88 | 4 | 0.3 |
| SEQ ID No. 18 | 6 hr | 571 | 114 | 4 | 0.3 |
| SEQ ID No. 20 | 6 hr | 644 | 93 | 4 | 0.3 |

### Example 5—Dose Response of Cas9 Protein Expression In Vivo

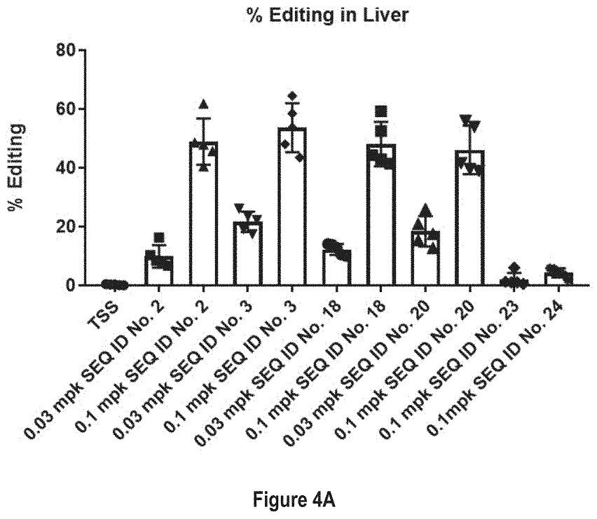

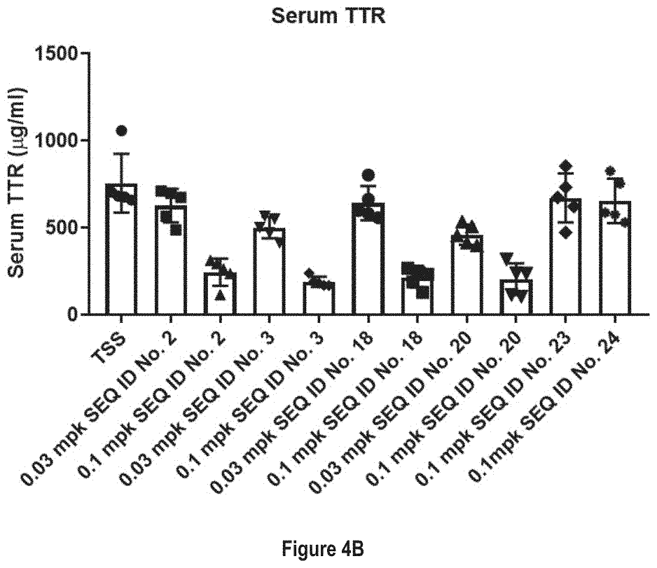

To determine editing efficiency of SEQ ID No. 18 and SEQ ID No. 20, an in vivo dose response experiment was performed. Messenger RNAs were produced and formulated with a 1:2 w/w ratio of chemically modified sgRNA:Cas9 mRNA as described in Example 1. The LNPs contained a guide RNA targeting TTR (G000502; SEQ ID NO: 4). CD-1 female mice (n=5 per group) were dosed i.v. at 0.03, 0.1, or 0.3 mpk. At 6 days post-dose, animals were sacrificed, blood and the liver were collected. Serum TTR and liver editing were measured. Table 15 and FIG. 4A show in vivo editing results. Table 15 and FIG. 4B show the serum TTR levels.

TABLE 15
Dose Response of Cas9 protein expression in vivo

| | Dose | % Editing | | Serum TTR (ug/ml) | | |
|---|---|---|---|---|---|---|
| mRNA | (mpk) | Mean | SD | Mean | SD | % TSS |
| TSS | | 0.2 | 0.2 | 755.9 | 169.6 | 100 |
| SEQ ID No. 2 | 0.03 | 9.9 | 3.8 | 627.3 | 96.4 | 83 |
| SEQ ID No. 2 | 0.1 | 48.9 | 7.9 | 244.8 | 78.0 | 32 |
| SEQ ID No. 3 | 0.03 | 21.7 | 3.5 | 500.8 | 61.8 | 66 |
| SEQ ID No. 3 | 0.1 | 53.6 | 8.3 | 190.4 | 29.4 | 25 |
| SEQ ID No. 18 | 0.03 | 12.2 | 1.9 | 641.3 | 98.5 | 85 |
| SEQ ID No. 18 | 0.1 | 48.1 | 7.6 | 214.5 | 55.9 | 28 |
| SEQ ID No. 20 | 0.03 | 18.4 | 5.2 | 460.3 | 58.2 | 61 |
| SEQ ID No. 20 | 0.1 | 46.1 | 8.3 | 205.1 | 90.8 | 27 |
| SEQ ID No. 23 | 0.1 | 1.9 | 2.3 | 671.2 | 140.6 | 89 |
| SEQ ID No. 24 | 0.1 | 4.3 | 1.6 | 654.5 | 127.5 | 87 |

### Example 6—Characterization of Expression from hSERPINA1 mRNA In Vitro

The level of protein expression from various codon optimized hSERPINA1 mRNAs in hepatocytes was tested by transfection. Capped and polyadenylated codon optimized SERPINA1 mRNAs were generated by in vitro transcription. Plasmid DNA template was linearized as described in Example 1. The IVT reaction to generate mRNA was performed by incubating at 37° C. for 4 hours in the following conditions: 50 ng/μL linearized plasmid; 5 mM each of GTP, ATP, CTP, and N1-methyl pseudo-UTP; 25 mM ARCA (Trilink); 7.5 U/μL T7 RNA polymerase (Roche); 1 U/μL Murine RNase inhibitor (Roche); 0.004 U/μL Inorganic E. coli pyrophosphatase (Roche); and 1× reaction buffer. TURBO DNase (ThermoFisher) was added to a final concentration of 0.01 U/μL, and the reaction was incubated for an additional 30 minutes to remove the DNA template.

Messenger RNAs were purified from enzyme and nucleotides using LiCl precipitation, ammonium acetate precipitation and sodium acetate precipitation. The transcript concentration was determined by measuring the light absorbance at 260 nm (Nanodrop), and the transcript was analyzed by capillary electrophoresis by Bioanlayzer (Agilent).

Primary mouse hepatocytes (PMH) and primary cyno hepatocytes (PCH) were cultured as described in Example 1. PMH and PCH were transfected with 200 ng of mRNA using 0.6 or 0.3 ul of MessengerMAX per well. Transfections were carried out according to manufacturer's protocol (ThermoFisher Scientific, Cat #LMRN003). Media was collected at post-treatment time points as indicated in Tables 16 and 17 to assay for hA1AT expression.

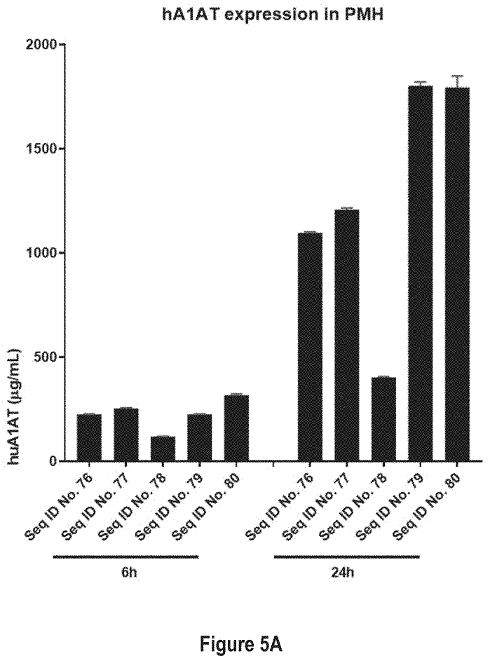

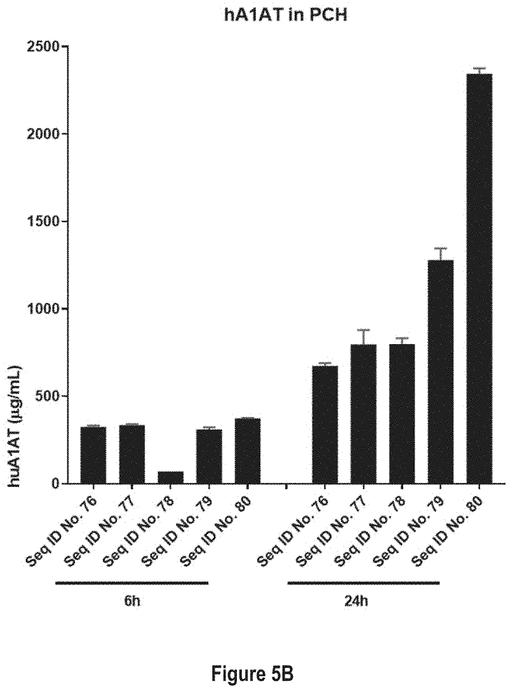

The hA1AT expression levels with codon optimized hSERPINA1 in this experiment are shown in FIG. 5A and Table 16 (PMH) and FIG. 5B and Table 17 (PCH). The transcripts of SEQ ID NOs: 76, 77, 78, 79, and 80 contain the SERPINA1 ORFs of SEQ ID NOs: 70, 69, 71, 72, and 73, respectively.

TABLE 16
hA1AT expression in Primary Mouse Hepatocytes

| | | | |
|---|---|---|---|
| | | Mean hA1AT | |
| Time Point | Sample | (ug/ml) | SD |

| | | | | |
|---|---|---|---|---|
| 6 | h | SEQ ID No: 76 | 226 | 2 |
| | | SEQ ID No: 77 | 255 | 2 |
| | | SEQ ID No: 78 | 120 | 1 |
| | | SEQ ID No: 79 | 225 | 2 |
| | | SEQ ID No: 80 | 318 | 7 |
| 24 | h | SEQ ID No: 76 | 1097 | 5 |
| | | SEQ ID No: 77 | 1209 | 8 |
| | | SEQ ID No: 78 | 403 | 4 |
| | | SEQ ID No: 79 | 1803 | 19 |
| | | SEQ ID No: 80 | 1795 | 55 |
| 48 | h | SEQ ID No: 76 | 120 | 6 |
| | | SEQ ID No: 77 | 166 | 0 |
| | | SEQ ID No: 78 | 81 | 1 |
| | | SEQ ID No: 79 | 210 | 3 |
| | | SEQ ID No: 80 | 284 | 3 |

TABLE 17
hA1AT expression in Primary Cyno Hepatocytes

| | | | |
|---|---|---|---|
| | | Mean hA1AT | |
| Time Point | Sample | (ug/ml) | STD |

| | | | | |
|---|---|---|---|---|
| 6 | h | media | 11 | 1 |
| | | SEQ ID No: 76 | 325 | 8 |
| | | SEQ ID No: 77 | 335 | 5 |
| | | SEQ ID No: 78 | 70 | 0 |
| | | SEQ ID No: 79 | 310 | 12 |
| | | SEQ ID No: 80 | 374 | 2 |
| 24 | h | media | 15 | 0 |
| | | SEQ ID No: 76 | 674 | 17 |
| | | SEQ ID No: 77 | 797 | 83 |
| | | SEQ ID No: 78 | 799 | 33 |
| | | SEQ ID No: 79 | 1280 | 66 |
| | | SEQ ID No: 80 | 2345 | 30 |

### Example 7—Characterization of Cas9 Expression in Primary Human Hepatocytes

Cas9 sequences using different codon schemes as described in Table 8 were designed to test for improved protein expression. Specifically, mRNAs having the sequences of SEQ ID NOs: 193 and 194, which contained ORFs according to SEQ ID NOs: 29 and 46, were tested in comparison to an mRNA having the sequence of SEQ ID NO: 3.

Translation efficiency was assessed in vitro by transfection of mRNA into primary human hepatocytes and measuring Cas9 protein expression levels by ELISA. Primary human liver hepatocytes (PHH) (Thermo Fisher) were cultured per standard protocols. In brief, the cells were thawed and resuspended in hepatocyte thawing medium (Thermo Fisher, Cat. CM7000) followed by centrifugation at 100 g for 10 minutes. The supernatant was discarded and the pelleted cells resuspended in hepatocyte plating medium plus supplement pack (Invitrogen, Cat. A1217601 and CM3000). Cells were counted and plated on Bio-coat collagen I coated 96-well plates (Thermo Fisher, Cat. 877272) at a density of 30,000-35,000 cells/well. Plated cells were allowed to settle and adhere for 4 to 6 hours in a tissue culture incubator at 37° C. and 5% CO₂ atmosphere. After incubation, cells were checked for monolayer formation. Cells were then washed with hepatocyte maintenance media/culture media with serum-free supplement pack (Invitrogen, Cat. A1217601 and CM4000) and then fresh hepatocyte maintenance media was added on to the cells.

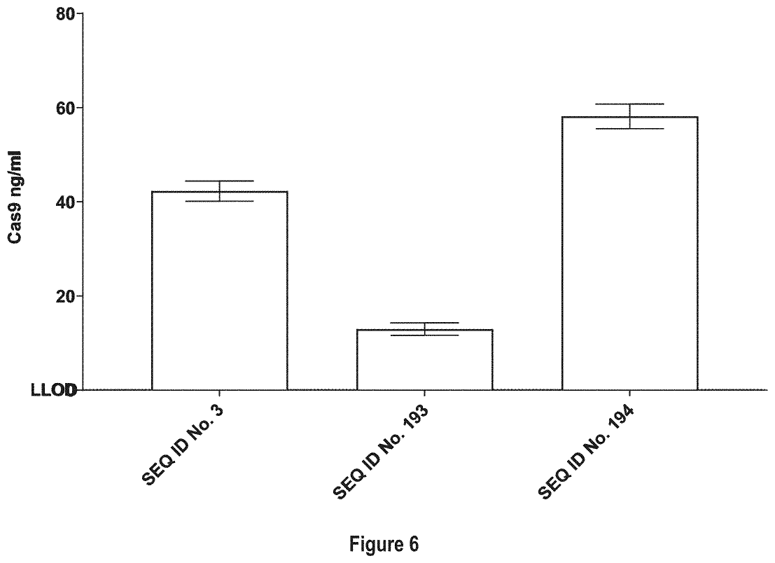

PHH cells were transfected with 150 ng of each Cas9 mRNA using Lipofectamine RNAiMAX (Fisher Scientific, Cat. 13778500) 24 hours after plating. Six hours post transfection, cells were lysed by freeze thaw and cleared by centrifugation. Cas9 protein expression was measured in these samples using the Meso Scale Discovery ELISA assay described in Example 1. Recombinant Cas9 protein was diluted in cleared PHH cell lysate to create a standard curve. Table 18 and FIG. 6 show the effects of the different codon schemes on Cas9 protein expression.

TABLE 18
In vitro expression of Cas9 protein
from ORFs with different codon sets

| | | Mean Cas9 | | |
|---|---|---|---|---|
| mRNA | ORF | protein | | |
| SEQ ID No. | SEQ ID No. | (ng/ml) | SD | N |
| 3 | | 42.3 | 2.1 | 3 |
| 193 | 29 | 13.0 | 1.3 | 3 |
| 194 | 46 | 58.1 | 2.6 | 3 |

### Example 8—Characterization of Cas9 Expression with Various UTRs

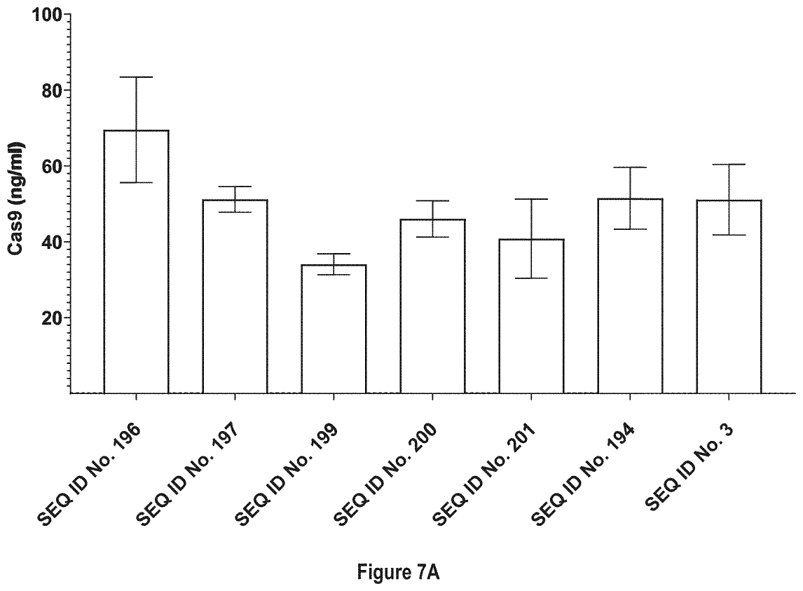

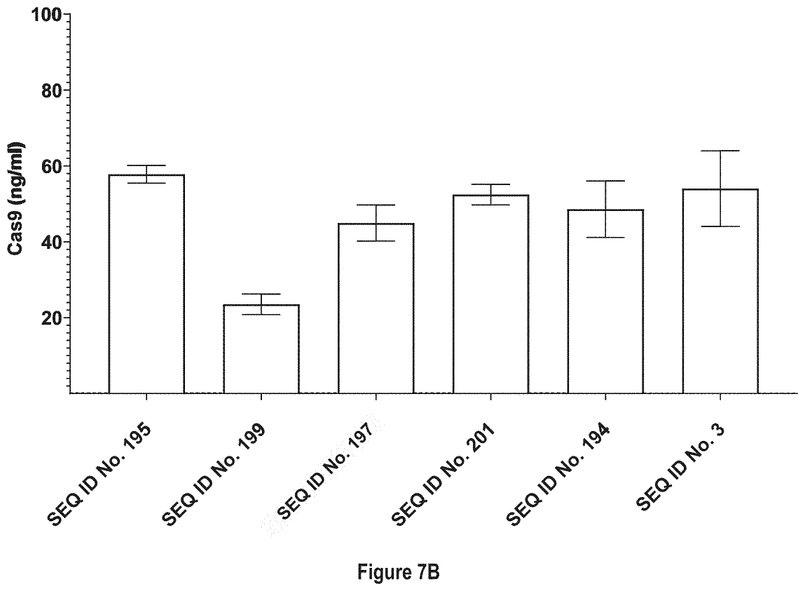

Select Cas9 ORFs were assayed for protein expression in combination with various 3′ UTRs, as described in Tables 19A-B. Translation efficiency was assessed in vitro by transfection of mRNA into primary human hepatocytes as in Example 7 and measuring Cas9 protein expression levels by ELISA as in Example 1. Tables 19A-B and FIGS. 7A-B show the Cas9 protein expression results.

TABLE 19A
In vitro expression of Cas9 protein with different 3′ UTRs

| | | | Mean Cas9 | | |
|---|---|---|---|---|---|
| mRNA | ORF | 3′UTR | protein | | |
| SEQ ID No. | SEQ ID No. | SEQ ID No. | (ng/ml) | SD | N |
| 196 | * | 202 | 69.5 | 13.9 | 3 |
| 197 | * | 203 | 51.2 | 3.4 | 3 |
| 199 | 46 | 204 | 34.1 | 2.7 | 3 |
| 200 | 46 | 202 | 46.0 | 4.8 | 3 |
| 201 | 46 | 203 | 40.8 | 10.4 | 3 |
| 194 | 46 | 184 | 51.5 | 8.1 | 3 |
| 3 | | 184 | 51.1 | 9.3 | 3 |

*The ORF of this mRNA is the Cas9 ORF of SEQ ID No. 3

TABLE 19B
In vitro expression of Cas9 protein with different 3′ UTRs

| | | | Mean Cas9 | | |
|---|---|---|---|---|---|
| mRNA | ORF | 3′UTR | protein | | |
| SEQ ID No. | SEQ ID No. | SEQ ID No. | (ng/ml) | SD | N |
| 195 | * | 204 | 57.8 | 2.3 | 3 |
| 199 | 46 | 204 | 23.5 | 2.7 | 3 |
| 197 | * | 203 | 44.9 | 4.8 | 3 |
| 201 | 46 | 203 | 52.4 | 2.7 | 3 |
| 194 | 46 | 184 | 48.6 | 7.4 | 3 |
| 3 | | 184 | 54.0 | 10.0 | 3 |

*The ORF of this mRNA is the Cas9 ORF of SEQ ID No. 3

Sequence Table
The following sequence table provides a listing of sequences disclosed herein. It is understood that if a DNA
sequence (comprising Ts) is referenced with respect to an RNA, then Ts should be replaced with Us (which may
be modified or unmodified depending on the context), and vice versa. *=PS linkage; ‘m’ = 2′-O-Me nucleotide.
For ORF descriptions, BP = I-pair depleted; GP = E-pair enriched; BS = I-single depleted; GS = E-single
enriched; GCU = subjected to steps of minimizing uridines, minimizing repeats, and maximizing GC content.
E-pairs, I-pairs, E-singles, and I-singles refer, respectively, to the codon pairs or codons of Tables 1-4.

| SEQ ID NO | Description | Sequence |
|---|---|---|
| 1 | Cas9 amino acid | MDKKYSIGLDIGTNSVGWAVITDEYKVPSKKFKVLGNTDRHSIKKNLIGALLFDSGETAEATRLKRTARRRYTRRKNRICYLQEIFS |
| | sequence | NEMAKVDDSFFHRLEESFLVEEDKKHERHPIFGNIVDEVAYHEKYPTIYHLRKKLVDSTDKADLRLIYLALAHMIKFRGHFLIEGDL |
| | | NPDNSDVDKLFIQLVQTYNQLFEENPINASGVDAKAILSARLSKSRRLENLIAQLPGEKKNGLFGNLIALSLGLTPNFKSNFDLAEDA |
| | | KLQLSKDTYDDDLDNLLAQIGDQYADLFLAAKNLSDAILLSDILRVNTEITKAPLSASMIKRYDEHHQDLTLLKALVRQQLPEKYKE |
| | | IFFDQSKNGYAGYIDGGASQEEFYKFIKPILEKMDGTEELLVKLNREDLLRKQRTFDNGSIPHQIHLGELHAILRRQEDFYPFLKDNRE |
| | | KIEKILTFRIPYYVGPLARGNSRFAWMTRKSEETITPWNFEEVVDKGASAQSFIERMTNFDKNLPNEKVLPKHSLLYEYFTVYNELTK |
| | | VKYVTEGMRKPAFLSGEQKKAIVDLLFKTNRKVTVKQLKEDYFKKIECFDSVEISGVEDRFNASLGTYHDLLKIIKDKDFLDNEENE |
| | | DILEDIVLTLTLFEDREMIEERLKTYAHLFDDKVMKQLKRRRYTGWGRLSRKLINGIRDKQSGKTILDFLKSDGFANRNFMQLIHDD |
| | | SLTFKEDIQKAQVSGQGDSLHEHIANLAGSPAIKKGILQTVKVVDELVKVMGRHKPENIVIEMARENQTTQKGQKNSRERMKRIEE |
| | | GIKELGSQILKEHPVENTQLQNEKLYLYYLQNGRDMYVDQELDINRLSDYDVDHIVPQSFLKDDSIDNKVLTRSDKNRGKSDNVPS |
| | | EEVVKKMKNYWRQLLNAKLITQRKFDNLTKAERGGLSELDKAGFIKRQLVETRQITKHVAQILDSRMNTKYDENDKLIREVKVITL |
| | | KSKLVSDFRKDFQFYKVREINNYHHAHDAYLNAVVGTALIKKYPKLESEFVYGDYKVYDVRKMIAKSEQEIGKATAKYFFYSNIM |
| | | NFFKTEITLANGEIRKRPLIETNGETGEIVWDKGRDFATVRKVLSMPQVNIVKKTEVQTGGFSKESILPKRNSDKLIARKKDWDPKK |
| | | YGGFDSPTVAYSVLVVAKVEKGKSKKLKSVKELLGITIMERSSFEKNPIDFLEAKGYKEVKKDLIIKLPKYSLFELENGRKRMLASA |
| | | GELQKGNELALPSKYVNFLYLASHYEKLKGSPEDNEQKQLFVEQHKHYLDEIIEQISEFSKRVILADANLDKVLSAYNKHRDKPIRE |
| | | QAENIIHLFTLTNLGAPAAFKYFDTTIDRKRYTSTKEVLDATLIHQSITGLYETRIDLSQLGGDGGGSPKKKRKV |
| 2 | Cas9 transcript | GGGUCCCGCAGUCGGCGUCCAGCGGCUCUGCUUGUUCGUGUGUGUGUCGUUGCAGGCCUUAUUCGGAUCCGCCACCAUGG |
| | with ORF having | ACAAGAAGUACAGCAUCGGACUGGACAUCGGAACAAACAGCGUCGGAUGGGCAGUCAUCACAGACGAAUACAAGGUCCCG |
| | low U content | AGCAAGAAGUUCAAGGUCCUGGGAAACACAGACAGACACAGCAUCAAGAAGAACCUGAUCGGAGCACUGCUGUUCGACAG |
| | | CGGAGAAACAGCAGAAGCAACAAGACUGAAGAGAACAGCAAGAAGAAGAUACACAAGAAGAAAGAACAGAAUCUGCUACC |
| | | UGCAGGAAAUCUUCAGCAACGAAAUGGCAAAGGUCGACGACAGCUUCUUCCACAGACUGGAAGAAAGCUUCCUGGUCGAA |
| | | GAAGACAAGAAGCACGAAAGACACCCGAUCUUCGGAAACAUCGUCGACGAAGUCGCAUACCACGAAAAGUACCCGACAAU |
| | | CUACCACCUGAGAAAGAAGCUGGUCGACAGCACAGACAAGGCAGACCUGAGACUGAUCUACCUGGCACUGGCACACAUGA |
| | | UCAAGUUCAGAGGACACUUCCUGAUCGAAGGAGACCUGAACCCGGACAACAGCGACGUCGACAAGCUGUUCAUCCAGCUG |
| | | GUCCAGACAUACAACCAGCUGUUCGAAGAAAACCCGAUCAACGCAAGCGGAGUCGACGCAAAGGCAAUCCUGAGCGCAAG |
| | | ACUGAGCAAGAGCAGAAGACUGGAAAACCUGAUCGCACAGCUGCCGGGAGAAAAGAAGAACGGACUGUUCGGAAACCUGA |
| | | UCGCACUGAGCCUGGGACUGACACCGAACUUCAAGAGCAACUUCGACCUGGCAGAAGACGCAAAGCUGCAGCUGAGCAAG |
| | | GACACAUACGACGACGACCUGGACAACCUGCUGGCACAGAUCGGAGACCAGUACGCAGACCUGUUCCUGGCAGCAAAGAA |
| | | CCUGAGCGACGCAAUCCUGCUGAGCGACAUCCUGAGAGUCAACACAGAAAUCACAAAGGCACCGCUGAGCGCAAGCAUGA |
| | | UCAAGAGAUACGACGAACACCACCAGGACCUGACACUGCUGAAGGCACUGGUCAGACAGCAGCUGCCGGAAAAGUACAAG |
| | | GAAAUCUUCUUCGACCAGAGCAAGAACGGAUACGCAGGAUACAUCGACGGAGGAGCAAGCCAGGAAGAAUUCUACAAGUU |
| | | CAUCAAGCCGAUCCUGGAAAAGAUGGACGGAACAGAAGAACUGCUGGUCAAGCUGAACAGAGAAGACCUGCUGAGAAAGC |
| | | AGAGAACAUUCGACAACGGAAGCAUCCCGCACCAGAUCCACCUGGGAGAACUGCACGCAAUCCUGAGAAGACAGGAAGAC |
| | | UUCUACCCGUUCCUGAAGGACAACAGAGAAAAGAUCGAAAAGAUCCUGACAUUCAGAAUCCCGUACUACGUCGGACCGCU |
| | | GGCAAGAGGAAACAGCAGAUUCGCAUGGAUGACAAGAAAGAGCGAAGAAACAAUCACACCGUGGAACUUCGAAGAAGUCG |
| | | UCGACAAGGGAGCAAGCGCACAGAGCUUCAUCGAAAGAAUGACAAACUUCGACAAGAACCUGCCGAACGAAAAGGUCCUG |
| | | CCGAAGCACAGCCUGCUGUACGAAUACUUCACAGUCUACAACGAACUGACAAAGGUCAAGUACGUCACAGAAGGAAUGAG |
| | | AAAGCCGGCAUUCCUGAGCGGAGAACAGAAGAAGGCAAUCGUCGACCUGCUGUUCAAGACAAACAGAAAGGUCACAGUCA |
| | | AGCAGCUGAAGGAAGACUACUUCAAGAAGAUCGAAUGCUUCGACAGCGUCGAAAUCAGCGGAGUCGAAGACAGAUUCAAC |
| | | GCAAGCCUGGGAACAUACCACGACCUGCUGAAGAUCAUCAAGGACAAGGACUUCCUGGACAACGAAGAAAACGAAGACAU |
| | | CCUGGAAGACAUCGUCCUGACACUGACACUGUUCGAAGACAGAGAAAUGAUCGAAGAAAGACUGAAGACAUACGCACACC |
| | | UGUUCGACGACAAGGUCAUGAAGCAGCUGAAGAGAAGAAGAUACACAGGAUGGGGAAGACUGAGCAGAAAGCUGAUCAA |
| | | CGGAAUCAGAGACAAGCAGAGCGGAAAGACAAUCCUGGACUUCCUGAAGAGCGACGGAUUCGCAAACAGAAACUUCAUGC |
| | | AGCUGAUCCACGACGACAGCCUGACAUUCAAGGAAGACAUCCAGAAGGCACAGGUCAGCGGACAGGGAGACAGCCUGCAC |
| | | GAACACAUCGCAAACCUGGCAGGAAGCCCGGCAAUCAAGAAGGGAAUCCUGCAGACAGUCAAGGUCGUCGACGAACUGGU |
| | | CAAGGUCAUGGGAAGACACAAGCCGGAAAACAUCGUCAUCGAAAUGGCAAGAGAAAACCAGACAACACAGAAGGGACAGA |
| | | AGAACAGCAGAGAAAGAAUGAAGAGAAUCGAAGAAGGAAUCAAGGAACUGGGAAGCCAGAUCCUGAAGGAACACCCGGU |
| | | CGAAAACACACAGCUGCAGAACGAAAAGCUGUACCUGUACUACCUGCAGAACGGAAGAGACAUGUACGUCGACCAGGAAC |
| | | UGGACAUCAACAGACUGAGCGACUACGACGUCGACCACAUCGUCCCGCAGAGCUUCCUGAAGGACGACAGCAUCGACAAC |
| | | AAGGUCCUGACAAGAAGCGACAAGAACAGAGGAAAGAGCGACAACGUCCCGAGCGAAGAAGUCGUCAAGAAGAUGAAGAA |
| | | CUACUGGAGACAGCUGCUGAACGCAAAGCUGAUCACACAGAGAAAGUUCGACAACCUGACAAAGGCAGAGAGAGGAGGAC |
| | | UGAGCGAACUGGACAAGGCAGGAUUCAUCAAGAGACAGCUGGUCGAAACAAGACAGAUCACAAAGCACGUCGCACAGAUC |
| | | CUGGACAGCAGAAUGAACACAAAGUACGACGAAAACGACAAGCUGAUCAGAGAAGUCAAGGUCAUCACACUGAAGAGCAA |
| | | GCUGGUCAGCGACUUCAGAAAGGACUUCCAGUUCUACAAGGUCAGAGAAAUCAACAACUACCACCACGCACACGACGCAU |
| | | ACCUGAACGCAGUCGUCGGAACAGCACUGAUCAAGAAGUACCCGAAGCUGGAAAGCGAAUUCGUCUACGGAGACUACAAG |
| | | GUCUACGACGUCAGAAAGAUGAUCGCAAAGAGCGAACAGGAAAUCGGAAAGGCAACAGCAAAGUACUUCUUCUACAGCAA |
| | | CAUCAUGAACUUCUUCAAGACAGAAAUCACACUGGCAAACGGAGAAAUCAGAAAGAGACCGCUGAUCGAAACAAACGGAG |
| | | AAACAGGAGAAAUCGUCUGGGACAAGGGAAGAGACUUCGCAACAGUCAGAAAGGUCCUGAGCAUGCCGCAGGUCAACAUC |
| | | GUCAAGAAGACAGAAGUCCAGACAGGAGGAUUCAGCAAGGAAAGCAUCCUGCCGAAGAGAAACAGCGACAAGCUGAUCGC |
| | | AAGAAAGAAGGACUGGGACCCGAAGAAGUACGGAGGAUUCGACAGCCCGACAGUCGCAUACAGCGUCCUGGUCGUCGCAA |
| | | AGGUCGAAAAGGGAAAGAGCAAGAAGCUGAAGAGCGUCAAGGAACUGCUGGGAAUCACAAUCAUGGAAAGAAGCAGCUU |
| | | CGAAAAGAACCCGAUCGACUUCCUGGAAGCAAAGGGAUACAAGGAAGUCAAGAAGGACCUGAUCAUCAAGCUGCCGAAGU |
| | | ACAGCCUGUUCGAACUGGAAAACGGAAGAAAGAGAAUGCUGGCAAGCGCAGGAGAACUGCAGAAGGGAAACGAACUGGCA |
| | | CUGCCGAGCAAGUACGUCAACUUCCUGUACCUGGCAAGCCACUACGAAAAGCUGAAGGGAAGCCCGGAAGACAACGAACA |
| | | GAAGCAGCUGUUCGUCGAACAGCACAAGCACUACCUGGACGAAAUCAUCGAACAGAUCAGCGAAUUCAGCAAGAGAGUCA |
| | | UCCUGGCAGACGCAAACCUGGACAAGGUCCUGAGCGCAUACAACAAGCACAGAGACAAGCCGAUCAGAGAACAGGCAGAA |
| | | AACAUCAUCCACCUGUUCACACUGACAAACCUGGGAGCACCGGCAGCAUUCAAGUACUUCGACACAACAAUCGACAGAAA |
| | | GAGAUACACAAGCACAAAGGAAGUCCUGGACGCAACACUGAUCCACCAGAGCAUCACAGGACUGUACGAAACAAGAAUCG |
| | | ACCUGAGCCAGCUGGGAGGAGACGGAGGAGGAAGCCCGAAGAAGAAGAGAAAGGUCUAGCUAGCCAUCACAUUUAAAAGC |
| | | AUCUCAGCCUACCAUGAGAAUAAGAGAAAGAAAAUGAAGAUCAAUAGCUUAUUCAUCUCUUUUUCUUUUUCGUUGGUGU |
| | | AAAGCCAACACCCUGUCUAAAAAACAUAAAUUUCUUUAAUCAUUUUGCCUCUUUUCUCUGUGCUUCAAUUAAUAAAAAAU |
| | | GGAAAGAACCUCGAGAAAAAAAAAAAAAAAAAAAAAAAAAAAAAAAAAAAAAAAAAAAAAAAAAAAAAAAAAAAAAAAA |
| | | AAAAAAAAAAAAAAAA |
| 3 | Cas9 transcript | GGGAAGCUCAGAAUAAACGCUCAACUUUGGCCGGAUCUGCCACCAUGGACAAGAAGUACUCCAUCGGCCUGGACAUCGGC |
| | with ORF having | ACCAACUCCGUGGGCUGGGCCGUGAUCACCGACGAGUACAAGGUGCCCUCCAAGAAGUUCAAGGUGCUGGGCAACACCGA |
| | low A content | CCGGCACUCCAUCAAGAAGAACCUGAUCGGCGCCCUGCUGUUCGACUCCGGCGAGACCGCCGAGGCCACCCGGCUGAAGCG |
| | | GACCGCCCGGCGGCGGUACACCCGGCGGAAGAACCGGAUCUGCUACCUGCAGGAGAUCUUCUCCAACGAGAUGGCCAAGG |
| | | UGGACGACUCCUUCUUCCACCGGCUGGAGGAGUCCUUCCUGGUGGAGGAGGACAAGAAGCACGAGCGGCACCCCAUCUUC |
| | | GGCAACAUCGUGGACGAGGUGGCCUACCACGAGAAGUACCCCACCAUCUACCACCUGCGGAAGAAGCUGGUGGACUCCAC |
| | | CGACAAGGCCGACCUGCGGCUGAUCUACCUGGCCCUGGCCCACAUGAUCAAGUUCCGGGGCCACUUCCUGAUCGAGGGCGA |
| | | CCUGAACCCCGACAACUCCGACGUGGACAAGCUGUUCAUCCAGCUGGUGCAGACCUACAACCAGCUGUUCGAGGAGAACCC |
| | | CAUCAACGCCUCCGGCGUGGACGCCAAGGCCAUCCUGUCCGCCCGGCUGUCCAAGUCCCGGCGGCUGGAGAACCUGAUCGC |
| | | CCAGCUGCCCGGCGAGAAGAAGAACGGCCUGUUCGGCAACCUGAUCGCCCUGUCCCUGGGCCUGACCCCCAACUUCAAGUC |
| | | CAACUUCGACCUGGCCGAGGACGCCAAGCUGCAGCUGUCCAAGGACACCUACGACGACGACCUGGACAACCUGCUGGCCCA |
| | | GAUCGGCGACCAGUACGCCGACCUGUUCCUGGCCGCCAAGAACCUGUCCGACGCCAUCCUGCUGUCCGACAUCCUGCGGGU |
| | | GAACACCGAGAUCACCAAGGCCCCCCUGUCCGCCUCCAUGAUCAAGCGGUACGACGAGCACCACCAGGACCUGACCCUGCU |
| | | GAAGGCCCUGGUGCGGCAGCAGCUGCCCGAGAAGUACAAGGAGAUCUUCUUCGACCAGUCCAAGAACGGCUACGCCGGCU |
| | | ACAUCGACGGCGGCGCCUCCCAGGAGGAGUUCUACAAGUUCAUCAAGCCCAUCCUGGAGAAGAUGGACGGCACCGAGGAG |
| | | CUGCUGGUGAAGCUGAACCGGGAGGACCUGCUGCGGAAGCAGCGGACCUUCGACAACGGCUCCAUCCCCCACCAGAUCCAC |
| | | CUGGGCGAGCUGCACGCCAUCCUGCGGCGGCAGGAGGACUUCUACCCCUUCCUGAAGGACAACCGGGAGAAGAUCGAGAA |
| | | GAUCCUGACCUUCCGGAUCCCCUACUACGUGGGCCCCCUGGCCCGGGGCAACUCCCGGUUCGCCUGGAUGACCCGGAAGUC |
| | | CGAGGAGACCAUCACCCCCUGGAACUUCGAGGAGGUGGUGGACAAGGGCGCCUCCGCCCAGUCCUUCAUCGAGCGGAUGA |
| | | CCAACUUCGACAAGAACCUGCCCAACGAGAAGGUGCUGCCCAAGCACUCCCUGCUGUACGAGUACUUCACCGUGUACAACG |
| | | AGCUGACCAAGGUGAAGUACGUGACCGAGGGCAUGCGGAAGCCCGCCUUCCUGUCCGGCGAGCAGAAGAAGGCCAUCGUG |
| | | GACCUGCUGUUCAAGACCAACCGGAAGGUGACCGUGAAGCAGCUGAAGGAGGACUACUUCAAGAAGAUCGAGUGCUUCGA |
| | | CUCCGUGGAGAUCUCCGGCGUGGAGGACCGGUUCAACGCCUCCCUGGGCACCUACCACGACCUGCUGAAGAUCAUCAAGG |
| | | ACAAGGACUUCCUGGACAACGAGGAGAACGAGGACAUCCUGGAGGACAUCGUGCUGACCCUGACCCUGUUCGAGGACCGG |
| | | GAGAUGAUCGAGGAGCGGCUGAAGACCUACGCCCACCUGUUCGACGACAAGGUGAUGAAGCAGCUGAAGCGGCGGCGGUA |
| | | CACCGGCUGGGGCCGGCUGUCCCGGAAGCUGAUCAACGGCAUCCGGGACAAGCAGUCCGGCAAGACCAUCCUGGACUUCCU |
| | | GAAGUCCGACGGCUUCGCCAACCGGAACUUCAUGCAGCUGAUCCACGACGACUCCCUGACCUUCAAGGAGGACAUCCAGA |
| | | AGGCCCAGGUGUCCGGCCAGGGCGACUCCCUGCACGAGCACAUCGCCAACCUGGCCGGCUCCCCCGCCAUCAAGAAGGGCA |
| | | UCCUGCAGACCGUGAAGGUGGUGGACGAGCUGGUGAAGGUGAUGGGCCGGCACAAGCCCGAGAACAUCGUGAUCGAGAUG |
| | | GCCCGGGAGAACCAGACCACCCAGAAGGGCCAGAAGAACUCCCGGGAGCGGAUGAAGCGGAUCGAGGAGGGCAUCAAGGA |
| | | GCUGGGCUCCCAGAUCCUGAAGGAGCACCCCGUGGAGAACACCCAGCUGCAGAACGAGAAGCUGUACCUGUACUACCUGC |
| | | AGAACGGCCGGGACAUGUACGUGGACCAGGAGCUGGACAUCAACCGGCUGUCCGACUACGACGUGGACCACAUCGUGCCC |
| | | CAGUCCUUCCUGAAGGACGACUCCAUCGACAACAAGGUGCUGACCCGGUCCGACAAGAACCGGGGCAAGUCCGACAACGU |
| | | GCCCUCCGAGGAGGUGGUGAAGAAGAUGAAGAACUACUGGCGGCAGCUGCUGAACGCCAAGCUGAUCACCCAGCGGAAGU |
| | | UCGACAACCUGACCAAGGCCGAGCGGGGCGGCCUGUCCGAGCUGGACAAGGCCGGCUUCAUCAAGCGGCAGCUGGUGGAG |
| | | ACCCGGCAGAUCACCAAGCACGUGGCCCAGAUCCUGGACUCCCGGAUGAACACCAAGUACGACGAGAACGACAAGCUGAU |
| | | CCGGGAGGUGAAGGUGAUCACCCUGAAGUCCAAGCUGGUGUCCGACUUCCGGAAGGACUUCCAGUUCUACAAGGUGCGGG |
| | | AGAUCAACAACUACCACCACGCCCACGACGCCUACCUGAACGCCGUGGUGGGCACCGCCCUGAUCAAGAAGUACCCCAAGC |
| | | UGGAGUCCGAGUUCGUGUACGGCGACUACAAGGUGUACGACGUGCGGAAGAUGAUCGCCAAGUCCGAGCAGGAGAUCGGC |
| | | AAGGCCACCGCCAAGUACUUCUUCUACUCCAACAUCAUGAACUUCUUCAAGACCGAGAUCACCCUGGCCAACGGCGAGAUC |
| | | CGGAAGCGGCCCCUGAUCGAGACCAACGGCGAGACCGGCGAGAUCGUGUGGGACAAGGGCCGGGACUUCGCCACCGUGCG |
| | | GAAGGUGCUGUCCAUGCCCCAGGUGAACAUCGUGAAGAAGACCGAGGUGCAGACCGGCGGCUUCUCCAAGGAGUCCAUCC |
| | | UGCCCAAGCGGAACUCCGACAAGCUGAUCGCCCGGAAGAAGGACUGGGACCCCAAGAAGUACGGCGGCUUCGACUCCCCCA |
| | | CCGUGGCCUACUCCGUGCUGGUGGUGGCCAAGGUGGAGAAGGGCAAGUCCAAGAAGCUGAAGUCCGUGAAGGAGCUGCUG |
| | | GGCAUCACCAUCAUGGAGCGGUCCUCCUUCGAGAAGAACCCCAUCGACUUCCUGGAGGCCAAGGGCUACAAGGAGGUGAA |
| | | GAAGGACCUGAUCAUCAAGCUGCCCAAGUACUCCCUGUUCGAGCUGGAGAACGGCCGGAAGCGGAUGCUGGCCUCCGCCG |
| | | GCGAGCUGCAGAAGGGCAACGAGCUGGCCCUGCCCUCCAAGUACGUGAACUUCCUGUACCUGGCCUCCCACUACGAGAAGC |
| | | UGAAGGGCUCCCCCGAGGACAACGAGCAGAAGCAGCUGUUCGUGGAGCAGCACAAGCACUACCUGGACGAGAUCAUCGAG |
| | | CAGAUCUCCGAGUUCUCCAAGCGGGUGAUCCUGGCCGACGCCAACCUGGACAAGGUGCUGUCCGCCUACAACAAGCACCGG |
| | | GACAAGCCCAUCCGGGAGCAGGCCGAGAACAUCAUCCACCUGUUCACCCUGACCAACCUGGGCGCCCCCGCCGCCUUCAAG |
| | | UACUUCGACACCACCAUCGACCGGAAGCGGUACACCUCCACCAAGGAGGUGCUGGACGCCACCCUGAUCCACCAGUCCAUC |
| | | ACCGGCCUGUACGAGACCCGGAUCGACCUGUCCCAGCUGGGCGGCGACGGCGGCGGCUCCCCCAAGAAGAAGCGGAAGGU |
| | | GUGACUAGCACCAGCCUCAAGAACACCCGAAUGGAGUCUCUAAGCUACAUAAUACCAACUUACACUUUACAAAAUGUUGU |
| | | CCCCCAAAAUGUAGCCAUUCGUAUCUGCUCCUAAUAAAAAGAAAGUUUCUUCACAUUCUCUCGAGAAAAAAAAAAAAAAA |
| | | AAAAAAAAAAAAAAAAAAAAAAAAAAAAAAAAAAAAAAAAAAAAAAAAAAAAAAAAAAAAAAAAA |
| 4 | Guide RNA | mA*mC*mA*CAAAUACCAGUCCAGCGGUUUUAGAmGmCmUmAmGmAmAmAmUmAmGmCAAGUUAAAAUAAGGCUAGUCC |
| | G000502 | GUUAUCAmAmCmUmUmGmAmAmAmAmAmGmUmGmGmCmAmCmCmGmAmGmUmCmGmGmUmGmCmU*mU*mU*mU |
| 5 | E-single | AUGGACAAGAAGUACAGCAUCGGCCUGGACAUCGGCACGAACAGCGUGGGCUGGGCCGUGAUCACGGACGAGUACAAGGU |
| | enriched Cas9 | GCCCAGCAAGAAGUUCAAGGUGCUGGGCAACACGGACCGGCACAGCAUCAAGAAGAACCUGAUCGGCGCCCUGCUGUUCG |
| | ORF | ACAGCGGCGAGACGGCCGAGGCCACGCGGCUGAAGCGGACGGCCCGGCGGCGGUACACGCGGCGGAAGAACCGGAUCUGC |
| | | UACCUGCAGGAGAUCUUCAGCAACGAGAUGGCCAAGGUGGACGACAGCUUCUUCCACCGGCUGGAGGAGAGCUUCCUGGU |
| | | GGAGGAGGACAAGAAGCACGAGCGGCACCCCAUCUUCGGCAACAUCGUGGACGAGGUGGCCUACCACGAGAAGUACCCCA |
| | | CGAUCUACCACCUGCGGAAGAAGCUGGUGGACAGCACGGACAAGGCCGACCUGCGGCUGAUCUACCUGGCCCUGGCCCACA |
| | | UGAUCAAGUUCCGGGGCCACUUCCUGAUCGAGGGCGACCUGAACCCCGACAACAGCGACGUGGACAAGCUGUUCAUCCAG |
| | | CUGGUGCAGACGUACAACCAGCUGUUCGAGGAGAACCCCAUCAACGCCAGCGGCGUGGACGCCAAGGCCAUCCUGAGCGCC |
| | | CGGCUGAGCAAGAGCCGGCGGCUGGAGAACCUGAUCGCCCAGCUGCCCGGCGAGAAGAAGAACGGCCUGUUCGGCAACCU |
| | | GAUCGCCCUGAGCCUGGGCCUGACGCCCAACUUCAAGAGCAACUUCGACCUGGCCGAGGACGCCAAGCUGCAGCUGAGCAA |
| | | GGACACGUACGACGACGACCUGGACAACCUGCUGGCCCAGAUCGGCGACCAGUACGCCGACCUGUUCCUGGCCGCCAAGAA |
| | | CCUGAGCGACGCCAUCCUGCUGAGCGACAUCCUGCGGGUGAACACGGAGAUCACGAAGGCCCCCCUGAGCGCCAGCAUGAU |
| | | CAAGCGGUACGACGAGCACCACCAGGACCUGACGCUGCUGAAGGCCCUGGUGCGGCAGCAGCUGCCCGAGAAGUACAAGG |
| | | AGAUCUUCUUCGACCAGAGCAAGAACGGCUACGCCGGCUACAUCGACGGCGGCGCCAGCCAGGAGGAGUUCUACAAGUUC |
| | | AUCAAGCCCAUCCUGGAGAAGAUGGACGGCACGGAGGAGCUGCUGGUGAAGCUGAACCGGGAGGACCUGCUGCGGAAGCA |
| | | GCGGACGUUCGACAACGGCAGCAUCCCCCACCAGAUCCACCUGGGCGAGCUGCACGCCAUCCUGCGGCGGCAGGAGGACUU |
| | | CUACCCCUUCCUGAAGGACAACCGGGAGAAGAUCGAGAAGAUCCUGACGUUCCGGAUCCCCUACUACGUGGGCCCCCUGGC |
| | | CCGGGGCAACAGCCGGUUCGCCUGGAUGACGCGGAAGAGCGAGGAGACGAUCACGCCCUGGAACUUCGAGGAGGUGGUGG |
| | | ACAAGGGCGCCAGCGCCCAGAGCUUCAUCGAGCGGAUGACGAACUUCGACAAGAACCUGCCCAACGAGAAGGUGCUGCCC |
| | | AAGCACAGCCUGCUGUACGAGUACUUCACGGUGUACAACGAGCUGACGAAGGUGAAGUACGUGACGGAGGGCAUGCGGAA |
| | | GCCCGCCUUCCUGAGCGGCGAGCAGAAGAAGGCCAUCGUGGACCUGCUGUUCAAGACGAACCGGAAGGUGACGGUGAAGC |
| | | AGCUGAAGGAGGACUACUUCAAGAAGAUCGAGUGCUUCGACAGCGUGGAGAUCAGCGGCGUGGAGGACCGGUUCAACGCC |
| | | AGCCUGGGCACGUACCACGACCUGCUGAAGAUCAUCAAGGACAAGGACUUCCUGGACAACGAGGAGAACGAGGACAUCCU |
| | | GGAGGACAUCGUGCUGACGCUGACGCUGUUCGAGGACCGGGAGAUGAUCGAGGAGCGGCUGAAGACGUACGCCCACCUGU |
| | | UCGACGACAAGGUGAUGAAGCAGCUGAAGCGGCGGCGGUACACGGGCUGGGGCCGGCUGAGCCGGAAGCUGAUCAACGGC |
| | | AUCCGGGACAAGCAGAGCGGCAAGACGAUCCUGGACUUCCUGAAGAGCGACGGCUUCGCCAACCGGAACUUCAUGCAGCU |
| | | GAUCCACGACGACAGCCUGACGUUCAAGGAGGACAUCCAGAAGGCCCAGGUGAGCGGCCAGGGCGACAGCCUGCACGAGC |
| | | ACAUCGCCAACCUGGCCGGCAGCCCCGCCAUCAAGAAGGGCAUCCUGCAGACGGUGAAGGUGGUGGACGAGCUGGUGAAG |
| | | GUGAUGGGCCGGCACAAGCCCGAGAACAUCGUGAUCGAGAUGGCCCGGGAGAACCAGACGACGCAGAAGGGCCAGAAGAA |
| | | CAGCCGGGAGCGGAUGAAGCGGAUCGAGGAGGGCAUCAAGGAGCUGGGCAGCCAGAUCCUGAAGGAGCACCCCGUGGAGA |
| | | ACACGCAGCUGCAGAACGAGAAGCUGUACCUGUACUACCUGCAGAACGGCCGGGACAUGUACGUGGACCAGGAGCUGGAC |
| | | AUCAACCGGCUGAGCGACUACGACGUGGACCACAUCGUGCCCCAGAGCUUCCUGAAGGACGACAGCAUCGACAACAAGGU |
| | | GCUGACGCGGAGCGACAAGAACCGGGGCAAGAGCGACAACGUGCCCAGCGAGGAGGUGGUGAAGAAGAUGAAGAACUACU |
| | | GGCGGCAGCUGCUGAACGCCAAGCUGAUCACGCAGCGGAAGUUCGACAACCUGACGAAGGCCGAGCGGGGCGGCCUGAGC |
| | | GAGCUGGACAAGGCCGGCUUCAUCAAGCGGCAGCUGGUGGAGACGCGGCAGAUCACGAAGCACGUGGCCCAGAUCCUGGA |
| | | CAGCCGGAUGAACACGAAGUACGACGAGAACGACAAGCUGAUCCGGGAGGUGAAGGUGAUCACGCUGAAGAGCAAGCUGG |
| | | UGAGCGACUUCCGGAAGGACUUCCAGUUCUACAAGGUGCGGGAGAUCAACAACUACCACCACGCCCACGACGCCUACCUG |
| | | AACGCCGUGGUGGGCACGGCCCUGAUCAAGAAGUACCCCAAGCUGGAGAGCGAGUUCGUGUACGGCGACUACAAGGUGUA |
| | | CGACGUGCGGAAGAUGAUCGCCAAGAGCGAGCAGGAGAUCGGCAAGGCCACGGCCAAGUACUUCUUCUACAGCAACAUCA |
| | | UGAACUUCUUCAAGACGGAGAUCACGCUGGCCAACGGCGAGAUCCGGAAGCGGCCCCUGAUCGAGACGAACGGCGAGACG |
| | | GGCGAGAUCGUGUGGGACAAGGGCCGGGACUUCGCCACGGUGCGGAAGGUGCUGAGCAUGCCCCAGGUGAACAUCGUGAA |
| | | GAAGACGGAGGUGCAGACGGGCGGCUUCAGCAAGGAGAGCAUCCUGCCCAAGCGGAACAGCGACAAGCUGAUCGCCCGGA |
| | | AGAAGGACUGGGACCCCAAGAAGUACGGCGGCUUCGACAGCCCCACGGUGGCCUACAGCGUGCUGGUGGUGGCCAAGGUG |
| | | GAGAAGGGCAAGAGCAAGAAGCUGAAGAGCGUGAAGGAGCUGCUGGGCAUCACGAUCAUGGAGCGGAGCAGCUUCGAGA |
| | | AGAACCCCAUCGACUUCCUGGAGGCCAAGGGCUACAAGGAGGUGAAGAAGGACCUGAUCAUCAAGCUGCCCAAGUACAGC |
| | | CUGUUCGAGCUGGAGAACGGCCGGAAGCGGAUGCUGGCCAGCGCCGGCGAGCUGCAGAAGGGCAACGAGCUGGCCCUGCC |
| | | CAGCAAGUACGUGAACUUCCUGUACCUGGCCAGCCACUACGAGAAGCUGAAGGGCAGCCCCGAGGACAACGAGCAGAAGC |
| | | AGCUGUUCGUGGAGCAGCACAAGCACUACCUGGACGAGAUCAUCGAGCAGAUCAGCGAGUUCAGCAAGCGGGUGAUCCUG |
| | | GCCGACGCCAACCUGGACAAGGUGCUGAGCGCCUACAACAAGCACCGGGACAAGCCCAUCCGGGAGCAGGCCGAGAACAUC |
| | | AUCCACCUGUUCACGCUGACGAACCUGGGCGCCCCCGCCGCCUUCAAGUACUUCGACACGACGAUCGACCGGAAGCGGUAC |
| | | ACGAGCACGAAGGAGGUGCUGGACGCCACGCUGAUCCACCAGAGCAUCACGGGCCUGUACGAGACGCGGAUCGACCUGAG |
| | | CCAGCUGGGCGGCGACGGCGGCGGCAGCCCCAAGAAGAAGCGGAAGGUGUAG |
| 6 | E-pair enriched, | AUGGACAAGAAGUACAGCAUCGGCCUGGACAUCGGCACGAACAGCGUUGGCUGGGCUGUGAUCACGGACGAGUACAAGGU |
| | I-pair depleted | UCCCAGCAAGAAGUUCAAGGUGCUGGGCAACACGGACCGGCACAGCAUCAAGAAGAAUCUGAUCGGUGCACUGCUGUUCG |
| | Cas9 ORF | ACAGCGGUGAGACGGCCGAAGCCACGCGGCUGAAGCGGACGGCCCGGCGGCGGUACACGCGGCGGAAGAACCGGAUCUGC |
| | | UACCUGCAGGAGAUCUUCAGCAACGAGAUGGCCAAGGUGGACGACAGCUUCUUCCACCGGCUGGAGGAGAGCUUCCUGGU |
| | | GGAGGAGGACAAGAAGCACGAGCGGCACCCCAUCUUCGGCAACAUCGUGGACGAAGUGGCCUACCACGAGAAGUACCCCA |
| | | CGAUCUACCACCUGCGGAAGAAGCUGGUGGACUCGACGGACAAGGCCGACCUGCGGCUGAUCUACCUGGCACUGGCCCAC |
| | | AUGAUCAAGUUCCGGGGCCACUUCCUGAUCGAGGGCGACCUGAACCCUGACAACAGCGACGUGGACAAGCUGUUCAUCCA |
| | | GCUGGUGCAGACCUACAACCAGCUGUUCGAGGAGAACCCCAUCAACGCCAGCGGCGUGGACGCCAAGGCCAUCCUCAGCGC |
| | | CCGGCUCAGCAAGAGCCGGCGGCUGGAGAAUCUGAUCGCCCAGCUUCCCGGUGAGAAGAAGAAUGGCCUGUUCGGCAAUC |
| | | UGAUCGCACUCAGCCUGGGCCUGACUCCCAACUUCAAGAGCAACUUCGACCUGGCCGAGGACGCCAAGCUGCAGCUCAGCA |
| | | AGGACACCUACGACGACGACCUGGACAAUCUGCUGGCCCAGAUCGGCGACCAGUACGCCGACCUGUUCCUGGCUGCCAAGA |
| | | AUCUCAGCGACGCCAUCCUGCUCAGCGACAUCCUGCGGGUGAACACAGAGAUCACGAAGGCCCCCCUCAGCGCCAGCAUGA |
| | | UCAAGCGGUACGACGAGCACCACCAGGACCUGACGCUGCUGAAGGCACUGGUGCGGCAGCAGCUUCCAGAGAAGUACAAG |
| | | GAGAUCUUCUUCGACCAGAGCAAGAAUGGCUACGCCGGCUACAUCGACGGUGGUGCCAGCCAGGAGGAGUUCUACAAGUU |
| | | CAUCAAGCCCAUCCUGGAGAAGAUGGACGGCACAGAGGAGCUGCUGGUGAAGCUGAACAGGGAGGACCUGCUGCGGAAGC |
| | | AGCGGACGUUCGACAAUGGCAGCAUCCCCCACCAGAUCCACCUGGGUGAGCUGCACGCCAUCCUGCGGCGGCAGGAGGACU |
| | | UCUACCCCUUCCUGAAGGACAACAGGGAGAAGAUCGAGAAGAUCCUGACGUUCCGGAUCCCCUACUACGUUGGCCCCCUG |
| | | GCCCGGGGCAACAGCCGGUUCGCCUGGAUGACGCGGAAGAGCGAGGAGACGAUCACUCCCUGGAACUUCGAGGAAGUGGU |
| | | GGACAAGGGUGCCAGCGCCCAGAGCUUCAUCGAGCGGAUGACGAACUUCGACAAGAAUCUUCCCAACGAGAAGGUGCUUC |
| | | CCAAGCACAGCCUGCUGUACGAGUACUUCACGGUGUACAACGAGCUGACGAAGGUGAAGUACGUGACAGAGGGCAUGCGG |
| | | AAGCCCGCCUUCCUCAGCGGUGAGCAGAAGAAGGCCAUCGUGGACCUGCUGUUCAAGACGAACCGGAAGGUGACGGUGAA |
| | | GCAGCUGAAGGAGGACUACUUCAAGAAGAUCGAGUGCUUCGACAGCGUGGAGAUCAGCGGCGUGGAGGACCGGUUCAACG |
| | | CCAGCCUGGGCACCUACCACGACCUGCUGAAGAUCAUCAAGGACAAGGACUUCCUGGACAACGAGGAGAACGAGGACAUC |
| | | CUGGAGGACAUCGUGCUGACGCUGACGCUGUUCGAGGACAGGGAGAUGAUCGAGGAGCGGCUGAAGACCUACGCCCACCU |
| | | GUUCGACGACAAGGUGAUGAAGCAGCUGAAGCGGCGGCGGUACACGGGCUGGGGCCGGCUCAGCCGGAAGCUGAUCAAUG |
| | | GCAUCCGAGACAAGCAGAGCGGCAAGACGAUCCUGGACUUCCUGAAGAGCGACGGCUUCGCCAACCGGAACUUCAUGCAG |
| | | CUGAUCCACGACGACAGCCUGACGUUCAAGGAGGACAUCCAGAAGGCCCAGGUCAGCGGCCAGGGCGACAGCCUGCACGA |
| | | GCACAUCGCCAAUCUGGCCGGCAGCCCCGCCAUCAAGAAGGGCAUCCUGCAGACGGUGAAGGUGGUGGACGAGCUGGUGA |
| | | AGGUGAUGGGCCGGCACAAGCCAGAGAACAUCGUGAUCGAGAUGGCCAGGGAGAACCAGACGACUCAGAAGGGCCAGAAG |
| | | AACAGCAGGGAGCGGAUGAAGCGGAUCGAGGAGGGCAUCAAGGAGCUGGGCAGCCAGAUCCUGAAGGAGCACCCCGUGGA |
| | | GAACACUCAGCUGCAGAACGAGAAGCUGUACCUGUACUACCUGCAGAAUGGCCGAGACAUGUACGUGGACCAGGAGCUGG |
| | | ACAUCAACCGGCUCAGCGACUACGACGUGGACCACAUCGUUCCCCAGAGCUUCCUGAAGGACGACAGCAUCGACAACAAG |
| | | GUGCUGACGCGGAGCGACAAGAACCGGGGCAAGAGCGACAACGUUCCCAGCGAGGAAGUGGUGAAGAAGAUGAAGAACUA |
| | | CUGGCGGCAGCUGCUGAACGCCAAGCUGAUCACUCAGCGGAAGUUCGACAAUCUGACGAAGGCCGAGCGGGGUGGCCUCA |
| | | GCGAGCUGGACAAGGCCGGCUUCAUCAAGCGGCAGCUGGUGGAGACGCGGCAGAUCACGAAGCACGUGGCCCAGAUCCUG |
| | | GACAGCCGGAUGAACACGAAGUACGACGAGAACGACAAGCUGAUCAGGGAAGUGAAGGUGAUCACGCUGAAGAGCAAGCU |
| | | GGUCAGCGACUUCCGGAAGGACUUCCAGUUCUACAAGGUGAGGGAGAUCAACAACUACCACCACGCCCACGACGCCUACC |
| | | UGAACGCUGUGGUUGGCACGGCACUGAUCAAGAAGUACCCCAAGCUGGAGAGCGAGUUCGUGUACGGCGACUACAAGGUG |
| | | UACGACGUGCGGAAGAUGAUCGCCAAGAGCGAGCAGGAGAUCGGCAAGGCCACGGCCAAGUACUUCUUCUACAGCAACAU |
| | | CAUGAACUUCUUCAAGACAGAGAUCACGCUGGCCAAUGGUGAGAUCCGGAAGCGGCCCCUGAUCGAGACGAAUGGUGAGA |
| | | CGGGUGAGAUCGUGUGGGACAAGGGCCGAGACUUCGCCACGGUGCGGAAGGUGCUCAGCAUGCCCCAGGUGAACAUCGUG |
| | | AAGAAGACAGAAGUGCAGACGGGUGGCUUCAGCAAGGAGAGCAUCCUUCCCAAGCGGAACAGCGACAAGCUGAUCGCCCG |
| | | GAAGAAGGACUGGGACCCCAAGAAGUACGGUGGCUUCGACAGCCCCACGGUGGCCUACAGCGUGCUGGUGGUGGCCAAGG |
| | | UGGAGAAGGGCAAGAGCAAGAAGCUGAAGAGCGUGAAGGAGCUGCUGGGCAUCACGAUCAUGGAGCGGAGCAGCUUCGA |
| | | GAAGAACCCCAUCGACUUCCUGGAAGCCAAGGGCUACAAGGAAGUGAAGAAGGACCUGAUCAUCAAGCUUCCCAAGUACA |
| | | GCCUGUUCGAGCUGGAGAAUGGCCGGAAGCGGAUGCUGGCCAGCGCCGGUGAGCUGCAGAAGGGCAACGAGCUGGCACUU |
| | | CCCAGCAAGUACGUGAACUUCCUGUACCUGGCCAGCCACUACGAGAAGCUGAAGGGCAGCCCAGAGGACAACGAGCAGAA |
| | | GCAGCUGUUCGUGGAGCAGCACAAGCACUACCUGGACGAGAUCAUCGAGCAGAUCAGCGAGUUCAGCAAGCGGGUGAUCC |
| | | UGGCCGACGCCAAUCUGGACAAGGUGCUCAGCGCCUACAACAAGCACCGAGACAAGCCCAUCAGGGAGCAGGCCGAGAAC |
| | | AUCAUCCACCUGUUCACGCUGACGAAUCUGGGUGCCCCCGCUGCCUUCAAGUACUUCGACACGACGAUCGACCGGAAGCGG |
| | | UACACGUCGACGAAGGAAGUGCUGGACGCCACGCUGAUCCACCAGAGCAUCACGGGCCUGUACGAGACGCGGAUCGACCU |
| | | CAGCCAGCUGGGUGGCGACGGUGGUGGCAGCCCCAAGAAGAAGCGGAAGGUGUAG |
| 7 | E-pair & E- | AUGGACAAGAAGUACAGCAUCGGCCUCGACAUCGGCACCAACAGCGUUGGCUGGGCUGUGAUCACCGACGAGUACAAGGU |
| | single enriched, | UCCCUCAAAGAAGUUCAAGGUCCUCGGCAACACCGACCGCCACAGCAUCAAGAAGAAUCUCAUCGGUGCACUGCUCUUCG |
| | I-pair & I-single | ACAGCGGUGAGACCGCGGAAGCCACCCGCCUCAAGCGGACCGCCCGCCGCCGCUACACCCGCCGCAAGAACCGCAUCUGCU |
| | depleted Cas9 | ACCUCCAGGAGAUCUUCAGCAACGAGAUGGCCAAGGUCGACGACAGCUUCUUCCACCGCCUCGAGGAGAGCUUCCUGGUC |
| | ORF | GAGGAGGACAAGAAGCACGAGCGCCACCCCAUCUUCGGCAACAUCGUCGACGAAGUCGCCUACCACGAGAAGUACCCCACC |
| | | AUCUACCACCUGCGGAAGAAGCUCGUCGACUCGACUGACAAGGCCGACCUGCGGCUCAUCUACCUCGCACUGGCCCACAUG |
| | | AUAAAGUUCCGCGGCCACUUCCUGAUCGAGGGCGACCUCAACCCUGACAACAGCGACGUCGACAAGCUCUUCAUCCAGCUC |
| | | GUCCAGACCUACAACCAGCUCUUCGAGGAGAACCCCAUCAACGCCAGCGGCGUCGACGCCAAGGCCAUCCUCAGCGCCCGC |
| | | CUCAGCAAGAGCCGCCGCCUCGAGAAUCUCAUCGCCCAGCUUCCAGGUGAGAAGAAGAAUGGGCUCUUCGGCAAUCUCAU |
| | | CGCACUCAGCCUCGGCCUCACUCCCAACUUCAAGAGCAACUUCGACCUCGCGGAGGACGCCAAGCUCCAGCUCAGCAAGGA |
| | | CACCUACGACGACGACCUCGACAAUCUCCUCGCCCAGAUCGGCGACCAGUACGCCGACCUCUUCCUGGCUGCCAAGAAUCU |
| | | CAGCGACGCCAUCCUCCUCAGCGACAUCCUGCGGGUCAACACAGAGAUCACCAAGGCCCCCCUCAGCGCCAGCAUGAUAAA |
| | | GCGCUACGACGAGCACCACCAGGACCUCACCCUCCUCAAGGCACUGGUCCGCCAGCAGCUUCCAGAGAAGUACAAGGAGAU |
| | | CUUCUUCGACCAGAGCAAGAAUGGGUACGCCGGGUACAUCGACGGUGGUGCCAGCCAGGAGGAGUUCUACAAGUUCAUCA |
| | | AGCCCAUCCUCGAGAAGAUGGACGGCACAGAGGAGCUGCUCGUCAAGCUCAACAGGGAGGACCUCCUGCGGAAGCAGCGG |
| | | ACCUUCGACAAUGGGAGCAUCCCCCACCAGAUCCACCUCGGUGAGCUGCACGCCAUCCUGCGGCGCCAGGAGGACUUCUAC |
| | | CCCUUCCUGAAGGACAACAGGGAGAAGAUCGAGAAGAUCCUCACCUUCCGCAUCCCCUACUACGUUGGCCCCCUCGCCCGC |
| | | GGCAACAGCCGCUUCGCCUGGAUGACCCGCAAGAGCGAGGAGACCAUCACUCCCUGGAACUUCGAGGAAGUCGUCGACAA |
| | | GGGUGCCAGCGCCCAGAGCUUCAUCGAGCGCAUGACCAACUUCGACAAGAAUCUUCCAAACGAGAAGGUCCUUCCAAAGC |
| | | ACAGCCUCCUCUACGAGUACUUCACCGUCUACAACGAGCUGACCAAGGUCAAGUACGUCACAGAGGGCAUGCGCAAGCCA |
| | | GCCUUCCUCAGCGGUGAGCAGAAGAAGGCCAUCGUCGACCUCCUCUUCAAGACCAACCGCAAGGUCACCGUCAAGCAGCUC |
| | | AAGGAGGACUACUUCAAGAAGAUCGAGUGCUUCGACAGCGUCGAGAUCAGCGGCGUCGAGGACCGCUUCAACGCCAGCCU |
| | | CGGCACCUACCACGACCUCCUCAAGAUCAUCAAGGACAAGGACUUCCUGGACAACGAGGAGAACGAGGACAUCCUCGAGG |
| | | ACAUCGUCCUCACCCUCACCCUCUUCGAGGACAGGGAGAUGAUAGAGGAGCGCCUCAAGACCUACGCCCACCUCUUCGACG |
| | | ACAAGGUCAUGAAGCAGCUCAAGCGCCGCCGCUACACCGGCUGGGGCCGCCUCAGCCGCAAGCUCAUCAAUGGGAUCCGAG |
| | | ACAAGCAGAGCGGCAAGACCAUCCUCGACUUCCUGAAGAGCGACGGCUUCGCCAACCGCAACUUCAUGCAGCUCAUCCACG |
| | | ACGACAGCCUCACCUUCAAGGAGGACAUCCAGAAGGCCCAGGUCAGCGGCCAGGGCGACAGCCUCCACGAGCACAUCGCCA |
| | | AUCUCGCCGGGAGCCCAGCCAUCAAGAAGGGGAUCCUCCAGACCGUCAAGGUCGUCGACGAGCUGGUCAAGGUCAUGGGC |
| | | CGCCACAAGCCAGAGAACAUCGUCAUCGAGAUGGCCAGGGAGAACCAGACCACUCAAAAGGGGCAGAAGAACAGCAGGGA |
| | | GCGCAUGAAGCGCAUCGAGGAGGGCAUCAAGGAGCUGGGCAGCCAGAUCCUCAAGGAGCACCCCGUCGAGAACACUCAAC |
| | | UCCAGAACGAGAAGCUCUACCUCUACUACCUCCAGAAUGGGCGAGACAUGUACGUCGACCAGGAGCUGGACAUCAACCGC |
| | | CUCAGCGACUACGACGUCGACCACAUCGUUCCCCAGAGCUUCCUGAAGGACGACAGCAUCGACAACAAGGUCCUCACCCGA |
| | | AGCGACAAGAACCGCGGCAAGAGCGACAACGUUCCCUCAGAGGAAGUCGUCAAGAAGAUGAAGAACUACUGGCGCCAGCU |
| | | CCUCAACGCCAAGCUCAUCACUCAACGCAAGUUCGACAAUCUCACCAAGGCGGAGCGCGGUGGCCUCAGCGAGCUGGACAA |
| | | GGCCGGGUUCAUCAAGCGCCAGCUCGUCGAGACCCGCCAGAUCACCAAGCACGUCGCCCAGAUCCUCGACAGCCGCAUGAA |
| | | CACCAAGUACGACGAGAACGACAAGCUCAUCAGGGAAGUCAAGGUCAUCACCCUCAAGAGCAAGCUCGUCAGCGACUUCC |
| | | GCAAGGACUUCCAGUUCUACAAGGUCAGGGAGAUCAACAACUACCACCACGCCCACGACGCCUACCUCAACGCUGUGGUU |
| | | GGCACCGCACUGAUCAAGAAGUACCCCAAGCUCGAGAGCGAGUUCGUCUACGGCGACUACAAGGUCUACGACGUCCGCAA |
| | | GAUGAUAGCCAAGAGCGAGCAGGAGAUCGGCAAGGCCACCGCCAAGUACUUCUUCUACAGCAACAUCAUGAACUUCUUCA |
| | | AGACAGAGAUCACCCUCGCCAAUGGUGAGAUCCGCAAGCGCCCCCUCAUCGAGACCAAUGGUGAGACCGGUGAGAUCGUC |
| | | UGGGACAAGGGGCGAGACUUCGCCACCGUCCGCAAGGUCCUCAGCAUGCCCCAGGUGAACAUCGUCAAGAAGACAGAAGU |
| | | CCAGACCGGUGGCUUCAGCAAGGAGAGCAUCCUUCCAAAGCGCAACAGCGACAAGCUCAUCGCCCGCAAGAAGGACUGGG |
| | | ACCCCAAGAAGUACGGUGGCUUCGACAGCCCCACCGUCGCCUACAGCGUCCUCGUCGUCGCCAAGGUCGAGAAGGGGAAG |
| | | AGCAAGAAGCUCAAGAGCGUCAAGGAGCUGCUCGGCAUCACCAUCAUGGAGCGAAGCAGCUUCGAGAAGAACCCCAUCGA |
| | | CUUCCUGGAAGCCAAGGGGUACAAGGAAGUCAAGAAGGACCUCAUCAUCAAGCUUCCAAAGUACAGCCUCUUCGAGCUGG |
| | | AGAAUGGGCGCAAGCGCAUGCUCGCCAGCGCCGGUGAGCUGCAGAAGGGGAACGAGCUGGCACUUCCCUCAAAGUACGUC |
| | | AACUUCCUGUACCUCGCCAGCCACUACGAGAAGCUCAAGGGGAGCCCAGAGGACAACGAGCAGAAGCAGCUCUUCGUCGA |
| | | GCAGCACAAGCACUACCUCGACGAGAUCAUCGAGCAGAUCAGCGAGUUCAGCAAGCGCGUCAUCCUCGCCGACGCCAAUCU |
| | | CGACAAGGUCCUCAGCGCCUACAACAAGCACCGAGACAAGCCCAUCAGGGAGCAGGCCGAGAACAUCAUCCACCUCUUCAC |
| | | CCUCACCAAUCUCGGUGCCCCAGCUGCCUUCAAGUACUUCGACACCACCAUCGACCGCAAGCGCUACACCUCGACUAAGGA |
| | | AGUCCUCGACGCCACCCUCAUCCACCAGAGCAUCACCGGCCUCUACGAGACCCGCAUCGACCUCAGCCAGCUCGGUGGCGA |
| | | CGGUGGUGGCAGCCCCAAGAAGAAGCGCAAGGUCUAG |
| 8 | I-pair depleted | AUGGACAAGAAGUACAGCAUCGGCCUGGACAUCGGCACGAACAGCGUUGGCUGGGCUGUGAUCACGGACGAGUACAAGGU |
| | and/or I-single | UCCCUCAAAGAAGUUCAAGGUGCUGGGCAACACGGACCGGCACAGCAUCAAGAAGAAUCUCAUCGGUGCACUGCUGUUCG |
| | depleted Cas9 | ACAGCGGUGAGACGGCCGAAGCCACGCGGCUGAAGCGGACGGCCCGCCGGCGGUACACGCGGCGGAAGAACCGGAUCUGC |
| | ORF | UACCUGCAGGAGAUCUUCAGCAACGAGAUGGCCAAGGUGGACGACAGCUUCUUCCACCGGCUGGAGGAGAGCUUCCUGGU |
| | | GGAGGAGGACAAGAAGCACGAGCGGCACCCCAUCUUCGGCAACAUCGUGGACGAAGUCGCCUACCACGAGAAGUACCCCA |
| | | CCAUCUACCACCUGCGGAAGAAGCUGGUGGACUCGACUGACAAGGCCGACCUGCGGCUGAUCUACCUGGCACUGGCCCACA |
| | | UGAUAAAGUUCCGGGGCCACUUCCUGAUCGAGGGCGACCUGAACCCUGACAACAGCGACGUGGACAAGCUGUUCAUCCAG |
| | | CUGGUGCAGACCUACAACCAGCUGUUCGAGGAGAACCCCAUCAACGCCAGCGGCGUGGACGCCAAGGCCAUCCUCAGCGCC |
| | | CGCCUCAGCAAGAGCCGGCGGCUGGAGAAUCUCAUCGCCCAGCUUCCAGGUGAGAAGAAGAAUGGGCUGUUCGGCAAUCU |
| | | CAUCGCACUCAGCCUGGGCCUGACUCCCAACUUCAAGAGCAACUUCGACCUGGCCGAGGACGCCAAGCUGCAGCUCAGCAA |
| | | GGACACCUACGACGACGACCUGGACAAUCUCCUGGCCCAGAUCGGCGACCAGUACGCCGACCUGUUCCUGGCUGCCAAGAA |
| | | UCUCAGCGACGCCAUCCUGCUCAGCGACAUCCUGCGGGUGAACACAGAGAUCACGAAGGCCCCCCUCAGCGCCAGCAUGAU |
| | | AAAGCGGUACGACGAGCACCACCAGGACCUGACGCUGCUGAAGGCACUGGUGCGGCAGCAGCUUCCAGAGAAGUACAAGG |
| | | AGAUCUUCUUCGACCAGAGCAAGAAUGGGUACGCCGGGUACAUCGACGGUGGUGCCAGCCAGGAGGAGUUCUACAAGUUC |
| | | AUCAAGCCCAUCCUGGAGAAGAUGGACGGCACAGAGGAGCUGCUGGUGAAGCUGAACAGGGAGGACCUGCUGCGGAAGCA |
| | | GCGGACGUUCGACAAUGGGAGCAUCCCCCACCAGAUCCACCUGGGUGAGCUGCACGCCAUCCUGCGGCGGCAGGAGGACU |
| | | UCUACCCCUUCCUGAAGGACAACAGGGAGAAGAUCGAGAAGAUCCUGACGUUCCGGAUCCCCUACUACGUUGGCCCCCUG |
| | | GCCCGCGGCAACAGCCGGUUCGCCUGGAUGACGCGGAAGAGCGAGGAGACGAUCACUCCCUGGAACUUCGAGGAAGUCGU |
| | | GGACAAGGGUGCCAGCGCCCAGAGCUUCAUCGAGCGGAUGACGAACUUCGACAAGAAUCUUCCAAACGAGAAGGUGCUUC |
| | | CAAAGCACAGCCUGCUGUACGAGUACUUCACGGUGUACAACGAGCUGACGAAGGUGAAGUACGUGACAGAGGGCAUGCGG |
| | | AAGCCCGCCUUCCUCAGCGGUGAGCAGAAGAAGGCCAUCGUGGACCUGCUGUUCAAGACGAACCGGAAGGUGACGGUGAA |
| | | GCAGCUGAAGGAGGACUACUUCAAGAAGAUCGAGUGCUUCGACAGCGUGGAGAUCAGCGGCGUGGAGGACCGGUUCAACG |
| | | CCAGCCUGGGCACCUACCACGACCUGCUGAAGAUCAUCAAGGACAAGGACUUCCUGGACAACGAGGAGAACGAGGACAUC |
| | | CUGGAGGACAUCGUGCUGACGCUGACGCUGUUCGAGGACAGGGAGAUGAUAGAGGAGCGGCUGAAGACCUACGCCCACCU |
| | | GUUCGACGACAAGGUGAUGAAGCAGCUGAAGCGGCGGCGGUACACGGGCUGGGGCCGGCUCAGCCGGAAGCUGAUCAAUG |
| | | GGAUCCGAGACAAGCAGAGCGGCAAGACGAUCCUGGACUUCCUGAAGAGCGACGGCUUCGCCAACCGGAACUUCAUGCAG |
| | | CUGAUCCACGACGACAGCCUGACGUUCAAGGAGGACAUCCAGAAGGCCCAGGUCAGCGGCCAGGGCGACAGCCUGCACGA |
| | | GCACAUCGCCAAUCUCGCCGGGAGCCCCGCCAUCAAGAAGGGGAUCCUGCAGACGGUGAAGGUGGUGGACGAGCUGGUGA |
| | | AGGUGAUGGGCCGGCACAAGCCAGAGAACAUCGUGAUCGAGAUGGCCAGGGAGAACCAGACGACUCAAAAGGGGCAGAAG |
| | | AACAGCAGGGAGCGGAUGAAGCGGAUCGAGGAGGGCAUCAAGGAGCUGGGCAGCCAGAUCCUGAAGGAGCACCCCGUGGA |
| | | GAACACUCAACUGCAGAACGAGAAGCUGUACCUGUACUACCUGCAGAAUGGGCGAGACAUGUACGUGGACCAGGAGCUGG |
| | | ACAUCAACCGGCUCAGCGACUACGACGUGGACCACAUCGUUCCCCAGAGCUUCCUGAAGGACGACAGCAUCGACAACAAG |
| | | GUGCUGACGCGGAGCGACAAGAACCGGGGCAAGAGCGACAACGUUCCCUCAGAGGAAGUCGUGAAGAAGAUGAAGAACUA |
| | | CUGGCGGCAGCUGCUGAACGCCAAGCUGAUCACUCAACGGAAGUUCGACAAUCUCACGAAGGCCGAGCGGGGUGGCCUCA |
| | | GCGAGCUGGACAAGGCCGGGUUCAUCAAGCGGCAGCUGGUGGAGACGCGGCAGAUCACGAAGCACGUGGCCCAGAUCCUG |
| | | GACAGCCGGAUGAACACGAAGUACGACGAGAACGACAAGCUGAUCAGGGAAGUCAAGGUGAUCACGCUGAAGAGCAAGCU |
| | | GGUCAGCGACUUCCGGAAGGACUUCCAGUUCUACAAGGUGAGGGAGAUCAACAACUACCACCACGCCCACGACGCCUACC |
| | | UGAACGCUGUGGUUGGCACGGCACUGAUCAAGAAGUACCCCAAGCUGGAGAGCGAGUUCGUGUACGGCGACUACAAGGUG |
| | | UACGACGUGCGGAAGAUGAUAGCCAAGAGCGAGCAGGAGAUCGGCAAGGCCACGGCCAAGUACUUCUUCUACAGCAACAU |
| | | CAUGAACUUCUUCAAGACAGAGAUCACGCUGGCCAAUGGUGAGAUCCGGAAGCGGCCCCUGAUCGAGACGAAUGGUGAGA |
| | | CGGGUGAGAUCGUGUGGGACAAGGGGCGAGACUUCGCCACGGUGCGGAAGGUGCUCAGCAUGCCCCAGGUGAACAUCGUG |
| | | AAGAAGACAGAAGUCCAGACGGGUGGCUUCAGCAAGGAGAGCAUCCUUCCAAAGCGGAACAGCGACAAGCUGAUCGCCCG |
| | | CAAGAAGGACUGGGACCCCAAGAAGUACGGUGGCUUCGACAGCCCCACCGUGGCCUACAGCGUGCUGGUGGUGGCCAAGG |
| | | UGGAGAAGGGGAAGAGCAAGAAGCUGAAGAGCGUGAAGGAGCUGCUGGGCAUCACGAUCAUGGAGCGGAGCAGCUUCGA |
| | | GAAGAACCCCAUCGACUUCCUGGAAGCCAAGGGGUACAAGGAAGUCAAGAAGGACCUGAUCAUCAAGCUUCCAAAGUACA |
| | | GCCUGUUCGAGCUGGAGAAUGGGCGGAAGCGGAUGCUGGCCAGCGCCGGUGAGCUGCAGAAGGGGAACGAGCUGGCACUU |
| | | CCCUCAAAGUACGUGAACUUCCUGUACCUGGCCAGCCACUACGAGAAGCUGAAGGGGAGCCCAGAGGACAACGAGCAGAA |
| | | GCAGCUGUUCGUGGAGCAGCACAAGCACUACCUGGACGAGAUCAUCGAGCAGAUCAGCGAGUUCAGCAAGCGGGUGAUCC |
| | | UGGCCGACGCCAAUCUCGACAAGGUGCUCAGCGCCUACAACAAGCACCGAGACAAGCCCAUCAGGGAGCAGGCCGAGAAC |
| | | AUCAUCCACCUGUUCACGCUGACGAAUCUCGGUGCCCCCGCUGCCUUCAAGUACUUCGACACGACGAUCGACCGGAAGCGG |
| | | UACACGUCGACUAAGGAAGUCCUGGACGCCACGCUGAUCCACCAGAGCAUCACGGGCCUGUACGAGACGCGGAUCGACCU |
| | | CAGCCAGCUGGGUGGCGACGGUGGUGGCAGCCCCAAGAAGAAGCGGAAGGUGUAG |
| 9 | E-Pair enriched | AUGGACAAGAAGUACAGCAUCGGCCUCGACAUCGGCACCAACAGCGUUGGCUGGGCUGUGAUCACCGACGAGUACAAGGU |
| | Cas9 ORF | UCCCUCAAAGAAGUUCAAGGUCCUCGGCAACACCGACCGCCACAGCAUCAAGAAGAAUCUCAUCGGUGCACUGCUCUUCG |
| | | ACAGCGGUGAGACCGCGGAAGCCACCCGCCUCAAGCGCACCGCCCGCCGCCGCUACACCCGCCGCAAGAACCGCAUCUGCU |
| | | ACCUCCAGGAGAUCUUCAGCAACGAGAUGGCCAAGGUCGACGACAGCUUCUUCCACCGCCUCGAGGAGAGCUUCCUGGUC |
| | | GAGGAGGACAAGAAGCACGAGCGCCACCCCAUCUUCGGCAACAUCGUCGACGAAGUCGCCUACCACGAGAAGUACCCCACC |
| | | AUCUACCACCUGCGGAAGAAGCUCGUCGACUCGACUGACAAGGCCGACCUGCGGCUCAUCUACCUCGCACUGGCCCACAUG |
| | | AUAAAGUUCCGCGGCCACUUCCUGAUCGAGGGCGACCUCAACCCUGACAACAGCGACGUCGACAAGCUCUUCAUCCAGCUC |
| | | GUCCAGACCUACAACCAGCUCUUCGAGGAGAACCCCAUCAACGCCAGCGGCGUCGACGCCAAGGCCAUCCUCAGCGCCCGC |
| | | CUCAGCAAGAGCCGCCGCCUCGAGAAUCUCAUCGCCCAGCUUCCAGGUGAGAAGAAGAAUGGGCUCUUCGGCAAUCUCAU |
| | | CGCACUCAGCCUCGGCCUCACUCCCAACUUCAAGAGCAACUUCGACCUCGCGGAGGACGCCAAGCUCCAGCUCAGCAAGGA |
| | | CACCUACGACGACGACCUCGACAAUCUCCUCGCCCAGAUCGGCGACCAGUACGCCGACCUCUUCCUGGCUGCCAAGAAUCU |
| | | CAGCGACGCCAUCCUCCUCAGCGACAUCCUGCGGGUCAACACAGAGAUCACCAAGGCCCCCCUCAGCGCCAGCAUGAUAAA |
| | | GCGCUACGACGAGCACCACCAGGACCUCACCCUCCUCAAGGCACUGGUCCGCCAGCAGCUUCCAGAGAAGUACAAGGAGAU |
| | | CUUCUUCGACCAGAGCAAGAAUGGGUACGCCGGGUACAUCGACGGUGGUGCCAGCCAGGAGGAGUUCUACAAGUUCAUCA |
| | | AGCCCAUCCUCGAGAAGAUGGACGGCACAGAGGAGCUGCUCGUCAAGCUCAACAGGGAGGACCUCCUGCGGAAGCAGCGC |
| | | ACCUUCGACAAUGGGAGCAUCCCCCACCAGAUCCACCUCGGUGAGCUGCACGCCAUCCUGCGGCGCCAGGAGGACUUCUAC |
| | | CCCUUCCUGAAGGACAACAGGGAGAAGAUCGAGAAGAUCCUCACCUUCCGCAUCCCCUACUACGUUGGCCCCCUCGCCCGC |
| | | GGCAACAGCCGCUUCGCCUGGAUGACCCGCAAGAGCGAGGAGACCAUCACUCCCUGGAACUUCGAGGAAGUCGUCGACAA |
| | | GGGUGCCAGCGCCCAGAGCUUCAUCGAGCGCAUGACCAACUUCGACAAGAAUCUUCCAAACGAGAAGGUCCUUCCAAAGC |
| | | ACAGCCUCCUCUACGAGUACUUCACCGUCUACAACGAGCUGACCAAGGUCAAGUACGUCACAGAGGGCAUGCGCAAGCCA |
| | | GCCUUCCUCAGCGGUGAGCAGAAGAAGGCCAUCGUCGACCUCCUCUUCAAGACCAACCGCAAGGUCACCGUCAAGCAGCUC |
| | | AAGGAGGACUACUUCAAGAAGAUCGAGUGCUUCGACAGCGUCGAGAUCAGCGGCGUCGAGGACCGCUUCAACGCCAGCCU |
| | | CGGCACCUACCACGACCUCCUCAAGAUCAUCAAGGACAAGGACUUCCUGGACAACGAGGAGAACGAGGACAUCCUCGAGG |
| | | ACAUCGUCCUCACCCUCACCCUCUUCGAGGACAGGGAGAUGAUAGAGGAGCGCCUCAAGACCUACGCCCACCUCUUCGACG |
| | | ACAAGGUCAUGAAGCAGCUCAAGCGCCGCCGCUACACCGGCUGGGGCCGCCUCAGCCGCAAGCUCAUCAAUGGGAUCCGAG |
| | | ACAAGCAGAGCGGCAAGACCAUCCUCGACUUCCUGAAGAGCGACGGCUUCGCCAACCGCAACUUCAUGCAGCUCAUCCACG |
| | | ACGACAGCCUCACCUUCAAGGAGGACAUCCAGAAGGCCCAGGUCAGCGGCCAGGGCGACAGCCUCCACGAGCACAUCGCCA |
| | | AUCUCGCCGGGAGCCCAGCCAUCAAGAAGGGGAUCCUCCAGACCGUCAAGGUCGUCGACGAGCUGGUCAAGGUCAUGGGC |
| | | CGCCACAAGCCAGAGAACAUCGUCAUCGAGAUGGCCAGGGAGAACCAGACCACUCAAAAGGGGCAGAAGAACAGCAGGGA |
| | | GCGCAUGAAGCGCAUCGAGGAGGGCAUCAAGGAGCUGGGCAGCCAGAUCCUCAAGGAGCACCCCGUCGAGAACACUCAAC |
| | | UCCAGAACGAGAAGCUCUACCUCUACUACCUCCAGAAUGGGCGAGACAUGUACGUCGACCAGGAGCUGGACAUCAACCGC |
| | | CUCAGCGACUACGACGUCGACCACAUCGUUCCCCAGAGCUUCCUGAAGGACGACAGCAUCGACAACAAGGUCCUCACCCGA |
| | | AGCGACAAGAACCGCGGCAAGAGCGACAACGUUCCCUCAGAGGAAGUCGUCAAGAAGAUGAAGAACUACUGGCGCCAGCU |
| | | CCUCAACGCCAAGCUCAUCACUCAACGCAAGUUCGACAAUCUCACCAAGGCGGAGCGCGGUGGCCUCAGCGAGCUGGACAA |
| | | GGCCGGGUUCAUCAAGCGCCAGCUCGUCGAGACCCGCCAGAUCACCAAGCACGUCGCCCAGAUCCUCGACAGCCGCAUGAA |
| | | CACCAAGUACGACGAGAACGACAAGCUCAUCAGGGAAGUCAAGGUCAUCACCCUCAAGAGCAAGCUCGUCAGCGACUUCC |
| | | GCAAGGACUUCCAGUUCUACAAGGUCAGGGAGAUCAACAACUACCACCACGCCCACGACGCCUACCUCAACGCUGUGGUU |
| | | GGCACCGCACUGAUCAAGAAGUACCCCAAGCUCGAGAGCGAGUUCGUCUACGGCGACUACAAGGUCUACGACGUCCGCAA |
| | | GAUGAUAGCCAAGAGCGAGCAGGAGAUCGGCAAGGCCACCGCCAAGUACUUCUUCUACAGCAACAUCAUGAACUUCUUCA |
| | | AGACAGAGAUCACCCUCGCCAAUGGUGAGAUCCGCAAGCGCCCCCUCAUCGAGACCAAUGGUGAGACCGGUGAGAUCGUC |
| | | UGGGACAAGGGGCGAGACUUCGCCACCGUCCGCAAGGUCCUCAGCAUGCCCCAGGUGAACAUCGUCAAGAAGACAGAAGU |
| | | CCAGACCGGUGGCUUCAGCAAGGAGAGCAUCCUUCCAAAGCGCAACAGCGACAAGCUCAUCGCCCGCAAGAAGGACUGGG |
| | | ACCCCAAGAAGUACGGUGGCUUCGACAGCCCCACCGUCGCCUACAGCGUCCUCGUCGUCGCCAAGGUCGAGAAGGGGAAG |
| | | AGCAAGAAGCUCAAGAGCGUCAAGGAGCUGCUCGGCAUCACCAUCAUGGAGCGAAGCAGCUUCGAGAAGAACCCCAUCGA |
| | | CUUCCUGGAAGCCAAGGGGUACAAGGAAGUCAAGAAGGACCUCAUCAUCAAGCUUCCAAAGUACAGCCUCUUCGAGCUGG |
| | | AGAAUGGGCGCAAGCGCAUGCUCGCCAGCGCCGGUGAGCUGCAGAAGGGGAACGAGCUGGCACUUCCCUCAAAGUACGUC |
| | | AACUUCCUGUACCUCGCCAGCCACUACGAGAAGCUCAAGGGGAGCCCAGAGGACAACGAGCAGAAGCAGCUCUUCGUCGA |
| | | GCAGCACAAGCACUACCUCGACGAGAUCAUCGAGCAGAUCAGCGAGUUCAGCAAGCGCGUCAUCCUCGCCGACGCCAAUCU |
| | | CGACAAGGUCCUCAGCGCCUACAACAAGCACCGAGACAAGCCCAUCAGGGAGCAGGCCGAGAACAUCAUCCACCUCUUCAC |
| | | CCUCACCAAUCUCGGUGCCCCAGCUGCCUUCAAGUACUUCGACACCACCAUCGACCGCAAGCGCUACACCUCGACUAAGGA |
| | | AGUCCUCGACGCCACCCUCAUCCACCAGAGCAUCACCGGCCUCUACGAGACCCGCAUCGACCUCAGCCAGCUCGGUGGCGA |
| | | CGGUGGUGGCAGCCCCAAGAAGAAGCGCAAGGUCUAG |
| 10 | E-pair and E- | AUGGACAAGAAGUACAGCAUCGGCCUCGACAUCGGCACCAACAGCGUCGGCUGGGCCGUCAUCACCGACGAGUACAAGGU |
| | single enriched | CCCCAGCAAGAAGUUCAAGGUCCUCGGCAACACCGACCGCCACAGCAUCAAGAAGAACCUCAUCGGCGCCCUCCUCUUCGA |
| | Cas9 ORF | CAGCGGCGAGACCGCCGAGGCCACCCGCCUCAAGCGCACCGCCCGCCGCCGCUACACCCGCCGCAAGAACCGCAUCUGCUA |
| | | CCUCCAGGAGAUCUUCAGCAACGAGAUGGCCAAGGUCGACGACAGCUUCUUCCACCGCCUCGAGGAGAGCUUCCUCGUCG |
| | | AGGAGGACAAGAAGCACGAGCGCCACCCCAUCUUCGGCAACAUCGUCGACGAGGUCGCCUACCACGAGAAGUACCCCACCA |
| | | UCUACCACCUCCGCAAGAAGCUCGUCGACAGCACCGACAAGGCCGACCUCCGCCUCAUCUACCUCGCCCUCGCCCACAUGA |
| | | UCAAGUUCCGCGGCCACUUCCUCAUCGAGGGCGACCUCAACCCCGACAACAGCGACGUCGACAAGCUCUUCAUCCAGCUCG |
| | | UCCAGACCUACAACCAGCUCUUCGAGGAGAACCCCAUCAACGCCAGCGGCGUCGACGCCAAGGCCAUCCUCAGCGCCCGCC |
| | | UCAGCAAGAGCCGCCGCCUCGAGAACCUCAUCGCCCAGCUCCCCGGCGAGAAGAAGAACGGCCUCUUCGGCAACCUCAUCG |
| | | CCCUCAGCCUCGGCCUCACCCCCAACUUCAAGAGCAACUUCGACCUCGCCGAGGACGCCAAGCUCCAGCUCAGCAAGGACA |
| | | CCUACGACGACGACCUCGACAACCUCCUCGCCCAGAUCGGCGACCAGUACGCCGACCUCUUCCUCGCCGCCAAGAACCUCA |
| | | GCGACGCCAUCCUCCUCAGCGACAUCCUCCGCGUCAACACCGAGAUCACCAAGGCCCCCCUCAGCGCCAGCAUGAUCAAGC |
| | | GCUACGACGAGCACCACCAGGACCUCACCCUCCUCAAGGCCCUCGUCCGCCAGCAGCUCCCCGAGAAGUACAAGGAGAUCU |
| | | UCUUCGACCAGAGCAAGAACGGCUACGCCGGCUACAUCGACGGCGGCGCCAGCCAGGAGGAGUUCUACAAGUUCAUCAAG |
| | | CCCAUCCUCGAGAAGAUGGACGGCACCGAGGAGCUCCUCGUCAAGCUCAACCGCGAGGACCUCCUCCGCAAGCAGCGCACC |
| | | UUCGACAACGGCAGCAUCCCCCACCAGAUCCACCUCGGCGAGCUCCACGCCAUCCUCCGCCGCCAGGAGGACUUCUACCCC |
| | | UUCCUCAAGGACAACCGCGAGAAGAUCGAGAAGAUCCUCACCUUCCGCAUCCCCUACUACGUCGGCCCCCUCGCCCGCGGC |
| | | AACAGCCGCUUCGCCUGGAUGACCCGCAAGAGCGAGGAGACCAUCACCCCCUGGAACUUCGAGGAGGUCGUCGACAAGGG |
| | | CGCCAGCGCCCAGAGCUUCAUCGAGCGCAUGACCAACUUCGACAAGAACCUCCCCAACGAGAAGGUCCUCCCCAAGCACAG |
| | | CCUCCUCUACGAGUACUUCACCGUCUACAACGAGCUCACCAAGGUCAAGUACGUCACCGAGGGCAUGCGCAAGCCCGCCUU |
| | | CCUCAGCGGCGAGCAGAAGAAGGCCAUCGUCGACCUCCUCUUCAAGACCAACCGCAAGGUCACCGUCAAGCAGCUCAAGGA |
| | | GGACUACUUCAAGAAGAUCGAGUGCUUCGACAGCGUCGAGAUCAGCGGCGUCGAGGACCGCUUCAACGCCAGCCUCGGCA |
| | | CCUACCACGACCUCCUCAAGAUCAUCAAGGACAAGGACUUCCUCGACAACGAGGAGAACGAGGACAUCCUCGAGGACAUC |
| | | GUCCUCACCCUCACCCUCUUCGAGGACCGCGAGAUGAUCGAGGAGCGCCUCAAGACCUACGCCCACCUCUUCGACGACAAG |
| | | GUCAUGAAGCAGCUCAAGCGCCGCCGCUACACCGGCUGGGGCCGCCUCAGCCGCAAGCUCAUCAACGGCAUCCGCGACAAG |
| | | CAGAGCGGCAAGACCAUCCUCGACUUCCUCAAGAGCGACGGCUUCGCCAACCGCAACUUCAUGCAGCUCAUCCACGACGAC |
| | | AGCCUCACCUUCAAGGAGGACAUCCAGAAGGCCCAGGUCAGCGGCCAGGGCGACAGCCUCCACGAGCACAUCGCCAACCUC |
| | | GCCGGCAGCCCCGCCAUCAAGAAGGGCAUCCUCCAGACCGUCAAGGUCGUCGACGAGCUCGUCAAGGUCAUGGGCCGCCAC |
| | | AAGCCCGAGAACAUCGUCAUCGAGAUGGCCCGCGAGAACCAGACCACCCAGAAGGGCCAGAAGAACAGCCGCGAGCGCAU |
| | | GAAGCGCAUCGAGGAGGGCAUCAAGGAGCUCGGCAGCCAGAUCCUCAAGGAGCACCCCGUCGAGAACACCCAGCUCCAGA |
| | | ACGAGAAGCUCUACCUCUACUACCUCCAGAACGGCCGCGACAUGUACGUCGACCAGGAGCUCGACAUCAACCGCCUCAGCG |
| | | ACUACGACGUCGACCACAUCGUCCCCCAGAGCUUCCUCAAGGACGACAGCAUCGACAACAAGGUCCUCACCCGCAGCGACA |
| | | AGAACCGCGGCAAGAGCGACAACGUCCCCAGCGAGGAGGUCGUCAAGAAGAUGAAGAACUACUGGCGCCAGCUCCUCAAC |
| | | GCCAAGCUCAUCACCCAGCGCAAGUUCGACAACCUCACCAAGGCCGAGCGCGGCGGCCUCAGCGAGCUCGACAAGGCCGGC |
| | | UUCAUCAAGCGCCAGCUCGUCGAGACCCGCCAGAUCACCAAGCACGUCGCCCAGAUCCUCGACAGCCGCAUGAACACCAAG |
| | | UACGACGAGAACGACAAGCUCAUCCGCGAGGUCAAGGUCAUCACCCUCAAGAGCAAGCUCGUCAGCGACUUCCGCAAGGA |
| | | CUUCCAGUUCUACAAGGUCCGCGAGAUCAACAACUACCACCACGCCCACGACGCCUACCUCAACGCCGUCGUCGGCACCGC |
| | | CCUCAUCAAGAAGUACCCCAAGCUCGAGAGCGAGUUCGUCUACGGCGACUACAAGGUCUACGACGUCCGCAAGAUGAUCG |
| | | CCAAGAGCGAGCAGGAGAUCGGCAAGGCCACCGCCAAGUACUUCUUCUACAGCAACAUCAUGAACUUCUUCAAGACCGAG |
| | | AUCACCCUCGCCAACGGCGAGAUCCGCAAGCGCCCCCUCAUCGAGACCAACGGCGAGACCGGCGAGAUCGUCUGGGACAAG |
| | | GGCCGCGACUUCGCCACCGUCCGCAAGGUCCUCAGCAUGCCCCAGGUCAACAUCGUCAAGAAGACCGAGGUCCAGACCGGC |
| | | GGCUUCAGCAAGGAGAGCAUCCUCCCCAAGCGCAACAGCGACAAGCUCAUCGCCCGCAAGAAGGACUGGGACCCCAAGAA |
| | | GUACGGCGGCUUCGACAGCCCCACCGUCGCCUACAGCGUCCUCGUCGUCGCCAAGGUCGAGAAGGGCAAGAGCAAGAAGC |
| | | UCAAGAGCGUCAAGGAGCUCCUCGGCAUCACCAUCAUGGAGCGCAGCAGCUUCGAGAAGAACCCCAUCGACUUCCUCGAG |
| | | GCCAAGGGCUACAAGGAGGUCAAGAAGGACCUCAUCAUCAAGCUCCCCAAGUACAGCCUCUUCGAGCUCGAGAACGGCCG |
| | | CAAGCGCAUGCUCGCCAGCGCCGGCGAGCUCCAGAAGGGCAACGAGCUCGCCCUCCCCAGCAAGUACGUCAACUUCCUCUA |
| | | CCUCGCCAGCCACUACGAGAAGCUCAAGGGCAGCCCCGAGGACAACGAGCAGAAGCAGCUCUUCGUCGAGCAGCACAAGCA |
| | | CUACCUCGACGAGAUCAUCGAGCAGAUCAGCGAGUUCAGCAAGCGCGUCAUCCUCGCCGACGCCAACCUCGACAAGGUCCU |
| | | CAGCGCCUACAACAAGCACCGCGACAAGCCCAUCCGCGAGCAGGCCGAGAACAUCAUCCACCUCUUCACCCUCACCAACCU |
| | | CGGCGCCCCCGCCGCCUUCAAGUACUUCGACACCACCAUCGACCGCAAGCGCUACACCAGCACCAAGGAGGUCCUCGACGC |
| | | CACCCUCAUCCACCAGAGCAUCACCGGCCUCUACGAGACCCGCAUCGACCUCAGCCAGCUCGGCGGCGACGGCGGCGGCAG |
| | | CCCCAAGAAGAAGCGCAAGGUCUAG |
| 11 | E-single depleted | AUGGACAAAAAAUACUCAAUAGGCCUCGACAUAGGCACCAACUCAGUCGGCUGGGCCGUCAUAACCGACGAGUACAAAGU |
| | Cas9 ORF | CCCCUCAAAAAAAUUCAAAGUCCUCGGCAACACCGACAGGCACUCAAUAAAAAAAAACCUCAUAGGCGCCCUCCUCUUCGA |
| | | CUCAGGCGAGACCGCCGAGGCCACCAGGCUCAAAAGGACCGCCAGGAGGAGGUACACCAGGAGGAAAAACAGGAUAUGCU |
| | | ACCUCCAGGAGAUAUUCUCAAACGAGAUGGCCAAAGUCGACGACUCAUUCUUCCACAGGCUCGAGGAGUCAUUCCUCGUC |
| | | GAGGAGGACAAAAAACACGAGAGGCACCCCAUAUUCGGCAACAUAGUCGACGAGGUCGCCUACCACGAGAAAUACCCCAC |
| | | CAUAUACCACCUCAGGAAAAAACUCGUCGACUCAACCGACAAAGCCGACCUCAGGCUCAUAUACCUCGCCCUCGCCCACAU |
| | | GAUAAAAUUCAGGGGCCACUUCCUCAUAGAGGGCGACCUCAACCCCGACAACUCAGACGUCGACAAACUCUUCAUACAGC |
| | | UCGUCCAGACCUACAACCAGCUCUUCGAGGAGAACCCCAUAAACGCCUCAGGCGUCGACGCCAAAGCCAUACUCUCAGCCA |
| | | GGCUCUCAAAAUCAAGGAGGCUCGAGAACCUCAUAGCCCAGCUCCCCGGCGAGAAAAAAAACGGCCUCUUCGGCAACCUC |
| | | AUAGCCCUCUCACUCGGCCUCACCCCCAACUUCAAAUCAAACUUCGACCUCGCCGAGGACGCCAAACUCCAGCUCUCAAAA |
| | | GACACCUACGACGACGACCUCGACAACCUCCUCGCCCAGAUAGGCGACCAGUACGCCGACCUCUUCCUCGCCGCCAAAAAC |
| | | CUCUCAGACGCCAUACUCCUCUCAGACAUACUCAGGGUCAACACCGAGAUAACCAAAGCCCCCCUCUCAGCCUCAAUGAUA |
| | | AAAAGGUACGACGAGCACCACCAGGACCUCACCCUCCUCAAAGCCCUCGUCAGGCAGCAGCUCCCCGAGAAAUACAAAGAG |
| | | AUAUUCUUCGACCAGUCAAAAAACGGCUACGCCGGCUACAUAGACGGCGGCGCCUCACAGGAGGAGUUCUACAAAUUCAU |
| | | AAAACCCAUACUCGAGAAAAUGGACGGCACCGAGGAGCUCCUCGUCAAACUCAACAGGGAGGACCUCCUCAGGAAACAGA |
| | | GGACCUUCGACAACGGCUCAAUACCCCACCAGAUACACCUCGGCGAGCUCCACGCCAUACUCAGGAGGCAGGAGGACUUCU |
| | | ACCCCUUCCUCAAAGACAACAGGGAGAAAAUAGAGAAAAUACUCACCUUCAGGAUACCCUACUACGUCGGCCCCCUCGCCA |
| | | GGGGCAACUCAAGGUUCGCCUGGAUGACCAGGAAAUCAGAGGAGACCAUAACCCCCUGGAACUUCGAGGAGGUCGUCGAC |
| | | AAAGGCGCCUCAGCCCAGUCAUUCAUAGAGAGGAUGACCAACUUCGACAAAAACCUCCCCAACGAGAAAGUCCUCCCCAA |
| | | ACACUCACUCCUCUACGAGUACUUCACCGUCUACAACGAGCUCACCAAAGUCAAAUACGUCACCGAGGGCAUGAGGAAAC |
| | | CCGCCUUCCUCUCAGGCGAGCAGAAAAAAGCCAUAGUCGACCUCCUCUUCAAAACCAACAGGAAAGUCACCGUCAAACAGC |
| | | UCAAAGAGGACUACUUCAAAAAAAUAGAGUGCUUCGACUCAGUCGAGAUAUCAGGCGUCGAGGACAGGUUCAACGCCUCA |
| | | CUCGGCACCUACCACGACCUCCUCAAAAUAAUAAAAGACAAAGACUUCCUCGACAACGAGGAGAACGAGGACAUACUCGA |
| | | GGACAUAGUCCUCACCCUCACCCUCUUCGAGGACAGGGAGAUGAUAGAGGAGAGGCUCAAAACCUACGCCCACCUCUUCG |
| | | ACGACAAAGUCAUGAAACAGCUCAAAAGGAGGAGGUACACCGGCUGGGGCAGGCUCUCAAGGAAACUCAUAAACGGCAUA |
| | | AGGGACAAACAGUCAGGCAAAACCAUACUCGACUUCCUCAAAUCAGACGGCUUCGCCAACAGGAACUUCAUGCAGCUCAU |
| | | ACACGACGACUCACUCACCUUCAAAGAGGACAUACAGAAAGCCCAGGUCUCAGGCCAGGGCGACUCACUCCACGAGCACAU |
| | | AGCCAACCUCGCCGGCUCACCCGCCAUAAAAAAAGGCAUACUCCAGACCGUCAAAGUCGUCGACGAGCUCGUCAAAGUCAU |
| | | GGGCAGGCACAAACCCGAGAACAUAGUCAUAGAGAUGGCCAGGGAGAACCAGACCACCCAGAAAGGCCAGAAAAACUCAA |
| | | GGGAGAGGAUGAAAAGGAUAGAGGAGGGCAUAAAAGAGCUCGGCUCACAGAUACUCAAAGAGCACCCCGUCGAGAACACC |
| | | CAGCUCCAGAACGAGAAACUCUACCUCUACUACCUCCAGAACGGCAGGGACAUGUACGUCGACCAGGAGCUCGACAUAAA |
| | | CAGGCUCUCAGACUACGACGUCGACCACAUAGUCCCCCAGUCAUUCCUCAAAGACGACUCAAUAGACAACAAAGUCCUCAC |
| | | CAGGUCAGACAAAAACAGGGGCAAAUCAGACAACGUCCCCUCAGAGGAGGUCGUCAAAAAAAUGAAAAACUACUGGAGGC |
| | | AGCUCCUCAACGCCAAACUCAUAACCCAGAGGAAAUUCGACAACCUCACCAAAGCCGAGAGGGGCGGCCUCUCAGAGCUCG |
| | | ACAAAGCCGGCUUCAUAAAAAGGCAGCUCGUCGAGACCAGGCAGAUAACCAAACACGUCGCCCAGAUACUCGACUCAAGG |
| | | AUGAACACCAAAUACGACGAGAACGACAAACUCAUAAGGGAGGUCAAAGUCAUAACCCUCAAAUCAAAACUCGUCUCAGA |
| | | CUUCAGGAAAGACUUCCAGUUCUACAAAGUCAGGGAGAUAAACAACUACCACCACGCCCACGACGCCUACCUCAACGCCGU |
| | | CGUCGGCACCGCCCUCAUAAAAAAAUACCCCAAACUCGAGUCAGAGUUCGUCUACGGCGACUACAAAGUCUACGACGUCA |
| | | GGAAAAUGAUAGCCAAAUCAGAGCAGGAGAUAGGCAAAGCCACCGCCAAAUACUUCUUCUACUCAAACAUAAUGAACUUC |
| | | UUCAAAACCGAGAUAACCCUCGCCAACGGCGAGAUAAGGAAAAGGCCCCUCAUAGAGACCAACGGCGAGACCGGCGAGAU |
| | | AGUCUGGGACAAAGGCAGGGACUUCGCCACCGUCAGGAAAGUCCUCUCAAUGCCCCAGGUCAACAUAGUCAAAAAAACCG |
| | | AGGUCCAGACCGGCGGCUUCUCAAAAGAGUCAAUACUCCCCAAAAGGAACUCAGACAAACUCAUAGCCAGGAAAAAAGAC |
| | | UGGGACCCCAAAAAAUACGGCGGCUUCGACUCACCCACCGUCGCCUACUCAGUCCUCGUCGUCGCCAAAGUCGAGAAAGGC |
| | | AAAUCAAAAAAACUCAAAUCAGUCAAAGAGCUCCUCGGCAUAACCAUAAUGGAGAGGUCAUCAUUCGAGAAAAACCCCAU |
| | | AGACUUCCUCGAGGCCAAAGGCUACAAAGAGGUCAAAAAAGACCUCAUAAUAAAACUCCCCAAAUACUCACUCUUCGAGC |
| | | UCGAGAACGGCAGGAAAAGGAUGCUCGCCUCAGCCGGCGAGCUCCAGAAAGGCAACGAGCUCGCCCUCCCCUCAAAAUAC |
| | | GUCAACUUCCUCUACCUCGCCUCACACUACGAGAAACUCAAAGGCUCACCCGAGGACAACGAGCAGAAACAGCUCUUCGUC |
| | | GAGCAGCACAAACACUACCUCGACGAGAUAAUAGAGCAGAUAUCAGAGUUCUCAAAAAGGGUCAUACUCGCCGACGCCAA |
| | | CCUCGACAAAGUCCUCUCAGCCUACAACAAACACAGGGACAAACCCAUAAGGGAGCAGGCCGAGAACAUAAUACACCUCU |
| | | UCACCCUCACCAACCUCGGCGCCCCCGCCGCCUUCAAAUACUUCGACACCACCAUAGACAGGAAAAGGUACACCUCAACCA |
| | | AAGAGGUCCUCGACGCCACCCUCAUACACCAGUCAAUAACCGGCCUCUACGAGACCAGGAUAGACCUCUCACAGCUCGGCG |
| | | GCGACGGCGGCGGCUCACCCAAAAAAAAAAGGAAAGUCUAG |
| 12 | I-single enriched | AUGGACAAAAAAUACAGCAUCGGCCUGGACAUCGGCACGAACAGCGUGGGCUGGGCCGUGAUCACGGACGAGUACAAAGU |
| | Cas9 ORF | GCCCAGCAAAAAAUUCAAAGUGCUGGGCAACACGGACCGGCACAGCAUCAAAAAAAACCUGAUCGGCGCCCUGCUGUUCG |
| | | ACAGCGGCGAGACGGCCGAGGCCACGCGGCUGAAACGGACGGCCCGGCGGCGGUACACGCGGCGGAAAAACCGGAUCUGC |
| | | UACCUGCAGGAGAUCUUCAGCAACGAGAUGGCCAAAGUGGACGACAGCUUCUUCCACCGGCUGGAGGAGAGCUUCCUGGU |
| | | GGAGGAGGACAAAAAACACGAGCGGCACCCCAUCUUCGGCAACAUCGUGGACGAGGUGGCCUACCACGAGAAAUACCCCA |
| | | CGAUCUACCACCUGCGGAAAAAACUGGUGGACAGCACGGACAAAGCCGACCUGCGGCUGAUCUACCUGGCCCUGGCCCACA |
| | | UGAUCAAAUUCCGGGGCCACUUCCUGAUCGAGGGCGACCUGAACCCCGACAACAGCGACGUGGACAAACUGUUCAUCCAG |
| | | CUGGUGCAGACGUACAACCAGCUGUUCGAGGAGAACCCCAUCAACGCCAGCGGCGUGGACGCCAAAGCCAUCCUGAGCGCC |
| | | CGGCUGAGCAAAAGCCGGCGGCUGGAGAACCUGAUCGCCCAGCUGCCCGGCGAGAAAAAAAACGGCCUGUUCGGCAACCU |
| | | GAUCGCCCUGAGCCUGGGCCUGACGCCCAACUUCAAAAGCAACUUCGACCUGGCCGAGGACGCCAAACUGCAGCUGAGCAA |
| | | AGACACGUACGACGACGACCUGGACAACCUGCUGGCCCAGAUCGGCGACCAGUACGCCGACCUGUUCCUGGCCGCCAAAAA |
| | | CCUGAGCGACGCCAUCCUGCUGAGCGACAUCCUGCGGGUGAACACGGAGAUCACGAAAGCCCCCCUGAGCGCCAGCAUGAU |
| | | CAAACGGUACGACGAGCACCACCAGGACCUGACGCUGCUGAAAGCCCUGGUGCGGCAGCAGCUGCCCGAGAAAUACAAAG |
| | | AGAUCUUCUUCGACCAGAGCAAAAACGGCUACGCCGGCUACAUCGACGGCGGCGCCAGCCAGGAGGAGUUCUACAAAUUC |
| | | AUCAAACCCAUCCUGGAGAAAAUGGACGGCACGGAGGAGCUGCUGGUGAAACUGAACCGGGAGGACCUGCUGCGGAAACA |
| | | GCGGACGUUCGACAACGGCAGCAUCCCCCACCAGAUCCACCUGGGCGAGCUGCACGCCAUCCUGCGGCGGCAGGAGGACUU |
| | | CUACCCCUUCCUGAAAGACAACCGGGAGAAAAUCGAGAAAAUCCUGACGUUCCGGAUCCCCUACUACGUGGGCCCCCUGGC |
| | | CCGGGGCAACAGCCGGUUCGCCUGGAUGACGCGGAAAAGCGAGGAGACGAUCACGCCCUGGAACUUCGAGGAGGUGGUGG |
| | | ACAAAGGCGCCAGCGCCCAGAGCUUCAUCGAGCGGAUGACGAACUUCGACAAAAACCUGCCCAACGAGAAAGUGCUGCCC |
| | | AAACACAGCCUGCUGUACGAGUACUUCACGGUGUACAACGAGCUGACGAAAGUGAAAUACGUGACGGAGGGCAUGCGGAA |
| | | ACCCGCCUUCCUGAGCGGCGAGCAGAAAAAAGCCAUCGUGGACCUGCUGUUCAAAACGAACCGGAAAGUGACGGUGAAAC |
| | | AGCUGAAAGAGGACUACUUCAAAAAAAUCGAGUGCUUCGACAGCGUGGAGAUCAGCGGCGUGGAGGACCGGUUCAACGCC |
| | | AGCCUGGGCACGUACCACGACCUGCUGAAAAUCAUCAAAGACAAAGACUUCCUGGACAACGAGGAGAACGAGGACAUCCU |
| | | GGAGGACAUCGUGCUGACGCUGACGCUGUUCGAGGACCGGGAGAUGAUCGAGGAGCGGCUGAAAACGUACGCCCACCUGU |
| | | UCGACGACAAAGUGAUGAAACAGCUGAAACGGCGGCGGUACACGGGCUGGGGCCGGCUGAGCCGGAAACUGAUCAACGGC |
| | | AUCCGGGACAAACAGAGCGGCAAAACGAUCCUGGACUUCCUGAAAAGCGACGGCUUCGCCAACCGGAACUUCAUGCAGCU |
| | | GAUCCACGACGACAGCCUGACGUUCAAAGAGGACAUCCAGAAAGCCCAGGUGAGCGGCCAGGGCGACAGCCUGCACGAGC |
| | | ACAUCGCCAACCUGGCCGGCAGCCCCGCCAUCAAAAAAGGCAUCCUGCAGACGGUGAAAGUGGUGGACGAGCUGGUGAAA |
| | | GUGAUGGGCCGGCACAAACCCGAGAACAUCGUGAUCGAGAUGGCCCGGGAGAACCAGACGACGCAGAAAGGCCAGAAAAA |
| | | CAGCCGGGAGCGGAUGAAACGGAUCGAGGAGGGCAUCAAAGAGCUGGGCAGCCAGAUCCUGAAAGAGCACCCCGUGGAGA |
| | | ACACGCAGCUGCAGAACGAGAAACUGUACCUGUACUACCUGCAGAACGGCCGGGACAUGUACGUGGACCAGGAGCUGGAC |
| | | AUCAACCGGCUGAGCGACUACGACGUGGACCACAUCGUGCCCCAGAGCUUCCUGAAAGACGACAGCAUCGACAACAAAGU |
| | | GCUGACGCGGAGCGACAAAAACCGGGGCAAAAGCGACAACGUGCCCAGCGAGGAGGUGGUGAAAAAAAUGAAAAACUACU |
| | | GGCGGCAGCUGCUGAACGCCAAACUGAUCACGCAGCGGAAAUUCGACAACCUGACGAAAGCCGAGCGGGGCGGCCUGAGC |
| | | GAGCUGGACAAAGCCGGCUUCAUCAAACGGCAGCUGGUGGAGACGCGGCAGAUCACGAAACACGUGGCCCAGAUCCUGGA |
| | | CAGCCGGAUGAACACGAAAUACGACGAGAACGACAAACUGAUCCGGGAGGUGAAAGUGAUCACGCUGAAAAGCAAACUGG |
| | | UGAGCGACUUCCGGAAAGACUUCCAGUUCUACAAAGUGCGGGAGAUCAACAACUACCACCACGCCCACGACGCCUACCUG |
| | | AACGCCGUGGUGGGCACGGCCCUGAUCAAAAAAUACCCCAAACUGGAGAGCGAGUUCGUGUACGGCGACUACAAAGUGUA |
| | | CGACGUGCGGAAAAUGAUCGCCAAAAGCGAGCAGGAGAUCGGCAAAGCCACGGCCAAAUACUUCUUCUACAGCAACAUCA |
| | | UGAACUUCUUCAAAACGGAGAUCACGCUGGCCAACGGCGAGAUCCGGAAACGGCCCCUGAUCGAGACGAACGGCGAGACG |
| | | GGCGAGAUCGUGUGGGACAAAGGCCGGGACUUCGCCACGGUGCGGAAAGUGCUGAGCAUGCCCCAGGUGAACAUCGUGAA |
| | | AAAAACGGAGGUGCAGACGGGCGGCUUCAGCAAAGAGAGCAUCCUGCCCAAACGGAACAGCGACAAACUGAUCGCCCGGA |
| | | AAAAAGACUGGGACCCCAAAAAAUACGGCGGCUUCGACAGCCCCACGGUGGCCUACAGCGUGCUGGUGGUGGCCAAAGUG |
| | | GAGAAAGGCAAAAGCAAAAAACUGAAAAGCGUGAAAGAGCUGCUGGGCAUCACGAUCAUGGAGCGGAGCAGCUUCGAGA |
| | | AAAACCCCAUCGACUUCCUGGAGGCCAAAGGCUACAAAGAGGUGAAAAAAGACCUGAUCAUCAAACUGCCCAAAUACAGC |
| | | CUGUUCGAGCUGGAGAACGGCCGGAAACGGAUGCUGGCCAGCGCCGGCGAGCUGCAGAAAGGCAACGAGCUGGCCCUGCC |
| | | CAGCAAAUACGUGAACUUCCUGUACCUGGCCAGCCACUACGAGAAACUGAAAGGCAGCCCCGAGGACAACGAGCAGAAAC |
| | | AGCUGUUCGUGGAGCAGCACAAACACUACCUGGACGAGAUCAUCGAGCAGAUCAGCGAGUUCAGCAAACGGGUGAUCCUG |
| | | GCCGACGCCAACCUGGACAAAGUGCUGAGCGCCUACAACAAACACCGGGACAAACCCAUCCGGGAGCAGGCCGAGAACAUC |
| | | AUCCACCUGUUCACGCUGACGAACCUGGGCGCCCCCGCCGCCUUCAAAUACUUCGACACGACGAUCGACCGGAAACGGUAC |
| | | ACGAGCACGAAAGAGGUGCUGGACGCCACGCUGAUCCACCAGAGCAUCACGGGCCUGUACGAGACGCGGAUCGACCUGAG |
| | | CCAGCUGGGCGGCGACGGCGGCGGCAGCCCCAAAAAAAAACGGAAAGUGUAG |
| 13 | E-pair depleted | AUGGAUAAAAAAUAUUCAAUAGGAUUAGAUAUAGGAACAAAUUCAGUAGGAUGGGCAGUAAUAACAGAUGAAUAUAAAG |
| | Cas9 ORF | UACCAUCAAAAAAAUUUAAAGUAUUAGGAAAUACAGAUAGACAUUCAAUAAAAAAAAAUUUAAUAGGAGCAUUAUUAUU |
| | | UGAUUCAGGAGAAACAGCAGAAGCAACAAGAUUAAAAAGAACAGCAAGAAGAAGAUAUACAAGAAGAAAAAAUAGAAUA |
| | | UGUUAUUUACAAGAAAUAUUUUCAAAUGAAAUGGCAAAAGUAGAUGAUUCAUUUUUUCAUAGAUUAGAAGAAUCAUUUU |
| | | UAGUAGAAGAAGAUAAAAAACAUGAAAGACAUCCAAUAUUUGGAAAUAUAGUAGAUGAAGUAGCAUAUCAUGAAAAAUA |
| | | UCCAACAAUAUAUCAUUUAAGAAAAAAAUUAGUAGAUUCAACAGAUAAAGCAGAUUUAAGAUUAAUAUAUUUAGCAUUA |
| | | GCACAUAUGAUAAAAUUUAGAGGACAUUUUUUAAUAGAAGGAGAUUUAAAUCCAGAUAAUUCAGAUGUAGAUAAAUUAU |
| | | UUAUACAAUUAGUACAAACAUAUAAUCAAUUAUUUGAAGAAAAUCCAAUAAAUGCAUCAGGAGUAGAUGCAAAAGCAAU |
| | | AUUAUCAGCAAGAUUAUCAAAAUCAAGAAGAUUAGAAAAUUUAAUAGCACAAUUACCAGGAGAAAAAAAAAAUGGAUUA |
| | | UUUGGAAAUUUAAUAGCAUUAUCAUUAGGAUUAACACCAAAUUUUAAAUCAAAUUUUGAUUUAGCAGAAGAUGCAAAAU |
| | | UACAAUUAUCAAAAGAUACAUAUGAUGAUGAUUUAGAUAAUUUAUUAGCACAAAUAGGAGAUCAAUAUGCAGAUUUAUU |
| | | UUUAGCAGCAAAAAAUUUAUCAGAUGCAAUAUUAUUAUCAGAUAUAUUAAGAGUAAAUACAGAAAUAACAAAAGCACCA |
| | | UUAUCAGCAUCAAUGAUAAAAAGAUAUGAUGAACAUCAUCAGGACUUAACAUUAUUAAAAGCAUUAGUAAGACAACAAU |
| | | UACCAGAAAAAUAUAAAGAAAUAUUUUUUGAUCAAUCAAAAAAUGGAUAUGCAGGAUAUAUAGAUGGAGGAGCAUCACA |
| | | AGAAGAAUUUUAUAAAUUUAUAAAACCAAUAUUAGAAAAAAUGGAUGGAACAGAAGAAUUAUUAGUAAAAUUAAAUAGA |
| | | GAAGAUUUAUUAAGAAAACAAAGAACAUUUGAUAAUGGAUCAAUACCACAUCAAAUACAUUUAGGAGAAUUACAUGCAA |
| | | UAUUAAGAAGACAAGAAGAUUUUUAUCCAUUUUUAAAAGAUAAUAGAGAAAAAAUAGAAAAAAUAUUAACAUUUAGAAU |
| | | ACCAUAUUAUGUAGGACCAUUAGCAAGAGGAAAUUCAAGAUUUGCAUGGAUGACAAGAAAAUCAGAAGAAACAAUAACA |
| | | CCAUGGAAUUUUGAAGAAGUAGUAGAUAAAGGAGCAUCAGCACAAUCAUUUAUAGAAAGAAUGACAAAUUUUGAUAAAA |
| | | AUUUACCAAAUGAAAAAGUAUUACCAAAACAUUCAUUAUUAUAUGAAUAUUUUACAGUAUAUAAUGAAUUAACAAAAGU |
| | | AAAAUAUGUAACAGAAGGAAUGAGAAAACCAGCAUUUUUAUCAGGAGAACAAAAAAAAGCAAUAGUAGAUUUAUUAUUU |
| | | AAAACAAAUAGAAAAGUAACAGUAAAACAAUUAAAAGAAGAUUAUUUUAAAAAAAUAGAAUGUUUUGAUUCAGUAGAAA |
| | | UAUCAGGAGUAGAAGAUAGAUUUAAUGCAUCAUUAGGAACAUAUCAUGAUUUAUUAAAAAUAAUAAAAGAUAAAGAUUU |
| | | UUUAGAUAAUGAAGAAAAUGAAGAUAUAUUAGAAGAUAUAGUAUUAACAUUAACAUUAUUUGAAGAUAGAGAAAUGAUA |
| | | GAAGAAAGAUUAAAAACAUAUGCACAUUUAUUUGAUGAUAAAGUAAUGAAACAAUUAAAAAGAAGAAGAUAUACAGGAU |
| | | GGGGAAGAUUAUCAAGAAAAUUAAUAAAUGGAAUAAGAGAUAAACAAUCAGGAAAAACAAUAUUAGAUUUUUUAAAAUC |
| | | AGAUGGAUUUGCAAAUAGAAAUUUUAUGCAAUUAAUACAUGAUGAUUCAUUAACAUUUAAAGAAGAUAUACAAAAAGCA |
| | | CAAGUAUCAGGACAAGGAGAUUCAUUACAUGAACAUAUAGCAAAUUUAGCAGGAUCACCAGCAAUAAAAAAAGGAAUAU |
| | | UACAAACAGUAAAAGUAGUAGAUGAAUUAGUAAAAGUAAUGGGAAGACAUAAACCAGAAAAUAUAGUAAUAGAAAUGGC |
| | | AAGAGAAAAUCAAACAACACAAAAAGGACAAAAAAAUUCAAGAGAAAGAAUGAAAAGAAUAGAAGAAGGAAUAAAAGAA |
| | | UUAGGAUCACAAAUAUUAAAAGAACAUCCAGUAGAAAAUACACAAUUACAAAAUGAAAAAUUAUAUUUAUAUUAUUUAC |
| | | AAAAUGGAAGAGAUAUGUAUGUAGAUCAAGAAUUAGAUAUAAAUAGAUUAUCAGAUUAUGAUGUAGAUCAUAUAGUACC |
| | | ACAAUCAUUUUUAAAAGAUGAUUCAAUAGAUAAUAAAGUAUUAACAAGAUCAGAUAAAAAUAGAGGAAAAUCAGAUAAU |
| | | GUACCAUCAGAAGAAGUAGUAAAAAAAAUGAAAAAUUAUUGGAGACAAUUAUUAAAUGCAAAAUUAAUAACACAAAGAA |
| | | AAUUUGAUAAUUUAACAAAAGCAGAAAGAGGAGGAUUAUCAGAAUUAGAUAAAGCAGGAUUUAUAAAAAGACAAUUAGU |
| | | AGAAACAAGACAAAUAACAAAACAUGUAGCACAAAUAUUAGAUUCAAGAAUGAAUACAAAAUAUGAUGAAAAUGAUAAA |
| | | UUAAUAAGAGAAGUAAAAGUAAUAACAUUAAAAUCAAAAUUAGUAUCAGAUUUUAGAAAAGAUUUUCAAUUUUAUAAAG |
| | | UAAGAGAAAUAAAUAAUUAUCAUCAUGCACAUGAUGCAUAUUUAAAUGCAGUAGUAGGAACAGCAUUAAUAAAAAAAUA |
| | | UCCAAAAUUAGAAUCAGAAUUUGUAUAUGGAGAUUAUAAAGUAUAUGAUGUAAGAAAAAUGAUAGCAAAAUCAGAACAA |
| | | GAAAUAGGAAAAGCAACAGCAAAAUAUUUUUUUUAUUCAAAUAUAAUGAAUUUUUUUAAAACAGAAAUAACAUUAGCAA |
| | | AUGGAGAAAUAAGAAAAAGACCAUUAAUAGAAACAAAUGGAGAAACAGGAGAAAUAGUAUGGGAUAAAGGAAGAGAUUU |
| | | UGCAACAGUAAGAAAAGUAUUAUCAAUGCCACAAGUAAAUAUAGUAAAAAAAACAGAAGUACAAACAGGAGGAUUUUCA |
| | | AAAGAAUCAAUAUUACCAAAAAGAAAUUCAGAUAAAUUAAUAGCAAGAAAAAAAGAUUGGGAUCCAAAAAAAUAUGGAG |
| | | GAUUUGAUUCACCAACAGUAGCAUAUUCAGUAUUAGUAGUAGCAAAAGUAGAAAAAGGAAAAUCAAAAAAAUUAAAAUC |
| | | AGUAAAAGAAUUAUUAGGAAUAACAAUAAUGGAAAGAUCAUCAUUUGAAAAAAAUCCAAUAGAUUUUUUAGAAGCAAAA |
| | | GGAUAUAAAGAAGUAAAAAAAGAUUUAAUAAUAAAAUUACCAAAAUAUUCAUUAUUUGAAUUAGAAAAUGGAAGAAAAA |
| | | GAAUGUUAGCAUCAGCAGGAGAAUUACAAAAAGGAAAUGAAUUAGCAUUACCAUCAAAAUAUGUAAAUUUUUUAUAUUU |
| | | AGCAUCACAUUAUGAAAAAUUAAAAGGAUCACCAGAAGAUAAUGAACAAAAACAAUUAUUUGUAGAACAACAUAAACAU |
| | | UAUUUAGAUGAAAUAAUAGAACAAAUAUCAGAAUUUUCAAAAAGAGUAAUAUUAGCAGAUGCAAAUUUAGAUAAAGUAU |
| | | UAUCAGCAUAUAAUAAACAUAGAGAUAAACCAAUAAGAGAACAAGCAGAAAAUAUAAUACAUUUAUUUACAUUAACAAA |
| | | UUUAGGAGCACCAGCAGCAUUUAAAUAUUUUGAUACAACAAUAGAUAGAAAAAGAUAUACAUCAACAAAAGAAGUAUUA |
| | | GAUGCAACAUUAAUACAUCAAUCAAUAACAGGAUUAUAUGAAACAAGAAUAGAUUUAUCACAAUUAGGAGGAGAUGGAG |
| | | GAGGAUCACCAAAAAAAAAAAGAAAAGUAUAG |
| 14 | I-pair enriched | AUGGAUAAAAAGUACAGCAUCGGAUUAGAUAUAGGAACAAAUUCAGUUGGCUGGGCUGUGAUAACAGAUGAAUAUAAAG |
| | Cas9 ORF | UUCCCUCAAAAAAAUUUAAAGUAUUAGGAAAUACAGAUAGACAUAGCAUCAAAAAAAAUCUCAUAGGUGCACUGUUAUU |
| | | UGAUUCAGGUGAGACAGCAGAAGCCACAAGAUUAAAAAGAACAGCCCGCAGAAGAUAUACAAGAAGAAAAAAUAGAAUA |
| | | UGUUAUUUACAGGAGAUAUUUUCAAAUGAAAUGGCAAAAGUAGAUGAUUCAUUUUUUCAUAGAUUAGAAGAAUCAUUCC |
| | | UGGUAGAAGAAGAUAAAAAACAUGAAAGACAUCCAAUAUUUGGAAAUAUAGUAGAUGAAGUCGCAUAUCAUGAAAAGUA |
| | | CCCCACCAUAUAUCAUCUGCGGAAAAAAUUAGUAGAUUCGACUGAUAAAGCAGAUCUGCGGUUAAUAUAUUUAGCACUGG |
| | | CACAUAUGAUAAAAUUUAGAGGACAUUUCCUGAUAGAAGGAGAUUUAAAUCCUGACAAUUCAGAUGUAGAUAAAUUAUU |
| | | UAUACAAUUAGUACAAACCUACAAUCAAUUAUUUGAAGAAAAUCCAAUAAAUGCAUCAGGAGUAGAUGCAAAAGCAAUA |
| | | CUCAGCGCCCGCCUCAGCAAAUCAAGAAGAUUAGAAAAUCUCAUAGCACAACUUCCAGGUGAGAAAAAAAAUGGGUUAUU |
| | | UGGAAAUCUCAUAGCACUCAGCUUAGGAUUAACUCCCAAUUUUAAAUCAAAUUUUGAUUUAGCAGAAGAUGCAAAAUUA |
| | | CAACUCAGCAAAGAUACCUACGAUGAUGAUUUAGAUAAUCUCUUAGCACAAAUAGGAGAUCAAUAUGCAGAUUUAUUCCU |
| | | GGCUGCCAAAAAUCUCAGCGAUGCAAUAUUACUCAGCGAUAUACUGCGGGUAAAUACAGAGAUAACAAAAGCACCACUCA |
| | | GCGCAUCAAUGAUAAAAAGAUAUGAUGAACAUCAUCAAGAUUUAACAUUAUUAAAAGCACUGGUAAGACAACAACUUCC |
| | | AGAGAAGUACAAAGAAAUAUUUUUUGAUCAGAGCAAAAAUGGGUAUGCCGGGUAUAUAGAUGGUGGUGCCUCACAGGAG |
| | | GAAUUUUAUAAAUUUAUAAAACCAAUAUUAGAAAAAAUGGAUGGAACAGAGGAGCUGUUAGUAAAAUUAAAUAGGGAGG |
| | | AUUUACUGCGGAAACAAAGAACAUUUGAUAAUGGGAGCAUCCCCCAUCAAAUACAUUUAGGUGAGCUGCAUGCAAUACUG |
| | | CGGAGACAGGAGGAUUUUUAUCCAUUCCUGAAAGAUAAUAGGGAGAAAAUAGAAAAAAUAUUAACAUUUAGAAUCCCCU |
| | | AUUAUGUUGGCCCAUUAGCCCGCGGAAAUUCAAGAUUUGCAUGGAUGACAAGAAAAUCAGAAGAAACAAUAACUCCCUGG |
| | | AAUUUUGAAGAAGUCGUAGAUAAGGGUGCCUCAGCACAGAGCUUUAUAGAAAGAAUGACAAAUUUUGAUAAAAAUCUUC |
| | | CAAAUGAAAAAGUACUUCCAAAACAUUCAUUAUUAUAUGAAUAUUUUACAGUAUAUAAUGAGCUGACAAAAGUAAAGUA |
| | | CGUAACAGAGGGAAUGAGAAAACCAGCAUUCCUCAGCGGUGAGCAAAAAAAAGCAAUAGUAGAUUUAUUAUUUAAAACA |
| | | AAUAGAAAAGUAACAGUAAAACAAUUAAAAGAAGAUUAUUUUAAAAAAAUAGAAUGUUUUGAUUCAGUAGAAAUAUCAG |
| | | GAGUAGAAGAUAGAUUUAAUGCAUCAUUAGGAACCUACCAUGAUUUAUUAAAAAUAAUAAAAGAUAAAGAUUUCCUGGA |
| | | UAAUGAAGAAAAUGAAGAUAUAUUAGAAGAUAUAGUAUUAACAUUAACAUUAUUUGAAGAUAGGGAGAUGAUAGAAGAA |
| | | AGAUUAAAAACCUACGCACAUUUAUUUGAUGAUAAAGUAAUGAAACAAUUAAAAAGAAGAAGAUAUACAGGAUGGGGAA |
| | | GACUCAGCAGAAAAUUAAUAAAUGGGAUACGAGACAAACAGAGCGGAAAAACAAUAUUAGAUUUCCUGAAAUCAGAUGG |
| | | AUUUGCAAAUAGAAAUUUUAUGCAAUUAAUACAUGAUGAUUCAUUAACAUUUAAAGAAGAUAUACAAAAAGCACAGGUC |
| | | AGCGGACAGGGCGAUUCAUUACAUGAACAUAUAGCAAAUCUCGCCGGGUCACCAGCAAUAAAAAAGGGGAUAUUACAAAC |
| | | AGUAAAAGUAGUAGAUGAGCUGGUAAAAGUAAUGGGAAGACAUAAACCAGAGAAUAUAGUAAUAGAAAUGGCCAGGGAG |
| | | AAUCAAACAACUCAAAAGGGGCAAAAAAAUUCAAGGGAGAGAAUGAAAAGAAUAGAAGAAGGAAUAAAAGAGCUGGGAU |
| | | CACAAAUAUUAAAAGAACAUCCAGUAGAAAAUACUCAAUUACAAAAUGAAAAAUUAUAUUUAUAUUAUUUACAAAAUGG |
| | | GCGAGACAUGUAUGUAGAUCAGGAGCUGGAUAUAAAUAGACUCAGCGAUUAUGAUGUAGAUCAUAUAGUUCCCCAGAGC |
| | | UUCCUGAAAGAUGAUAGCAUCGAUAAUAAAGUAUUAACAAGAUCAGAUAAAAAUAGAGGAAAAUCAGAUAAUGUUCCCU |
| | | CAGAAGAAGUCGUAAAAAAAAUGAAAAAUUAUUGGAGACAAUUAUUAAAUGCAAAAUUAAUAACUCAAAGAAAAUUUGA |
| | | UAAUCUCACAAAAGCAGAAAGAGGUGGCCUCAGCGAGCUGGAUAAAGCCGGGUUUAUAAAAAGACAAUUAGUAGAAACA |
| | | AGACAAAUAACAAAACAUGUAGCACAAAUAUUAGAUUCAAGAAUGAAUACAAAGUACGAUGAAAAUGAUAAAUUAAUAA |
| | | GGGAAGUCAAAGUAAUAACAUUAAAAUCAAAAUUAGUCAGCGAUUUUAGAAAAGAUUUUCAAUUUUAUAAAGUAAGGGA |
| | | GAUAAAUAAUUAUCAUCAUGCACAUGAUGCAUAUUUAAAUGCUGUGGUUGGCACAGCACUGAUAAAAAAGUACCCAAAA |
| | | UUAGAAUCAGAAUUUGUAUAUGGAGAUUAUAAAGUAUAUGAUGUAAGAAAAAUGAUAGCAAAAUCAGAACAGGAGAUAG |
| | | GAAAAGCAACAGCAAAGUACUUUUUUUAUUCAAAUAUAAUGAAUUUUUUUAAAACAGAGAUAACAUUAGCAAAUGGUGA |
| | | GAUAAGAAAAAGACCAUUAAUAGAAACAAAUGGUGAGACAGGUGAGAUAGUAUGGGAUAAGGGGCGAGACUUUGCAACA |
| | | GUAAGAAAAGUACUCAGCAUGCCACAGGUGAAUAUAGUAAAAAAAACAGAAGUCCAAACAGGUGGCUUUUCAAAAGAAA |
| | | GCAUCCUUCCAAAAAGAAAUUCAGAUAAAUUAAUAGCCCGCAAAAAAGAUUGGGAUCCAAAAAAGUACGGUGGCUUUGA |
| | | UUCACCCACCGUAGCAUAUUCAGUAUUAGUAGUAGCAAAAGUAGAAAAGGGGAAAUCAAAAAAAUUAAAAUCAGUAAAA |
| | | GAGCUGUUAGGAAUAACAAUAAUGGAAAGAUCAUCAUUUGAAAAAAAUCCAAUAGAUUUCCUGGAAGCCAAGGGGUAUA |
| | | AAGAAGUCAAAAAAGAUUUAAUAAUAAAACUUCCAAAGUACUCAUUAUUUGAGCUGGAAAAUGGGAGAAAAAGAAUGUU |
| | | AGCAUCAGCCGGUGAGCUGCAAAAGGGGAAUGAGCUGGCACUUCCCUCAAAGUACGUAAAUUUCCUGUAUUUAGCAUCAC |
| | | AUUAUGAAAAAUUAAAGGGGUCACCAGAGGAUAAUGAACAAAAACAAUUAUUUGUAGAACAACAUAAACAUUAUUUAGA |
| | | UGAAAUAAUAGAACAAAUAUCAGAAUUUUCAAAAAGAGUAAUAUUAGCAGAUGCAAAUCUCGAUAAAGUACUCAGCGCA |
| | | UAUAAUAAACAUCGAGACAAACCAAUAAGGGAGCAGGCCGAAAAUAUAAUACAUUUAUUUACAUUAACAAAUCUCGGUG |
| | | CCCCAGCUGCCUUUAAGUACUUUGAUACAACAAUAGAUAGAAAAAGAUAUACAUCGACUAAAGAAGUCUUAGAUGCAACA |
| | | UUAAUACAUCAGAGCAUCACAGGAUUAUAUGAAACAAGAAUAGAUCUCAGCCAAUUAGGUGGCGAUGGUGGUGGCUCACC |
| | | AAAAAAAAAAAGAAAAGUAUAG |
| 15 | Cas9 mRNA | GGGAAGCUCAGAAUAAACGCUCAACUUUGGCCGGAUCUGCCACCAUGGACAAGAAGUACAGCAUCGGCCUGGACAUCGGC |
| | transcript | ACGAACAGCGUGGGCUGGGCCGUGAUCACGGACGAGUACAAGGUGCCCAGCAAGAAGUUCAAGGUGCUGGGCAACACGGA |
| | comprising SEQ | CCGGCACAGCAUCAAGAAGAACCUGAUCGGCGCCCUGCUGUUCGACAGCGGCGAGACGGCCGAGGCCACGCGGCUGAAGC |
| | 5 | GGACGGCCCGGCGGCGGUACACGCGGCGGAAGAACCGGAUCUGCUACCUGCAGGAGAUCUUCAGCAACGAGAUGGCCAAG |
| | | GUGGACGACAGCUUCUUCCACCGGCUGGAGGAGAGCUUCCUGGUGGAGGAGGACAAGAAGCACGAGCGGCACCCCAUCUU |
| | | CGGCAACAUCGUGGACGAGGUGGCCUACCACGAGAAGUACCCCACGAUCUACCACCUGCGGAAGAAGCUGGUGGACAGCA |
| | | CGGACAAGGCCGACCUGCGGCUGAUCUACCUGGCCCUGGCCCACAUGAUCAAGUUCCGGGGCCACUUCCUGAUCGAGGGCG |
| | | ACCUGAACCCCGACAACAGCGACGUGGACAAGCUGUUCAUCCAGCUGGUGCAGACGUACAACCAGCUGUUCGAGGAGAAC |
| | | CCCAUCAACGCCAGCGGCGUGGACGCCAAGGCCAUCCUGAGCGCCCGGCUGAGCAAGAGCCGGCGGCUGGAGAACCUGAUC |
| | | GCCCAGCUGCCCGGCGAGAAGAAGAACGGCCUGUUCGGCAACCUGAUCGCCCUGAGCCUGGGCCUGACGCCCAACUUCAAG |
| | | AGCAACUUCGACCUGGCCGAGGACGCCAAGCUGCAGCUGAGCAAGGACACGUACGACGACGACCUGGACAACCUGCUGGC |
| | | CCAGAUCGGCGACCAGUACGCCGACCUGUUCCUGGCCGCCAAGAACCUGAGCGACGCCAUCCUGCUGAGCGACAUCCUGCG |
| | | GGUGAACACGGAGAUCACGAAGGCCCCCCUGAGCGCCAGCAUGAUCAAGCGGUACGACGAGCACCACCAGGACCUGACGC |
| | | UGCUGAAGGCCCUGGUGCGGCAGCAGCUGCCCGAGAAGUACAAGGAGAUCUUCUUCGACCAGAGCAAGAACGGCUACGCC |
| | | GGCUACAUCGACGGCGGCGCCAGCCAGGAGGAGUUCUACAAGUUCAUCAAGCCCAUCCUGGAGAAGAUGGACGGCACGGA |
| | | GGAGCUGCUGGUGAAGCUGAACCGGGAGGACCUGCUGCGGAAGCAGCGGACGUUCGACAACGGCAGCAUCCCCCACCAGA |
| | | UCCACCUGGGCGAGCUGCACGCCAUCCUGCGGCGGCAGGAGGACUUCUACCCCUUCCUGAAGGACAACCGGGAGAAGAUC |
| | | GAGAAGAUCCUGACGUUCCGGAUCCCCUACUACGUGGGCCCCCUGGCCCGGGGCAACAGCCGGUUCGCCUGGAUGACGCGG |
| | | AAGAGCGAGGAGACGAUCACGCCCUGGAACUUCGAGGAGGUGGUGGACAAGGGCGCCAGCGCCCAGAGCUUCAUCGAGCG |
| | | GAUGACGAACUUCGACAAGAACCUGCCCAACGAGAAGGUGCUGCCCAAGCACAGCCUGCUGUACGAGUACUUCACGGUGU |
| | | ACAACGAGCUGACGAAGGUGAAGUACGUGACGGAGGGCAUGCGGAAGCCCGCCUUCCUGAGCGGCGAGCAGAAGAAGGCC |
| | | AUCGUGGACCUGCUGUUCAAGACGAACCGGAAGGUGACGGUGAAGCAGCUGAAGGAGGACUACUUCAAGAAGAUCGAGUG |
| | | CUUCGACAGCGUGGAGAUCAGCGGCGUGGAGGACCGGUUCAACGCCAGCCUGGGCACGUACCACGACCUGCUGAAGAUCA |
| | | UCAAGGACAAGGACUUCCUGGACAACGAGGAGAACGAGGACAUCCUGGAGGACAUCGUGCUGACGCUGACGCUGUUCGAG |
| | | GACCGGGAGAUGAUCGAGGAGCGGCUGAAGACGUACGCCCACCUGUUCGACGACAAGGUGAUGAAGCAGCUGAAGCGGCG |
| | | GCGGUACACGGGCUGGGGCCGGCUGAGCCGGAAGCUGAUCAACGGCAUCCGGGACAAGCAGAGCGGCAAGACGAUCCUGG |
| | | ACUUCCUGAAGAGCGACGGCUUCGCCAACCGGAACUUCAUGCAGCUGAUCCACGACGACAGCCUGACGUUCAAGGAGGAC |
| | | AUCCAGAAGGCCCAGGUGAGCGGCCAGGGCGACAGCCUGCACGAGCACAUCGCCAACCUGGCCGGCAGCCCCGCCAUCAAG |
| | | AAGGGCAUCCUGCAGACGGUGAAGGUGGUGGACGAGCUGGUGAAGGUGAUGGGCCGGCACAAGCCCGAGAACAUCGUGAU |
| | | CGAGAUGGCCCGGGAGAACCAGACGACGCAGAAGGGCCAGAAGAACAGCCGGGAGCGGAUGAAGCGGAUCGAGGAGGGCA |
| | | UCAAGGAGCUGGGCAGCCAGAUCCUGAAGGAGCACCCCGUGGAGAACACGCAGCUGCAGAACGAGAAGCUGUACCUGUAC |
| | | UACCUGCAGAACGGCCGGGACAUGUACGUGGACCAGGAGCUGGACAUCAACCGGCUGAGCGACUACGACGUGGACCACAU |
| | | CGUGCCCCAGAGCUUCCUGAAGGACGACAGCAUCGACAACAAGGUGCUGACGCGGAGCGACAAGAACCGGGGCAAGAGCG |
| | | ACAACGUGCCCAGCGAGGAGGUGGUGAAGAAGAUGAAGAACUACUGGCGGCAGCUGCUGAACGCCAAGCUGAUCACGCAG |
| | | CGGAAGUUCGACAACCUGACGAAGGCCGAGCGGGGCGGCCUGAGCGAGCUGGACAAGGCCGGCUUCAUCAAGCGGCAGCU |
| | | GGUGGAGACGCGGCAGAUCACGAAGCACGUGGCCCAGAUCCUGGACAGCCGGAUGAACACGAAGUACGACGAGAACGACA |
| | | AGCUGAUCCGGGAGGUGAAGGUGAUCACGCUGAAGAGCAAGCUGGUGAGCGACUUCCGGAAGGACUUCCAGUUCUACAAG |
| | | GUGCGGGAGAUCAACAACUACCACCACGCCCACGACGCCUACCUGAACGCCGUGGUGGGCACGGCCCUGAUCAAGAAGUAC |
| | | CCCAAGCUGGAGAGCGAGUUCGUGUACGGCGACUACAAGGUGUACGACGUGCGGAAGAUGAUCGCCAAGAGCGAGCAGGA |
| | | GAUCGGCAAGGCCACGGCCAAGUACUUCUUCUACAGCAACAUCAUGAACUUCUUCAAGACGGAGAUCACGCUGGCCAACG |
| | | GCGAGAUCCGGAAGCGGCCCCUGAUCGAGACGAACGGCGAGACGGGCGAGAUCGUGUGGGACAAGGGCCGGGACUUCGCC |
| | | ACGGUGCGGAAGGUGCUGAGCAUGCCCCAGGUGAACAUCGUGAAGAAGACGGAGGUGCAGACGGGCGGCUUCAGCAAGGA |
| | | GAGCAUCCUGCCCAAGCGGAACAGCGACAAGCUGAUCGCCCGGAAGAAGGACUGGGACCCCAAGAAGUACGGCGGCUUCG |
| | | ACAGCCCCACGGUGGCCUACAGCGUGCUGGUGGUGGCCAAGGUGGAGAAGGGCAAGAGCAAGAAGCUGAAGAGCGUGAAG |
| | | GAGCUGCUGGGCAUCACGAUCAUGGAGCGGAGCAGCUUCGAGAAGAACCCCAUCGACUUCCUGGAGGCCAAGGGCUACAA |
| | | GGAGGUGAAGAAGGACCUGAUCAUCAAGCUGCCCAAGUACAGCCUGUUCGAGCUGGAGAACGGCCGGAAGCGGAUGCUGG |
| | | CCAGCGCCGGCGAGCUGCAGAAGGGCAACGAGCUGGCCCUGCCCAGCAAGUACGUGAACUUCCUGUACCUGGCCAGCCACU |
| | | ACGAGAAGCUGAAGGGCAGCCCCGAGGACAACGAGCAGAAGCAGCUGUUCGUGGAGCAGCACAAGCACUACCUGGACGAG |
| | | AUCAUCGAGCAGAUCAGCGAGUUCAGCAAGCGGGUGAUCCUGGCCGACGCCAACCUGGACAAGGUGCUGAGCGCCUACAA |
| | | CAAGCACCGGGACAAGCCCAUCCGGGAGCAGGCCGAGAACAUCAUCCACCUGUUCACGCUGACGAACCUGGGCGCCCCCGC |
| | | CGCCUUCAAGUACUUCGACACGACGAUCGACCGGAAGCGGUACACGAGCACGAAGGAGGUGCUGGACGCCACGCUGAUCC |
| | | ACCAGAGCAUCACGGGCCUGUACGAGACGCGGAUCGACCUGAGCCAGCUGGGCGGCGACGGCGGCGGCAGCCCCAAGAAG |
| | | AAGCGGAAGGUGUAGCUAGCACCAGCCUCAAGAACACCCGAAUGGAGUCUCUAAGCUACAUAAUACCAACUUACACUUUA |
| | | CAAAAUGUUGUCCCCCAAAAUGUAGCCAUUCGUAUCUGCUCCUAAUAAAAAGAAAGUUUCUUCACAUUCUCUCGAGAAAA |
| | | AAAAAAAAAAAAAAAAAAAAAAAAAAAAAAAAAAAAAAAAAAAAAAAAAAAAAAAAAAAAAAAAAAAAAAAAAAAAAA |
| | | AAAAAAAAAAAAAUCUAG |
| 16 | Cas9 mRNA | GGGAAGCUCAGAAUAAACGCUCAACUUUGGCCGGAUCUGCCACCAUGGACAAGAAGUACAGCAUCGGCCUCGACAUCGGC |
| | transcript | ACCAACAGCGUUGGCUGGGCUGUGAUCACCGACGAGUACAAGGUUCCCUCAAAGAAGUUCAAGGUCCUCGGCAACACCGA |
| | comprising SEQ | CCGCCACAGCAUCAAGAAGAAUCUCAUCGGUGCACUGCUCUUCGACAGCGGUGAGACCGCGGAAGCCACCCGCCUCAAGCG |
| | 7 | GACCGCCCGCCGCCGCUACACCCGCCGCAAGAACCGCAUCUGCUACCUCCAGGAGAUCUUCAGCAACGAGAUGGCCAAGGU |
| | | CGACGACAGCUUCUUCCACCGCCUCGAGGAGAGCUUCCUGGUCGAGGAGGACAAGAAGCACGAGCGCCACCCCAUCUUCGG |
| | | CAACAUCGUCGACGAAGUCGCCUACCACGAGAAGUACCCCACCAUCUACCACCUGCGGAAGAAGCUCGUCGACUCGACUGA |
| | | CAAGGCCGACCUGCGGCUCAUCUACCUCGCACUGGCCCACAUGAUAAAGUUCCGCGGCCACUUCCUGAUCGAGGGCGACCU |
| | | CAACCCUGACAACAGCGACGUCGACAAGCUCUUCAUCCAGCUCGUCCAGACCUACAACCAGCUCUUCGAGGAGAACCCCAU |
| | | CAACGCCAGCGGCGUCGACGCCAAGGCCAUCCUCAGCGCCCGCCUCAGCAAGAGCCGCCGCCUCGAGAAUCUCAUCGCCCA |
| | | GCUUCCAGGUGAGAAGAAGAAUGGGCUCUUCGGCAAUCUCAUCGCACUCAGCCUCGGCCUCACUCCCAACUUCAAGAGCA |
| | | ACUUCGACCUCGCGGAGGACGCCAAGCUCCAGCUCAGCAAGGACACCUACGACGACGACCUCGACAAUCUCCUCGCCCAGA |
| | | UCGGCGACCAGUACGCCGACCUCUUCCUGGCUGCCAAGAAUCUCAGCGACGCCAUCCUCCUCAGCGACAUCCUGCGGGUCA |
| | | ACACAGAGAUCACCAAGGCCCCCCUCAGCGCCAGCAUGAUAAAGCGCUACGACGAGCACCACCAGGACCUCACCCUCCUCA |
| | | AGGCACUGGUCCGCCAGCAGCUUCCAGAGAAGUACAAGGAGAUCUUCUUCGACCAGAGCAAGAAUGGGUACGCCGGGUAC |
| | | AUCGACGGUGGUGCCAGCCAGGAGGAGUUCUACAAGUUCAUCAAGCCCAUCCUCGAGAAGAUGGACGGCACAGAGGAGCU |
| | | GCUCGUCAAGCUCAACAGGGAGGACCUCCUGCGGAAGCAGCGGACCUUCGACAAUGGGAGCAUCCCCCACCAGAUCCACCU |
| | | CGGUGAGCUGCACGCCAUCCUGCGGCGCCAGGAGGACUUCUACCCCUUCCUGAAGGACAACAGGGAGAAGAUCGAGAAGA |
| | | UCCUCACCUUCCGCAUCCCCUACUACGUUGGCCCCCUCGCCCGCGGCAACAGCCGCUUCGCCUGGAUGACCCGCAAGAGCG |
| | | AGGAGACCAUCACUCCCUGGAACUUCGAGGAAGUCGUCGACAAGGGUGCCAGCGCCCAGAGCUUCAUCGAGCGCAUGACC |
| | | AACUUCGACAAGAAUCUUCCAAACGAGAAGGUCCUUCCAAAGCACAGCCUCCUCUACGAGUACUUCACCGUCUACAACGA |
| | | GCUGACCAAGGUCAAGUACGUCACAGAGGGCAUGCGCAAGCCAGCCUUCCUCAGCGGUGAGCAGAAGAAGGCCAUCGUCG |
| | | ACCUCCUCUUCAAGACCAACCGCAAGGUCACCGUCAAGCAGCUCAAGGAGGACUACUUCAAGAAGAUCGAGUGCUUCGAC |
| | | AGCGUCGAGAUCAGCGGCGUCGAGGACCGCUUCAACGCCAGCCUCGGCACCUACCACGACCUCCUCAAGAUCAUCAAGGAC |
| | | AAGGACUUCCUGGACAACGAGGAGAACGAGGACAUCCUCGAGGACAUCGUCCUCACCCUCACCCUCUUCGAGGACAGGGA |
| | | GAUGAUAGAGGAGCGCCUCAAGACCUACGCCCACCUCUUCGACGACAAGGUCAUGAAGCAGCUCAAGCGCCGCCGCUACAC |
| | | CGGCUGGGGCCGCCUCAGCCGCAAGCUCAUCAAUGGGAUCCGAGACAAGCAGAGCGGCAAGACCAUCCUCGACUUCCUGA |
| | | AGAGCGACGGCUUCGCCAACCGCAACUUCAUGCAGCUCAUCCACGACGACAGCCUCACCUUCAAGGAGGACAUCCAGAAGG |
| | | CCCAGGUCAGCGGCCAGGGCGACAGCCUCCACGAGCACAUCGCCAAUCUCGCCGGGAGCCCAGCCAUCAAGAAGGGGAUCC |
| | | UCCAGACCGUCAAGGUCGUCGACGAGCUGGUCAAGGUCAUGGGCCGCCACAAGCCAGAGAACAUCGUCAUCGAGAUGGCC |
| | | AGGGAGAACCAGACCACUCAAAAGGGGCAGAAGAACAGCAGGGAGCGCAUGAAGCGCAUCGAGGAGGGCAUCAAGGAGCU |
| | | GGGCAGCCAGAUCCUCAAGGAGCACCCCGUCGAGAACACUCAACUCCAGAACGAGAAGCUCUACCUCUACUACCUCCAGAA |
| | | UGGGCGAGACAUGUACGUCGACCAGGAGCUGGACAUCAACCGCCUCAGCGACUACGACGUCGACCACAUCGUUCCCCAGA |
| | | GCUUCCUGAAGGACGACAGCAUCGACAACAAGGUCCUCACCCGAAGCGACAAGAACCGCGGCAAGAGCGACAACGUUCCC |
| | | UCAGAGGAAGUCGUCAAGAAGAUGAAGAACUACUGGCGCCAGCUCCUCAACGCCAAGCUCAUCACUCAACGCAAGUUCGA |
| | | CAAUCUCACCAAGGCGGAGCGCGGUGGCCUCAGCGAGCUGGACAAGGCCGGGUUCAUCAAGCGCCAGCUCGUCGAGACCC |
| | | GCCAGAUCACCAAGCACGUCGCCCAGAUCCUCGACAGCCGCAUGAACACCAAGUACGACGAGAACGACAAGCUCAUCAGGG |
| | | AAGUCAAGGUCAUCACCCUCAAGAGCAAGCUCGUCAGCGACUUCCGCAAGGACUUCCAGUUCUACAAGGUCAGGGAGAUC |
| | | AACAACUACCACCACGCCCACGACGCCUACCUCAACGCUGUGGUUGGCACCGCACUGAUCAAGAAGUACCCCAAGCUCGAG |
| | | AGCGAGUUCGUCUACGGCGACUACAAGGUCUACGACGUCCGCAAGAUGAUAGCCAAGAGCGAGCAGGAGAUCGGCAAGGC |
| | | CACCGCCAAGUACUUCUUCUACAGCAACAUCAUGAACUUCUUCAAGACAGAGAUCACCCUCGCCAAUGGUGAGAUCCGCA |
| | | AGCGCCCCCUCAUCGAGACCAAUGGUGAGACCGGUGAGAUCGUCUGGGACAAGGGGCGAGACUUCGCCACCGUCCGCAAG |
| | | GUCCUCAGCAUGCCCCAGGUGAACAUCGUCAAGAAGACAGAAGUCCAGACCGGUGGCUUCAGCAAGGAGAGCAUCCUUCC |
| | | AAAGCGCAACAGCGACAAGCUCAUCGCCCGCAAGAAGGACUGGGACCCCAAGAAGUACGGUGGCUUCGACAGCCCCACCG |
| | | UCGCCUACAGCGUCCUCGUCGUCGCCAAGGUCGAGAAGGGGAAGAGCAAGAAGCUCAAGAGCGUCAAGGAGCUGCUCGGC |
| | | AUCACCAUCAUGGAGCGAAGCAGCUUCGAGAAGAACCCCAUCGACUUCCUGGAAGCCAAGGGGUACAAGGAAGUCAAGAA |
| | | GGACCUCAUCAUCAAGCUUCCAAAGUACAGCCUCUUCGAGCUGGAGAAUGGGCGCAAGCGCAUGCUCGCCAGCGCCGGUG |
| | | AGCUGCAGAAGGGGAACGAGCUGGCACUUCCCUCAAAGUACGUCAACUUCCUGUACCUCGCCAGCCACUACGAGAAGCUC |
| | | AAGGGGAGCCCAGAGGACAACGAGCAGAAGCAGCUCUUCGUCGAGCAGCACAAGCACUACCUCGACGAGAUCAUCGAGCA |
| | | GAUCAGCGAGUUCAGCAAGCGCGUCAUCCUCGCCGACGCCAAUCUCGACAAGGUCCUCAGCGCCUACAACAAGCACCGAGA |
| | | CAAGCCCAUCAGGGAGCAGGCCGAGAACAUCAUCCACCUCUUCACCCUCACCAAUCUCGGUGCCCCAGCUGCCUUCAAGUA |
| | | CUUCGACACCACCAUCGACCGCAAGCGCUACACCUCGACUAAGGAAGUCCUCGACGCCACCCUCAUCCACCAGAGCAUCAC |
| | | CGGCCUCUACGAGACCCGCAUCGACCUCAGCCAGCUCGGUGGCGACGGUGGUGGCAGCCCCAAGAAGAAGCGCAAGGUCU |
| | | AGCUAGCACCAGCCUCAAGAACACCCGAAUGGAGUCUCUAAGCUACAUAAUACCAACUUACACUUUACAAAAUGUUGUCC |
| | | CCCAAAAUGUAGCCAUUCGUAUCUGCUCCUAAUAAAAAGAAAGUUUCUUCACAUUCUCUCGAGAAAAAAAAAAAAAAAAA |
| | | AAAAAAAAAAAAAAAAAAAAAAAAAAAAAAAAAAAAAAAAAAAAAAAAAAAAAAAAAAAAAAAAAAAAAAAAAAAAAA |
| | | UCUAG |
| 17 | Cas9 mRNA | GGGAAGCUCAGAAUAAACGCUCAACUUUGGCCGGAUCUGCCACCAUGGACAAGAAGUACAGCAUCGGCCUCGACAUCGGC |
| | transcript | ACCAACAGCGUUGGCUGGGCUGUGAUCACCGACGAGUACAAGGUUCCCUCAAAGAAGUUCAAGGUCCUCGGCAACACCGA |
| | comprising SEQ | CCGCCACAGCAUCAAGAAGAAUCUCAUCGGUGCACUGCUCUUCGACAGCGGUGAGACCGCGGAAGCCACCCGCCUCAAGCG |
| | 9 | CACCGCCCGCCGCCGCUACACCCGCCGCAAGAACCGCAUCUGCUACCUCCAGGAGAUCUUCAGCAACGAGAUGGCCAAGGU |
| | | CGACGACAGCUUCUUCCACCGCCUCGAGGAGAGCUUCCUGGUCGAGGAGGACAAGAAGCACGAGCGCCACCCCAUCUUCGG |
| | | CAACAUCGUCGACGAAGUCGCCUACCACGAGAAGUACCCCACCAUCUACCACCUGCGGAAGAAGCUCGUCGACUCGACUGA |
| | | CAAGGCCGACCUGCGGCUCAUCUACCUCGCACUGGCCCACAUGAUAAAGUUCCGCGGCCACUUCCUGAUCGAGGGCGACCU |
| | | CAACCCUGACAACAGCGACGUCGACAAGCUCUUCAUCCAGCUCGUCCAGACCUACAACCAGCUCUUCGAGGAGAACCCCAU |
| | | CAACGCCAGCGGCGUCGACGCCAAGGCCAUCCUCAGCGCCCGCCUCAGCAAGAGCCGCCGCCUCGAGAAUCUCAUCGCCCA |
| | | GCUUCCAGGUGAGAAGAAGAAUGGGCUCUUCGGCAAUCUCAUCGCACUCAGCCUCGGCCUCACUCCCAACUUCAAGAGCA |
| | | ACUUCGACCUCGCGGAGGACGCCAAGCUCCAGCUCAGCAAGGACACCUACGACGACGACCUCGACAAUCUCCUCGCCCAGA |
| | | UCGGCGACCAGUACGCCGACCUCUUCCUGGCUGCCAAGAAUCUCAGCGACGCCAUCCUCCUCAGCGACAUCCUGCGGGUCA |
| | | ACACAGAGAUCACCAAGGCCCCCCUCAGCGCCAGCAUGAUAAAGCGCUACGACGAGCACCACCAGGACCUCACCCUCCUCA |
| | | AGGCACUGGUCCGCCAGCAGCUUCCAGAGAAGUACAAGGAGAUCUUCUUCGACCAGAGCAAGAAUGGGUACGCCGGGUAC |
| | | AUCGACGGUGGUGCCAGCCAGGAGGAGUUCUACAAGUUCAUCAAGCCCAUCCUCGAGAAGAUGGACGGCACAGAGGAGCU |
| | | GCUCGUCAAGCUCAACAGGGAGGACCUCCUGCGGAAGCAGCGCACCUUCGACAAUGGGAGCAUCCCCCACCAGAUCCACCU |
| | | CGGUGAGCUGCACGCCAUCCUGCGGCGCCAGGAGGACUUCUACCCCUUCCUGAAGGACAACAGGGAGAAGAUCGAGAAGA |
| | | UCCUCACCUUCCGCAUCCCCUACUACGUUGGCCCCCUCGCCCGCGGCAACAGCCGCUUCGCCUGGAUGACCCGCAAGAGCG |
| | | AGGAGACCAUCACUCCCUGGAACUUCGAGGAAGUCGUCGACAAGGGUGCCAGCGCCCAGAGCUUCAUCGAGCGCAUGACC |
| | | AACUUCGACAAGAAUCUUCCAAACGAGAAGGUCCUUCCAAAGCACAGCCUCCUCUACGAGUACUUCACCGUCUACAACGA |
| | | GCUGACCAAGGUCAAGUACGUCACAGAGGGCAUGCGCAAGCCAGCCUUCCUCAGCGGUGAGCAGAAGAAGGCCAUCGUCG |
| | | ACCUCCUCUUCAAGACCAACCGCAAGGUCACCGUCAAGCAGCUCAAGGAGGACUACUUCAAGAAGAUCGAGUGCUUCGAC |
| | | AGCGUCGAGAUCAGCGGCGUCGAGGACCGCUUCAACGCCAGCCUCGGCACCUACCACGACCUCCUCAAGAUCAUCAAGGAC |
| | | AAGGACUUCCUGGACAACGAGGAGAACGAGGACAUCCUCGAGGACAUCGUCCUCACCCUCACCCUCUUCGAGGACAGGGA |
| | | GAUGAUAGAGGAGCGCCUCAAGACCUACGCCCACCUCUUCGACGACAAGGUCAUGAAGCAGCUCAAGCGCCGCCGCUACAC |
| | | CGGCUGGGGCCGCCUCAGCCGCAAGCUCAUCAAUGGGAUCCGAGACAAGCAGAGCGGCAAGACCAUCCUCGACUUCCUGA |
| | | AGAGCGACGGCUUCGCCAACCGCAACUUCAUGCAGCUCAUCCACGACGACAGCCUCACCUUCAAGGAGGACAUCCAGAAGG |
| | | CCCAGGUCAGCGGCCAGGGCGACAGCCUCCACGAGCACAUCGCCAAUCUCGCCGGGAGCCCAGCCAUCAAGAAGGGGAUCC |
| | | UCCAGACCGUCAAGGUCGUCGACGAGCUGGUCAAGGUCAUGGGCCGCCACAAGCCAGAGAACAUCGUCAUCGAGAUGGCC |
| | | AGGGAGAACCAGACCACUCAAAAGGGGCAGAAGAACAGCAGGGAGCGCAUGAAGCGCAUCGAGGAGGGCAUCAAGGAGCU |
| | | GGGCAGCCAGAUCCUCAAGGAGCACCCCGUCGAGAACACUCAACUCCAGAACGAGAAGCUCUACCUCUACUACCUCCAGAA |
| | | UGGGCGAGACAUGUACGUCGACCAGGAGCUGGACAUCAACCGCCUCAGCGACUACGACGUCGACCACAUCGUUCCCCAGA |
| | | GCUUCCUGAAGGACGACAGCAUCGACAACAAGGUCCUCACCCGAAGCGACAAGAACCGCGGCAAGAGCGACAACGUUCCC |
| | | UCAGAGGAAGUCGUCAAGAAGAUGAAGAACUACUGGCGCCAGCUCCUCAACGCCAAGCUCAUCACUCAACGCAAGUUCGA |
| | | CAAUCUCACCAAGGCGGAGCGCGGUGGCCUCAGCGAGCUGGACAAGGCCGGGUUCAUCAAGCGCCAGCUCGUCGAGACCC |
| | | GCCAGAUCACCAAGCACGUCGCCCAGAUCCUCGACAGCCGCAUGAACACCAAGUACGACGAGAACGACAAGCUCAUCAGGG |
| | | AAGUCAAGGUCAUCACCCUCAAGAGCAAGCUCGUCAGCGACUUCCGCAAGGACUUCCAGUUCUACAAGGUCAGGGAGAUC |
| | | AACAACUACCACCACGCCCACGACGCCUACCUCAACGCUGUGGUUGGCACCGCACUGAUCAAGAAGUACCCCAAGCUCGAG |
| | | AGCGAGUUCGUCUACGGCGACUACAAGGUCUACGACGUCCGCAAGAUGAUAGCCAAGAGCGAGCAGGAGAUCGGCAAGGC |
| | | CACCGCCAAGUACUUCUUCUACAGCAACAUCAUGAACUUCUUCAAGACAGAGAUCACCCUCGCCAAUGGUGAGAUCCGCA |
| | | AGCGCCCCCUCAUCGAGACCAAUGGUGAGACCGGUGAGAUCGUCUGGGACAAGGGGCGAGACUUCGCCACCGUCCGCAAG |
| | | GUCCUCAGCAUGCCCCAGGUGAACAUCGUCAAGAAGACAGAAGUCCAGACCGGUGGCUUCAGCAAGGAGAGCAUCCUUCC |
| | | AAAGCGCAACAGCGACAAGCUCAUCGCCCGCAAGAAGGACUGGGACCCCAAGAAGUACGGUGGCUUCGACAGCCCCACCG |
| | | UCGCCUACAGCGUCCUCGUCGUCGCCAAGGUCGAGAAGGGGAAGAGCAAGAAGCUCAAGAGCGUCAAGGAGCUGCUCGGC |
| | | AUCACCAUCAUGGAGCGAAGCAGCUUCGAGAAGAACCCCAUCGACUUCCUGGAAGCCAAGGGGUACAAGGAAGUCAAGAA |
| | | GGACCUCAUCAUCAAGCUUCCAAAGUACAGCCUCUUCGAGCUGGAGAAUGGGCGCAAGCGCAUGCUCGCCAGCGCCGGUG |
| | | AGCUGCAGAAGGGGAACGAGCUGGCACUUCCCUCAAAGUACGUCAACUUCCUGUACCUCGCCAGCCACUACGAGAAGCUC |
| | | AAGGGGAGCCCAGAGGACAACGAGCAGAAGCAGCUCUUCGUCGAGCAGCACAAGCACUACCUCGACGAGAUCAUCGAGCA |
| | | GAUCAGCGAGUUCAGCAAGCGCGUCAUCCUCGCCGACGCCAAUCUCGACAAGGUCCUCAGCGCCUACAACAAGCACCGAGA |
| | | CAAGCCCAUCAGGGAGCAGGCCGAGAACAUCAUCCACCUCUUCACCCUCACCAAUCUCGGUGCCCCAGCUGCCUUCAAGUA |
| | | CUUCGACACCACCAUCGACCGCAAGCGCUACACCUCGACUAAGGAAGUCCUCGACGCCACCCUCAUCCACCAGAGCAUCAC |
| | | CGGCCUCUACGAGACCCGCAUCGACCUCAGCCAGCUCGGUGGCGACGGUGGUGGCAGCCCCAAGAAGAAGCGCAAGGUCU |
| | | AGCUAGCACCAGCCUCAAGAACACCCGAAUGGAGUCUCUAAGCUACAUAAUACCAACUUACACUUUACAAAAUGUUGUCC |
| | | CCCAAAAUGUAGCCAUUCGUAUCUGCUCCUAAUAAAAAGAAAGUUUCUUCACAUUCUCUCGAGAAAAAAAAAAAAAAAAA |
| | | AAAAAAAAAAAAAAAAAAAAAAAAAAAAAAAAAAAAAAAAAAAAAAAAAAAAAAAAAAAAAAAAAAAAAAAAAAAAAA |
| | | UCUAG |
| 18 | Cas9 transcript | GGGAAGCUCAGAAUAAACGCUCAACUUUGGCCGGAUCUGCCACCAUGGACAAGAAGUACAGCAUCGGCCUGGACAUCGGC |
| | comprising SEQ | ACGAACAGCGUUGGCUGGGCUGUGAUCACGGACGAGUACAAGGUUCCCUCAAAGAAGUUCAAGGUGCUGGGCAACACGGA |
| | 8 | CCGGCACAGCAUCAAGAAGAAUCUCAUCGGUGCACUGCUGUUCGACAGCGGUGAGACGGCCGAAGCCACGCGGCUGAAGC |
| | | GGACGGCCCGCCGGCGGUACACGCGGCGGAAGAACCGGAUCUGCUACCUGCAGGAGAUCUUCAGCAACGAGAUGGCCAAG |
| | | GUGGACGACAGCUUCUUCCACCGGCUGGAGGAGAGCUUCCUGGUGGAGGAGGACAAGAAGCACGAGCGGCACCCCAUCUU |
| | | CGGCAACAUCGUGGACGAAGUCGCCUACCACGAGAAGUACCCCACCAUCUACCACCUGCGGAAGAAGCUGGUGGACUCGA |
| | | CUGACAAGGCCGACCUGCGGCUGAUCUACCUGGCACUGGCCCACAUGAUAAAGUUCCGGGGCCACUUCCUGAUCGAGGGC |
| | | GACCUGAACCCUGACAACAGCGACGUGGACAAGCUGUUCAUCCAGCUGGUGCAGACCUACAACCAGCUGUUCGAGGAGAA |
| | | CCCCAUCAACGCCAGCGGCGUGGACGCCAAGGCCAUCCUCAGCGCCCGCCUCAGCAAGAGCCGGCGGCUGGAGAAUCUCAU |
| | | CGCCCAGCUUCCAGGUGAGAAGAAGAAUGGGCUGUUCGGCAAUCUCAUCGCACUCAGCCUGGGCCUGACUCCCAACUUCA |
| | | AGAGCAACUUCGACCUGGCCGAGGACGCCAAGCUGCAGCUCAGCAAGGACACCUACGACGACGACCUGGACAAUCUCCUG |
| | | GCCCAGAUCGGCGACCAGUACGCCGACCUGUUCCUGGCUGCCAAGAAUCUCAGCGACGCCAUCCUGCUCAGCGACAUCCUG |
| | | CGGGUGAACACAGAGAUCACGAAGGCCCCCCUCAGCGCCAGCAUGAUAAAGCGGUACGACGAGCACCACCAGGACCUGAC |
| | | GCUGCUGAAGGCACUGGUGCGGCAGCAGCUUCCAGAGAAGUACAAGGAGAUCUUCUUCGACCAGAGCAAGAAUGGGUACG |
| | | CCGGGUACAUCGACGGUGGUGCCAGCCAGGAGGAGUUCUACAAGUUCAUCAAGCCCAUCCUGGAGAAGAUGGACGGCACA |
| | | GAGGAGCUGCUGGUGAAGCUGAACAGGGAGGACCUGCUGCGGAAGCAGCGGACGUUCGACAAUGGGAGCAUCCCCCACCA |
| | | GAUCCACCUGGGUGAGCUGCACGCCAUCCUGCGGCGGCAGGAGGACUUCUACCCCUUCCUGAAGGACAACAGGGAGAAGA |
| | | UCGAGAAGAUCCUGACGUUCCGGAUCCCCUACUACGUUGGCCCCCUGGCCCGCGGCAACAGCCGGUUCGCCUGGAUGACGC |
| | | GGAAGAGCGAGGAGACGAUCACUCCCUGGAACUUCGAGGAAGUCGUGGACAAGGGUGCCAGCGCCCAGAGCUUCAUCGAG |
| | | CGGAUGACGAACUUCGACAAGAAUCUUCCAAACGAGAAGGUGCUUCCAAAGCACAGCCUGCUGUACGAGUACUUCACGGU |
| | | GUACAACGAGCUGACGAAGGUGAAGUACGUGACAGAGGGCAUGCGGAAGCCCGCCUUCCUCAGCGGUGAGCAGAAGAAGG |
| | | CCAUCGUGGACCUGCUGUUCAAGACGAACCGGAAGGUGACGGUGAAGCAGCUGAAGGAGGACUACUUCAAGAAGAUCGAG |
| | | UGCUUCGACAGCGUGGAGAUCAGCGGCGUGGAGGACCGGUUCAACGCCAGCCUGGGCACCUACCACGACCUGCUGAAGAU |
| | | CAUCAAGGACAAGGACUUCCUGGACAACGAGGAGAACGAGGACAUCCUGGAGGACAUCGUGCUGACGCUGACGCUGUUCG |
| | | AGGACAGGGAGAUGAUAGAGGAGCGGCUGAAGACCUACGCCCACCUGUUCGACGACAAGGUGAUGAAGCAGCUGAAGCGG |
| | | CGGCGGUACACGGGCUGGGGCCGGCUCAGCCGGAAGCUGAUCAAUGGGAUCCGAGACAAGCAGAGCGGCAAGACGAUCCU |
| | | GGACUUCCUGAAGAGCGACGGCUUCGCCAACCGGAACUUCAUGCAGCUGAUCCACGACGACAGCCUGACGUUCAAGGAGG |
| | | ACAUCCAGAAGGCCCAGGUCAGCGGCCAGGGCGACAGCCUGCACGAGCACAUCGCCAAUCUCGCCGGGAGCCCCGCCAUCA |
| | | AGAAGGGGAUCCUGCAGACGGUGAAGGUGGUGGACGAGCUGGUGAAGGUGAUGGGCCGGCACAAGCCAGAGAACAUCGU |
| | | GAUCGAGAUGGCCAGGGAGAACCAGACGACUCAAAAGGGGCAGAAGAACAGCAGGGAGCGGAUGAAGCGGAUCGAGGAG |
| | | GGCAUCAAGGAGCUGGGCAGCCAGAUCCUGAAGGAGCACCCCGUGGAGAACACUCAACUGCAGAACGAGAAGCUGUACCU |
| | | GUACUACCUGCAGAAUGGGCGAGACAUGUACGUGGACCAGGAGCUGGACAUCAACCGGCUCAGCGACUACGACGUGGACC |
| | | ACAUCGUUCCCCAGAGCUUCCUGAAGGACGACAGCAUCGACAACAAGGUGCUGACGCGGAGCGACAAGAACCGGGGCAAG |
| | | AGCGACAACGUUCCCUCAGAGGAAGUCGUGAAGAAGAUGAAGAACUACUGGCGGCAGCUGCUGAACGCCAAGCUGAUCAC |
| | | UCAACGGAAGUUCGACAAUCUCACGAAGGCCGAGCGGGGUGGCCUCAGCGAGCUGGACAAGGCCGGGUUCAUCAAGCGGC |
| | | AGCUGGUGGAGACGCGGCAGAUCACGAAGCACGUGGCCCAGAUCCUGGACAGCCGGAUGAACACGAAGUACGACGAGAAC |
| | | GACAAGCUGAUCAGGGAAGUCAAGGUGAUCACGCUGAAGAGCAAGCUGGUCAGCGACUUCCGGAAGGACUUCCAGUUCUA |
| | | CAAGGUGAGGGAGAUCAACAACUACCACCACGCCCACGACGCCUACCUGAACGCUGUGGUUGGCACGGCACUGAUCAAGA |
| | | AGUACCCCAAGCUGGAGAGCGAGUUCGUGUACGGCGACUACAAGGUGUACGACGUGCGGAAGAUGAUAGCCAAGAGCGAG |
| | | CAGGAGAUCGGCAAGGCCACGGCCAAGUACUUCUUCUACAGCAACAUCAUGAACUUCUUCAAGACAGAGAUCACGCUGGC |
| | | CAAUGGUGAGAUCCGGAAGCGGCCCCUGAUCGAGACGAAUGGUGAGACGGGUGAGAUCGUGUGGGACAAGGGGCGAGACU |
| | | UCGCCACGGUGCGGAAGGUGCUCAGCAUGCCCCAGGUGAACAUCGUGAAGAAGACAGAAGUCCAGACGGGUGGCUUCAGC |
| | | AAGGAGAGCAUCCUUCCAAAGCGGAACAGCGACAAGCUGAUCGCCCGCAAGAAGGACUGGGACCCCAAGAAGUACGGUGG |
| | | CUUCGACAGCCCCACCGUGGCCUACAGCGUGCUGGUGGUGGCCAAGGUGGAGAAGGGGAAGAGCAAGAAGCUGAAGAGCG |
| | | UGAAGGAGCUGCUGGGCAUCACGAUCAUGGAGCGGAGCAGCUUCGAGAAGAACCCCAUCGACUUCCUGGAAGCCAAGGGG |
| | | UACAAGGAAGUCAAGAAGGACCUGAUCAUCAAGCUUCCAAAGUACAGCCUGUUCGAGCUGGAGAAUGGGCGGAAGCGGAU |
| | | GCUGGCCAGCGCCGGUGAGCUGCAGAAGGGGAACGAGCUGGCACUUCCCUCAAAGUACGUGAACUUCCUGUACCUGGCCA |
| | | GCCACUACGAGAAGCUGAAGGGGAGCCCAGAGGACAACGAGCAGAAGCAGCUGUUCGUGGAGCAGCACAAGCACUACCUG |
| | | GACGAGAUCAUCGAGCAGAUCAGCGAGUUCAGCAAGCGGGUGAUCCUGGCCGACGCCAAUCUCGACAAGGUGCUCAGCGC |
| | | CUACAACAAGCACCGAGACAAGCCCAUCAGGGAGCAGGCCGAGAACAUCAUCCACCUGUUCACGCUGACGAAUCUCGGUG |
| | | CCCCCGCUGCCUUCAAGUACUUCGACACGACGAUCGACCGGAAGCGGUACACGUCGACUAAGGAAGUCCUGGACGCCACGC |
| | | UGAUCCACCAGAGCAUCACGGGCCUGUACGAGACGCGGAUCGACCUCAGCCAGCUGGGUGGCGACGGUGGUGGCAGCCCC |
| | | AAGAAGAAGCGGAAGGUGUAGCUAGCACCAGCCUCAAGAACACCCGAAUGGAGUCUCUAAGCUACAUAAUACCAACUUAC |
| | | ACUUUACAAAAUGUUGUCCCCCAAAAUGUAGCCAUUCGUAUCUGCUCCUAAUAAAAAGAAAGUUUCUUCACAUUCUCUCG |
| | | AGAAAAAAAAAAAAAAAAAAAAAAAAAAAAAAAAAAAAAAAAAAAAAAAAAAAAAAAAAAAAAAAAAAAAAAAAAAAA |
| | | AAAAAAAAAAAAAAAAAAAUCUAG |
| 19 | Cas9 mRNA | GGGAAGCUCAGAAUAAACGCUCAACUUUGGCCGGAUCUGCCACCAUGGACAAGAAGUACAGCAUCGGCCUCGACAUCGGC |
| | transcript | ACCAACAGCGUCGGCUGGGCCGUCAUCACCGACGAGUACAAGGUCCCCAGCAAGAAGUUCAAGGUCCUCGGCAACACCGAC |
| | comprising SEQ | CGCCACAGCAUCAAGAAGAACCUCAUCGGCGCCCUCCUCUUCGACAGCGGCGAGACCGCCGAGGCCACCCGCCUCAAGCGC |
| | 10 | ACCGCCCGCCGCCGCUACACCCGCCGCAAGAACCGCAUCUGCUACCUCCAGGAGAUCUUCAGCAACGAGAUGGCCAAGGUC |
| | | GACGACAGCUUCUUCCACCGCCUCGAGGAGAGCUUCCUCGUCGAGGAGGACAAGAAGCACGAGCGCCACCCCAUCUUCGGC |
| | | AACAUCGUCGACGAGGUCGCCUACCACGAGAAGUACCCCACCAUCUACCACCUCCGCAAGAAGCUCGUCGACAGCACCGAC |
| | | AAGGCCGACCUCCGCCUCAUCUACCUCGCCCUCGCCCACAUGAUCAAGUUCCGCGGCCACUUCCUCAUCGAGGGCGACCUC |
| | | AACCCCGACAACAGCGACGUCGACAAGCUCUUCAUCCAGCUCGUCCAGACCUACAACCAGCUCUUCGAGGAGAACCCCAUC |
| | | AACGCCAGCGGCGUCGACGCCAAGGCCAUCCUCAGCGCCCGCCUCAGCAAGAGCCGCCGCCUCGAGAACCUCAUCGCCCAG |
| | | CUCCCCGGCGAGAAGAAGAACGGCCUCUUCGGCAACCUCAUCGCCCUCAGCCUCGGCCUCACCCCCAACUUCAAGAGCAAC |
| | | UUCGACCUCGCCGAGGACGCCAAGCUCCAGCUCAGCAAGGACACCUACGACGACGACCUCGACAACCUCCUCGCCCAGAUC |
| | | GGCGACCAGUACGCCGACCUCUUCCUCGCCGCCAAGAACCUCAGCGACGCCAUCCUCCUCAGCGACAUCCUCCGCGUCAAC |
| | | ACCGAGAUCACCAAGGCCCCCCUCAGCGCCAGCAUGAUCAAGCGCUACGACGAGCACCACCAGGACCUCACCCUCCUCAAG |
| | | GCCCUCGUCCGCCAGCAGCUCCCCGAGAAGUACAAGGAGAUCUUCUUCGACCAGAGCAAGAACGGCUACGCCGGCUACAUC |
| | | GACGGCGGCGCCAGCCAGGAGGAGUUCUACAAGUUCAUCAAGCCCAUCCUCGAGAAGAUGGACGGCACCGAGGAGCUCCU |
| | | CGUCAAGCUCAACCGCGAGGACCUCCUCCGCAAGCAGCGCACCUUCGACAACGGCAGCAUCCCCCACCAGAUCCACCUCGG |
| | | CGAGCUCCACGCCAUCCUCCGCCGCCAGGAGGACUUCUACCCCUUCCUCAAGGACAACCGCGAGAAGAUCGAGAAGAUCCU |
| | | CACCUUCCGCAUCCCCUACUACGUCGGCCCCCUCGCCCGCGGCAACAGCCGCUUCGCCUGGAUGACCCGCAAGAGCGAGGA |
| | | GACCAUCACCCCCUGGAACUUCGAGGAGGUCGUCGACAAGGGCGCCAGCGCCCAGAGCUUCAUCGAGCGCAUGACCAACUU |
| | | CGACAAGAACCUCCCCAACGAGAAGGUCCUCCCCAAGCACAGCCUCCUCUACGAGUACUUCACCGUCUACAACGAGCUCAC |
| | | CAAGGUCAAGUACGUCACCGAGGGCAUGCGCAAGCCCGCCUUCCUCAGCGGCGAGCAGAAGAAGGCCAUCGUCGACCUCCU |
| | | CUUCAAGACCAACCGCAAGGUCACCGUCAAGCAGCUCAAGGAGGACUACUUCAAGAAGAUCGAGUGCUUCGACAGCGUCG |
| | | AGAUCAGCGGCGUCGAGGACCGCUUCAACGCCAGCCUCGGCACCUACCACGACCUCCUCAAGAUCAUCAAGGACAAGGACU |
| | | UCCUCGACAACGAGGAGAACGAGGACAUCCUCGAGGACAUCGUCCUCACCCUCACCCUCUUCGAGGACCGCGAGAUGAUCG |
| | | AGGAGCGCCUCAAGACCUACGCCCACCUCUUCGACGACAAGGUCAUGAAGCAGCUCAAGCGCCGCCGCUACACCGGCUGGG |
| | | GCCGCCUCAGCCGCAAGCUCAUCAACGGCAUCCGCGACAAGCAGAGCGGCAAGACCAUCCUCGACUUCCUCAAGAGCGACG |
| | | GCUUCGCCAACCGCAACUUCAUGCAGCUCAUCCACGACGACAGCCUCACCUUCAAGGAGGACAUCCAGAAGGCCCAGGUCA |
| | | GCGGCCAGGGCGACAGCCUCCACGAGCACAUCGCCAACCUCGCCGGCAGCCCCGCCAUCAAGAAGGGCAUCCUCCAGACCG |
| | | UCAAGGUCGUCGACGAGCUCGUCAAGGUCAUGGGCCGCCACAAGCCCGAGAACAUCGUCAUCGAGAUGGCCCGCGAGAAC |
| | | CAGACCACCCAGAAGGGCCAGAAGAACAGCCGCGAGCGCAUGAAGCGCAUCGAGGAGGGCAUCAAGGAGCUCGGCAGCCA |
| | | GAUCCUCAAGGAGCACCCCGUCGAGAACACCCAGCUCCAGAACGAGAAGCUCUACCUCUACUACCUCCAGAACGGCCGCGA |
| | | CAUGUACGUCGACCAGGAGCUCGACAUCAACCGCCUCAGCGACUACGACGUCGACCACAUCGUCCCCCAGAGCUUCCUCAA |
| | | GGACGACAGCAUCGACAACAAGGUCCUCACCCGCAGCGACAAGAACCGCGGCAAGAGCGACAACGUCCCCAGCGAGGAGG |
| | | UCGUCAAGAAGAUGAAGAACUACUGGCGCCAGCUCCUCAACGCCAAGCUCAUCACCCAGCGCAAGUUCGACAACCUCACCA |
| | | AGGCCGAGCGCGGCGGCCUCAGCGAGCUCGACAAGGCCGGCUUCAUCAAGCGCCAGCUCGUCGAGACCCGCCAGAUCACCA |
| | | AGCACGUCGCCCAGAUCCUCGACAGCCGCAUGAACACCAAGUACGACGAGAACGACAAGCUCAUCCGCGAGGUCAAGGUC |
| | | AUCACCCUCAAGAGCAAGCUCGUCAGCGACUUCCGCAAGGACUUCCAGUUCUACAAGGUCCGCGAGAUCAACAACUACCAC |
| | | CACGCCCACGACGCCUACCUCAACGCCGUCGUCGGCACCGCCCUCAUCAAGAAGUACCCCAAGCUCGAGAGCGAGUUCGUC |
| | | UACGGCGACUACAAGGUCUACGACGUCCGCAAGAUGAUCGCCAAGAGCGAGCAGGAGAUCGGCAAGGCCACCGCCAAGUA |
| | | CUUCUUCUACAGCAACAUCAUGAACUUCUUCAAGACCGAGAUCACCCUCGCCAACGGCGAGAUCCGCAAGCGCCCCCUCAU |
| | | CGAGACCAACGGCGAGACCGGCGAGAUCGUCUGGGACAAGGGCCGCGACUUCGCCACCGUCCGCAAGGUCCUCAGCAUGCC |
| | | CCAGGUCAACAUCGUCAAGAAGACCGAGGUCCAGACCGGCGGCUUCAGCAAGGAGAGCAUCCUCCCCAAGCGCAACAGCG |
| | | ACAAGCUCAUCGCCCGCAAGAAGGACUGGGACCCCAAGAAGUACGGCGGCUUCGACAGCCCCACCGUCGCCUACAGCGUCC |
| | | UCGUCGUCGCCAAGGUCGAGAAGGGCAAGAGCAAGAAGCUCAAGAGCGUCAAGGAGCUCCUCGGCAUCACCAUCAUGGAG |
| | | CGCAGCAGCUUCGAGAAGAACCCCAUCGACUUCCUCGAGGCCAAGGGCUACAAGGAGGUCAAGAAGGACCUCAUCAUCAA |
| | | GCUCCCCAAGUACAGCCUCUUCGAGCUCGAGAACGGCCGCAAGCGCAUGCUCGCCAGCGCCGGCGAGCUCCAGAAGGGCAA |
| | | CGAGCUCGCCCUCCCCAGCAAGUACGUCAACUUCCUCUACCUCGCCAGCCACUACGAGAAGCUCAAGGGCAGCCCCGAGGA |
| | | CAACGAGCAGAAGCAGCUCUUCGUCGAGCAGCACAAGCACUACCUCGACGAGAUCAUCGAGCAGAUCAGCGAGUUCAGCA |
| | | AGCGCGUCAUCCUCGCCGACGCCAACCUCGACAAGGUCCUCAGCGCCUACAACAAGCACCGCGACAAGCCCAUCCGCGAGC |
| | | AGGCCGAGAACAUCAUCCACCUCUUCACCCUCACCAACCUCGGCGCCCCCGCCGCCUUCAAGUACUUCGACACCACCAUCG |
| | | ACCGCAAGCGCUACACCAGCACCAAGGAGGUCCUCGACGCCACCCUCAUCCACCAGAGCAUCACCGGCCUCUACGAGACCC |
| | | GCAUCGACCUCAGCCAGCUCGGCGGCGACGGCGGCGGCAGCCCCAAGAAGAAGCGCAAGGUCUAGCUAGCACCAGCCUCAA |
| | | GAACACCCGAAUGGAGUCUCUAAGCUACAUAAUACCAACUUACACUUUACAAAAUGUUGUCCCCCAAAAUGUAGCCAUUC |
| | | GUAUCUGCUCCUAAUAAAAAGAAAGUUUCUUCACAUUCUCUCGAGAAAAAAAAAAAAAAAAAAAAAAAAAAAAAAAAAA |
| | | AAAAAAAAAAAAAAAAAAAAAAAAAAAAAAAAAAAAAAAAAAAAAAAAAAAAAAAAAAAAAAAAAAAAAAUCUAG |
| 20 | Cas9 transcript | GGGAAGCUCAGAAUAAACGCUCAACUUUGGCCGGAUCUGCCACCAUGGACAAGAAGUACAGCAUCGGCCUGGACAUCGGC |
| | comprising SEQ | ACGAACAGCGUUGGCUGGGCUGUGAUCACGGACGAGUACAAGGUUCCCAGCAAGAAGUUCAAGGUGCUGGGCAACACGGA |
| | 6 | CCGGCACAGCAUCAAGAAGAAUCUGAUCGGUGCACUGCUGUUCGACAGCGGUGAGACGGCCGAAGCCACGCGGCUGAAGC |
| | | GGACGGCCCGGCGGCGGUACACGCGGCGGAAGAACCGGAUCUGCUACCUGCAGGAGAUCUUCAGCAACGAGAUGGCCAAG |
| | | GUGGACGACAGCUUCUUCCACCGGCUGGAGGAGAGCUUCCUGGUGGAGGAGGACAAGAAGCACGAGCGGCACCCCAUCUU |
| | | CGGCAACAUCGUGGACGAAGUGGCCUACCACGAGAAGUACCCCACGAUCUACCACCUGCGGAAGAAGCUGGUGGACUCGA |
| | | CGGACAAGGCCGACCUGCGGCUGAUCUACCUGGCACUGGCCCACAUGAUCAAGUUCCGGGGCCACUUCCUGAUCGAGGGC |
| | | GACCUGAACCCUGACAACAGCGACGUGGACAAGCUGUUCAUCCAGCUGGUGCAGACCUACAACCAGCUGUUCGAGGAGAA |
| | | CCCCAUCAACGCCAGCGGCGUGGACGCCAAGGCCAUCCUCAGCGCCCGGCUCAGCAAGAGCCGGCGGCUGGAGAAUCUGAU |
| | | CGCCCAGCUUCCCGGUGAGAAGAAGAAUGGCCUGUUCGGCAAUCUGAUCGCACUCAGCCUGGGCCUGACUCCCAACUUCA |
| | | AGAGCAACUUCGACCUGGCCGAGGACGCCAAGCUGCAGCUCAGCAAGGACACCUACGACGACGACCUGGACAAUCUGCUG |
| | | GCCCAGAUCGGCGACCAGUACGCCGACCUGUUCCUGGCUGCCAAGAAUCUCAGCGACGCCAUCCUGCUCAGCGACAUCCUG |
| | | CGGGUGAACACAGAGAUCACGAAGGCCCCCCUCAGCGCCAGCAUGAUCAAGCGGUACGACGAGCACCACCAGGACCUGACG |
| | | CUGCUGAAGGCACUGGUGCGGCAGCAGCUUCCAGAGAAGUACAAGGAGAUCUUCUUCGACCAGAGCAAGAAUGGCUACGC |
| | | CGGCUACAUCGACGGUGGUGCCAGCCAGGAGGAGUUCUACAAGUUCAUCAAGCCCAUCCUGGAGAAGAUGGACGGCACAG |
| | | AGGAGCUGCUGGUGAAGCUGAACAGGGAGGACCUGCUGCGGAAGCAGCGGACGUUCGACAAUGGCAGCAUCCCCCACCAG |
| | | AUCCACCUGGGUGAGCUGCACGCCAUCCUGCGGCGGCAGGAGGACUUCUACCCCUUCCUGAAGGACAACAGGGAGAAGAU |
| | | CGAGAAGAUCCUGACGUUCCGGAUCCCCUACUACGUUGGCCCCCUGGCCCGGGGCAACAGCCGGUUCGCCUGGAUGACGCG |
| | | GAAGAGCGAGGAGACGAUCACUCCCUGGAACUUCGAGGAAGUGGUGGACAAGGGUGCCAGCGCCCAGAGCUUCAUCGAGC |
| | | GGAUGACGAACUUCGACAAGAAUCUUCCCAACGAGAAGGUGCUUCCCAAGCACAGCCUGCUGUACGAGUACUUCACGGUG |
| | | UACAACGAGCUGACGAAGGUGAAGUACGUGACAGAGGGCAUGCGGAAGCCCGCCUUCCUCAGCGGUGAGCAGAAGAAGGC |
| | | CAUCGUGGACCUGCUGUUCAAGACGAACCGGAAGGUGACGGUGAAGCAGCUGAAGGAGGACUACUUCAAGAAGAUCGAGU |
| | | GCUUCGACAGCGUGGAGAUCAGCGGCGUGGAGGACCGGUUCAACGCCAGCCUGGGCACCUACCACGACCUGCUGAAGAUC |
| | | AUCAAGGACAAGGACUUCCUGGACAACGAGGAGAACGAGGACAUCCUGGAGGACAUCGUGCUGACGCUGACGCUGUUCGA |
| | | GGACAGGGAGAUGAUCGAGGAGCGGCUGAAGACCUACGCCCACCUGUUCGACGACAAGGUGAUGAAGCAGCUGAAGCGGC |
| | | GGCGGUACACGGGCUGGGGCCGGCUCAGCCGGAAGCUGAUCAAUGGCAUCCGAGACAAGCAGAGCGGCAAGACGAUCCUG |
| | | GACUUCCUGAAGAGCGACGGCUUCGCCAACCGGAACUUCAUGCAGCUGAUCCACGACGACAGCCUGACGUUCAAGGAGGA |
| | | CAUCCAGAAGGCCCAGGUCAGCGGCCAGGGCGACAGCCUGCACGAGCACAUCGCCAAUCUGGCCGGCAGCCCCGCCAUCAA |
| | | GAAGGGCAUCCUGCAGACGGUGAAGGUGGUGGACGAGCUGGUGAAGGUGAUGGGCCGGCACAAGCCAGAGAACAUCGUGA |
| | | UCGAGAUGGCCAGGGAGAACCAGACGACUCAGAAGGGCCAGAAGAACAGCAGGGAGCGGAUGAAGCGGAUCGAGGAGGGC |
| | | AUCAAGGAGCUGGGCAGCCAGAUCCUGAAGGAGCACCCCGUGGAGAACACUCAGCUGCAGAACGAGAAGCUGUACCUGUA |
| | | CUACCUGCAGAAUGGCCGAGACAUGUACGUGGACCAGGAGCUGGACAUCAACCGGCUCAGCGACUACGACGUGGACCACA |
| | | UCGUUCCCCAGAGCUUCCUGAAGGACGACAGCAUCGACAACAAGGUGCUGACGCGGAGCGACAAGAACCGGGGCAAGAGC |
| | | GACAACGUUCCCAGCGAGGAAGUGGUGAAGAAGAUGAAGAACUACUGGCGGCAGCUGCUGAACGCCAAGCUGAUCACUCA |
| | | GCGGAAGUUCGACAAUCUGACGAAGGCCGAGCGGGGUGGCCUCAGCGAGCUGGACAAGGCCGGCUUCAUCAAGCGGCAGC |
| | | UGGUGGAGACGCGGCAGAUCACGAAGCACGUGGCCCAGAUCCUGGACAGCCGGAUGAACACGAAGUACGACGAGAACGAC |
| | | AAGCUGAUCAGGGAAGUGAAGGUGAUCACGCUGAAGAGCAAGCUGGUCAGCGACUUCCGGAAGGACUUCCAGUUCUACAA |
| | | GGUGAGGGAGAUCAACAACUACCACCACGCCCACGACGCCUACCUGAACGCUGUGGUUGGCACGGCACUGAUCAAGAAGU |
| | | ACCCCAAGCUGGAGAGCGAGUUCGUGUACGGCGACUACAAGGUGUACGACGUGCGGAAGAUGAUCGCCAAGAGCGAGCAG |
| | | GAGAUCGGCAAGGCCACGGCCAAGUACUUCUUCUACAGCAACAUCAUGAACUUCUUCAAGACAGAGAUCACGCUGGCCAA |
| | | UGGUGAGAUCCGGAAGCGGCCCCUGAUCGAGACGAAUGGUGAGACGGGUGAGAUCGUGUGGGACAAGGGCCGAGACUUCG |
| | | CCACGGUGCGGAAGGUGCUCAGCAUGCCCCAGGUGAACAUCGUGAAGAAGACAGAAGUGCAGACGGGUGGCUUCAGCAAG |
| | | GAGAGCAUCCUUCCCAAGCGGAACAGCGACAAGCUGAUCGCCCGGAAGAAGGACUGGGACCCCAAGAAGUACGGUGGCUU |
| | | CGACAGCCCCACGGUGGCCUACAGCGUGCUGGUGGUGGCCAAGGUGGAGAAGGGCAAGAGCAAGAAGCUGAAGAGCGUGA |
| | | AGGAGCUGCUGGGCAUCACGAUCAUGGAGCGGAGCAGCUUCGAGAAGAACCCCAUCGACUUCCUGGAAGCCAAGGGCUAC |
| | | AAGGAAGUGAAGAAGGACCUGAUCAUCAAGCUUCCCAAGUACAGCCUGUUCGAGCUGGAGAAUGGCCGGAAGCGGAUGCU |
| | | GGCCAGCGCCGGUGAGCUGCAGAAGGGCAACGAGCUGGCACUUCCCAGCAAGUACGUGAACUUCCUGUACCUGGCCAGCC |
| | | ACUACGAGAAGCUGAAGGGCAGCCCAGAGGACAACGAGCAGAAGCAGCUGUUCGUGGAGCAGCACAAGCACUACCUGGAC |
| | | GAGAUCAUCGAGCAGAUCAGCGAGUUCAGCAAGCGGGUGAUCCUGGCCGACGCCAAUCUGGACAAGGUGCUCAGCGCCUA |
| | | CAACAAGCACCGAGACAAGCCCAUCAGGGAGCAGGCCGAGAACAUCAUCCACCUGUUCACGCUGACGAAUCUGGGUGCCCC |
| | | CGCUGCCUUCAAGUACUUCGACACGACGAUCGACCGGAAGCGGUACACGUCGACGAAGGAAGUGCUGGACGCCACGCUGA |
| | | UCCACCAGAGCAUCACGGGCCUGUACGAGACGCGGAUCGACCUCAGCCAGCUGGGUGGCGACGGUGGUGGCAGCCCCAAG |
| | | AAGAAGCGGAAGGUGUAGCUAGCACCAGCCUCAAGAACACCCGAAUGGAGUCUCUAAGCUACAUAAUACCAACUUACACU |
| | | UUACAAAAUGUUGUCCCCCAAAAUGUAGCCAUUCGUAUCUGCUCCUAAUAAAAAGAAAGUUUCUUCACAUUCUCUCGAGA |
| | | AAAAAAAAAAAAAAAAAAAAAAAAAAAAAAAAAAAAAAAAAAAAAAAAAAAAAAAAAAAAAAAAAAAAAAAAAAAAAA |
| | | AAAAAAAAAAAAAAAAAAAAAAUCUAG |
| 21 | Cas9 mRNA | GGGAAGCUCAGAAUAAACGCUCAACUUUGGCCGGAUCUGCCACCAUGGACAAAAAAUACUCAAUAGGCCUCGACAUAGGC |
| | transcript | ACCAACUCAGUCGGCUGGGCCGUCAUAACCGACGAGUACAAAGUCCCCUCAAAAAAAUUCAAAGUCCUCGGCAACACCGA |
| | comprising SEQ | CAGGCACUCAAUAAAAAAAAACCUCAUAGGCGCCCUCCUCUUCGACUCAGGCGAGACCGCCGAGGCCACCAGGCUCAAAAG |
| | 11 | GACCGCCAGGAGGAGGUACACCAGGAGGAAAAACAGGAUAUGCUACCUCCAGGAGAUAUUCUCAAACGAGAUGGCCAAAG |
| | | UCGACGACUCAUUCUUCCACAGGCUCGAGGAGUCAUUCCUCGUCGAGGAGGACAAAAAACACGAGAGGCACCCCAUAUUC |
| | | GGCAACAUAGUCGACGAGGUCGCCUACCACGAGAAAUACCCCACCAUAUACCACCUCAGGAAAAAACUCGUCGACUCAACC |
| | | GACAAAGCCGACCUCAGGCUCAUAUACCUCGCCCUCGCCCACAUGAUAAAAUUCAGGGGCCACUUCCUCAUAGAGGGCGAC |
| | | CUCAACCCCGACAACUCAGACGUCGACAAACUCUUCAUACAGCUCGUCCAGACCUACAACCAGCUCUUCGAGGAGAACCCC |
| | | AUAAACGCCUCAGGCGUCGACGCCAAAGCCAUACUCUCAGCCAGGCUCUCAAAAUCAAGGAGGCUCGAGAACCUCAUAGC |
| | | CCAGCUCCCCGGCGAGAAAAAAAACGGCCUCUUCGGCAACCUCAUAGCCCUCUCACUCGGCCUCACCCCCAACUUCAAAUC |
| | | AAACUUCGACCUCGCCGAGGACGCCAAACUCCAGCUCUCAAAAGACACCUACGACGACGACCUCGACAACCUCCUCGCCCA |
| | | GAUAGGCGACCAGUACGCCGACCUCUUCCUCGCCGCCAAAAACCUCUCAGACGCCAUACUCCUCUCAGACAUACUCAGGGU |
| | | CAACACCGAGAUAACCAAAGCCCCCCUCUCAGCCUCAAUGAUAAAAAGGUACGACGAGCACCACCAGGACCUCACCCUCCU |
| | | CAAAGCCCUCGUCAGGCAGCAGCUCCCCGAGAAAUACAAAGAGAUAUUCUUCGACCAGUCAAAAAACGGCUACGCCGGCU |
| | | ACAUAGACGGCGGCGCCUCACAGGAGGAGUUCUACAAAUUCAUAAAACCCAUACUCGAGAAAAUGGACGGCACCGAGGAG |
| | | CUCCUCGUCAAACUCAACAGGGAGGACCUCCUCAGGAAACAGAGGACCUUCGACAACGGCUCAAUACCCCACCAGAUACAC |
| | | CUCGGCGAGCUCCACGCCAUACUCAGGAGGCAGGAGGACUUCUACCCCUUCCUCAAAGACAACAGGGAGAAAAUAGAGAA |
| | | AAUACUCACCUUCAGGAUACCCUACUACGUCGGCCCCCUCGCCAGGGGCAACUCAAGGUUCGCCUGGAUGACCAGGAAAUC |
| | | AGAGGAGACCAUAACCCCCUGGAACUUCGAGGAGGUCGUCGACAAAGGCGCCUCAGCCCAGUCAUUCAUAGAGAGGAUGA |
| | | CCAACUUCGACAAAAACCUCCCCAACGAGAAAGUCCUCCCCAAACACUCACUCCUCUACGAGUACUUCACCGUCUACAACG |
| | | AGCUCACCAAAGUCAAAUACGUCACCGAGGGCAUGAGGAAACCCGCCUUCCUCUCAGGCGAGCAGAAAAAAGCCAUAGUC |
| | | GACCUCCUCUUCAAAACCAACAGGAAAGUCACCGUCAAACAGCUCAAAGAGGACUACUUCAAAAAAAUAGAGUGCUUCGA |
| | | CUCAGUCGAGAUAUCAGGCGUCGAGGACAGGUUCAACGCCUCACUCGGCACCUACCACGACCUCCUCAAAAUAAUAAAAG |
| | | ACAAAGACUUCCUCGACAACGAGGAGAACGAGGACAUACUCGAGGACAUAGUCCUCACCCUCACCCUCUUCGAGGACAGG |
| | | GAGAUGAUAGAGGAGAGGCUCAAAACCUACGCCCACCUCUUCGACGACAAAGUCAUGAAACAGCUCAAAAGGAGGAGGUA |
| | | CACCGGCUGGGGCAGGCUCUCAAGGAAACUCAUAAACGGCAUAAGGGACAAACAGUCAGGCAAAACCAUACUCGACUUCC |
| | | UCAAAUCAGACGGCUUCGCCAACAGGAACUUCAUGCAGCUCAUACACGACGACUCACUCACCUUCAAAGAGGACAUACAG |
| | | AAAGCCCAGGUCUCAGGCCAGGGCGACUCACUCCACGAGCACAUAGCCAACCUCGCCGGCUCACCCGCCAUAAAAAAAGGC |
| | | AUACUCCAGACCGUCAAAGUCGUCGACGAGCUCGUCAAAGUCAUGGGCAGGCACAAACCCGAGAACAUAGUCAUAGAGAU |
| | | GGCCAGGGAGAACCAGACCACCCAGAAAGGCCAGAAAAACUCAAGGGAGAGGAUGAAAAGGAUAGAGGAGGGCAUAAAA |
| | | GAGCUCGGCUCACAGAUACUCAAAGAGCACCCCGUCGAGAACACCCAGCUCCAGAACGAGAAACUCUACCUCUACUACCUC |
| | | CAGAACGGCAGGGACAUGUACGUCGACCAGGAGCUCGACAUAAACAGGCUCUCAGACUACGACGUCGACCACAUAGUCCC |
| | | CCAGUCAUUCCUCAAAGACGACUCAAUAGACAACAAAGUCCUCACCAGGUCAGACAAAAACAGGGGCAAAUCAGACAACG |
| | | UCCCCUCAGAGGAGGUCGUCAAAAAAAUGAAAAACUACUGGAGGCAGCUCCUCAACGCCAAACUCAUAACCCAGAGGAAA |
| | | UUCGACAACCUCACCAAAGCCGAGAGGGGCGGCCUCUCAGAGCUCGACAAAGCCGGCUUCAUAAAAAGGCAGCUCGUCGA |
| | | GACCAGGCAGAUAACCAAACACGUCGCCCAGAUACUCGACUCAAGGAUGAACACCAAAUACGACGAGAACGACAAACUCA |
| | | UAAGGGAGGUCAAAGUCAUAACCCUCAAAUCAAAACUCGUCUCAGACUUCAGGAAAGACUUCCAGUUCUACAAAGUCAGG |
| | | GAGAUAAACAACUACCACCACGCCCACGACGCCUACCUCAACGCCGUCGUCGGCACCGCCCUCAUAAAAAAAUACCCCAAA |
| | | CUCGAGUCAGAGUUCGUCUACGGCGACUACAAAGUCUACGACGUCAGGAAAAUGAUAGCCAAAUCAGAGCAGGAGAUAGG |
| | | CAAAGCCACCGCCAAAUACUUCUUCUACUCAAACAUAAUGAACUUCUUCAAAACCGAGAUAACCCUCGCCAACGGCGAGA |
| | | UAAGGAAAAGGCCCCUCAUAGAGACCAACGGCGAGACCGGCGAGAUAGUCUGGGACAAAGGCAGGGACUUCGCCACCGUC |
| | | AGGAAAGUCCUCUCAAUGCCCCAGGUCAACAUAGUCAAAAAAACCGAGGUCCAGACCGGCGGCUUCUCAAAAGAGUCAAU |
| | | ACUCCCCAAAAGGAACUCAGACAAACUCAUAGCCAGGAAAAAAGACUGGGACCCCAAAAAAUACGGCGGCUUCGACUCAC |
| | | CCACCGUCGCCUACUCAGUCCUCGUCGUCGCCAAAGUCGAGAAAGGCAAAUCAAAAAAACUCAAAUCAGUCAAAGAGCUC |
| | | CUCGGCAUAACCAUAAUGGAGAGGUCAUCAUUCGAGAAAAACCCCAUAGACUUCCUCGAGGCCAAAGGCUACAAAGAGGU |
| | | CAAAAAAGACCUCAUAAUAAAACUCCCCAAAUACUCACUCUUCGAGCUCGAGAACGGCAGGAAAAGGAUGCUCGCCUCAG |
| | | CCGGCGAGCUCCAGAAAGGCAACGAGCUCGCCCUCCCCUCAAAAUACGUCAACUUCCUCUACCUCGCCUCACACUACGAGA |
| | | AACUCAAAGGCUCACCCGAGGACAACGAGCAGAAACAGCUCUUCGUCGAGCAGCACAAACACUACCUCGACGAGAUAAUA |
| | | GAGCAGAUAUCAGAGUUCUCAAAAAGGGUCAUACUCGCCGACGCCAACCUCGACAAAGUCCUCUCAGCCUACAACAAACA |
| | | CAGGGACAAACCCAUAAGGGAGCAGGCCGAGAACAUAAUACACCUCUUCACCCUCACCAACCUCGGCGCCCCCGCCGCCUU |
| | | CAAAUACUUCGACACCACCAUAGACAGGAAAAGGUACACCUCAACCAAAGAGGUCCUCGACGCCACCCUCAUACACCAGUC |
| | | AAUAACCGGCCUCUACGAGACCAGGAUAGACCUCUCACAGCUCGGCGGCGACGGCGGCGGCUCACCCAAAAAAAAAAGGA |
| | | AAGUCUAGCUAGCACCAGCCUCAAGAACACCCGAAUGGAGUCUCUAAGCUACAUAAUACCAACUUACACUUUACAAAAUG |
| | | UUGUCCCCCAAAAUGUAGCCAUUCGUAUCUGCUCCUAAUAAAAAGAAAGUUUCUUCACAUUCUCUCGAGAAAAAAAAAAA |
| | | AAAAAAAAAAAAAAAAAAAAAAAAAAAAAAAAAAAAAAAAAAAAAAAAAAAAAAAAAAAAAAAAAAAAAAAAAAAAAA |
| | | AAAAAAAAAAAUCUAG |
| 22 | Cas9 mRNA | GGGAAGCUCAGAAUAAACGCUCAACUUUGGCCGGAUCUGCCACCAUGGACAAAAAAUACAGCAUCGGCCUGGACAUCGGC |
| | transcript | ACGAACAGCGUGGGCUGGGCCGUGAUCACGGACGAGUACAAAGUGCCCAGCAAAAAAUUCAAAGUGCUGGGCAACACGGA |
| | comprising SEQ | CCGGCACAGCAUCAAAAAAAACCUGAUCGGCGCCCUGCUGUUCGACAGCGGCGAGACGGCCGAGGCCACGCGGCUGAAAC |
| | 12 | GGACGGCCCGGCGGCGGUACACGCGGCGGAAAAACCGGAUCUGCUACCUGCAGGAGAUCUUCAGCAACGAGAUGGCCAAA |
| | | GUGGACGACAGCUUCUUCCACCGGCUGGAGGAGAGCUUCCUGGUGGAGGAGGACAAAAAACACGAGCGGCACCCCAUCUU |
| | | CGGCAACAUCGUGGACGAGGUGGCCUACCACGAGAAAUACCCCACGAUCUACCACCUGCGGAAAAAACUGGUGGACAGCA |
| | | CGGACAAAGCCGACCUGCGGCUGAUCUACCUGGCCCUGGCCCACAUGAUCAAAUUCCGGGGCCACUUCCUGAUCGAGGGCG |
| | | ACCUGAACCCCGACAACAGCGACGUGGACAAACUGUUCAUCCAGCUGGUGCAGACGUACAACCAGCUGUUCGAGGAGAAC |
| | | CCCAUCAACGCCAGCGGCGUGGACGCCAAAGCCAUCCUGAGCGCCCGGCUGAGCAAAAGCCGGCGGCUGGAGAACCUGAUC |
| | | GCCCAGCUGCCCGGCGAGAAAAAAAACGGCCUGUUCGGCAACCUGAUCGCCCUGAGCCUGGGCCUGACGCCCAACUUCAAA |
| | | AGCAACUUCGACCUGGCCGAGGACGCCAAACUGCAGCUGAGCAAAGACACGUACGACGACGACCUGGACAACCUGCUGGC |
| | | CCAGAUCGGCGACCAGUACGCCGACCUGUUCCUGGCCGCCAAAAACCUGAGCGACGCCAUCCUGCUGAGCGACAUCCUGCG |
| | | GGUGAACACGGAGAUCACGAAAGCCCCCCUGAGCGCCAGCAUGAUCAAACGGUACGACGAGCACCACCAGGACCUGACGC |
| | | UGCUGAAAGCCCUGGUGCGGCAGCAGCUGCCCGAGAAAUACAAAGAGAUCUUCUUCGACCAGAGCAAAAACGGCUACGCC |
| | | GGCUACAUCGACGGCGGCGCCAGCCAGGAGGAGUUCUACAAAUUCAUCAAACCCAUCCUGGAGAAAAUGGACGGCACGGA |
| | | GGAGCUGCUGGUGAAACUGAACCGGGAGGACCUGCUGCGGAAACAGCGGACGUUCGACAACGGCAGCAUCCCCCACCAGA |
| | | UCCACCUGGGCGAGCUGCACGCCAUCCUGCGGCGGCAGGAGGACUUCUACCCCUUCCUGAAAGACAACCGGGAGAAAAUC |
| | | GAGAAAAUCCUGACGUUCCGGAUCCCCUACUACGUGGGCCCCCUGGCCCGGGGCAACAGCCGGUUCGCCUGGAUGACGCGG |
| | | AAAAGCGAGGAGACGAUCACGCCCUGGAACUUCGAGGAGGUGGUGGACAAAGGCGCCAGCGCCCAGAGCUUCAUCGAGCG |
| | | GAUGACGAACUUCGACAAAAACCUGCCCAACGAGAAAGUGCUGCCCAAACACAGCCUGCUGUACGAGUACUUCACGGUGU |
| | | ACAACGAGCUGACGAAAGUGAAAUACGUGACGGAGGGCAUGCGGAAACCCGCCUUCCUGAGCGGCGAGCAGAAAAAAGCC |
| | | AUCGUGGACCUGCUGUUCAAAACGAACCGGAAAGUGACGGUGAAACAGCUGAAAGAGGACUACUUCAAAAAAAUCGAGUG |
| | | CUUCGACAGCGUGGAGAUCAGCGGCGUGGAGGACCGGUUCAACGCCAGCCUGGGCACGUACCACGACCUGCUGAAAAUCA |
| | | UCAAAGACAAAGACUUCCUGGACAACGAGGAGAACGAGGACAUCCUGGAGGACAUCGUGCUGACGCUGACGCUGUUCGAG |
| | | GACCGGGAGAUGAUCGAGGAGCGGCUGAAAACGUACGCCCACCUGUUCGACGACAAAGUGAUGAAACAGCUGAAACGGCG |
| | | GCGGUACACGGGCUGGGGCCGGCUGAGCCGGAAACUGAUCAACGGCAUCCGGGACAAACAGAGCGGCAAAACGAUCCUGG |
| | | ACUUCCUGAAAAGCGACGGCUUCGCCAACCGGAACUUCAUGCAGCUGAUCCACGACGACAGCCUGACGUUCAAAGAGGAC |
| | | AUCCAGAAAGCCCAGGUGAGCGGCCAGGGCGACAGCCUGCACGAGCACAUCGCCAACCUGGCCGGCAGCCCCGCCAUCAAA |
| | | AAAGGCAUCCUGCAGACGGUGAAAGUGGUGGACGAGCUGGUGAAAGUGAUGGGCCGGCACAAACCCGAGAACAUCGUGAU |
| | | CGAGAUGGCCCGGGAGAACCAGACGACGCAGAAAGGCCAGAAAAACAGCCGGGAGCGGAUGAAACGGAUCGAGGAGGGCA |
| | | UCAAAGAGCUGGGCAGCCAGAUCCUGAAAGAGCACCCCGUGGAGAACACGCAGCUGCAGAACGAGAAACUGUACCUGUAC |
| | | UACCUGCAGAACGGCCGGGACAUGUACGUGGACCAGGAGCUGGACAUCAACCGGCUGAGCGACUACGACGUGGACCACAU |
| | | CGUGCCCCAGAGCUUCCUGAAAGACGACAGCAUCGACAACAAAGUGCUGACGCGGAGCGACAAAAACCGGGGCAAAAGCG |
| | | ACAACGUGCCCAGCGAGGAGGUGGUGAAAAAAAUGAAAAACUACUGGCGGCAGCUGCUGAACGCCAAACUGAUCACGCAG |
| | | CGGAAAUUCGACAACCUGACGAAAGCCGAGCGGGGCGGCCUGAGCGAGCUGGACAAAGCCGGCUUCAUCAAACGGCAGCU |
| | | GGUGGAGACGCGGCAGAUCACGAAACACGUGGCCCAGAUCCUGGACAGCCGGAUGAACACGAAAUACGACGAGAACGACA |
| | | AACUGAUCCGGGAGGUGAAAGUGAUCACGCUGAAAAGCAAACUGGUGAGCGACUUCCGGAAAGACUUCCAGUUCUACAAA |
| | | GUGCGGGAGAUCAACAACUACCACCACGCCCACGACGCCUACCUGAACGCCGUGGUGGGCACGGCCCUGAUCAAAAAAUAC |
| | | CCCAAACUGGAGAGCGAGUUCGUGUACGGCGACUACAAAGUGUACGACGUGCGGAAAAUGAUCGCCAAAAGCGAGCAGGA |
| | | GAUCGGCAAAGCCACGGCCAAAUACUUCUUCUACAGCAACAUCAUGAACUUCUUCAAAACGGAGAUCACGCUGGCCAACG |
| | | GCGAGAUCCGGAAACGGCCCCUGAUCGAGACGAACGGCGAGACGGGCGAGAUCGUGUGGGACAAAGGCCGGGACUUCGCC |
| | | ACGGUGCGGAAAGUGCUGAGCAUGCCCCAGGUGAACAUCGUGAAAAAAACGGAGGUGCAGACGGGCGGCUUCAGCAAAGA |
| | | GAGCAUCCUGCCCAAACGGAACAGCGACAAACUGAUCGCCCGGAAAAAAGACUGGGACCCCAAAAAAUACGGCGGCUUCG |
| | | ACAGCCCCACGGUGGCCUACAGCGUGCUGGUGGUGGCCAAAGUGGAGAAAGGCAAAAGCAAAAAACUGAAAAGCGUGAAA |
| | | GAGCUGCUGGGCAUCACGAUCAUGGAGCGGAGCAGCUUCGAGAAAAACCCCAUCGACUUCCUGGAGGCCAAAGGCUACAA |
| | | AGAGGUGAAAAAAGACCUGAUCAUCAAACUGCCCAAAUACAGCCUGUUCGAGCUGGAGAACGGCCGGAAACGGAUGCUGG |
| | | CCAGCGCCGGCGAGCUGCAGAAAGGCAACGAGCUGGCCCUGCCCAGCAAAUACGUGAACUUCCUGUACCUGGCCAGCCACU |
| | | ACGAGAAACUGAAAGGCAGCCCCGAGGACAACGAGCAGAAACAGCUGUUCGUGGAGCAGCACAAACACUACCUGGACGAG |
| | | AUCAUCGAGCAGAUCAGCGAGUUCAGCAAACGGGUGAUCCUGGCCGACGCCAACCUGGACAAAGUGCUGAGCGCCUACAA |
| | | CAAACACCGGGACAAACCCAUCCGGGAGCAGGCCGAGAACAUCAUCCACCUGUUCACGCUGACGAACCUGGGCGCCCCCGC |
| | | CGCCUUCAAAUACUUCGACACGACGAUCGACCGGAAACGGUACACGAGCACGAAAGAGGUGCUGGACGCCACGCUGAUCC |
| | | ACCAGAGCAUCACGGGCCUGUACGAGACGCGGAUCGACCUGAGCCAGCUGGGCGGCGACGGCGGCGGCAGCCCCAAAAAA |
| | | AAACGGAAAGUGUAGCUAGCACCAGCCUCAAGAACACCCGAAUGGAGUCUCUAAGCUACAUAAUACCAACUUACACUUUA |
| | | CAAAAUGUUGUCCCCCAAAAUGUAGCCAUUCGUAUCUGCUCCUAAUAAAAAGAAAGUUUCUUCACAUUCUCUCGAGAAAA |
| | | AAAAAAAAAAAAAAAAAAAAAAAAAAAAAAAAAAAAAAAAAAAAAAAAAAAAAAAAAAAAAAAAAAAAAAAAAAAAAA |
| | | AAAAAAAAAAAAAAAUCUAG |
| 23 | Cas9 transcript | GGGAAGCUCAGAAUAAACGCUCAACUUUGGCCGGAUCUGCCACCAUGGAUAAAAAAUAUUCAAUAGGAUUAGAUAUAGG |
| | comprising SEQ | AACAAAUUCAGUAGGAUGGGCAGUAAUAACAGAUGAAUAUAAAGUACCAUCAAAAAAAUUUAAAGUAUUAGGAAAUACA |
| | 13 | GAUAGACAUUCAAUAAAAAAAAAUUUAAUAGGAGCAUUAUUAUUUGAUUCAGGAGAAACAGCAGAAGCAACAAGAUUAA |
| | | AAAGAACAGCAAGAAGAAGAUAUACAAGAAGAAAAAAUAGAAUAUGUUAUUUACAAGAAAUAUUUUCAAAUGAAAUGGC |
| | | AAAAGUAGAUGAUUCAUUUUUUCAUAGAUUAGAAGAAUCAUUUUUAGUAGAAGAAGAUAAAAAACAUGAAAGACAUCCA |
| | | AUAUUUGGAAAUAUAGUAGAUGAAGUAGCAUAUCAUGAAAAAUAUCCAACAAUAUAUCAUUUAAGAAAAAAAUUAGUAG |
| | | AUUCAACAGAUAAAGCAGAUUUAAGAUUAAUAUAUUUAGCAUUAGCACAUAUGAUAAAAUUUAGAGGACAUUUUUUAAU |
| | | AGAAGGAGAUUUAAAUCCAGAUAAUUCAGAUGUAGAUAAAUUAUUUAUACAAUUAGUACAAACAUAUAAUCAAUUAUUU |
| | | GAAGAAAAUCCAAUAAAUGCAUCAGGAGUAGAUGCAAAAGCAAUAUUAUCAGCAAGAUUAUCAAAAUCAAGAAGAUUAG |
| | | AAAAUUUAAUAGCACAAUUACCAGGAGAAAAAAAAAAUGGAUUAUUUGGAAAUUUAAUAGCAUUAUCAUUAGGAUUAAC |
| | | ACCAAAUUUUAAAUCAAAUUUUGAUUUAGCAGAAGAUGCAAAAUUACAAUUAUCAAAAGAUACAUAUGAUGAUGAUUUA |
| | | GAUAAUUUAUUAGCACAAAUAGGAGAUCAAUAUGCAGAUUUAUUUUUAGCAGCAAAAAAUUUAUCAGAUGCAAUAUUAU |
| | | UAUCAGAUAUAUUAAGAGUAAAUACAGAAAUAACAAAAGCACCAUUAUCAGCAUCAAUGAUAAAAAGAUAUGAUGAACA |
| | | UCAUCAGGACUUAACAUUAUUAAAAGCAUUAGUAAGACAACAAUUACCAGAAAAAUAUAAAGAAAUAUUUUUUGAUCAA |
| | | UCAAAAAAUGGAUAUGCAGGAUAUAUAGAUGGAGGAGCAUCACAAGAAGAAUUUUAUAAAUUUAUAAAACCAAUAUUAG |
| | | AAAAAAUGGAUGGAACAGAAGAAUUAUUAGUAAAAUUAAAUAGAGAAGAUUUAUUAAGAAAACAAAGAACAUUUGAUAA |
| | | UGGAUCAAUACCACAUCAAAUACAUUUAGGAGAAUUACAUGCAAUAUUAAGAAGACAAGAAGAUUUUUAUCCAUUUUUA |
| | | AAAGAUAAUAGAGAAAAAAUAGAAAAAAUAUUAACAUUUAGAAUACCAUAUUAUGUAGGACCAUUAGCAAGAGGAAAUU |
| | | CAAGAUUUGCAUGGAUGACAAGAAAAUCAGAAGAAACAAUAACACCAUGGAAUUUUGAAGAAGUAGUAGAUAAAGGAGC |
| | | AUCAGCACAAUCAUUUAUAGAAAGAAUGACAAAUUUUGAUAAAAAUUUACCAAAUGAAAAAGUAUUACCAAAACAUUCA |
| | | UUAUUAUAUGAAUAUUUUACAGUAUAUAAUGAAUUAACAAAAGUAAAAUAUGUAACAGAAGGAAUGAGAAAACCAGCAU |
| | | UUUUAUCAGGAGAACAAAAAAAAGCAAUAGUAGAUUUAUUAUUUAAAACAAAUAGAAAAGUAACAGUAAAACAAUUAAA |
| | | AGAAGAUUAUUUUAAAAAAAUAGAAUGUUUUGAUUCAGUAGAAAUAUCAGGAGUAGAAGAUAGAUUUAAUGCAUCAUUA |
| | | GGAACAUAUCAUGAUUUAUUAAAAAUAAUAAAAGAUAAAGAUUUUUUAGAUAAUGAAGAAAAUGAAGAUAUAUUAGAAG |
| | | AUAUAGUAUUAACAUUAACAUUAUUUGAAGAUAGAGAAAUGAUAGAAGAAAGAUUAAAAACAUAUGCACAUUUAUUUGA |
| | | UGAUAAAGUAAUGAAACAAUUAAAAAGAAGAAGAUAUACAGGAUGGGGAAGAUUAUCAAGAAAAUUAAUAAAUGGAAUA |
| | | AGAGAUAAACAAUCAGGAAAAACAAUAUUAGAUUUUUUAAAAUCAGAUGGAUUUGCAAAUAGAAAUUUUAUGCAAUUAA |
| | | UACAUGAUGAUUCAUUAACAUUUAAAGAAGAUAUACAAAAAGCACAAGUAUCAGGACAAGGAGAUUCAUUACAUGAACA |
| | | UAUAGCAAAUUUAGCAGGAUCACCAGCAAUAAAAAAAGGAAUAUUACAAACAGUAAAAGUAGUAGAUGAAUUAGUAAAA |
| | | GUAAUGGGAAGACAUAAACCAGAAAAUAUAGUAAUAGAAAUGGCAAGAGAAAAUCAAACAACACAAAAAGGACAAAAAA |
| | | AUUCAAGAGAAAGAAUGAAAAGAAUAGAAGAAGGAAUAAAAGAAUUAGGAUCACAAAUAUUAAAAGAACAUCCAGUAGA |
| | | AAAUACACAAUUACAAAAUGAAAAAUUAUAUUUAUAUUAUUUACAAAAUGGAAGAGAUAUGUAUGUAGAUCAAGAAUUA |
| | | GAUAUAAAUAGAUUAUCAGAUUAUGAUGUAGAUCAUAUAGUACCACAAUCAUUUUUAAAAGAUGAUUCAAUAGAUAAUA |
| | | AAGUAUUAACAAGAUCAGAUAAAAAUAGAGGAAAAUCAGAUAAUGUACCAUCAGAAGAAGUAGUAAAAAAAAUGAAAAA |
| | | UUAUUGGAGACAAUUAUUAAAUGCAAAAUUAAUAACACAAAGAAAAUUUGAUAAUUUAACAAAAGCAGAAAGAGGAGGA |
| | | UUAUCAGAAUUAGAUAAAGCAGGAUUUAUAAAAAGACAAUUAGUAGAAACAAGACAAAUAACAAAACAUGUAGCACAAA |
| | | UAUUAGAUUCAAGAAUGAAUACAAAAUAUGAUGAAAAUGAUAAAUUAAUAAGAGAAGUAAAAGUAAUAACAUUAAAAUC |
| | | AAAAUUAGUAUCAGAUUUUAGAAAAGAUUUUCAAUUUUAUAAAGUAAGAGAAAUAAAUAAUUAUCAUCAUGCACAUGAU |
| | | GCAUAUUUAAAUGCAGUAGUAGGAACAGCAUUAAUAAAAAAAUAUCCAAAAUUAGAAUCAGAAUUUGUAUAUGGAGAUU |
| | | AUAAAGUAUAUGAUGUAAGAAAAAUGAUAGCAAAAUCAGAACAAGAAAUAGGAAAAGCAACAGCAAAAUAUUUUUUUUA |
| | | UUCAAAUAUAAUGAAUUUUUUUAAAACAGAAAUAACAUUAGCAAAUGGAGAAAUAAGAAAAAGACCAUUAAUAGAAACA |
| | | AAUGGAGAAACAGGAGAAAUAGUAUGGGAUAAAGGAAGAGAUUUUGCAACAGUAAGAAAAGUAUUAUCAAUGCCACAAG |
| | | UAAAUAUAGUAAAAAAAACAGAAGUACAAACAGGAGGAUUUUCAAAAGAAUCAAUAUUACCAAAAAGAAAUUCAGAUAA |
| | | AUUAAUAGCAAGAAAAAAAGAUUGGGAUCCAAAAAAAUAUGGAGGAUUUGAUUCACCAACAGUAGCAUAUUCAGUAUUA |
| | | GUAGUAGCAAAAGUAGAAAAAGGAAAAUCAAAAAAAUUAAAAUCAGUAAAAGAAUUAUUAGGAAUAACAAUAAUGGAAA |
| | | GAUCAUCAUUUGAAAAAAAUCCAAUAGAUUUUUUAGAAGCAAAAGGAUAUAAAGAAGUAAAAAAAGAUUUAAUAAUAAA |
| | | AUUACCAAAAUAUUCAUUAUUUGAAUUAGAAAAUGGAAGAAAAAGAAUGUUAGCAUCAGCAGGAGAAUUACAAAAAGGA |
| | | AAUGAAUUAGCAUUACCAUCAAAAUAUGUAAAUUUUUUAUAUUUAGCAUCACAUUAUGAAAAAUUAAAAGGAUCACCAG |
| | | AAGAUAAUGAACAAAAACAAUUAUUUGUAGAACAACAUAAACAUUAUUUAGAUGAAAUAAUAGAACAAAUAUCAGAAUU |
| | | UUCAAAAAGAGUAAUAUUAGCAGAUGCAAAUUUAGAUAAAGUAUUAUCAGCAUAUAAUAAACAUAGAGAUAAACCAAUA |
| | | AGAGAACAAGCAGAAAAUAUAAUACAUUUAUUUACAUUAACAAAUUUAGGAGCACCAGCAGCAUUUAAAUAUUUUGAUA |
| | | CAACAAUAGAUAGAAAAAGAUAUACAUCAACAAAAGAAGUAUUAGAUGCAACAUUAAUACAUCAAUCAAUAACAGGAUU |
| | | AUAUGAAACAAGAAUAGAUUUAUCACAAUUAGGAGGAGAUGGAGGAGGAUCACCAAAAAAAAAAAGAAAAGUAUAGCUA |
| | | GCACCAGCCUCAAGAACACCCGAAUGGAGUCUCUAAGCUACAUAAUACCAACUUACACUUUACAAAAUGUUGUCCCCCAA |
| | | AAUGUAGCCAUUCGUAUCUGCUCCUAAUAAAAAGAAAGUUUCUUCACAUUCUCUCGAGAAAAAAAAAAAAAAAAAAAAA |
| | | AAAAAAAAAAAAAAAAAAAAAAAAAAAAAAAAAAAAAAAAAAAAAAAAAAAAAAAAAAAAAAAAAAAAAAAAAUCUAG |
| 24 | Cas9 transcript | GGGAAGCUCAGAAUAAACGCUCAACUUUGGCCGGAUCUGCCACCAUGGAUAAAAAGUACAGCAUCGGAUUAGAUAUAGGA |
| | comprising SEQ | ACAAAUUCAGUUGGCUGGGCUGUGAUAACAGAUGAAUAUAAAGUUCCCUCAAAAAAAUUUAAAGUAUUAGGAAAUACAG |
| | 14 | AUAGACAUAGCAUCAAAAAAAAUCUCAUAGGUGCACUGUUAUUUGAUUCAGGUGAGACAGCAGAAGCCACAAGAUUAAA |
| | | AAGAACAGCCCGCAGAAGAUAUACAAGAAGAAAAAAUAGAAUAUGUUAUUUACAGGAGAUAUUUUCAAAUGAAAUGGCA |
| | | AAAGUAGAUGAUUCAUUUUUUCAUAGAUUAGAAGAAUCAUUCCUGGUAGAAGAAGAUAAAAAACAUGAAAGACAUCCAA |
| | | UAUUUGGAAAUAUAGUAGAUGAAGUCGCAUAUCAUGAAAAGUACCCCACCAUAUAUCAUCUGCGGAAAAAAUUAGUAGA |
| | | UUCGACUGAUAAAGCAGAUCUGCGGUUAAUAUAUUUAGCACUGGCACAUAUGAUAAAAUUUAGAGGACAUUUCCUGAUA |
| | | GAAGGAGAUUUAAAUCCUGACAAUUCAGAUGUAGAUAAAUUAUUUAUACAAUUAGUACAAACCUACAAUCAAUUAUUUG |
| | | AAGAAAAUCCAAUAAAUGCAUCAGGAGUAGAUGCAAAAGCAAUACUCAGCGCCCGCCUCAGCAAAUCAAGAAGAUUAGAA |
| | | AAUCUCAUAGCACAACUUCCAGGUGAGAAAAAAAAUGGGUUAUUUGGAAAUCUCAUAGCACUCAGCUUAGGAUUAACUCC |
| | | CAAUUUUAAAUCAAAUUUUGAUUUAGCAGAAGAUGCAAAAUUACAACUCAGCAAAGAUACCUACGAUGAUGAUUUAGAU |
| | | AAUCUCUUAGCACAAAUAGGAGAUCAAUAUGCAGAUUUAUUCCUGGCUGCCAAAAAUCUCAGCGAUGCAAUAUUACUCAG |
| | | CGAUAUACUGCGGGUAAAUACAGAGAUAACAAAAGCACCACUCAGCGCAUCAAUGAUAAAAAGAUAUGAUGAACAUCAUC |
| | | AAGAUUUAACAUUAUUAAAAGCACUGGUAAGACAACAACUUCCAGAGAAGUACAAAGAAAUAUUUUUUGAUCAGAGCAA |
| | | AAAUGGGUAUGCCGGGUAUAUAGAUGGUGGUGCCUCACAGGAGGAAUUUUAUAAAUUUAUAAAACCAAUAUUAGAAAAA |
| | | AUGGAUGGAACAGAGGAGCUGUUAGUAAAAUUAAAUAGGGAGGAUUUACUGCGGAAACAAAGAACAUUUGAUAAUGGGA |
| | | GCAUCCCCCAUCAAAUACAUUUAGGUGAGCUGCAUGCAAUACUGCGGAGACAGGAGGAUUUUUAUCCAUUCCUGAAAGAU |
| | | AAUAGGGAGAAAAUAGAAAAAAUAUUAACAUUUAGAAUCCCCUAUUAUGUUGGCCCAUUAGCCCGCGGAAAUUCAAGAU |
| | | UUGCAUGGAUGACAAGAAAAUCAGAAGAAACAAUAACUCCCUGGAAUUUUGAAGAAGUCGUAGAUAAGGGUGCCUCAGC |
| | | ACAGAGCUUUAUAGAAAGAAUGACAAAUUUUGAUAAAAAUCUUCCAAAUGAAAAAGUACUUCCAAAACAUUCAUUAUUA |
| | | UAUGAAUAUUUUACAGUAUAUAAUGAGCUGACAAAAGUAAAGUACGUAACAGAGGGAAUGAGAAAACCAGCAUUCCUCA |
| | | GCGGUGAGCAAAAAAAAGCAAUAGUAGAUUUAUUAUUUAAAACAAAUAGAAAAGUAACAGUAAAACAAUUAAAAGAAGA |
| | | UUAUUUUAAAAAAAUAGAAUGUUUUGAUUCAGUAGAAAUAUCAGGAGUAGAAGAUAGAUUUAAUGCAUCAUUAGGAACC |
| | | UACCAUGAUUUAUUAAAAAUAAUAAAAGAUAAAGAUUUCCUGGAUAAUGAAGAAAAUGAAGAUAUAUUAGAAGAUAUAG |
| | | UAUUAACAUUAACAUUAUUUGAAGAUAGGGAGAUGAUAGAAGAAAGAUUAAAAACCUACGCACAUUUAUUUGAUGAUAA |
| | | AGUAAUGAAACAAUUAAAAAGAAGAAGAUAUACAGGAUGGGGAAGACUCAGCAGAAAAUUAAUAAAUGGGAUACGAGAC |
| | | AAACAGAGCGGAAAAACAAUAUUAGAUUUCCUGAAAUCAGAUGGAUUUGCAAAUAGAAAUUUUAUGCAAUUAAUACAUG |
| | | AUGAUUCAUUAACAUUUAAAGAAGAUAUACAAAAAGCACAGGUCAGCGGACAGGGCGAUUCAUUACAUGAACAUAUAGC |
| | | AAAUCUCGCCGGGUCACCAGCAAUAAAAAAGGGGAUAUUACAAACAGUAAAAGUAGUAGAUGAGCUGGUAAAAGUAAUG |
| | | GGAAGACAUAAACCAGAGAAUAUAGUAAUAGAAAUGGCCAGGGAGAAUCAAACAACUCAAAAGGGGCAAAAAAAUUCAA |
| | | GGGAGAGAAUGAAAAGAAUAGAAGAAGGAAUAAAAGAGCUGGGAUCACAAAUAUUAAAAGAACAUCCAGUAGAAAAUAC |
| | | UCAAUUACAAAAUGAAAAAUUAUAUUUAUAUUAUUUACAAAAUGGGCGAGACAUGUAUGUAGAUCAGGAGCUGGAUAUA |
| | | AAUAGACUCAGCGAUUAUGAUGUAGAUCAUAUAGUUCCCCAGAGCUUCCUGAAAGAUGAUAGCAUCGAUAAUAAAGUAU |
| | | UAACAAGAUCAGAUAAAAAUAGAGGAAAAUCAGAUAAUGUUCCCUCAGAAGAAGUCGUAAAAAAAAUGAAAAAUUAUUG |
| | | GAGACAAUUAUUAAAUGCAAAAUUAAUAACUCAAAGAAAAUUUGAUAAUCUCACAAAAGCAGAAAGAGGUGGCCUCAGC |
| | | GAGCUGGAUAAAGCCGGGUUUAUAAAAAGACAAUUAGUAGAAACAAGACAAAUAACAAAACAUGUAGCACAAAUAUUAG |
| | | AUUCAAGAAUGAAUACAAAGUACGAUGAAAAUGAUAAAUUAAUAAGGGAAGUCAAAGUAAUAACAUUAAAAUCAAAAUU |
| | | AGUCAGCGAUUUUAGAAAAGAUUUUCAAUUUUAUAAAGUAAGGGAGAUAAAUAAUUAUCAUCAUGCACAUGAUGCAUAU |
| | | UUAAAUGCUGUGGUUGGCACAGCACUGAUAAAAAAGUACCCAAAAUUAGAAUCAGAAUUUGUAUAUGGAGAUUAUAAAG |
| | | UAUAUGAUGUAAGAAAAAUGAUAGCAAAAUCAGAACAGGAGAUAGGAAAAGCAACAGCAAAGUACUUUUUUUAUUCAAA |
| | | UAUAAUGAAUUUUUUUAAAACAGAGAUAACAUUAGCAAAUGGUGAGAUAAGAAAAAGACCAUUAAUAGAAACAAAUGGU |
| | | GAGACAGGUGAGAUAGUAUGGGAUAAGGGGCGAGACUUUGCAACAGUAAGAAAAGUACUCAGCAUGCCACAGGUGAAUA |
| | | UAGUAAAAAAAACAGAAGUCCAAACAGGUGGCUUUUCAAAAGAAAGCAUCCUUCCAAAAAGAAAUUCAGAUAAAUUAAU |
| | | AGCCCGCAAAAAAGAUUGGGAUCCAAAAAAGUACGGUGGCUUUGAUUCACCCACCGUAGCAUAUUCAGUAUUAGUAGUAG |
| | | CAAAAGUAGAAAAGGGGAAAUCAAAAAAAUUAAAAUCAGUAAAAGAGCUGUUAGGAAUAACAAUAAUGGAAAGAUCAUC |
| | | AUUUGAAAAAAAUCCAAUAGAUUUCCUGGAAGCCAAGGGGUAUAAAGAAGUCAAAAAAGAUUUAAUAAUAAAACUUCCA |
| | | AAGUACUCAUUAUUUGAGCUGGAAAAUGGGAGAAAAAGAAUGUUAGCAUCAGCCGGUGAGCUGCAAAAGGGGAAUGAGC |
| | | UGGCACUUCCCUCAAAGUACGUAAAUUUCCUGUAUUUAGCAUCACAUUAUGAAAAAUUAAAGGGGUCACCAGAGGAUAAU |
| | | GAACAAAAACAAUUAUUUGUAGAACAACAUAAACAUUAUUUAGAUGAAAUAAUAGAACAAAUAUCAGAAUUUUCAAAAA |
| | | GAGUAAUAUUAGCAGAUGCAAAUCUCGAUAAAGUACUCAGCGCAUAUAAUAAACAUCGAGACAAACCAAUAAGGGAGCA |
| | | GGCCGAAAAUAUAAUACAUUUAUUUACAUUAACAAAUCUCGGUGCCCCAGCUGCCUUUAAGUACUUUGAUACAACAAUAG |
| | | AUAGAAAAAGAUAUACAUCGACUAAAGAAGUCUUAGAUGCAACAUUAAUACAUCAGAGCAUCACAGGAUUAUAUGAAAC |
| | | AAGAAUAGAUCUCAGCCAAUUAGGUGGCGAUGGUGGUGGCUCACCAAAAAAAAAAAGAAAAGUAUAGCUAGCACCAGCCU |
| | | CAAGAACACCCGAAUGGAGUCUCUAAGCUACAUAAUACCAACUUACACUUUACAAAAUGUUGUCCCCCAAAAUGUAGCCA |
| | | UUCGUAUCUGCUCCUAAUAAAAAGAAAGUUUCUUCACAUUCUCUCGAGAAAAAAAAAAAAAAAAAAAAAAAAAAAAAAA |
| | | AAAAAAAAAAAAAAAAAAAAAAAAAAAAAAAAAAAAAAAAAAAAAAAAAAAAAAAAAAAAAAAAUCUAG |
| 25-28 | | Not Used |
| 29 | E-pair enriched, | AUGGACAAGAAGUACAGCAUCGGACUGGACAUCGGAACAAACAGCGUUGGCUGGGCUGUGAUCACAGACGAAUACAAGGU |
| | Table 6 codon | UCCCUCAAAGAAGUUCAAGGUCCUGGGAAACACAGACAGACACAGCAUCAAGAAGAAUCUCAUCGGUGCACUGCUGUUCG |
| | enriched Cas9 | ACAGCGGUGAGACAGCAGAAGCCACAAGACUGAAGAGAACAGCCCGCAGAAGAUACACAAGAAGAAAGAACAGAAUCUGC |
| | ORF | UACCUGCAGGAGAUCUUCAGCAACGAAAUGGCAAAGGUCGACGACAGCUUCUUCCACAGACUGGAAGAAAGCUUCCUGGU |
| | | CGAAGAAGACAAGAAGCACGAAAGACACCCGAUCUUCGGAAACAUCGUCGACGAAGUCGCAUACCACGAAAAGUACCCCA |
| | | CCAUCUACCACCUGCGGAAGAAGCUGGUCGACUCGACUGACAAGGCAGACCUGCGGCUGAUCUACCUGGCACUGGCACAC |
| | | AUGAUAAAGUUCAGAGGACACUUCCUGAUCGAAGGAGACCUGAACCCUGACAACAGCGACGUCGACAAGCUGUUCAUCCA |
| | | GCUGGUCCAGACCUACAACCAGCUGUUCGAAGAAAACCCGAUCAACGCAAGCGGAGUCGACGCAAAGGCAAUCCUCAGCG |
| | | CCCGCCUCAGCAAGAGCAGAAGACUGGAAAAUCUCAUCGCACAGCUUCCAGGUGAGAAGAAGAAUGGGCUGUUCGGAAAU |
| | | CUCAUCGCACUCAGCCUGGGACUGACUCCCAACUUCAAGAGCAACUUCGACCUGGCAGAAGACGCAAAGCUGCAGCUCAGC |
| | | AAGGACACCUACGACGACGACCUGGACAAUCUCCUGGCACAGAUCGGAGACCAGUACGCAGACCUGUUCCUGGCUGCCAA |
| | | GAAUCUCAGCGACGCAAUCCUGCUCAGCGACAUCCUGCGGGUCAACACAGAGAUCACAAAGGCACCGCUCAGCGCAAGCA |
| | | UGAUAAAGAGAUACGACGAACACCACCAGGACCUGACACUGCUGAAGGCACUGGUCAGACAGCAGCUUCCAGAGAAGUAC |
| | | AAGGAAAUCUUCUUCGACCAGAGCAAGAAUGGGUACGCCGGGUACAUCGACGGUGGUGCCAGCCAGGAGGAAUUCUACAA |
| | | GUUCAUCAAGCCGAUCCUGGAAAAGAUGGACGGAACAGAGGAGCUGCUGGUCAAGCUGAACAGGGAGGACCUGCUGCGGA |
| | | AGCAGAGAACAUUCGACAAUGGGAGCAUCCCCCACCAGAUCCACCUGGGUGAGCUGCACGCAAUCCUGCGGAGACAGGAG |
| | | GACUUCUACCCGUUCCUGAAGGACAACAGGGAGAAGAUCGAAAAGAUCCUGACAUUCAGAAUCCCCUACUACGUUGGCCC |
| | | GCUGGCCCGCGGAAACAGCAGAUUCGCAUGGAUGACAAGAAAGAGCGAAGAAACAAUCACUCCCUGGAACUUCGAAGAAG |
| | | UCGUCGACAAGGGUGCCAGCGCACAGAGCUUCAUCGAAAGAAUGACAAACUUCGACAAGAAUCUUCCAAACGAAAAGGUC |
| | | CUUCCAAAGCACAGCCUGCUGUACGAAUACUUCACAGUCUACAACGAGCUGACAAAGGUCAAGUACGUCACAGAGGGAAU |
| | | GAGAAAGCCGGCAUUCCUCAGCGGUGAGCAGAAGAAGGCAAUCGUCGACCUGCUGUUCAAGACAAACAGAAAGGUCACAG |
| | | UCAAGCAGCUGAAGGAAGACUACUUCAAGAAGAUCGAAUGCUUCGACAGCGUCGAAAUCAGCGGAGUCGAAGACAGAUUC |
| | | AACGCAAGCCUGGGAACCUACCACGACCUGCUGAAGAUCAUCAAGGACAAGGACUUCCUGGACAACGAAGAAAACGAAGA |
| | | CAUCCUGGAAGACAUCGUCCUGACACUGACACUGUUCGAAGACAGGGAGAUGAUAGAAGAAAGACUGAAGACCUACGCAC |
| | | ACCUGUUCGACGACAAGGUCAUGAAGCAGCUGAAGAGAAGAAGAUACACAGGAUGGGGAAGACUCAGCAGAAAGCUGAUC |
| | | AAUGGGAUCCGAGACAAGCAGAGCGGAAAGACAAUCCUGGACUUCCUGAAGAGCGACGGAUUCGCAAACAGAAACUUCAU |
| | | GCAGCUGAUCCACGACGACAGCCUGACAUUCAAGGAAGACAUCCAGAAGGCACAGGUCAGCGGACAGGGCGACAGCCUGC |
| | | ACGAACACAUCGCAAAUCUCGCCGGGAGCCCGGCAAUCAAGAAGGGGAUCCUGCAGACAGUCAAGGUCGUCGACGAGCUG |
| | | GUCAAGGUCAUGGGAAGACACAAGCCAGAGAACAUCGUCAUCGAAAUGGCCAGGGAGAACCAGACAACUCAAAAGGGGCA |
| | | GAAGAACAGCAGGGAGAGAAUGAAGAGAAUCGAAGAAGGAAUCAAGGAGCUGGGAAGCCAGAUCCUGAAGGAACACCCG |
| | | GUCGAAAACACUCAACUGCAGAACGAAAAGCUGUACCUGUACUACCUGCAGAAUGGGCGAGACAUGUACGUCGACCAGGA |
| | | GCUGGACAUCAACAGACUCAGCGACUACGACGUCGACCACAUCGUUCCCCAGAGCUUCCUGAAGGACGACAGCAUCGACA |
| | | ACAAGGUCCUGACAAGAAGCGACAAGAACAGAGGAAAGAGCGACAACGUUCCCUCAGAAGAAGUCGUCAAGAAGAUGAAG |
| | | AACUACUGGAGACAGCUGCUGAACGCAAAGCUGAUCACUCAAAGAAAGUUCGACAAUCUCACAAAGGCAGAAAGAGGUGG |
| | | CCUCAGCGAGCUGGACAAGGCCGGGUUCAUCAAGAGACAGCUGGUCGAAACAAGACAGAUCACAAAGCACGUCGCACAGA |
| | | UCCUGGACAGCAGAAUGAACACAAAGUACGACGAAAACGACAAGCUGAUCAGGGAAGUCAAGGUCAUCACACUGAAGAGC |
| | | AAGCUGGUCAGCGACUUCAGAAAGGACUUCCAGUUCUACAAGGUCAGGGAGAUCAACAACUACCACCACGCACACGACGC |
| | | AUACCUGAACGCUGUGGUUGGCACAGCACUGAUCAAGAAGUACCCGAAGCUGGAAAGCGAAUUCGUCUACGGAGACUACA |
| | | AGGUCUACGACGUCAGAAAGAUGAUAGCAAAGAGCGAACAGGAGAUCGGAAAGGCAACAGCAAAGUACUUCUUCUACAGC |
| | | AACAUCAUGAACUUCUUCAAGACAGAGAUCACACUGGCAAAUGGUGAGAUCAGAAAGAGACCGCUGAUCGAAACAAAUGG |
| | | UGAGACAGGUGAGAUCGUCUGGGACAAGGGGCGAGACUUCGCAACAGUCAGAAAGGUCCUCAGCAUGCCGCAGGUGAACA |
| | | UCGUCAAGAAGACAGAAGUCCAGACAGGUGGCUUCAGCAAGGAAAGCAUCCUUCCAAAGAGAAACAGCGACAAGCUGAUC |
| | | GCCCGCAAGAAGGACUGGGACCCGAAGAAGUACGGUGGCUUCGACAGCCCCACCGUCGCAUACAGCGUCCUGGUCGUCGC |
| | | AAAGGUCGAAAAGGGGAAGAGCAAGAAGCUGAAGAGCGUCAAGGAGCUGCUGGGAAUCACAAUCAUGGAAAGAAGCAGC |
| | | UUCGAAAAGAACCCGAUCGACUUCCUGGAAGCCAAGGGGUACAAGGAAGUCAAGAAGGACCUGAUCAUCAAGCUUCCAAA |
| | | GUACAGCCUGUUCGAGCUGGAAAAUGGGAGAAAGAGAAUGCUGGCAAGCGCCGGUGAGCUGCAGAAGGGGAACGAGCUG |
| | | GCACUUCCCUCAAAGUACGUCAACUUCCUGUACCUGGCAAGCCACUACGAAAAGCUGAAGGGGAGCCCAGAGGACAACGA |
| | | ACAGAAGCAGCUGUUCGUCGAACAGCACAAGCACUACCUGGACGAAAUCAUCGAACAGAUCAGCGAAUUCAGCAAGAGAG |
| | | UCAUCCUGGCAGACGCAAAUCUCGACAAGGUCCUCAGCGCAUACAACAAGCACCGAGACAAGCCGAUCAGGGAGCAGGCC |
| | | GAAAACAUCAUCCACCUGUUCACACUGACAAAUCUCGGUGCCCCGGCUGCCUUCAAGUACUUCGACACAACAAUCGACAG |
| | | AAAGAGAUACACAUCGACUAAGGAAGUCCUGGACGCAACACUGAUCCACCAGAGCAUCACAGGACUGUACGAAACAAGAA |
| | | UCGACCUCAGCCAGCUGGGUGGCGACGGUGGUGGCAGCCCGAAGAAGAAGAGAAAGGUCUAG |
| 30-45 | | Not Used |
| 46 | E-pair enriched, | AUGGACAAGAAGUACAGCAUCGGCCUGGACAUCGGCACCAACUCCGUUGGCUGGGCUGUGAUCACCGACGAGUACAAGGU |
| | Table 7 Low A | UCCCUCAAAGAAGUUCAAGGUGCUGGGCAACACCGACCGGCACAGCAUCAAGAAGAAUCUCAUCGGUGCACUGCUGUUCG |
| | codon enriched | ACUCCGGUGAGACCGCCGAAGCCACCCGGCUGAAGCGGACCGCCCGCCGGCGGUACACCCGGCGGAAGAACCGGAUCUGCU |
| | Cas9 ORF | ACCUGCAGGAGAUCUUCUCCAACGAGAUGGCCAAGGUGGACGACUCCUUCUUCCACCGGCUGGAGGAGUCCUUCCUGGUG |
| | | GAGGAGGACAAGAAGCACGAGCGGCACCCCAUCUUCGGCAACAUCGUGGACGAAGUCGCCUACCACGAGAAGUACCCCAC |
| | | CAUCUACCACCUGCGGAAGAAGCUGGUGGACUCGACUGACAAGGCCGACCUGCGGCUGAUCUACCUGGCACUGGCCCACA |
| | | UGAUAAAGUUCCGGGGCCACUUCCUGAUCGAGGGCGACCUGAACCCUGACAACUCCGACGUGGACAAGCUGUUCAUCCAG |
| | | CUGGUGCAGACCUACAACCAGCUGUUCGAGGAGAACCCCAUCAACGCCUCCGGCGUGGACGCCAAGGCCAUCCUCAGCGCC |
| | | CGCCUCAGCAAGUCCCGGCGGCUGGAGAAUCUCAUCGCCCAGCUUCCAGGUGAGAAGAAGAAUGGGCUGUUCGGCAAUCU |
| | | CAUCGCACUCAGCCUGGGCCUGACUCCCAACUUCAAGUCCAACUUCGACCUGGCCGAGGACGCCAAGCUGCAGCUCAGCAA |
| | | GGACACCUACGACGACGACCUGGACAAUCUCCUGGCCCAGAUCGGCGACCAGUACGCCGACCUGUUCCUGGCUGCCAAGAA |
| | | UCUCAGCGACGCCAUCCUGCUCAGCGACAUCCUGCGGGUGAACACAGAGAUCACCAAGGCCCCCCUCAGCGCCUCCAUGAU |
| | | AAAGCGGUACGACGAGCACCACCAGGACCUGACCCUGCUGAAGGCACUGGUGCGGCAGCAGCUUCCAGAGAAGUACAAGG |
| | | AGAUCUUCUUCGACCAGAGCAAGAAUGGGUACGCCGGGUACAUCGACGGUGGUGCCUCCCAGGAGGAGUUCUACAAGUUC |
| | | AUCAAGCCCAUCCUGGAGAAGAUGGACGGCACAGAGGAGCUGCUGGUGAAGCUGAACAGGGAGGACCUGCUGCGGAAGCA |
| | | GCGGACCUUCGACAAUGGGAGCAUCCCCCACCAGAUCCACCUGGGUGAGCUGCACGCCAUCCUGCGGCGGCAGGAGGACUU |
| | | CUACCCCUUCCUGAAGGACAACAGGGAGAAGAUCGAGAAGAUCCUGACCUUCCGGAUCCCCUACUACGUUGGCCCCCUGGC |
| | | CCGCGGCAACUCCCGGUUCGCCUGGAUGACCCGGAAGUCCGAGGAGACCAUCACUCCCUGGAACUUCGAGGAAGUCGUGG |
| | | ACAAGGGUGCCUCCGCCCAGAGCUUCAUCGAGCGGAUGACCAACUUCGACAAGAAUCUUCCAAACGAGAAGGUGCUUCCA |
| | | AAGCACUCCCUGCUGUACGAGUACUUCACCGUGUACAACGAGCUGACCAAGGUGAAGUACGUGACAGAGGGCAUGCGGAA |
| | | GCCCGCCUUCCUCAGCGGUGAGCAGAAGAAGGCCAUCGUGGACCUGCUGUUCAAGACCAACCGGAAGGUGACCGUGAAGC |
| | | AGCUGAAGGAGGACUACUUCAAGAAGAUCGAGUGCUUCGACUCCGUGGAGAUCUCCGGCGUGGAGGACCGGUUCAACGCC |
| | | UCCCUGGGCACCUACCACGACCUGCUGAAGAUCAUCAAGGACAAGGACUUCCUGGACAACGAGGAGAACGAGGACAUCCU |
| | | GGAGGACAUCGUGCUGACCCUGACCCUGUUCGAGGACAGGGAGAUGAUAGAGGAGCGGCUGAAGACCUACGCCCACCUGU |
| | | UCGACGACAAGGUGAUGAAGCAGCUGAAGCGGCGGCGGUACACCGGCUGGGGCCGGCUCAGCCGGAAGCUGAUCAAUGGG |
| | | AUCCGAGACAAGCAGAGCGGCAAGACCAUCCUGGACUUCCUGAAGUCCGACGGCUUCGCCAACCGGAACUUCAUGCAGCU |
| | | GAUCCACGACGACUCCCUGACCUUCAAGGAGGACAUCCAGAAGGCCCAGGUCAGCGGCCAGGGCGACUCCCUGCACGAGCA |
| | | CAUCGCCAAUCUCGCCGGGUCCCCCGCCAUCAAGAAGGGGAUCCUGCAGACCGUGAAGGUGGUGGACGAGCUGGUGAAGG |
| | | UGAUGGGCCGGCACAAGCCAGAGAACAUCGUGAUCGAGAUGGCCAGGGAGAACCAGACCACUCAAAAGGGGCAGAAGAAC |
| | | UCCAGGGAGCGGAUGAAGCGGAUCGAGGAGGGCAUCAAGGAGCUGGGCUCCCAGAUCCUGAAGGAGCACCCCGUGGAGAA |
| | | CACUCAACUGCAGAACGAGAAGCUGUACCUGUACUACCUGCAGAAUGGGCGAGACAUGUACGUGGACCAGGAGCUGGACA |
| | | UCAACCGGCUCAGCGACUACGACGUGGACCACAUCGUUCCCCAGAGCUUCCUGAAGGACGACAGCAUCGACAACAAGGUG |
| | | CUGACCCGGUCCGACAAGAACCGGGGCAAGUCCGACAACGUUCCCUCAGAGGAAGUCGUGAAGAAGAUGAAGAACUACUG |
| | | GCGGCAGCUGCUGAACGCCAAGCUGAUCACUCAACGGAAGUUCGACAAUCUCACCAAGGCCGAGCGGGGUGGCCUCAGCG |
| | | AGCUGGACAAGGCCGGGUUCAUCAAGCGGCAGCUGGUGGAGACCCGGCAGAUCACCAAGCACGUGGCCCAGAUCCUGGAC |
| | | UCCCGGAUGAACACCAAGUACGACGAGAACGACAAGCUGAUCAGGGAAGUCAAGGUGAUCACCCUGAAGUCCAAGCUGGU |
| | | CAGCGACUUCCGGAAGGACUUCCAGUUCUACAAGGUGAGGGAGAUCAACAACUACCACCACGCCCACGACGCCUACCUGA |
| | | ACGCUGUGGUUGGCACCGCACUGAUCAAGAAGUACCCCAAGCUGGAGUCCGAGUUCGUGUACGGCGACUACAAGGUGUAC |
| | | GACGUGCGGAAGAUGAUAGCCAAGUCCGAGCAGGAGAUCGGCAAGGCCACCGCCAAGUACUUCUUCUACUCCAACAUCAU |
| | | GAACUUCUUCAAGACAGAGAUCACCCUGGCCAAUGGUGAGAUCCGGAAGCGGCCCCUGAUCGAGACCAAUGGUGAGACCG |
| | | GUGAGAUCGUGUGGGACAAGGGGCGAGACUUCGCCACCGUGCGGAAGGUGCUCAGCAUGCCCCAGGUGAACAUCGUGAAG |
| | | AAGACAGAAGUCCAGACCGGUGGCUUCUCCAAGGAGAGCAUCCUUCCAAAGCGGAACUCCGACAAGCUGAUCGCCCGCAA |
| | | GAAGGACUGGGACCCCAAGAAGUACGGUGGCUUCGACUCCCCCACCGUGGCCUACUCCGUGCUGGUGGUGGCCAAGGUGG |
| | | AGAAGGGGAAGUCCAAGAAGCUGAAGUCCGUGAAGGAGCUGCUGGGCAUCACCAUCAUGGAGCGGUCCUCCUUCGAGAAG |
| | | AACCCCAUCGACUUCCUGGAAGCCAAGGGGUACAAGGAAGUCAAGAAGGACCUGAUCAUCAAGCUUCCAAAGUACUCCCU |
| | | GUUCGAGCUGGAGAAUGGGCGGAAGCGGAUGCUGGCCUCCGCCGGUGAGCUGCAGAAGGGGAACGAGCUGGCACUUCCCU |
| | | CAAAGUACGUGAACUUCCUGUACCUGGCCUCCCACUACGAGAAGCUGAAGGGGUCCCCAGAGGACAACGAGCAGAAGCAG |
| | | CUGUUCGUGGAGCAGCACAAGCACUACCUGGACGAGAUCAUCGAGCAGAUCUCCGAGUUCUCCAAGCGGGUGAUCCUGGC |
| | | CGACGCCAAUCUCGACAAGGUGCUCAGCGCCUACAACAAGCACCGAGACAAGCCCAUCAGGGAGCAGGCCGAGAACAUCA |
| | | UCCACCUGUUCACCCUGACCAAUCUCGGUGCCCCCGCUGCCUUCAAGUACUUCGACACCACCAUCGACCGGAAGCGGUACA |
| | | CCUCGACUAAGGAAGUCCUGGACGCCACCCUGAUCCACCAGAGCAUCACCGGCCUGUACGAGACCCGGAUCGACCUCAGCC |
| | | AGCUGGGUGGCGACGGUGGUGGCUCCCCCAAGAAGAAGCGGAAGGUGUAG |
| 47-66 | | Not Used |
| 67 | WT Cas9 ORF | ATGGATAAGAAGTACTCAATCGGGCTGGATATCGGAACTAATTCCGTGGGTTGGGCAGTGATCACGGATGAATACAAAGTGC |
| | | CGTCCAAGAAGTTCAAGGTCCTGGGGAACACCGATAGACACAGCATCAAGAAAAATCTCATCGGAGCCCTGCTGTTTGACTCC |
| | | GGCGAAACCGCAGAAGCGACCCGGCTCAAACGTACCGCGAGGCGACGCTACACCCGGCGGAAGAATCGCATCTGCTATCTGC |
| | | AAGAGATCTTTTCGAACGAAATGGCAAAGGTCGACGACAGCTTCTTCCACCGCCTGGAAGAATCTTTCCTGGTGGAGGAGGAC |
| | | AAGAAGCATGAACGGCATCCTATCTTTGGAAACATCGTCGACGAAGTGGCGTACCACGAAAAGTACCCGACCATCTACCATCT |
| | | GCGGAAGAAGTTGGTTGACTCAACTGACAAGGCCGACCTCAGATTGATCTACTTGGCCCTCGCCCATATGATCAAATTCCGCG |
| | | GACACTTCCTGATCGAAGGCGATCTGAACCCTGATAACTCCGACGTGGATAAGCTTTTCATTCAACTGGTGCAGACCTACAAC |
| | | CAACTGTTCGAAGAAAACCCAATCAATGCTAGCGGCGTCGATGCCAAGGCCATCCTGTCCGCCCGGCTGTCGAAGTCGCGGCG |
| | | CCTCGAAAACCTGATCGCACAGCTGCCGGGAGAGAAAAAGAACGGACTTTTCGGCAACTTGATCGCTCTCTCACTGGGACTCA |
| | | CTCCCAATTTCAAGTCCAATTTTGACCTGGCCGAGGACGCGAAGCTGCAACTCTCAAAGGACACCTACGACGACGACTTGGAC |
| | | AATTTGCTGGCACAAATTGGCGATCAGTACGCGGATCTGTTCCTTGCCGCTAAGAACCTTTCGGACGCAATCTTGCTGTCCGAT |
| | | ATCCTGCGCGTGAACACCGAAATAACCAAAGCGCCGCTTAGCGCCTCGATGATTAAGCGGTACGACGAGCATCACCAGGATC |
| | | TCACGCTGCTCAAAGCGCTCGTGAGACAGCAACTGCCTGAAAAGTACAAGGAGATCTTCTTCGACCAGTCCAAGAATGGGTAC |
| | | GCAGGGTACATCGATGGAGGCGCTAGCCAGGAAGAGTTCTATAAGTTCATCAAGCCAATCCTGGAAAAGATGGACGGAACCG |
| | | AAGAACTGCTGGTCAAGCTGAACAGGGAGGATCTGCTCCGGAAACAGAGAACCTTTGACAACGGATCCATTCCCCACCAGAT |
| | | CCATCTGGGTGAGCTGCACGCCATCTTGCGGCGCCAGGAGGACTTTTACCCATTCCTCAAGGACAACCGGGAAAAGATCGAGA |
| | | AAATTCTGACGTTCCGCATCCCGTATTACGTGGGCCCACTGGCGCGCGGCAATTCGCGCTTCGCGTGGATGACTAGAAAATCA |
| | | GAGGAAACCATCACTCCTTGGAATTTCGAGGAAGTTGTGGATAAGGGAGCTTCGGCACAAAGCTTCATCGAACGAATGACCA |
| | | ACTTCGACAAGAATCTCCCAAACGAGAAGGTGCTTCCTAAGCACAGCCTCCTTTACGAATACTTCACTGTCTACAACGAACTG |
| | | ACTAAAGTGAAATACGTTACTGAAGGAATGAGGAAGCCGGCCTTTCTGTCCGGAGAACAGAAGAAAGCAATTGTCGATCTGC |
| | | TGTTCAAGACCAACCGCAAGGTGACCGTCAAGCAGCTTAAAGAGGACTACTTCAAGAAGATCGAGTGTTTCGACTCAGTGGA |
| | | AATCAGCGGGGTGGAGGACAGATTCAACGCTTCGCTGGGAACCTATCATGATCTCCTGAAGATCATCAAGGACAAGGACTTCC |
| | | TTGACAACGAGGAGAACGAGGACATCCTGGAAGATATCGTCCTGACCTTGACCCTTTTCGAGGATCGCGAGATGATCGAGGA |
| | | GAGGCTTAAGACCTACGCTCATCTCTTCGACGATAAGGTCATGAAACAACTCAAGCGCCGCCGGTACACTGGTTGGGGCCGCC |
| | | TCTCCCGCAAGCTGATCAACGGTATTCGCGATAAACAGAGCGGTAAAACTATCCTGGATTTCCTCAAATCGGATGGCTTCGCT |
| | | AATCGTAACTTCATGCAATTGATCCACGACGACAGCCTGACCTTTAAGGAGGACATCCAAAAAGCACAAGTGTCCGGACAGG |
| | | GAGACTCACTCCATGAACACATCGCGAATCTGGCCGGTTCGCCGGCGATTAAGAAGGGAATTCTGCAAACTGTGAAGGTGGTC |
| | | GACGAGCTGGTGAAGGTCATGGGACGGCACAAACCGGAGAATATCGTGATTGAAATGGCCCGAGAAAACCAGACTACCCAG |
| | | AAGGGCCAGAAAAACTCCCGCGAAAGGATGAAGCGGATCGAAGAAGGAATCAAGGAGCTGGGCAGCCAGATCCTGAAAGAG |
| | | CACCCGGTGGAAAACACGCAGCTGCAGAACGAGAAGCTCTACCTGTACTATTTGCAAAATGGACGGGACATGTACGTGGACC |
| | | AAGAGCTGGACATCAATCGGTTGTCTGATTACGACGTGGACCACATCGTTCCACAGTCCTTTCTGAAGGATGACTCGATCGAT |
| | | AACAAGGTGTTGACTCGCAGCGACAAGAACAGAGGGAAGTCAGATAATGTGCCATCGGAGGAGGTCGTGAAGAAGATGAAG |
| | | AATTACTGGCGGCAGCTCCTGAATGCGAAGCTGATTACCCAGAGAAAGTTTGACAATCTCACTAAAGCCGAGCGCGGCGGAC |
| | | TCTCAGAGCTGGATAAGGCTGGATTCATCAAACGGCAGCTGGTCGAGACTCGGCAGATTACCAAGCACGTGGCGCAGATCTTG |
| | | GACTCCCGCATGAACACTAAATACGACGAGAACGATAAGCTCATCCGGGAAGTGAAGGTGATTACCCTGAAAAGCAAACTTG |
| | | TGTCGGACTTTCGGAAGGACTTTCAGTTTTACAAAGTGAGAGAAATCAACAACTACCATCACGCGCATGACGCATACCTCAAC |
| | | GCTGTGGTCGGTACCGCCCTGATCAAAAAGTACCCTAAACTTGAATCGGAGTTTGTGTACGGAGACTACAAGGTCTACGACGT |
| | | GAGGAAGATGATAGCCAAGTCCGAACAGGAAATCGGGAAAGCAACTGCGAAATACTTCTTTTACTCAAACATCATGAACTTTT |
| | | TCAAGACTGAAATTACGCTGGCCAATGGAGAAATCAGGAAGAGGCCACTGATCGAAACTAACGGAGAAACGGGCGAAATCG |
| | | TGTGGGACAAGGGCAGGGACTTCGCAACTGTTCGCAAAGTGCTCTCTATGCCGCAAGTCAATATTGTGAAGAAAACCGAAGT |
| | | GCAAACCGGCGGATTTTCAAAGGAATCGATCCTCCCAAAGAGAAATAGCGACAAGCTCATTGCACGCAAGAAAGACTGGGAC |
| | | CCGAAGAAGTACGGAGGATTCGATTCGCCGACTGTCGCATACTCCGTCCTCGTGGTGGCCAAGGTGGAGAAGGGAAAGAGCA |
| | | AAAAGCTCAAATCCGTCAAAGAGCTGCTGGGGATTACCATCATGGAACGATCCTCGTTCGAGAAGAACCCGATTGATTTCCTC |
| | | GAGGCGAAGGGTTACAAGGAGGTGAAGAAGGATCTGATCATCAAACTCCCCAAGTACTCACTGTTCGAACTGGAAAATGGTC |
| | | GGAAGCGCATGCTGGCTTCGGCCGGAGAACTCCAAAAAGGAAATGAGCTGGCCTTGCCTAGCAAGTACGTCAACTTCCTCTAT |
| | | CTTGCTTCGCACTACGAAAAACTCAAAGGGTCACCGGAAGATAACGAACAGAAGCAGCTTTTCGTGGAGCAGCACAAGCATT |
| | | ATCTGGATGAAATCATCGAACAAATCTCCGAGTTTTCAAAGCGCGTGATCCTCGCCGACGCCAACCTCGACAAAGTCCTGTCG |
| | | GCCTACAATAAGCATAGAGATAAGCCGATCAGAGAACAGGCCGAGAACATTATCCACTTGTTCACCCTGACTAACCTGGGAG |
| | | CCCCAGCCGCCTTCAAGTACTTCGATACTACTATCGATCGCAAAAGATACACGTCCACCAAGGAAGTTCTGGACGCGACCCTG |
| | | ATCCACCAAAGCATCACTGGACTCTACGAAACTAGGATCGATCTGTCGCAGCTGGGTGGCGAT |
| 68 | WT SERPINA1 | ATGCCGTCTTCTGTCTCGTGGGGCATCCTCCTGCTGGCAGGCCTGTGCTGCCTGGTCCCTGTCTCCCTGGCTGAGGATCCCCAG |
| | ORF | GGAGATGCTGCCCAGAAGACAGATACATCCCACCATGATCAGGATCACCCAACCTTCAACAAGATCACCCCCAACCTGGCTG |
| | | AGTTCGCCTTCAGCCTATACCGCCAGCTGGCACACCAGTCCAACAGCACCAATATCTTCTTCTCCCCAGTGAGCATCGCTACAG |
| | | CCTTTGCAATGCTCTCCCTGGGGACCAAGGCTGACACTCACGATGAAATCCTGGAGGGCCTGAATTTCAACCTCACGGAGATT |
| | | CCGGAGGCTCAGATCCATGAAGGCTTCCAGGAACTCCTCCGTACCCTCAACCAGCCAGACAGCCAGCTCCAGCTGACCACCGG |
| | | CAATGGCCTGTTCCTCAGCGAGGGCCTGAAGCTAGTGGATAAGTTTTTGGAGGATGTTAAAAAGTTGTACCACTCAGAAGCCT |
| | | TCACTGTCAACTTCGGGGACACCGAAGAGGCCAAGAAACAGATCAACGATTACGTGGAGAAGGGTACTCAAGGGAAAATTGT |
| | | GGATTTGGTCAAGGAGCTTGACAGAGACACAGTTTTTGCTCTGGTGAATTACATCTTCTTTAAAGGCAAATGGGAGAGACCCT |
| | | TTGAAGTCAAGGACACCGAGGAAGAGGACTTCCACGTGGACCAGGTGACCACCGTGAAGGTGCCTATGATGAAGCGTTTAGG |
| | | CATGTTTAACATCCAGCACTGTAAGAAGCTGTCCAGCTGGGTGCTGCTGATGAAATACCTGGGCAATGCCACCGCCATCTTCTT |
| | | CCTGCCTGATGAGGGGAAACTACAGCACCTGGAAAATGAACTCACCCACGATATCATCACCAAGTTCCTGGAAAATGAAGAC |
| | | AGAAGGTCTGCCAGCTTACATTTACCCAAACTGTCCATTACTGGAACCTATGATCTGAAGAGCGTCCTGGGTCAACTGGGCAT |
| | | CACTAAGGTCTTCAGCAATGGGGCTGACCTCTCCGGGGTCACAGAGGAGGCACCCCTGAAGCTCTCCAAGGCCGTGCATAAG |
| | | GCTGTGCTGACCATCGACGAGAAAGGGACTGAAGCTGCTGGGGCCATGTTTTTAGAGGCCATACCCATGTCTATCCCCCCCGA |
| | | GGTCAAGTTCAACAAACCCTTTGTCTTCTTAATGATTGAACAAAATACCAAGTCTCCCCTCTTCATGGGAAAAGTGGTGAATCC |
| | | CACCCAAAAATAA |
| 69 | SERPINA1 ORF | ATGCCATCTTCTGTCTCTTGGGGTATCTTGTTGTTGGCCGGTTTGTGCTGCTTGGTCCCAGTCTCTTTGGCCGAAGACCCACAAG |
| | using codons of | GTGACGCCGCCCAAAAGACCGACACCTCTCACCACGACCAAGACCACCCAACCTTCAACAAGATCACCCCAAACTTGGCCGA |
| | Table 5 | ATTCGCCTTCTCTTTGTACAGACAATTGGCCCACCAATCTAACTCTACCAACATCTTCTTCTCTCCAGTCTCTATCGCCACCGCC |
| | | TTCGCCATGTTGTCTTTGGGTACCAAGGCCGACACCCACGACGAAATCTTGGAAGGTTTGAACTTCAACTTGACCGAAATCCC |
| | | AGAAGCCCAAATCCACGAAGGTTTCCAAGAATTGTTGAGAACCTTGAACCAACCAGACTCTCAATTGCAATTGACCACCGGTA |
| | | ACGGTTTGTTCTTGTCTGAAGGTTTGAAGTTGGTCGACAAGTTCTTGGAAGACGTCAAGAAGTTGTACCACTCTGAAGCCTTCA |
| | | CCGTCAACTTCGGTGACACCGAAGAAGCCAAGAAGCAAATCAACGACTACGTCGAAAAGGGTACCCAAGGTAAGATCGTCGA |
| | | CTTGGTCAAGGAATTGGACAGAGACACCGTCTTCGCCTTGGTCAACTACATCTTCTTCAAGGGTAAGTGGGAAAGACCATTCG |
| | | AAGTCAAGGACACCGAAGAAGAAGACTTCCACGTCGACCAAGTCACCACCGTCAAGGTCCCAATGATGAAGAGATTGGGTAT |
| | | GTTCAACATCCAACACTGCAAGAAGTTGTCTTCTTGGGTCTTGTTGATGAAGTACTTGGGTAACGCCACCGCCATCTTCTTCTT |
| | | GCCAGACGAAGGTAAGTTGCAACACTTGGAAAACGAATTGACCCACGACATCATCACCAAGTTCTTGGAAAACGAAGACAGA |
| | | AGATCTGCCTCTTTGCACTTGCCAAAGTTGTCTATCACCGGTACCTACGACTTGAAGTCTGTCTTGGGTCAATTGGGTATCACC |
| | | AAGGTCTTCTCTAACGGTGCCGACTTGTCTGGTGTCACCGAAGAAGCCCCATTGAAGTTGTCTAAGGCCGTCCACAAGGCCGT |
| | | CTTGACCATCGACGAAAAGGGTACCGAAGCCGCCGGTGCCATGTTCTTGGAAGCCATCCCAATGTCTATCCCACCAGAAGTCA |
| | | AGTTCAACAAGCCATTCGTCTTCTTGATGATCGAACAAAACACCAAGTCTCCATTGTTCATGGGTAAGGTCGTCAACCCAACC |
| | | CAAAAGTAA |
| 70 | SERPINA1 ORF | ATGCCGTCGTCGGTCTCGTGGGGAATCCTGCTGCTGGCAGGACTGTGCTGCCTGGTCCCGGTCTCGCTGGCAGAAGACCCGCA |
| | using codons of | GGGAGACGCAGCACAGAAGACAGACACATCGCACCACGACCAGGACCACCCGACATTCAACAAGATCACACCGAACCTGGC |
| | Table 6 | AGAATTCGCATTCTCGCTGTACAGACAGCTGGCACACCAGTCGAACTCGACAAACATCTTCTTCTCGCCGGTCTCGATCGCAA |
| | | CAGCATTCGCAATGCTGTCGCTGGGAACAAAGGCAGACACACACGACGAAATCCTGGAAGGACTGAACTTCAACCTGACAGA |
| | | AATCCCGGAAGCACAGATCCACGAAGGATTCCAGGAACTGCTGAGAACACTGAACCAGCCGGACTCGCAGCTGCAGCTGACA |
| | | ACAGGAAACGGACTGTTCCTGTCGGAAGGACTGAAGCTGGTCGACAAGTTCCTGGAAGACGTCAAGAAGCTGTACCACTCGG |
| | | AAGCATTCACAGTCAACTTCGGAGACACAGAAGAAGCAAAGAAGCAGATCAACGACTACGTCGAAAAGGGAACACAGGGAA |
| | | AGATCGTCGACCTGGTCAAGGAACTGGACAGAGACACAGTCTTCGCACTGGTCAACTACATCTTCTTCAAGGGAAAGTGGGA |
| | | AAGACCGTTCGAAGTCAAGGACACAGAAGAAGAAGACTTCCACGTCGACCAGGTCACAACAGTCAAGGTCCCGATGATGAAG |
| | | AGACTGGGAATGTTCAACATCCAGCACTGCAAGAAGCTGTCGTCGTGGGTCCTGCTGATGAAGTACCTGGGAAACGCAACAG |
| | | CAATCTTCTTCCTGCCGGACGAAGGAAAGCTGCAGCACCTGGAAAACGAACTGACACACGACATCATCACAAAGTTCCTGGA |
| | | AAACGAAGACAGAAGATCGGCATCGCTGCACCTGCCGAAGCTGTCGATCACAGGAACATACGACCTGAAGTCGGTCCTGGGA |
| | | CAGCTGGGAATCACAAAGGTCTTCTCGAACGGAGCAGACCTGTCGGGAGTCACAGAAGAAGCACCGCTGAAGCTGTCGAAGG |
| | | CAGTCCACAAGGCAGTCCTGACAATCGACGAAAAGGGAACAGAAGCAGCAGGAGCAATGTTCCTGGAAGCAATCCCGATGTC |
| | | GATCCCGCCGGAAGTCAAGTTCAACAAGCCGTTCGTCTTCCTGATGATCGAACAGAACACAAAGTCGCCGCTGTTCATGGGAA |
| | | AGGTCGTCAACCCGACACAGAAGTGA |
| 71 | SERPINA1 ORF | ATGCCCAGCAGCGTGAGCTGGGGCATCCTGCTGCTGGCCGGCCTGTGCTGCCTGGTGCCCGTGAGCCTGGCCGAGGACCCCCA |
| | 1.1 | GGGCGACGCCGCCCAGAAGACGGACACGAGCCACCACGACCAGGACCACCCCACGTTCAACAAGATCACGCCCAACCTGGCC |
| | | GAGTTCGCCTTCAGCCTGTACCGGCAGCTGGCCCACCAGAGCAACAGCACGAACATCTTCTTCAGCCCCGTGAGCATCGCCAC |
| | | GGCCTTCGCCATGCTGAGCCTGGGCACGAAGGCCGACACGCACGACGAGATCCTGGAGGGCCTGAACTTCAACCTGACGGAG |
| | | ATCCCCGAGGCCCAGATCCACGAGGGCTTCCAGGAGCTGCTGCGGACGCTGAACCAGCCCGACAGCCAGCTGCAGCTGACGA |
| | | CGGGCAACGGCCTGTTCCTGAGCGAGGGCCTGAAGCTGGTGGACAAGTTCCTGGAGGACGTGAAGAAGCTGTACCACAGCGA |
| | | GGCCTTCACGGTGAACTTCGGCGACACGGAGGAGGCCAAGAAGCAGATCAACGACTACGTGGAGAAGGGCACGCAGGGCAA |
| | | GATCGTGGACCTGGTGAAGGAGCTGGACCGGGACACGGTGTTCGCCCTGGTGAACTACATCTTCTTCAAGGGCAAGTGGGAG |
| | | CGGCCCTTCGAGGTGAAGGACACGGAGGAGGAGGACTTCCACGTGGACCAGGTGACGACGGTGAAGGTGCCCATGATGAAGC |
| | | GGCTGGGCATGTTCAACATCCAGCACTGCAAGAAGCTGAGCAGCTGGGTGCTGCTGATGAAGTACCTGGGCAACGCCACGGC |
| | | CATCTTCTTCCTGCCCGACGAGGGCAAGCTGCAGCACCTGGAGAACGAGCTGACGCACGACATCATCACGAAGTTCCTGGAGA |
| | | ACGAGGACCGGCGGAGCGCCAGCCTGCACCTGCCCAAGCTGAGCATCACGGGCACGTACGACCTGAAGAGCGTGCTGGGCCA |
| | | GCTGGGCATCACGAAGGTGTTCAGCAACGGCGCCGACCTGAGCGGCGTGACGGAGGAGGCCCCCCTGAAGCTGAGCAAGGCC |
| | | GTGCACAAGGCCGTGCTGACGATCGACGAGAAGGGCACGGAGGCCGCCGGCGCCATGTTCCTGGAGGCCATCCCCATGAGCA |
| | | TCCCCCCCGAGGTGAAGTTCAACAAGCCCTTCGTGTTCCTGATGATCGAGCAGAACACGAAGAGCCCCCTGTTCATGGGCAAG |
| | | GTGGTGAACCCCACGCAGAAGTAG |
| 72 | SERPINA1 ORF | ATGCCCTCGTCGGTCTCGTGGGGCATCCTCCTCCTCGCGGGCCTCTGCTGCCTCGTCCCCGTCTCGCTCGCGGAGGACCCCCAG |
| | 1.2 | GGCGACGCGGCGCAGAAGACGGACACGTCGCACCACGACCAGGACCACCCCACGTTCAACAAGATCACGCCCAACCTCGCGG |
| | | AGTTCGCGTTCTCGCTCTACCGCCAGCTCGCGCACCAGTCGAACTCGACGAACATCTTCTTCTCGCCCGTCTCGATCGCGACGG |
| | | CGTTCGCGATGCTCTCGCTCGGCACGAAGGCGGACACGCACGACGAGATCCTCGAGGGCCTCAACTTCAACCTCACGGAGATC |
| | | CCCGAGGCGCAGATCCACGAGGGCTTCCAGGAGCTCCTCCGCACGCTCAACCAGCCCGACTCGCAGCTCCAGCTCACGACGG |
| | | GCAACGGCCTCTTCCTCTCGGAGGGCCTCAAGCTCGTCGACAAGTTCCTCGAGGACGTCAAGAAGCTCTACCACTCGGAGGCG |
| | | TTCACGGTCAACTTCGGCGACACGGAGGAGGCGAAGAAGCAGATCAACGACTACGTCGAGAAGGGCACGCAGGGCAAGATC |
| | | GTCGACCTCGTCAAGGAGCTCGACCGCGACACGGTCTTCGCGCTCGTCAACTACATCTTCTTCAAGGGCAAGTGGGAGCGCCC |
| | | CTTCGAGGTCAAGGACACGGAGGAGGAGGACTTCCACGTCGACCAGGTCACGACGGTCAAGGTCCCCATGATGAAGCGCCTC |
| | | GGCATGTTCAACATCCAGCACTGCAAGAAGCTCTCGTCGTGGGTCCTCCTCATGAAGTACCTCGGCAACGCGACGGCGATCTT |
| | | CTTCCTCCCCGACGAGGGCAAGCTCCAGCACCTCGAGAACGAGCTCACGCACGACATCATCACGAAGTTCCTCGAGAACGAG |
| | | GACCGCCGCTCGGCGTCGCTCCACCTCCCCAAGCTCTCGATCACGGGCACGTACGACCTCAAGTCGGTCCTCGGCCAGCTCGG |
| | | CATCACGAAGGTCTTCTCGAACGGCGCGGACCTCTCGGGCGTCACGGAGGAGGCGCCCCTCAAGCTCTCGAAGGCGGTCCAC |
| | | AAGGCGGTCCTCACGATCGACGAGAAGGGCACGGAGGCGGCGGGCGCGATGTTCCTCGAGGCGATCCCCATGTCGATCCCCC |
| | | CCGAGGTCAAGTTCAACAAGCCCTTCGTCTTCCTCATGATCGAGCAGAACACGAAGTCGCCCCTCTTCATGGGCAAGGTCGTC |
| | | AACCCCACGCAGAAGTAG |
| 73 | SERPINA1 ORF | ATGCCCTCATCGGTCAGCTGGGGCATCCTCCTCCTCGCCGGGCTCTGCTGCCTCGTTCCCGTCAGCCTCGCGGAGGACCCCCAG |
| | 1.3 | GGCGACGCTGCCCAGAAGACGGACACGTCGCACCACGACCAGGACCACCCCACCTTCAACAAGATCACTCCCAATCTCGCGG |
| | | AGTTCGCGTTCTCGCTCTACCGCCAGCTCGCGCACCAGAGCAACTCGACTAACATCTTCTTCTCGCCCGTCAGCATCGCGACGG |
| | | CGTTCGCGATGCTCAGCCTCGGCACGAAGGCGGACACGCACGACGAGATCCTCGAGGGCCTCAACTTCAATCTCACAGAGATC |
| | | CCAGAAGCCCAGATCCACGAGGGCTTCCAGGAGCTGCTGCGGACGCTCAACCAGCCTGACTCGCAGCTCCAGCTCACGACGG |
| | | GCAATGGGCTCTTCCTCAGCGAGGGCCTCAAGCTCGTCGACAAGTTCCTGGAGGACGTCAAGAAGCTCTACCACTCGGAAGCC |
| | | TTCACGGTCAACTTCGGCGACACAGAGGAAGCCAAGAAGCAGATCAACGACTACGTCGAGAAGGGGACTCAGGGCAAGATC |
| | | GTCGACCTCGTCAAGGAGCTGGACCGAGACACGGTCTTCGCACTGGTCAACTACATCTTCTTCAAGGGGAAGTGGGAGCGCCC |
| | | CTTCGAAGTCAAGGACACAGAGGAGGAGGACTTCCACGTCGACCAGGTGACGACGGTCAAGGTTCCCATGATGAAGCGCCTC |
| | | GGCATGTTCAACATCCAGCACTGCAAGAAGCTCAGCTCGTGGGTCCTCCTCATGAAGTACCTCGGCAACGCGACGGCGATCTT |
| | | CTTCCTTCCTGACGAGGGCAAGCTCCAGCACCTCGAGAACGAGCTGACGCACGACATCATCACGAAGTTCCTGGAGAACGAG |
| | | GACCGCCGATCGGCGTCGCTCCACCTTCCAAAGCTCAGCATCACGGGCACCTACGACCTCAAGTCGGTCCTCGGCCAGCTCGG |
| | | CATCACGAAGGTCTTCTCGAATGGTGCCGACCTCAGCGGCGTCACAGAGGAAGCCCCCCTCAAGCTCAGCAAGGCTGTGCACA |
| | | AGGCTGTGCTCACGATCGACGAGAAGGGGACAGAAGCTGCCGGTGCCATGTTCCTGGAAGCCATCCCCATGAGCATCCCACC |
| | | AGAAGTCAAGTTCAACAAGCCCTTCGTCTTCCTGATGATAGAGCAGAACACGAAGTCGCCCCTCTTCATGGGCAAGGTCGTCA |
| | | ACCCCACTCAAAAGTAG |
| 74 | SERPINA1 WT | MPSSVSWGILLLAGLCCLVPVSLAEDPQGDAAQKTDTSHHDQDHPTFNKITPNLAEFAFSLYRQLAHQSNSTNIFFSPVSIATAFAML |
| | amino acid | SLGTKADTHDEILEGLNFNLTEIPEAQIHEGFQELLRTLNQPDSQLQLTTGNGLFLSEGLKLVDKFLEDVKKLYHSEAFTVNFGDTEE |
| | sequence | AKKQINDYVEKGTQGKIVDLVKELDRDTVFALVNYIFFKGKWERPFE,VKDTEEEDFHVDQVTTVKVPMMKRLGMFNIQHCKKLSS |
| | | WVLLMKYLGNATAIFFLPDEGKLQHLENELTHDIITKFLENEDRRSASLHLPKLSITGTYDLKSVLGQLGITKVFSNGADLSGVTEEA |
| | | PLKLSKAVHKAVLTIDEKGTEAAGAMFLEAIPMSIPPEVKFNKPFVFLMIEQNTKSPLFMGKVVNPTQK |
| 75 | | Not Used |
| 76 | SERPINA1 | GGGTCCCGCAGTCGGCGTCCAGCGGCTCTGCTTGTTCGTGTGTGTGTCGTTGCAGGCCTTATTCGGATCaGCCACCATGCCGTC |
| | transcript | GTCGGTCTCGTGGGGAATCCTGCTGCTGGCAGGACTGTGCTGCCTGGTCCCGGTCTCGCTGGCAGAAGACCCGCAGGGAGACG |
| | comprising SEQ | CAGCACAGAAGACAGACACATCGCACCACGACCAGGACCACCCGACATTCAACAAGATCACACCGAACCTGGCAGAATTCGC |
| | 70 | ATTCTCGCTGTACAGACAGCTGGCACACCAGTCGAACTCGACAAACATCTTCTTCTCGCCGGTCTCGATCGCAACAGCATTCGC |
| | | AATGCTGTCGCTGGGAACAAAGGCAGACACACACGACGAAATCCTGGAAGGACTGAACTTCAACCTGACAGAAATCCCGGAA |
| | | GCACAGATCCACGAAGGATTCCAGGAACTGCTGAGAACACTGAACCAGCCGGACTCGCAGCTGCAGCTGACAACAGGAAACG |
| | | GACTGTTCCTGTCGGAAGGACTGAAGCTGGTCGACAAGTTCCTGGAAGACGTCAAGAAGCTGTACCACTCGGAAGCATTCACA |
| | | GTCAACTTCGGAGACACAGAAGAAGCAAAGAAGCAGATCAACGACTACGTCGAAAAGGGAACACAGGGAAAGATCGTCGAC |
| | | CTGGTCAAGGAACTGGACAGAGACACAGTCTTCGCACTGGTCAACTACATCTTCTTCAAGGGAAAGTGGGAAAGACCGTTCG |
| | | AAGTCAAGGACACAGAAGAAGAAGACTTCCACGTCGACCAGGTCACAACAGTCAAGGTCCCGATGATGAAGAGACTGGGAA |
| | | TGTTCAACATCCAGCACTGCAAGAAGCTGTCGTCGTGGGTCCTGCTGATGAAGTACCTGGGAAACGCAACAGCAATCTTCTTC |
| | | CTGCCGGACGAAGGAAAGCTGCAGCACCTGGAAAACGAACTGACACACGACATCATCACAAAGTTCCTGGAAAACGAAGAC |
| | | AGAAGATCGGCATCGCTGCACCTGCCGAAGCTGTCGATCACAGGAACATACGACCTGAAGTCGGTCCTGGGACAGCTGGGAA |
| | | TCACAAAGGTCTTCTCGAACGGAGCAGACCTGTCGGGAGTCACAGAAGAAGCACCGCTGAAGCTGTCGAAGGCAGTCCACAA |
| | | GGCAGTCCTGACAATCGACGAAAAGGGAACAGAAGCAGCAGGAGCAATGTTCCTGGAAGCAATCCCGATGTCGATCCCGCCG |
| | | GAAGTCAAGTTCAACAAGCCGTTCGTCTTCCTGATGATCGAACAGAACACAAAGTCGCCGCTGTTCATGGGAAAGGTCGTCAA |
| | | CCCGACACAGAAGTagCTAGCCATCACATTTAAAAGCATCTCAGCCTACCATGAGAATAAGAGAAAGAAAATGAAGATCAATA |
| | | GCTTATTCATCTCITTTTCTTTTTCGTTGGTGTAAAGCCAACACCCTGTCTAAAAAACATAAATTTCTTTAATCATTTTGCCTCTT |
| | | TTCTCTGTGCTTCAATTAATAAAAAATGGAAAGAACCTCGAGAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAATCTAG |
| 77 | SERPINA1 | GGGTCCCGCAGTCGGCGTCCAGCGGCTCTGCTTGTTCGTGTGTGTGTCGTTGCAGGCCTTATTCGGATCaGCCACCATGCCATC |
| | transcript | TTCTGTCTCTTGGGGTATCTTGTTGTTGGCCGGTTTGTGCTGCTTGGTCCCAGTCTCITTGGCCGAAGACCCACAAGGTGACGCC |
| | comprising SEQ | GCCCAAAAGACCGACACCTCTCACCACGACCAAGACCACCCAACCTTCAACAAGATCACCCCAAACTTGGCCGAATTCGCCTT |
| | 69 | CTCTTTGTACAGACAATTGGCCCACCAATCTAACTCTACCAACATCTTCTTCTCTCCAGTCTCTATCGCCACCGCCTTCGCCATG |
| | | TTGTCTTTGGGTACCAAGGCCGACACCCACGACGAAATCTTGGAAGGTTTGAACTTCAACTTGACCGAAATCCCAGAAGCCCA |
| | | AATCCACGAAGGITTCCAAGAATTGTTGAGAACCTTGAACCAACCAGACTCTCAATTGCAATTGACCACCGGTAACGGTTTGT |
| | | TCTTGTCTGAAGGTTTGAAGTTGGTCGACAAGTTCTTGGAAGACGTCAAGAAGTTGTACCACTCTGAAGCCTTCACCGTCAACT |
| | | TCGGTGACACCGAAGAAGCCAAGAAGCAAATCAACGACTACGTCGAAAAGGGTACCCAAGGTAAGATCGTCGACTTGGTCAA |
| | | GGAATTGGACAGAGACACCGTCTTCGCCTTGGTCAACTACATCTTCTTCAAGGGTAAGTGGGAAAGACCATTCGAAGTCAAGG |
| | | ACACCGAAGAAGAAGACTTCCACGTCGACCAAGTCACCACCGTCAAGGTCCCAATGATGAAGAGATTGGGTATGITCAACAT |
| | | CCAACACTGCAAGAAGTTGTCTTCTTGGGTCTTGTTGATGAAGTACTTGGGTAACGCCACCGCCATCTTCTTCTTGCCAGACGA |
| | | AGGTAAGTTGCAACACTTGGAAAACGAATTGACCCACGACATCATCACCAAGTTCTTGGAAAACGAAGACAGAAGATCTGCC |
| | | TCTTTGCACTTGCCAAAGTTGTCTATCACCGGTACCTACGACTTGAAGTCTGTCTTGGGTCAATTGGGTATCACCAAGGTCTTC |
| | | TCTAACGGTGCCGACTTGTCTGGTGTCACCGAAGAAGCCCCATTGAAGTTGTCTAAGGCCGTCCACAAGGCCGTCTTGACCAT |
| | | CGACGAAAAGGGTACCGAAGCCGCCGGTGCCATGTTCTTGGAAGCCATCCCAATGTCTATCCCACCAGAAGTCAAGTTCAACA |
| | | AGCCATTCGTCTTCTTGATGATCGAACAAAACACCAAGTCTCCATTGTTCATGGGTAAGGTCGTCAACCCAACCCAAAAGTAgC |
| | | TAGCCATCACATTTAAAAGCATCTCAGCCTACCATGAGAATAAGAGAAAGAAAATGAAGATCAATAGCTTATTCATCTCTTTT |
| | | TCTTTTTCGTTGGTGTAAAGCCAACACCCTGTCTAAAAAACATAAATTTCTTTAATCATTTTGCCTCTTTTCTCTGTGCTTCAATT |
| | | AATAAAAAATGGAAAGAACCTCGAGAAAAAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAATCTAG |
| 78 | SERPINA1 | GGGTCCCGCAGTCGGCGTCCAGCGGCTCTGCTTGTTCGTGTGTGTGTCGTTGCAGGCCTTATTCCGCCACCATGCCCAGCAGCG |
| | transcript | TGAGCTGGGGCATCCTGCTGCTGGCCGGCCTGTGCTGCCTGGTGCCCGTGAGCCTGGCCGAGGACCCCCAGGGCGACGCCGCC |
| | comprising SEQ | CAGAAGACGGACACGAGCCACCACGACCAGGACCACCCCACGTTCAACAAGATCACGCCCAACCTGGCCGAGTTCGCCTTCA |
| | 71 | GCCTGTACCGGCAGCTGGCCCACCAGAGCAACAGCACGAACATCTTCTTCAGCCCCGTGAGCATCGCCACGGCCTTCGCCATG |
| | | CTGAGCCTGGGCACGAAGGCCGACACGCACGACGAGATCCTGGAGGGCCTGAACTTCAACCTGACGGAGATCCCCGAGGCCC |
| | | AGATCCACGAGGGCTTCCAGGAGCTGCTGCGGACGCTGAACCAGCCCGACAGCCAGCTGCAGCTGACGACGGGCAACGGCCT |
| | | GTTCCTGAGCGAGGGCCTGAAGCTGGTGGACAAGTTCCTGGAGGACGTGAAGAAGCTGTACCACAGCGAGGCCTTCACGGTG |
| | | AACTTCGGCGACACGGAGGAGGCCAAGAAGCAGATCAACGACTACGTGGAGAAGGGCACGCAGGGCAAGATCGTGGACCTG |
| | | GTGAAGGAGCTGGACCGGGACACGGTGTTCGCCCTGGTGAACTACATCTTCTTCAAGGGCAAGTGGGAGCGGCCCTTCGAGGT |
| | | GAAGGACACGGAGGAGGAGGACTTCCACGTGGACCAGGTGACGACGGTGAAGGTGCCCATGATGAAGCGGCTGGGCATGTTC |
| | | AACATCCAGCACTGCAAGAAGCTGAGCAGCTGGGTGCTGCTGATGAAGTACCTGGGCAACGCCACGGCCATCTTCTTCCTGCC |
| | | CGACGAGGGCAAGCTGCAGCACCTGGAGAACGAGCTGACGCACGACATCATCACGAAGTTCCTGGAGAACGAGGACCGGCG |
| | | GAGCGCCAGCCTGCACCTGCCCAAGCTGAGCATCACGGGCACGTACGACCTGAAGAGCGTGCTGGGCCAGCTGGGCATCACG |
| | | AAGGTGTTCAGCAACGGCGCCGACCTGAGCGGCGTGACGGAGGAGGCCCCCCTGAAGCTGAGCAAGGCCGTGCACAAGGCCG |
| | | TGCTGACGATCGACGAGAAGGGCACGGAGGCCGCCGGCGCCATGTTCCTGGAGGCCATCCCCATGAGCATCCCCCCCGAGGT |
| | | GAAGTTCAACAAGCCCTTCGTGTTCCTGATGATCGAGCAGAACACGAAGAGCCCCCTGTTCATGGGCAAGGTGGTGAACCCCA |
| | | CGCAGAAGTAGTAGTGAAGCTTCTAGCCATCACATTTAAAAGCATCTCAGCCTACCATGAGAATAAGAGAAAGAAAATGAAG |
| | | ATCAATAGCTTATTCATCTCTTTTTCTTTTTCGTTGGTGTAAAGCCAACACCCTGTCTAAAAAACATAAATTTCTTTAATCATTT |
| | | TGCCTCTTTTCTCTGTGCTTCAATTAATAAAAAATGGAAAGAACCTCGAGAAAAAAAAAAAAAAAAAAAAAAAAAAAAAAGC |
| | | GAAAAAAAAAAAAAAAAAAAAAAAAAAAAAACCGAAAAAAAAAAAAAAAAAAAAAAAAAAAAAAAAAAAAAAAT |
| 79 | SERPINA1 | GGGTCCCGCAGTCGGCGTCCAGCGGCTCTGCTTGTTCGTGTGTGTGTCGTTGCAGGCCTTATTCCGCCACCATGCCCTCGTCGG |
| | transcript | TCTCGTGGGGCATCCTCCTCCTCGCGGGCCTCTGCTGCCTCGTCCCCGTCTCGCTCGCGGAGGACCCCCAGGGCGACGCGGCGC |
| | comprising SEQ | AGAAGACGGACACGTCGCACCACGACCAGGACCACCCCACGTTCAACAAGATCACGCCCAACCTCGCGGAGTTCGCGTTCTC |
| | 72 | GCTCTACCGCCAGCTCGCGCACCAGTCGAACTCGACGAACATCTTCTTCTCGCCCGTCTCGATCGCGACGGCGTTCGCGATGCT |
| | | CTCGCTCGGCACGAAGGCGGACACGCACGACGAGATCCTCGAGGGCCTCAACTTCAACCTCACGGAGATCCCCGAGGCGCAG |
| | | ATCCACGAGGGCTTCCAGGAGCTCCTCCGCACGCTCAACCAGCCCGACTCGCAGCTCCAGCTCACGACGGGCAACGGCCTCTT |
| | | CCTCTCGGAGGGCCTCAAGCTCGTCGACAAGTTCCTCGAGGACGTCAAGAAGCTCTACCACTCGGAGGCGTTCACGGTCAACT |
| | | TCGGCGACACGGAGGAGGCGAAGAAGCAGATCAACGACTACGTCGAGAAGGGCACGCAGGGCAAGATCGTCGACCTCGTCA |
| | | AGGAGCTCGACCGCGACACGGTCTTCGCGCTCGTCAACTACATCTTCTTCAAGGGCAAGTGGGAGCGCCCCTTCGAGGTCAAG |
| | | GACACGGAGGAGGAGGACTTCCACGTCGACCAGGTCACGACGGTCAAGGTCCCCATGATGAAGCGCCTCGGCATGTTCAACA |
| | | TCCAGCACTGCAAGAAGCTCTCGTCGTGGGTCCTCCTCATGAAGTACCTCGGCAACGCGACGGCGATCTTCTTCCTCCCCGACG |
| | | AGGGCAAGCTCCAGCACCTCGAGAACGAGCTCACGCACGACATCATCACGAAGTTCCTCGAGAACGAGGACCGCCGCTCGGC |
| | | GTCGCTCCACCTCCCCAAGCTCTCGATCACGGGCACGTACGACCTCAAGTCGGTCCTCGGCCAGCTCGGCATCACGAAGGTCT |
| | | TCTCGAACGGCGCGGACCTCTCGGGCGTCACGGAGGAGGCGCCCCTCAAGCTCTCGAAGGCGGTCCACAAGGCGGTCCTCAC |
| | | GATCGACGAGAAGGGCACGGAGGCGGCGGGCGCGATGTTCCTCGAGGCGATCCCCATGTCGATCCCCCCCGAGGTCAAGTTC |
| | | AACAAGCCCTTCGTCTTCCTCATGATCGAGCAGAACACGAAGTCGCCCCTCTTCATGGGCAAGGTCGTCAACCCCACGCAGAA |
| | | GTAGTAGTAGAGCTTCTAGCCATCACATTTAAAAGCATCTCAGCCTACCATGAGAATAAGAGAAAGAAAATGAAGATCAATA |
| | | GCTTATTCATCTCTTTTTCTTTTTCGTTGGTGTAAAGCCAACACCCTGTCTAAAAAACATAAATTTCTTTAATCATTTTGCCTCTT |
| | | TTCTCTGTGCTTCAATTAATAAAAAATGGAAAGAACCTCGAGAAAAAAAAAAAAAAAAAAAAAAAAAAAAAAGCGAAAAAA |
| | | AAAAAAAAAAAAAAAAAAAAAAAACCGAAAAAAAAAAAAAAAAAAAAAAAAAAAAAAAAAAAAAAAT |
| 80 | SERPINA1 | GGGTCCCGCAGTCGGCGTCCAGCGGCTCTGCTTGTTCGTGTGTGTGTCGTTGCAGGCCTTATTCCGCCACCATGCCCTCATCGG |
| | transcript | TCAGCTGGGGCATCCTCCTCCTCGCCGGGCTCTGCTGCCTCGTTCCCGTCAGCCTCGCGGAGGACCCCCAGGGCGACGCTGCCC |
| | comprising SEQ | AGAAGACGGACACGTCGCACCACGACCAGGACCACCCCACCTTCAACAAGATCACTCCCAATCTCGCGGAGTTCGCGTTCTCG |
| | 73 | CTCTACCGCCAGCTCGCGCACCAGAGCAACTCGACTAACATCTTCTTCTCGCCCGTCAGCATCGCGACGGCGTTCGCGATGCTC |
| | | AGCCTCGGCACGAAGGCGGACACGCACGACGAGATCCTCGAGGGCCTCAACTTCAATCTCACAGAGATCCCAGAAGCCCAGA |
| | | TCCACGAGGGCTTCCAGGAGCTGCTGCGGACGCTCAACCAGCCTGACTCGCAGCTCCAGCTCACGACGGGCAATGGGCTCTTC |
| | | CTCAGCGAGGGCCTCAAGCTCGTCGACAAGTTCCTGGAGGACGTCAAGAAGCTCTACCACTCGGAAGCCTTCACGGTCAACTT |
| | | CGGCGACACAGAGGAAGCCAAGAAGCAGATCAACGACTACGTCGAGAAGGGGACTCAGGGCAAGATCGTCGACCTCGTCAA |
| | | GGAGCTGGACCGAGACACGGTCTTCGCACTGGTCAACTACATCTTCTTCAAGGGGAAGTGGGAGCGCCCCTTCGAAGTCAAGG |
| | | ACACAGAGGAGGAGGACTTCCACGTCGACCAGGTGACGACGGTCAAGGTTCCCATGATGAAGCGCCTCGGCATGTTCAACAT |
| | | CCAGCACTGCAAGAAGCTCAGCTCGTGGGTCCTCCTCATGAAGTACCTCGGCAACGCGACGGCGATCTTCTTCCTTCCTGACG |
| | | AGGGCAAGCTCCAGCACCTCGAGAACGAGCTGACGCACGACATCATCACGAAGTTCCTGGAGAACGAGGACCGCCGATCGGC |
| | | GTCGCTCCACCTTCCAAAGCTCAGCATCACGGGCACCTACGACCTCAAGTCGGTCCTCGGCCAGCTCGGCATCACGAAGGTCT |
| | | TCTCGAATGGTGCCGACCTCAGCGGCGTCACAGAGGAAGCCCCCCTCAAGCTCAGCAAGGCTGTGCACAAGGCTGTGCTCACG |
| | | ATCGACGAGAAGGGGACAGAAGCTGCCGGTGCCATGTTCCTGGAAGCCATCCCCATGAGCATCCCACCAGAAGTCAAGTTCA |
| | | ACAAGCCCTTCGTCTTCCTGATGATAGAGCAGAACACGAAGTCGCCCCTCTTCATGGGCAAGGTCGTCAACCCCACTCAAAAG |
| | | TAGTGATAGAGCTTCTAGCCATCACATTTAAAAGCATCTCAGCCTACCATGAGAATAAGAGAAAGAAAATGAAGATCAATAG |
| | | CTTATTCATCTCTTTTTCTTTTTCGTTGGTGTAAAGCCAACACCCTGTCTAAAAAACATAAATTTCTTTAATCATTTTGCCTCTTT |
| | | TCTCTGTGCTTCAATTAATAAAAAATGGAAAGAACCTCGAGAAAAAAAAAAAAAAAAAAAAAAAAAAAAAAGCGAAAAAAA |
| | | AAAAAAAAAAAAAAAAAAAAAAACCGAAAAAAAAAAAAAAAAAAAAAAAAAAAAAAAAAAAAAAAT |
| 81-87 | | Not Used |
| 88 | FAH amino acid | MSFIPVAEDSDFPIHNLPYGVFSTRGDPRPRIGVAIGDQILDLSIIKHLFTGPVLSKHQDVFNQPTLNSFMGLGQAAWKEARVFLQNLL |
| | sequence | SVSQARLRDDTELRKCAFISQASATMHLPATIGDYTDFYSSRQHATNVGIMFRDKENALMPNWLHLPVGYHGRASSVVVSGTPIRR |
| | | PMGQMKPDDSKPPVYGACKLLDMELEMAFFVGPGNRLGEPIPISKAHEHIFGMVLMNDWSARDIQKWEYVPLGPFLGKSFGTTVSP |
| | | WVVPMDALMPFAVPNPKQDPRPLPYLCHDEPYTFDINLSVNLKGEGMSQAATICKSNFKYMYWTMLQQLTHHSVNGCNLRPGDL |
| | | LASGTISGPEPENFGSMLELSWKGTKPIDLGNGQTRKFLLDGDEVIITGYCQGDGYRIGFGQCAGKVLPALLP |
| 89 | FAH WT ORF | AUGUCUUUUAUUCCUGUUGCUGAAGAUUCUGAUUUUCCUAUUCAUAAUUUACCUUAUGGUGUUUUUUCUACUCGUGGUG |
| | | AUCCUCGUCCUCGUAUUGGUGUUGCUAUUGGUGAUCAAAUUUUAGAUUUAUCUAUUAUUAAACAUUUAUUUACUGGUCC |
| | | UGUUUUAUCUAAACAUCAAGAUGUUUUUAAUCAACCUACUUUAAAUUCUUUUAUGGGUUUAGGUCAAGCUGCUUGGAAA |
| | | GAAGCUCGUGUUUUUUUACAAAAUUUAUUAUCUGUUUCUCAAGCUCGUUUACGUGAUGAUACUGAAUUACGUAAAUGUG |
| | | CUUUUAUUUCUCAAGCUUCUGCUACUAUGCAUUUACCUGCUACUAUUGGUGAUUAUACUGAUUUUUAUUCUUCUCGUCAA |
| | | CAUGCUACUAAUGUUGGUAUUAUGUUUCGUGAUAAAGAAAAUGCUUUAAUGCCUAAUUGGUUACAUUUACCUGUUGGUU |
| | | AUCAUGGUCGUGCUUCUUCUGUUGUUGUUUCUGGUACUCCUAUUCGUCGUCCUAUGGGUCAAAUGAAACCUGAUGAUUCU |
| | | AAACCUCCUGUUUAUGGUGCUUGUAAAUUAUUAGAUAUGGAAUUAGAAAUGGCUUUUUUUGUUGGUCCUGGUAAUCGUU |
| | | UAGGUGAACCUAUUCCUAUUUCUAAAGCUCAUGAACAUAUUUUUGGUAUGGUUUUAAUGAAUGAUUGGUCUGCUCGUGA |
| | | UAUUCAAAAAUGGGAAUAUGUUCCUUUAGGUCCUUUUUUAGGUAAAUCUUUUGGUACUACUGUUUCUCCUUGGGUUGUU |
| | | CCUAUGGAUGCUUUAAUGCCUUUUGCUGUUCCUAAUCCUAAACAAGAUCCUCGUCCUUUACCUUAUUUAUGUCAUGAUGA |
| | | ACCUUAUACUUUUGAUAUUAAUUUAUCUGUUAAUUUAAAAGGUGAAGGUAUGUCUCAAGCUGCUACUAUUUGUAAAUCU |
| | | AAUUUUAAAUAUAUGUAUUGGACUAUGUUACAACAAUUAACUCAUCAUUCUGUUAAUGGUUGUAAUUUACGUCCUGGUG |
| | | AUUUAUUAGCUUCUGGUACUAUUUCUGGUCCUGAACCUGAAAAUUUUGGUUCUAUGUUAGAAUUAUCUUGGAAAGGUAC |
| | | UAAACCUAUUGAUUUAGGUAAUGGUCAAACUCGUAAAUUUUUAUUAGAUGGUGAUGAAGUUAUUAUUACUGGUUAUUGU |
| | | CAAGGUGAUGGUUAUCGUAUUGGUUUUGGUCAAUGUGCUGGUAAAGUUUUACCUGCUUUAUUACCUUAG |
| 90 | FAH BP_GCU | ATGTCGTTCATCCCCGTCGCGGAGGACTCGGACTTCCCCATCCACAACCTCCCCTACGGCGTCTTCTCGACGCGCGGCGACCCC |
| | ORF | CGCCCCCGCATCGGCGTCGCGATCGGCGACCAGATCCTCGACCTCTCGATCATCAAGCACCTCTTCACGGGCCCCGTCCTCTCG |
| | | AAGCACCAGGACGTCTTCAACCAGCCCACGCTCAACTCGTTCATGGGCCTCGGCCAGGCGGCGTGGAAGGAGGCGCGCGTCTT |
| | | CCTCCAGAACCTCCTCTCGGTCTCGCAGGCGCGCCTCCGCGACGACACGGAGCTCCGCAAGTGCGCGTTCATCTCGCAGGCGT |
| | | CGGCGACGATGCACCTCCCCGCGACGATCGGCGACTACACGGACTTCTACTCGTCGCGCCAGCACGCGACGAACGTCGGCATC |
| | | ATGTTCCGCGACAAGGAGAACGCGCTCATGCCCAACTGGCTCCACCTCCCCGTCGGCTACCACGGCCGCGCGTCGTCGGTCGT |
| | | CGTCTCGGGCACGCCCATCCGCCGCCCCATGGGCCAGATGAAGCCCGACGACTCGAAGCCCCCCGTCTACGGCGCGTGCAAGC |
| | | TCCTCGACATGGAGCTCGAGATGGCGTTCTTCGTCGGCCCCGGCAACCGCCTCGGCGAGCCCATCCCCATCTCGAAGGCGCAC |
| | | GAGCACATCTTCGGCATGGTCCTCATGAACGACTGGTCGGCGCGCGACATCCAGAAGTGGGAGTACGTCCCCCTCGGCCCCTT |
| | | CCTCGGCAAGTCGTTCGGCACGACGGTCTCGCCCTGGGTCGTCCCCATGGACGCGCTCATGCCCTTCGCGGTCCCCAACCCCA |
| | | AGCAGGACCCCCGCCCCCTCCCCTACCTCTGCCACGACGAGCCCTACACGTTCGACATCAACCTCTCGGTCAACCTCAAGGGC |
| | | GAGGGCATGTCGCAGGCGGCGACGATCTGCAAGTCGAACTTCAAGTACATGTACTGGACGATGCTCCAGCAGCTCACGCACC |
| | | ACTCGGTCAACGGCTGCAACCTCCGCCCCGGCGACCTCCTCGCGTCGGGCACGATCTCGGGCCCCGAGCCCGAGAACTTCGGC |
| | | TCGATGCTCGAGCTCTCGTGGAAGGGCACGAAGCCCATCGACCTCGGCAACGGCCAGACGCGCAAGTTCCTCCTCGACGGCG |
| | | ACGAGGTCATCATCACGGGCTACTGCCAGGGCGACGGCTACCGCATCGGCTTCGGCCAGTGCGCGGGCAAGGTCCTCCCCGCG |
| | | CTCCTCCCCTAG |
| 91 | FAH | ATGTCGTTCATCCCCGTCGCGGAGGACTCGGACTTCCCCATCCACAATCTTCCATACGGCGTCTTCTCGACTCGCGGCGACCCC |
| | GP_BP_BS_GCU | CGCCCCCGCATCGGCGTCGCGATCGGCGACCAGATCCTCGACCTCAGCATCATCAAGCACCTCTTCACGGGCCCCGTCCTCAG |
| | ORF | CAAGCACCAGGACGTCTTCAACCAGCCCACCCTCAACTCGTTCATGGGCCTCGGCCAGGCTGCCTGGAAGGAAGCCCGCGTCT |
| | | TCCTGCAGAATCTCCTCAGCGTCAGCCAGGCCCGCCTGCGAGACGACACAGAGCTGCGGAAGTGCGCGTTCATCTCGCAGGCC |
| | | TCGGCGACGATGCACCTTCCAGCGACGATCGGCGACTACACGGACTTCTACTCGTCGCGCCAGCACGCGACGAACGTTGGCAT |
| | | CATGTTCCGAGACAAGGAGAACGCACTGATGCCCAACTGGCTCCACCTTCCAGTTGGCTACCACGGCCGCGCGTCGTCGGTCG |
| | | TCGTCAGCGGCACTCCCATCCGCCGCCCCATGGGCCAGATGAAGCCTGACGACTCGAAGCCACCCGTCTACGGTGCCTGCAAG |
| | | CTCCTCGACATGGAGCTGGAGATGGCGTTCTTCGTTGGCCCTGGCAACCGCCTCGGTGAGCCCATCCCCATCTCGAAGGCGCA |
| | | CGAGCACATCTTCGGCATGGTCCTCATGAACGACTGGTCGGCCCGAGACATCCAGAAGTGGGAGTACGTTCCCCTCGGCCCCT |
| | | TCCTGGGCAAGTCGTTCGGCACGACGGTCAGCCCCTGGGTCGTTCCCATGGACGCACTGATGCCCTTCGCTGTTCCCAACCCCA |
| | | AGCAGGACCCCCGCCCCCTTCCATACCTCTGCCACGACGAGCCCTACACGTTCGACATCAATCTCAGCGTCAATCTCAAGGGT |
| | | GAGGGCATGTCGCAGGCTGCCACGATCTGCAAGTCGAACTTCAAGTACATGTACTGGACGATGCTCCAGCAGCTCACGCACCA |
| | | CTCGGTCAATGGGTGCAATCTGCGGCCTGGCGACCTCCTCGCGTCGGGCACGATCTCGGGCCCAGAGCCAGAGAACTTCGGCT |
| | | CGATGCTCGAGCTCAGCTGGAAGGGGACGAAGCCCATCGACCTCGGCAATGGGCAGACGCGCAAGTTCCTGCTCGACGGCGA |
| | | CGAAGTCATCATCACGGGCTACTGCCAGGGCGACGGCTACCGCATCGGCTTCGGCCAGTGCGCCGGGAAGGTCCTTCCAGCAC |
| | | TGCTTCCCTCATAG |
| 92 | FAH | ATGAGCTTCATCCCCGTCGCGGAGGACAGCGACTTCCCCATCCACAATCTTCCATACGGCGTCTTCTCGACTCGCGGCGACCCC |
| | GS BS_GCU | CGCCCCCGCATCGGCGTCGCGATCGGCGACCAGATCCTCGACCTCAGCATCATCAAGCACCTCTTCACGGGCCCCGTCCTCAG |
| | ORF | CAAGCACCAGGACGTCTTCAACCAGCCCACCCTCAACAGCTTCATGGGCCTCGGCCAGGCTGCCTGGAAGGAAGCCCGCGTCT |
| | | TCCTGCAGAATCTCCTCAGCGTCAGCCAGGCCCGCCTGCGAGACGACACAGAGCTGCGGAAGTGCGCGTTCATCAGCCAGGCC |
| | | AGCGCGACGATGCACCTTCCAGCGACGATCGGCGACTACACGGACTTCTACAGCAGCCGCCAGCACGCGACGAACGTTGGCA |
| | | TCATGTTCCGAGACAAGGAGAACGCACTGATGCCCAACTGGCTCCACCTTCCAGTTGGCTACCACGGCCGCGCGAGCAGCGTC |
| | | GTCGTCAGCGGCACTCCCATCCGCCGCCCCATGGGCCAGATGAAGCCTGACGACAGCAAGCCACCCGTCTACGGTGCCTGCAA |
| | | GCTCCTCGACATGGAGCTGGAGATGGCGTTCTTCGTTGGCCCTGGCAACCGCCTCGGTGAGCCCATCCCCATCAGCAAGGCGC |
| | | ACGAGCACATCTTCGGCATGGTCCTCATGAACGACTGGAGCGCCCGAGACATCCAGAAGTGGGAGTACGTTCCCCTCGGCCCC |
| | | TTCCTGGGCAAGAGCTTCGGCACGACGGTCAGCCCCTGGGTCGTTCCCATGGACGCACTGATGCCCTTCGCTGTTCCCAACCCC |
| | | AAGCAGGACCCCCGCCCCCTTCCATACCTCTGCCACGACGAGCCCTACACGTTCGACATCAATCTCAGCGTCAATCTCAAGGG |
| | | TGAGGGCATGAGCCAGGCTGCCACGATCTGCAAGAGCAACTTCAAGTACATGTACTGGACGATGCTCCAGCAGCTCACGCAC |
| | | CACAGCGTCAATGGGTGCAATCTGCGGCCTGGCGACCTCCTCGCGAGCGGCACGATCAGCGGCCCAGAGCCAGAGAACTTCG |
| | | GCAGCATGCTCGAGCTCAGCTGGAAGGGGACGAAGCCCATCGACCTCGGCAATGGGCAGACGCGCAAGTTCCTGCTCGACGG |
| | | CGACGAAGTCATCATCACGGGCTACTGCCAGGGCGACGGCTACCGCATCGGCTTCGGCCAGTGCGCCGGGAAGGTCCTTCCAG |
| | | CACTGCTTCCCTCATAG |
| 93 | FAH GS_GCU | ATGAGCTTCATCCCCGTGGCGGAGGACAGCGACTTCCCGATCCACAATCTTCCATACGGCGTGTTCTCGACTCGCGGCGACCC |
| | ORF | GCGCCCGCGCATCGGCGTGGCGATCGGCGACCAGATCCTGGACCTCAGCATCATCAAGCACCTGTTCACGGGCCCGGTGCTCA |
| | | GCAAGCACCAGGACGTGTTCAACCAGCCCACCCTGAACAGCTTCATGGGCCTGGGCCAGGCTGCCTGGAAGGAAGCCCGCGT |
| | | GTTCCTGCAGAATCTCCTCAGCGTCAGCCAGGCCCGCCTGCGAGACGACACAGAGCTGCGGAAGTGCGCGTTCATCAGCCAGG |
| | | CCAGCGCGACGATGCACCTTCCAGCGACGATCGGCGACTACACGGACTTCTACAGCAGCCGCCAGCACGCGACGAACGTTGG |
| | | CATCATGTTCCGAGACAAGGAGAACGCACTGATGCCGAACTGGCTGCACCTTCCAGTTGGCTACCACGGCCGCGCGAGCAGC |
| | | GTGGTGGTCAGCGGCACTCCCATCCGCCGCCCGATGGGCCAGATGAAGCCTGACGACAGCAAGCCACCCGTGTACGGTGCCT |
| | | GCAAGCTGCTGGACATGGAGCTGGAGATGGCGTTCTTCGTTGGCCCGGGCAACCGCCTGGGTGAGCCGATCCCCATCAGCAAG |
| | | GCGCACGAGCACATCTTCGGCATGGTGCTGATGAACGACTGGAGCGCCCGAGACATCCAGAAGTGGGAGTACGTTCCCCTGG |
| | | GCCCGTTCCTGGGCAAGAGCTTCGGCACGACGGTCAGCCCGTGGGTGGTTCCCATGGACGCACTGATGCCGTTCGCTGTTCCC |
| | | AACCCGAAGCAGGACCCGCGCCCGCTTCCATACCTGTGCCACGACGAGCCGTACACGTTCGACATCAATCTCAGCGTGAATCT |
| | | CAAGGGTGAGGGCATGAGCCAGGCTGCCACGATCTGCAAGAGCAACTTCAAGTACATGTACTGGACGATGCTGCAGCAGCTG |
| | | ACGCACCACAGCGTGAATGGGTGCAATCTGCGGCCGGGCGACCTGCTGGCGAGCGGCACGATCAGCGGCCCAGAGCCAGAGA |
| | | ACTTCGGCAGCATGCTGGAGCTCAGCTGGAAGGGGACGAAGCCGATCGACCTGGGCAATGGGCAGACGCGCAAGTTCCTGCT |
| | | GGACGGCGACGAAGTCATCATCACGGGCTACTGCCAGGGCGACGGCTACCGCATCGGCTTCGGCCAGTGCGCCGGGAAGGTG |
| | | CTTCCAGCACTGCTTCCCTCATAG |
| 94 | GABRD amino | MSEATPLDRNDSENTGGLISRPHPWDQSPSCVQEDRAMNDIGDYVGSNLEISWLPNLDGLIAGYARNFRPGIGGPPVNVALALEVAS |
| | acid sequence | IDHISEANMEYTMTVFLHQSWRDSRLSYNHTNETLGLDSRFVDKLWLPDTFIVNAKSAWFHDVTVENKLIRLQPDGVILYSIRITSTV |
| | | ACDMDLAKYPMDEQECMLDLESYGYSSEDIVYYWSESQEHIHGLDKLQLAQFTITSYRFTTELMNFKSAGQFPRLSLHFHLRRNRG |
| | | VYIIQSYMPSVLLVAMSWVSFWISQAAVPARVSLGITTVLTMTTLMVSARSSLPRASAIKALDVYFWICYVFVFAALVEYAFAHFNA |
| | | DYRKKQKAKVKVSRPRAEMDVRNAIVLFSLSAAGVTQELAISRRQRRVPGNLMGSYRSVGVETGETKKEGAARSGGQGGIRARLR |
| | | PIDADTIDIYARAVFPAAFAAVNVIYWAAYA |
| 95 | GABRD WT | AUGUCUGAAGCUACUCCUUUAGAUCGUAAUGAUUCUGAAAAUACUGGUGGUUUAAUUUCUCGUCCUCAUCCUUGGGAUCA |
| | ORF | AUCUCCUUCUUGUGUUCAAGAAGAUCGUGCUAUGAAUGAUAUUGGUGAUUAUGUUGGUUCUAAUUUAGAAAUUUCUUGG |
| | | UUACCUAAUUUAGAUGGUUUAAUUGCUGGUUAUGCUCGUAAUUUUCGUCCUGGUAUUGGUGGUCCUCCUGUUAAUGUUG |
| | | CUUUAGCUUUAGAAGUUGCUUCUAUUGAUCAUAUUUCUGAAGCUAAUAUGGAAUAUACUAUGACUGUUUUUUUACAUCA |
| | | AUCUUGGCGUGAUUCUCGUUUAUCUUAUAAUCAUACUAAUGAAACUUUAGGUUUAGAUUCUCGUUUUGUUGAUAAAUUA |
| | | UGGUUACCUGAUACUUUUAUUGUUAAUGCUAAAUCUGCUUGGUUUCAUGAUGUUACUGUUGAAAAUAAAUUAAUUCGUU |
| | | UACAACCUGAUGGUGUUAUUUUAUAUUCUAUUCGUAUUACUUCUACUGUUGCUUGUGAUAUGGAUUUAGCUAAAUAUCC |
| | | UAUGGAUGAACAAGAAUGUAUGUUAGAUUUAGAAUCUUAUGGUUAUUCUUCUGAAGAUAUUGUUUAUUAUUGGUCUGAA |
| | | UCUCAAGAACAUAUUCAUGGUUUAGAUAAAUUACAAUUAGCUCAAUUUACUAUUACUUCUUAUCGUUUUACUACUGAAU |
| | | UAAUGAAUUUUAAAUCUGCUGGUCAAUUUCCUCGUUUAUCUUUACAUUUUCAUUUACGUCGUAAUCGUGGUGUUUAUAU |
| | | UAUUCAAUCUUAUAUGCCUUCUGUUUUAUUAGUUGCUAUGUCUUGGGUUUCUUUUUGGAUUUCUCAAGCUGCUGUUCCU |
| | | GCUCGUGUUUCUUUAGGUAUUACUACUGUUUUAACUAUGACUACUUUAAUGGUUUCUGCUCGUUCUUCUUUACCUCGUGC |
| | | UUCUGCUAUUAAAGCUUUAGAUGUUUAUUUUUGGAUUUGUUAUGUUUUUGUUUUUGCUGCUUUAGUUGAAUAUGCUUUU |
| | | GCUCAUUUUAAUGCUGAUUAUCGUAAAAAACAAAAAGCUAAAGUUAAAGUUUCUCGUCCUCGUGCUGAAAUGGAUGUUC |
| | | GUAAUGCUAUUGUUUUAUUUUCUUUAUCUGCUGCUGGUGUUACUCAAGAAUUAGCUAUUUCUCGUCGUCAACGUCGUGU |
| | | UCCUGGUAAUUUAAUGGGUUCUUAUCGUUCUGUUGGUGUUGAAACUGGUGAAACUAAAAAAGAAGGUGCUGCUCGUUCU |
| | | GGUGGUCAAGGUGGUAUUCGUGCUCGUUUACGUCCUAUUGAUGCUGAUACUAUUGAUAUUUAUGCUCGUGCUGUUUUUC |
| | | CUGCUGCUUUUGCUGCUGUUAAUGUUAUUUAUUGGGCUGCUUAUGCUUAG |
| 96 | GABRD | ATGTCGGAGGCGACGCCCCTCGACCGCAACGACTCGGAGAACACGGGCGGCCTCATCTCGCGCCCCCACCCCTGGGACCAGT |
| | BP_GCU ORF | CGCCCTCGTGCGTCCAGGAGGACCGCGCGATGAACGACATCGGCGACTACGTCGGCTCGAACCTCGAGATCTCGTGGCTCCCC |
| | | AACCTCGACGGCCTCATCGCGGGCTACGCGCGCAACTTCCGCCCCGGCATCGGCGGCCCCCCCGTCAACGTCGCGCTCGCGCT |
| | | CGAGGTCGCGTCGATCGACCACATCTCGGAGGCGAACATGGAGTACACGATGACGGTCTTCCTCCACCAGTCGTGGCGCGACT |
| | | CGCGCCTCTCGTACAACCACACGAACGAGACGCTCGGCCTCGACTCGCGCTTCGTCGACAAGCTCTGGCTCCCCGACACGTTC |
| | | ATCGTCAACGCGAAGTCGGCGTGGTTCCACGACGTCACGGTCGAGAACAAGCTCATCCGCCTCCAGCCCGACGGCGTCATCCT |
| | | CTACTCGATCCGCATCACGTCGACGGTCGCGTGCGACATGGACCTCGCGAAGTACCCCATGGACGAGCAGGAGTGCATGCTCG |
| | | ACCTCGAGTCGTACGGCTACTCGTCGGAGGACATCGTCTACTACTGGTCGGAGTCGCAGGAGCACATCCACGGCCTCGACAAG |
| | | CTCCAGCTCGCGCAGTTCACGATCACGTCGTACCGCTTCACGACGGAGCTCATGAACTTCAAGTCGGCGGGCCAGTTCCCCCG |
| | | CCTCTCGCTCCACTTCCACCTCCGCCGCAACCGCGGCGTCTACATCATCCAGTCGTACATGCCCTCGGTCCTCCTCGTCGCGAT |
| | | GTCGTGGGTCTCGTTCTGGATCTCGCAGGCGGCGGTCCCCGCGCGCGTCTCGCTCGGCATCACGACGGTCCTCACGATGACGA |
| | | CGCTCATGGTCTCGGCGCGCTCGTCGCTCCCCCGCGCGTCGGCGATCAAGGCGCTCGACGTCTACTTCTGGATCTGCTACGTCT |
| | | TCGTCTTCGCGGCGCTCGTCGAGTACGCGTTCGCGCACTTCAACGCGGACTACCGCAAGAAGCAGAAGGCGAAGGTCAAGGT |
| | | CTCGCGCCCCCGCGCGGAGATGGACGTCCGCAACGCGATCGTCCTCTTCTCGCTCTCGGCGGCGGGCGTCACGCAGGAGCTCG |
| | | CGATCTCGCGCCGCCAGCGCCGCGTCCCCGGCAACCTCATGGGCTCGTACCGCTCGGTCGGCGTCGAGACGGGCGAGACGAA |
| | | GAAGGAGGGCGCGGCGCGCTCGGGCGGCCAGGGCGGCATCCGCGCGCGCCTCCGCCCCATCGACGCGGACACGATCGACATC |
| | | TACGCGCGCGCGGTCTTCCCCGCGGCGTTCGCGGCGGTCAACGTCATCTACTGGGCGGCGTACGCGTAG |
| 97 | GABRD | ATGTCGGAAGCCACTCCCCTCGACCGCAACGACTCGGAGAACACGGGTGGCCTCATCTCGCGCCCCCACCCCTGGGACCAGAG |
| | GP_BP_BS_GCU | CCCCTCATGCGTCCAGGAGGACCGCGCGATGAACGACATCGGCGACTACGTTGGCTCGAATCTCGAGATCTCGTGGCTTCCAA |
| | ORF | ATCTCGACGGCCTCATCGCCGGGTACGCCCGCAACTTCCGCCCTGGCATCGGTGGCCCACCCGTCAACGTCGCACTGGCACTG |
| | | GAAGTCGCGAGCATCGACCACATCTCGGAAGCCAACATGGAGTACACGATGACGGTCTTCCTGCACCAGAGCTGGCGAGACT |
| | | CGCGCCTCAGCTACAACCACACGAACGAGACGCTCGGCCTCGACTCGCGCTTCGTCGACAAGCTCTGGCTTCCTGACACGTTC |
| | | ATCGTCAACGCGAAGTCGGCGTGGTTCCACGACGTCACGGTCGAGAACAAGCTCATCCGCCTCCAGCCTGACGGCGTCATCCT |
| | | CTACAGCATCCGCATCACGTCGACTGTCGCGTGCGACATGGACCTCGCGAAGTACCCCATGGACGAGCAGGAGTGCATGCTCG |
| | | ACCTCGAGTCGTACGGCTACTCGTCGGAGGACATCGTCTACTACTGGTCGGAGTCGCAGGAGCACATCCACGGCCTCGACAAG |
| | | CTCCAGCTCGCGCAGTTCACGATCACGTCGTACCGCTTCACGACAGAGCTGATGAACTTCAAGTCGGCCGGGCAGTTCCCCCG |
| | | CCTCAGCCTCCACTTCCACCTGCGGCGCAACCGCGGCGTCTACATCATCCAGAGCTACATGCCCTCAGTCCTCCTCGTCGCGAT |
| | | GTCGTGGGTCAGCTTCTGGATCTCGCAGGCTGCTGTTCCCGCCCGCGTCAGCCTCGGCATCACGACGGTCCTCACGATGACGA |
| | | CGCTCATGGTCAGCGCCCGCTCGTCGCTTCCACGCGCGTCGGCGATCAAGGCACTGGACGTCTACTTCTGGATCTGCTACGTCT |
| | | TCGTCTTCGCTGCACTGGTCGAGTACGCGTTCGCGCACTTCAACGCGGACTACCGCAAGAAGCAGAAGGCGAAGGTCAAGGTC |
| | | AGCCGCCCCCGCGCGGAGATGGACGTCCGCAACGCGATCGTCCTCTTCTCGCTCAGCGCTGCCGGGGTCACTCAGGAGCTGGC |
| | | GATCTCGCGCCGCCAGCGCCGCGTTCCCGGCAATCTCATGGGCTCGTACCGATCGGTTGGCGTCGAGACGGGTGAGACGAAGA |
| | | AGGAGGGTGCTGCCCGCTCGGGTGGCCAGGGTGGCATCCGCGCCCGCCTGCGGCCCATCGACGCGGACACGATCGACATCTA |
| | | CGCCCGCGCTGTGTTCCCTGCTGCCTTCGCTGCTGTGAACGTCATCTACTGGGCTGCCTACGCGTAG |
| 98 | GABRD | ATGAGCGAAGCCACTCCCCTCGACCGCAACGACAGCGAGAACACGGGTGGCCTCATCAGCCGCCCCCACCCCTGGGACCAGA |
| | GS_BS_GCU | GCCCCTCATGCGTCCAGGAGGACCGCGCGATGAACGACATCGGCGACTACGTTGGCAGCAATCTCGAGATCAGCTGGCTTCCA |
| | ORF | AATCTCGACGGCCTCATCGCCGGGTACGCCCGCAACTTCCGCCCTGGCATCGGTGGCCCACCCGTCAACGTCGCACTGGCACT |
| | | GGAAGTCGCGAGCATCGACCACATCAGCGAAGCCAACATGGAGTACACGATGACGGTCTTCCTGCACCAGAGCTGGCGAGAC |
| | | AGCCGCCTCAGCTACAACCACACGAACGAGACGCTCGGCCTCGACAGCCGCTTCGTCGACAAGCTCTGGCTTCCTGACACGTT |
| | | CATCGTCAACGCGAAGAGCGCGTGGTTCCACGACGTCACGGTCGAGAACAAGCTCATCCGCCTCCAGCCTGACGGCGTCATCC |
| | | TCTACAGCATCCGCATCACGTCGACTGTCGCGTGCGACATGGACCTCGCGAAGTACCCCATGGACGAGCAGGAGTGCATGCTC |
| | | GACCTCGAGAGCTACGGCTACAGCAGCGAGGACATCGTCTACTACTGGAGCGAGAGCCAGGAGCACATCCACGGCCTCGACA |
| | | AGCTCCAGCTCGCGCAGTTCACGATCACGAGCTACCGCTTCACGACAGAGCTGATGAACTTCAAGAGCGCCGGGCAGTTCCCC |
| | | CGCCTCAGCCTCCACTTCCACCTGCGGCGCAACCGCGGCGTCTACATCATCCAGAGCTACATGCCCTCAGTCCTCCTCGTCGCG |
| | | ATGAGCTGGGTCAGCTTCTGGATCAGCCAGGCTGCTGTTCCCGCCCGCGTCAGCCTCGGCATCACGACGGTCCTCACGATGAC |
| | | GACGCTCATGGTCAGCGCCCGCAGCAGCCTTCCACGCGCGAGCGCGATCAAGGCACTGGACGTCTACTTCTGGATCTGCTACG |
| | | TCTTCGTCTTCGCTGCACTGGTCGAGTACGCGTTCGCGCACTTCAACGCGGACTACCGCAAGAAGCAGAAGGCGAAGGTCAAG |
| | | GTCAGCCGCCCCCGCGCGGAGATGGACGTCCGCAACGCGATCGTCCTCTTCAGCCTCAGCGCTGCCGGGGTCACTCAGGAGCT |
| | | GGCGATCAGCCGCCGCCAGCGCCGCGTTCCCGGCAATCTCATGGGCAGCTACAGGAGCGTTGGCGTCGAGACGGGTGAGACG |
| | | AAGAAGGAGGGTGCTGCCCGCAGCGGTGGCCAGGGTGGCATCCGCGCCCGCCTGCGGCCCATCGACGCGGACACGATCGACA |
| | | TCTACGCCCGCGCTGTGTTCCCTGCTGCCTTCGCTGCTGTGAACGTCATCTACTGGGCTGCCTACGCGTAG |
| 99 | GABRD | ATGAGCGAAGCCACTCCCCTGGACCGCAACGACAGCGAGAACACGGGTGGCCTGATCAGCCGCCCGCACCCGTGGGACCAGA |
| | GS_GCU ORF | GCCCCTCATGCGTGCAGGAGGACCGCGCGATGAACGACATCGGCGACTACGTTGGCAGCAATCTCGAGATCAGCTGGCTTCCA |
| | | AATCTCGACGGCCTGATCGCCGGGTACGCCCGCAACTTCCGCCCGGGCATCGGTGGCCCACCCGTGAACGTGGCACTGGCACT |
| | | GGAAGTCGCGAGCATCGACCACATCAGCGAAGCCAACATGGAGTACACGATGACGGTGTTCCTGCACCAGAGCTGGCGAGAC |
| | | AGCCGCCTCAGCTACAACCACACGAACGAGACGCTGGGCCTGGACAGCCGCTTCGTGGACAAGCTGTGGCTTCCTGACACGTT |
| | | CATCGTGAACGCGAAGAGCGCGTGGTTCCACGACGTGACGGTGGAGAACAAGCTGATCCGCCTGCAGCCTGACGGCGTGATC |
| | | CTGTACAGCATCCGCATCACGTCGACTGTGGCGTGCGACATGGACCTGGCGAAGTACCCGATGGACGAGCAGGAGTGCATGC |
| | | TGGACCTGGAGAGCTACGGCTACAGCAGCGAGGACATCGTGTACTACTGGAGCGAGAGCCAGGAGCACATCCACGGCCTGGA |
| | | CAAGCTGCAGCTGGCGCAGTTCACGATCACGAGCTACCGCTTCACGACAGAGCTGATGAACTTCAAGAGCGCCGGGCAGTTCC |
| | | CGCGCCTCAGCCTGCACTTCCACCTGCGGCGCAACCGCGGCGTGTACATCATCCAGAGCTACATGCCCTCAGTGCTGCTGGTG |
| | | GCGATGAGCTGGGTCAGCTTCTGGATCAGCCAGGCTGCTGTTCCCGCCCGCGTCAGCCTGGGCATCACGACGGTGCTGACGAT |
| | | GACGACGCTGATGGTCAGCGCCCGCAGCAGCCTTCCACGCGCGAGCGCGATCAAGGCACTGGACGTGTACTTCTGGATCTGCT |
| | | ACGTGTTCGTGITCGCTGCACTGGTGGAGTACGCGTTCGCGCACTTCAACGCGGACTACCGCAAGAAGCAGAAGGCGAAGGT |
| | | GAAGGTCAGCCGCCCGCGCGCGGAGATGGACGTGCGCAACGCGATCGTGCTGTTCAGCCTCAGCGCTGCCGGGGTGACTCAG |
| | | GAGCTGGCGATCAGCCGCCGCCAGCGCCGCGTTCCCGGCAATCTCATGGGCAGCTACCGCAGCGTTGGCGTGGAGACGGGTG |
| | | AGACGAAGAAGGAGGGTGCTGCCCGCAGCGGTGGCCAGGGTGGCATCCGCGCCCGCCTGCGGCCGATCGACGCGGACACGAT |
| | | CGACATCTACGCCCGCGCTGTGTTCCCGGCTGCCITCGCTGCTGTGAACGTGATCTACTGGGCTGCCTACGCGTAG |
| 100 | GAPDH amino | MGKVKVGVNGFGRIGRLVTRAAFNSGKVDIVAINDPFIDLNYMAENGKLVINGNPITIFQERDPSKIKWGDAGAEYVVESTGVFTT |
| | acid sequence | MEKAGAHLQGGAKRVIISAPSADAPMFVMGVNHEKYDNSLKIISNASCTTNCLAPLAKVIHDNFGIVEGLMTTVHAITATQKTVDG |
| | | PSGKLWRDGRGALQNIIPASTGAAKAVGKVIPELNGKLTGMAFRVPTANVSVVDLTCRLEKPAKYDDIKKVVKQASEGPLKGILGY |
| | | TEHQVVSSDFNSDTHSSTFDAGAGIALNDHFVKLISWYDNEFGYSNRVVDLMAHMASK |
| 101 | GAPDH WT | AUGGGUAAAGUUAAAGUUGGUGUUAAUGGUUUUGGUCGUAUUGGUCGUUUAGUUACUCGUGCUGCUUUUAAUUCUGGUA |
| | ORF | AAGUUGAUAUUGUUGCUAUUAAUGAUCCUUUUAUUGAUUUAAAUUAUAUGGCUGAAAAUGGUAAAUUAGUUAUUAAUGG |
| | | UAAUCCUAUUACUAUUUUUCAAGAACGUGAUCCUUCUAAAAUUAAAUGGGGUGAUGCUGGUGCUGAAUAUGUUGUUGAA |
| | | UCUACUGGUGUUUUUACUACUAUGGAAAAAGCUGGUGCUCAUUUACAAGGUGGUGCUAAACGUGUUAUUAUUUCUGCUC |
| | | CUUCUGCUGAUGCUCCUAUGUUUGUUAUGGGUGUUAAUCAUGAAAAAUAUGAUAAUUCUUUAAAAAUUAUUUCUAAUGC |
| | | UUCUUGUACUACUAAUUGUUUAGCUCCUUUAGCUAAAGUUAUUCAUGAUAAUUUUGGUAUUGUUGAAGGUUUAAUGACU |
| | | ACUGUUCAUGCUAUUACUGCUACUCAAAAAACUGUUGAUGGUCCUUCUGGUAAAUUAUGGCGUGAUGGUCGUGGUGCUU |
| | | UACAAAAUAUUAUUCCUGCUUCUACUGGUGCUGCUAAAGCUGUUGGUAAAGUUAUUCCUGAAUUAAAUGGUAAAUUAAC |
| | | UGGUAUGGCUUUUCGUGUUCCUACUGCUAAUGUUUCUGUUGUUGAUUUAACUUGUCGUUUAGAAAAACCUGCUAAAUAU |
| | | GAUGAUAUUAAAAAAGUUGUUAAACAAGCUUCUGAAGGUCCUUUAAAAGGUAUUUUAGGUUAUACUGAACAUCAAGUUG |
| | | UUUCUUCUGAUUUUAAUUCUGAUACUCAUUCUUCUACUUUUGAUGCUGGUGCUGGUAUUGCUUUAAAUGAUCAUUUUGU |
| | | UAAAUUAAUUUCUUGGUAUGAUAAUGAAUUUGGUUAUUCUAAUCGUGUUGUUGAUUUAAUGGCUCAUAUGGCUUCUAAA |
| | | UAG |
| 102 | GAPDH | ATGGGCAAGGTCAAGGTCGGCGTCAACGGCTTCGGCCGCATCGGCCGCCTCGTCACGCGCGCGGCGTTCAACTCGGGCAAGG |
| | BP_GCU ORF | TCGACATCGTCGCGATCAACGACCCCTTCATCGACCTCAACTACATGGCGGAGAACGGCAAGCTCGTCATCAACGGCAACCCC |
| | | ATCACGATCTTCCAGGAGCGCGACCCCTCGAAGATCAAGTGGGGCGACGCGGGCGCGGAGTACGTCGTCGAGTCGACGGGCG |
| | | TCTTCACGACGATGGAGAAGGCGGGCGCGCACCTCCAGGGCGGCGCGAAGCGCGTCATCATCTCGGCGCCCTCGGCGGACGC |
| | | GCCCATGTTCGTCATGGGCGTCAACCACGAGAAGTACGACAACTCGCTCAAGATCATCTCGAACGCGTCGTGCACGACGAACT |
| | | GCCTCGCGCCCCTCGCGAAGGTCATCCACGACAACTTCGGCATCGTCGAGGGCCTCATGACGACGGTCCACGCGATCACGGCG |
| | | ACGCAGAAGACGGTCGACGGCCCCTCGGGCAAGCTCTGGCGCGACGGCCGCGGCGCGCTCCAGAACATCATCCCCGCGTCGA |
| | | CGGGCGCGGCGAAGGCGGTCGGCAAGGTCATCCCCGAGCTCAACGGCAAGCTCACGGGCATGGCGTTCCGCGTCCCCACGGC |
| | | GAACGTCTCGGTCGTCGACCTCACGTGCCGCCTCGAGAAGCCCGCGAAGTACGACGACATCAAGAAGGTCGTCAAGCAGGCG |
| | | TCGGAGGGCCCCCTCAAGGGCATCCTCGGCTACACGGAGCACCAGGTCGTCTCGTCGGACTTCAACTCGGACACGCACTCGTC |
| | | GACGTTCGACGCGGGCGCGGGCATCGCGCTCAACGACCACTTCGTCAAGCTCATCTCGTGGTACGACAACGAGTTCGGCTACT |
| | | CGAACCGCGTCGTCGACCTCATGGCGCACATGGCGTCGAAGTAG |
| 103 | GAPDH | ATGGGCAAGGTCAAGGTTGGCGTCAATGGGTTCGGCCGCATCGGCCGCCTCGTCACGCGCGCTGCCTTCAACTCGGGCAAGGT |
| | GP_BP_BS_GCU | CGACATCGTCGCGATCAACGACCCCTTCATCGACCTCAACTACATGGCGGAGAATGGGAAGCTCGTCATCAATGGGAACCCCA |
| | ORF | TCACGATCTTCCAGGAGCGAGACCCCTCAAAGATCAAGTGGGGCGACGCCGGTGCCGAGTACGTCGTCGAGTCGACTGGCGT |
| | | CTTCACGACGATGGAGAAGGCCGGTGCCCACCTCCAGGGTGGTGCCAAGCGCGTCATCATCTCGGCGCCCTCAGCGGACGCGC |
| | | CCATGTTCGTCATGGGCGTCAACCACGAGAAGTACGACAACTCGCTCAAGATCATCTCGAACGCGTCGTGCACGACGAACTGC |
| | | CTCGCGCCCCTCGCGAAGGTCATCCACGACAACTTCGGCATCGTCGAGGGCCTCATGACGACGGTCCACGCGATCACGGCGAC |
| | | TCAAAAGACGGTCGACGGCCCCTCAGGCAAGCTCTGGCGAGACGGCCGCGGTGCACTGCAGAACATCATCCCCGCGTCGACT |
| | | GGTGCTGCCAAGGCTGTTGGCAAGGTCATCCCAGAGCTGAATGGGAAGCTCACGGGCATGGCGTTCCGCGTTCCCACCGCGAA |
| | | CGTCAGCGTCGTCGACCTCACGTGCCGCCTCGAGAAGCCTGCGAAGTACGACGACATCAAGAAGGTCGTCAAGCAGGCCTCG |
| | | GAGGGCCCCCTCAAGGGGATCCTCGGCTACACAGAGCACCAGGTGGTCAGCTCGGACTTCAACTCGGACACGCACTCGTCGA |
| | | CTTTCGACGCCGGTGCCGGGATCGCACTGAACGACCACTTCGTCAAGCTCATCTCGTGGTACGACAACGAGTTCGGCTACTCG |
| | | AACCGCGTCGTCGACCTCATGGCGCACATGGCGTCGAAGTAG |
| 104 | GAPDH | ATGGGCAAGGTCAAGGTTGGCGTCAATGGGTTCGGCCGCATCGGCCGCCTCGTCACGCGCGCTGCCTTCAACAGCGGCAAGGT |
| | GS_BS_GCU | CGACATCGTCGCGATCAACGACCCCTTCATCGACCTCAACTACATGGCGGAGAATGGGAAGCTCGTCATCAATGGGAACCCCA |
| | ORF | TCACGATCTTCCAGGAGCGAGACCCCTCAAAGATCAAGTGGGGCGACGCCGGTGCCGAGTACGTCGTCGAGTCGACTGGCGT |
| | | CTTCACGACGATGGAGAAGGCCGGTGCCCACCTCCAGGGTGGTGCCAAGCGCGTCATCATCAGCGCGCCCTCAGCGGACGCG |
| | | CCCATGTTCGTCATGGGCGTCAACCACGAGAAGTACGACAACAGCCTCAAGATCATCAGCAACGCGAGCTGCACGACGAACT |
| | | GCCTCGCGCCCCTCGCGAAGGTCATCCACGACAACTTCGGCATCGTCGAGGGCCTCATGACGACGGTCCACGCGATCACGGCG |
| | | ACTCAAAAGACGGTCGACGGCCCCTCAGGCAAGCTCTGGCGAGACGGCCGCGGTGCACTGCAGAACATCATCCCCGCGTCGA |
| | | CTGGTGCTGCCAAGGCTGTTGGCAAGGTCATCCCAGAGCTGAATGGGAAGCTCACGGGCATGGCGTTCCGCGTTCCCACCGCG |
| | | AACGTCAGCGTCGTCGACCTCACGTGCCGCCTCGAGAAGCCTGCGAAGTACGACGACATCAAGAAGGTCGTCAAGCAGGCCA |
| | | GCGAGGGCCCCCTCAAGGGGATCCTCGGCTACACAGAGCACCAGGTGGTCAGCAGCGACTTCAACAGCGACACGCACAGCTC |
| | | GACTTTCGACGCCGGTGCCGGGATCGCACTGAACGACCACTTCGTCAAGCTCATCAGCTGGTACGACAACGAGTTCGGCTACA |
| | | GCAACCGCGTCGTCGACCTCATGGCGCACATGGCGAGCAAGTAG |
| 105 | GAPDH | ATGGGCAAGGTGAAGGTTGGCGTGAATGGGTTCGGCCGCATCGGCCGCCTGGTGACGCGCGCTGCCTTCAACAGCGGCAAGG |
| | GS_GCU ORF | TGGACATCGTGGCGATCAACGACCCGTTCATCGACCTGAACTACATGGCGGAGAATGGGAAGCTGGTGATCAATGGGAACCC |
| | | GATCACGATCTTCCAGGAGCGAGACCCCTCAAAGATCAAGTGGGGCGACGCCGGTGCCGAGTACGTGGTGGAGTCGACTGGC |
| | | GTGTTCACGACGATGGAGAAGGCCGGTGCCCACCTGCAGGGTGGTGCCAAGCGCGTGATCATCAGCGCGCCCTCAGCGGACG |
| | | CGCCGATGTTCGTGATGGGCGTGAACCACGAGAAGTACGACAACAGCCTGAAGATCATCAGCAACGCGAGCTGCACGACGAA |
| | | CTGCCTGGCGCCGCTGGCGAAGGTGATCCACGACAACTTCGGCATCGTGGAGGGCCTGATGACGACGGTGCACGCGATCACG |
| | | GCGACTCAAAAGACGGTGGACGGCCCCTCAGGCAAGCTGTGGCGAGACGGCCGCGGTGCACTGCAGAACATCATCCCCGCGT |
| | | CGACTGGTGCTGCCAAGGCTGTTGGCAAGGTGATCCCAGAGCTGAATGGGAAGCTGACGGGCATGGCGTTCCGCGTTCCCACC |
| | | GCGAACGTCAGCGTGGTGGACCTGACGTGCCGCCTGGAGAAGCCGGCGAAGTACGACGACATCAAGAAGGTGGTGAAGCAG |
| | | GCCAGCGAGGGCCCGCTGAAGGGGATCCTGGGCTACACAGAGCACCAGGTGGTCAGCAGCGACTTCAACAGCGACACGCACA |
| | | GCTCGACTITCGACGCCGGTGCCGGGATCGCACTGAACGACCACTTCGTGAAGCTGATCAGCTGGTACGACAACGAGTTCGGC |
| | | TACAGCAACCGCGTGGTGGACCTGATGGCGCACATGGCGAGCAAGTAG |
| 106 | GBA1 amino | MEFSSPSREECPKPLSRVSIMAGSLTGLLLLQAVSWASGARPCIPKSFGYSSVVCVCNATYCDSFDPPTFPALGTFSRYESTRSGRRM |
| | acid sequence | ELSMGPIQANHTGTGLLLTLQPEQKFQKVKGFGGAMTDAAALNILALSPPAQNLLLKSYFSEEGIGYNIIRVPMASCDFSIRTYTYAD |
| | | TPDDFQLHNFSLPEEDTKLKIPLIHRALQLAQRPVSLLASPWTSPTWLKTNGAVNGKGSLKGQPGDIYHQTWARYFVKFLDAYAEH |
| | | KLQFWAVTAENEPSAGLLSGYPFQCLGFTPEHQRDFIARDLGPTLANSTHHNVRLLMLDDQRLLLPHWAKVVLTDPEAAKYVHGI |
| | | AVHWYLDFLAPAKATLGETHRLFPNTMLFASEACVGSKFWEQSVRLGSWDRGMQYSHSIITNLLYHVVGWTDWNLALNPEGGPN |
| | | WVRNFVDSPIIVDITKDTFYKQPMFYHLGHFSKFIPEGSQRVGLVASQKNDLDAVALMHPDGSAVVVVLNRSSKDVPLTIKDPAVG |
| | | FLETISPGYSIHTYLWRR |
| 107 | GBA1 WT ORF | AUGGAAUUUUCUUCUCCUUCUCGUGAAGAAUGUCCUAAACCUUUAUCUCGUGUUUCUAUUAUGGCUGGUUCUUUAACUGG |
| | | UUUAUUAUUAUUACAAGCUGUUUCUUGGGCUUCUGGUGCUCGUCCUUGUAUUCCUAAAUCUUUUGGUUAUUCUUCUGUU |
| | | GUUUGUGUUUGUAAUGCUACUUAUUGUGAUUCUUUUGAUCCUCCUACUUUUCCUGCUUUAGGUACUUUUUCUCGUUAUG |
| | | AAUCUACUCGUUCUGGUCGUCGUAUGGAAUUAUCUAUGGGUCCUAUUCAAGCUAAUCAUACUGGUACUGGUUUAUUAUU |
| | | AACUUUACAACCUGAACAAAAAUUUCAAAAAGUUAAAGGUUUUGGUGGUGCUAUGACUGAUGCUGCUGCUUUAAAUAUU |
| | | UUAGCUUUAUCUCCUCCUGCUCAAAAUUUAUUAUUAAAAUCUUAUUUUUCUGAAGAAGGUAUUGGUUAUAAUAUUAUUC |
| | | GUGUUCCUAUGGCUUCUUGUGAUUUUUCUAUUCGUACUUAUACUUAUGCUGAUACUCCUGAUGAUUUUCAAUUACAUAA |
| | | UUUUUCUUUACCUGAAGAAGAUACUAAAUUAAAAAUUCCUUUAAUUCAUCGUGCUUUACAAUUAGCUCAACGUCCUGUUU |
| | | CUUUAUUAGCUUCUCCUUGGACUUCUCCUACUUGGUUAAAAACUAAUGGUGCUGUUAAUGGUAAAGGUUCUUUAAAAGG |
| | | UCAACCUGGUGAUAUUUAUCAUCAAACUUGGGCUCGUUAUUUUGUUAAAUUUUUAGAUGCUUAUGCUGAACAUAAAUUA |
| | | CAAUUUUGGGCUGUUACUGCUGAAAAUGAACCUUCUGCUGGUUUAUUAUCUGGUUAUCCUUUUCAAUGUUUAGGUUUUA |
| | | CUCCUGAACAUCAACGUGAUUUUAUUGCUCGUGAUUUAGGUCCUACUUUAGCUAAUUCUACUCAUCAUAAUGUUCGUUUA |
| | | UUAAUGUUAGAUGAUCAACGUUUAUUAUUACCUCAUUGGGCUAAAGUUGUUUUAACUGAUCCUGAAGCUGCUAAAUAUG |
| | | UUCAUGGUAUUGCUGUUCAUUGGUAUUUAGAUUUUUUAGCUCCUGCUAAAGCUACUUUAGGUGAAACUCAUCGUUUAUU |
| | | UCCUAAUACUAUGUUAUUUGCUUCUGAAGCUUGUGUUGGUUCUAAAUUUUGGGAACAAUCUGUUCGUUUAGGUUCUUGG |
| | | GAUCGUGGUAUGCAAUAUUCUCAUUCUAUUAUUACUAAUUUAUUAUAUCAUGUUGUUGGUUGGACUGAUUGGAAUUUAG |
| | | CUUUAAAUCCUGAAGGUGGUCCUAAUUGGGUUCGUAAUUUUGUUGAUUCUCCUAUUAUUGUUGAUAUUACUAAAGAUAC |
| | | UUUUUAUAAACAACCUAUGUUUUAUCAUUUAGGUCAUUUUUCUAAAUUUAUUCCUGAAGGUUCUCAACGUGUUGGUUUA |
| | | GUUGCUUCUCAAAAAAAUGAUUUAGAUGCUGUUGCUUUAAUGCAUCCUGAUGGUUCUGCUGUUGUUGUUGUUUUAAAUC |
| | | GUUCUUCUAAAGAUGUUCCUUUAACUAUUAAAGAUCCUGCUGUUGGUUUUUUAGAAACUAUUUCUCCUGGUUAUUCUAU |
| | | UCAUACUUAUUUAUGGCGUCGUUAG |
| 108 | GBA1 BP_GCU | ATGGAGTTCTCGTCGCCCTCGCGCGAGGAGTGCCCCAAGCCCCTCTCGCGCGTCTCGATCATGGCGGGCTCGCTCACGGGCCT |
| | ORF | CCTCCTCCTCCAGGCGGTCTCGTGGGCGTCGGGCGCGCGCCCCTGCATCCCCAAGTCGTTCGGCTACTCGTCGGTCGTCTGCGT |
| | | CTGCAACGCGACGTACTGCGACTCGTTCGACCCCCCCACGTTCCCCGCGCTCGGCACGTTCTCGCGCTACGAGTCGACGCGCTC |
| | | GGGCCGCCGCATGGAGCTCTCGATGGGCCCCATCCAGGCGAACCACACGGGCACGGGCCTCCTCCTCACGCTCCAGCCCGAGC |
| | | AGAAGTTCCAGAAGGTCAAGGGCTTCGGCGGCGCGATGACGGACGCGGCGGCGCTCAACATCCTCGCGCTCTCGCCCCCCGC |
| | | GCAGAACCTCCTCCTCAAGTCGTACTTCTCGGAGGAGGGCATCGGCTACAACATCATCCGCGTCCCCATGGCGTCGTGCGACT |
| | | TCTCGATCCGCACGTACACGTACGCGGACACGCCCGACGACTTCCAGCTCCACAACTTCTCGCTCCCCGAGGAGGACACGAAG |
| | | CTCAAGATCCCCCTCATCCACCGCGCGCTCCAGCTCGCGCAGCGCCCCGTCTCGCTCCTCGCGTCGCCCTGGACGTCGCCCACG |
| | | TGGCTCAAGACGAACGGCGCGGTCAACGGCAAGGGCTCGCTCAAGGGCCAGCCCGGCGACATCTACCACCAGACGTGGGCGC |
| | | GCTACTTCGTCAAGTTCCTCGACGCGTACGCGGAGCACAAGCTCCAGTTCTGGGCGGTCACGGCGGAGAACGAGCCCTCGGCG |
| | | GGCCTCCTCTCGGGCTACCCCTTCCAGTGCCTCGGCTTCACGCCCGAGCACCAGCGCGACTTCATCGCGCGCGACCTCGGCCCC |
| | | ACGCTCGCGAACTCGACGCACCACAACGTCCGCCTCCTCATGCTCGACGACCAGCGCCTCCTCCTCCCCCACTGGGCGAAGGT |
| | | CGTCCTCACGGACCCCGAGGCGGCGAAGTACGTCCACGGCATCGCGGTCCACTGGTACCTCGACTTCCTCGCGCCCGCGAAGG |
| | | CGACGCTCGGCGAGACGCACCGCCTCTTCCCCAACACGATGCTCTTCGCGTCGGAGGCGTGCGTCGGCTCGAAGTTCTGGGAG |
| | | CAGTCGGTCCGCCTCGGCTCGTGGGACCGCGGCATGCAGTACTCGCACTCGATCATCACGAACCTCCTCTACCACGTCGTCGG |
| | | CTGGACGGACTGGAACCTCGCGCTCAACCCCGAGGGCGGCCCCAACTGGGTCCGCAACTTCGTCGACTCGCCCATCATCGTCG |
| | | ACATCACGAAGGACACGTTCTACAAGCAGCCCATGTTCTACCACCTCGGCCACTTCTCGAAGTTCATCCCCGAGGGCTCGCAG |
| | | CGCGTCGGCCTCGTCGCGTCGCAGAAGAACGACCTCGACGCGGTCGCGCTCATGCACCCCGACGGCTCGGCGGTCGTCGTCGT |
| | | CCTCAACCGCTCGTCGAAGGACGTCCCCCTCACGATCAAGGACCCCGCGGTCGGCTTCCTCGAGACGATCTCGCCCGGCTACT |
| | | CGATCCACACGTACCTCTGGCGCCGCTAG |
| 109 | GBA1 | ATGGAGTTCTCGTCGCCCTCAAGGGAGGAGTGCCCCAAGCCCCTCAGCCGCGTCAGCATCATGGCCGGGTCGCTCACGGGCCT |
| | GP_BP_BS_GCU | CCTCCTCCTCCAGGCTGTCAGCTGGGCGTCGGGTGCCCGCCCCTGCATCCCCAAGTCGTTCGGCTACTCGTCGGTCGTCTGCGT |
| | ORF | CTGCAACGCGACCTACTGCGACTCGTTCGACCCACCCACCTTCCCTGCACTGGGCACGTTCTCGCGCTACGAGTCGACTCGATC |
| | | GGGCCGCCGCATGGAGCTCAGCATGGGCCCCATCCAGGCCAACCACACGGGCACGGGCCTCCTCCTCACGCTCCAGCCAGAG |
| | | CAGAAGTTCCAGAAGGTCAAGGGGTTCGGTGGTGCCATGACGGACGCTGCTGCACTGAACATCCTCGCACTCAGCCCACCCGC |
| | | GCAGAATCTCCTCCTCAAGTCGTACTTCTCGGAGGAGGGCATCGGCTACAACATCATCCGCGTTCCCATGGCGTCGTGCGACTT |
| | | CAGCATCCGCACCTACACCTACGCGGACACTCCTGACGACTTCCAGCTCCACAACTTCTCGCTTCCAGAGGAGGACACGAAGC |
| | | TCAAGATCCCCCTCATCCACCGCGCACTGCAGCTCGCGCAGCGCCCCGTCAGCCTCCTCGCGTCGCCCTGGACGTCGCCCACCT |
| | | GGCTCAAGACGAATGGTGCTGTGAATGGGAAGGGGTCGCTCAAGGGGCAGCCCGGCGACATCTACCACCAGACGTGGGCCCG |
| | | CTACTTCGTCAAGTTCCTGGACGCGTACGCGGAGCACAAGCTCCAGTTCTGGGCTGTGACGGCGGAGAACGAGCCCTCAGCCG |
| | | GGCTCCTCAGCGGCTACCCCTTCCAGTGCCTCGGCTTCACTCCAGAGCACCAGCGAGACTTCATCGCCCGAGACCTCGGCCCC |
| | | ACCCTCGCGAACTCGACTCACCACAACGTCCGCCTCCTCATGCTCGACGACCAGCGCCTCCTCCTTCCACACTGGGCGAAGGT |
| | | CGTCCTCACGGACCCAGAAGCTGCCAAGTACGTCCACGGCATCGCTGTGCACTGGTACCTCGACTTCCTGGCGCCTGCGAAGG |
| | | CGACGCTCGGTGAGACGCACCGCCTCTTCCCCAACACGATGCTCTTCGCGTCGGAAGCCTGCGTTGGCTCGAAGTTCTGGGAG |
| | | CAGAGCGTCCGCCTCGGCTCGTGGGACCGCGGCATGCAGTACTCGCACAGCATCATCACGAATCTCCTCTACCACGTCGTTGG |
| | | CTGGACGGACTGGAATCTCGCACTGAACCCAGAGGGTGGCCCCAACTGGGTCCGCAACTTCGTCGACTCGCCCATCATCGTCG |
| | | ACATCACGAAGGACACGTTCTACAAGCAGCCCATGTTCTACCACCTCGGCCACTTCTCGAAGTTCATCCCAGAGGGCTCGCAG |
| | | CGCGTTGGCCTCGTCGCGTCGCAGAAGAACGACCTCGACGCTGTGGCACTGATGCACCCTGACGGCTCGGCTGTGGTCGTCGT |
| | | CCTCAACCGATCGTCGAAGGACGTTCCCCTCACGATCAAGGACCCTGCTGTTGGCTTCCTGGAGACGATCTCGCCTGGCTACA |
| | | GCATCCACACCTACCTCTGGCGCCGCTAG |
| 110 | GBA1 | ATGGAGTTCAGCAGCCCCTCAAGGGAGGAGTGCCCCAAGCCCCTCAGCCGCGTCAGCATCATGGCCGGGAGCCTCACGGGCC |
| | GS_BS_GCU | TCCTCCTCCTCCAGGCTGTCAGCTGGGCGAGCGGTGCCCGCCCCTGCATCCCCAAGAGCTTCGGCTACAGCAGCGTCGTCTGC |
| | ORF | GTCTGCAACGCGACCTACTGCGACAGCTTCGACCCACCCACCTTCCCTGCACTGGGCACGTTCAGCCGCTACGAGTCGACTAG |
| | | GAGCGGCCGCCGCATGGAGCTCAGCATGGGCCCCATCCAGGCCAACCACACGGGCACGGGCCTCCTCCTCACGCTCCAGCCA |
| | | GAGCAGAAGTTCCAGAAGGTCAAGGGGTTCGGTGGTGCCATGACGGACGCTGCTGCACTGAACATCCTCGCACTCAGCCCAC |
| | | CCGCGCAGAATCTCCTCCTCAAGAGCTACTTCAGCGAGGAGGGCATCGGCTACAACATCATCCGCGTTCCCATGGCGAGCTGC |
| | | GACTTCAGCATCCGCACCTACACCTACGCGGACACTCCTGACGACTTCCAGCTCCACAACTTCAGCCTTCCAGAGGAGGACAC |
| | | GAAGCTCAAGATCCCCCTCATCCACCGCGCACTGCAGCTCGCGCAGCGCCCCGTCAGCCTCCTCGCGAGCCCCTGGACGAGCC |
| | | CCACCTGGCTCAAGACGAATGGTGCTGTGAATGGGAAGGGGAGCCTCAAGGGGCAGCCCGGCGACATCTACCACCAGACGTG |
| | | GGCCCGCTACTTCGTCAAGTTCCTGGACGCGTACGCGGAGCACAAGCTCCAGTTCTGGGCTGTGACGGCGGAGAACGAGCCCT |
| | | CAGCCGGGCTCCTCAGCGGCTACCCCTTCCAGTGCCTCGGCTTCACTCCAGAGCACCAGCGAGACTTCATCGCCCGAGACCTC |
| | | GGCCCCACCCTCGCGAACTCGACTCACCACAACGTCCGCCTCCTCATGCTCGACGACCAGCGCCTCCTCCTTCCACACTGGGCG |
| | | AAGGTCGTCCTCACGGACCCAGAAGCTGCCAAGTACGTCCACGGCATCGCTGTGCACTGGTACCTCGACTTCCTGGCGCCTGC |
| | | GAAGGCGACGCTCGGTGAGACGCACCGCCTCTTCCCCAACACGATGCTCTTCGCGAGCGAAGCCTGCGTTGGCAGCAAGTTCT |
| | | GGGAGCAGAGCGTCCGCCTCGGCAGCTGGGACCGCGGCATGCAGTACAGCCACAGCATCATCACGAATCTCCTCTACCACGTC |
| | | GTTGGCTGGACGGACTGGAATCTCGCACTGAACCCAGAGGGTGGCCCCAACTGGGTCCGCAACTTCGTCGACAGCCCCATCAT |
| | | CGTCGACATCACGAAGGACACGTTCTACAAGCAGCCCATGTTCTACCACCTCGGCCACTTCAGCAAGTTCATCCCAGAGGGCA |
| | | GCCAGCGCGTTGGCCTCGTCGCGAGCCAGAAGAACGACCTCGACGCTGTGGCACTGATGCACCCTGACGGCAGCGCTGTGGTC |
| | | GTCGTCCTCAACAGGAGCAGCAAGGACGTTCCCCTCACGATCAAGGACCCTGCTGTTGGCTTCCTGGAGACGATCAGCCCTGG |
| | | CTACAGCATCCACACCTACCTCTGGCGCCGCTAG |
| 111 | GBA1 GS_GCU | ATGGAGTTCAGCAGCCCCTCAAGGGAGGAGTGCCCGAAGCCGCTCAGCCGCGTCAGCATCATGGCCGGGAGCCTGACGGGCC |
| | ORF | TGCTGCTGCTGCAGGCTGTCAGCTGGGCGAGCGGTGCCCGCCCGTGCATCCCCAAGAGCTTCGGCTACAGCAGCGTGGTGTGC |
| | | GTGTGCAACGCGACCTACTGCGACAGCTTCGACCCACCCACCTTCCCGGCACTGGGCACGTTCAGCCGCTACGAGTCGACTCG |
| | | CAGCGGCCGCCGCATGGAGCTCAGCATGGGCCCGATCCAGGCCAACCACACGGGCACGGGCCTGCTGCTGACGCTGCAGCCA |
| | | GAGCAGAAGTTCCAGAAGGTGAAGGGGTTCGGTGGTGCCATGACGGACGCTGCTGCACTGAACATCCTGGCACTCAGCCCAC |
| | | CCGCGCAGAATCTCCTGCTGAAGAGCTACTTCAGCGAGGAGGGCATCGGCTACAACATCATCCGCGTTCCCATGGCGAGCTGC |
| | | GACTTCAGCATCCGCACCTACACCTACGCGGACACTCCTGACGACTTCCAGCTGCACAACTTCAGCCTTCCAGAGGAGGACAC |
| | | GAAGCTGAAGATCCCCCTGATCCACCGCGCACTGCAGCTGGCGCAGCGCCCGGTCAGCCTGCTGGCGAGCCCGTGGACGAGC |
| | | CCCACCTGGCTGAAGACGAATGGTGCTGTGAATGGGAAGGGGAGCCTGAAGGGGCAGCCCGGCGACATCTACCACCAGACGT |
| | | GGGCCCGCTACTTCGTGAAGTTCCTGGACGCGTACGCGGAGCACAAGCTGCAGTTCTGGGCTGTGACGGCGGAGAACGAGCC |
| | | CTCAGCCGGGCTGCTCAGCGGCTACCCGTTCCAGTGCCTGGGCTTCACTCCAGAGCACCAGCGAGACTTCATCGCCCGAGACC |
| | | TGGGCCCCACCCTGGCGAACTCGACTCACCACAACGTGCGCCTGCTGATGCTGGACGACCAGCGCCTGCTGCTTCCACACTGG |
| | | GCGAAGGTGGTGCTGACGGACCCAGAAGCTGCCAAGTACGTGCACGGCATCGCTGTGCACTGGTACCTGGACTTCCTGGCGCC |
| | | GGCGAAGGCGACGCTGGGTGAGACGCACCGCCTGTTCCCGAACACGATGCTGTTCGCGAGCGAAGCCTGCGTTGGCAGCAAG |
| | | TTCTGGGAGCAGAGCGTGCGCCTGGGCAGCTGGGACCGCGGCATGCAGTACAGCCACAGCATCATCACGAATCTCCTGTACCA |
| | | CGTGGTTGGCTGGACGGACTGGAATCTCGCACTGAACCCAGAGGGTGGCCCGAACTGGGTGCGCAACTTCGTGGACAGCCCG |
| | | ATCATCGTGGACATCACGAAGGACACGTTCTACAAGCAGCCCATGTTCTACCACCTGGGCCACTTCAGCAAGTTCATCCCAGA |
| | | GGGCAGCCAGCGCGTTGGCCTGGTGGCGAGCCAGAAGAACGACCTGGACGCTGTGGCACTGATGCACCCTGACGGCAGCGCT |
| | | GTGGTGGTGGTGCTGAACCGCAGCAGCAAGGACGTTCCCCTGACGATCAAGGACCCGGCTGTTGGCTTCCTGGAGACGATCAG |
| | | CCCGGGCTACAGCATCCACACCTACCTGTGGCGCCGCTAG |
| 112 | GLA amino acid | MQLRNPELHLGCALALRFLALVSWDIPGARALDNGLARTPTMGWLHWERFMCNLDCQEEPDSCISEKLFMEMAELMVSEGWKDA |
| | sequence | GYEYLCIDDCWMAPQRDSEGRLQADPQRFPHGIRQLANYVHSKGLKLGIYADVGNKTCAGFPGSFGYYDIDAQTFADWGVDLLKF |
| | | DGCYCDSLENLADGYKHMSLALNRTGRSIVYSCEWPLYMWPFQKPNYTEIRQYCNHWRNFADIDDSWKSIKSILDWTSFNQERIVD |
| | | VAGPGGWNDPDMLVIGNFGLSWNQQVTQMALWAIMAAPLFMSNDLRHISPQAKALLQDKDVIAINQDPLGKQGYQLRQGDNFEV |
| | | WERPLSGLAWAVAMINRQEIGGPRSYTIAVASLGKGVACNPACFITQLLPVKRKLGFYEWTSRLRSHINPTGTVLLQLENTMQMSL |
| | | KDL |
| 113 | GLA WT ORF | AUGCAAUUACGUAAUCCUGAAUUACAUUUAGGUUGUGCUUUAGCUUUACGUUUUUUAGCUUUAGUUUCUUGGGAUAUUC |
| | | CUGGUGCUCGUGCUUUAGAUAAUGGUUUAGCUCGUACUCCUACUAUGGGUUGGUUACAUUGGGAACGUUUUAUGUGUAA |
| | | UUUAGAUUGUCAAGAAGAACCUGAUUCUUGUAUUUCUGAAAAAUUAUUUAUGGAAAUGGCUGAAUUAAUGGUUUCUGAA |
| | | GGUUGGAAAGAUGCUGGUUAUGAAUAUUUAUGUAUUGAUGAUUGUUGGAUGGCUCCUCAACGUGAUUCUGAAGGUCGUU |
| | | UACAAGCUGAUCCUCAACGUUUUCCUCAUGGUAUUCGUCAAUUAGCUAAUUAUGUUCAUUCUAAAGGUUUAAAAUUAGG |
| | | UAUUUAUGCUGAUGUUGGUAAUAAAACUUGUGCUGGUUUUCCUGGUUCUUUUGGUUAUUAUGAUAUUGAUGCUCAAACU |
| | | UUUGCUGAUUGGGGUGUUGAUUUAUUAAAAUUUGAUGGUUGUUAUUGUGAUUCUUUAGAAAAUUUAGCUGAUGGUUAUA |
| | | AACAUAUGUCUUUAGCUUUAAAUCGUACUGGUCGUUCUAUUGUUUAUUCUUGUGAAUGGCCUUUAUAUAUGUGGCCUUU |
| | | UCAAAAACCUAAUUAUACUGAAAUUCGUCAAUAUUGUAAUCAUUGGCGUAAUUUUGCUGAUAUUGAUGAUUCUUGGAAA |
| | | UCUAUUAAAUCUAUUUUAGAUUGGACUUCUUUUAAUCAAGAACGUAUUGUUGAUGUUGCUGGUCCUGGUGGUUGGAAUG |
| | | AUCCUGAUAUGUUAGUUAUUGGUAAUUUUGGUUUAUCUUGGAAUCAACAAGUUACUCAAAUGGCUUUAUGGGCUAUUAU |
| | | GGCUGCUCCUUUAUUUAUGUCUAAUGAUUUACGUCAUAUUUCUCCUCAAGCUAAAGCUUUAUUACAAGAUAAAGAUGUU |
| | | AUUGCUAUUAAUCAAGAUCCUUUAGGUAAACAAGGUUAUCAAUUACGUCAAGGUGAUAAUUUUGAAGUUUGGGAACGUC |
| | | CUUUAUCUGGUUUAGCUUGGGCUGUUGCUAUGAUUAAUCGUCAAGAAAUUGGUGGUCCUCGUUCUUAUACUAUUGCUGU |
| | | UGCUUCUUUAGGUAAAGGUGUUGCUUGUAAUCCUGCUUGUUUUAUUACUCAAUUAUUACCUGUUAAACGUAAAUUAGGU |
| | | UUUUAUGAAUGGACUUCUCGUUUACGUUCUCAUAUUAAUCCUACUGGUACUGUUUUAUUACAAUUAGAAAAUACUAUGC |
| | | AAAUGUCUUUAAAAGAUUUAUAG |
| 114 | GLA BP_GCU | ATGCAGCTCCGCAACCCCGAGCTCCACCTCGGCTGCGCGCTCGCGCTCCGCTTCCTCGCGCTCGTCTCGTGGGACATCCCCGGC |
| | ORF | GCGCGCGCGCTCGACAACGGCCTCGCGCGCACGCCCACGATGGGCTGGCTCCACTGGGAGCGCTTCATGTGCAACCTCGACTG |
| | | CCAGGAGGAGCCCGACTCGTGCATCTCGGAGAAGCTCTTCATGGAGATGGCGGAGCTCATGGTCTCGGAGGGCTGGAAGGAC |
| | | GCGGGCTACGAGTACCTCTGCATCGACGACTGCTGGATGGCGCCCCAGCGCGACTCGGAGGGCCGCCTCCAGGCGGACCCCC |
| | | AGCGCTTCCCCCACGGCATCCGCCAGCTCGCGAACTACGTCCACTCGAAGGGCCTCAAGCTCGGCATCTACGCGGACGTCGGC |
| | | AACAAGACGTGCGCGGGCTTCCCCGGCTCGTTCGGCTACTACGACATCGACGCGCAGACGTTCGCGGACTGGGGCGTCGACCT |
| | | CCTCAAGTTCGACGGCTGCTACTGCGACTCGCTCGAGAACCTCGCGGACGGCTACAAGCACATGTCGCTCGCGCTCAACCGCA |
| | | CGGGCCGCTCGATCGTCTACTCGTGCGAGTGGCCCCTCTACATGTGGCCCTTCCAGAAGCCCAACTACACGGAGATCCGCCAG |
| | | TACTGCAACCACTGGCGCAACTTCGCGGACATCGACGACTCGTGGAAGTCGATCAAGTCGATCCTCGACTGGACGTCGTTCAA |
| | | CCAGGAGCGCATCGTCGACGTCGCGGGCCCCGGCGGCTGGAACGACCCCGACATGCTCGTCATCGGCAACTTCGGCCTCTCGT |
| | | GGAACCAGCAGGTCACGCAGATGGCGCTCTGGGCGATCATGGCGGCGCCCCTCTTCATGTCGAACGACCTCCGCCACATCTCG |
| | | CCCCAGGCGAAGGCGCTCCTCCAGGACAAGGACGTCATCGCGATCAACCAGGACCCCCTCGGCAAGCAGGGCTACCAGCTCC |
| | | GCCAGGGCGACAACTTCGAGGTCTGGGAGCGCCCCCTCTCGGGCCTCGCGTGGGCGGTCGCGATGATCAACCGCCAGGAGAT |
| | | CGGCGGCCCCCGCTCGTACACGATCGCGGTCGCGTCGCTCGGCAAGGGCGTCGCGTGCAACCCCGCGTGCTTCATCACGCAGC |
| | | TCCTCCCCGTCAAGCGCAAGCTCGGCTTCTACGAGTGGACGTCGCGCCTCCGCTCGCACATCAACCCCACGGGCACGGTCCTC |
| | | CTCCAGCTCGAGAACACGATGCAGATGTCGCTCAAGGACCTCTAG |
| 115 | GLA | ATGCAGCTGCGGAACCCAGAGCTGCACCTCGGCTGCGCACTGGCACTGCGGTTCCTGGCACTGGTCAGCTGGGACATCCCCGG |
| | GP_BP_BS_GCU | TGCCCGCGCACTGGACAATGGGCTCGCCCGCACTCCCACCATGGGCTGGCTCCACTGGGAGCGCTTCATGTGCAATCTCGACT |
| | ORF | GCCAGGAGGAGCCTGACTCGTGCATCTCGGAGAAGCTCTTCATGGAGATGGCGGAGCTGATGGTCAGCGAGGGCTGGAAGGA |
| | | CGCCGGGTACGAGTACCTCTGCATCGACGACTGCTGGATGGCGCCCCAGCGAGACTCGGAGGGCCGCCTCCAGGCCGACCCC |
| | | CAGCGCTTCCCCCACGGCATCCGCCAGCTCGCGAACTACGTCCACTCGAAGGGGCTCAAGCTCGGCATCTACGCGGACGTTGG |
| | | CAACAAGACGTGCGCCGGGTTCCCTGGCTCGTTCGGCTACTACGACATCGACGCGCAGACGTTCGCGGACTGGGGCGTCGACC |
| | | TCCTCAAGTTCGACGGCTGCTACTGCGACTCGCTCGAGAATCTCGCGGACGGCTACAAGCACATGTCGCTCGCACTGAACCGC |
| | | ACGGGCCGAAGCATCGTCTACTCGTGCGAGTGGCCCCTCTACATGTGGCCCTTCCAGAAGCCCAACTACACAGAGATCCGCCA |
| | | GTACTGCAACCACTGGCGCAACTTCGCGGACATCGACGACTCGTGGAAGAGCATCAAGAGCATCCTCGACTGGACGTCGTTCA |
| | | ACCAGGAGCGCATCGTCGACGTCGCCGGGCCTGGTGGCTGGAACGACCCTGACATGCTCGTCATCGGCAACTTCGGCCTCAGC |
| | | TGGAACCAGCAGGTGACTCAAATGGCACTGTGGGCGATCATGGCTGCCCCCCTCTTCATGTCGAACGACCTGCGGCACATCTC |
| | | GCCCCAGGCCAAGGCACTGCTCCAGGACAAGGACGTCATCGCGATCAACCAGGACCCCCTCGGCAAGCAGGGCTACCAGCTG |
| | | CGGCAGGGCGACAACTTCGAAGTCTGGGAGCGCCCCCTCAGCGGCCTCGCGTGGGCTGTGGCGATGATAAACCGCCAGGAGA |
| | | TCGGTGGCCCCCGATCGTACACGATCGCTGTGGCGTCGCTCGGCAAGGGGGTCGCGTGCAACCCTGCGTGCTTCATCACTCAA |
| | | CTCCTTCCAGTCAAGCGCAAGCTCGGCTTCTACGAGTGGACGTCGCGCCTGCGGTCGCACATCAACCCCACCGGCACGGTCCT |
| | | CCTCCAGCTCGAGAACACGATGCAGATGTCGCTCAAGGACCTCTAG |
| 116 | GLA | ATGCAGCTGCGGAACCCAGAGCTGCACCTCGGCTGCGCACTGGCACTGCGGTTCCTGGCACTGGTCAGCTGGGACATCCCCGG |
| | GS_BS_GCU | TGCCCGCGCACTGGACAATGGGCTCGCCCGCACTCCCACCATGGGCTGGCTCCACTGGGAGCGCTTCATGTGCAATCTCGACT |
| | ORF | GCCAGGAGGAGCCTGACAGCTGCATCAGCGAGAAGCTCTTCATGGAGATGGCGGAGCTGATGGTCAGCGAGGGCTGGAAGGA |
| | | CGCCGGGTACGAGTACCTCTGCATCGACGACTGCTGGATGGCGCCCCAGCGAGACAGCGAGGGCCGCCTCCAGGCCGACCCC |
| | | CAGCGCTTCCCCCACGGCATCCGCCAGCTCGCGAACTACGTCCACAGCAAGGGGCTCAAGCTCGGCATCTACGCGGACGTTGG |
| | | CAACAAGACGTGCGCCGGGTTCCCTGGCAGCTTCGGCTACTACGACATCGACGCGCAGACGTTCGCGGACTGGGGCGTCGACC |
| | | TCCTCAAGTTCGACGGCTGCTACTGCGACAGCCTCGAGAATCTCGCGGACGGCTACAAGCACATGAGCCTCGCACTGAACCGC |
| | | ACGGGCAGGAGCATCGTCTACAGCTGCGAGTGGCCCCTCTACATGTGGCCCTTCCAGAAGCCCAACTACACAGAGATCCGCCA |
| | | GTACTGCAACCACTGGCGCAACTTCGCGGACATCGACGACAGCTGGAAGAGCATCAAGAGCATCCTCGACTGGACGAGCTTC |
| | | AACCAGGAGCGCATCGTCGACGTCGCCGGGCCTGGTGGCTGGAACGACCCTGACATGCTCGTCATCGGCAACTTCGGCCTCAG |
| | | CTGGAACCAGCAGGTGACTCAAATGGCACTGTGGGCGATCATGGCTGCCCCCCTCTTCATGAGCAACGACCTGCGGCACATCA |
| | | GCCCCCAGGCCAAGGCACTGCTCCAGGACAAGGACGTCATCGCGATCAACCAGGACCCCCTCGGCAAGCAGGGCTACCAGCT |
| | | GCGGCAGGGCGACAACTTCGAAGTCTGGGAGCGCCCCCTCAGCGGCCTCGCGTGGGCTGTGGCGATGATAAACCGCCAGGAG |
| | | ATCGGTGGCCCCAGGAGCTACACGATCGCTGTGGCGAGCCTCGGCAAGGGGGTCGCGTGCAACCCTGCGTGCTTCATCACTCA |
| | | ACTCCTTCCAGTCAAGCGCAAGCTCGGCTTCTACGAGTGGACGAGCCGCCTGCGGAGCCACATCAACCCCACCGGCACGGTCC |
| | | TCCTCCAGCTCGAGAACACGATGCAGATGAGCCTCAAGGACCTCTAG |
| 117 | GLA GS_GCU | ATGCAGCTGCGGAACCCAGAGCTGCACCTGGGCTGCGCACTGGCACTGCGGTTCCTGGCACTGGTCAGCTGGGACATCCCCGG |
| | ORF | TGCCCGCGCACTGGACAATGGGCTGGCCCGCACTCCCACCATGGGCTGGCTGCACTGGGAGCGCTTCATGTGCAATCTCGACT |
| | | GCCAGGAGGAGCCTGACAGCTGCATCAGCGAGAAGCTGTTCATGGAGATGGCGGAGCTGATGGTCAGCGAGGGCTGGAAGG |
| | | ACGCCGGGTACGAGTACCTGTGCATCGACGACTGCTGGATGGCGCCGCAGCGAGACAGCGAGGGCCGCCTGCAGGCCGACCC |
| | | GCAGCGCTTCCCGCACGGCATCCGCCAGCTGGCGAACTACGTGCACAGCAAGGGGCTGAAGCTGGGCATCTACGCGGACGTT |
| | | GGCAACAAGACGTGCGCCGGGTTCCCGGGCAGCTTCGGCTACTACGACATCGACGCGCAGACGTTCGCGGACTGGGGCGTGG |
| | | ACCTGCTGAAGTTCGACGGCTGCTACTGCGACAGCCTGGAGAATCTCGCGGACGGCTACAAGCACATGAGCCTGGCACTGAA |
| | | CCGCACGGGCCGCAGCATCGTGTACAGCTGCGAGTGGCCGCTGTACATGTGGCCGTTCCAGAAGCCGAACTACACAGAGATC |
| | | CGCCAGTACTGCAACCACTGGCGCAACTTCGCGGACATCGACGACAGCTGGAAGAGCATCAAGAGCATCCTGGACTGGACGA |
| | | GCTTCAACCAGGAGCGCATCGTGGACGTGGCCGGGCCGGGTGGCTGGAACGACCCTGACATGCTGGTGATCGGCAACTTCGG |
| | | CCTCAGCTGGAACCAGCAGGTGACTCAAATGGCACTGTGGGCGATCATGGCTGCCCCGCTGTTCATGAGCAACGACCTGCGGC |
| | | ACATCAGCCCGCAGGCCAAGGCACTGCTGCAGGACAAGGACGTGATCGCGATCAACCAGGACCCGCTGGGCAAGCAGGGCTA |
| | | CCAGCTGCGGCAGGGCGACAACTTCGAAGTCTGGGAGCGCCCGCTCAGCGGCCTGGCGTGGGCTGTGGCGATGATAAACCGC |
| | | CAGGAGATCGGTGGCCCGCGCAGCTACACGATCGCTGTGGCGAGCCTGGGCAAGGGGGTGGCGTGCAACCCGGCGTGCTTCA |
| | | TCACTCAACTGCTTCCAGTGAAGCGCAAGCTGGGCTTCTACGAGTGGACGAGCCGCCTGCGGAGCCACATCAACCCCACCGGC |
| | | ACGGTGCTGCTGCAGCTGGAGAACACGATGCAGATGAGCCTGAAGGACCTGTAG |
| 118 | OTC amino acid | MLFNLRILLNNAAFRNGHNFMVRNFRCGQPLQNKVQLKGRDLLTLKNFTGEEIKYMLWLSADLKFRIKQKGEYLPLLQGKSLGMIF |
| | sequence | EKRSTRTRLSTETGFALLGGHPCFLTTQDIHLGVNESLTDTARVLSSMADAVLARVYKQSDLDTLAKEASIPIINGLSDLYHPIQILAD |
| | | YLTLQEHYSSLKGLTLSWIGDGNNILHSIMMSAAKFGMHLQAATPKGYEPDASVTKLAEQYAKENGTKLLLTNDPLEAAHGGNVLI |
| | | TDTWISMGQEEEKKKRLQAFQGYQVTMKTAKVAASDWTFLHCLPRKPEEVDDEVFYSPRSLVFPEAENRKWTIMAVMVSLLTDY |
| | | SPQLQKPK |
| 119 | OTC WT ORF | AUGUUAUUUAAUUUACGUAUUUUAUUAAAUAAUGCUGCUUUUCGUAAUGGUCAUAAUUUUAUGGUUCGUAAUUUUCGUU |
| | | GUGGUCAACCUUUACAAAAUAAAGUUCAAUUAAAAGGUCGUGAUUUAUUAACUUUAAAAAAUUUUACUGGUGAAGAAAU |
| | | UAAAUAUAUGUUAUGGUUAUCUGCUGAUUUAAAAUUUCGUAUUAAACAAAAAGGUGAAUAUUUACCUUUAUUACAAGGU |
| | | AAAUCUUUAGGUAUGAUUUUUGAAAAACGUUCUACUCGUACUCGUUUAUCUACUGAAACUGGUUUUGCUUUAUUAGGUG |
| | | GUCAUCCUUGUUUUUUAACUACUCAAGAUAUUCAUUUAGGUGUUAAUGAAUCUUUAACUGAUACUGCUCGUGUUUUAUC |
| | | UUCUAUGGCUGAUGCUGUUUUAGCUCGUGUUUAUAAACAAUCUGAUUUAGAUACUUUAGCUAAAGAAGCUUCUAUUCCU |
| | | AUUAUUAAUGGUUUAUCUGAUUUAUAUCAUCCUAUUCAAAUUUUAGCUGAUUAUUUAACUUUACAAGAACAUUAUUCUU |
| | | CUUUAAAAGGUUUAACUUUAUCUUGGAUUGGUGAUGGUAAUAAUAUUUUACAUUCUAUUAUGAUGUCUGCUGCUAAAUU |
| | | UGGUAUGCAUUUACAAGCUGCUACUCCUAAAGGUUAUGAACCUGAUGCUUCUGUUACUAAAUUAGCUGAACAAUAUGCUA |
| | | AAGAAAAUGGUACUAAAUUAUUAUUAACUAAUGAUCCUUUAGAAGCUGCUCAUGGUGGUAAUGUUUUAAUUACUGAUAC |
| | | UUGGAUUUCUAUGGGUCAAGAAGAAGAAAAAAAAAAACGUUUACAAGCUUUUCAAGGUUAUCAAGUUACUAUGAAAACU |
| | | GCUAAAGUUGCUGCUUCUGAUUGGACUUUUUUACAUUGUUUACCUCGUAAACCUGAAGAAGUUGAUGAUGAAGUUUUUU |
| | | AUUCUCCUCGUUCUUUAGUUUUUCCUGAAGCUGAAAAUCGUAAAUGGACUAUUAUGGCUGUUAUGGUUUCUUUAUUAAC |
| | | UGAUUAUUCUCCUCAAUUACAAAAACCUAAAUAG |
| 120 | OTC BP_GCU | ATGCTCTTCAACCTCCGCATCCTCCTCAACAACGCGGCGTTCCGCAACGGCCACAACTTCATGGTCCGCAACTTCCGCTGCGGC |
| | ORF | CAGCCCCTCCAGAACAAGGTCCAGCTCAAGGGCCGCGACCTCCTCACGCTCAAGAACTTCACGGGCGAGGAGATCAAGTACA |
| | | TGCTCTGGCTCTCGGCGGACCTCAAGTTCCGCATCAAGCAGAAGGGCGAGTACCTCCCCCTCCTCCAGGGCAAGTCGCTCGGC |
| | | ATGATCTTCGAGAAGCGCTCGACGCGCACGCGCCTCTCGACGGAGACGGGCTTCGCGCTCCTCGGCGGCCACCCCTGCTTCCT |
| | | CACGACGCAGGACATCCACCTCGGCGTCAACGAGTCGCTCACGGACACGGCGCGCGTCCTCTCGTCGATGGCGGACGCGGTCC |
| | | TCGCGCGCGTCTACAAGCAGTCGGACCTCGACACGCTCGCGAAGGAGGCGTCGATCCCCATCATCAACGGCCTCTCGGACCTC |
| | | TACCACCCCATCCAGATCCTCGCGGACTACCTCACGCTCCAGGAGCACTACTCGTCGCTCAAGGGCCTCACGCTCTCGTGGATC |
| | | GGCGACGGCAACAACATCCTCCACTCGATCATGATGTCGGCGGCGAAGTTCGGCATGCACCTCCAGGCGGCGACGCCCAAGG |
| | | GCTACGAGCCCGACGCGTCGGTCACGAAGCTCGCGGAGCAGTACGCGAAGGAGAACGGCACGAAGCTCCTCCTCACGAACGA |
| | | CCCCCTCGAGGCGGCGCACGGCGGCAACGTCCTCATCACGGACACGTGGATCTCGATGGGCCAGGAGGAGGAGAAGAAGAA |
| | | GCGCCTCCAGGCGTTCCAGGGCTACCAGGTCACGATGAAGACGGCGAAGGTCGCGGCGTCGGACTGGACGTTCCTCCACTGCC |
| | | TCCCCCGCAAGCCCGAGGAGGTCGACGACGAGGTCTTCTACTCGCCCCGCTCGCTCGTCTTCCCCGAGGCGGAGAACCGCAAG |
| | | TGGACGATCATGGCGGTCATGGTCTCGCTCCTCACGGACTACTCGCCCCAGCTCCAGAAGCCCAAGTAG |
| 121 | OTC | ATGCTCTTCAATCTGCGGATCCTCCTCAACAACGCTGCCTTCCGCAATGGGCACAACTTCATGGTCCGCAACTTCCGCTGTGGG |
| | GP_BP_BS_GCU | CAGCCCCTCCAGAACAAGGTCCAGCTCAAGGGGCGAGACCTCCTCACGCTCAAGAACTTCACGGGTGAGGAGATCAAGTACA |
| | ORF | TGCTCTGGCTCAGCGCGGACCTCAAGTTCCGCATCAAGCAGAAGGGTGAGTACCTTCCACTCCTCCAGGGCAAGTCGCTCGGC |
| | | ATGATATTCGAGAAGCGATCGACTCGCACGCGCCTCTCGACAGAGACGGGCTTCGCACTGCTCGGTGGCCACCCCTGCTTCCT |
| | | GACGACTCAAGACATCCACCTCGGCGTCAACGAGTCGCTCACGGACACGGCCCGCGTCCTCAGCTCGATGGCGGACGCTGTGC |
| | | TCGCCCGCGTCTACAAGCAGAGCGACCTCGACACGCTCGCGAAGGAAGCCAGCATCCCCATCATCAATGGGCTCAGCGACCTC |
| | | TACCACCCCATCCAGATCCTCGCGGACTACCTCACGCTCCAGGAGCACTACTCGTCGCTCAAGGGGCTCACGCTCAGCTGGAT |
| | | CGGCGACGGCAACAACATCCTCCACAGCATCATGATGTCGGCTGCCAAGTTCGGCATGCACCTCCAGGCTGCCACTCCCAAGG |
| | | GGTACGAGCCTGACGCGTCGGTCACGAAGCTCGCGGAGCAGTACGCGAAGGAGAATGGGACGAAGCTCCTCCTCACGAACGA |
| | | CCCCCTCGAAGCTGCCCACGGTGGCAACGTCCTCATCACGGACACGTGGATCTCGATGGGCCAGGAGGAGGAGAAGAAGAAG |
| | | CGCCTCCAGGCCTTCCAGGGCTACCAGGTGACGATGAAGACGGCGAAGGTCGCTGCCTCGGACTGGACGTTCCTGCACTGCCT |
| | | TCCACGCAAGCCAGAGGAAGTCGACGACGAAGTCTTCTACTCGCCCCGATCGCTCGTCTTCCCAGAAGCCGAGAACCGCAAGT |
| | | GGACGATCATGGCTGTGATGGTCAGCCTCCTCACGGACTACTCGCCCCAGCTCCAGAAGCCCAAGTAG |
| 122 | OTC | ATGCTCTTCAATCTGCGGATCCTCCTCAACAACGCTGCCTTCCGCAATGGGCACAACTTCATGGTCCGCAACTTCCGCTGTGGG |
| | GS_BS_GCU | CAGCCCCTCCAGAACAAGGTCCAGCTCAAGGGGCGAGACCTCCTCACGCTCAAGAACTTCACGGGTGAGGAGATCAAGTACA |
| | ORF | TGCTCTGGCTCAGCGCGGACCTCAAGTTCCGCATCAAGCAGAAGGGTGAGTACCTTCCACTCCTCCAGGGCAAGAGCCTCGGC |
| | | ATGATATTCGAGAAGAGGTCGACTCGCACGCGCCTCTCGACAGAGACGGGCTTCGCACTGCTCGGTGGCCACCCCTGCTTCCT |
| | | GACGACTCAAGACATCCACCTCGGCGTCAACGAGAGCCTCACGGACACGGCCCGCGTCCTCAGCAGCATGGCGGACGCTGTG |
| | | CTCGCCCGCGTCTACAAGCAGAGCGACCTCGACACGCTCGCGAAGGAAGCCAGCATCCCCATCATCAATGGGCTCAGCGACCT |
| | | CTACCACCCCATCCAGATCCTCGCGGACTACCTCACGCTCCAGGAGCACTACAGCAGCCTCAAGGGGCTCACGCTCAGCTGGA |
| | | TCGGCGACGGCAACAACATCCTCCACAGCATCATGATGAGCGCTGCCAAGTTCGGCATGCACCTCCAGGCTGCCACTCCCAAG |
| | | GGGTACGAGCCTGACGCGAGCGTCACGAAGCTCGCGGAGCAGTACGCGAAGGAGAATGGGACGAAGCTCCTCCTCACGAACG |
| | | ACCCCCTCGAAGCTGCCCACGGTGGCAACGTCCTCATCACGGACACGTGGATCAGCATGGGCCAGGAGGAGGAGAAGAAGAA |
| | | GCGCCTCCAGGCCTTCCAGGGCTACCAGGTGACGATGAAGACGGCGAAGGTCGCTGCCAGCGACTGGACGTTCCTGCACTGCC |
| | | TTCCACGCAAGCCAGAGGAAGTCGACGACGAAGTCTTCTACAGCCCCAGGAGCCTCGTCTTCCCAGAAGCCGAGAACCGCAA |
| | | GTGGACGATCATGGCTGTGATGGTCAGCCTCCTCACGGACTACAGCCCCCAGCTCCAGAAGCCCAAGTAG |
| 123 | OTC GS_GCU | ATGCTGTTCAATCTGCGGATCCTGCTGAACAACGCTGCCTTCCGCAATGGGCACAACTTCATGGTGCGCAACTTCCGCTGTGGG |
| | ORF | CAGCCCCTGCAGAACAAGGTGCAGCTGAAGGGGCGAGACCTGCTGACGCTGAAGAACTTCACGGGTGAGGAGATCAAGTACA |
| | | TGCTGTGGCTCAGCGCGGACCTGAAGTTCCGCATCAAGCAGAAGGGTGAGTACCTTCCACTGCTGCAGGGCAAGAGCCTGGG |
| | | CATGATATTCGAGAAGCGCTCGACTCGCACGCGCCTCTCGACAGAGACGGGCTTCGCACTGCTGGGTGGCCACCCGTGCTTCC |
| | | TGACGACTCAAGACATCCACCTGGGCGTGAACGAGAGCCTGACGGACACGGCCCGCGTGCTCAGCAGCATGGCGGACGCTGT |
| | | GCTGGCCCGCGTGTACAAGCAGAGCGACCTGGACACGCTGGCGAAGGAAGCCAGCATCCCCATCATCAATGGGCTCAGCGAC |
| | | CTGTACCACCCGATCCAGATCCTGGCGGACTACCTGACGCTGCAGGAGCACTACAGCAGCCTGAAGGGGCTGACGCTCAGCTG |
| | | GATCGGCGACGGCAACAACATCCTGCACAGCATCATGATGAGCGCTGCCAAGTTCGGCATGCACCTGCAGGCTGCCACTCCCA |
| | | AGGGGTACGAGCCTGACGCGAGCGTGACGAAGCTGGCGGAGCAGTACGCGAAGGAGAATGGGACGAAGCTGCTGCTGACGA |
| | | ACGACCCGCTGGAAGCTGCCCACGGTGGCAACGTGCTGATCACGGACACGTGGATCAGCATGGGCCAGGAGGAGGAGAAGA |
| | | AGAAGCGCCTGCAGGCCTTCCAGGGCTACCAGGTGACGATGAAGACGGCGAAGGTGGCTGCCAGCGACTGGACGTTCCTGCA |
| | | CTGCCTTCCACGCAAGCCAGAGGAAGTCGACGACGAAGTCTTCTACAGCCCGCGCAGCCTGGTGTTCCCAGAAGCCGAGAAC |
| | | CGCAAGTGGACGATCATGGCTGTGATGGTCAGCCTGCTGACGGACTACAGCCCGCAGCTGCAGAAGCCGAAGTAG |
| 124 | PAH amino acid | MSTAVLENPGLGRKLSDFGQETSYIEDNCNQNGAISLIFSLKEEVGALAKVLRLFEENDVNLTHIESRPSRLKKDEYEFFTHLDKRSL |
| | sequence | PALTNIIKILRHDIGATVHELSRDKKKDTVPWFPRTIQELDRFANQILSYGAELDADHPGFKDPVYRARRKQFADIAYNYRHGQPIPR |
| | | VEYMEEEKKTWGTVFKTLKSLYKTHACYEYNHIFPLLEKYCGFHEDNIPQLEDVSQFLQTCTGFRLRPVAGLLSSRDFLGGLAFRVF |
| | | HCTQYIRHGSKPMYTPEPDICHELLGHVPLFSDRSFAQFSQEIGLASLGAPDEYIEKLATIYWFTVEFGLCKQGDSIKAYGAGLLSSFG |
| | | ELQYCLSEKPKLLPLELEKTAIQNYTVTEFQPLYYVAESFNDAKEKVRNFAAT1PRPFSVRYDPYTQRIEVLDNTQQLKILADSINSEI |
| | | GILCSALQKI |
| 125 | PAH WT ORF | AUGUCUACUGCUGUUUUAGAAAAUCCUGGUUUAGGUCGUAAAUUAUCUGAUUUUGGUCAAGAAACUUCUUAUAUUGAAG |
| | | AUAAUUGUAAUCAAAAUGGUGCUAUUUCUUUAAUUUUUUCUUUAAAAGAAGAAGUUGGUGCUUUAGCUAAAGUUUUACG |
| | | UUUAUUUGAAGAAAAUGAUGUUAAUUUAACUCAUAUUGAAUCUCGUCCUUCUCGUUUAAAAAAAGAUGAAUAUGAAUUU |
| | | UUUACUCAUUUAGAUAAACGUUCUUUACCUGCUUUAACUAAUAUUAUUAAAAUUUUACGUCAUGAUAUUGGUGCUACUG |
| | | UUCAUGAAUUAUCUCGUGAUAAAAAAAAAGAUACUGUUCCUUGGUUUCCUCGUACUAUUCAAGAAUUAGAUCGUUUUGC |
| | | UAAUCAAAUUUUAUCUUAUGGUGCUGAAUUAGAUGCUGAUCAUCCUGGUUUUAAAGAUCCUGUUUAUCGUGCUCGUCGU |
| | | AAACAAUUUGCUGAUAUUGCUUAUAAUUAUCGUCAUGGUCAACCUAUUCCUCGUGUUGAAUAUAUGGAAGAAGAAAAAA |
| | | AAACUUGGGGUACUGUUUUUAAAACUUUAAAAUCUUUAUAUAAAACUCAUGCUUGUUAUGAAUAUAAUCAUAUUUUUCC |
| | | UUUAUUAGAAAAAUAUUGUGGUUUUCAUGAAGAUAAUAUUCCUCAAUUAGAAGAUGUUUCUCAAUUUUUACAAACUUGU |
| | | ACUGGUUUUCGUUUACGUCCUGUUGCUGGUUUAUUAUCUUCUCGUGAUUUUUUAGGUGGUUUAGCUUUUCGUGUUUUUC |
| | | AUUGUACUCAAUAUAUUCGUCAUGGUUCUAAACCUAUGUAUACUCCUGAACCUGAUAUUUGUCAUGAAUUAUUAGGUCA |
| | | UGUUCCUUUAUUUUCUGAUCGUUCUUUUGCUCAAUUUUCUCAAGAAAUUGGUUUAGCUUCUUUAGGUGCUCCUGAUGAA |
| | | UAUAUUGAAAAAUUAGCUACUAUUUAUUGGUUUACUGUUGAAUUUGGUUUAUGUAAACAAGGUGAUUCUAUUAAAGCUU |
| | | AUGGUGCUGGUUUAUUAUCUUCUUUUGGUGAAUUACAAUAUUGUUUAUCUGAAAAACCUAAAUUAUUACCUUUAGAAUU |
| | | AGAAAAAACUGCUAUUCAAAAUUAUACUGUUACUGAAUUUCAACCUUUAUAUUAUGUUGCUGAAUCUUUUAAUGAUGCU |
| | | AAAGAAAAAGUUCGUAAUUUUGCUGCUACUAUUCCUCGUCCUUUUUCUGUUCGUUAUGAUCCUUAUACUCAACGUAUUGA |
| | | AGUUUUAGAUAAUACUCAACAAUUAAAAAUUUUAGCUGAUUCUAUUAAUUCUGAAAUUGGUAUUUUAUGUUCUGCUUUA |
| | | CAAAAAAUUUAG |
| 126 | PAH BP_GCU | ATGTCGACGGCGGTCCTCGAGAACCCCGGCCTCGGCCGCAAGCTCTCGGACTTCGGCCAGGAGACGTCGTACATCGAGGACA |
| | ORF | ACTGCAACCAGAACGGCGCGATCTCGCTCATCTTCTCGCTCAAGGAGGAGGTCGGCGCGCTCGCGAAGGTCCTCCGCCTCTTC |
| | | GAGGAGAACGACGTCAACCTCACGCACATCGAGTCGCGCCCCTCGCGCCTCAAGAAGGACGAGTACGAGTTCTTCACGCACC |
| | | TCGACAAGCGCTCGCTCCCCGCGCTCACGAACATCATCAAGATCCTCCGCCACGACATCGGCGCGACGGTCCACGAGCTCTCG |
| | | CGCGACAAGAAGAAGGACACGGTCCCCTGGTTCCCCCGCACGATCCAGGAGCTCGACCGCTTCGCGAACCAGATCCTCTCGTA |
| | | CGGCGCGGAGCTCGACGCGGACCACCCCGGCTTCAAGGACCCCGTCTACCGCGCGCGCCGCAAGCAGTTCGCGGACATCGCG |
| | | TACAACTACCGCCACGGCCAGCCCATCCCCCGCGTCGAGTACATGGAGGAGGAGAAGAAGACGTGGGGCACGGTCTTCAAGA |
| | | CGCTCAAGTCGCTCTACAAGACGCACGCGTGCTACGAGTACAACCACATCTTCCCCCTCCTCGAGAAGTACTGCGGCTTCCAC |
| | | GAGGACAACATCCCCCAGCTCGAGGACGTCTCGCAGTTCCTCCAGACGTGCACGGGCTTCCGCCTCCGCCCCGTCGCGGGCCT |
| | | CCTCTCGTCGCGCGACTTCCTCGGCGGCCTCGCGTTCCGCGTCTTCCACTGCACGCAGTACATCCGCCACGGCTCGAAGCCCAT |
| | | GTACACGCCCGAGCCCGACATCTGCCACGAGCTCCTCGGCCACGTCCCCCTCTTCTCGGACCGCTCGTTCGCGCAGTTCTCGCA |
| | | GGAGATCGGCCTCGCGTCGCTCGGCGCGCCCGACGAGTACATCGAGAAGCTCGCGACGATCTACTGGTTCACGGTCGAGTTCG |
| | | GCCTCTGCAAGCAGGGCGACTCGATCAAGGCGTACGGCGCGGGCCTCCTCTCGTCGTTCGGCGAGCTCCAGTACTGCCTCTCG |
| | | GAGAAGCCCAAGCTCCTCCCCCTCGAGCTCGAGAAGACGGCGATCCAGAACTACACGGTCACGGAGTTCCAGCCCCTCTACTA |
| | | CGTCGCGGAGTCGTTCAACGACGCGAAGGAGAAGGTCCGCAACTTCGCGGCGACGATCCCCCGCCCCTTCTCGGTCCGCTACG |
| | | ACCCCTACACGCAGCGCATCGAGGTCCTCGACAACACGCAGCAGCTCAAGATCCTCGCGGACTCGATCAACTCGGAGATCGG |
| | | CATCCTCTGCTCGGCGCTCCAGAAGATCTAG |
| 127 | PAH | ATGTCGACTGCTGTGCTCGAGAACCCTGGCCTCGGCCGCAAGCTCAGCGACTTCGGCCAGGAGACGTCGTACATCGAGGACAA |
| | GP_BP_BS_GCU | CTGCAACCAGAATGGTGCCATCTCGCTCATCTTCTCGCTCAAGGAGGAAGTTGGTGCACTGGCGAAGGTCCTGCGGCTCTTCG |
| | ORF | AGGAGAACGACGTCAATCTCACGCACATCGAGTCGCGCCCCTCACGCCTCAAGAAGGACGAGTACGAGTTCTTCACGCACCTC |
| | | GACAAGCGATCGCTTCCAGCACTGACGAACATCATCAAGATCCTGCGGCACGACATCGGTGCCACGGTCCACGAGCTCAGCC |
| | | GAGACAAGAAGAAGGACACGGTTCCCTGGTTCCCCCGCACGATCCAGGAGCTGGACCGCTTCGCGAACCAGATCCTCAGCTA |
| | | CGGTGCCGAGCTGGACGCGGACCACCCTGGCTTCAAGGACCCCGTCTACCGCGCCCGCCGCAAGCAGTTCGCGGACATCGCGT |
| | | ACAACTACCGCCACGGCCAGCCCATCCCCCGCGTCGAGTACATGGAGGAGGAGAAGAAGACGTGGGGCACGGTCTTCAAGAC |
| | | GCTCAAGTCGCTCTACAAGACGCACGCGTGCTACGAGTACAACCACATCTTCCCCCTCCTCGAGAAGTACTGTGGGTTCCACG |
| | | AGGACAACATCCCCCAGCTCGAGGACGTCAGCCAGTTCCTGCAGACGTGCACGGGCTTCCGCCTGCGGCCCGTCGCCGGGCTC |
| | | CTCAGCTCGCGAGACTTCCTGGGTGGCCTCGCGTTCCGCGTCTTCCACTGCACTCAATACATCCGCCACGGCTCGAAGCCCATG |
| | | TACACTCCAGAGCCTGACATCTGCCACGAGCTGCTCGGCCACGTTCCCCTCTTCTCGGACCGATCGTTCGCGCAGTTCTCGCAG |
| | | GAGATCGGCCTCGCGTCGCTCGGTGCCCCTGACGAGTACATCGAGAAGCTCGCGACGATCTACTGGTTCACGGTCGAGTTCGG |
| | | CCTCTGCAAGCAGGGCGACAGCATCAAGGCGTACGGTGCCGGGCTCCTCAGCTCGTTCGGTGAGCTGCAGTACTGCCTCAGCG |
| | | AGAAGCCCAAGCTCCTTCCACTCGAGCTGGAGAAGACGGCGATCCAGAACTACACGGTCACAGAGTTCCAGCCCCTCTACTAC |
| | | GTCGCGGAGTCGTTCAACGACGCGAAGGAGAAGGTCCGCAACTTCGCTGCCACGATCCCCCGCCCCTTCTCGGTCCGCTACGA |
| | | CCCCTACACTCAACGCATCGAAGTCCTCGACAACACTCAACAGCTCAAGATCCTCGCGGACAGCATCAACTCGGAGATCGGCA |
| | | TCCTCTGCTCGGCACTGCAGAAGATCTAG |
| 128 | PAH | ATGTCGACTGCTGTGCTCGAGAACCCTGGCCTCGGCCGCAAGCTCAGCGACTTCGGCCAGGAGACGAGCTACATCGAGGACA |
| | GS_BS_GCU | ACTGCAACCAGAATGGTGCCATCAGCCTCATCTTCAGCCTCAAGGAGGAAGTTGGTGCACTGGCGAAGGTCCTGCGGCTCTTC |
| | ORF | GAGGAGAACGACGTCAATCTCACGCACATCGAGAGCCGCCCCTCACGCCTCAAGAAGGACGAGTACGAGTTCTTCACGCACC |
| | | TCGACAAGAGGAGCCTTCCAGCACTGACGAACATCATCAAGATCCTGCGGCACGACATCGGTGCCACGGTCCACGAGCTCAG |
| | | CCGAGACAAGAAGAAGGACACGGTTCCCTGGTTCCCCCGCACGATCCAGGAGCTGGACCGCTTCGCGAACCAGATCCTCAGC |
| | | TACGGTGCCGAGCTGGACGCGGACCACCCTGGCTTCAAGGACCCCGTCTACCGCGCCCGCCGCAAGCAGTTCGCGGACATCGC |
| | | GTACAACTACCGCCACGGCCAGCCCATCCCCCGCGTCGAGTACATGGAGGAGGAGAAGAAGACGTGGGGCACGGTCTTCAAG |
| | | ACGCTCAAGAGCCTCTACAAGACGCACGCGTGCTACGAGTACAACCACATCTTCCCCCTCCTCGAGAAGTACTGTGGGTTCCA |
| | | CGAGGACAACATCCCCCAGCTCGAGGACGTCAGCCAGTTCCTGCAGACGTGCACGGGCTTCCGCCTGCGGCCCGTCGCCGGGC |
| | | TCCTCAGCAGCCGAGACTTCCTGGGTGGCCTCGCGTTCCGCGTCTTCCACTGCACTCAATACATCCGCCACGGCAGCAAGCCC |
| | | ATGTACACTCCAGAGCCTGACATCTGCCACGAGCTGCTCGGCCACGTTCCCCTCTTCAGCGACAGGAGCTTCGCGCAGTTCAG |
| | | CCAGGAGATCGGCCTCGCGAGCCTCGGTGCCCCTGACGAGTACATCGAGAAGCTCGCGACGATCTACTGGTTCACGGTCGAGT |
| | | TCGGCCTCTGCAAGCAGGGCGACAGCATCAAGGCGTACGGTGCCGGGCTCCTCAGCAGCTTCGGTGAGCTGCAGTACTGCCTC |
| | | AGCGAGAAGCCCAAGCTCCTTCCACTCGAGCTGGAGAAGACGGCGATCCAGAACTACACGGTCACAGAGTTCCAGCCCCTCT |
| | | ACTACGTCGCGGAGAGCTTCAACGACGCGAAGGAGAAGGTCCGCAACTTCGCTGCCACGATCCCCCGCCCCTTCAGCGTCCGC |
| | | TACGACCCCTACACTCAACGCATCGAAGTCCTCGACAACACTCAACAGCTCAAGATCCTCGCGGACAGCATCAACAGCGAGAT |
| | | CGGCATCCTCTGCAGCGCACTGCAGAAGATCTAG |
| 129 | PAH GS_GCU | ATGTCGACTGCTGTGCTGGAGAACCCGGGCCTGGGCCGCAAGCTCAGCGACTTCGGCCAGGAGACGAGCTACATCGAGGACA |
| | ORF | ACTGCAACCAGAATGGTGCCATCAGCCTGATCTTCAGCCTGAAGGAGGAAGTTGGTGCACTGGCGAAGGTGCTGCGGCTGTTC |
| | | GAGGAGAACGACGTGAATCTCACGCACATCGAGAGCCGCCCCTCACGCCTGAAGAAGGACGAGTACGAGTTCTTCACGCACC |
| | | TGGACAAGCGCAGCCTTCCAGCACTGACGAACATCATCAAGATCCTGCGGCACGACATCGGTGCCACGGTGCACGAGCTCAG |
| | | CCGAGACAAGAAGAAGGACACGGTTCCCTGGTTCCCGCGCACGATCCAGGAGCTGGACCGCTTCGCGAACCAGATCCTCAGC |
| | | TACGGTGCCGAGCTGGACGCGGACCACCCGGGCTTCAAGGACCCGGTGTACCGCGCCCGCCGCAAGCAGTTCGCGGACATCG |
| | | CGTACAACTACCGCCACGGCCAGCCCATCCCCCGCGTGGAGTACATGGAGGAGGAGAAGAAGACGTGGGGCACGGTGTTCAA |
| | | GACGCTGAAGAGCCTGTACAAGACGCACGCGTGCTACGAGTACAACCACATCTTCCCGCTGCTGGAGAAGTACTGTGGGTTCC |
| | | ACGAGGACAACATCCCCCAGCTGGAGGACGTCAGCCAGTTCCTGCAGACGTGCACGGGCTTCCGCCTGCGGCCGGTGGCCGG |
| | | GCTGCTCAGCAGCCGAGACTTCCTGGGTGGCCTGGCGTTCCGCGTGTTCCACTGCACTCAATACATCCGCCACGGCAGCAAGC |
| | | CGATGTACACTCCAGAGCCTGACATCTGCCACGAGCTGCTGGGCCACGTTCCCCTGTTCAGCGACCGCAGCTTCGCGCAGTTC |
| | | AGCCAGGAGATCGGCCTGGCGAGCCTGGGTGCCCCTGACGAGTACATCGAGAAGCTGGCGACGATCTACTGGTTCACGGTGG |
| | | AGTTCGGCCTGTGCAAGCAGGGCGACAGCATCAAGGCGTACGGTGCCGGGCTGCTCAGCAGCTTCGGTGAGCTGCAGTACTG |
| | | CCTCAGCGAGAAGCCGAAGCTGCTTCCACTGGAGCTGGAGAAGACGGCGATCCAGAACTACACGGTGACAGAGTTCCAGCCC |
| | | CTGTACTACGTGGCGGAGAGCTTCAACGACGCGAAGGAGAAGGTGCGCAACTTCGCTGCCACGATCCCCCGCCCGTTCAGCGT |
| | | GCGCTACGACCCGTACACTCAACGCATCGAAGTCCTGGACAACACTCAACAGCTGAAGATCCTGGCGGACAGCATCAACAGC |
| | | GAGATCGGCATCCTGTGCAGCGCACTGCAGAAGATCTAG |
| 130 | TTR amino acid | MASHRLLLLCLAGLVFVSEAGPTGTGESKCPLMVKVLDAVRGSPAINVAVHVFRKAADDTWEPFASGKTSESGELHGL11EEEFVE |
| | sequence | GIYKVEIDTKSYWKALGISPFHEHAEVVFTANDSGPRRYTIAALLSPYSYSTTAVVTNPK |
| 131 | TTR WT ORF | AUGGCUUCUCAUCGUUUAUUAUUAUUAUGUUUAGCUGGUUUAGUUUUUGUUUCUGAAGCUGGUCCUACUGGUACUGGUG |
| | | AAUCUAAAUGUCCUUUAAUGGUUAAAGUUUUAGAUGCUGUUCGUGGUUCUCCUGCUAUUAAUGUUGCUGUUCAUGUUUU |
| | | UCGUAAAGCUGCUGAUGAUACUUGGGAACCUUUUGCUUCUGGUAAAACUUCUGAAUCUGGUGAAUUACAUGGUUUAACU |
| | | ACUGAAGAAGAAUUUGUUGAAGGUAUUUAUAAAGUUGAAAUUGAUACUAAAUCUUAUUGGAAAGCUUUAGGUAUUUCUC |
| | | CUUUUCAUGAACAUGCUGAAGUUGUUUUUACUGCUAAUGAUUCUGGUCCUCGUCGUUAUACUAUUGCUGCUUUAUUAUCU |
| | | CCUUAUUCUUAUUCUACUACUGCUGUUGUUACUAAUCCUAAAUAG |
| 132 | TTR BP GCU | ATGGCGTCGCACCGCCTCCTCCTCCTCTGCCTCGCGGGCCTCGTCTTCGTCTCGGAGGCGGGCCCCACGGGCACGGGCGAGTC |
| | ORF | GAAGTGCCCCCTCATGGTCAAGGTCCTCGACGCGGTCCGCGGCTCGCCCGCGATCAACGTCGCGGTCCACGTCTTCCGCAAGG |
| | | CGGCGGACGACACGTGGGAGCCCTTCGCGTCGGGCAAGACGTCGGAGTCGGGCGAGCTCCACGGCCTCACGACGGAGGAGGA |
| | | GTTCGTCGAGGGCATCTACAAGGTCGAGATCGACACGAAGTCGTACTGGAAGGCGCTCGGCATCTCGCCCTTCCACGAGCACG |
| | | CGGAGGTCGTCTTCACGGCGAACGACTCGGGCCCCCGCCGCTACACGATCGCGGCGCTCCTCTCGCCCTACTCGTACTCGACG |
| | | ACGGCGGTCGTCACGAACCCCAAGTAG |
| 133 | TTR | ATGGCGTCGCACCGCCTCCTCCTCCTCTGCCTCGCCGGGCTCGTCTTCGTCAGCGAAGCCGGGCCCACCGGCACGGGTGAGTC |
| | GP_BP_BS_GCU | GAAGTGCCCCCTCATGGTCAAGGTCCTCGACGCTGTGCGCGGCTCGCCTGCGATCAACGTCGCTGTGCACGTCTTCCGCAAGG |
| | ORF | CTGCCGACGACACGTGGGAGCCCTTCGCGTCGGGCAAGACGTCGGAGTCGGGTGAGCTGCACGGCCTCACGACAGAGGAGGA |
| | | GTTCGTCGAGGGCATCTACAAGGTCGAGATCGACACGAAGTCGTACTGGAAGGCACTGGGCATCTCGCCCTTCCACGAGCACG |
| | | CGGAAGTCGTCTTCACGGCGAACGACTCGGGCCCCCGCCGCTACACGATCGCTGCACTGCTCAGCCCCTACTCGTACTCGACT |
| | | ACGGCTGTGGTCACGAACCCCAAGTAG |
| 134 | TTR | ATGGCGAGCCACCGCCTCCTCCTCCTCTGCCTCGCCGGGCTCGTCTTCGTCAGCGAAGCCGGGCCCACCGGCACGGGTGAGAG |
| | GS_BS_GCU | CAAGTGCCCCCTCATGGTCAAGGTCCTCGACGCTGTGCGCGGCAGCCCTGCGATCAACGTCGCTGTGCACGTCTTCCGCAAGG |
| | ORF | CTGCCGACGACACGTGGGAGCCCTTCGCGAGCGGCAAGACGAGCGAGAGCGGTGAGCTGCACGGCCTCACGACAGAGGAGG |
| | | AGTTCGTCGAGGGCATCTACAAGGTCGAGATCGACACGAAGAGCTACTGGAAGGCACTGGGCATCAGCCCCITCCACGAGCA |
| | | CGCGGAAGTCGTCTTCACGGCGAACGACAGCGGCCCCCGCCGCTACACGATCGCTGCACTGCTCAGCCCCTACAGCTACTCGA |
| | | CTACGGCTGTGGTCACGAACCCCAAGTAG |
| 135 | TTR GS_GCU | ATGGCGAGCCACCGCCTGCTGCTGCTGTGCCTGGCCGGGCTGGTGTTCGTCAGCGAAGCCGGGCCCACCGGCACGGGTGAGA |
| | ORF | GCAAGTGCCCGCTGATGGTGAAGGTGCTGGACGCTGTGCGCGGCAGCCCGGCGATCAACGTGGCTGTGCACGTGTTCCGCAA |
| | | GGCTGCCGACGACACGTGGGAGCCGTTCGCGAGCGGCAAGACGAGCGAGAGCGGTGAGCTGCACGGCCTGACGACAGAGGA |
| | | GGAGTTCGTGGAGGGCATCTACAAGGTGGAGATCGACACGAAGAGCTACTGGAAGGCACTGGGCATCAGCCCGTTCCACGAG |
| | | CACGCGGAAGTCGTGTTCACGGCGAACGACAGCGGCCCGCGCCGCTACACGATCGCTGCACTGCTCAGCCCGTACAGCTACTC |
| | | GACTACGGCTGTGGTGACGAACCCGAAGTAG |
| 136 | FAH BS_GCU | AUGAGCUUCAUCCCCGUCGCGGAGGACAGCGACUUCCCCAUCCACAACCUCCCCUACGGCGUCUUCAGCACGCGCGGCGAC |
| | | CCCCGCCCCCGCAUCGGCGUCGCGAUCGGCGACCAGAUCCUCGACCUCAGCAUCAUCAAGCACCUCUUCACGGGCCCCGUC |
| | | CUCAGCAAGCACCAGGACGUCUUCAACCAGCCCACGCUCAACAGCUUCAUGGGCCUCGGCCAGGCGGCGUGGAAGGAGGCG |
| | | CGCGUCUUCCUCCAGAACCUCCUCAGCGUCAGCCAGGCGCGCCUCCGCGACGACACGGAGCUCCGCAAGUGCGCGUUCAUC |
| | | AGCCAGGCGAGCGCGACGAUGCACCUCCCCGCGACGAUCGGCGACUACACGGACUUCUACAGCAGCCGCCAGCACGCGACG |
| | | AACGUCGGCAUCAUGUUCCGCGACAAGGAGAACGCGCUCAUGCCCAACUGGCUCCACCUCCCCGUCGGCUACCACGGCCGC |
| | | GCGAGCAGCGUCGUCGUCAGCGGCACGCCCAUCCGCCGCCCCAUGGGCCAGAUGAAGCCCGACGACAGCAAGCCCCCCGUC |
| | | UACGGCGCGUGCAAGCUCCUCGACAUGGAGCUCGAGAUGGCGUUCUUCGUCGGCCCCGGCAACCGCCUCGGCGAGCCCAUC |
| | | CCCAUCAGCAAGGCGCACGAGCACAUCUUCGGCAUGGUCCUCAUGAACGACUGGAGCGCGCGCGACAUCCAGAAGUGGGA |
| | | GUACGUCCCCCUCGGCCCCUUCCUCGGCAAGAGCUUCGGCACGACGGUCAGCCCCUGGGUCGUCCCCAUGGACGCGCUCAU |
| | | GCCCUUCGCGGUCCCCAACCCCAAGCAGGACCCCCGCCCCCUCCCCUACCUCUGCCACGACGAGCCCUACACGUUCGACAUC |
| | | AACCUCAGCGUCAACCUCAAGGGCGAGGGCAUGAGCCAGGCGGCGACGAUCUGCAAGAGCAACUUCAAGUACAUGUACUG |
| | | GACGAUGCUCCAGCAGCUCACGCACCACAGCGUCAACGGCUGCAACCUCCGCCCCGGCGACCUCCUCGCGAGCGGCACGAU |
| | | CAGCGGCCCCGAGCCCGAGAACUUCGGCAGCAUGCUCGAGCUCAGCUGGAAGGGCACGAAGCCCAUCGACCUCGGCAACGG |
| | | CCAGACGCGCAAGUUCCUCCUCGACGGCGACGAGGUCAUCAUCACGGGCUACUGCCAGGGCGACGGCUACCGCAUCGGCUU |
| | | CGGCCAGUGCGCGGGCAAGGUCCUCCCCGCGCUCCUCCCCUAG |
| 137 | GABRD | AUGAGCGAGGCGACGCCCCUCGACCGCAACGACAGCGAGAACACGGGCGGCCUCAUCAGCCGCCCCCACCCCUGGGACCAG |
| | BS_GCU | AGCCCCAGCUGCGUCCAGGAGGACCGCGCGAUGAACGACAUCGGCGACUACGUCGGCAGCAACCUCGAGAUCAGCUGGCUC |
| | | CCCAACCUCGACGGCCUCAUCGCGGGCUACGCGCGCAACUUCCGCCCCGGCAUCGGCGGCCCCCCCGUCAACGUCGCGCUC |
| | | GCGCUCGAGGUCGCGAGCAUCGACCACAUCAGCGAGGCGAACAUGGAGUACACGAUGACGGUCUUCCUCCACCAGAGCUG |
| | | GCGCGACAGCCGCCUCAGCUACAACCACACGAACGAGACGCUCGGCCUCGACAGCCGCUUCGUCGACAAGCUCUGGCUCCC |
| | | CGACACGUUCAUCGUCAACGCGAAGAGCGCGUGGUUCCACGACGUCACGGUCGAGAACAAGCUCAUCCGCCUCCAGCCCGA |
| | | CGGCGUCAUCCUCUACAGCAUCCGCAUCACGAGCACGGUCGCGUGCGACAUGGACCUCGCGAAGUACCCCAUGGACGAGCA |
| | | GGAGUGCAUGCUCGACCUCGAGAGCUACGGCUACAGCAGCGAGGACAUCGUCUACUACUGGAGCGAGAGCCAGGAGCACA |
| | | UCCACGGCCUCGACAAGCUCCAGCUCGCGCAGUUCACGAUCACGAGCUACCGCUUCACGACGGAGCUCAUGAACUUCAAGA |
| | | GCGCGGGCCAGUUCCCCCGCCUCAGCCUCCACUUCCACCUCCGCCGCAACCGCGGCGUCUACAUCAUCCAGAGCUACAUGC |
| | | CCAGCGUCCUCCUCGUCGCGAUGAGCUGGGUCAGCUUCUGGAUCAGCCAGGCGGCGGUCCCCGCGCGCGUCAGCCUCGGCA |
| | | UCACGACGGUCCUCACGAUGACGACGCUCAUGGUCAGCGCGCGCAGCAGCCUCCCCCGCGCGAGCGCGAUCAAGGCGCUCG |
| | | ACGUCUACUUCUGGAUCUGCUACGUCUUCGUCUUCGCGGCGCUCGUCGAGUACGCGUUCGCGCACUUCAACGCGGACUACC |
| | | GCAAGAAGCAGAAGGCGAAGGUCAAGGUCAGCCGCCCCCGCGCGGAGAUGGACGUCCGCAACGCGAUCGUCCUCUUCAGC |
| | | CUCAGCGCGGCGGGCGUCACGCAGGAGCUCGCGAUCAGCCGCCGCCAGCGCCGCGUCCCCGGCAACCUCAUGGGCAGCUAC |
| | | CGCAGCGUCGGCGUCGAGACGGGCGAGACGAAGAAGGAGGGCGCGGCGCGCAGCGGCGGCCAGGGCGGCAUCCGCGCGCG |
| | | CCUCCGCCCCAUCGACGCGGACACGAUCGACAUCUACGCGCGCGCGGUCUUCCCCGCGGCGUUCGCGGCGGUCAACGUCAU |
| | | CUACUGGGCGGCGUACGCGUAG |
| 138 | GAPDH | AUGGGCAAGGUCAAGGUCGGCGUCAACGGCUUCGGCCGCAUCGGCCGCCUCGUCACGCGCGCGGCGUUCAACAGCGGCAA |
| | BS_GCU | GGUCGACAUCGUCGCGAUCAACGACCCCUUCAUCGACCUCAACUACAUGGCGGAGAACGGCAAGCUCGUCAUCAACGGCA |
| | | ACCCCAUCACGAUCUUCCAGGAGCGCGACCCCAGCAAGAUCAAGUGGGGCGACGCGGGCGCGGAGUACGUCGUCGAGAGC |
| | | ACGGGCGUCUUCACGACGAUGGAGAAGGCGGGCGCGCACCUCCAGGGCGGCGCGAAGCGCGUCAUCAUCAGCGCGCCCAGC |
| | | GCGGACGCGCCCAUGUUCGUCAUGGGCGUCAACCACGAGAAGUACGACAACAGCCUCAAGAUCAUCAGCAACGCGAGCUG |
| | | CACGACGAACUGCCUCGCGCCCCUCGCGAAGGUCAUCCACGACAACUUCGGCAUCGUCGAGGGCCUCAUGACGACGGUCCA |
| | | CGCGAUCACGGCGACGCAGAAGACGGUCGACGGCCCCAGCGGCAAGCUCUGGCGCGACGGCCGCGGCGCGCUCCAGAACAU |
| | | CAUCCCCGCGAGCACGGGCGCGGCGAAGGCGGUCGGCAAGGUCAUCCCCGAGCUCAACGGCAAGCUCACGGGCAUGGCGUU |
| | | CCGCGUCCCCACGGCGAACGUCAGCGUCGUCGACCUCACGUGCCGCCUCGAGAAGCCCGCGAAGUACGACGACAUCAAGAA |
| | | GGUCGUCAAGCAGGCGAGCGAGGGCCCCCUCAAGGGCAUCCUCGGCUACACGGAGCACCAGGUCGUCAGCAGCGACUUCA |
| | | ACAGCGACACGCACAGCAGCACGUUCGACGCGGGCGCGGGCAUCGCGCUCAACGACCACUUCGUCAAGCUCAUCAGCUGGU |
| | | ACGACAACGAGUUCGGCUACAGCAACCGCGUCGUCGACCUCAUGGCGCACAUGGCGAGCAAGUAG |
| 139 | GBA1 BS_GCU | AUGGAGUUCAGCAGCCCCAGCCGCGAGGAGUGCCCCAAGCCCCUCAGCCGCGUCAGCAUCAUGGCGGGCAGCCUCACGGGC |
| | | CUCCUCCUCCUCCAGGCGGUCAGCUGGGCGAGCGGCGCGCGCCCCUGCAUCCCCAAGAGCUUCGGCUACAGCAGCGUCGUC |
| | | UGCGUCUGCAACGCGACGUACUGCGACAGCUUCGACCCCCCCACGUUCCCCGCGCUCGGCACGUUCAGCCGCUACGAGAGC |
| | | ACGCGCAGCGGCCGCCGCAUGGAGCUCAGCAUGGGCCCCAUCCAGGCGAACCACACGGGCACGGGCCUCCUCCUCACGCUC |
| | | CAGCCCGAGCAGAAGUUCCAGAAGGUCAAGGGCUUCGGCGGCGCGAUGACGGACGCGGCGGCGCUCAACAUCCUCGCGCU |
| | | CAGCCCCCCCGCGCAGAACCUCCUCCUCAAGAGCUACUUCAGCGAGGAGGGCAUCGGCUACAACAUCAUCCGCGUCCCCAU |
| | | GGCGAGCUGCGACUUCAGCAUCCGCACGUACACGUACGCGGACACGCCCGACGACUUCCAGCUCCACAACUUCAGCCUCCC |
| | | CGAGGAGGACACGAAGCUCAAGAUCCCCCUCAUCCACCGCGCGCUCCAGCUCGCGCAGCGCCCCGUCAGCCUCCUCGCGAG |
| | | CCCCUGGACGAGCCCCACGUGGCUCAAGACGAACGGCGCGGUCAACGGCAAGGGCAGCCUCAAGGGCCAGCCCGGCGACAU |
| | | CUACCACCAGACGUGGGCGCGCUACUUCGUCAAGUUCCUCGACGCGUACGCGGAGCACAAGCUCCAGUUCUGGGCGGUCAC |
| | | GGCGGAGAACGAGCCCAGCGCGGGCCUCCUCAGCGGCUACCCCUUCCAGUGCCUCGGCUUCACGCCCGAGCACCAGCGCGA |
| | | CUUCAUCGCGCGCGACCUCGGCCCCACGCUCGCGAACAGCACGCACCACAACGUCCGCCUCCUCAUGCUCGACGACCAGCG |
| | | CCUCCUCCUCCCCCACUGGGCGAAGGUCGUCCUCACGGACCCCGAGGCGGCGAAGUACGUCCACGGCAUCGCGGUCCACUG |
| | | GUACCUCGACUUCCUCGCGCCCGCGAAGGCGACGCUCGGCGAGACGCACCGCCUCUUCCCCAACACGAUGCUCUUCGCGAG |
| | | CGAGGCGUGCGUCGGCAGCAAGUUCUGGGAGCAGAGCGUCCGCCUCGGCAGCUGGGACCGCGGCAUGCAGUACAGCCACA |
| | | GCAUCAUCACGAACCUCCUCUACCACGUCGUCGGCUGGACGGACUGGAACCUCGCGCUCAACCCCGAGGGCGGCCCCAACU |
| | | GGGUCCGCAACUUCGUCGACAGCCCCAUCAUCGUCGACAUCACGAAGGACACGUUCUACAAGCAGCCCAUGUUCUACCACC |
| | | UCGGCCACUUCAGCAAGUUCAUCCCCGAGGGCAGCCAGCGCGUCGGCCUCGUCGCGAGCCAGAAGAACGACCUCGACGCGG |
| | | UCGCGCUCAUGCACCCCGACGGCAGCGCGGUCGUCGUCGUCCUCAACCGCAGCAGCAAGGACGUCCCCCUCACGAUCAAGG |
| | | ACCCCGCGGUCGGCUUCCUCGAGACGAUCAGCCCCGGCUACAGCAUCCACACGUACCUCUGGCGCCGCUAG |
| 140 | GLA BS_GCU | AUGCAGCUCCGCAACCCCGAGCUCCACCUCGGCUGCGCGCUCGCGCUCCGCUUCCUCGCGCUCGUCAGCUGGGACAUCCCC |
| | | GGCGCGCGCGCGCUCGACAACGGCCUCGCGCGCACGCCCACGAUGGGCUGGCUCCACUGGGAGCGCUUCAUGUGCAACCUC |
| | | GACUGCCAGGAGGAGCCCGACAGCUGCAUCAGCGAGAAGCUCUUCAUGGAGAUGGCGGAGCUCAUGGUCAGCGAGGGCUG |
| | | GAAGGACGCGGGCUACGAGUACCUCUGCAUCGACGACUGCUGGAUGGCGCCCCAGCGCGACAGCGAGGGCCGCCUCCAGGC |
| | | GGACCCCCAGCGCUUCCCCCACGGCAUCCGCCAGCUCGCGAACUACGUCCACAGCAAGGGCCUCAAGCUCGGCAUCUACGC |
| | | GGACGUCGGCAACAAGACGUGCGCGGGCUUCCCCGGCAGCUUCGGCUACUACGACAUCGACGCGCAGACGUUCGCGGACU |
| | | GGGGCGUCGACCUCCUCAAGUUCGACGGCUGCUACUGCGACAGCCUCGAGAACCUCGCGGACGGCUACAAGCACAUGAGCC |
| | | UCGCGCUCAACCGCACGGGCCGCAGCAUCGUCUACAGCUGCGAGUGGCCCCUCUACAUGUGGCCCUUCCAGAAGCCCAACU |
| | | ACACGGAGAUCCGCCAGUACUGCAACCACUGGCGCAACUUCGCGGACAUCGACGACAGCUGGAAGAGCAUCAAGAGCAUC |
| | | CUCGACUGGACGAGCUUCAACCAGGAGCGCAUCGUCGACGUCGCGGGCCCCGGCGGCUGGAACGACCCCGACAUGCUCGUC |
| | | AUCGGCAACUUCGGCCUCAGCUGGAACCAGCAGGUCACGCAGAUGGCGCUCUGGGCGAUCAUGGCGGCGCCCCUCUUCAU |
| | | GAGCAACGACCUCCGCCACAUCAGCCCCCAGGCGAAGGCGCUCCUCCAGGACAAGGACGUCAUCGCGAUCAACCAGGACCC |
| | | CCUCGGCAAGCAGGGCUACCAGCUCCGCCAGGGCGACAACUUCGAGGUCUGGGAGCGCCCCCUCAGCGGCCUCGCGUGGGC |
| | | GGUCGCGAUGAUCAACCGCCAGGAGAUCGGCGGCCCCCGCAGCUACACGAUCGCGGUCGCGAGCCUCGGCAAGGGCGUCGC |
| | | GUGCAACCCCGCGUGCUUCAUCACGCAGCUCCUCCCCGUCAAGCGCAAGCUCGGCUUCUACGAGUGGACGAGCCGCCUCCG |
| | | CAGCCACAUCAACCCCACGGGCACGGUCCUCCUCCAGCUCGAGAACACGAUGCAGAUGAGCCUCAAGGACCUCUAG |
| 141 | OTC BS_GCU | AUGCUCUUCAACCUCCGCAUCCUCCUCAACAACGCGGCGUUCCGCAACGGCCACAACUUCAUGGUCCGCAACUUCCGCUGC |
| | | GGCCAGCCCCUCCAGAACAAGGUCCAGCUCAAGGGCCGCGACCUCCUCACGCUCAAGAACUUCACGGGCGAGGAGAUCAAG |
| | | UACAUGCUCUGGCUCAGCGCGGACCUCAAGUUCCGCAUCAAGCAGAAGGGCGAGUACCUCCCCCUCCUCCAGGGCAAGAGC |
| | | CUCGGCAUGAUCUUCGAGAAGCGCAGCACGCGCACGCGCCUCAGCACGGAGACGGGCUUCGCGCUCCUCGGCGGCCACCCC |
| | | UGCUUCCUCACGACGCAGGACAUCCACCUCGGCGUCAACGAGAGCCUCACGGACACGGCGCGCGUCCUCAGCAGCAUGGCG |
| | | GACGCGGUCCUCGCGCGCGUCUACAAGCAGAGCGACCUCGACACGCUCGCGAAGGAGGCGAGCAUCCCCAUCAUCAACGGC |
| | | CUCAGCGACCUCUACCACCCCAUCCAGAUCCUCGCGGACUACCUCACGCUCCAGGAGCACUACAGCAGCCUCAAGGGCCUC |
| | | ACGCUCAGCUGGAUCGGCGACGGCAACAACAUCCUCCACAGCAUCAUGAUGAGCGCGGCGAAGUUCGGCAUGCACCUCCA |
| | | GGCGGCGACGCCCAAGGGCUACGAGCCCGACGCGAGCGUCACGAAGCUCGCGGAGCAGUACGCGAAGGAGAACGGCACGA |
| | | AGCUCCUCCUCACGAACGACCCCCUCGAGGCGGCGCACGGCGGCAACGUCCUCAUCACGGACACGUGGAUCAGCAUGGGCC |
| | | AGGAGGAGGAGAAGAAGAAGCGCCUCCAGGCGUUCCAGGGCUACCAGGUCACGAUGAAGACGGCGAAGGUCGCGGCGAGC |
| | | GACUGGACGUUCCUCCACUGCCUCCCCCGCAAGCCCGAGGAGGUCGACGACGAGGUCUUCUACAGCCCCCGCAGCCUCGUC |
| | | UUCCCCGAGGCGGAGAACCGCAAGUGGACGAUCAUGGCGGUCAUGGUCAGCCUCCUCACGGACUACAGCCCCCAGCUCCAG |
| | | AAGCCCAAGUAG |
| 142 | PAH BS_GCU | AUGAGCACGGCGGUCCUCGAGAACCCCGGCCUCGGCCGCAAGCUCAGCGACUUCGGCCAGGAGACGAGCUACAUCGAGGAC |
| | | AACUGCAACCAGAACGGCGCGAUCAGCCUCAUCUUCAGCCUCAAGGAGGAGGUCGGCGCGCUCGCGAAGGUCCUCCGCCUC |
| | | UUCGAGGAGAACGACGUCAACCUCACGCACAUCGAGAGCCGCCCCAGCCGCCUCAAGAAGGACGAGUACGAGUUCUUCAC |
| | | GCACCUCGACAAGCGCAGCCUCCCCGCGCUCACGAACAUCAUCAAGAUCCUCCGCCACGACAUCGGCGCGACGGUCCACGA |
| | | GCUCAGCCGCGACAAGAAGAAGGACACGGUCCCCUGGUUCCCCCGCACGAUCCAGGAGCUCGACCGCUUCGCGAACCAGAU |
| | | CCUCAGCUACGGCGCGGAGCUCGACGCGGACCACCCCGGCUUCAAGGACCCCGUCUACCGCGCGCGCCGCAAGCAGUUCGC |
| | | GGACAUCGCGUACAACUACCGCCACGGCCAGCCCAUCCCCCGCGUCGAGUACAUGGAGGAGGAGAAGAAGACGUGGGGCA |
| | | CGGUCUUCAAGACGCUCAAGAGCCUCUACAAGACGCACGCGUGCUACGAGUACAACCACAUCUUCCCCCUCCUCGAGAAGU |
| | | ACUGCGGCUUCCACGAGGACAACAUCCCCCAGCUCGAGGACGUCAGCCAGUUCCUCCAGACGUGCACGGGCUUCCGCCUCC |
| | | GCCCCGUCGCGGGCCUCCUCAGCAGCCGCGACUUCCUCGGCGGCCUCGCGUUCCGCGUCUUCCACUGCACGCAGUACAUCC |
| | | GCCACGGCAGCAAGCCCAUGUACACGCCCGAGCCCGACAUCUGCCACGAGCUCCUCGGCCACGUCCCCCUCUUCAGCGACC |
| | | GCAGCUUCGCGCAGUUCAGCCAGGAGAUCGGCCUCGCGAGCCUCGGCGCGCCCGACGAGUACAUCGAGAAGCUCGCGACGA |
| | | UCUACUGGUUCACGGUCGAGUUCGGCCUCUGCAAGCAGGGCGACAGCAUCAAGGCGUACGGCGCGGGCCUCCUCAGCAGC |
| | | UUCGGCGAGCUCCAGUACUGCCUCAGCGAGAAGCCCAAGCUCCUCCCCCUCGAGCUCGAGAAGACGGCGAUCCAGAACUAC |
| | | ACGGUCACGGAGUUCCAGCCCCUCUACUACGUCGCGGAGAGCUUCAACGACGCGAAGGAGAAGGUCCGCAACUUCGCGGC |
| | | GACGAUCCCCCGCCCCUUCAGCGUCCGCUACGACCCCUACACGCAGCGCAUCGAGGUCCUCGACAACACGCAGCAGCUCAA |
| | | GAUCCUCGCGGACAGCAUCAACAGCGAGAUCGGCAUCCUCUGCAGCGCGCUCCAGAAGAUCUAG |
| 143 | TTR BS_GCU | AUGGCGAGCCACCGCCUCCUCCUCCUCUGCCUCGCGGGCCUCGUCUUCGUCAGCGAGGCGGGCCCCACGGGCACGGGCGAG |
| | | AGCAAGUGCCCCCUCAUGGUCAAGGUCCUCGACGCGGUCCGCGGCAGCCCCGCGAUCAACGUCGCGGUCCACGUCUUCCGC |
| | | AAGGCGGCGGACGACACGUGGGAGCCCUUCGCGAGCGGCAAGACGAGCGAGAGCGGCGAGCUCCACGGCCUCACGACGGA |
| | | GGAGGAGUUCGUCGAGGGCAUCUACAAGGUCGAGAUCGACACGAAGAGCUACUGGAAGGCGCUCGGCAUCAGCCCCUUCC |
| | | ACGAGCACGCGGAGGUCGUCUUCACGGCGAACGACAGCGGCCCCCGCCGCUACACGAUCGCGGCGCUCCUCAGCCCCUACA |
| | | GCUACAGCACGACGGCGGUCGUCACGAACCCCAAGUAG |
| 144-160 | | Not Used |
| 161 | Cas9 nickase | MDKKYSIGLAIGTNSVGWAVITDEYKVPSKKFKVLGNTDRHSIKKNLIGALLFDSGETAEATRLKRTARRRYTRRKNRICYLQEIFS |
| | amino acid | NEMAKVDDSFFHRLEESFLVEEDKKHERHPIFGNIVDEVAYHEKYPTIYHLRKKLVDSTDKADLRLIYLALAHMIKFRGHFLIEGDL |
| | sequence | NPDNSDVDKLFIQLVQTYNQLFEENPINASGVDAKAILSARLSKSRRLENLIAQLPGEKKNGLFGNLIALSLGLTPNFKSNFDLAEDA |
| | | KLQLSKDTYDDDLDNLLAQIGDQYADLFLAAKNLSDAILLSDILRVNTEITKAPLSASMIKRYDEHHQDLTLLKALVRQQLPEKYKE |
| | | IFFDQSKNGYAGYIDGGASQEEFYKFIKPILEKMDGTEELLVKLNREDLLRKQRTFDNGSIPHQIHLGELHAILRRQEDFYPFLKDNRE |
| | | KIEKILTFRIPYYVGPLARGNSRFAWMTRKSEETITPWNFEEVVDKGASAQSFIERMTNFDKNLPNEKVLPKHSLLYEYFTVYNELTK |
| | | VKYVTEGMRKPAFLSGEQKKAIVDLLFKTNRKVTVKQLKEDYFKKIECFDSVEISGVEDRFNASLGTYHDLLKIIKDKDFLDNEENE |
| | | DILEDIVLTLTLFEDREMIEERLKTYAHLFDDKVMKQLKRRRYTGWGRLSRKLINGIRDKQSGKTILDFLKSDGFANRNFMQLIHDD |
| | | SLTFKEDIQKAQVSGQGDSLHEHIANLAGSPAIKKGILQTVKVVDELVKVMGRHKPENIVIEMARENQTTQKGQKNSRERMKRIEE |
| | | GIKELGSQILKEHPVENTQLQNEKLYLYYLQNGRDMYVDQELDINRLSDYDVDHIVPQSFLKDDSIDNKVLTRSDKNRGKSDNVPS |
| | | EEVVKKMKNYWRQLLNAKLITQRKFDNLTKAERGGLSELDKAGFIKRQLVETRQITKHVAQILDSRMNTKYDENDKLIREVKVITL |
| | | KSKLVSDFRKDFQFYKVREINNYHHAHDAYLNAVVGTALIKKYPKLESEFVYGDYKVYDVRKMIAKSEQEIGKATAKYFFYSNIM |
| | | NFFKTEITLANGEIRKRPLIETNGETGEIVWDKGRDFATVRKVLSMPQVNIVKKTEVQTGGFSKESILPKRNSDKLIARKKDWDPKK |
| | | YGGFDSPTVAYSVLVVAKVEKGKSKKLKSVKELLGITIMERSSEEKNPIDFLEAKGYKEVKKDLIIKLPKYSLFELENGRKRMLASA |
| | | GELQKGNELALPSKYVNFLYLASHYEKLKGSPEDNEQKQLFVEQHKHYLDEIIEQISEFSKRVILADANLDKVLSAYNKHRDKPIRE |
| | | QAENIIHLFTLTNLGAPAAFKYFDTTIDRKRYTSTKEVLDATLIHQSITGLYETRIDLSQLGGDGGGSPKKKRKV |
| 162 | dCas9 amino | MDKKYSIGLAIGTNSVGWAVITDEYKVPSKKFKVLGNTDRHSIKKNLIGALLFDSGETAEATRLKRTARRRYTRRKNRICYLQEIFS |
| | acid sequence | NEMAKVDDSFFHRLEESFLVEEDKKHERHPIFGNIVDEVAYHEKYPTIYHLRKKLVDSTDKADLRLIYLALAHMIKFRGHFLIEGDL |
| | | NPDNSDVDKLFIQLVQTYNQLFEENPINASGVDAKAILSARLSKSRRLENLIAQLPGEKKNGLFGNLIALSLGLTPNFKSNFDLAEDA |
| | | KLQLSKDTYDDDLDNLLAQIGDQYADLFLAAKNLSDAILLSDILRVNTEITKAPLSASMIKRYDEHHQDLTLLKALVRQQLPEKYKE |
| | | IFFDQSKNGYAGYIDGGASQEEFYKFIKPILEKMDGTEELLVKLNREDLLRKQRTFDNGSIPHQIHLGELHAILRRQEDFYPFLKDNRE |
| | | KIEKILTFRIPYYVGPLARGNSRFAWMTRKSEETITPWNFEEVVDKGASAQSFIERMTNFDKNLPNEKVLPKHSLLYEYFTVYNELTK |
| | | VKYVTEGMRKPAFLSGEQKKAIVDLLFKTNRKVTVKQLKEDYFKKIECFDSVEISGVEDRFNASLGTYHDLLKIIKDKDFLDNEENE |
| | | DILEDIVLTLTLFEDREMIEERLKTYAHLFDDKVMKQLKRRRYTGWGRLSRKLINGIRDKQSGKTILDFLKSDGFANRNFMQLIHDD |
| | | SLTFKEDIQKAQVSGQGDSLHEHIANLAGSPAIKKGILQTVKVVDELVKVMGRHKPENIVIEMARENQTTQKGQKNSRERMKRIEE |
| | | GIKELGSQILKEHPVENTQLQNEKLYLYYLQNGRDMYVDQELDINRLSDYDVDAIVPQSFLKDDSIDNKVLTRSDKNRGKSDNVPS |
| | | EEVVKKMKNYWRQLLNAKLITQRKFDNLTKAERGGLSELDKAGFIKRQLVETRQITKHVAQILDSRMNTKYDENDKLIREVKVITL |
| | | KSKLVSDFRKDFQFYKVREINNYHHAHDAYLNAVVGTALIKKYPKLESEFVYGDYKVYDVRKMIAKSEQEIGKATAKYFFYSNIM |
| | | NFFKTEITLANGEIRKRPLIETNGETGEIVWDKGRDFATVRKVLSMPQVNIVKKTEVQTGGFSKESILPKRNSDKLIARKKDWDPKK |
| | | YGGFDSPTVAYSVLVVAKVEKGKSKKLKSVKELLGITIMERSSEEKNPIDFLEAKGYKEVKKDLIIKLPKYSLFELENGRKRMLASA |
| | | GELQKGNELALPSKYVNFLYLASHYEKLKGSPEDNEQKQLFVEQHKHYLDEIIEQISEFSKRVILADANLDKVLSAYNKHRDKPIRE |
| | | QAENIIHLFTLTNLGAPAAFKYFDTTIDRKRYTSTKEVLDATLIHQSITGLYETRIDLSQLGGDGGGSPKKKRKV |
| 163 | Exemplary NLS | PKKKRKV |
| | amino acid | |
| | sequence | |
| 164 | Exemplary NLS | LAAKRSRTT |
| | amino acid | |
| | sequence | |
| 165 | Exemplary NLS | QAAKRSRTT |
| | amino acid | |
| | sequence | |
| 166 | Exemplary NLS | PAPAKRERTT |
| | amino acid | |
| | sequence | |
| 167 | Exemplary NLS | QAAKRPRTT |
| | amino acid | |
| | sequence | |
| 168 | Exemplary NLS | RAAKRPRTT |
| | amino acid | |
| | sequence | |
| 169 | Exemplary NLS | AAAKRSWSMAA |
| | amino acid | |
| | sequence | |
| 170 | Exemplary NLS | AAAKRVWSMAF |
| | amino acid | |
| | sequence | |
| 171 | Exemplary NLS | AAAKRSWSMAF |
| | amino acid | |
| | sequence | |
| 172 | Exemplary NLS | AAAKRKYFAA |
| | amino acid | |
| | sequence | |
| 173 | Exemplary NLS | RAAKRKAFAA |
| | amino acid | |
| | sequence | |
| 174 | Exemplary NLS | RAAKRKYFAV |
| | amino acid | |
| | sequence | |
| 175 | Exemplary NLS | PKKKRRV |
| | amino acid | |
| | sequence | |
| 176 | Exemplary NLS | KRPAATKKAGQAKKKK |
| | amino acid | |
| | sequence | |
| 177 | Exemplary 5′ | ACATTTGCTTCTGACACAACTGTGTTCACTAGCAACCTCAAACAGACACC |
| | UTR | |
| 178 | Exemplary 5′ | CATAAACCCTGGCGCGCTCGCGGCCCGGCACTCTTCTGGTCCCCACAGACTCAGAGAGAACCCACC |
| | UTR | |
| 179 | Exemplary 5′ | AAGCTCAGAATAAACGCTCAACTTTGGCC |
| | UTR | |
| 180 | Exemplary 5′ | CAGGGTCCTGTGGACAGCTCACCAGCT |
| | UTR | |
| 181 | Exemplary 5′ | TCCCGCAGTCGGCGTCCAGCGGCTCTGCTTGTTCGTGTGTGTGTCGTTGCAGGCCTTATTC |
| | UTR | |
| 182 | Exemplary 3′ | GCTCGCTTTCTTGCTGTCCAATTTCTATTAAAGGTTCCTTTGTTCCCTAAGTCCAACTACTAAACTGGGGGATATTATGAAGGG |
| | UTR | CCTTGAGCATCTGGATTCTGCCTAATAAAAAACATTTATTTTCATTGC |
| 183 | Exemplary 3′ | GCTGGAGCCTCGGTGGCCATGCTTCTTGCCCCTTGGGCCTCCCCCCAGCCCCTCCTCCCCTTCCTGCACCCGTACCCCCGTGGTC |
| | UTR | TTTGAATAAAGTCTGAGTGGGCGGC |
| 184 | Exemplary 3′ | ACCAGCCTCAAGAACACCCGAATGGAGTCTCTAAGCTACATAATACCAACTTACACTTTACAAAATGTTGTCCCCCAAAATGT |
| | UTR | AGCCATTCGTATCTGCTCCTAATAAAAAGAAAGTTTCTTCACATTCT |
| 185 | Exemplary 3′ | TTGCCAGCCATCTGTTGTTTGCCCCTCCCCCGTGCCTTCCTTGACCCTGGAAGGTGCCACTCCCACTGTCCTTTCCTAATAAAAT |
| | UTR | GAGGAAATTGCATCGCA |
| 186 | Exemplary 3′ | GCTGCCTTCTGCGGGGCTTGCCTTCTGGCCATGCCCTTCTTCTCTCCCTTGCACCTGTACCTCTTGGTCTTTGAATAAAGCCTGA |
| | UTR | GTAGGAAG |
| 187 | Exemplary | gccgccRccAUGG |
| | Kozak sequence | |
| 188 | Exemplary poly- | AAAAAAAAAAAAAAAAAAAAAAAAAAAAAAGCGAAAAAAAAAAAAAAAAAAAAAAAAAAAAAACCGAAAAAAAAAAAAA |
| | A sequence | AAAAAAAAAAAAAAAAAAAAAAAAAA |
| 189 | Exemplary guide | mN*mN*mN*NNNNNNNNNNNNNNNNNGUUUUAG |
| | pattern | AmGmCmUmAmGmAmAmAmUmAmGmCAAGUUAAA |
| | | AUAAGGCUAGUCCGUUAUCAmAmCmUmUmGmAm |
| | | AmAmAmAmGmUmGmGmCmAmCmCmGmAmGmUmC |
| | | mGmGmUmGmCmU*mU*mU*mU |
| 190 | Exemplary 5′ | CAGATCGCCTGGAGACGCCATCCACGCTGTTTTGACCTCCAT |
| | UTR | |
| 191 | Exemplary 5′ | AGAAGACACCGGGACCGATCCAGCCTCCGCGGCCGGGAACGG |
| | UTR | |
| 192 | Exemplary 5′ | TGCATTGGAACGCGGATTCCCCGTGCCAAGAGTGACTCACCG |
| | UTR | |
| 193 | Cas9 mRNA | GGGAAGCUCAGAAUAAACGCUCAACUUUGGCCGGAUCUGCCACCAUGGACAAGAAGUACAGCAUCGGACUGGACAUCGGA |
| | transcript | ACAAACAGCGUUGGCUGGGCUGUGAUCACAGACGAAUACAAGGUUCCCUCAAAGAAGUUCAAGGUCCUGGGAAACACAGA |
| | comprising SEQ | CAGACACAGCAUCAAGAAGAAUCUCAUCGGUGCACUGCUGUUCGACAGCGGUGAGACAGCAGAAGCCACAAGACUGAAGA |
| | 29 | GAACAGCCCGCAGAAGAUACACAAGAAGAAAGAACAGAAUCUGCUACCUGCAGGAGAUCUUCAGCAACGAAAUGGCAAAG |
| | | GUCGACGACAGCUUCUUCCACAGACUGGAAGAAAGCUUCCUGGUCGAAGAAGACAAGAAGCACGAAAGACACCCGAUCUU |
| | | CGGAAACAUCGUCGACGAAGUCGCAUACCACGAAAAGUACCCCACCAUCUACCACCUGCGGAAGAAGCUGGUCGACUCGA |
| | | CUGACAAGGCAGACCUGCGGCUGAUCUACCUGGCACUGGCACACAUGAUAAAGUUCAGAGGACACUUCCUGAUCGAAGGA |
| | | GACCUGAACCCUGACAACAGCGACGUCGACAAGCUGUUCAUCCAGCUGGUCCAGACCUACAACCAGCUGUUCGAAGAAAA |
| | | CCCGAUCAACGCAAGCGGAGUCGACGCAAAGGCAAUCCUCAGCGCCCGCCUCAGCAAGAGCAGAAGACUGGAAAAUCUCA |
| | | UCGCACAGCUUCCAGGUGAGAAGAAGAAUGGGCUGUUCGGAAAUCUCAUCGCACUCAGCCUGGGACUGACUCCCAACUUC |
| | | AAGAGCAACUUCGACCUGGCAGAAGACGCAAAGCUGCAGCUCAGCAAGGACACCUACGACGACGACCUGGACAAUCUCCU |
| | | GGCACAGAUCGGAGACCAGUACGCAGACCUGUUCCUGGCUGCCAAGAAUCUCAGCGACGCAAUCCUGCUCAGCGACAUCC |
| | | UGCGGGUCAACACAGAGAUCACAAAGGCACCGCUCAGCGCAAGCAUGAUAAAGAGAUACGACGAACACCACCAGGACCUG |
| | | ACACUGCUGAAGGCACUGGUCAGACAGCAGCUUCCAGAGAAGUACAAGGAAAUCUUCUUCGACCAGAGCAAGAAUGGGUA |
| | | CGCCGGGUACAUCGACGGUGGUGCCAGCCAGGAGGAAUUCUACAAGUUCAUCAAGCCGAUCCUGGAAAAGAUGGACGGAA |
| | | CAGAGGAGCUGCUGGUCAAGCUGAACAGGGAGGACCUGCUGCGGAAGCAGAGAACAUUCGACAAUGGGAGCAUCCCCCAC |
| | | CAGAUCCACCUGGGUGAGCUGCACGCAAUCCUGCGGAGACAGGAGGACUUCUACCCGUUCCUGAAGGACAACAGGGAGAA |
| | | GAUCGAAAAGAUCCUGACAUUCAGAAUCCCCUACUACGUUGGCCCGCUGGCCCGCGGAAACAGCAGAUUCGCAUGGAUGA |
| | | CAAGAAAGAGCGAAGAAACAAUCACUCCCUGGAACUUCGAAGAAGUCGUCGACAAGGGUGCCAGCGCACAGAGCUUCAUC |
| | | GAAAGAAUGACAAACUUCGACAAGAAUCUUCCAAACGAAAAGGUCCUUCCAAAGCACAGCCUGCUGUACGAAUACUUCAC |
| | | AGUCUACAACGAGCUGACAAAGGUCAAGUACGUCACAGAGGGAAUGAGAAAGCCGGCAUUCCUCAGCGGUGAGCAGAAGA |
| | | AGGCAAUCGUCGACCUGCUGUUCAAGACAAACAGAAAGGUCACAGUCAAGCAGCUGAAGGAAGACUACUUCAAGAAGAUC |
| | | GAAUGCUUCGACAGCGUCGAAAUCAGCGGAGUCGAAGACAGAUUCAACGCAAGCCUGGGAACCUACCACGACCUGCUGAA |
| | | GAUCAUCAAGGACAAGGACUUCCUGGACAACGAAGAAAACGAAGACAUCCUGGAAGACAUCGUCCUGACACUGACACUGU |
| | | UCGAAGACAGGGAGAUGAUAGAAGAAAGACUGAAGACCUACGCACACCUGUUCGACGACAAGGUCAUGAAGCAGCUGAAG |
| | | AGAAGAAGAUACACAGGAUGGGGAAGACUCAGCAGAAAGCUGAUCAAUGGGAUCCGAGACAAGCAGAGCGGAAAGACAA |
| | | UCCUGGACUUCCUGAAGAGCGACGGAUUCGCAAACAGAAACUUCAUGCAGCUGAUCCACGACGACAGCCUGACAUUCAAG |
| | | GAAGACAUCCAGAAGGCACAGGUCAGCGGACAGGGCGACAGCCUGCACGAACACAUCGCAAAUCUCGCCGGGAGCCCGGC |
| | | AAUCAAGAAGGGGAUCCUGCAGACAGUCAAGGUCGUCGACGAGCUGGUCAAGGUCAUGGGAAGACACAAGCCAGAGAACA |
| | | UCGUCAUCGAAAUGGCCAGGGAGAACCAGACAACUCAAAAGGGGCAGAAGAACAGCAGGGAGAGAAUGAAGAGAAUCGA |
| | | AGAAGGAAUCAAGGAGCUGGGAAGCCAGAUCCUGAAGGAACACCCGGUCGAAAACACUCAACUGCAGAACGAAAAGCUGU |
| | | ACCUGUACUACCUGCAGAAUGGGCGAGACAUGUACGUCGACCAGGAGCUGGACAUCAACAGACUCAGCGACUACGACGUC |
| | | GACCACAUCGUUCCCCAGAGCUUCCUGAAGGACGACAGCAUCGACAACAAGGUCCUGACAAGAAGCGACAAGAACAGAGG |
| | | AAAGAGCGACAACGUUCCCUCAGAAGAAGUCGUCAAGAAGAUGAAGAACUACUGGAGACAGCUGCUGAACGCAAAGCUGA |
| | | UCACUCAAAGAAAGUUCGACAAUCUCACAAAGGCAGAAAGAGGUGGCCUCAGCGAGCUGGACAAGGCCGGGUUCAUCAAG |
| | | AGACAGCUGGUCGAAACAAGACAGAUCACAAAGCACGUCGCACAGAUCCUGGACAGCAGAAUGAACACAAAGUACGACGA |
| | | AAACGACAAGCUGAUCAGGGAAGUCAAGGUCAUCACACUGAAGAGCAAGCUGGUCAGCGACUUCAGAAAGGACUUCCAGU |
| | | UCUACAAGGUCAGGGAGAUCAACAACUACCACCACGCACACGACGCAUACCUGAACGCUGUGGUUGGCACAGCACUGAUC |
| | | AAGAAGUACCCGAAGCUGGAAAGCGAAUUCGUCUACGGAGACUACAAGGUCUACGACGUCAGAAAGAUGAUAGCAAAGAG |
| | | CGAACAGGAGAUCGGAAAGGCAACAGCAAAGUACUUCUUCUACAGCAACAUCAUGAACUUCUUCAAGACAGAGAUCACAC |
| | | UGGCAAAUGGUGAGAUCAGAAAGAGACCGCUGAUCGAAACAAAUGGUGAGACAGGUGAGAUCGUCUGGGACAAGGGGCG |
| | | AGACUUCGCAACAGUCAGAAAGGUCCUCAGCAUGCCGCAGGUGAACAUCGUCAAGAAGACAGAAGUCCAGACAGGUGGCU |
| | | UCAGCAAGGAAAGCAUCCUUCCAAAGAGAAACAGCGACAAGCUGAUCGCCCGCAAGAAGGACUGGGACCCGAAGAAGUAC |
| | | GGUGGCUUCGACAGCCCCACCGUCGCAUACAGCGUCCUGGUCGUCGCAAAGGUCGAAAAGGGGAAGAGCAAGAAGCUGAA |
| | | GAGCGUCAAGGAGCUGCUGGGAAUCACAAUCAUGGAAAGAAGCAGCUUCGAAAAGAACCCGAUCGACUUCCUGGAAGCCA |
| | | AGGGGUACAAGGAAGUCAAGAAGGACCUGAUCAUCAAGCUUCCAAAGUACAGCCUGUUCGAGCUGGAAAAUGGGAGAAA |
| | | GAGAAUGCUGGCAAGCGCCGGUGAGCUGCAGAAGGGGAACGAGCUGGCACUUCCCUCAAAGUACGUCAACUUCCUGUACC |
| | | UGGCAAGCCACUACGAAAAGCUGAAGGGGAGCCCAGAGGACAACGAACAGAAGCAGCUGUUCGUCGAACAGCACAAGCAC |
| | | UACCUGGACGAAAUCAUCGAACAGAUCAGCGAAUUCAGCAAGAGAGUCAUCCUGGCAGACGCAAAUCUCGACAAGGUCCU |
| | | CAGCGCAUACAACAAGCACCGAGACAAGCCGAUCAGGGAGCAGGCCGAAAACAUCAUCCACCUGUUCACACUGACAAAUC |
| | | UCGGUGCCCCGGCUGCCUUCAAGUACUUCGACACAACAAUCGACAGAAAGAGAUACACAUCGACUAAGGAAGUCCUGGAC |
| | | GCAACACUGAUCCACCAGAGCAUCACAGGACUGUACGAAACAAGAAUCGACCUCAGCCAGCUGGGUGGCGACGGUGGUGG |
| | | CAGCCCGAAGAAGAAGAGAAAGGUCUAGCUAGCACCAGCCUCAAGAACACCCGAAUGGAGUCUCUAAGCUACAUAAUACC |
| | | AACUUACACUUUACAAAAUGUUGUCCCCCAAAAUGUAGCCAUUCGUAUCUGCUCCUAAUAAAAAGAAAGUUUCUUCACAU |
| | | UCUCUCGAGAAAAAAAAAAAAAAAAAAAAAAAAAAAAAAAAAAAAAAAAAAAAAAAAAAAAAAAAAAAAAAAAAAAAAA |
| | | AAAAAAAAAAAAAAAAAAAAAAAUCUAG |
| 194 | Cas9 mRNA | GGGAAGCUCAGAAUAAACGCUCAACUUUGGCCGGAUCUGCCACCAUGGACAAGAAGUACAGCAUCGGCCUGGACAUCGGC |
| | transcript | ACCAACUCCGUUGGCUGGGCUGUGAUCACCGACGAGUACAAGGUUCCCUCAAAGAAGUUCAAGGUGCUGGGCAACACCGA |
| | comprising SEQ | CCGGCACAGCAUCAAGAAGAAUCUCAUCGGUGCACUGCUGUUCGACUCCGGUGAGACCGCCGAAGCCACCCGGCUGAAGC |
| | 46 | GGACCGCCCGCCGGCGGUACACCCGGCGGAAGAACCGGAUCUGCUACCUGCAGGAGAUCUUCUCCAACGAGAUGGCCAAG |
| | | GUGGACGACUCCUUCUUCCACCGGCUGGAGGAGUCCUUCCUGGUGGAGGAGGACAAGAAGCACGAGCGGCACCCCAUCUU |
| | | CGGCAACAUCGUGGACGAAGUCGCCUACCACGAGAAGUACCCCACCAUCUACCACCUGCGGAAGAAGCUGGUGGACUCGA |
| | | CUGACAAGGCCGACCUGCGGCUGAUCUACCUGGCACUGGCCCACAUGAUAAAGUUCCGGGGCCACUUCCUGAUCGAGGGC |
| | | GACCUGAACCCUGACAACUCCGACGUGGACAAGCUGUUCAUCCAGCUGGUGCAGACCUACAACCAGCUGUUCGAGGAGAA |
| | | CCCCAUCAACGCCUCCGGCGUGGACGCCAAGGCCAUCCUCAGCGCCCGCCUCAGCAAGUCCCGGCGGCUGGAGAAUCUCAU |
| | | CGCCCAGCUUCCAGGUGAGAAGAAGAAUGGGCUGUUCGGCAAUCUCAUCGCACUCAGCCUGGGCCUGACUCCCAACUUCA |
| | | AGUCCAACUUCGACCUGGCCGAGGACGCCAAGCUGCAGCUCAGCAAGGACACCUACGACGACGACCUGGACAAUCUCCUGG |
| | | CCCAGAUCGGCGACCAGUACGCCGACCUGUUCCUGGCUGCCAAGAAUCUCAGCGACGCCAUCCUGCUCAGCGACAUCCUGC |
| | | GGGUGAACACAGAGAUCACCAAGGCCCCCCUCAGCGCCUCCAUGAUAAAGCGGUACGACGAGCACCACCAGGACCUGACCC |
| | | UGCUGAAGGCACUGGUGCGGCAGCAGCUUCCAGAGAAGUACAAGGAGAUCUUCUUCGACCAGAGCAAGAAUGGGUACGCC |
| | | GGGUACAUCGACGGUGGUGCCUCCCAGGAGGAGUUCUACAAGUUCAUCAAGCCCAUCCUGGAGAAGAUGGACGGCACAGA |
| | | GGAGCUGCUGGUGAAGCUGAACAGGGAGGACCUGCUGCGGAAGCAGCGGACCUUCGACAAUGGGAGCAUCCCCCACCAGA |
| | | UCCACCUGGGUGAGCUGCACGCCAUCCUGCGGCGGCAGGAGGACUUCUACCCCUUCCUGAAGGACAACAGGGAGAAGAUC |
| | | GAGAAGAUCCUGACCUUCCGGAUCCCCUACUACGUUGGCCCCCUGGCCCGCGGCAACUCCCGGUUCGCCUGGAUGACCCGG |
| | | AAGUCCGAGGAGACCAUCACUCCCUGGAACUUCGAGGAAGUCGUGGACAAGGGUGCCUCCGCCCAGAGCUUCAUCGAGCG |
| | | GAUGACCAACUUCGACAAGAAUCUUCCAAACGAGAAGGUGCUUCCAAAGCACUCCCUGCUGUACGAGUACUUCACCGUGU |
| | | ACAACGAGCUGACCAAGGUGAAGUACGUGACAGAGGGCAUGCGGAAGCCCGCCUUCCUCAGCGGUGAGCAGAAGAAGGCC |
| | | AUCGUGGACCUGCUGUUCAAGACCAACCGGAAGGUGACCGUGAAGCAGCUGAAGGAGGACUACUUCAAGAAGAUCGAGUG |
| | | CUUCGACUCCGUGGAGAUCUCCGGCGUGGAGGACCGGUUCAACGCCUCCCUGGGCACCUACCACGACCUGCUGAAGAUCAU |
| | | CAAGGACAAGGACUUCCUGGACAACGAGGAGAACGAGGACAUCCUGGAGGACAUCGUGCUGACCCUGACCCUGUUCGAGG |
| | | ACAGGGAGAUGAUAGAGGAGCGGCUGAAGACCUACGCCCACCUGUUCGACGACAAGGUGAUGAAGCAGCUGAAGCGGCGG |
| | | CGGUACACCGGCUGGGGCCGGCUCAGCCGGAAGCUGAUCAAUGGGAUCCGAGACAAGCAGAGCGGCAAGACCAUCCUGGA |
| | | CUUCCUGAAGUCCGACGGCUUCGCCAACCGGAACUUCAUGCAGCUGAUCCACGACGACUCCCUGACCUUCAAGGAGGACAU |
| | | CCAGAAGGCCCAGGUCAGCGGCCAGGGCGACUCCCUGCACGAGCACAUCGCCAAUCUCGCCGGGUCCCCCGCCAUCAAGAA |
| | | GGGGAUCCUGCAGACCGUGAAGGUGGUGGACGAGCUGGUGAAGGUGAUGGGCCGGCACAAGCCAGAGAACAUCGUGAUCG |
| | | AGAUGGCCAGGGAGAACCAGACCACUCAAAAGGGGCAGAAGAACUCCAGGGAGCGGAUGAAGCGGAUCGAGGAGGGCAUC |
| | | AAGGAGCUGGGCUCCCAGAUCCUGAAGGAGCACCCCGUGGAGAACACUCAACUGCAGAACGAGAAGCUGUACCUGUACUA |
| | | CCUGCAGAAUGGGCGAGACAUGUACGUGGACCAGGAGCUGGACAUCAACCGGCUCAGCGACUACGACGUGGACCACAUCG |
| | | UUCCCCAGAGCUUCCUGAAGGACGACAGCAUCGACAACAAGGUGCUGACCCGGUCCGACAAGAACCGGGGCAAGUCCGAC |
| | | AACGUUCCCUCAGAGGAAGUCGUGAAGAAGAUGAAGAACUACUGGCGGCAGCUGCUGAACGCCAAGCUGAUCACUCAACG |
| | | GAAGUUCGACAAUCUCACCAAGGCCGAGCGGGGUGGCCUCAGCGAGCUGGACAAGGCCGGGUUCAUCAAGCGGCAGCUGG |
| | | UGGAGACCCGGCAGAUCACCAAGCACGUGGCCCAGAUCCUGGACUCCCGGAUGAACACCAAGUACGACGAGAACGACAAG |
| | | CUGAUCAGGGAAGUCAAGGUGAUCACCCUGAAGUCCAAGCUGGUCAGCGACUUCCGGAAGGACUUCCAGUUCUACAAGGU |
| | | GAGGGAGAUCAACAACUACCACCACGCCCACGACGCCUACCUGAACGCUGUGGUUGGCACCGCACUGAUCAAGAAGUACCC |
| | | CAAGCUGGAGUCCGAGUUCGUGUACGGCGACUACAAGGUGUACGACGUGCGGAAGAUGAUAGCCAAGUCCGAGCAGGAGA |
| | | UCGGCAAGGCCACCGCCAAGUACUUCUUCUACUCCAACAUCAUGAACUUCUUCAAGACAGAGAUCACCCUGGCCAAUGGU |
| | | GAGAUCCGGAAGCGGCCCCUGAUCGAGACCAAUGGUGAGACCGGUGAGAUCGUGUGGGACAAGGGGCGAGACUUCGCCAC |
| | | CGUGCGGAAGGUGCUCAGCAUGCCCCAGGUGAACAUCGUGAAGAAGACAGAAGUCCAGACCGGUGGCUUCUCCAAGGAGA |
| | | GCAUCCUUCCAAAGCGGAACUCCGACAAGCUGAUCGCCCGCAAGAAGGACUGGGACCCCAAGAAGUACGGUGGCUUCGAC |
| | | UCCCCCACCGUGGCCUACUCCGUGCUGGUGGUGGCCAAGGUGGAGAAGGGGAAGUCCAAGAAGCUGAAGUCCGUGAAGGA |
| | | GCUGCUGGGCAUCACCAUCAUGGAGCGGUCCUCCUUCGAGAAGAACCCCAUCGACUUCCUGGAAGCCAAGGGGUACAAGG |
| | | AAGUCAAGAAGGACCUGAUCAUCAAGCUUCCAAAGUACUCCCUGUUCGAGCUGGAGAAUGGGCGGAAGCGGAUGCUGGCC |
| | | UCCGCCGGUGAGCUGCAGAAGGGGAACGAGCUGGCACUUCCCUCAAAGUACGUGAACUUCCUGUACCUGGCCUCCCACUA |
| | | CGAGAAGCUGAAGGGGUCCCCAGAGGACAACGAGCAGAAGCAGCUGUUCGUGGAGCAGCACAAGCACUACCUGGACGAGA |
| | | UCAUCGAGCAGAUCUCCGAGUUCUCCAAGCGGGUGAUCCUGGCCGACGCCAAUCUCGACAAGGUGCUCAGCGCCUACAAC |
| | | AAGCACCGAGACAAGCCCAUCAGGGAGCAGGCCGAGAACAUCAUCCACCUGUUCACCCUGACCAAUCUCGGUGCCCCCGCU |
| | | GCCUUCAAGUACUUCGACACCACCAUCGACCGGAAGCGGUACACCUCGACUAAGGAAGUCCUGGACGCCACCCUGAUCCAC |
| | | CAGAGCAUCACCGGCCUGUACGAGACCCGGAUCGACCUCAGCCAGCUGGGUGGCGACGGUGGUGGCUCCCCCAAGAAGAA |
| | | GCGGAAGGUGUAGCUAGCACCAGCCUCAAGAACACCCGAAUGGAGUCUCUAAGCUACAUAAUACCAACUUACACUUUACA |
| | | AAAUGUUGUCCCCCAAAAUGUAGCCAUUCGUAUCUGCUCCUAAUAAAAAGAAAGUUUCUUCACAUUCUCUCGAGAAAAAA |
| | | AAAAAAAAAAAAAAAAAAAAAAAAAAAAAAAAAAAAAAAAAAAAAAAAAAAAAAAAAAAAAAAAAAAAAAAAAAAAAA |
| | | AAAAAAAAAUCUAG |
| 195 | Cas9 mRNA | GGGAAGCUCAGAAUAAACGCUCAACUUUGGCCGGAUCUGCCACCAUGGACAAGAAGUACUCCAUCGGCCUGGACAUCGGC |
| | transcript | ACCAACUCCGUGGGCUGGGCCGUGAUCACCGACGAGUACAAGGUGCCCUCCAAGAAGUUCAAGGUGCUGGGCAACACCGA |
| | comprising the | CCGGCACUCCAUCAAGAAGAACCUGAUCGGCGCCCUGCUGUUCGACUCCGGCGAGACCGCCGAGGCCACCCGGCUGAAGCG |
| | Cas9 ORF of | GACCGCCCGGCGGCGGUACACCCGGCGGAAGAACCGGAUCUGCUACCUGCAGGAGAUCUUCUCCAACGAGAUGGCCAAGG |
| | SEQ ID No. 3 | UGGACGACUCCUUCUUCCACCGGCUGGAGGAGUCCUUCCUGGUGGAGGAGGACAAGAAGCACGAGCGGCACCCCAUCUUC |
| | and SEQ ID No: | GGCAACAUCGUGGACGAGGUGGCCUACCACGAGAAGUACCCCACCAUCUACCACCUGCGGAAGAAGCUGGUGGACUCCAC |
| | 204 | CGACAAGGCCGACCUGCGGCUGAUCUACCUGGCCCUGGCCCACAUGAUCAAGUUCCGGGGCCACUUCCUGAUCGAGGGCGA |
| | | CCUGAACCCCGACAACUCCGACGUGGACAAGCUGUUCAUCCAGCUGGUGCAGACCUACAACCAGCUGUUCGAGGAGAACCC |
| | | CAUCAACGCCUCCGGCGUGGACGCCAAGGCCAUCCUGUCCGCCCGGCUGUCCAAGUCCCGGCGGCUGGAGAACCUGAUCGC |
| | | CCAGCUGCCCGGCGAGAAGAAGAACGGCCUGUUCGGCAACCUGAUCGCCCUGUCCCUGGGCCUGACCCCCAACUUCAAGUC |
| | | CAACUUCGACCUGGCCGAGGACGCCAAGCUGCAGCUGUCCAAGGACACCUACGACGACGACCUGGACAACCUGCUGGCCCA |
| | | GAUCGGCGACCAGUACGCCGACCUGUUCCUGGCCGCCAAGAACCUGUCCGACGCCAUCCUGCUGUCCGACAUCCUGCGGGU |
| | | GAACACCGAGAUCACCAAGGCCCCCCUGUCCGCCUCCAUGAUCAAGCGGUACGACGAGCACCACCAGGACCUGACCCUGCU |
| | | GAAGGCCCUGGUGCGGCAGCAGCUGCCCGAGAAGUACAAGGAGAUCUUCUUCGACCAGUCCAAGAACGGCUACGCCGGCU |
| | | ACAUCGACGGCGGCGCCUCCCAGGAGGAGUUCUACAAGUUCAUCAAGCCCAUCCUGGAGAAGAUGGACGGCACCGAGGAG |
| | | CUGCUGGUGAAGCUGAACCGGGAGGACCUGCUGCGGAAGCAGCGGACCUUCGACAACGGCUCCAUCCCCCACCAGAUCCAC |
| | | CUGGGCGAGCUGCACGCCAUCCUGCGGCGGCAGGAGGACUUCUACCCCUUCCUGAAGGACAACCGGGAGAAGAUCGAGAA |
| | | GAUCCUGACCUUCCGGAUCCCCUACUACGUGGGCCCCCUGGCCCGGGGCAACUCCCGGUUCGCCUGGAUGACCCGGAAGUC |
| | | CGAGGAGACCAUCACCCCCUGGAACUUCGAGGAGGUGGUGGACAAGGGCGCCUCCGCCCAGUCCUUCAUCGAGCGGAUGA |
| | | CCAACUUCGACAAGAACCUGCCCAACGAGAAGGUGCUGCCCAAGCACUCCCUGCUGUACGAGUACUUCACCGUGUACAACG |
| | | AGCUGACCAAGGUGAAGUACGUGACCGAGGGCAUGCGGAAGCCCGCCUUCCUGUCCGGCGAGCAGAAGAAGGCCAUCGUG |
| | | GACCUGCUGUUCAAGACCAACCGGAAGGUGACCGUGAAGCAGCUGAAGGAGGACUACUUCAAGAAGAUCGAGUGCUUCGA |
| | | CUCCGUGGAGAUCUCCGGCGUGGAGGACCGGUUCAACGCCUCCCUGGGCACCUACCACGACCUGCUGAAGAUCAUCAAGG |
| | | ACAAGGACUUCCUGGACAACGAGGAGAACGAGGACAUCCUGGAGGACAUCGUGCUGACCCUGACCCUGUUCGAGGACCGG |
| | | GAGAUGAUCGAGGAGCGGCUGAAGACCUACGCCCACCUGUUCGACGACAAGGUGAUGAAGCAGCUGAAGCGGCGGCGGUA |
| | | CACCGGCUGGGGCCGGCUGUCCCGGAAGCUGAUCAACGGCAUCCGGGACAAGCAGUCCGGCAAGACCAUCCUGGACUUCCU |
| | | GAAGUCCGACGGCUUCGCCAACCGGAACUUCAUGCAGCUGAUCCACGACGACUCCCUGACCUUCAAGGAGGACAUCCAGA |
| | | AGGCCCAGGUGUCCGGCCAGGGCGACUCCCUGCACGAGCACAUCGCCAACCUGGCCGGCUCCCCCGCCAUCAAGAAGGGCA |
| | | UCCUGCAGACCGUGAAGGUGGUGGACGAGCUGGUGAAGGUGAUGGGCCGGCACAAGCCCGAGAACAUCGUGAUCGAGAUG |
| | | GCCCGGGAGAACCAGACCACCCAGAAGGGCCAGAAGAACUCCCGGGAGCGGAUGAAGCGGAUCGAGGAGGGCAUCAAGGA |
| | | GCUGGGCUCCCAGAUCCUGAAGGAGCACCCCGUGGAGAACACCCAGCUGCAGAACGAGAAGCUGUACCUGUACUACCUGC |
| | | AGAACGGCCGGGACAUGUACGUGGACCAGGAGCUGGACAUCAACCGGCUGUCCGACUACGACGUGGACCACAUCGUGCCC |
| | | CAGUCCUUCCUGAAGGACGACUCCAUCGACAACAAGGUGCUGACCCGGUCCGACAAGAACCGGGGCAAGUCCGACAACGU |
| | | GCCCUCCGAGGAGGUGGUGAAGAAGAUGAAGAACUACUGGCGGCAGCUGCUGAACGCCAAGCUGAUCACCCAGCGGAAGU |
| | | UCGACAACCUGACCAAGGCCGAGCGGGGCGGCCUGUCCGAGCUGGACAAGGCCGGCUUCAUCAAGCGGCAGCUGGUGGAG |
| | | ACCCGGCAGAUCACCAAGCACGUGGCCCAGAUCCUGGACUCCCGGAUGAACACCAAGUACGACGAGAACGACAAGCUGAU |
| | | CCGGGAGGUGAAGGUGAUCACCCUGAAGUCCAAGCUGGUGUCCGACUUCCGGAAGGACUUCCAGUUCUACAAGGUGCGGG |
| | | AGAUCAACAACUACCACCACGCCCACGACGCCUACCUGAACGCCGUGGUGGGCACCGCCCUGAUCAAGAAGUACCCCAAGC |
| | | UGGAGUCCGAGUUCGUGUACGGCGACUACAAGGUGUACGACGUGCGGAAGAUGAUCGCCAAGUCCGAGCAGGAGAUCGGC |
| | | AAGGCCACCGCCAAGUACUUCUUCUACUCCAACAUCAUGAACUUCUUCAAGACCGAGAUCACCCUGGCCAACGGCGAGAUC |
| | | CGGAAGCGGCCCCUGAUCGAGACCAACGGCGAGACCGGCGAGAUCGUGUGGGACAAGGGCCGGGACUUCGCCACCGUGCG |
| | | GAAGGUGCUGUCCAUGCCCCAGGUGAACAUCGUGAAGAAGACCGAGGUGCAGACCGGCGGCUUCUCCAAGGAGUCCAUCC |
| | | UGCCCAAGCGGAACUCCGACAAGCUGAUCGCCCGGAAGAAGGACUGGGACCCCAAGAAGUACGGCGGCUUCGACUCCCCCA |
| | | CCGUGGCCUACUCCGUGCUGGUGGUGGCCAAGGUGGAGAAGGGCAAGUCCAAGAAGCUGAAGUCCGUGAAGGAGCUGCUG |
| | | GGCAUCACCAUCAUGGAGCGGUCCUCCUUCGAGAAGAACCCCAUCGACUUCCUGGAGGCCAAGGGCUACAAGGAGGUGAA |
| | | GAAGGACCUGAUCAUCAAGCUGCCCAAGUACUCCCUGUUCGAGCUGGAGAACGGCCGGAAGCGGAUGCUGGCCUCCGCCG |
| | | GCGAGCUGCAGAAGGGCAACGAGCUGGCCCUGCCCUCCAAGUACGUGAACUUCCUGUACCUGGCCUCCCACUACGAGAAGC |
| | | UGAAGGGCUCCCCCGAGGACAACGAGCAGAAGCAGCUGUUCGUGGAGCAGCACAAGCACUACCUGGACGAGAUCAUCGAG |
| | | CAGAUCUCCGAGUUCUCCAAGCGGGUGAUCCUGGCCGACGCCAACCUGGACAAGGUGCUGUCCGCCUACAACAAGCACCGG |
| | | GACAAGCCCAUCCGGGAGCAGGCCGAGAACAUCAUCCACCUGUUCACCCUGACCAACCUGGGCGCCCCCGCCGCCUUCAAG |
| | | UACUUCGACACCACCAUCGACCGGAAGCGGUACACCUCCACCAAGGAGGUGCUGGACGCCACCCUGAUCCACCAGUCCAUC |
| | | ACCGGCCUGUACGAGACCCGGAUCGACCUGUCCCAGCUGGGCGGCGACGGCGGCGGCUCCCCCAAGAAGAAGCGGAAGGU |
| | | GUGACUAGCACCAGCCUCAAGAACACCCGAAUGGAGUCUCUAAGCUACAUAAUACCAACUUACACUUUACAAAAUGUUGU |
| | | CCCCCAAAAUGUAGCCAUUCGUAUCUGCUCCUAAUAAAAAGAAAGUUUCUUCACAUUCUACCAGCCUCAAGAACACCCGA |
| | | AUGGAGUCUCUAAGCUACAUAAUACCAACUUACACUUUACAAAAUGUUGUCCCCCAAAAUGUAGCCAUUCGUAUCUGCUC |
| | | CUAAUAAAAAGAAAGUUUCUUCACAUUCUCUCGAGAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAUCUAG |
| 196 | Cas9 mRNA | GGGAAGCUCAGAAUAAACGCUCAACUUUGGCCGGAUCUGCCACCAUGGACAAGAAGUACUCCAUCGGCCUGGACAUCGGC |
| | transcript | ACCAACUCCGUGGGCUGGGCCGUGAUCACCGACGAGUACAAGGUGCCCUCCAAGAAGUUCAAGGUGCUGGGCAACACCGA |
| | comprising the | CCGGCACUCCAUCAAGAAGAACCUGAUCGGCGCCCUGCUGUUCGACUCCGGCGAGACCGCCGAGGCCACCCGGCUGAAGCG |
| | Cas9 ORF of | GACCGCCCGGCGGCGGUACACCCGGCGGAAGAACCGGAUCUGCUACCUGCAGGAGAUCUUCUCCAACGAGAUGGCCAAGG |
| | SEQ ID No. 3 | UGGACGACUCCUUCUUCCACCGGCUGGAGGAGUCCUUCCUGGUGGAGGAGGACAAGAAGCACGAGCGGCACCCCAUCUUC |
| | and SEQ ID No: | GGCAACAUCGUGGACGAGGUGGCCUACCACGAGAAGUACCCCACCAUCUACCACCUGCGGAAGAAGCUGGUGGACUCCAC |
| | 202 | CGACAAGGCCGACCUGCGGCUGAUCUACCUGGCCCUGGCCCACAUGAUCAAGUUCCGGGGCCACUUCCUGAUCGAGGGCGA |
| | | CCUGAACCCCGACAACUCCGACGUGGACAAGCUGUUCAUCCAGCUGGUGCAGACCUACAACCAGCUGUUCGAGGAGAACCC |
| | | CAUCAACGCCUCCGGCGUGGACGCCAAGGCCAUCCUGUCCGCCCGGCUGUCCAAGUCCCGGCGGCUGGAGAACCUGAUCGC |
| | | CCAGCUGCCCGGCGAGAAGAAGAACGGCCUGUUCGGCAACCUGAUCGCCCUGUCCCUGGGCCUGACCCCCAACUUCAAGUC |
| | | CAACUUCGACCUGGCCGAGGACGCCAAGCUGCAGCUGUCCAAGGACACCUACGACGACGACCUGGACAACCUGCUGGCCCA |
| | | GAUCGGCGACCAGUACGCCGACCUGUUCCUGGCCGCCAAGAACCUGUCCGACGCCAUCCUGCUGUCCGACAUCCUGCGGGU |
| | | GAACACCGAGAUCACCAAGGCCCCCCUGUCCGCCUCCAUGAUCAAGCGGUACGACGAGCACCACCAGGACCUGACCCUGCU |
| | | GAAGGCCCUGGUGCGGCAGCAGCUGCCCGAGAAGUACAAGGAGAUCUUCUUCGACCAGUCCAAGAACGGCUACGCCGGCU |
| | | ACAUCGACGGCGGCGCCUCCCAGGAGGAGUUCUACAAGUUCAUCAAGCCCAUCCUGGAGAAGAUGGACGGCACCGAGGAG |
| | | CUGCUGGUGAAGCUGAACCGGGAGGACCUGCUGCGGAAGCAGCGGACCUUCGACAACGGCUCCAUCCCCCACCAGAUCCAC |
| | | CUGGGCGAGCUGCACGCCAUCCUGCGGCGGCAGGAGGACUUCUACCCCUUCCUGAAGGACAACCGGGAGAAGAUCGAGAA |
| | | GAUCCUGACCUUCCGGAUCCCCUACUACGUGGGCCCCCUGGCCCGGGGCAACUCCCGGUUCGCCUGGAUGACCCGGAAGUC |
| | | CGAGGAGACCAUCACCCCCUGGAACUUCGAGGAGGUGGUGGACAAGGGCGCCUCCGCCCAGUCCUUCAUCGAGCGGAUGA |
| | | CCAACUUCGACAAGAACCUGCCCAACGAGAAGGUGCUGCCCAAGCACUCCCUGCUGUACGAGUACUUCACCGUGUACAACG |
| | | AGCUGACCAAGGUGAAGUACGUGACCGAGGGCAUGCGGAAGCCCGCCUUCCUGUCCGGCGAGCAGAAGAAGGCCAUCGUG |
| | | GACCUGCUGUUCAAGACCAACCGGAAGGUGACCGUGAAGCAGCUGAAGGAGGACUACUUCAAGAAGAUCGAGUGCUUCGA |
| | | CUCCGUGGAGAUCUCCGGCGUGGAGGACCGGUUCAACGCCUCCCUGGGCACCUACCACGACCUGCUGAAGAUCAUCAAGG |
| | | ACAAGGACUUCCUGGACAACGAGGAGAACGAGGACAUCCUGGAGGACAUCGUGCUGACCCUGACCCUGUUCGAGGACCGG |
| | | GAGAUGAUCGAGGAGCGGCUGAAGACCUACGCCCACCUGUUCGACGACAAGGUGAUGAAGCAGCUGAAGCGGCGGCGGUA |
| | | CACCGGCUGGGGCCGGCUGUCCCGGAAGCUGAUCAACGGCAUCCGGGACAAGCAGUCCGGCAAGACCAUCCUGGACUUCCU |
| | | GAAGUCCGACGGCUUCGCCAACCGGAACUUCAUGCAGCUGAUCCACGACGACUCCCUGACCUUCAAGGAGGACAUCCAGA |
| | | AGGCCCAGGUGUCCGGCCAGGGCGACUCCCUGCACGAGCACAUCGCCAACCUGGCCGGCUCCCCCGCCAUCAAGAAGGGCA |
| | | UCCUGCAGACCGUGAAGGUGGUGGACGAGCUGGUGAAGGUGAUGGGCCGGCACAAGCCCGAGAACAUCGUGAUCGAGAUG |
| | | GCCCGGGAGAACCAGACCACCCAGAAGGGCCAGAAGAACUCCCGGGAGCGGAUGAAGCGGAUCGAGGAGGGCAUCAAGGA |
| | | GCUGGGCUCCCAGAUCCUGAAGGAGCACCCCGUGGAGAACACCCAGCUGCAGAACGAGAAGCUGUACCUGUACUACCUGC |
| | | AGAACGGCCGGGACAUGUACGUGGACCAGGAGCUGGACAUCAACCGGCUGUCCGACUACGACGUGGACCACAUCGUGCCC |
| | | CAGUCCUUCCUGAAGGACGACUCCAUCGACAACAAGGUGCUGACCCGGUCCGACAAGAACCGGGGCAAGUCCGACAACGU |
| | | GCCCUCCGAGGAGGUGGUGAAGAAGAUGAAGAACUACUGGCGGCAGCUGCUGAACGCCAAGCUGAUCACCCAGCGGAAGU |
| | | UCGACAACCUGACCAAGGCCGAGCGGGGCGGCCUGUCCGAGCUGGACAAGGCCGGCUUCAUCAAGCGGCAGCUGGUGGAG |
| | | ACCCGGCAGAUCACCAAGCACGUGGCCCAGAUCCUGGACUCCCGGAUGAACACCAAGUACGACGAGAACGACAAGCUGAU |
| | | CCGGGAGGUGAAGGUGAUCACCCUGAAGUCCAAGCUGGUGUCCGACUUCCGGAAGGACUUCCAGUUCUACAAGGUGCGGG |
| | | AGAUCAACAACUACCACCACGCCCACGACGCCUACCUGAACGCCGUGGUGGGCACCGCCCUGAUCAAGAAGUACCCCAAGC |
| | | UGGAGUCCGAGUUCGUGUACGGCGACUACAAGGUGUACGACGUGCGGAAGAUGAUCGCCAAGUCCGAGCAGGAGAUCGGC |
| | | AAGGCCACCGCCAAGUACUUCUUCUACUCCAACAUCAUGAACUUCUUCAAGACCGAGAUCACCCUGGCCAACGGCGAGAUC |
| | | CGGAAGCGGCCCCUGAUCGAGACCAACGGCGAGACCGGCGAGAUCGUGUGGGACAAGGGCCGGGACUUCGCCACCGUGCG |
| | | GAAGGUGCUGUCCAUGCCCCAGGUGAACAUCGUGAAGAAGACCGAGGUGCAGACCGGCGGCUUCUCCAAGGAGUCCAUCC |
| | | UGCCCAAGCGGAACUCCGACAAGCUGAUCGCCCGGAAGAAGGACUGGGACCCCAAGAAGUACGGCGGCUUCGACUCCCCCA |
| | | CCGUGGCCUACUCCGUGCUGGUGGUGGCCAAGGUGGAGAAGGGCAAGUCCAAGAAGCUGAAGUCCGUGAAGGAGCUGCUG |
| | | GGCAUCACCAUCAUGGAGCGGUCCUCCUUCGAGAAGAACCCCAUCGACUUCCUGGAGGCCAAGGGCUACAAGGAGGUGAA |
| | | GAAGGACCUGAUCAUCAAGCUGCCCAAGUACUCCCUGUUCGAGCUGGAGAACGGCCGGAAGCGGAUGCUGGCCUCCGCCG |
| | | GCGAGCUGCAGAAGGGCAACGAGCUGGCCCUGCCCUCCAAGUACGUGAACUUCCUGUACCUGGCCUCCCACUACGAGAAGC |
| | | UGAAGGGCUCCCCCGAGGACAACGAGCAGAAGCAGCUGUUCGUGGAGCAGCACAAGCACUACCUGGACGAGAUCAUCGAG |
| | | CAGAUCUCCGAGUUCUCCAAGCGGGUGAUCCUGGCCGACGCCAACCUGGACAAGGUGCUGUCCGCCUACAACAAGCACCGG |
| | | GACAAGCCCAUCCGGGAGCAGGCCGAGAACAUCAUCCACCUGUUCACCCUGACCAACCUGGGCGCCCCCGCCGCCUUCAAG |
| | | UACUUCGACACCACCAUCGACCGGAAGCGGUACACCUCCACCAAGGAGGUGCUGGACGCCACCCUGAUCCACCAGUCCAUC |
| | | ACCGGCCUGUACGAGACCCGGAUCGACCUGUCCCAGCUGGGCGGCGACGGCGGCGGCUCCCCCAAGAAGAAGCGGAAGGU |
| | | GUGACUGGUACUGCAUGCACGCAAUGCUAGCUGCCCCUUUCCCGUCCUGGGUACCCCGAGUCUCCCCCGACCUCGGGUCCC |
| | | AGGUAUGCUCCCACCUCCACCUGCCCCACUCACCACCUCUGCUAGUUCCAGACACCUCCCAAGCACGCAGCAAUGCAGCUC |
| | | AAAACGCUUAGCCUAGCCACACCCCCACGGGAAACAGCAGUGAUUAACCUUUAGCAAUAAACGAAAGUUUAACUAAGCUA |
| | | UACUAACCCCAGGGUUGGUCAAUUUCGUGCCAGCCACACCCUCGAGAAAAAAAAAAAAAAAAAAAAAAAAAAAAAAAAAA |
| | | AAAAAAAAAAAAAAAAAAAAAAAAAAAAAAAAAAAAAAAAAAAAAAAAAAAAAAAAAAAUCUAG |
| 197 | Cas9 mRNA | GGGAAGCUCAGAAUAAACGCUCAACUUUGGCCGGAUCUGCCACCAUGGACAAGAAGUACUCCAUCGGCCUGGACAUCGGC |
| | transcript | ACCAACUCCGUGGGCUGGGCCGUGAUCACCGACGAGUACAAGGUGCCCUCCAAGAAGUUCAAGGUGCUGGGCAACACCGA |
| | comprising the | CCGGCACUCCAUCAAGAAGAACCUGAUCGGCGCCCUGCUGUUCGACUCCGGCGAGACCGCCGAGGCCACCCGGCUGAAGCG |
| | Cas9 ORF of | GACCGCCCGGCGGCGGUACACCCGGCGGAAGAACCGGAUCUGCUACCUGCAGGAGAUCUUCUCCAACGAGAUGGCCAAGG |
| | SEQ ID No. 3 | UGGACGACUCCUUCUUCCACCGGCUGGAGGAGUCCUUCCUGGUGGAGGAGGACAAGAAGCACGAGCGGCACCCCAUCUUC |
| | and SEQ ID No: | GGCAACAUCGUGGACGAGGUGGCCUACCACGAGAAGUACCCCACCAUCUACCACCUGCGGAAGAAGCUGGUGGACUCCAC |
| | 203 | CGACAAGGCCGACCUGCGGCUGAUCUACCUGGCCCUGGCCCACAUGAUCAAGUUCCGGGGCCACUUCCUGAUCGAGGGCGA |
| | | CCUGAACCCCGACAACUCCGACGUGGACAAGCUGUUCAUCCAGCUGGUGCAGACCUACAACCAGCUGUUCGAGGAGAACCC |
| | | CAUCAACGCCUCCGGCGUGGACGCCAAGGCCAUCCUGUCCGCCCGGCUGUCCAAGUCCCGGCGGCUGGAGAACCUGAUCGC |
| | | CCAGCUGCCCGGCGAGAAGAAGAACGGCCUGUUCGGCAACCUGAUCGCCCUGUCCCUGGGCCUGACCCCCAACUUCAAGUC |
| | | CAACUUCGACCUGGCCGAGGACGCCAAGCUGCAGCUGUCCAAGGACACCUACGACGACGACCUGGACAACCUGCUGGCCCA |
| | | GAUCGGCGACCAGUACGCCGACCUGUUCCUGGCCGCCAAGAACCUGUCCGACGCCAUCCUGCUGUCCGACAUCCUGCGGGU |
| | | GAACACCGAGAUCACCAAGGCCCCCCUGUCCGCCUCCAUGAUCAAGCGGUACGACGAGCACCACCAGGACCUGACCCUGCU |
| | | GAAGGCCCUGGUGCGGCAGCAGCUGCCCGAGAAGUACAAGGAGAUCUUCUUCGACCAGUCCAAGAACGGCUACGCCGGCU |
| | | ACAUCGACGGCGGCGCCUCCCAGGAGGAGUUCUACAAGUUCAUCAAGCCCAUCCUGGAGAAGAUGGACGGCACCGAGGAG |
| | | CUGCUGGUGAAGCUGAACCGGGAGGACCUGCUGCGGAAGCAGCGGACCUUCGACAACGGCUCCAUCCCCCACCAGAUCCAC |
| | | CUGGGCGAGCUGCACGCCAUCCUGCGGCGGCAGGAGGACUUCUACCCCUUCCUGAAGGACAACCGGGAGAAGAUCGAGAA |
| | | GAUCCUGACCUUCCGGAUCCCCUACUACGUGGGCCCCCUGGCCCGGGGCAACUCCCGGUUCGCCUGGAUGACCCGGAAGUC |
| | | CGAGGAGACCAUCACCCCCUGGAACUUCGAGGAGGUGGUGGACAAGGGCGCCUCCGCCCAGUCCUUCAUCGAGCGGAUGA |
| | | CCAACUUCGACAAGAACCUGCCCAACGAGAAGGUGCUGCCCAAGCACUCCCUGCUGUACGAGUACUUCACCGUGUACAACG |
| | | AGCUGACCAAGGUGAAGUACGUGACCGAGGGCAUGCGGAAGCCCGCCUUCCUGUCCGGCGAGCAGAAGAAGGCCAUCGUG |
| | | GACCUGCUGUUCAAGACCAACCGGAAGGUGACCGUGAAGCAGCUGAAGGAGGACUACUUCAAGAAGAUCGAGUGCUUCGA |
| | | CUCCGUGGAGAUCUCCGGCGUGGAGGACCGGUUCAACGCCUCCCUGGGCACCUACCACGACCUGCUGAAGAUCAUCAAGG |
| | | ACAAGGACUUCCUGGACAACGAGGAGAACGAGGACAUCCUGGAGGACAUCGUGCUGACCCUGACCCUGUUCGAGGACCGG |
| | | GAGAUGAUCGAGGAGCGGCUGAAGACCUACGCCCACCUGUUCGACGACAAGGUGAUGAAGCAGCUGAAGCGGCGGCGGUA |
| | | CACCGGCUGGGGCCGGCUGUCCCGGAAGCUGAUCAACGGCAUCCGGGACAAGCAGUCCGGCAAGACCAUCCUGGACUUCCU |
| | | GAAGUCCGACGGCUUCGCCAACCGGAACUUCAUGCAGCUGAUCCACGACGACUCCCUGACCUUCAAGGAGGACAUCCAGA |
| | | AGGCCCAGGUGUCCGGCCAGGGCGACUCCCUGCACGAGCACAUCGCCAACCUGGCCGGCUCCCCCGCCAUCAAGAAGGGCA |
| | | UCCUGCAGACCGUGAAGGUGGUGGACGAGCUGGUGAAGGUGAUGGGCCGGCACAAGCCCGAGAACAUCGUGAUCGAGAUG |
| | | GCCCGGGAGAACCAGACCACCCAGAAGGGCCAGAAGAACUCCCGGGAGCGGAUGAAGCGGAUCGAGGAGGGCAUCAAGGA |
| | | GCUGGGCUCCCAGAUCCUGAAGGAGCACCCCGUGGAGAACACCCAGCUGCAGAACGAGAAGCUGUACCUGUACUACCUGC |
| | | AGAACGGCCGGGACAUGUACGUGGACCAGGAGCUGGACAUCAACCGGCUGUCCGACUACGACGUGGACCACAUCGUGCCC |
| | | CAGUCCUUCCUGAAGGACGACUCCAUCGACAACAAGGUGCUGACCCGGUCCGACAAGAACCGGGGCAAGUCCGACAACGU |
| | | GCCCUCCGAGGAGGUGGUGAAGAAGAUGAAGAACUACUGGCGGCAGCUGCUGAACGCCAAGCUGAUCACCCAGCGGAAGU |
| | | UCGACAACCUGACCAAGGCCGAGCGGGGCGGCCUGUCCGAGCUGGACAAGGCCGGCUUCAUCAAGCGGCAGCUGGUGGAG |
| | | ACCCGGCAGAUCACCAAGCACGUGGCCCAGAUCCUGGACUCCCGGAUGAACACCAAGUACGACGAGAACGACAAGCUGAU |
| | | CCGGGAGGUGAAGGUGAUCACCCUGAAGUCCAAGCUGGUGUCCGACUUCCGGAAGGACUUCCAGUUCUACAAGGUGCGGG |
| | | AGAUCAACAACUACCACCACGCCCACGACGCCUACCUGAACGCCGUGGUGGGCACCGCCCUGAUCAAGAAGUACCCCAAGC |
| | | UGGAGUCCGAGUUCGUGUACGGCGACUACAAGGUGUACGACGUGCGGAAGAUGAUCGCCAAGUCCGAGCAGGAGAUCGGC |
| | | AAGGCCACCGCCAAGUACUUCUUCUACUCCAACAUCAUGAACUUCUUCAAGACCGAGAUCACCCUGGCCAACGGCGAGAUC |
| | | CGGAAGCGGCCCCUGAUCGAGACCAACGGCGAGACCGGCGAGAUCGUGUGGGACAAGGGCCGGGACUUCGCCACCGUGCG |
| | | GAAGGUGCUGUCCAUGCCCCAGGUGAACAUCGUGAAGAAGACCGAGGUGCAGACCGGCGGCUUCUCCAAGGAGUCCAUCC |
| | | UGCCCAAGCGGAACUCCGACAAGCUGAUCGCCCGGAAGAAGGACUGGGACCCCAAGAAGUACGGCGGCUUCGACUCCCCCA |
| | | CCGUGGCCUACUCCGUGCUGGUGGUGGCCAAGGUGGAGAAGGGCAAGUCCAAGAAGCUGAAGUCCGUGAAGGAGCUGCUG |
| | | GGCAUCACCAUCAUGGAGCGGUCCUCCUUCGAGAAGAACCCCAUCGACUUCCUGGAGGCCAAGGGCUACAAGGAGGUGAA |
| | | GAAGGACCUGAUCAUCAAGCUGCCCAAGUACUCCCUGUUCGAGCUGGAGAACGGCCGGAAGCGGAUGCUGGCCUCCGCCG |
| | | GCGAGCUGCAGAAGGGCAACGAGCUGGCCCUGCCCUCCAAGUACGUGAACUUCCUGUACCUGGCCUCCCACUACGAGAAGC |
| | | UGAAGGGCUCCCCCGAGGACAACGAGCAGAAGCAGCUGUUCGUGGAGCAGCACAAGCACUACCUGGACGAGAUCAUCGAG |
| | | CAGAUCUCCGAGUUCUCCAAGCGGGUGAUCCUGGCCGACGCCAACCUGGACAAGGUGCUGUCCGCCUACAACAAGCACCGG |
| | | GACAAGCCCAUCCGGGAGCAGGCCGAGAACAUCAUCCACCUGUUCACCCUGACCAACCUGGGCGCCCCCGCCGCCUUCAAG |
| | | UACUUCGACACCACCAUCGACCGGAAGCGGUACACCUCCACCAAGGAGGUGCUGGACGCCACCCUGAUCCACCAGUCCAUC |
| | | ACCGGCCUGUACGAGACCCGGAUCGACCUGUCCCAGCUGGGCGGCGACGGCGGCGGCUCCCCCAAGAAGAAGCGGAAGGU |
| | | GUGACAAGCACGCAGCAAUGCAGCUCAAAACGCUUAGCCUAGCCACACCCCCACGGGAAACAGCAGUGAUUAACCUUUAG |
| | | CAAUAAACGAAAGUUUAACUAAGCUAUACUAACCCCAGGGUUGGUCAAUUUCGUGCCAGCCACACCCUGGUACUGCAUGC |
| | | ACGCAAUGCUAGCUGCCCCUUUCCCGUCCUGGGUACCCCGAGUCUCCCCCGACCUCGGGUCCCAGGUAUGCUCCCACCUCC |
| | | ACCUGCCCCACUCACCACCUCUGCUAGUUCCAGACACCUCCCUCGAGAAAAAAAAAAAAAAAAAAAAAAAAAAAAAAAAA |
| | | AAAAAAAAAAAAAAAAAAAAAAAAAAAAAAAAAAAAAAAAAAAAAAAAAAAAAAAAAAAAUCUAG |
| 198 | | Not Used |
| 199 | Cas9 mRNA | GGGAAGCUCAGAAUAAACGCUCAACUUUGGCCGGAUCUGCCACCAUGGACAAGAAGUACAGCAUCGGCCUGGACAUCGGC |
| | transcript | ACCAACUCCGUUGGCUGGGCUGUGAUCACCGACGAGUACAAGGUUCCCUCAAAGAAGUUCAAGGUGCUGGGCAACACCGA |
| | comprising SEQ | CCGGCACAGCAUCAAGAAGAAUCUCAUCGGUGCACUGCUGUUCGACUCCGGUGAGACCGCCGAAGCCACCCGGCUGAAGC |
| | ID No: 46 and | GGACCGCCCGCCGGCGGUACACCCGGCGGAAGAACCGGAUCUGCUACCUGCAGGAGAUCUUCUCCAACGAGAUGGCCAAG |
| | SEQ ID No: 204 | GUGGACGACUCCUUCUUCCACCGGCUGGAGGAGUCCUUCCUGGUGGAGGAGGACAAGAAGCACGAGCGGCACCCCAUCUU |
| | | CGGCAACAUCGUGGACGAAGUCGCCUACCACGAGAAGUACCCCACCAUCUACCACCUGCGGAAGAAGCUGGUGGACUCGA |
| | | CUGACAAGGCCGACCUGCGGCUGAUCUACCUGGCACUGGCCCACAUGAUAAAGUUCCGGGGCCACUUCCUGAUCGAGGGC |
| | | GACCUGAACCCUGACAACUCCGACGUGGACAAGCUGUUCAUCCAGCUGGUGCAGACCUACAACCAGCUGUUCGAGGAGAA |
| | | CCCCAUCAACGCCUCCGGCGUGGACGCCAAGGCCAUCCUCAGCGCCCGCCUCAGCAAGUCCCGGCGGCUGGAGAAUCUCAU |
| | | CGCCCAGCUUCCAGGUGAGAAGAAGAAUGGGCUGUUCGGCAAUCUCAUCGCACUCAGCCUGGGCCUGACUCCCAACUUCA |
| | | AGUCCAACUUCGACCUGGCCGAGGACGCCAAGCUGCAGCUCAGCAAGGACACCUACGACGACGACCUGGACAAUCUCCUGG |
| | | CCCAGAUCGGCGACCAGUACGCCGACCUGUUCCUGGCUGCCAAGAAUCUCAGCGACGCCAUCCUGCUCAGCGACAUCCUGC |
| | | GGGUGAACACAGAGAUCACCAAGGCCCCCCUCAGCGCCUCCAUGAUAAAGCGGUACGACGAGCACCACCAGGACCUGACCC |
| | | UGCUGAAGGCACUGGUGCGGCAGCAGCUUCCAGAGAAGUACAAGGAGAUCUUCUUCGACCAGAGCAAGAAUGGGUACGCC |
| | | GGGUACAUCGACGGUGGUGCCUCCCAGGAGGAGUUCUACAAGUUCAUCAAGCCCAUCCUGGAGAAGAUGGACGGCACAGA |
| | | GGAGCUGCUGGUGAAGCUGAACAGGGAGGACCUGCUGCGGAAGCAGCGGACCUUCGACAAUGGGAGCAUCCCCCACCAGA |
| | | UCCACCUGGGUGAGCUGCACGCCAUCCUGCGGCGGCAGGAGGACUUCUACCCCUUCCUGAAGGACAACAGGGAGAAGAUC |
| | | GAGAAGAUCCUGACCUUCCGGAUCCCCUACUACGUUGGCCCCCUGGCCCGCGGCAACUCCCGGUUCGCCUGGAUGACCCGG |
| | | AAGUCCGAGGAGACCAUCACUCCCUGGAACUUCGAGGAAGUCGUGGACAAGGGUGCCUCCGCCCAGAGCUUCAUCGAGCG |
| | | GAUGACCAACUUCGACAAGAAUCUUCCAAACGAGAAGGUGCUUCCAAAGCACUCCCUGCUGUACGAGUACUUCACCGUGU |
| | | ACAACGAGCUGACCAAGGUGAAGUACGUGACAGAGGGCAUGCGGAAGCCCGCCUUCCUCAGCGGUGAGCAGAAGAAGGCC |
| | | AUCGUGGACCUGCUGUUCAAGACCAACCGGAAGGUGACCGUGAAGCAGCUGAAGGAGGACUACUUCAAGAAGAUCGAGUG |
| | | CUUCGACUCCGUGGAGAUCUCCGGCGUGGAGGACCGGUUCAACGCCUCCCUGGGCACCUACCACGACCUGCUGAAGAUCAU |
| | | CAAGGACAAGGACUUCCUGGACAACGAGGAGAACGAGGACAUCCUGGAGGACAUCGUGCUGACCCUGACCCUGUUCGAGG |
| | | ACAGGGAGAUGAUAGAGGAGCGGCUGAAGACCUACGCCCACCUGUUCGACGACAAGGUGAUGAAGCAGCUGAAGCGGCGG |
| | | CGGUACACCGGCUGGGGCCGGCUCAGCCGGAAGCUGAUCAAUGGGAUCCGAGACAAGCAGAGCGGCAAGACCAUCCUGGA |
| | | CUUCCUGAAGUCCGACGGCUUCGCCAACCGGAACUUCAUGCAGCUGAUCCACGACGACUCCCUGACCUUCAAGGAGGACAU |
| | | CCAGAAGGCCCAGGUCAGCGGCCAGGGCGACUCCCUGCACGAGCACAUCGCCAAUCUCGCCGGGUCCCCCGCCAUCAAGAA |
| | | GGGGAUCCUGCAGACCGUGAAGGUGGUGGACGAGCUGGUGAAGGUGAUGGGCCGGCACAAGCCAGAGAACAUCGUGAUCG |
| | | AGAUGGCCAGGGAGAACCAGACCACUCAAAAGGGGCAGAAGAACUCCAGGGAGCGGAUGAAGCGGAUCGAGGAGGGCAUC |
| | | AAGGAGCUGGGCUCCCAGAUCCUGAAGGAGCACCCCGUGGAGAACACUCAACUGCAGAACGAGAAGCUGUACCUGUACUA |
| | | CCUGCAGAAUGGGCGAGACAUGUACGUGGACCAGGAGCUGGACAUCAACCGGCUCAGCGACUACGACGUGGACCACAUCG |
| | | UUCCCCAGAGCUUCCUGAAGGACGACAGCAUCGACAACAAGGUGCUGACCCGGUCCGACAAGAACCGGGGCAAGUCCGAC |
| | | AACGUUCCCUCAGAGGAAGUCGUGAAGAAGAUGAAGAACUACUGGCGGCAGCUGCUGAACGCCAAGCUGAUCACUCAACG |
| | | GAAGUUCGACAAUCUCACCAAGGCCGAGCGGGGUGGCCUCAGCGAGCUGGACAAGGCCGGGUUCAUCAAGCGGCAGCUGG |
| | | UGGAGACCCGGCAGAUCACCAAGCACGUGGCCCAGAUCCUGGACUCCCGGAUGAACACCAAGUACGACGAGAACGACAAG |
| | | CUGAUCAGGGAAGUCAAGGUGAUCACCCUGAAGUCCAAGCUGGUCAGCGACUUCCGGAAGGACUUCCAGUUCUACAAGGU |
| | | GAGGGAGAUCAACAACUACCACCACGCCCACGACGCCUACCUGAACGCUGUGGUUGGCACCGCACUGAUCAAGAAGUACCC |
| | | CAAGCUGGAGUCCGAGUUCGUGUACGGCGACUACAAGGUGUACGACGUGCGGAAGAUGAUAGCCAAGUCCGAGCAGGAGA |
| | | UCGGCAAGGCCACCGCCAAGUACUUCUUCUACUCCAACAUCAUGAACUUCUUCAAGACAGAGAUCACCCUGGCCAAUGGU |
| | | GAGAUCCGGAAGCGGCCCCUGAUCGAGACCAAUGGUGAGACCGGUGAGAUCGUGUGGGACAAGGGGCGAGACUUCGCCAC |
| | | CGUGCGGAAGGUGCUCAGCAUGCCCCAGGUGAACAUCGUGAAGAAGACAGAAGUCCAGACCGGUGGCUUCUCCAAGGAGA |
| | | GCAUCCUUCCAAAGCGGAACUCCGACAAGCUGAUCGCCCGCAAGAAGGACUGGGACCCCAAGAAGUACGGUGGCUUCGAC |
| | | UCCCCCACCGUGGCCUACUCCGUGCUGGUGGUGGCCAAGGUGGAGAAGGGGAAGUCCAAGAAGCUGAAGUCCGUGAAGGA |
| | | GCUGCUGGGCAUCACCAUCAUGGAGCGGUCCUCCUUCGAGAAGAACCCCAUCGACUUCCUGGAAGCCAAGGGGUACAAGG |
| | | AAGUCAAGAAGGACCUGAUCAUCAAGCUUCCAAAGUACUCCCUGUUCGAGCUGGAGAAUGGGCGGAAGCGGAUGCUGGCC |
| | | UCCGCCGGUGAGCUGCAGAAGGGGAACGAGCUGGCACUUCCCUCAAAGUACGUGAACUUCCUGUACCUGGCCUCCCACUA |
| | | CGAGAAGCUGAAGGGGUCCCCAGAGGACAACGAGCAGAAGCAGCUGUUCGUGGAGCAGCACAAGCACUACCUGGACGAGA |
| | | UCAUCGAGCAGAUCUCCGAGUUCUCCAAGCGGGUGAUCCUGGCCGACGCCAAUCUCGACAAGGUGCUCAGCGCCUACAAC |
| | | AAGCACCGAGACAAGCCCAUCAGGGAGCAGGCCGAGAACAUCAUCCACCUGUUCACCCUGACCAAUCUCGGUGCCCCCGCU |
| | | GCCUUCAAGUACUUCGACACCACCAUCGACCGGAAGCGGUACACCUCGACUAAGGAAGUCCUGGACGCCACCCUGAUCCAC |
| | | CAGAGCAUCACCGGCCUGUACGAGACCCGGAUCGACCUCAGCCAGCUGGGUGGCGACGGUGGUGGCUCCCCCAAGAAGAA |
| | | GCGGAAGGUGUAGCUAGCACCAGCCUCAAGAACACCCGAAUGGAGUCUCUAAGCUACAUAAUACCAACUUACACUUUACA |
| | | AAAUGUUGUCCCCCAAAAUGUAGCCAUUCGUAUCUGCUCCUAAUAAAAAGAAAGUUUCUUCACAUUCUACCAGCCUCAAG |
| | | AACACCCGAAUGGAGUCUCUAAGCUACAUAAUACCAACUUACACUUUACAAAAUGUUGUCCCCCAAAAUGUAGCCAUUCG |
| | | UAUCUGCUCCUAAUAAAAAGAAAGUUUCUUCACAUUCUCUCGAGAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAAAAAAAAAAAAAAAAUCUAG |
| 200 | Cas9 mRNA | GGGAAGCUCAGAAUAAACGCUCAACUUUGGCCGGAUCUGCCACCAUGGACAAGAAGUACAGCAUCGGCCUGGACAUCGGC |
| | transcript | ACCAACUCCGUUGGCUGGGCUGUGAUCACCGACGAGUACAAGGUUCCCUCAAAGAAGUUCAAGGUGCUGGGCAACACCGA |
| | comprising SEQ | CCGGCACAGCAUCAAGAAGAAUCUCAUCGGUGCACUGCUGUUCGACUCCGGUGAGACCGCCGAAGCCACCCGGCUGAAGC |
| | ID No: 46 and | GGACCGCCCGCCGGCGGUACACCCGGCGGAAGAACCGGAUCUGCUACCUGCAGGAGAUCUUCUCCAACGAGAUGGCCAAG |
| | SEQ ID No: 202 | GUGGACGACUCCUUCUUCCACCGGCUGGAGGAGUCCUUCCUGGUGGAGGAGGACAAGAAGCACGAGCGGCACCCCAUCUU |
| | | CGGCAACAUCGUGGACGAAGUCGCCUACCACGAGAAGUACCCCACCAUCUACCACCUGCGGAAGAAGCUGGUGGACUCGA |
| | | CUGACAAGGCCGACCUGCGGCUGAUCUACCUGGCACUGGCCCACAUGAUAAAGUUCCGGGGCCACUUCCUGAUCGAGGGC |
| | | GACCUGAACCCUGACAACUCCGACGUGGACAAGCUGUUCAUCCAGCUGGUGCAGACCUACAACCAGCUGUUCGAGGAGAA |
| | | CCCCAUCAACGCCUCCGGCGUGGACGCCAAGGCCAUCCUCAGCGCCCGCCUCAGCAAGUCCCGGCGGCUGGAGAAUCUCAU |
| | | CGCCCAGCUUCCAGGUGAGAAGAAGAAUGGGCUGUUCGGCAAUCUCAUCGCACUCAGCCUGGGCCUGACUCCCAACUUCA |
| | | AGUCCAACUUCGACCUGGCCGAGGACGCCAAGCUGCAGCUCAGCAAGGACACCUACGACGACGACCUGGACAAUCUCCUGG |
| | | CCCAGAUCGGCGACCAGUACGCCGACCUGUUCCUGGCUGCCAAGAAUCUCAGCGACGCCAUCCUGCUCAGCGACAUCCUGC |
| | | GGGUGAACACAGAGAUCACCAAGGCCCCCCUCAGCGCCUCCAUGAUAAAGCGGUACGACGAGCACCACCAGGACCUGACCC |
| | | UGCUGAAGGCACUGGUGCGGCAGCAGCUUCCAGAGAAGUACAAGGAGAUCUUCUUCGACCAGAGCAAGAAUGGGUACGCC |
| | | GGGUACAUCGACGGUGGUGCCUCCCAGGAGGAGUUCUACAAGUUCAUCAAGCCCAUCCUGGAGAAGAUGGACGGCACAGA |
| | | GGAGCUGCUGGUGAAGCUGAACAGGGAGGACCUGCUGCGGAAGCAGCGGACCUUCGACAAUGGGAGCAUCCCCCACCAGA |
| | | UCCACCUGGGUGAGCUGCACGCCAUCCUGCGGCGGCAGGAGGACUUCUACCCCUUCCUGAAGGACAACAGGGAGAAGAUC |
| | | GAGAAGAUCCUGACCUUCCGGAUCCCCUACUACGUUGGCCCCCUGGCCCGCGGCAACUCCCGGUUCGCCUGGAUGACCCGG |
| | | AAGUCCGAGGAGACCAUCACUCCCUGGAACUUCGAGGAAGUCGUGGACAAGGGUGCCUCCGCCCAGAGCUUCAUCGAGCG |
| | | GAUGACCAACUUCGACAAGAAUCUUCCAAACGAGAAGGUGCUUCCAAAGCACUCCCUGCUGUACGAGUACUUCACCGUGU |
| | | ACAACGAGCUGACCAAGGUGAAGUACGUGACAGAGGGCAUGCGGAAGCCCGCCUUCCUCAGCGGUGAGCAGAAGAAGGCC |
| | | AUCGUGGACCUGCUGUUCAAGACCAACCGGAAGGUGACCGUGAAGCAGCUGAAGGAGGACUACUUCAAGAAGAUCGAGUG |
| | | CUUCGACUCCGUGGAGAUCUCCGGCGUGGAGGACCGGUUCAACGCCUCCCUGGGCACCUACCACGACCUGCUGAAGAUCAU |
| | | CAAGGACAAGGACUUCCUGGACAACGAGGAGAACGAGGACAUCCUGGAGGACAUCGUGCUGACCCUGACCCUGUUCGAGG |
| | | ACAGGGAGAUGAUAGAGGAGCGGCUGAAGACCUACGCCCACCUGUUCGACGACAAGGUGAUGAAGCAGCUGAAGCGGCGG |
| | | CGGUACACCGGCUGGGGCCGGCUCAGCCGGAAGCUGAUCAAUGGGAUCCGAGACAAGCAGAGCGGCAAGACCAUCCUGGA |
| | | CUUCCUGAAGUCCGACGGCUUCGCCAACCGGAACUUCAUGCAGCUGAUCCACGACGACUCCCUGACCUUCAAGGAGGACAU |
| | | CCAGAAGGCCCAGGUCAGCGGCCAGGGCGACUCCCUGCACGAGCACAUCGCCAAUCUCGCCGGGUCCCCCGCCAUCAAGAA |
| | | GGGGAUCCUGCAGACCGUGAAGGUGGUGGACGAGCUGGUGAAGGUGAUGGGCCGGCACAAGCCAGAGAACAUCGUGAUCG |
| | | AGAUGGCCAGGGAGAACCAGACCACUCAAAAGGGGCAGAAGAACUCCAGGGAGCGGAUGAAGCGGAUCGAGGAGGGCAUC |
| | | AAGGAGCUGGGCUCCCAGAUCCUGAAGGAGCACCCCGUGGAGAACACUCAACUGCAGAACGAGAAGCUGUACCUGUACUA |
| | | CCUGCAGAAUGGGCGAGACAUGUACGUGGACCAGGAGCUGGACAUCAACCGGCUCAGCGACUACGACGUGGACCACAUCG |
| | | UUCCCCAGAGCUUCCUGAAGGACGACAGCAUCGACAACAAGGUGCUGACCCGGUCCGACAAGAACCGGGGCAAGUCCGAC |
| | | AACGUUCCCUCAGAGGAAGUCGUGAAGAAGAUGAAGAACUACUGGCGGCAGCUGCUGAACGCCAAGCUGAUCACUCAACG |
| | | GAAGUUCGACAAUCUCACCAAGGCCGAGCGGGGUGGCCUCAGCGAGCUGGACAAGGCCGGGUUCAUCAAGCGGCAGCUGG |
| | | UGGAGACCCGGCAGAUCACCAAGCACGUGGCCCAGAUCCUGGACUCCCGGAUGAACACCAAGUACGACGAGAACGACAAG |
| | | CUGAUCAGGGAAGUCAAGGUGAUCACCCUGAAGUCCAAGCUGGUCAGCGACUUCCGGAAGGACUUCCAGUUCUACAAGGU |
| | | GAGGGAGAUCAACAACUACCACCACGCCCACGACGCCUACCUGAACGCUGUGGUUGGCACCGCACUGAUCAAGAAGUACCC |
| | | CAAGCUGGAGUCCGAGUUCGUGUACGGCGACUACAAGGUGUACGACGUGCGGAAGAUGAUAGCCAAGUCCGAGCAGGAGA |
| | | UCGGCAAGGCCACCGCCAAGUACUUCUUCUACUCCAACAUCAUGAACUUCUUCAAGACAGAGAUCACCCUGGCCAAUGGU |
| | | GAGAUCCGGAAGCGGCCCCUGAUCGAGACCAAUGGUGAGACCGGUGAGAUCGUGUGGGACAAGGGGCGAGACUUCGCCAC |
| | | CGUGCGGAAGGUGCUCAGCAUGCCCCAGGUGAACAUCGUGAAGAAGACAGAAGUCCAGACCGGUGGCUUCUCCAAGGAGA |
| | | GCAUCCUUCCAAAGCGGAACUCCGACAAGCUGAUCGCCCGCAAGAAGGACUGGGACCCCAAGAAGUACGGUGGCUUCGAC |
| | | UCCCCCACCGUGGCCUACUCCGUGCUGGUGGUGGCCAAGGUGGAGAAGGGGAAGUCCAAGAAGCUGAAGUCCGUGAAGGA |
| | | GCUGCUGGGCAUCACCAUCAUGGAGCGGUCCUCCUUCGAGAAGAACCCCAUCGACUUCCUGGAAGCCAAGGGGUACAAGG |
| | | AAGUCAAGAAGGACCUGAUCAUCAAGCUUCCAAAGUACUCCCUGUUCGAGCUGGAGAAUGGGCGGAAGCGGAUGCUGGCC |
| | | UCCGCCGGUGAGCUGCAGAAGGGGAACGAGCUGGCACUUCCCUCAAAGUACGUGAACUUCCUGUACCUGGCCUCCCACUA |
| | | CGAGAAGCUGAAGGGGUCCCCAGAGGACAACGAGCAGAAGCAGCUGUUCGUGGAGCAGCACAAGCACUACCUGGACGAGA |
| | | UCAUCGAGCAGAUCUCCGAGUUCUCCAAGCGGGUGAUCCUGGCCGACGCCAAUCUCGACAAGGUGCUCAGCGCCUACAAC |
| | | AAGCACCGAGACAAGCCCAUCAGGGAGCAGGCCGAGAACAUCAUCCACCUGUUCACCCUGACCAAUCUCGGUGCCCCCGCU |
| | | GCCUUCAAGUACUUCGACACCACCAUCGACCGGAAGCGGUACACCUCGACUAAGGAAGUCCUGGACGCCACCCUGAUCCAC |
| | | CAGAGCAUCACCGGCCUGUACGAGACCCGGAUCGACCUCAGCCAGCUGGGUGGCGACGGUGGUGGCUCCCCCAAGAAGAA |
| | | GCGGAAGGUGUAGCUGGUACUGCAUGCACGCAAUGCUAGCUGCCCCUUUCCCGUCCUGGGUACCCCGAGUCUCCCCCGACC |
| | | UCGGGUCCCAGGUAUGCUCCCACCUCCACCUGCCCCACUCACCACCUCUGCUAGUUCCAGACACCUCCCAAGCACGCAGCA |
| | | AUGCAGCUCAAAACGCUUAGCCUAGCCACACCCCCACGGGAAACAGCAGUGAUUAACCUUUAGCAAUAAACGAAAGUUUA |
| | | ACUAAGCUAUACUAACCCCAGGGUUGGUCAAUUUCGUGCCAGCCACACCCUCGAGAAAAAAAAAAAAAAAAAAAAAAAAA |
| | | AAAAAAAAAAAAAAAAAAAAAAAAAAAAAAAAAAAAAAAAAAAAAAAAAAAAAAAAAAAAAAAAAAAAAAAAAAAUCU |
| | | AG |
| 201 | Cas9 mRNA | GGGAAGCUCAGAAUAAACGCUCAACUUUGGCCGGAUCUGCCACCAUGGACAAGAAGUACAGCAUCGGCCUGGACAUCGGC |
| | transcript | ACCAACUCCGUUGGCUGGGCUGUGAUCACCGACGAGUACAAGGUUCCCUCAAAGAAGUUCAAGGUGCUGGGCAACACCGA |
| | comprising SEQ | CCGGCACAGCAUCAAGAAGAAUCUCAUCGGUGCACUGCUGUUCGACUCCGGUGAGACCGCCGAAGCCACCCGGCUGAAGC |
| | ID No: 46 and | GGACCGCCCGCCGGCGGUACACCCGGCGGAAGAACCGGAUCUGCUACCUGCAGGAGAUCUUCUCCAACGAGAUGGCCAAG |
| | comprising SEQ | GUGGACGACUCCUUCUUCCACCGGCUGGAGGAGUCCUUCCUGGUGGAGGAGGACAAGAAGCACGAGCGGCACCCCAUCUU |
| | ID No: 203 | CGGCAACAUCGUGGACGAAGUCGCCUACCACGAGAAGUACCCCACCAUCUACCACCUGCGGAAGAAGCUGGUGGACUCGA |
| | | CUGACAAGGCCGACCUGCGGCUGAUCUACCUGGCACUGGCCCACAUGAUAAAGUUCCGGGGCCACUUCCUGAUCGAGGGC |
| | | GACCUGAACCCUGACAACUCCGACGUGGACAAGCUGUUCAUCCAGCUGGUGCAGACCUACAACCAGCUGUUCGAGGAGAA |
| | | CCCCAUCAACGCCUCCGGCGUGGACGCCAAGGCCAUCCUCAGCGCCCGCCUCAGCAAGUCCCGGCGGCUGGAGAAUCUCAU |
| | | CGCCCAGCUUCCAGGUGAGAAGAAGAAUGGGCUGUUCGGCAAUCUCAUCGCACUCAGCCUGGGCCUGACUCCCAACUUCA |
| | | AGUCCAACUUCGACCUGGCCGAGGACGCCAAGCUGCAGCUCAGCAAGGACACCUACGACGACGACCUGGACAAUCUCCUGG |
| | | CCCAGAUCGGCGACCAGUACGCCGACCUGUUCCUGGCUGCCAAGAAUCUCAGCGACGCCAUCCUGCUCAGCGACAUCCUGC |
| | | GGGUGAACACAGAGAUCACCAAGGCCCCCCUCAGCGCCUCCAUGAUAAAGCGGUACGACGAGCACCACCAGGACCUGACCC |
| | | UGCUGAAGGCACUGGUGCGGCAGCAGCUUCCAGAGAAGUACAAGGAGAUCUUCUUCGACCAGAGCAAGAAUGGGUACGCC |
| | | GGGUACAUCGACGGUGGUGCCUCCCAGGAGGAGUUCUACAAGUUCAUCAAGCCCAUCCUGGAGAAGAUGGACGGCACAGA |
| | | GGAGCUGCUGGUGAAGCUGAACAGGGAGGACCUGCUGCGGAAGCAGCGGACCUUCGACAAUGGGAGCAUCCCCCACCAGA |
| | | UCCACCUGGGUGAGCUGCACGCCAUCCUGCGGCGGCAGGAGGACUUCUACCCCUUCCUGAAGGACAACAGGGAGAAGAUC |
| | | GAGAAGAUCCUGACCUUCCGGAUCCCCUACUACGUUGGCCCCCUGGCCCGCGGCAACUCCCGGUUCGCCUGGAUGACCCGG |
| | | AAGUCCGAGGAGACCAUCACUCCCUGGAACUUCGAGGAAGUCGUGGACAAGGGUGCCUCCGCCCAGAGCUUCAUCGAGCG |
| | | GAUGACCAACUUCGACAAGAAUCUUCCAAACGAGAAGGUGCUUCCAAAGCACUCCCUGCUGUACGAGUACUUCACCGUGU |
| | | ACAACGAGCUGACCAAGGUGAAGUACGUGACAGAGGGCAUGCGGAAGCCCGCCUUCCUCAGCGGUGAGCAGAAGAAGGCC |
| | | AUCGUGGACCUGCUGUUCAAGACCAACCGGAAGGUGACCGUGAAGCAGCUGAAGGAGGACUACUUCAAGAAGAUCGAGUG |
| | | CUUCGACUCCGUGGAGAUCUCCGGCGUGGAGGACCGGUUCAACGCCUCCCUGGGCACCUACCACGACCUGCUGAAGAUCAU |
| | | CAAGGACAAGGACUUCCUGGACAACGAGGAGAACGAGGACAUCCUGGAGGACAUCGUGCUGACCCUGACCCUGUUCGAGG |
| | | ACAGGGAGAUGAUAGAGGAGCGGCUGAAGACCUACGCCCACCUGUUCGACGACAAGGUGAUGAAGCAGCUGAAGCGGCGG |
| | | CGGUACACCGGCUGGGGCCGGCUCAGCCGGAAGCUGAUCAAUGGGAUCCGAGACAAGCAGAGCGGCAAGACCAUCCUGGA |
| | | CUUCCUGAAGUCCGACGGCUUCGCCAACCGGAACUUCAUGCAGCUGAUCCACGACGACUCCCUGACCUUCAAGGAGGACAU |
| | | CCAGAAGGCCCAGGUCAGCGGCCAGGGCGACUCCCUGCACGAGCACAUCGCCAAUCUCGCCGGGUCCCCCGCCAUCAAGAA |
| | | GGGGAUCCUGCAGACCGUGAAGGUGGUGGACGAGCUGGUGAAGGUGAUGGGCCGGCACAAGCCAGAGAACAUCGUGAUCG |
| | | AGAUGGCCAGGGAGAACCAGACCACUCAAAAGGGGCAGAAGAACUCCAGGGAGCGGAUGAAGCGGAUCGAGGAGGGCAUC |
| | | AAGGAGCUGGGCUCCCAGAUCCUGAAGGAGCACCCCGUGGAGAACACUCAACUGCAGAACGAGAAGCUGUACCUGUACUA |
| | | CCUGCAGAAUGGGCGAGACAUGUACGUGGACCAGGAGCUGGACAUCAACCGGCUCAGCGACUACGACGUGGACCACAUCG |
| | | UUCCCCAGAGCUUCCUGAAGGACGACAGCAUCGACAACAAGGUGCUGACCCGGUCCGACAAGAACCGGGGCAAGUCCGAC |
| | | AACGUUCCCUCAGAGGAAGUCGUGAAGAAGAUGAAGAACUACUGGCGGCAGCUGCUGAACGCCAAGCUGAUCACUCAACG |
| | | GAAGUUCGACAAUCUCACCAAGGCCGAGCGGGGUGGCCUCAGCGAGCUGGACAAGGCCGGGUUCAUCAAGCGGCAGCUGG |
| | | UGGAGACCCGGCAGAUCACCAAGCACGUGGCCCAGAUCCUGGACUCCCGGAUGAACACCAAGUACGACGAGAACGACAAG |
| | | CUGAUCAGGGAAGUCAAGGUGAUCACCCUGAAGUCCAAGCUGGUCAGCGACUUCCGGAAGGACUUCCAGUUCUACAAGGU |
| | | GAGGGAGAUCAACAACUACCACCACGCCCACGACGCCUACCUGAACGCUGUGGUUGGCACCGCACUGAUCAAGAAGUACCC |
| | | CAAGCUGGAGUCCGAGUUCGUGUACGGCGACUACAAGGUGUACGACGUGCGGAAGAUGAUAGCCAAGUCCGAGCAGGAGA |
| | | UCGGCAAGGCCACCGCCAAGUACUUCUUCUACUCCAACAUCAUGAACUUCUUCAAGACAGAGAUCACCCUGGCCAAUGGU |
| | | GAGAUCCGGAAGCGGCCCCUGAUCGAGACCAAUGGUGAGACCGGUGAGAUCGUGUGGGACAAGGGGCGAGACUUCGCCAC |
| | | CGUGCGGAAGGUGCUCAGCAUGCCCCAGGUGAACAUCGUGAAGAAGACAGAAGUCCAGACCGGUGGCUUCUCCAAGGAGA |
| | | GCAUCCUUCCAAAGCGGAACUCCGACAAGCUGAUCGCCCGCAAGAAGGACUGGGACCCCAAGAAGUACGGUGGCUUCGAC |
| | | UCCCCCACCGUGGCCUACUCCGUGCUGGUGGUGGCCAAGGUGGAGAAGGGGAAGUCCAAGAAGCUGAAGUCCGUGAAGGA |
| | | GCUGCUGGGCAUCACCAUCAUGGAGCGGUCCUCCUUCGAGAAGAACCCCAUCGACUUCCUGGAAGCCAAGGGGUACAAGG |
| | | AAGUCAAGAAGGACCUGAUCAUCAAGCUUCCAAAGUACUCCCUGUUCGAGCUGGAGAAUGGGCGGAAGCGGAUGCUGGCC |
| | | UCCGCCGGUGAGCUGCAGAAGGGGAACGAGCUGGCACUUCCCUCAAAGUACGUGAACUUCCUGUACCUGGCCUCCCACUA |
| | | CGAGAAGCUGAAGGGGUCCCCAGAGGACAACGAGCAGAAGCAGCUGUUCGUGGAGCAGCACAAGCACUACCUGGACGAGA |
| | | UCAUCGAGCAGAUCUCCGAGUUCUCCAAGCGGGUGAUCCUGGCCGACGCCAAUCUCGACAAGGUGCUCAGCGCCUACAAC |
| | | AAGCACCGAGACAAGCCCAUCAGGGAGCAGGCCGAGAACAUCAUCCACCUGUUCACCCUGACCAAUCUCGGUGCCCCCGCU |
| | | GCCUUCAAGUACUUCGACACCACCAUCGACCGGAAGCGGUACACCUCGACUAAGGAAGUCCUGGACGCCACCCUGAUCCAC |
| | | CAGAGCAUCACCGGCCUGUACGAGACCCGGAUCGACCUCAGCCAGCUGGGUGGCGACGGUGGUGGCUCCCCCAAGAAGAA |
| | | GCGGAAGGUGUAGCAAGCACGCAGCAAUGCAGCUCAAAACGCUUAGCCUAGCCACACCCCCACGGGAAACAGCAGUGAUU |
| | | AACCUUUAGCAAUAAACGAAAGUUUAACUAAGCUAUACUAACCCCAGGGUUGGUCAAUUUCGUGCCAGCCACACCCUGGU |
| | | ACUGCAUGCACGCAAUGCUAGCUGCCCCUUUCCCGUCCUGGGUACCCCGAGUCUCCCCCGACCUCGGGUCCCAGGUAUGCU |
| | | CCCACCUCCACCUGCCCCACUCACCACCUCUGCUAGUUCCAGACACCUCCCUCGAGAAAAAAAAAAAAAAAAAAAAAAAAA |
| | | AAAAAAAAAAAAAAAAAAAAAAAAAAAAAAAAAAAAAAAAAAAAAAAAAAAAAAAAAAAAAAAAAAAAAAAAAAAUCU |
| | | AG |
| 202 | Exemplary 3′ | CTGGTACTGCATGCACGCAATGCTAGCTGCCCCTTTCCCGTCCTGGGTACCCCGAGTCTCCCCCGACCTCGGGTCCCAGGTATG |
| | UTR | CTCCCACCTCCACCTGCCCCACTCACCACCTCTGCTAGTTCCAGACACCTCCCAAGCACGCAGCAATGCAGCTCAAAACGCTTA |
| | | GCCTAGCCACACCCCCACGGGAAACAGCAGTGATTAACCTTTAGCAATAAACGAAAGTTTAACTAAGCTATACTAACCCCAGG |
| | | GTTGGTCAATTTCGTGCCAGCCACACC |
| 203 | Exemplary 3′ | CAAGCACGCAGCAATGCAGCTCAAAACGCTTAGCCTAGCCACACCCCCACGGGAAACAGCAGTGATTAACCTTTAGCAATAA |
| | UTR | ACGAAAGTTTAACTAAGCTATACTAACCCCAGGGTTGGTCAATTTCGTGCCAGCCACACCCTGGTACTGCATGCACGCAATGC |
| | | TAGCTGCCCCTTTCCCGTCCTGGGTACCCCGAGTCTCCCCCGACCTCGGGTCCCAGGTATGCTCCCACCTCCACCTGCCCCACT |
| | | CACCACCTCTGCTAGTTCCAGACACCTCC |
| 204 | Exemplary 3′ | ACCAGCCTCAAGAACACCCGAATGGAGTCTCTAAGCTACATAATACCAACTTACACTTTACAAAATGTTGTCCCCCAAAATGT |
| | UTR | AGCCATTCGTATCTGCTCCTAATAAAAAGAAAGTTTCTTCACATTCTACCAGCCTCAAGAACACCCGAATGGAGTCTCTAAGCT |
| | | ACATAATACCAACTTACACTTTACAAAATGTTGTCCCCCAAAATGTAGCCATTCGTATCTGCTCCTAATAAAAAGAAAGTTTCT |
| | | TCACATTCT |

We claim:

1. A polynucleotide comprising (i) an open reading frame (ORF) encoding a polypeptide, wherein at least 1.03% of the codon pairs in the ORF are codon pairs shown in Table 1; or (ii) an open reading frame (ORF) encoding a polypeptide, wherein at least 1% of the codon pairs in the ORF are codon pairs shown in Table 1 and the ORF does not encode an RNA-guided DNA binding agent.

2. A polynucleotide comprising an open reading frame (ORF) encoding a polypeptide, wherein the ORF comprises a sequence with at least 95% identity to any one of SEQ ID NOs: 6-10, 29, 46, 69-73, 90-93, 96-99, 102-105, 108-111, 114-117, 120-123, 126-129, or 132-143, optionally wherein identity is determined without regard to the start and stop codons of the ORF.

3. A polynucleotide comprising an open reading frame (ORF) encoding a polypeptide, wherein at least 75%, 80%, 85%, 90%, 95%, 98%, 99%, or 100% of the codons in the ORF are (i) codons listed in Table 5, or (ii) codons listed in Table 6, and wherein the polypeptide is not an RNA-guided DNA binding agent.

4. The polynucleotide of any one of claims 1-3, wherein the repeat content of the ORF is less than or equal to 23.3%.

5. The polynucleotide of any one of claims 1-4, wherein the GC content of the ORF is greater than or equal to 55%.

6. A polynucleotide comprising an open reading frame (ORF) encoding a polypeptide, wherein the repeat content of the ORF is less than or equal to 23.3% and the GC content of the ORF is greater than or equal to 55%.

7. The polynucleotide of any one of claims 2-6, wherein at least 1.03% of the codon pairs in the ORF are codon pairs shown in Table 1.

8. The polynucleotide of any one of claims 1-7, wherein less than or equal to 0.9% of the codon pairs in the ORF are codon pairs shown in Table 2.

9. The polynucleotide of any one of claims 1-8, wherein at least 60%, 65%, 70%, or 75% of the codon in the ORF are codon shown in Table 3.

10. The polynucleotide of any one of claims 1-9, wherein less than or equal to 20% of the codons in the ORF are codons shown in Table 4.

11. The polynucleotide of any one of claims 1-10, wherein at least 1.05% of the codon pairs in the ORF are codon pairs shown in Table 1.

12. The polynucleotide of any one of claims 1-11, wherein less than or equal to 10% of the codon pairs in the ORF are codon pairs shown in Table 1.

13. The polynucleotide of any one of claims 1-12, wherein less than or equal to 0.9% of the codon pairs in the ORF are codon pairs shown in Table 2.

14. The polynucleotide of any one of claims 1-13, wherein the GC content of the ORF is greater than or equal to 56%.

15. The polynucleotide of any one of claims 1-14, wherein the GC content of the ORF is less than or equal to 63%.

16. The polynucleotide of any one of claims 1-15, wherein the repeat content of the ORF is less than or equal to 23.2%.

17. The polynucleotide of any one of claims 1-16, wherein the repeat content of the ORF is greater than or equal to 20%.

18. The polynucleotide of any one of claims 1-17, wherein less than or equal to 15% of the codons in the ORF are codons shown in Table 4.

19. The polynucleotide of any one of claims 1-18, wherein at least 76% of the codons in the ORF are codons shown in Table 3.

20. The polynucleotide of any one of claims 1-19, wherein less than or equal to 87% of the codons in the ORF are codons shown in Table 3.

21. The polynucleotide of any one of claims 1-20, wherein the ORF has a uridine content ranging from its minimum uridine content to 101%, 102%, 103%, 105%, 110%, 115%, 120%, 125%, 130%, 135%, 140%, 145%, or 150% of the minimum uridine content.

22. The polynucleotide of any one of claims 1-21, wherein the ORF has an A+U content ranging from its minimum A+U content to 101%, 102%, 103%, 105%, 110%, 115%, 120%, 125%, 130%, 135%, 140%, 145%, or 150% of the minimum A+U content.

23. The polynucleotide of any one of claims 1-22, wherein the ORF has a GC content in the range of 55%-65%, such as 55%-57%, 57%-59%, 59-61%, 61-63%, or 63-65%.

24. The polynucleotide of any one of claims 1-23, wherein the ORF has a repeat content ranging from its minimum repeat content to 101%, 102%, 103%, 105%, 110%, 115%, 120%, 125%, 130%, 135%, 140%, 145%, or 150% of the minimum repeat content.

25. The polynucleotide of any one of claims 1-24, wherein the ORF has a repeat content of 22%-27%, such as 22%-23%, 22.3%-23%, 23%-24%, 24%-25%, 25%-26%, or 26%-27%.

26. The polynucleotide of any one of claims 1-25, wherein the polypeptide has a length of 30 amino acids, optionally wherein the polypeptide has a length of at least 50 amino acids.

27. The polynucleotide of any one of claims 1-26, wherein the polypeptide has a length of at least 100 amino acids.

28. The polynucleotide of any one of claims 1-27, wherein the length of the polypeptide is less than or equal to 5000 amino acids.

29. The polynucleotide of any one of claims 1-28, wherein the polypeptide comprises a sequence with at least 90%, 95%, 96%, 97%, 98%, 99%, 99.5%, or 100% identity to any one of SEQ ID NOs: 6-10, 29, 46, 69-73, 90-93, 96-99, 102-105, 108-111, 114-117, 120-123, 126-129, or 134-143.

30. The polynucleotide of any one of claims 1-29, wherein the polynucleotide comprises a sequence with at least 90%, 95%, 96%, 97%, 98%, 99%, 99.5%, or 100% identity to any one of SEQ ID NOs: 16-20, 78-80, 194-197, or 200-201.

31. The polynucleotide of any one of claims 1-30, wherein the ORF encodes an RNA-guided DNA binding agent.

32. The polynucleotide of claim 31, wherein the RNA-guided DNA-binding agent has double-stranded endonuclease activity.

33. The polynucleotide of claim 31, wherein the RNA-guided DNA-binding agent has nickase activity.

34. The polynucleotide of claim 31, wherein the RNA-guided DNA-binding agent comprises a dCas DNA binding domain.

35. The polynucleotide of any one of claims 1-34, wherein the ORF encodes an S. pyogenes Cas9.

36. The polynucleotide of any one of claims 1-35, wherein the ORF encodes an endonuclease.

37. The polynucleotide of any one of claims 1-36, wherein the ORF encodes a serine protease inhibitor or Serpin family member, optionally wherein the ORF encodes a Serpin Family A Member 1.

38. The polynucleotide of any one of claims 1-37, wherein the ORF encodes a hydroxylase; carbamoyltransferase; glucosylceramidase; galactosidase; dehydrogenase; receptor; or neurotransmitter receptor.

39. The polynucleotide of any one of claims 1-38, wherein the ORF encodes a phenylalanine hydroxylase; an ornithine carbamoyltransferase; a fumarylacetoacetate hydrolase; a glucosylceramidase beta; an alpha galactosidase; a transthyretin; a glyceraldehyde-3-phosphate dehydrogenase; a gamma-aminobutyric acid (GABA) receptor subunit (such as a GABA Type A Receptor Delta Subunit).

40. The polynucleotide of any one of claims 1-39, wherein the polynucleotide further comprises a 5′ UTR with at least 90% identity to any one of SEQ ID NOs: 177-181 or 190-192; and/or a 3′ UTR with at least 90% identity to any one of SEQ ID NOs: 182-186 or 202-204.

41. The polynucleotide of any one of claims 1-40, wherein the polynucleotide further comprises a 5′ cap selected from Cap0, Cap1, and Cap2.

42. The polynucleotide of any one of claims 1-41, wherein the open reading frame has codons that increase translation of the polynucleotide in a mammal.

43. The polynucleotide of any one of claims 1-42, wherein the encoded polypeptide comprises a nuclear localization signal (NLS).

44. The polynucleotide of claim 43, wherein the NLS comprises a sequence having at least 80%, 85%, 90%, or 95% identity to any one of SEQ ID NOs: 163-176.

45. The polynucleotide of any one of claims 1-44, wherein the polypeptide encodes an RNA-guided DNA-binding agent and the RNA-guided DNA-binding agent further comprises a heterologous functional domain.

46. The polynucleotide of any of claims 1-45, wherein at least 10%, at least 20%, at least 30%, at least 40%, at least 50%, at least 60%, at least 70%, at least 80%, at least 90%, at least 95%, at least 98%, at least 99%, or 100% of the uridine is substituted with a modified uridine, optionally, wherein the modified uridine is one or more of N1-methyl-pseudouridine, pseudouridine, 5-methoxyuridine, or 5-iodouridine.

47. The polynucleotide of claim 46, wherein 15% to 45%, 45% to 55%, 55% to 65%, 65% to 75%, 75% to 85%, 85% to 95%, or 90% to 100% of the uridine is substituted with the modified uridine, optionally wherein the modified uridine is N1-methyl-pseudouridine.

48. The polynucleotide of any one of claims 1-47, wherein the polynucleotide is an mRNA.

49. The polynucleotide of any one of claims 1-48, wherein the polynucleotide is an expression construct comprising a promoter operably linked to the ORF.

50. A plasmid comprising the expression construct of claim 49.

51. A host cell comprising the expression construct of claim 49 or the plasmid of claim 50.

52. A method of preparing an mRNA comprising contacting the expression construct of claim 49 or the plasmid of claim 50 with an RNA polymerase under conditions permissive for transcription of the mRNA, optionally wherein the contacting step is performed in vitro.

53. A method of expressing a polypeptide, comprising contacting a cell with the polynucleotide of any one of claims 1-49.

54. The method of claim 53, wherein the cell is in a mammalian subject, optionally wherein the subject is human.

55. The method of claim 53, wherein the cell is a cultured cell and/or the contacting is performed in vitro.

56. The method of any one of claims 53-55, wherein the cell is a human cell.

57. A composition comprising a polynucleotide according to any one of claims 1-49 and at least one guide RNA, wherein the polynucleotide encodes an RNA-guided DNA binding agent.

58. A lipid nanoparticle comprising a polynucleotide according to any one of claims 1-49.

59. A pharmaceutical composition comprising a polynucleotide according to any one of claims 1-49 and a pharmaceutically acceptable carrier.

60. The lipid nanoparticle of claim 58 or the pharmaceutical composition of claim 59, wherein the polynucleotide encodes an RNA-guided DNA binding agent and the lipid nanoparticle or pharmaceutical composition further comprises at least one guide RNA.

61. A method of genome editing or modifying a target gene comprising contacting a cell with the polynucleotide, expression construct, composition, or lipid nanoparticle according to any one of claim 1-49 or 57-60, wherein the polynucleotide encodes an RNA-guided DNA binding agent.

62. Use of the polynucleotide, expression construct, composition, or lipid nanoparticle according to any one of claim 1-49 or 57-60 for genome editing or modifying a target gene, wherein the polynucleotide encodes an RNA-guided DNA binding agent.

63. Use of the polynucleotide, expression construct, composition, or lipid nanoparticle according to any one of claim 1-49 or 57-60 for the manufacture of a medicament for genome editing or modifying a target gene, wherein the polynucleotide encodes an RNA-guided DNA binding agent.

64. The method or use of any one of claims 61-63, wherein the genome editing or modification of the target gene occurs in a liver cell, optionally wherein the liver cell is a hepatocyte.

65. A method of generating an open reading frame (ORF) sequence encoding a polypeptide, the method comprising:
a) providing a polypeptide sequence of interest;
b) assigning a codon for each amino acid position of the polypeptide sequence, wherein if the amino acid position is a member of a dipeptide shown in Table 1, then the codon pair for that dipeptide is used, but if the amino acid position is a member of more than one dipeptide shown in Table 1 and the codon pairs for those dipeptides provide different codons for the position or the amino acid position is not a member of a dipeptide shown in Table 1, then one or more of the following is performed:
i. selecting a codon from a wild-type sequence encoding the polypeptide if a naturally occurring polypeptide is encoded;
ii. if the amino acid is a member of more than one dipeptide shown in Table 1 and the codon pairs for those dipeptides provide different codons for the position, eliminating codons that appear in Table 4 and/or that would result in the presence of a codon pair shown in Table 2, and/or selecting a codon that appears in Table 3;
iii. using a codon set of Table 5, 6, or 7 to supply the codon for the amino acid position, optionally wherein if steps (i) and/or (ii) are performed then step (iii) is performed if a unique codon for the amino acid position has not been provided; and/or
iv. selecting a codon that (1) minimizes uridine content, (2) minimizes repeat content, and/or (3) maximizes GC content.

66. The method of claim 65, wherein for at least one amino acid, Table 1 does not provide a unique codon at a given amino acid position, optionally wherein there are (1) conflicting codons in overlapping dipeptides; (2) multiple possible codons that corresponds to a given dipeptide; or (3) no codon that corresponds to a given dipeptide.

67. The method of claim 65 or 66, wherein step (b)(ii) comprises performing one or more of the following:
a. selecting a codon that appears in Table 3; and/or
b. eliminating codon(s) that would result in the presence of a codon pair in Table 2 and/or codon(s) that appear in Table 4,
wherein one or more of the above steps are performed in any order and the steps are terminated when a single codon for the amino acid is provided.

68. The method of any one of claims 65-67, wherein step (b)(ii) comprises selecting a codon that appears in Table 3, optionally wherein if one or more steps of claim 234 are performed, then the one or more steps of claim 234 are performed in any order relative to selecting a codon that appears in Table 3.

69. The method of any one of claims 65-68, wherein step (b)(ii) further comprises:
a. eliminating codons that would result in the presence of a codon pair in Table 2; and
b. if more than one possible codon remains after step (a), eliminating codons that do not appear in Table 3 and/or eliminating codons that appear in Table 4.

70. The method of any one of claims 65-69, wherein step (b)(ii) further comprises:
a. eliminating codons that do not appear in Table 3 and/or eliminating codons that appear in Table 4; and
b. if more than one possible codon remains after step (a), eliminating codons that would result in the presence of a codon pair in Table 2.

71. The method of any one of claims 65-70, wherein step (b) comprises performing one or more of the following:
a. selecting the codon that minimizes uridine content;
b. selecting the codon that minimizes repeat content;
c. selecting the codon that maximizes GC content,
wherein one or more of the above steps are performed in any order, optionally wherein the steps are terminated when a single codon for the amino acid is provided.

72. The method of claim 71, wherein step (b) comprises performing at least one of the following and continuing to perform the following steps, optionally wherein each of the following steps (i)-(iii) is performed:
i. selecting the codon that minimizes uridine content;
ii. if more than one possible codon remains after step (a), selecting the codon that minimizes repeat content;
iii. if more than one possible codon remains after step (b), Selecting the codon that maximizes GC content.

73. The method of any one of claims 65-72, wherein no codons remain after performing step (b)(ii) for at least one position that can be encoded by more than one codon, and the following steps are performed on a plurality of codons that encode the amino acid at the position:
i. selecting the codon that minimizes uridine content;
ii. if more than one possible codon remains after step (i), selecting the codon that minimizes repeat content;
iii. if more than one possible codon remains after step (ii), selecting the codon that maximizes GC content.

74. The method of any one of claims 65-73, wherein a plurality of codons remain after performing step (b)(ii) for at least one position that can be encoded by more than one codon, and the following steps are performed on the plurality of codons:
i. selecting the codon that minimizes uridine content;
ii. if more than one possible codon remains after step (i), selecting the codon that minimizes repeat content;
iii. if more than one possible codon remains after step (ii), selecting the codon that maximizes GC content.

75. The method of claim 73 or 74, wherein the method comprises selecting the codon that maximizes GC content in at least one position.

76. The method of any one of claims 65-75, further comprising selecting a one-to-one codon set shown in Table 5, 6, or 7, and assigning a codon for at least one position from the set.

77. The method of any one of claims 65-76, further comprising:
a. generating a set of all available codons for the amino acid to be encoded by at least one position;
b. applying one or more of the steps recited in claims 233-243.

78. The method of any one of claims 65-77, wherein at least step (b) of the method is computer-implemented.

79. The method of any one of claims 65-78, further comprising synthesizing a polynucleotide comprising the ORF, optionally wherein the polynucleotide is an mRNA.

80. The method of any one of claims 65-79, wherein the RNA-guided DNA-binding agent has double-stranded endonuclease activity.

81. The method of any one of claims 65-80, wherein the ORF encodes a polypeptide having at least 90% identity the amino acid sequence of any one of SEQ ID NOs: 1, 74, 88, 94, 100, 106, 112, 118, 124, 130, 161, or 162.


SEQUENCE LISTING (204 sequences)

SEQ ID NO: 1 - protein, 1379 aa, Artificial Sequence
  Synthetic: Cas9 amino acid sequence
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKV

SEQ ID NO: 2 - RNA, 4491 nt, Artificial Sequence
  Synthetic: Cas9 transcript with ORF having low U content
gggucccgcagucggcguccagcggcucugcuuguucgugugugugucguugcaggccuuauucggauccgccaccauggacaagaaguacagcaucggacuggacaucggaacaaacagcgucggaugggcagucaucacagacgaauacaaggucccgagcaagaaguucaagguccugggaaacacagacagacacagcaucaagaagaaccugaucggagcacugcuguucgacagcggagaaacagcagaagcaacaagacugaagagaacagcaagaagaagauacacaagaagaaagaacagaaucugcuaccugcaggaaaucuucagcaacgaaauggcaaaggucgacgacagcuucuuccacagacuggaagaaagcuuccuggucgaagaagacaagaagcacgaaagacacccgaucuucggaaacaucgucgacgaagucgcauaccacgaaaaguacccgacaaucuaccaccugagaaagaagcuggucgacagcacagacaaggcagaccugagacugaucuaccuggcacuggcacacaugaucaaguucagaggacacuuccugaucgaaggagaccugaacccggacaacagcgacgucgacaagcuguucauccagcugguccagacauacaaccagcuguucgaagaaaacccgaucaacgcaagcggagucgacgcaaaggcaauccugagcgcaagacugagcaagagcagaagacuggaaaaccugaucgcacagcugccgggagaaaagaagaacggacuguucggaaaccugaucgcacugagccugggacugacaccgaacuucaagagcaacuucgaccuggcagaagacgcaaagcugcagcugagcaaggacacauacgacgacgaccuggacaaccugcuggcacagaucggagaccaguacgcagaccuguuccuggcagcaaagaaccugagcgacgcaauccugcugagcgacauccugagagucaacacagaaaucacaaaggcaccgcugagcgcaagcaugaucaagagauacgacgaacaccaccaggaccugacacugcugaaggcacuggucagacagcagcugccggaaaaguacaaggaaaucuucuucgaccagagcaagaacggauacgcaggauacaucgacggaggagcaagccaggaagaauucuacaaguucaucaagccgauccuggaaaagauggacggaacagaagaacugcuggucaagcugaacagagaagaccugcugagaaagcagagaacauucgacaacggaagcaucccgcaccagauccaccugggagaacugcacgcaauccugagaagacaggaagacuucuacccguuccugaaggacaacagagaaaagaucgaaaagauccugacauucagaaucccguacuacgucggaccgcuggcaagaggaaacagcagauucgcauggaugacaagaaagagcgaagaaacaaucacaccguggaacuucgaagaagucgucgacaagggagcaagcgcacagagcuucaucgaaagaaugacaaacuucgacaagaaccugccgaacgaaaagguccugccgaagcacagccugcuguacgaauacuucacagucuacaacgaacugacaaaggucaaguacgucacagaaggaaugagaaagccggcauuccugagcggagaacagaagaaggcaaucgucgaccugcuguucaagacaaacagaaaggucacagucaagcagcugaaggaagacuacuucaagaagaucgaaugcuucgacagcgucgaaaucagcggagucgaagacagauucaacgcaagccugggaacauaccacgaccugcugaagaucaucaaggacaaggacuuccuggacaacgaagaaaacgaagacauccuggaagacaucguccugacacugacacuguucgaagacagagaaaugaucgaagaaagacugaagacauacgcacaccuguucgacgacaaggucaugaagcagcugaagagaagaagauacacaggauggggaagacugagcagaaagcugaucaacggaaucagagacaagcagagcggaaagacaauccuggacuuccugaagagcgacggauucgcaaacagaaacuucaugcagcugauccacgacgacagccugacauucaaggaagacauccagaaggcacaggucagcggacagggagacagccugcacgaacacaucgcaaaccuggcaggaagcccggcaaucaagaagggaauccugcagacagucaaggucgucgacgaacuggucaaggucaugggaagacacaagccggaaaacaucgucaucgaaauggcaagagaaaaccagacaacacagaagggacagaagaacagcagagaaagaaugaagagaaucgaagaaggaaucaaggaacugggaagccagauccugaaggaacacccggucgaaaacacacagcugcagaacgaaaagcuguaccuguacuaccugcagaacggaagagacauguacgucgaccaggaacuggacaucaacagacugagcgacuacgacgucgaccacaucgucccgcagagcuuccugaaggacgacagcaucgacaacaagguccugacaagaagcgacaagaacagaggaaagagcgacaacgucccgagcgaagaagucgucaagaagaugaagaacuacuggagacagcugcugaacgcaaagcugaucacacagagaaaguucgacaaccugacaaaggcagagagaggaggacugagcgaacuggacaaggcaggauucaucaagagacagcuggucgaaacaagacagaucacaaagcacgucgcacagauccuggacagcagaaugaacacaaaguacgacgaaaacgacaagcugaucagagaagucaaggucaucacacugaagagcaagcuggucagcgacuucagaaaggacuuccaguucuacaaggucagagaaaucaacaacuaccaccacgcacacgacgcauaccugaacgcagucgucggaacagcacugaucaagaaguacccgaagcuggaaagcgaauucgucuacggagacuacaaggucuacgacgucagaaagaugaucgcaaagagcgaacaggaaaucggaaaggcaacagcaaaguacuucuucuacagcaacaucaugaacuucuucaagacagaaaucacacuggcaaacggagaaaucagaaagagaccgcugaucgaaacaaacggagaaacaggagaaaucgucugggacaagggaagagacuucgcaacagucagaaagguccugagcaugccgcaggucaacaucgucaagaagacagaaguccagacaggaggauucagcaaggaaagcauccugccgaagagaaacagcgacaagcugaucgcaagaaagaaggacugggacccgaagaaguacggaggauucgacagcccgacagucgcauacagcguccuggucgucgcaaaggucgaaaagggaaagagcaagaagcugaagagcgucaaggaacugcugggaaucacaaucauggaaagaagcagcuucgaaaagaacccgaucgacuuccuggaagcaaagggauacaaggaagucaagaaggaccugaucaucaagcugccgaaguacagccuguucgaacuggaaaacggaagaaagagaaugcuggcaagcgcaggagaacugcagaagggaaacgaacuggcacugccgagcaaguacgucaacuuccuguaccuggcaagccacuacgaaaagcugaagggaagcccggaagacaacgaacagaagcagcuguucgucgaacagcacaagcacuaccuggacgaaaucaucgaacagaucagcgaauucagcaagagagucauccuggcagacgcaaaccuggacaagguccugagcgcauacaacaagcacagagacaagccgaucagagaacaggcagaaaacaucauccaccuguucacacugacaaaccugggagcaccggcagcauucaaguacuucgacacaacaaucgacagaaagagauacacaagcacaaaggaaguccuggacgcaacacugauccaccagagcaucacaggacuguacgaaacaagaaucgaccugagccagcugggaggagacggaggaggaagcccgaagaagaagagaaaggucuagcuagccaucacauuuaaaagcaucucagccuaccaugagaauaagagaaagaaaaugaagaucaauagcuuauucaucucuuuuucuuuuucguugguguaaagccaacacccugucuaaaaaacauaaauuucuuuaaucauuuugccucuuuucucugugcuucaauuaauaaaaaauggaaagaaccucgagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 3 - RNA, 4405 nt, Artificial Sequence
  Synthetic: Cas9 transcript with ORF having low A content
gggaagcucagaauaaacgcucaacuuuggccggaucugccaccauggacaagaaguacuccaucggccuggacaucggcaccaacuccgugggcugggccgugaucaccgacgaguacaaggugcccuccaagaaguucaaggugcugggcaacaccgaccggcacuccaucaagaagaaccugaucggcgcccugcuguucgacuccggcgagaccgccgaggccacccggcugaagcggaccgcccggcggcgguacacccggcggaagaaccggaucugcuaccugcaggagaucuucuccaacgagauggccaagguggacgacuccuucuuccaccggcuggaggaguccuuccugguggaggaggacaagaagcacgagcggcaccccaucuucggcaacaucguggacgagguggccuaccacgagaaguaccccaccaucuaccaccugcggaagaagcugguggacuccaccgacaaggccgaccugcggcugaucuaccuggcccuggcccacaugaucaaguuccggggccacuuccugaucgagggcgaccugaaccccgacaacuccgacguggacaagcuguucauccagcuggugcagaccuacaaccagcuguucgaggagaaccccaucaacgccuccggcguggacgccaaggccauccuguccgcccggcuguccaagucccggcggcuggagaaccugaucgcccagcugcccggcgagaagaagaacggccuguucggcaaccugaucgcccugucccugggccugacccccaacuucaaguccaacuucgaccuggccgaggacgccaagcugcagcuguccaaggacaccuacgacgacgaccuggacaaccugcuggcccagaucggcgaccaguacgccgaccuguuccuggccgccaagaaccuguccgacgccauccugcuguccgacauccugcgggugaacaccgagaucaccaaggccccccuguccgccuccaugaucaagcgguacgacgagcaccaccaggaccugacccugcugaaggcccuggugcggcagcagcugcccgagaaguacaaggagaucuucuucgaccaguccaagaacggcuacgccggcuacaucgacggcggcgccucccaggaggaguucuacaaguucaucaagcccauccuggagaagauggacggcaccgaggagcugcuggugaagcugaaccgggaggaccugcugcggaagcagcggaccuucgacaacggcuccaucccccaccagauccaccugggcgagcugcacgccauccugcggcggcaggaggacuucuaccccuuccugaaggacaaccgggagaagaucgagaagauccugaccuuccggauccccuacuacgugggcccccuggcccggggcaacucccgguucgccuggaugacccggaaguccgaggagaccaucacccccuggaacuucgaggaggugguggacaagggcgccuccgcccaguccuucaucgagcggaugaccaacuucgacaagaaccugcccaacgagaaggugcugcccaagcacucccugcuguacgaguacuucaccguguacaacgagcugaccaaggugaaguacgugaccgagggcaugcggaagcccgccuuccuguccggcgagcagaagaaggccaucguggaccugcuguucaagaccaaccggaaggugaccgugaagcagcugaaggaggacuacuucaagaagaucgagugcuucgacuccguggagaucuccggcguggaggaccgguucaacgccucccugggcaccuaccacgaccugcugaagaucaucaaggacaaggacuuccuggacaacgaggagaacgaggacauccuggaggacaucgugcugacccugacccuguucgaggaccgggagaugaucgaggagcggcugaagaccuacgcccaccuguucgacgacaaggugaugaagcagcugaagcggcggcgguacaccggcuggggccggcugucccggaagcugaucaacggcauccgggacaagcaguccggcaagaccauccuggacuuccugaaguccgacggcuucgccaaccggaacuucaugcagcugauccacgacgacucccugaccuucaaggaggacauccagaaggcccagguguccggccagggcgacucccugcacgagcacaucgccaaccuggccggcucccccgccaucaagaagggcauccugcagaccgugaaggugguggacgagcuggugaaggugaugggccggcacaagcccgagaacaucgugaucgagauggcccgggagaaccagaccacccagaagggccagaagaacucccgggagcggaugaagcggaucgaggagggcaucaaggagcugggcucccagauccugaaggagcaccccguggagaacacccagcugcagaacgagaagcuguaccuguacuaccugcagaacggccgggacauguacguggaccaggagcuggacaucaaccggcuguccgacuacgacguggaccacaucgugccccaguccuuccugaaggacgacuccaucgacaacaaggugcugacccgguccgacaagaaccggggcaaguccgacaacgugcccuccgaggagguggugaagaagaugaagaacuacuggcggcagcugcugaacgccaagcugaucacccagcggaaguucgacaaccugaccaaggccgagcggggcggccuguccgagcuggacaaggccggcuucaucaagcggcagcugguggagacccggcagaucaccaagcacguggcccagauccuggacucccggaugaacaccaaguacgacgagaacgacaagcugauccgggaggugaaggugaucacccugaaguccaagcugguguccgacuuccggaaggacuuccaguucuacaaggugcgggagaucaacaacuaccaccacgcccacgacgccuaccugaacgccguggugggcaccgcccugaucaagaaguaccccaagcuggaguccgaguucguguacggcgacuacaagguguacgacgugcggaagaugaucgccaaguccgagcaggagaucggcaaggccaccgccaaguacuucuucuacuccaacaucaugaacuucuucaagaccgagaucacccuggccaacggcgagauccggaagcggccccugaucgagaccaacggcgagaccggcgagaucgugugggacaagggccgggacuucgccaccgugcggaaggugcuguccaugccccaggugaacaucgugaagaagaccgaggugcagaccggcggcuucuccaaggaguccauccugcccaagcggaacuccgacaagcugaucgcccggaagaaggacugggaccccaagaaguacggcggcuucgacucccccaccguggccuacuccgugcuggugguggccaagguggagaagggcaaguccaagaagcugaaguccgugaaggagcugcugggcaucaccaucauggagcgguccuccuucgagaagaaccccaucgacuuccuggaggccaagggcuacaaggaggugaagaaggaccugaucaucaagcugcccaaguacucccuguucgagcuggagaacggccggaagcggaugcuggccuccgccggcgagcugcagaagggcaacgagcuggcccugcccuccaaguacgugaacuuccuguaccuggccucccacuacgagaagcugaagggcucccccgaggacaacgagcagaagcagcuguucguggagcagcacaagcacuaccuggacgagaucaucgagcagaucuccgaguucuccaagcgggugauccuggccgacgccaaccuggacaaggugcuguccgccuacaacaagcaccgggacaagcccauccgggagcaggccgagaacaucauccaccuguucacccugaccaaccugggcgcccccgccgccuucaaguacuucgacaccaccaucgaccggaagcgguacaccuccaccaaggaggugcuggacgccacccugauccaccaguccaucaccggccuguacgagacccggaucgaccugucccagcugggcggcgacggcggcggcucccccaagaagaagcggaaggugugacuagcaccagccucaagaacacccgaauggagucucuaagcuacauaauaccaacuuacacuuuacaaaauguugucccccaaaauguagccauucguaucugcuccuaauaaaaagaaaguuucuucacauucucucgagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 4 - RNA, 100 nt, Artificial Sequence
  Synthetic: Guide RNA G000502
  modified_base (1)..(3): 2′-O-Me nucleotide
  modified_base (1)..(4): PS linkage
  modified_base (29)..(40): 2′-O-Me nucleotide
  modified_base (69)..(100): 2′-O-Me nucleotide
  modified_base (97)..(100): PS linkage
acacaaauaccaguccagcgguuuuagagcuagaaauagcaaguuaaaauaaggcuaguccguuaucaacuugaaaaaguggcaccgagucggugcuuuu

SEQ ID NO: 5 - RNA, 4140 nt, Artificial Sequence
  Synthetic: E-single enriched Cas9 ORF
auggacaagaaguacagcaucggccuggacaucggcacgaacagcgugggcugggccgugaucacggacgaguacaaggugcccagcaagaaguucaaggugcugggcaacacggaccggcacagcaucaagaagaaccugaucggcgcccugcuguucgacagcggcgagacggccgaggccacgcggcugaagcggacggcccggcggcgguacacgcggcggaagaaccggaucugcuaccugcaggagaucuucagcaacgagauggccaagguggacgacagcuucuuccaccggcuggaggagagcuuccugguggaggaggacaagaagcacgagcggcaccccaucuucggcaacaucguggacgagguggccuaccacgagaaguaccccacgaucuaccaccugcggaagaagcugguggacagcacggacaaggccgaccugcggcugaucuaccuggcccuggcccacaugaucaaguuccggggccacuuccugaucgagggcgaccugaaccccgacaacagcgacguggacaagcuguucauccagcuggugcagacguacaaccagcuguucgaggagaaccccaucaacgccagcggcguggacgccaaggccauccugagcgcccggcugagcaagagccggcggcuggagaaccugaucgcccagcugcccggcgagaagaagaacggccuguucggcaaccugaucgcccugagccugggccugacgcccaacuucaagagcaacuucgaccuggccgaggacgccaagcugcagcugagcaaggacacguacgacgacgaccuggacaaccugcuggcccagaucggcgaccaguacgccgaccuguuccuggccgccaagaaccugagcgacgccauccugcugagcgacauccugcgggugaacacggagaucacgaaggccccccugagcgccagcaugaucaagcgguacgacgagcaccaccaggaccugacgcugcugaaggcccuggugcggcagcagcugcccgagaaguacaaggagaucuucuucgaccagagcaagaacggcuacgccggcuacaucgacggcggcgccagccaggaggaguucuacaaguucaucaagcccauccuggagaagauggacggcacggaggagcugcuggugaagcugaaccgggaggaccugcugcggaagcagcggacguucgacaacggcagcaucccccaccagauccaccugggcgagcugcacgccauccugcggcggcaggaggacuucuaccccuuccugaaggacaaccgggagaagaucgagaagauccugacguuccggauccccuacuacgugggcccccuggcccggggcaacagccgguucgccuggaugacgcggaagagcgaggagacgaucacgcccuggaacuucgaggaggugguggacaagggcgccagcgcccagagcuucaucgagcggaugacgaacuucgacaagaaccugcccaacgagaaggugcugcccaagcacagccugcuguacgaguacuucacgguguacaacgagcugacgaaggugaaguacgugacggagggcaugcggaagcccgccuuccugagcggcgagcagaagaaggccaucguggaccugcuguucaagacgaaccggaaggugacggugaagcagcugaaggaggacuacuucaagaagaucgagugcuucgacagcguggagaucagcggcguggaggaccgguucaacgccagccugggcacguaccacgaccugcugaagaucaucaaggacaaggacuuccuggacaacgaggagaacgaggacauccuggaggacaucgugcugacgcugacgcuguucgaggaccgggagaugaucgaggagcggcugaagacguacgcccaccuguucgacgacaaggugaugaagcagcugaagcggcggcgguacacgggcuggggccggcugagccggaagcugaucaacggcauccgggacaagcagagcggcaagacgauccuggacuuccugaagagcgacggcuucgccaaccggaacuucaugcagcugauccacgacgacagccugacguucaaggaggacauccagaaggcccaggugagcggccagggcgacagccugcacgagcacaucgccaaccuggccggcagccccgccaucaagaagggcauccugcagacggugaaggugguggacgagcuggugaaggugaugggccggcacaagcccgagaacaucgugaucgagauggcccgggagaaccagacgacgcagaagggccagaagaacagccgggagcggaugaagcggaucgaggagggcaucaaggagcugggcagccagauccugaaggagcaccccguggagaacacgcagcugcagaacgagaagcuguaccuguacuaccugcagaacggccgggacauguacguggaccaggagcuggacaucaaccggcugagcgacuacgacguggaccacaucgugccccagagcuuccugaaggacgacagcaucgacaacaaggugcugacgcggagcgacaagaaccggggcaagagcgacaacgugcccagcgaggagguggugaagaagaugaagaacuacuggcggcagcugcugaacgccaagcugaucacgcagcggaaguucgacaaccugacgaaggccgagcggggcggccugagcgagcuggacaaggccggcuucaucaagcggcagcugguggagacgcggcagaucacgaagcacguggcccagauccuggacagccggaugaacacgaaguacgacgagaacgacaagcugauccgggaggugaaggugaucacgcugaagagcaagcuggugagcgacuuccggaaggacuuccaguucuacaaggugcgggagaucaacaacuaccaccacgcccacgacgccuaccugaacgccguggugggcacggcccugaucaagaaguaccccaagcuggagagcgaguucguguacggcgacuacaagguguacgacgugcggaagaugaucgccaagagcgagcaggagaucggcaaggccacggccaaguacuucuucuacagcaacaucaugaacuucuucaagacggagaucacgcuggccaacggcgagauccggaagcggccccugaucgagacgaacggcgagacgggcgagaucgugugggacaagggccgggacuucgccacggugcggaaggugcugagcaugccccaggugaacaucgugaagaagacggaggugcagacgggcggcuucagcaaggagagcauccugcccaagcggaacagcgacaagcugaucgcccggaagaaggacugggaccccaagaaguacggcggcuucgacagccccacgguggccuacagcgugcuggugguggccaagguggagaagggcaagagcaagaagcugaagagcgugaaggagcugcugggcaucacgaucauggagcggagcagcuucgagaagaaccccaucgacuuccuggaggccaagggcuacaaggaggugaagaaggaccugaucaucaagcugcccaaguacagccuguucgagcuggagaacggccggaagcggaugcuggccagcgccggcgagcugcagaagggcaacgagcuggcccugcccagcaaguacgugaacuuccuguaccuggccagccacuacgagaagcugaagggcagccccgaggacaacgagcagaagcagcuguucguggagcagcacaagcacuaccuggacgagaucaucgagcagaucagcgaguucagcaagcgggugauccuggccgacgccaaccuggacaaggugcugagcgccuacaacaagcaccgggacaagcccauccgggagcaggccgagaacaucauccaccuguucacgcugacgaaccugggcgcccccgccgccuucaaguacuucgacacgacgaucgaccggaagcgguacacgagcacgaaggaggugcuggacgccacgcugauccaccagagcaucacgggccuguacgagacgcggaucgaccugagccagcugggcggcgacggcggcggcagccccaagaagaagcggaagguguag

SEQ ID NO: 6 - RNA, 4140 nt, Artificial Sequence
  Synthetic: E-pair enriched, I-pair depleted Cas9 ORF
auggacaagaaguacagcaucggccuggacaucggcacgaacagcguuggcugggcugugaucacggacgaguacaagguucccagcaagaaguucaaggugcugggcaacacggaccggcacagcaucaagaagaaucugaucggugcacugcuguucgacagcggugagacggccgaagccacgcggcugaagcggacggcccggcggcgguacacgcggcggaagaaccggaucugcuaccugcaggagaucuucagcaacgagauggccaagguggacgacagcuucuuccaccggcuggaggagagcuuccugguggaggaggacaagaagcacgagcggcaccccaucuucggcaacaucguggacgaaguggccuaccacgagaaguaccccacgaucuaccaccugcggaagaagcugguggacucgacggacaaggccgaccugcggcugaucuaccuggcacuggcccacaugaucaaguuccggggccacuuccugaucgagggcgaccugaacccugacaacagcgacguggacaagcuguucauccagcuggugcagaccuacaaccagcuguucgaggagaaccccaucaacgccagcggcguggacgccaaggccauccucagcgcccggcucagcaagagccggcggcuggagaaucugaucgcccagcuucccggugagaagaagaauggccuguucggcaaucugaucgcacucagccugggccugacucccaacuucaagagcaacuucgaccuggccgaggacgccaagcugcagcucagcaaggacaccuacgacgacgaccuggacaaucugcuggcccagaucggcgaccaguacgccgaccuguuccuggcugccaagaaucucagcgacgccauccugcucagcgacauccugcgggugaacacagagaucacgaaggccccccucagcgccagcaugaucaagcgguacgacgagcaccaccaggaccugacgcugcugaaggcacuggugcggcagcagcuuccagagaaguacaaggagaucuucuucgaccagagcaagaauggcuacgccggcuacaucgacgguggugccagccaggaggaguucuacaaguucaucaagcccauccuggagaagauggacggcacagaggagcugcuggugaagcugaacagggaggaccugcugcggaagcagcggacguucgacaauggcagcaucccccaccagauccaccugggugagcugcacgccauccugcggcggcaggaggacuucuaccccuuccugaaggacaacagggagaagaucgagaagauccugacguuccggauccccuacuacguuggcccccuggcccggggcaacagccgguucgccuggaugacgcggaagagcgaggagacgaucacucccuggaacuucgaggaagugguggacaagggugccagcgcccagagcuucaucgagcggaugacgaacuucgacaagaaucuucccaacgagaaggugcuucccaagcacagccugcuguacgaguacuucacgguguacaacgagcugacgaaggugaaguacgugacagagggcaugcggaagcccgccuuccucagcggugagcagaagaaggccaucguggaccugcuguucaagacgaaccggaaggugacggugaagcagcugaaggaggacuacuucaagaagaucgagugcuucgacagcguggagaucagcggcguggaggaccgguucaacgccagccugggcaccuaccacgaccugcugaagaucaucaaggacaaggacuuccuggacaacgaggagaacgaggacauccuggaggacaucgugcugacgcugacgcuguucgaggacagggagaugaucgaggagcggcugaagaccuacgcccaccuguucgacgacaaggugaugaagcagcugaagcggcggcgguacacgggcuggggccggcucagccggaagcugaucaauggcauccgagacaagcagagcggcaagacgauccuggacuuccugaagagcgacggcuucgccaaccggaacuucaugcagcugauccacgacgacagccugacguucaaggaggacauccagaaggcccaggucagcggccagggcgacagccugcacgagcacaucgccaaucuggccggcagccccgccaucaagaagggcauccugcagacggugaaggugguggacgagcuggugaaggugaugggccggcacaagccagagaacaucgugaucgagauggccagggagaaccagacgacucagaagggccagaagaacagcagggagcggaugaagcggaucgaggagggcaucaaggagcugggcagccagauccugaaggagcaccccguggagaacacucagcugcagaacgagaagcuguaccuguacuaccugcagaauggccgagacauguacguggaccaggagcuggacaucaaccggcucagcgacuacgacguggaccacaucguuccccagagcuuccugaaggacgacagcaucgacaacaaggugcugacgcggagcgacaagaaccggggcaagagcgacaacguucccagcgaggaaguggugaagaagaugaagaacuacuggcggcagcugcugaacgccaagcugaucacucagcggaaguucgacaaucugacgaaggccgagcgggguggccucagcgagcuggacaaggccggcuucaucaagcggcagcugguggagacgcggcagaucacgaagcacguggcccagauccuggacagccggaugaacacgaaguacgacgagaacgacaagcugaucagggaagugaaggugaucacgcugaagagcaagcuggucagcgacuuccggaaggacuuccaguucuacaaggugagggagaucaacaacuaccaccacgcccacgacgccuaccugaacgcugugguuggcacggcacugaucaagaaguaccccaagcuggagagcgaguucguguacggcgacuacaagguguacgacgugcggaagaugaucgccaagagcgagcaggagaucggcaaggccacggccaaguacuucuucuacagcaacaucaugaacuucuucaagacagagaucacgcuggccaauggugagauccggaagcggccccugaucgagacgaauggugagacgggugagaucgugugggacaagggccgagacuucgccacggugcggaaggugcucagcaugccccaggugaacaucgugaagaagacagaagugcagacggguggcuucagcaaggagagcauccuucccaagcggaacagcgacaagcugaucgcccggaagaaggacugggaccccaagaaguacgguggcuucgacagccccacgguggccuacagcgugcuggugguggccaagguggagaagggcaagagcaagaagcugaagagcgugaaggagcugcugggcaucacgaucauggagcggagcagcuucgagaagaaccccaucgacuuccuggaagccaagggcuacaaggaagugaagaaggaccugaucaucaagcuucccaaguacagccuguucgagcuggagaauggccggaagcggaugcuggccagcgccggugagcugcagaagggcaacgagcuggcacuucccagcaaguacgugaacuuccuguaccuggccagccacuacgagaagcugaagggcagcccagaggacaacgagcagaagcagcuguucguggagcagcacaagcacuaccuggacgagaucaucgagcagaucagcgaguucagcaagcgggugauccuggccgacgccaaucuggacaaggugcucagcgccuacaacaagcaccgagacaagcccaucagggagcaggccgagaacaucauccaccuguucacgcugacgaaucugggugcccccgcugccuucaaguacuucgacacgacgaucgaccggaagcgguacacgucgacgaaggaagugcuggacgccacgcugauccaccagagcaucacgggccuguacgagacgcggaucgaccucagccagcuggguggcgacggugguggcagccccaagaagaagcggaagguguag

SEQ ID NO: 7 - RNA, 4140 nt, Artificial Sequence
  Synthetic: E-pair & E-single enriched, I-pair & I-single depleted Cas9 ORF
auggacaagaaguacagcaucggccucgacaucggcaccaacagcguuggcugggcugugaucaccgacgaguacaagguucccucaaagaaguucaagguccucggcaacaccgaccgccacagcaucaagaagaaucucaucggugcacugcucuucgacagcggugagaccgcggaagccacccgccucaagcggaccgcccgccgccgcuacacccgccgcaagaaccgcaucugcuaccuccaggagaucuucagcaacgagauggccaaggucgacgacagcuucuuccaccgccucgaggagagcuuccuggucgaggaggacaagaagcacgagcgccaccccaucuucggcaacaucgucgacgaagucgccuaccacgagaaguaccccaccaucuaccaccugcggaagaagcucgucgacucgacugacaaggccgaccugcggcucaucuaccucgcacuggcccacaugauaaaguuccgcggccacuuccugaucgagggcgaccucaacccugacaacagcgacgucgacaagcucuucauccagcucguccagaccuacaaccagcucuucgaggagaaccccaucaacgccagcggcgucgacgccaaggccauccucagcgcccgccucagcaagagccgccgccucgagaaucucaucgcccagcuuccaggugagaagaagaaugggcucuucggcaaucucaucgcacucagccucggccucacucccaacuucaagagcaacuucgaccucgcggaggacgccaagcuccagcucagcaaggacaccuacgacgacgaccucgacaaucuccucgcccagaucggcgaccaguacgccgaccucuuccuggcugccaagaaucucagcgacgccauccuccucagcgacauccugcgggucaacacagagaucaccaaggccccccucagcgccagcaugauaaagcgcuacgacgagcaccaccaggaccucacccuccucaaggcacugguccgccagcagcuuccagagaaguacaaggagaucuucuucgaccagagcaagaauggguacgccggguacaucgacgguggugccagccaggaggaguucuacaaguucaucaagcccauccucgagaagauggacggcacagaggagcugcucgucaagcucaacagggaggaccuccugcggaagcagcggaccuucgacaaugggagcaucccccaccagauccaccucggugagcugcacgccauccugcggcgccaggaggacuucuaccccuuccugaaggacaacagggagaagaucgagaagauccucaccuuccgcauccccuacuacguuggcccccucgcccgcggcaacagccgcuucgccuggaugacccgcaagagcgaggagaccaucacucccuggaacuucgaggaagucgucgacaagggugccagcgcccagagcuucaucgagcgcaugaccaacuucgacaagaaucuuccaaacgagaagguccuuccaaagcacagccuccucuacgaguacuucaccgucuacaacgagcugaccaaggucaaguacgucacagagggcaugcgcaagccagccuuccucagcggugagcagaagaaggccaucgucgaccuccucuucaagaccaaccgcaaggucaccgucaagcagcucaaggaggacuacuucaagaagaucgagugcuucgacagcgucgagaucagcggcgucgaggaccgcuucaacgccagccucggcaccuaccacgaccuccucaagaucaucaaggacaaggacuuccuggacaacgaggagaacgaggacauccucgaggacaucguccucacccucacccucuucgaggacagggagaugauagaggagcgccucaagaccuacgcccaccucuucgacgacaaggucaugaagcagcucaagcgccgccgcuacaccggcuggggccgccucagccgcaagcucaucaaugggauccgagacaagcagagcggcaagaccauccucgacuuccugaagagcgacggcuucgccaaccgcaacuucaugcagcucauccacgacgacagccucaccuucaaggaggacauccagaaggcccaggucagcggccagggcgacagccuccacgagcacaucgccaaucucgccgggagcccagccaucaagaaggggauccuccagaccgucaaggucgucgacgagcuggucaaggucaugggccgccacaagccagagaacaucgucaucgagauggccagggagaaccagaccacucaaaaggggcagaagaacagcagggagcgcaugaagcgcaucgaggagggcaucaaggagcugggcagccagauccucaaggagcaccccgucgagaacacucaacuccagaacgagaagcucuaccucuacuaccuccagaaugggcgagacauguacgucgaccaggagcuggacaucaaccgccucagcgacuacgacgucgaccacaucguuccccagagcuuccugaaggacgacagcaucgacaacaagguccucacccgaagcgacaagaaccgcggcaagagcgacaacguucccucagaggaagucgucaagaagaugaagaacuacuggcgccagcuccucaacgccaagcucaucacucaacgcaaguucgacaaucucaccaaggcggagcgcgguggccucagcgagcuggacaaggccggguucaucaagcgccagcucgucgagacccgccagaucaccaagcacgucgcccagauccucgacagccgcaugaacaccaaguacgacgagaacgacaagcucaucagggaagucaaggucaucacccucaagagcaagcucgucagcgacuuccgcaaggacuuccaguucuacaaggucagggagaucaacaacuaccaccacgcccacgacgccuaccucaacgcugugguuggcaccgcacugaucaagaaguaccccaagcucgagagcgaguucgucuacggcgacuacaaggucuacgacguccgcaagaugauagccaagagcgagcaggagaucggcaaggccaccgccaaguacuucuucuacagcaacaucaugaacuucuucaagacagagaucacccucgccaauggugagauccgcaagcgcccccucaucgagaccaauggugagaccggugagaucgucugggacaaggggcgagacuucgccaccguccgcaagguccucagcaugccccaggugaacaucgucaagaagacagaaguccagaccgguggcuucagcaaggagagcauccuuccaaagcgcaacagcgacaagcucaucgcccgcaagaaggacugggaccccaagaaguacgguggcuucgacagccccaccgucgccuacagcguccucgucgucgccaaggucgagaaggggaagagcaagaagcucaagagcgucaaggagcugcucggcaucaccaucauggagcgaagcagcuucgagaagaaccccaucgacuuccuggaagccaagggguacaaggaagucaagaaggaccucaucaucaagcuuccaaaguacagccucuucgagcuggagaaugggcgcaagcgcaugcucgccagcgccggugagcugcagaaggggaacgagcuggcacuucccucaaaguacgucaacuuccuguaccucgccagccacuacgagaagcucaaggggagcccagaggacaacgagcagaagcagcucuucgucgagcagcacaagcacuaccucgacgagaucaucgagcagaucagcgaguucagcaagcgcgucauccucgccgacgccaaucucgacaagguccucagcgccuacaacaagcaccgagacaagcccaucagggagcaggccgagaacaucauccaccucuucacccucaccaaucucggugccccagcugccuucaaguacuucgacaccaccaucgaccgcaagcgcuacaccucgacuaaggaaguccucgacgccacccucauccaccagagcaucaccggccucuacgagacccgcaucgaccucagccagcucgguggcgacggugguggcagccccaagaagaagcgcaaggucuag

SEQ ID NO: 8 - RNA, 4140 nt, Artificial Sequence
  Synthetic: I-pair depleted and/or I-single depleted Cas9 ORF
auggacaagaaguacagcaucggccuggacaucggcacgaacagcguuggcugggcugugaucacggacgaguacaagguucccucaaagaaguucaaggugcugggcaacacggaccggcacagcaucaagaagaaucucaucggugcacugcuguucgacagcggugagacggccgaagccacgcggcugaagcggacggcccgccggcgguacacgcggcggaagaaccggaucugcuaccugcaggagaucuucagcaacgagauggccaagguggacgacagcuucuuccaccggcuggaggagagcuuccugguggaggaggacaagaagcacgagcggcaccccaucuucggcaacaucguggacgaagucgccuaccacgagaaguaccccaccaucuaccaccugcggaagaagcugguggacucgacugacaaggccgaccugcggcugaucuaccuggcacuggcccacaugauaaaguuccggggccacuuccugaucgagggcgaccugaacccugacaacagcgacguggacaagcuguucauccagcuggugcagaccuacaaccagcuguucgaggagaaccccaucaacgccagcggcguggacgccaaggccauccucagcgcccgccucagcaagagccggcggcuggagaaucucaucgcccagcuuccaggugagaagaagaaugggcuguucggcaaucucaucgcacucagccugggccugacucccaacuucaagagcaacuucgaccuggccgaggacgccaagcugcagcucagcaaggacaccuacgacgacgaccuggacaaucuccuggcccagaucggcgaccaguacgccgaccuguuccuggcugccaagaaucucagcgacgccauccugcucagcgacauccugcgggugaacacagagaucacgaaggccccccucagcgccagcaugauaaagcgguacgacgagcaccaccaggaccugacgcugcugaaggcacuggugcggcagcagcuuccagagaaguacaaggagaucuucuucgaccagagcaagaauggguacgccggguacaucgacgguggugccagccaggaggaguucuacaaguucaucaagcccauccuggagaagauggacggcacagaggagcugcuggugaagcugaacagggaggaccugcugcggaagcagcggacguucgacaaugggagcaucccccaccagauccaccugggugagcugcacgccauccugcggcggcaggaggacuucuaccccuuccugaaggacaacagggagaagaucgagaagauccugacguuccggauccccuacuacguuggcccccuggcccgcggcaacagccgguucgccuggaugacgcggaagagcgaggagacgaucacucccuggaacuucgaggaagucguggacaagggugccagcgcccagagcuucaucgagcggaugacgaacuucgacaagaaucuuccaaacgagaaggugcuuccaaagcacagccugcuguacgaguacuucacgguguacaacgagcugacgaaggugaaguacgugacagagggcaugcggaagcccgccuuccucagcggugagcagaagaaggccaucguggaccugcuguucaagacgaaccggaaggugacggugaagcagcugaaggaggacuacuucaagaagaucgagugcuucgacagcguggagaucagcggcguggaggaccgguucaacgccagccugggcaccuaccacgaccugcugaagaucaucaaggacaaggacuuccuggacaacgaggagaacgaggacauccuggaggacaucgugcugacgcugacgcuguucgaggacagggagaugauagaggagcggcugaagaccuacgcccaccuguucgacgacaaggugaugaagcagcugaagcggcggcgguacacgggcuggggccggcucagccggaagcugaucaaugggauccgagacaagcagagcggcaagacgauccuggacuuccugaagagcgacggcuucgccaaccggaacuucaugcagcugauccacgacgacagccugacguucaaggaggacauccagaaggcccaggucagcggccagggcgacagccugcacgagcacaucgccaaucucgccgggagccccgccaucaagaaggggauccugcagacggugaaggugguggacgagcuggugaaggugaugggccggcacaagccagagaacaucgugaucgagauggccagggagaaccagacgacucaaaaggggcagaagaacagcagggagcggaugaagcggaucgaggagggcaucaaggagcugggcagccagauccugaaggagcaccccguggagaacacucaacugcagaacgagaagcuguaccuguacuaccugcagaaugggcgagacauguacguggaccaggagcuggacaucaaccggcucagcgacuacgacguggaccacaucguuccccagagcuuccugaaggacgacagcaucgacaacaaggugcugacgcggagcgacaagaaccggggcaagagcgacaacguucccucagaggaagucgugaagaagaugaagaacuacuggcggcagcugcugaacgccaagcugaucacucaacggaaguucgacaaucucacgaaggccgagcgggguggccucagcgagcuggacaaggccggguucaucaagcggcagcugguggagacgcggcagaucacgaagcacguggcccagauccuggacagccggaugaacacgaaguacgacgagaacgacaagcugaucagggaagucaaggugaucacgcugaagagcaagcuggucagcgacuuccggaaggacuuccaguucuacaaggugagggagaucaacaacuaccaccacgcccacgacgccuaccugaacgcugugguuggcacggcacugaucaagaaguaccccaagcuggagagcgaguucguguacggcgacuacaagguguacgacgugcggaagaugauagccaagagcgagcaggagaucggcaaggccacggccaaguacuucuucuacagcaacaucaugaacuucuucaagacagagaucacgcuggccaauggugagauccggaagcggccccugaucgagacgaauggugagacgggugagaucgugugggacaaggggcgagacuucgccacggugcggaaggugcucagcaugccccaggugaacaucgugaagaagacagaaguccagacggguggcuucagcaaggagagcauccuuccaaagcggaacagcgacaagcugaucgcccgcaagaaggacugggaccccaagaaguacgguggcuucgacagccccaccguggccuacagcgugcuggugguggccaagguggagaaggggaagagcaagaagcugaagagcgugaaggagcugcugggcaucacgaucauggagcggagcagcuucgagaagaaccccaucgacuuccuggaagccaagggguacaaggaagucaagaaggaccugaucaucaagcuuccaaaguacagccuguucgagcuggagaaugggcggaagcggaugcuggccagcgccggugagcugcagaaggggaacgagcuggcacuucccucaaaguacgugaacuuccuguaccuggccagccacuacgagaagcugaaggggagcccagaggacaacgagcagaagcagcuguucguggagcagcacaagcacuaccuggacgagaucaucgagcagaucagcgaguucagcaagcgggugauccuggccgacgccaaucucgacaaggugcucagcgccuacaacaagcaccgagacaagcccaucagggagcaggccgagaacaucauccaccuguucacgcugacgaaucucggugcccccgcugccuucaaguacuucgacacgacgaucgaccggaagcgguacacgucgacuaaggaaguccuggacgccacgcugauccaccagagcaucacgggccuguacgagacgcggaucgaccucagccagcuggguggcgacggugguggcagccccaagaagaagcggaagguguag

SEQ ID NO: 9 - RNA, 4140 nt, Artificial Sequence
  Synthetic: E-Pair enriched Cas9 ORF
auggacaagaaguacagcaucggccucgacaucggcaccaacagcguuggcugggcugugaucaccgacgaguacaagguucccucaaagaaguucaagguccucggcaacaccgaccgccacagcaucaagaagaaucucaucggugcacugcucuucgacagcggugagaccgcggaagccacccgccucaagcgcaccgcccgccgccgcuacacccgccgcaagaaccgcaucugcuaccuccaggagaucuucagcaacgagauggccaaggucgacgacagcuucuuccaccgccucgaggagagcuuccuggucgaggaggacaagaagcacgagcgccaccccaucuucggcaacaucgucgacgaagucgccuaccacgagaaguaccccaccaucuaccaccugcggaagaagcucgucgacucgacugacaaggccgaccugcggcucaucuaccucgcacuggcccacaugauaaaguuccgcggccacuuccugaucgagggcgaccucaacccugacaacagcgacgucgacaagcucuucauccagcucguccagaccuacaaccagcucuucgaggagaaccccaucaacgccagcggcgucgacgccaaggccauccucagcgcccgccucagcaagagccgccgccucgagaaucucaucgcccagcuuccaggugagaagaagaaugggcucuucggcaaucucaucgcacucagccucggccucacucccaacuucaagagcaacuucgaccucgcggaggacgccaagcuccagcucagcaaggacaccuacgacgacgaccucgacaaucuccucgcccagaucggcgaccaguacgccgaccucuuccuggcugccaagaaucucagcgacgccauccuccucagcgacauccugcgggucaacacagagaucaccaaggccccccucagcgccagcaugauaaagcgcuacgacgagcaccaccaggaccucacccuccucaaggcacugguccgccagcagcuuccagagaaguacaaggagaucuucuucgaccagagcaagaauggguacgccggguacaucgacgguggugccagccaggaggaguucuacaaguucaucaagcccauccucgagaagauggacggcacagaggagcugcucgucaagcucaacagggaggaccuccugcggaagcagcgcaccuucgacaaugggagcaucccccaccagauccaccucggugagcugcacgccauccugcggcgccaggaggacuucuaccccuuccugaaggacaacagggagaagaucgagaagauccucaccuuccgcauccccuacuacguuggcccccucgcccgcggcaacagccgcuucgccuggaugacccgcaagagcgaggagaccaucacucccuggaacuucgaggaagucgucgacaagggugccagcgcccagagcuucaucgagcgcaugaccaacuucgacaagaaucuuccaaacgagaagguccuuccaaagcacagccuccucuacgaguacuucaccgucuacaacgagcugaccaaggucaaguacgucacagagggcaugcgcaagccagccuuccucagcggugagcagaagaaggccaucgucgaccuccucuucaagaccaaccgcaaggucaccgucaagcagcucaaggaggacuacuucaagaagaucgagugcuucgacagcgucgagaucagcggcgucgaggaccgcuucaacgccagccucggcaccuaccacgaccuccucaagaucaucaaggacaaggacuuccuggacaacgaggagaacgaggacauccucgaggacaucguccucacccucacccucuucgaggacagggagaugauagaggagcgccucaagaccuacgcccaccucuucgacgacaaggucaugaagcagcucaagcgccgccgcuacaccggcuggggccgccucagccgcaagcucaucaaugggauccgagacaagcagagcggcaagaccauccucgacuuccugaagagcgacggcuucgccaaccgcaacuucaugcagcucauccacgacgacagccucaccuucaaggaggacauccagaaggcccaggucagcggccagggcgacagccuccacgagcacaucgccaaucucgccgggagcccagccaucaagaaggggauccuccagaccgucaaggucgucgacgagcuggucaaggucaugggccgccacaagccagagaacaucgucaucgagauggccagggagaaccagaccacucaaaaggggcagaagaacagcagggagcgcaugaagcgcaucgaggagggcaucaaggagcugggcagccagauccucaaggagcaccccgucgagaacacucaacuccagaacgagaagcucuaccucuacuaccuccagaaugggcgagacauguacgucgaccaggagcuggacaucaaccgccucagcgacuacgacgucgaccacaucguuccccagagcuuccugaaggacgacagcaucgacaacaagguccucacccgaagcgacaagaaccgcggcaagagcgacaacguucccucagaggaagucgucaagaagaugaagaacuacuggcgccagcuccucaacgccaagcucaucacucaacgcaaguucgacaaucucaccaaggcggagcgcgguggccucagcgagcuggacaaggccggguucaucaagcgccagcucgucgagacccgccagaucaccaagcacgucgcccagauccucgacagccgcaugaacaccaaguacgacgagaacgacaagcucaucagggaagucaaggucaucacccucaagagcaagcucgucagcgacuuccgcaaggacuuccaguucuacaaggucagggagaucaacaacuaccaccacgcccacgacgccuaccucaacgcugugguuggcaccgcacugaucaagaaguaccccaagcucgagagcgaguucgucuacggcgacuacaaggucuacgacguccgcaagaugauagccaagagcgagcaggagaucggcaaggccaccgccaaguacuucuucuacagcaacaucaugaacuucuucaagacagagaucacccucgccaauggugagauccgcaagcgcccccucaucgagaccaauggugagaccggugagaucgucugggacaaggggcgagacuucgccaccguccgcaagguccucagcaugccccaggugaacaucgucaagaagacagaaguccagaccgguggcuucagcaaggagagcauccuuccaaagcgcaacagcgacaagcucaucgcccgcaagaaggacugggaccccaagaaguacgguggcuucgacagccccaccgucgccuacagcguccucgucgucgccaaggucgagaaggggaagagcaagaagcucaagagcgucaaggagcugcucggcaucaccaucauggagcgaagcagcuucgagaagaaccccaucgacuuccuggaagccaagggguacaaggaagucaagaaggaccucaucaucaagcuuccaaaguacagccucuucgagcuggagaaugggcgcaagcgcaugcucgccagcgccggugagcugcagaaggggaacgagcuggcacuucccucaaaguacgucaacuuccuguaccucgccagccacuacgagaagcucaaggggagcccagaggacaacgagcagaagcagcucuucgucgagcagcacaagcacuaccucgacgagaucaucgagcagaucagcgaguucagcaagcgcgucauccucgccgacgccaaucucgacaagguccucagcgccuacaacaagcaccgagacaagcccaucagggagcaggccgagaacaucauccaccucuucacccucaccaaucucggugccccagcugccuucaaguacuucgacaccaccaucgaccgcaagcgcuacaccucgacuaaggaaguccucgacgccacccucauccaccagagcaucaccggccucuacgagacccgcaucgaccucagccagcucgguggcgacggugguggcagccccaagaagaagcgcaaggucuag

SEQ ID NO: 10 - RNA, 4140 nt, Artificial Sequence
  Synthetic: E-pair and E-single enriched Cas9 ORF
auggacaagaaguacagcaucggccucgacaucggcaccaacagcgucggcugggccgucaucaccgacgaguacaagguccccagcaagaaguucaagguccucggcaacaccgaccgccacagcaucaagaagaaccucaucggcgcccuccucuucgacagcggcgagaccgccgaggccacccgccucaagcgcaccgcccgccgccgcuacacccgccgcaagaaccgcaucugcuaccuccaggagaucuucagcaacgagauggccaaggucgacgacagcuucuuccaccgccucgaggagagcuuccucgucgaggaggacaagaagcacgagcgccaccccaucuucggcaacaucgucgacgaggucgccuaccacgagaaguaccccaccaucuaccaccuccgcaagaagcucgucgacagcaccgacaaggccgaccuccgccucaucuaccucgcccucgcccacaugaucaaguuccgcggccacuuccucaucgagggcgaccucaaccccgacaacagcgacgucgacaagcucuucauccagcucguccagaccuacaaccagcucuucgaggagaaccccaucaacgccagcggcgucgacgccaaggccauccucagcgcccgccucagcaagagccgccgccucgagaaccucaucgcccagcuccccggcgagaagaagaacggccucuucggcaaccucaucgcccucagccucggccucacccccaacuucaagagcaacuucgaccucgccgaggacgccaagcuccagcucagcaaggacaccuacgacgacgaccucgacaaccuccucgcccagaucggcgaccaguacgccgaccucuuccucgccgccaagaaccucagcgacgccauccuccucagcgacauccuccgcgucaacaccgagaucaccaaggccccccucagcgccagcaugaucaagcgcuacgacgagcaccaccaggaccucacccuccucaaggcccucguccgccagcagcuccccgagaaguacaaggagaucuucuucgaccagagcaagaacggcuacgccggcuacaucgacggcggcgccagccaggaggaguucuacaaguucaucaagcccauccucgagaagauggacggcaccgaggagcuccucgucaagcucaaccgcgaggaccuccuccgcaagcagcgcaccuucgacaacggcagcaucccccaccagauccaccucggcgagcuccacgccauccuccgccgccaggaggacuucuaccccuuccucaaggacaaccgcgagaagaucgagaagauccucaccuuccgcauccccuacuacgucggcccccucgcccgcggcaacagccgcuucgccuggaugacccgcaagagcgaggagaccaucacccccuggaacuucgaggaggucgucgacaagggcgccagcgcccagagcuucaucgagcgcaugaccaacuucgacaagaaccuccccaacgagaagguccuccccaagcacagccuccucuacgaguacuucaccgucuacaacgagcucaccaaggucaaguacgucaccgagggcaugcgcaagcccgccuuccucagcggcgagcagaagaaggccaucgucgaccuccucuucaagaccaaccgcaaggucaccgucaagcagcucaaggaggacuacuucaagaagaucgagugcuucgacagcgucgagaucagcggcgucgaggaccgcuucaacgccagccucggcaccuaccacgaccuccucaagaucaucaaggacaaggacuuccucgacaacgaggagaacgaggacauccucgaggacaucguccucacccucacccucuucgaggaccgcgagaugaucgaggagcgccucaagaccuacgcccaccucuucgacgacaaggucaugaagcagcucaagcgccgccgcuacaccggcuggggccgccucagccgcaagcucaucaacggcauccgcgacaagcagagcggcaagaccauccucgacuuccucaagagcgacggcuucgccaaccgcaacuucaugcagcucauccacgacgacagccucaccuucaaggaggacauccagaaggcccaggucagcggccagggcgacagccuccacgagcacaucgccaaccucgccggcagccccgccaucaagaagggcauccuccagaccgucaaggucgucgacgagcucgucaaggucaugggccgccacaagcccgagaacaucgucaucgagauggcccgcgagaaccagaccacccagaagggccagaagaacagccgcgagcgcaugaagcgcaucgaggagggcaucaaggagcucggcagccagauccucaaggagcaccccgucgagaacacccagcuccagaacgagaagcucuaccucuacuaccuccagaacggccgcgacauguacgucgaccaggagcucgacaucaaccgccucagcgacuacgacgucgaccacaucgucccccagagcuuccucaaggacgacagcaucgacaacaagguccucacccgcagcgacaagaaccgcggcaagagcgacaacguccccagcgaggaggucgucaagaagaugaagaacuacuggcgccagcuccucaacgccaagcucaucacccagcgcaaguucgacaaccucaccaaggccgagcgcggcggccucagcgagcucgacaaggccggcuucaucaagcgccagcucgucgagacccgccagaucaccaagcacgucgcccagauccucgacagccgcaugaacaccaaguacgacgagaacgacaagcucauccgcgaggucaaggucaucacccucaagagcaagcucgucagcgacuuccgcaaggacuuccaguucuacaagguccgcgagaucaacaacuaccaccacgcccacgacgccuaccucaacgccgucgucggcaccgcccucaucaagaaguaccccaagcucgagagcgaguucgucuacggcgacuacaaggucuacgacguccgcaagaugaucgccaagagcgagcaggagaucggcaaggccaccgccaaguacuucuucuacagcaacaucaugaacuucuucaagaccgagaucacccucgccaacggcgagauccgcaagcgcccccucaucgagaccaacggcgagaccggcgagaucgucugggacaagggccgcgacuucgccaccguccgcaagguccucagcaugccccaggucaacaucgucaagaagaccgagguccagaccggcggcuucagcaaggagagcauccuccccaagcgcaacagcgacaagcucaucgcccgcaagaaggacugggaccccaagaaguacggcggcuucgacagccccaccgucgccuacagcguccucgucgucgccaaggucgagaagggcaagagcaagaagcucaagagcgucaaggagcuccucggcaucaccaucauggagcgcagcagcuucgagaagaaccccaucgacuuccucgaggccaagggcuacaaggaggucaagaaggaccucaucaucaagcuccccaaguacagccucuucgagcucgagaacggccgcaagcgcaugcucgccagcgccggcgagcuccagaagggcaacgagcucgcccuccccagcaaguacgucaacuuccucuaccucgccagccacuacgagaagcucaagggcagccccgaggacaacgagcagaagcagcucuucgucgagcagcacaagcacuaccucgacgagaucaucgagcagaucagcgaguucagcaagcgcgucauccucgccgacgccaaccucgacaagguccucagcgccuacaacaagcaccgcgacaagcccauccgcgagcaggccgagaacaucauccaccucuucacccucaccaaccucggcgcccccgccgccuucaaguacuucgacaccaccaucgaccgcaagcgcuacaccagcaccaaggagguccucgacgccacccucauccaccagagcaucaccggccucuacgagacccgcaucgaccucagccagcucggcggcgacggcggcggcagccccaagaagaagcgcaaggucuag

SEQ ID NO: 11 - RNA, 4140 nt, Artificial Sequence
  Synthetic: E-single depleted Cas9 ORF
auggacaaaaaauacucaauaggccucgacauaggcaccaacucagucggcugggccgucauaaccgacgaguacaaaguccccucaaaaaaauucaaaguccucggcaacaccgacaggcacucaauaaaaaaaaaccucauaggcgcccuccucuucgacucaggcgagaccgccgaggccaccaggcucaaaaggaccgccaggaggagguacaccaggaggaaaaacaggauaugcuaccuccaggagauauucucaaacgagauggccaaagucgacgacucauucuuccacaggcucgaggagucauuccucgucgaggaggacaaaaaacacgagaggcaccccauauucggcaacauagucgacgaggucgccuaccacgagaaauaccccaccauauaccaccucaggaaaaaacucgucgacucaaccgacaaagccgaccucaggcucauauaccucgcccucgcccacaugauaaaauucaggggccacuuccucauagagggcgaccucaaccccgacaacucagacgucgacaaacucuucauacagcucguccagaccuacaaccagcucuucgaggagaaccccauaaacgccucaggcgucgacgccaaagccauacucucagccaggcucucaaaaucaaggaggcucgagaaccucauagcccagcuccccggcgagaaaaaaaacggccucuucggcaaccucauagcccucucacucggccucacccccaacuucaaaucaaacuucgaccucgccgaggacgccaaacuccagcucucaaaagacaccuacgacgacgaccucgacaaccuccucgcccagauaggcgaccaguacgccgaccucuuccucgccgccaaaaaccucucagacgccauacuccucucagacauacucagggucaacaccgagauaaccaaagccccccucucagccucaaugauaaaaagguacgacgagcaccaccaggaccucacccuccucaaagcccucgucaggcagcagcuccccgagaaauacaaagagauauucuucgaccagucaaaaaacggcuacgccggcuacauagacggcggcgccucacaggaggaguucuacaaauucauaaaacccauacucgagaaaauggacggcaccgaggagcuccucgucaaacucaacagggaggaccuccucaggaaacagaggaccuucgacaacggcucaauaccccaccagauacaccucggcgagcuccacgccauacucaggaggcaggaggacuucuaccccuuccucaaagacaacagggagaaaauagagaaaauacucaccuucaggauacccuacuacgucggcccccucgccaggggcaacucaagguucgccuggaugaccaggaaaucagaggagaccauaacccccuggaacuucgaggaggucgucgacaaaggcgccucagcccagucauucauagagaggaugaccaacuucgacaaaaaccuccccaacgagaaaguccuccccaaacacucacuccucuacgaguacuucaccgucuacaacgagcucaccaaagucaaauacgucaccgagggcaugaggaaacccgccuuccucucaggcgagcagaaaaaagccauagucgaccuccucuucaaaaccaacaggaaagucaccgucaaacagcucaaagaggacuacuucaaaaaaauagagugcuucgacucagucgagauaucaggcgucgaggacagguucaacgccucacucggcaccuaccacgaccuccucaaaauaauaaaagacaaagacuuccucgacaacgaggagaacgaggacauacucgaggacauaguccucacccucacccucuucgaggacagggagaugauagaggagaggcucaaaaccuacgcccaccucuucgacgacaaagucaugaaacagcucaaaaggaggagguacaccggcuggggcaggcucucaaggaaacucauaaacggcauaagggacaaacagucaggcaaaaccauacucgacuuccucaaaucagacggcuucgccaacaggaacuucaugcagcucauacacgacgacucacucaccuucaaagaggacauacagaaagcccaggucucaggccagggcgacucacuccacgagcacauagccaaccucgccggcucacccgccauaaaaaaaggcauacuccagaccgucaaagucgucgacgagcucgucaaagucaugggcaggcacaaacccgagaacauagucauagagauggccagggagaaccagaccacccagaaaggccagaaaaacucaagggagaggaugaaaaggauagaggagggcauaaaagagcucggcucacagauacucaaagagcaccccgucgagaacacccagcuccagaacgagaaacucuaccucuacuaccuccagaacggcagggacauguacgucgaccaggagcucgacauaaacaggcucucagacuacgacgucgaccacauagucccccagucauuccucaaagacgacucaauagacaacaaaguccucaccaggucagacaaaaacaggggcaaaucagacaacguccccucagaggaggucgucaaaaaaaugaaaaacuacuggaggcagcuccucaacgccaaacucauaacccagaggaaauucgacaaccucaccaaagccgagaggggcggccucucagagcucgacaaagccggcuucauaaaaaggcagcucgucgagaccaggcagauaaccaaacacgucgcccagauacucgacucaaggaugaacaccaaauacgacgagaacgacaaacucauaagggaggucaaagucauaacccucaaaucaaaacucgucucagacuucaggaaagacuuccaguucuacaaagucagggagauaaacaacuaccaccacgcccacgacgccuaccucaacgccgucgucggcaccgcccucauaaaaaaauaccccaaacucgagucagaguucgucuacggcgacuacaaagucuacgacgucaggaaaaugauagccaaaucagagcaggagauaggcaaagccaccgccaaauacuucuucuacucaaacauaaugaacuucuucaaaaccgagauaacccucgccaacggcgagauaaggaaaaggccccucauagagaccaacggcgagaccggcgagauagucugggacaaaggcagggacuucgccaccgucaggaaaguccucucaaugccccaggucaacauagucaaaaaaaccgagguccagaccggcggcuucucaaaagagucaauacuccccaaaaggaacucagacaaacucauagccaggaaaaaagacugggaccccaaaaaauacggcggcuucgacucacccaccgucgccuacucaguccucgucgucgccaaagucgagaaaggcaaaucaaaaaaacucaaaucagucaaagagcuccucggcauaaccauaauggagaggucaucauucgagaaaaaccccauagacuuccucgaggccaaaggcuacaaagaggucaaaaaagaccucauaauaaaacuccccaaauacucacucuucgagcucgagaacggcaggaaaaggaugcucgccucagccggcgagcuccagaaaggcaacgagcucgcccuccccucaaaauacgucaacuuccucuaccucgccucacacuacgagaaacucaaaggcucacccgaggacaacgagcagaaacagcucuucgucgagcagcacaaacacuaccucgacgagauaauagagcagauaucagaguucucaaaaagggucauacucgccgacgccaaccucgacaaaguccucucagccuacaacaaacacagggacaaacccauaagggagcaggccgagaacauaauacaccucuucacccucaccaaccucggcgcccccgccgccuucaaauacuucgacaccaccauagacaggaaaagguacaccucaaccaaagagguccucgacgccacccucauacaccagucaauaaccggccucuacgagaccaggauagaccucucacagcucggcggcgacggcggcggcucacccaaaaaaaaaaggaaagucuag

SEQ ID NO: 12 - RNA, 4140 nt, Artificial Sequence
  Synthetic: I-single enriched Cas9 ORF
auggacaaaaaauacagcaucggccuggacaucggcacgaacagcgugggcugggccgugaucacggacgaguacaaagugcccagcaaaaaauucaaagugcugggcaacacggaccggcacagcaucaaaaaaaaccugaucggcgcccugcuguucgacagcggcgagacggccgaggccacgcggcugaaacggacggcccggcggcgguacacgcggcggaaaaaccggaucugcuaccugcaggagaucuucagcaacgagauggccaaaguggacgacagcuucuuccaccggcuggaggagagcuuccugguggaggaggacaaaaaacacgagcggcaccccaucuucggcaacaucguggacgagguggccuaccacgagaaauaccccacgaucuaccaccugcggaaaaaacugguggacagcacggacaaagccgaccugcggcugaucuaccuggcccuggcccacaugaucaaauuccggggccacuuccugaucgagggcgaccugaaccccgacaacagcgacguggacaaacuguucauccagcuggugcagacguacaaccagcuguucgaggagaaccccaucaacgccagcggcguggacgccaaagccauccugagcgcccggcugagcaaaagccggcggcuggagaaccugaucgcccagcugcccggcgagaaaaaaaacggccuguucggcaaccugaucgcccugagccugggccugacgcccaacuucaaaagcaacuucgaccuggccgaggacgccaaacugcagcugagcaaagacacguacgacgacgaccuggacaaccugcuggcccagaucggcgaccaguacgccgaccuguuccuggccgccaaaaaccugagcgacgccauccugcugagcgacauccugcgggugaacacggagaucacgaaagccccccugagcgccagcaugaucaaacgguacgacgagcaccaccaggaccugacgcugcugaaagcccuggugcggcagcagcugcccgagaaauacaaagagaucuucuucgaccagagcaaaaacggcuacgccggcuacaucgacggcggcgccagccaggaggaguucuacaaauucaucaaacccauccuggagaaaauggacggcacggaggagcugcuggugaaacugaaccgggaggaccugcugcggaaacagcggacguucgacaacggcagcaucccccaccagauccaccugggcgagcugcacgccauccugcggcggcaggaggacuucuaccccuuccugaaagacaaccgggagaaaaucgagaaaauccugacguuccggauccccuacuacgugggcccccuggcccggggcaacagccgguucgccuggaugacgcggaaaagcgaggagacgaucacgcccuggaacuucgaggaggugguggacaaaggcgccagcgcccagagcuucaucgagcggaugacgaacuucgacaaaaaccugcccaacgagaaagugcugcccaaacacagccugcuguacgaguacuucacgguguacaacgagcugacgaaagugaaauacgugacggagggcaugcggaaacccgccuuccugagcggcgagcagaaaaaagccaucguggaccugcuguucaaaacgaaccggaaagugacggugaaacagcugaaagaggacuacuucaaaaaaaucgagugcuucgacagcguggagaucagcggcguggaggaccgguucaacgccagccugggcacguaccacgaccugcugaaaaucaucaaagacaaagacuuccuggacaacgaggagaacgaggacauccuggaggacaucgugcugacgcugacgcuguucgaggaccgggagaugaucgaggagcggcugaaaacguacgcccaccuguucgacgacaaagugaugaaacagcugaaacggcggcgguacacgggcuggggccggcugagccggaaacugaucaacggcauccgggacaaacagagcggcaaaacgauccuggacuuccugaaaagcgacggcuucgccaaccggaacuucaugcagcugauccacgacgacagccugacguucaaagaggacauccagaaagcccaggugagcggccagggcgacagccugcacgagcacaucgccaaccuggccggcagccccgccaucaaaaaaggcauccugcagacggugaaagugguggacgagcuggugaaagugaugggccggcacaaacccgagaacaucgugaucgagauggcccgggagaaccagacgacgcagaaaggccagaaaaacagccgggagcggaugaaacggaucgaggagggcaucaaagagcugggcagccagauccugaaagagcaccccguggagaacacgcagcugcagaacgagaaacuguaccuguacuaccugcagaacggccgggacauguacguggaccaggagcuggacaucaaccggcugagcgacuacgacguggaccacaucgugccccagagcuuccugaaagacgacagcaucgacaacaaagugcugacgcggagcgacaaaaaccggggcaaaagcgacaacgugcccagcgaggagguggugaaaaaaaugaaaaacuacuggcggcagcugcugaacgccaaacugaucacgcagcggaaauucgacaaccugacgaaagccgagcggggcggccugagcgagcuggacaaagccggcuucaucaaacggcagcugguggagacgcggcagaucacgaaacacguggcccagauccuggacagccggaugaacacgaaauacgacgagaacgacaaacugauccgggaggugaaagugaucacgcugaaaagcaaacuggugagcgacuuccggaaagacuuccaguucuacaaagugcgggagaucaacaacuaccaccacgcccacgacgccuaccugaacgccguggugggcacggcccugaucaaaaaauaccccaaacuggagagcgaguucguguacggcgacuacaaaguguacgacgugcggaaaaugaucgccaaaagcgagcaggagaucggcaaagccacggccaaauacuucuucuacagcaacaucaugaacuucuucaaaacggagaucacgcuggccaacggcgagauccggaaacggccccugaucgagacgaacggcgagacgggcgagaucgugugggacaaaggccgggacuucgccacggugcggaaagugcugagcaugccccaggugaacaucgugaaaaaaacggaggugcagacgggcggcuucagcaaagagagcauccugcccaaacggaacagcgacaaacugaucgcccggaaaaaagacugggaccccaaaaaauacggcggcuucgacagccccacgguggccuacagcgugcuggugguggccaaaguggagaaaggcaaaagcaaaaaacugaaaagcgugaaagagcugcugggcaucacgaucauggagcggagcagcuucgagaaaaaccccaucgacuuccuggaggccaaaggcuacaaagaggugaaaaaagaccugaucaucaaacugcccaaauacagccuguucgagcuggagaacggccggaaacggaugcuggccagcgccggcgagcugcagaaaggcaacgagcuggcccugcccagcaaauacgugaacuuccuguaccuggccagccacuacgagaaacugaaaggcagccccgaggacaacgagcagaaacagcuguucguggagcagcacaaacacuaccuggacgagaucaucgagcagaucagcgaguucagcaaacgggugauccuggccgacgccaaccuggacaaagugcugagcgccuacaacaaacaccgggacaaacccauccgggagcaggccgagaacaucauccaccuguucacgcugacgaaccugggcgcccccgccgccuucaaauacuucgacacgacgaucgaccggaaacgguacacgagcacgaaagaggugcuggacgccacgcugauccaccagagcaucacgggccuguacgagacgcggaucgaccugagccagcugggcggcgacggcggcggcagccccaaaaaaaaacggaaaguguag

SEQ ID NO: 13 - RNA, 4140 nt, Artificial Sequence
  Synthetic: E-pair depleted Cas9 ORF
auggauaaaaaauauucaauaggauuagauauaggaacaaauucaguaggaugggcaguaauaacagaugaauauaaaguaccaucaaaaaaauuuaaaguauuaggaaauacagauagacauucaauaaaaaaaaauuuaauaggagcauuauuauuugauucaggagaaacagcagaagcaacaagauuaaaaagaacagcaagaagaagauauacaagaagaaaaaauagaauauguuauuuacaagaaauauuuucaaaugaaauggcaaaaguagaugauucauuuuuucauagauuagaagaaucauuuuuaguagaagaagauaaaaaacaugaaagacauccaauauuuggaaauauaguagaugaaguagcauaucaugaaaaauauccaacaauauaucauuuaagaaaaaaauuaguagauucaacagauaaagcagauuuaagauuaauauauuuagcauuagcacauaugauaaaauuuagaggacauuuuuuaauagaaggagauuuaaauccagauaauucagauguagauaaauuauuuauacaauuaguacaaacauauaaucaauuauuugaagaaaauccaauaaaugcaucaggaguagaugcaaaagcaauauuaucagcaagauuaucaaaaucaagaagauuagaaaauuuaauagcacaauuaccaggagaaaaaaaaaauggauuauuuggaaauuuaauagcauuaucauuaggauuaacaccaaauuuuaaaucaaauuuugauuuagcagaagaugcaaaauuacaauuaucaaaagauacauaugaugaugauuuagauaauuuauuagcacaaauaggagaucaauaugcagauuuauuuuuagcagcaaaaaauuuaucagaugcaauauuauuaucagauauauuaagaguaaauacagaaauaacaaaagcaccauuaucagcaucaaugauaaaaagauaugaugaacaucaucaggacuuaacauuauuaaaagcauuaguaagacaacaauuaccagaaaaauauaaagaaauauuuuuugaucaaucaaaaaauggauaugcaggauauauagauggaggagcaucacaagaagaauuuuauaaauuuauaaaaccaauauuagaaaaaauggauggaacagaagaauuauuaguaaaauuaaauagagaagauuuauuaagaaaacaaagaacauuugauaauggaucaauaccacaucaaauacauuuaggagaauuacaugcaauauuaagaagacaagaagauuuuuauccauuuuuaaaagauaauagagaaaaaauagaaaaaauauuaacauuuagaauaccauauuauguaggaccauuagcaagaggaaauucaagauuugcauggaugacaagaaaaucagaagaaacaauaacaccauggaauuuugaagaaguaguagauaaaggagcaucagcacaaucauuuauagaaagaaugacaaauuuugauaaaaauuuaccaaaugaaaaaguauuaccaaaacauucauuauuauaugaauauuuuacaguauauaaugaauuaacaaaaguaaaauauguaacagaaggaaugagaaaaccagcauuuuuaucaggagaacaaaaaaaagcaauaguagauuuauuauuuaaaacaaauagaaaaguaacaguaaaacaauuaaaagaagauuauuuuaaaaaaauagaauguuuugauucaguagaaauaucaggaguagaagauagauuuaaugcaucauuaggaacauaucaugauuuauuaaaaauaauaaaagauaaagauuuuuuagauaaugaagaaaaugaagauauauuagaagauauaguauuaacauuaacauuauuugaagauagagaaaugauagaagaaagauuaaaaacauaugcacauuuauuugaugauaaaguaaugaaacaauuaaaaagaagaagauauacaggauggggaagauuaucaagaaaauuaauaaauggaauaagagauaaacaaucaggaaaaacaauauuagauuuuuuaaaaucagauggauuugcaaauagaaauuuuaugcaauuaauacaugaugauucauuaacauuuaaagaagauauacaaaaagcacaaguaucaggacaaggagauucauuacaugaacauauagcaaauuuagcaggaucaccagcaauaaaaaaaggaauauuacaaacaguaaaaguaguagaugaauuaguaaaaguaaugggaagacauaaaccagaaaauauaguaauagaaauggcaagagaaaaucaaacaacacaaaaaggacaaaaaaauucaagagaaagaaugaaaagaauagaagaaggaauaaaagaauuaggaucacaaauauuaaaagaacauccaguagaaaauacacaauuacaaaaugaaaaauuauauuuauauuauuuacaaaauggaagagauauguauguagaucaagaauuagauauaaauagauuaucagauuaugauguagaucauauaguaccacaaucauuuuuaaaagaugauucaauagauaauaaaguauuaacaagaucagauaaaaauagaggaaaaucagauaauguaccaucagaagaaguaguaaaaaaaaugaaaaauuauuggagacaauuauuaaaugcaaaauuaauaacacaaagaaaauuugauaauuuaacaaaagcagaaagaggaggauuaucagaauuagauaaagcaggauuuauaaaaagacaauuaguagaaacaagacaaauaacaaaacauguagcacaaauauuagauucaagaaugaauacaaaauaugaugaaaaugauaaauuaauaagagaaguaaaaguaauaacauuaaaaucaaaauuaguaucagauuuuagaaaagauuuucaauuuuauaaaguaagagaaauaaauaauuaucaucaugcacaugaugcauauuuaaaugcaguaguaggaacagcauuaauaaaaaaauauccaaaauuagaaucagaauuuguauauggagauuauaaaguauaugauguaagaaaaaugauagcaaaaucagaacaagaaauaggaaaagcaacagcaaaauauuuuuuuuauucaaauauaaugaauuuuuuuaaaacagaaauaacauuagcaaauggagaaauaagaaaaagaccauuaauagaaacaaauggagaaacaggagaaauaguaugggauaaaggaagagauuuugcaacaguaagaaaaguauuaucaaugccacaaguaaauauaguaaaaaaaacagaaguacaaacaggaggauuuucaaaagaaucaauauuaccaaaaagaaauucagauaaauuaauagcaagaaaaaaagauugggauccaaaaaaauauggaggauuugauucaccaacaguagcauauucaguauuaguaguagcaaaaguagaaaaaggaaaaucaaaaaaauuaaaaucaguaaaagaauuauuaggaauaacaauaauggaaagaucaucauuugaaaaaaauccaauagauuuuuuagaagcaaaaggauauaaagaaguaaaaaaagauuuaauaauaaaauuaccaaaauauucauuauuugaauuagaaaauggaagaaaaagaauguuagcaucagcaggagaauuacaaaaaggaaaugaauuagcauuaccaucaaaauauguaaauuuuuuauauuuagcaucacauuaugaaaaauuaaaaggaucaccagaagauaaugaacaaaaacaauuauuuguagaacaacauaaacauuauuuagaugaaauaauagaacaaauaucagaauuuucaaaaagaguaauauuagcagaugcaaauuuagauaaaguauuaucagcauauaauaaacauagagauaaaccaauaagagaacaagcagaaaauauaauacauuuauuuacauuaacaaauuuaggagcaccagcagcauuuaaauauuuugauacaacaauagauagaaaaagauauacaucaacaaaagaaguauuagaugcaacauuaauacaucaaucaauaacaggauuauaugaaacaagaauagauuuaucacaauuaggaggagauggaggaggaucaccaaaaaaaaaaagaaaaguauag

SEQ ID NO: 14 - RNA, 4140 nt, Artificial Sequence
  Synthetic: I-pair enriched Cas9 ORF
auggauaaaaaguacagcaucggauuagauauaggaacaaauucaguuggcugggcugugauaacagaugaauauaaaguucccucaaaaaaauuuaaaguauuaggaaauacagauagacauagcaucaaaaaaaaucucauaggugcacuguuauuugauucaggugagacagcagaagccacaagauuaaaaagaacagcccgcagaagauauacaagaagaaaaaauagaauauguuauuuacaggagauauuuucaaaugaaauggcaaaaguagaugauucauuuuuucauagauuagaagaaucauuccugguagaagaagauaaaaaacaugaaagacauccaauauuuggaaauauaguagaugaagucgcauaucaugaaaaguaccccaccauauaucaucugcggaaaaaauuaguagauucgacugauaaagcagaucugcgguuaauauauuuagcacuggcacauaugauaaaauuuagaggacauuuccugauagaaggagauuuaaauccugacaauucagauguagauaaauuauuuauacaauuaguacaaaccuacaaucaauuauuugaagaaaauccaauaaaugcaucaggaguagaugcaaaagcaauacucagcgcccgccucagcaaaucaagaagauuagaaaaucucauagcacaacuuccaggugagaaaaaaaauggguuauuuggaaaucucauagcacucagcuuaggauuaacucccaauuuuaaaucaaauuuugauuuagcagaagaugcaaaauuacaacucagcaaagauaccuacgaugaugauuuagauaaucucuuagcacaaauaggagaucaauaugcagauuuauuccuggcugccaaaaaucucagcgaugcaauauuacucagcgauauacugcggguaaauacagagauaacaaaagcaccacucagcgcaucaaugauaaaaagauaugaugaacaucaucaagauuuaacauuauuaaaagcacugguaagacaacaacuuccagagaaguacaaagaaauauuuuuugaucagagcaaaaauggguaugccggguauauagaugguggugccucacaggaggaauuuuauaaauuuauaaaaccaauauuagaaaaaauggauggaacagaggagcuguuaguaaaauuaaauagggaggauuuacugcggaaacaaagaacauuugauaaugggagcaucccccaucaaauacauuuaggugagcugcaugcaauacugcggagacaggaggauuuuuauccauuccugaaagauaauagggagaaaauagaaaaaauauuaacauuuagaauccccuauuauguuggcccauuagcccgcggaaauucaagauuugcauggaugacaagaaaaucagaagaaacaauaacucccuggaauuuugaagaagucguagauaagggugccucagcacagagcuuuauagaaagaaugacaaauuuugauaaaaaucuuccaaaugaaaaaguacuuccaaaacauucauuauuauaugaauauuuuacaguauauaaugagcugacaaaaguaaaguacguaacagagggaaugagaaaaccagcauuccucagcggugagcaaaaaaaagcaauaguagauuuauuauuuaaaacaaauagaaaaguaacaguaaaacaauuaaaagaagauuauuuuaaaaaaauagaauguuuugauucaguagaaauaucaggaguagaagauagauuuaaugcaucauuaggaaccuaccaugauuuauuaaaaauaauaaaagauaaagauuuccuggauaaugaagaaaaugaagauauauuagaagauauaguauuaacauuaacauuauuugaagauagggagaugauagaagaaagauuaaaaaccuacgcacauuuauuugaugauaaaguaaugaaacaauuaaaaagaagaagauauacaggauggggaagacucagcagaaaauuaauaaaugggauacgagacaaacagagcggaaaaacaauauuagauuuccugaaaucagauggauuugcaaauagaaauuuuaugcaauuaauacaugaugauucauuaacauuuaaagaagauauacaaaaagcacaggucagcggacagggcgauucauuacaugaacauauagcaaaucucgccgggucaccagcaauaaaaaaggggauauuacaaacaguaaaaguaguagaugagcugguaaaaguaaugggaagacauaaaccagagaauauaguaauagaaauggccagggagaaucaaacaacucaaaaggggcaaaaaaauucaagggagagaaugaaaagaauagaagaaggaauaaaagagcugggaucacaaauauuaaaagaacauccaguagaaaauacucaauuacaaaaugaaaaauuauauuuauauuauuuacaaaaugggcgagacauguauguagaucaggagcuggauauaaauagacucagcgauuaugauguagaucauauaguuccccagagcuuccugaaagaugauagcaucgauaauaaaguauuaacaagaucagauaaaaauagaggaaaaucagauaauguucccucagaagaagucguaaaaaaaaugaaaaauuauuggagacaauuauuaaaugcaaaauuaauaacucaaagaaaauuugauaaucucacaaaagcagaaagagguggccucagcgagcuggauaaagccggguuuauaaaaagacaauuaguagaaacaagacaaauaacaaaacauguagcacaaauauuagauucaagaaugaauacaaaguacgaugaaaaugauaaauuaauaagggaagucaaaguaauaacauuaaaaucaaaauuagucagcgauuuuagaaaagauuuucaauuuuauaaaguaagggagauaaauaauuaucaucaugcacaugaugcauauuuaaaugcugugguuggcacagcacugauaaaaaaguacccaaaauuagaaucagaauuuguauauggagauuauaaaguauaugauguaagaaaaaugauagcaaaaucagaacaggagauaggaaaagcaacagcaaaguacuuuuuuuauucaaauauaaugaauuuuuuuaaaacagagauaacauuagcaaauggugagauaagaaaaagaccauuaauagaaacaaauggugagacaggugagauaguaugggauaaggggcgagacuuugcaacaguaagaaaaguacucagcaugccacaggugaauauaguaaaaaaaacagaaguccaaacagguggcuuuucaaaagaaagcauccuuccaaaaagaaauucagauaaauuaauagcccgcaaaaaagauugggauccaaaaaaguacgguggcuuugauucacccaccguagcauauucaguauuaguaguagcaaaaguagaaaaggggaaaucaaaaaaauuaaaaucaguaaaagagcuguuaggaauaacaauaauggaaagaucaucauuugaaaaaaauccaauagauuuccuggaagccaagggguauaaagaagucaaaaaagauuuaauaauaaaacuuccaaaguacucauuauuugagcuggaaaaugggagaaaaagaauguuagcaucagccggugagcugcaaaaggggaaugagcuggcacuucccucaaaguacguaaauuuccuguauuuagcaucacauuaugaaaaauuaaaggggucaccagaggauaaugaacaaaaacaauuauuuguagaacaacauaaacauuauuuagaugaaauaauagaacaaauaucagaauuuucaaaaagaguaauauuagcagaugcaaaucucgauaaaguacucagcgcauauaauaaacaucgagacaaaccaauaagggagcaggccgaaaauauaauacauuuauuuacauuaacaaaucucggugccccagcugccuuuaaguacuuugauacaacaauagauagaaaaagauauacaucgacuaaagaagucuuagaugcaacauuaauacaucagagcaucacaggauuauaugaaacaagaauagaucucagccaauuagguggcgauggugguggcucaccaaaaaaaaaaagaaaaguauag

SEQ ID NO: 15 - RNA, 4425 nt, Artificial Sequence
  Synthetic: Cas9 mRNA transcript comprising SEQ 5
gggaagcucagaauaaacgcucaacuuuggccggaucugccaccauggacaagaaguacagcaucggccuggacaucggcacgaacagcgugggcugggccgugaucacggacgaguacaaggugcccagcaagaaguucaaggugcugggcaacacggaccggcacagcaucaagaagaaccugaucggcgcccugcuguucgacagcggcgagacggccgaggccacgcggcugaagcggacggcccggcggcgguacacgcggcggaagaaccggaucugcuaccugcaggagaucuucagcaacgagauggccaagguggacgacagcuucuuccaccggcuggaggagagcuuccugguggaggaggacaagaagcacgagcggcaccccaucuucggcaacaucguggacgagguggccuaccacgagaaguaccccacgaucuaccaccugcggaagaagcugguggacagcacggacaaggccgaccugcggcugaucuaccuggcccuggcccacaugaucaaguuccggggccacuuccugaucgagggcgaccugaaccccgacaacagcgacguggacaagcuguucauccagcuggugcagacguacaaccagcuguucgaggagaaccccaucaacgccagcggcguggacgccaaggccauccugagcgcccggcugagcaagagccggcggcuggagaaccugaucgcccagcugcccggcgagaagaagaacggccuguucggcaaccugaucgcccugagccugggccugacgcccaacuucaagagcaacuucgaccuggccgaggacgccaagcugcagcugagcaaggacacguacgacgacgaccuggacaaccugcuggcccagaucggcgaccaguacgccgaccuguuccuggccgccaagaaccugagcgacgccauccugcugagcgacauccugcgggugaacacggagaucacgaaggccccccugagcgccagcaugaucaagcgguacgacgagcaccaccaggaccugacgcugcugaaggcccuggugcggcagcagcugcccgagaaguacaaggagaucuucuucgaccagagcaagaacggcuacgccggcuacaucgacggcggcgccagccaggaggaguucuacaaguucaucaagcccauccuggagaagauggacggcacggaggagcugcuggugaagcugaaccgggaggaccugcugcggaagcagcggacguucgacaacggcagcaucccccaccagauccaccugggcgagcugcacgccauccugcggcggcaggaggacuucuaccccuuccugaaggacaaccgggagaagaucgagaagauccugacguuccggauccccuacuacgugggcccccuggcccggggcaacagccgguucgccuggaugacgcggaagagcgaggagacgaucacgcccuggaacuucgaggaggugguggacaagggcgccagcgcccagagcuucaucgagcggaugacgaacuucgacaagaaccugcccaacgagaaggugcugcccaagcacagccugcuguacgaguacuucacgguguacaacgagcugacgaaggugaaguacgugacggagggcaugcggaagcccgccuuccugagcggcgagcagaagaaggccaucguggaccugcuguucaagacgaaccggaaggugacggugaagcagcugaaggaggacuacuucaagaagaucgagugcuucgacagcguggagaucagcggcguggaggaccgguucaacgccagccugggcacguaccacgaccugcugaagaucaucaaggacaaggacuuccuggacaacgaggagaacgaggacauccuggaggacaucgugcugacgcugacgcuguucgaggaccgggagaugaucgaggagcggcugaagacguacgcccaccuguucgacgacaaggugaugaagcagcugaagcggcggcgguacacgggcuggggccggcugagccggaagcugaucaacggcauccgggacaagcagagcggcaagacgauccuggacuuccugaagagcgacggcuucgccaaccggaacuucaugcagcugauccacgacgacagccugacguucaaggaggacauccagaaggcccaggugagcggccagggcgacagccugcacgagcacaucgccaaccuggccggcagccccgccaucaagaagggcauccugcagacggugaaggugguggacgagcuggugaaggugaugggccggcacaagcccgagaacaucgugaucgagauggcccgggagaaccagacgacgcagaagggccagaagaacagccgggagcggaugaagcggaucgaggagggcaucaaggagcugggcagccagauccugaaggagcaccccguggagaacacgcagcugcagaacgagaagcuguaccuguacuaccugcagaacggccgggacauguacguggaccaggagcuggacaucaaccggcugagcgacuacgacguggaccacaucgugccccagagcuuccugaaggacgacagcaucgacaacaaggugcugacgcggagcgacaagaaccggggcaagagcgacaacgugcccagcgaggagguggugaagaagaugaagaacuacuggcggcagcugcugaacgccaagcugaucacgcagcggaaguucgacaaccugacgaaggccgagcggggcggccugagcgagcuggacaaggccggcuucaucaagcggcagcugguggagacgcggcagaucacgaagcacguggcccagauccuggacagccggaugaacacgaaguacgacgagaacgacaagcugauccgggaggugaaggugaucacgcugaagagcaagcuggugagcgacuuccggaaggacuuccaguucuacaaggugcgggagaucaacaacuaccaccacgcccacgacgccuaccugaacgccguggugggcacggcccugaucaagaaguaccccaagcuggagagcgaguucguguacggcgacuacaagguguacgacgugcggaagaugaucgccaagagcgagcaggagaucggcaaggccacggccaaguacuucuucuacagcaacaucaugaacuucuucaagacggagaucacgcuggccaacggcgagauccggaagcggccccugaucgagacgaacggcgagacgggcgagaucgugugggacaagggccgggacuucgccacggugcggaaggugcugagcaugccccaggugaacaucgugaagaagacggaggugcagacgggcggcuucagcaaggagagcauccugcccaagcggaacagcgacaagcugaucgcccggaagaaggacugggaccccaagaaguacggcggcuucgacagccccacgguggccuacagcgugcuggugguggccaagguggagaagggcaagagcaagaagcugaagagcgugaaggagcugcugggcaucacgaucauggagcggagcagcuucgagaagaaccccaucgacuuccuggaggccaagggcuacaaggaggugaagaaggaccugaucaucaagcugcccaaguacagccuguucgagcuggagaacggccggaagcggaugcuggccagcgccggcgagcugcagaagggcaacgagcuggcccugcccagcaaguacgugaacuuccuguaccuggccagccacuacgagaagcugaagggcagccccgaggacaacgagcagaagcagcuguucguggagcagcacaagcacuaccuggacgagaucaucgagcagaucagcgaguucagcaagcgggugauccuggccgacgccaaccuggacaaggugcugagcgccuacaacaagcaccgggacaagcccauccgggagcaggccgagaacaucauccaccuguucacgcugacgaaccugggcgcccccgccgccuucaaguacuucgacacgacgaucgaccggaagcgguacacgagcacgaaggaggugcuggacgccacgcugauccaccagagcaucacgggccuguacgagacgcggaucgaccugagccagcugggcggcgacggcggcggcagccccaagaagaagcggaagguguagcuagcaccagccucaagaacacccgaauggagucucuaagcuacauaauaccaacuuacacuuuacaaaauguugucccccaaaauguagccauucguaucugcuccuaauaaaaagaaaguuucuucacauucucucgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaucuag

SEQ ID NO: 16 - RNA, 4425 nt, Artificial Sequence
  Synthetic: Cas9 mRNA transcript comprising SEQ 7
gggaagcucagaauaaacgcucaacuuuggccggaucugccaccauggacaagaaguacagcaucggccucgacaucggcaccaacagcguuggcugggcugugaucaccgacgaguacaagguucccucaaagaaguucaagguccucggcaacaccgaccgccacagcaucaagaagaaucucaucggugcacugcucuucgacagcggugagaccgcggaagccacccgccucaagcggaccgcccgccgccgcuacacccgccgcaagaaccgcaucugcuaccuccaggagaucuucagcaacgagauggccaaggucgacgacagcuucuuccaccgccucgaggagagcuuccuggucgaggaggacaagaagcacgagcgccaccccaucuucggcaacaucgucgacgaagucgccuaccacgagaaguaccccaccaucuaccaccugcggaagaagcucgucgacucgacugacaaggccgaccugcggcucaucuaccucgcacuggcccacaugauaaaguuccgcggccacuuccugaucgagggcgaccucaacccugacaacagcgacgucgacaagcucuucauccagcucguccagaccuacaaccagcucuucgaggagaaccccaucaacgccagcggcgucgacgccaaggccauccucagcgcccgccucagcaagagccgccgccucgagaaucucaucgcccagcuuccaggugagaagaagaaugggcucuucggcaaucucaucgcacucagccucggccucacucccaacuucaagagcaacuucgaccucgcggaggacgccaagcuccagcucagcaaggacaccuacgacgacgaccucgacaaucuccucgcccagaucggcgaccaguacgccgaccucuuccuggcugccaagaaucucagcgacgccauccuccucagcgacauccugcgggucaacacagagaucaccaaggccccccucagcgccagcaugauaaagcgcuacgacgagcaccaccaggaccucacccuccucaaggcacugguccgccagcagcuuccagagaaguacaaggagaucuucuucgaccagagcaagaauggguacgccggguacaucgacgguggugccagccaggaggaguucuacaaguucaucaagcccauccucgagaagauggacggcacagaggagcugcucgucaagcucaacagggaggaccuccugcggaagcagcggaccuucgacaaugggagcaucccccaccagauccaccucggugagcugcacgccauccugcggcgccaggaggacuucuaccccuuccugaaggacaacagggagaagaucgagaagauccucaccuuccgcauccccuacuacguuggcccccucgcccgcggcaacagccgcuucgccuggaugacccgcaagagcgaggagaccaucacucccuggaacuucgaggaagucgucgacaagggugccagcgcccagagcuucaucgagcgcaugaccaacuucgacaagaaucuuccaaacgagaagguccuuccaaagcacagccuccucuacgaguacuucaccgucuacaacgagcugaccaaggucaaguacgucacagagggcaugcgcaagccagccuuccucagcggugagcagaagaaggccaucgucgaccuccucuucaagaccaaccgcaaggucaccgucaagcagcucaaggaggacuacuucaagaagaucgagugcuucgacagcgucgagaucagcggcgucgaggaccgcuucaacgccagccucggcaccuaccacgaccuccucaagaucaucaaggacaaggacuuccuggacaacgaggagaacgaggacauccucgaggacaucguccucacccucacccucuucgaggacagggagaugauagaggagcgccucaagaccuacgcccaccucuucgacgacaaggucaugaagcagcucaagcgccgccgcuacaccggcuggggccgccucagccgcaagcucaucaaugggauccgagacaagcagagcggcaagaccauccucgacuuccugaagagcgacggcuucgccaaccgcaacuucaugcagcucauccacgacgacagccucaccuucaaggaggacauccagaaggcccaggucagcggccagggcgacagccuccacgagcacaucgccaaucucgccgggagcccagccaucaagaaggggauccuccagaccgucaaggucgucgacgagcuggucaaggucaugggccgccacaagccagagaacaucgucaucgagauggccagggagaaccagaccacucaaaaggggcagaagaacagcagggagcgcaugaagcgcaucgaggagggcaucaaggagcugggcagccagauccucaaggagcaccccgucgagaacacucaacuccagaacgagaagcucuaccucuacuaccuccagaaugggcgagacauguacgucgaccaggagcuggacaucaaccgccucagcgacuacgacgucgaccacaucguuccccagagcuuccugaaggacgacagcaucgacaacaagguccucacccgaagcgacaagaaccgcggcaagagcgacaacguucccucagaggaagucgucaagaagaugaagaacuacuggcgccagcuccucaacgccaagcucaucacucaacgcaaguucgacaaucucaccaaggcggagcgcgguggccucagcgagcuggacaaggccggguucaucaagcgccagcucgucgagacccgccagaucaccaagcacgucgcccagauccucgacagccgcaugaacaccaaguacgacgagaacgacaagcucaucagggaagucaaggucaucacccucaagagcaagcucgucagcgacuuccgcaaggacuuccaguucuacaaggucagggagaucaacaacuaccaccacgcccacgacgccuaccucaacgcugugguuggcaccgcacugaucaagaaguaccccaagcucgagagcgaguucgucuacggcgacuacaaggucuacgacguccgcaagaugauagccaagagcgagcaggagaucggcaaggccaccgccaaguacuucuucuacagcaacaucaugaacuucuucaagacagagaucacccucgccaauggugagauccgcaagcgcccccucaucgagaccaauggugagaccggugagaucgucugggacaaggggcgagacuucgccaccguccgcaagguccucagcaugccccaggugaacaucgucaagaagacagaaguccagaccgguggcuucagcaaggagagcauccuuccaaagcgcaacagcgacaagcucaucgcccgcaagaaggacugggaccccaagaaguacgguggcuucgacagccccaccgucgccuacagcguccucgucgucgccaaggucgagaaggggaagagcaagaagcucaagagcgucaaggagcugcucggcaucaccaucauggagcgaagcagcuucgagaagaaccccaucgacuuccuggaagccaagggguacaaggaagucaagaaggaccucaucaucaagcuuccaaaguacagccucuucgagcuggagaaugggcgcaagcgcaugcucgccagcgccggugagcugcagaaggggaacgagcuggcacuucccucaaaguacgucaacuuccuguaccucgccagccacuacgagaagcucaaggggagcccagaggacaacgagcagaagcagcucuucgucgagcagcacaagcacuaccucgacgagaucaucgagcagaucagcgaguucagcaagcgcgucauccucgccgacgccaaucucgacaagguccucagcgccuacaacaagcaccgagacaagcccaucagggagcaggccgagaacaucauccaccucuucacccucaccaaucucggugccccagcugccuucaaguacuucgacaccaccaucgaccgcaagcgcuacaccucgacuaaggaaguccucgacgccacccucauccaccagagcaucaccggccucuacgagacccgcaucgaccucagccagcucgguggcgacggugguggcagccccaagaagaagcgcaaggucuagcuagcaccagccucaagaacacccgaauggagucucuaagcuacauaauaccaacuuacacuuuacaaaauguugucccccaaaauguagccauucguaucugcuccuaauaaaaagaaaguuucuucacauucucucgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaucuag

SEQ ID NO: 17 - RNA, 4425 nt, Artificial Sequence
  Synthetic: Cas9 mRNA transcript comprising SEQ 9
gggaagcucagaauaaacgcucaacuuuggccggaucugccaccauggacaagaaguacagcaucggccucgacaucggcaccaacagcguuggcugggcugugaucaccgacgaguacaagguucccucaaagaaguucaagguccucggcaacaccgaccgccacagcaucaagaagaaucucaucggugcacugcucuucgacagcggugagaccgcggaagccacccgccucaagcgcaccgcccgccgccgcuacacccgccgcaagaaccgcaucugcuaccuccaggagaucuucagcaacgagauggccaaggucgacgacagcuucuuccaccgccucgaggagagcuuccuggucgaggaggacaagaagcacgagcgccaccccaucuucggcaacaucgucgacgaagucgccuaccacgagaaguaccccaccaucuaccaccugcggaagaagcucgucgacucgacugacaaggccgaccugcggcucaucuaccucgcacuggcccacaugauaaaguuccgcggccacuuccugaucgagggcgaccucaacccugacaacagcgacgucgacaagcucuucauccagcucguccagaccuacaaccagcucuucgaggagaaccccaucaacgccagcggcgucgacgccaaggccauccucagcgcccgccucagcaagagccgccgccucgagaaucucaucgcccagcuuccaggugagaagaagaaugggcucuucggcaaucucaucgcacucagccucggccucacucccaacuucaagagcaacuucgaccucgcggaggacgccaagcuccagcucagcaaggacaccuacgacgacgaccucgacaaucuccucgcccagaucggcgaccaguacgccgaccucuuccuggcugccaagaaucucagcgacgccauccuccucagcgacauccugcgggucaacacagagaucaccaaggccccccucagcgccagcaugauaaagcgcuacgacgagcaccaccaggaccucacccuccucaaggcacugguccgccagcagcuuccagagaaguacaaggagaucuucuucgaccagagcaagaauggguacgccggguacaucgacgguggugccagccaggaggaguucuacaaguucaucaagcccauccucgagaagauggacggcacagaggagcugcucgucaagcucaacagggaggaccuccugcggaagcagcgcaccuucgacaaugggagcaucccccaccagauccaccucggugagcugcacgccauccugcggcgccaggaggacuucuaccccuuccugaaggacaacagggagaagaucgagaagauccucaccuuccgcauccccuacuacguuggcccccucgcccgcggcaacagccgcuucgccuggaugacccgcaagagcgaggagaccaucacucccuggaacuucgaggaagucgucgacaagggugccagcgcccagagcuucaucgagcgcaugaccaacuucgacaagaaucuuccaaacgagaagguccuuccaaagcacagccuccucuacgaguacuucaccgucuacaacgagcugaccaaggucaaguacgucacagagggcaugcgcaagccagccuuccucagcggugagcagaagaaggccaucgucgaccuccucuucaagaccaaccgcaaggucaccgucaagcagcucaaggaggacuacuucaagaagaucgagugcuucgacagcgucgagaucagcggcgucgaggaccgcuucaacgccagccucggcaccuaccacgaccuccucaagaucaucaaggacaaggacuuccuggacaacgaggagaacgaggacauccucgaggacaucguccucacccucacccucuucgaggacagggagaugauagaggagcgccucaagaccuacgcccaccucuucgacgacaaggucaugaagcagcucaagcgccgccgcuacaccggcuggggccgccucagccgcaagcucaucaaugggauccgagacaagcagagcggcaagaccauccucgacuuccugaagagcgacggcuucgccaaccgcaacuucaugcagcucauccacgacgacagccucaccuucaaggaggacauccagaaggcccaggucagcggccagggcgacagccuccacgagcacaucgccaaucucgccgggagcccagccaucaagaaggggauccuccagaccgucaaggucgucgacgagcuggucaaggucaugggccgccacaagccagagaacaucgucaucgagauggccagggagaaccagaccacucaaaaggggcagaagaacagcagggagcgcaugaagcgcaucgaggagggcaucaaggagcugggcagccagauccucaaggagcaccccgucgagaacacucaacuccagaacgagaagcucuaccucuacuaccuccagaaugggcgagacauguacgucgaccaggagcuggacaucaaccgccucagcgacuacgacgucgaccacaucguuccccagagcuuccugaaggacgacagcaucgacaacaagguccucacccgaagcgacaagaaccgcggcaagagcgacaacguucccucagaggaagucgucaagaagaugaagaacuacuggcgccagcuccucaacgccaagcucaucacucaacgcaaguucgacaaucucaccaaggcggagcgcgguggccucagcgagcuggacaaggccggguucaucaagcgccagcucgucgagacccgccagaucaccaagcacgucgcccagauccucgacagccgcaugaacaccaaguacgacgagaacgacaagcucaucagggaagucaaggucaucacccucaagagcaagcucgucagcgacuuccgcaaggacuuccaguucuacaaggucagggagaucaacaacuaccaccacgcccacgacgccuaccucaacgcugugguuggcaccgcacugaucaagaaguaccccaagcucgagagcgaguucgucuacggcgacuacaaggucuacgacguccgcaagaugauagccaagagcgagcaggagaucggcaaggccaccgccaaguacuucuucuacagcaacaucaugaacuucuucaagacagagaucacccucgccaauggugagauccgcaagcgcccccucaucgagaccaauggugagaccggugagaucgucugggacaaggggcgagacuucgccaccguccgcaagguccucagcaugccccaggugaacaucgucaagaagacagaaguccagaccgguggcuucagcaaggagagcauccuuccaaagcgcaacagcgacaagcucaucgcccgcaagaaggacugggaccccaagaaguacgguggcuucgacagccccaccgucgccuacagcguccucgucgucgccaaggucgagaaggggaagagcaagaagcucaagagcgucaaggagcugcucggcaucaccaucauggagcgaagcagcuucgagaagaaccccaucgacuuccuggaagccaagggguacaaggaagucaagaaggaccucaucaucaagcuuccaaaguacagccucuucgagcuggagaaugggcgcaagcgcaugcucgccagcgccggugagcugcagaaggggaacgagcuggcacuucccucaaaguacgucaacuuccuguaccucgccagccacuacgagaagcucaaggggagcccagaggacaacgagcagaagcagcucuucgucgagcagcacaagcacuaccucgacgagaucaucgagcagaucagcgaguucagcaagcgcgucauccucgccgacgccaaucucgacaagguccucagcgccuacaacaagcaccgagacaagcccaucagggagcaggccgagaacaucauccaccucuucacccucaccaaucucggugccccagcugccuucaaguacuucgacaccaccaucgaccgcaagcgcuacaccucgacuaaggaaguccucgacgccacccucauccaccagagcaucaccggccucuacgagacccgcaucgaccucagccagcucgguggcgacggugguggcagccccaagaagaagcgcaaggucuagcuagcaccagccucaagaacacccgaauggagucucuaagcuacauaauaccaacuuacacuuuacaaaauguugucccccaaaauguagccauucguaucugcuccuaauaaaaagaaaguuucuucacauucucucgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaucuag

SEQ ID NO: 18 - RNA, 4425 nt, Artificial Sequence
  Synthetic: Cas9 transcript comprising SEQ 8
gggaagcucagaauaaacgcucaacuuuggccggaucugccaccauggacaagaaguacagcaucggccuggacaucggcacgaacagcguuggcugggcugugaucacggacgaguacaagguucccucaaagaaguucaaggugcugggcaacacggaccggcacagcaucaagaagaaucucaucggugcacugcuguucgacagcggugagacggccgaagccacgcggcugaagcggacggcccgccggcgguacacgcggcggaagaaccggaucugcuaccugcaggagaucuucagcaacgagauggccaagguggacgacagcuucuuccaccggcuggaggagagcuuccugguggaggaggacaagaagcacgagcggcaccccaucuucggcaacaucguggacgaagucgccuaccacgagaaguaccccaccaucuaccaccugcggaagaagcugguggacucgacugacaaggccgaccugcggcugaucuaccuggcacuggcccacaugauaaaguuccggggccacuuccugaucgagggcgaccugaacccugacaacagcgacguggacaagcuguucauccagcuggugcagaccuacaaccagcuguucgaggagaaccccaucaacgccagcggcguggacgccaaggccauccucagcgcccgccucagcaagagccggcggcuggagaaucucaucgcccagcuuccaggugagaagaagaaugggcuguucggcaaucucaucgcacucagccugggccugacucccaacuucaagagcaacuucgaccuggccgaggacgccaagcugcagcucagcaaggacaccuacgacgacgaccuggacaaucuccuggcccagaucggcgaccaguacgccgaccuguuccuggcugccaagaaucucagcgacgccauccugcucagcgacauccugcgggugaacacagagaucacgaaggccccccucagcgccagcaugauaaagcgguacgacgagcaccaccaggaccugacgcugcugaaggcacuggugcggcagcagcuuccagagaaguacaaggagaucuucuucgaccagagcaagaauggguacgccggguacaucgacgguggugccagccaggaggaguucuacaaguucaucaagcccauccuggagaagauggacggcacagaggagcugcuggugaagcugaacagggaggaccugcugcggaagcagcggacguucgacaaugggagcaucccccaccagauccaccugggugagcugcacgccauccugcggcggcaggaggacuucuaccccuuccugaaggacaacagggagaagaucgagaagauccugacguuccggauccccuacuacguuggcccccuggcccgcggcaacagccgguucgccuggaugacgcggaagagcgaggagacgaucacucccuggaacuucgaggaagucguggacaagggugccagcgcccagagcuucaucgagcggaugacgaacuucgacaagaaucuuccaaacgagaaggugcuuccaaagcacagccugcuguacgaguacuucacgguguacaacgagcugacgaaggugaaguacgugacagagggcaugcggaagcccgccuuccucagcggugagcagaagaaggccaucguggaccugcuguucaagacgaaccggaaggugacggugaagcagcugaaggaggacuacuucaagaagaucgagugcuucgacagcguggagaucagcggcguggaggaccgguucaacgccagccugggcaccuaccacgaccugcugaagaucaucaaggacaaggacuuccuggacaacgaggagaacgaggacauccuggaggacaucgugcugacgcugacgcuguucgaggacagggagaugauagaggagcggcugaagaccuacgcccaccuguucgacgacaaggugaugaagcagcugaagcggcggcgguacacgggcuggggccggcucagccggaagcugaucaaugggauccgagacaagcagagcggcaagacgauccuggacuuccugaagagcgacggcuucgccaaccggaacuucaugcagcugauccacgacgacagccugacguucaaggaggacauccagaaggcccaggucagcggccagggcgacagccugcacgagcacaucgccaaucucgccgggagccccgccaucaagaaggggauccugcagacggugaaggugguggacgagcuggugaaggugaugggccggcacaagccagagaacaucgugaucgagauggccagggagaaccagacgacucaaaaggggcagaagaacagcagggagcggaugaagcggaucgaggagggcaucaaggagcugggcagccagauccugaaggagcaccccguggagaacacucaacugcagaacgagaagcuguaccuguacuaccugcagaaugggcgagacauguacguggaccaggagcuggacaucaaccggcucagcgacuacgacguggaccacaucguuccccagagcuuccugaaggacgacagcaucgacaacaaggugcugacgcggagcgacaagaaccggggcaagagcgacaacguucccucagaggaagucgugaagaagaugaagaacuacuggcggcagcugcugaacgccaagcugaucacucaacggaaguucgacaaucucacgaaggccgagcgggguggccucagcgagcuggacaaggccggguucaucaagcggcagcugguggagacgcggcagaucacgaagcacguggcccagauccuggacagccggaugaacacgaaguacgacgagaacgacaagcugaucagggaagucaaggugaucacgcugaagagcaagcuggucagcgacuuccggaaggacuuccaguucuacaaggugagggagaucaacaacuaccaccacgcccacgacgccuaccugaacgcugugguuggcacggcacugaucaagaaguaccccaagcuggagagcgaguucguguacggcgacuacaagguguacgacgugcggaagaugauagccaagagcgagcaggagaucggcaaggccacggccaaguacuucuucuacagcaacaucaugaacuucuucaagacagagaucacgcuggccaauggugagauccggaagcggccccugaucgagacgaauggugagacgggugagaucgugugggacaaggggcgagacuucgccacggugcggaaggugcucagcaugccccaggugaacaucgugaagaagacagaaguccagacggguggcuucagcaaggagagcauccuuccaaagcggaacagcgacaagcugaucgcccgcaagaaggacugggaccccaagaaguacgguggcuucgacagccccaccguggccuacagcgugcuggugguggccaagguggagaaggggaagagcaagaagcugaagagcgugaaggagcugcugggcaucacgaucauggagcggagcagcuucgagaagaaccccaucgacuuccuggaagccaagggguacaaggaagucaagaaggaccugaucaucaagcuuccaaaguacagccuguucgagcuggagaaugggcggaagcggaugcuggccagcgccggugagcugcagaaggggaacgagcuggcacuucccucaaaguacgugaacuuccuguaccuggccagccacuacgagaagcugaaggggagcccagaggacaacgagcagaagcagcuguucguggagcagcacaagcacuaccuggacgagaucaucgagcagaucagcgaguucagcaagcgggugauccuggccgacgccaaucucgacaaggugcucagcgccuacaacaagcaccgagacaagcccaucagggagcaggccgagaacaucauccaccuguucacgcugacgaaucucggugcccccgcugccuucaaguacuucgacacgacgaucgaccggaagcgguacacgucgacuaaggaaguccuggacgccacgcugauccaccagagcaucacgggccuguacgagacgcggaucgaccucagccagcuggguggcgacggugguggcagccccaagaagaagcggaagguguagcuagcaccagccucaagaacacccgaauggagucucuaagcuacauaauaccaacuuacacuuuacaaaauguugucccccaaaauguagccauucguaucugcuccuaauaaaaagaaaguuucuucacauucucucgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaucuag

SEQ ID NO: 19 - RNA, 4434 nt, Artificial Sequence
  Synthetic: Cas9 mRNA transcript comprising SEQ 10
gggaagcucagaauaaacgcucaacuuuggccggaucugccaccauggacaagaaguacagcaucggccucgacaucggcaccaacagcgucggcugggccgucaucaccgacgaguacaagguccccagcaagaaguucaagguccucggcaacaccgaccgccacagcaucaagaagaaccucaucggcgcccuccucuucgacagcggcgagaccgccgaggccacccgccucaagcgcaccgcccgccgccgcuacacccgccgcaagaaccgcaucugcuaccuccaggagaucuucagcaacgagauggccaaggucgacgacagcuucuuccaccgccucgaggagagcuuccucgucgaggaggacaagaagcacgagcgccaccccaucuucggcaacaucgucgacgaggucgccuaccacgagaaguaccccaccaucuaccaccuccgcaagaagcucgucgacagcaccgacaaggccgaccuccgccucaucuaccucgcccucgcccacaugaucaaguuccgcggccacuuccucaucgagggcgaccucaaccccgacaacagcgacgucgacaagcucuucauccagcucguccagaccuacaaccagcucuucgaggagaaccccaucaacgccagcggcgucgacgccaaggccauccucagcgcccgccucagcaagagccgccgccucgagaaccucaucgcccagcuccccggcgagaagaagaacggccucuucggcaaccucaucgcccucagccucggccucacccccaacuucaagagcaacuucgaccucgccgaggacgccaagcuccagcucagcaaggacaccuacgacgacgaccucgacaaccuccucgcccagaucggcgaccaguacgccgaccucuuccucgccgccaagaaccucagcgacgccauccuccucagcgacauccuccgcgucaacaccgagaucaccaaggccccccucagcgccagcaugaucaagcgcuacgacgagcaccaccaggaccucacccuccucaaggcccucguccgccagcagcuccccgagaaguacaaggagaucuucuucgaccagagcaagaacggcuacgccggcuacaucgacggcggcgccagccaggaggaguucuacaaguucaucaagcccauccucgagaagauggacggcaccgaggagcuccucgucaagcucaaccgcgaggaccuccuccgcaagcagcgcaccuucgacaacggcagcaucccccaccagauccaccucggcgagcuccacgccauccuccgccgccaggaggacuucuaccccuuccucaaggacaaccgcgagaagaucgagaagauccucaccuuccgcauccccuacuacgucggcccccucgcccgcggcaacagccgcuucgccuggaugacccgcaagagcgaggagaccaucacccccuggaacuucgaggaggucgucgacaagggcgccagcgcccagagcuucaucgagcgcaugaccaacuucgacaagaaccuccccaacgagaagguccuccccaagcacagccuccucuacgaguacuucaccgucuacaacgagcucaccaaggucaaguacgucaccgagggcaugcgcaagcccgccuuccucagcggcgagcagaagaaggccaucgucgaccuccucuucaagaccaaccgcaaggucaccgucaagcagcucaaggaggacuacuucaagaagaucgagugcuucgacagcgucgagaucagcggcgucgaggaccgcuucaacgccagccucggcaccuaccacgaccuccucaagaucaucaaggacaaggacuuccucgacaacgaggagaacgaggacauccucgaggacaucguccucacccucacccucuucgaggaccgcgagaugaucgaggagcgccucaagaccuacgcccaccucuucgacgacaaggucaugaagcagcucaagcgccgccgcuacaccggcuggggccgccucagccgcaagcucaucaacggcauccgcgacaagcagagcggcaagaccauccucgacuuccucaagagcgacggcuucgccaaccgcaacuucaugcagcucauccacgacgacagccucaccuucaaggaggacauccagaaggcccaggucagcggccagggcgacagccuccacgagcacaucgccaaccucgccggcagccccgccaucaagaagggcauccuccagaccgucaaggucgucgacgagcucgucaaggucaugggccgccacaagcccgagaacaucgucaucgagauggcccgcgagaaccagaccacccagaagggccagaagaacagccgcgagcgcaugaagcgcaucgaggagggcaucaaggagcucggcagccagauccucaaggagcaccccgucgagaacacccagcuccagaacgagaagcucuaccucuacuaccuccagaacggccgcgacauguacgucgaccaggagcucgacaucaaccgccucagcgacuacgacgucgaccacaucgucccccagagcuuccucaaggacgacagcaucgacaacaagguccucacccgcagcgacaagaaccgcggcaagagcgacaacguccccagcgaggaggucgucaagaagaugaagaacuacuggcgccagcuccucaacgccaagcucaucacccagcgcaaguucgacaaccucaccaaggccgagcgcggcggccucagcgagcucgacaaggccggcuucaucaagcgccagcucgucgagacccgccagaucaccaagcacgucgcccagauccucgacagccgcaugaacaccaaguacgacgagaacgacaagcucauccgcgaggucaaggucaucacccucaagagcaagcucgucagcgacuuccgcaaggacuuccaguucuacaagguccgcgagaucaacaacuaccaccacgcccacgacgccuaccucaacgccgucgucggcaccgcccucaucaagaaguaccccaagcucgagagcgaguucgucuacggcgacuacaaggucuacgacguccgcaagaugaucgccaagagcgagcaggagaucggcaaggccaccgccaaguacuucuucuacagcaacaucaugaacuucuucaagaccgagaucacccucgccaacggcgagauccgcaagcgcccccucaucgagaccaacggcgagaccggcgagaucgucugggacaagggccgcgacuucgccaccguccgcaagguccucagcaugccccaggucaacaucgucaagaagaccgagguccagaccggcggcuucagcaaggagagcauccuccccaagcgcaacagcgacaagcucaucgcccgcaagaaggacugggaccccaagaaguacggcggcuucgacagccccaccgucgccuacagcguccucgucgucgccaaggucgagaagggcaagagcaagaagcucaagagcgucaaggagcuccucggcaucaccaucauggagcgcagcagcuucgagaagaaccccaucgacuuccucgaggccaagggcuacaaggaggucaagaaggaccucaucaucaagcuccccaaguacagccucuucgagcucgagaacggccgcaagcgcaugcucgccagcgccggcgagcuccagaagggcaacgagcucgcccuccccagcaaguacgucaacuuccucuaccucgccagccacuacgagaagcucaagggcagccccgaggacaacgagcagaagcagcucuucgucgagcagcacaagcacuaccucgacgagaucaucgagcagaucagcgaguucagcaagcgcgucauccucgccgacgccaaccucgacaagguccucagcgccuacaacaagcaccgcgacaagcccauccgcgagcaggccgagaacaucauccaccucuucacccucaccaaccucggcgcccccgccgccuucaaguacuucgacaccaccaucgaccgcaagcgcuacaccagcaccaaggagguccucgacgccacccucauccaccagagcaucaccggccucuacgagacccgcaucgaccucagccagcucggcggcgacggcggcggcagccccaagaagaagcgcaaggucuagcuagcaccagccucaagaacacccgaauggagucucuaagcuacauaauaccaacuuacacuuuacaaaauguugucccccaaaauguagccauucguaucugcuccuaauaaaaagaaaguuucuucacauucucucgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaucuag

SEQ ID NO: 20 - RNA, 4431 nt, Artificial Sequence
  Synthetic: Cas9 transcript comprising SEQ 6
gggaagcucagaauaaacgcucaacuuuggccggaucugccaccauggacaagaaguacagcaucggccuggacaucggcacgaacagcguuggcugggcugugaucacggacgaguacaagguucccagcaagaaguucaaggugcugggcaacacggaccggcacagcaucaagaagaaucugaucggugcacugcuguucgacagcggugagacggccgaagccacgcggcugaagcggacggcccggcggcgguacacgcggcggaagaaccggaucugcuaccugcaggagaucuucagcaacgagauggccaagguggacgacagcuucuuccaccggcuggaggagagcuuccugguggaggaggacaagaagcacgagcggcaccccaucuucggcaacaucguggacgaaguggccuaccacgagaaguaccccacgaucuaccaccugcggaagaagcugguggacucgacggacaaggccgaccugcggcugaucuaccuggcacuggcccacaugaucaaguuccggggccacuuccugaucgagggcgaccugaacccugacaacagcgacguggacaagcuguucauccagcuggugcagaccuacaaccagcuguucgaggagaaccccaucaacgccagcggcguggacgccaaggccauccucagcgcccggcucagcaagagccggcggcuggagaaucugaucgcccagcuucccggugagaagaagaauggccuguucggcaaucugaucgcacucagccugggccugacucccaacuucaagagcaacuucgaccuggccgaggacgccaagcugcagcucagcaaggacaccuacgacgacgaccuggacaaucugcuggcccagaucggcgaccaguacgccgaccuguuccuggcugccaagaaucucagcgacgccauccugcucagcgacauccugcgggugaacacagagaucacgaaggccccccucagcgccagcaugaucaagcgguacgacgagcaccaccaggaccugacgcugcugaaggcacuggugcggcagcagcuuccagagaaguacaaggagaucuucuucgaccagagcaagaauggcuacgccggcuacaucgacgguggugccagccaggaggaguucuacaaguucaucaagcccauccuggagaagauggacggcacagaggagcugcuggugaagcugaacagggaggaccugcugcggaagcagcggacguucgacaauggcagcaucccccaccagauccaccugggugagcugcacgccauccugcggcggcaggaggacuucuaccccuuccugaaggacaacagggagaagaucgagaagauccugacguuccggauccccuacuacguuggcccccuggcccggggcaacagccgguucgccuggaugacgcggaagagcgaggagacgaucacucccuggaacuucgaggaagugguggacaagggugccagcgcccagagcuucaucgagcggaugacgaacuucgacaagaaucuucccaacgagaaggugcuucccaagcacagccugcuguacgaguacuucacgguguacaacgagcugacgaaggugaaguacgugacagagggcaugcggaagcccgccuuccucagcggugagcagaagaaggccaucguggaccugcuguucaagacgaaccggaaggugacggugaagcagcugaaggaggacuacuucaagaagaucgagugcuucgacagcguggagaucagcggcguggaggaccgguucaacgccagccugggcaccuaccacgaccugcugaagaucaucaaggacaaggacuuccuggacaacgaggagaacgaggacauccuggaggacaucgugcugacgcugacgcuguucgaggacagggagaugaucgaggagcggcugaagaccuacgcccaccuguucgacgacaaggugaugaagcagcugaagcggcggcgguacacgggcuggggccggcucagccggaagcugaucaauggcauccgagacaagcagagcggcaagacgauccuggacuuccugaagagcgacggcuucgccaaccggaacuucaugcagcugauccacgacgacagccugacguucaaggaggacauccagaaggcccaggucagcggccagggcgacagccugcacgagcacaucgccaaucuggccggcagccccgccaucaagaagggcauccugcagacggugaaggugguggacgagcuggugaaggugaugggccggcacaagccagagaacaucgugaucgagauggccagggagaaccagacgacucagaagggccagaagaacagcagggagcggaugaagcggaucgaggagggcaucaaggagcugggcagccagauccugaaggagcaccccguggagaacacucagcugcagaacgagaagcuguaccuguacuaccugcagaauggccgagacauguacguggaccaggagcuggacaucaaccggcucagcgacuacgacguggaccacaucguuccccagagcuuccugaaggacgacagcaucgacaacaaggugcugacgcggagcgacaagaaccggggcaagagcgacaacguucccagcgaggaaguggugaagaagaugaagaacuacuggcggcagcugcugaacgccaagcugaucacucagcggaaguucgacaaucugacgaaggccgagcgggguggccucagcgagcuggacaaggccggcuucaucaagcggcagcugguggagacgcggcagaucacgaagcacguggcccagauccuggacagccggaugaacacgaaguacgacgagaacgacaagcugaucagggaagugaaggugaucacgcugaagagcaagcuggucagcgacuuccggaaggacuuccaguucuacaaggugagggagaucaacaacuaccaccacgcccacgacgccuaccugaacgcugugguuggcacggcacugaucaagaaguaccccaagcuggagagcgaguucguguacggcgacuacaagguguacgacgugcggaagaugaucgccaagagcgagcaggagaucggcaaggccacggccaaguacuucuucuacagcaacaucaugaacuucuucaagacagagaucacgcuggccaauggugagauccggaagcggccccugaucgagacgaauggugagacgggugagaucgugugggacaagggccgagacuucgccacggugcggaaggugcucagcaugccccaggugaacaucgugaagaagacagaagugcagacggguggcuucagcaaggagagcauccuucccaagcggaacagcgacaagcugaucgcccggaagaaggacugggaccccaagaaguacgguggcuucgacagccccacgguggccuacagcgugcuggugguggccaagguggagaagggcaagagcaagaagcugaagagcgugaaggagcugcugggcaucacgaucauggagcggagcagcuucgagaagaaccccaucgacuuccuggaagccaagggcuacaaggaagugaagaaggaccugaucaucaagcuucccaaguacagccuguucgagcuggagaauggccggaagcggaugcuggccagcgccggugagcugcagaagggcaacgagcuggcacuucccagcaaguacgugaacuuccuguaccuggccagccacuacgagaagcugaagggcagcccagaggacaacgagcagaagcagcuguucguggagcagcacaagcacuaccuggacgagaucaucgagcagaucagcgaguucagcaagcgggugauccuggccgacgccaaucuggacaaggugcucagcgccuacaacaagcaccgagacaagcccaucagggagcaggccgagaacaucauccaccuguucacgcugacgaaucugggugcccccgcugccuucaaguacuucgacacgacgaucgaccggaagcgguacacgucgacgaaggaagugcuggacgccacgcugauccaccagagcaucacgggccuguacgagacgcggaucgaccucagccagcuggguggcgacggugguggcagccccaagaagaagcggaagguguagcuagcaccagccucaagaacacccgaauggagucucuaagcuacauaauaccaacuuacacuuuacaaaauguugucccccaaaauguagccauucguaucugcuccuaauaaaaagaaaguuucuucacauucucucgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaucuag

SEQ ID NO: 21 - RNA, 4430 nt, Artificial Sequence
  Synthetic: Cas9 mRNA transcript comprising SEQ 11
gggaagcucagaauaaacgcucaacuuuggccggaucugccaccauggacaaaaaauacucaauaggccucgacauaggcaccaacucagucggcugggccgucauaaccgacgaguacaaaguccccucaaaaaaauucaaaguccucggcaacaccgacaggcacucaauaaaaaaaaaccucauaggcgcccuccucuucgacucaggcgagaccgccgaggccaccaggcucaaaaggaccgccaggaggagguacaccaggaggaaaaacaggauaugcuaccuccaggagauauucucaaacgagauggccaaagucgacgacucauucuuccacaggcucgaggagucauuccucgucgaggaggacaaaaaacacgagaggcaccccauauucggcaacauagucgacgaggucgccuaccacgagaaauaccccaccauauaccaccucaggaaaaaacucgucgacucaaccgacaaagccgaccucaggcucauauaccucgcccucgcccacaugauaaaauucaggggccacuuccucauagagggcgaccucaaccccgacaacucagacgucgacaaacucuucauacagcucguccagaccuacaaccagcucuucgaggagaaccccauaaacgccucaggcgucgacgccaaagccauacucucagccaggcucucaaaaucaaggaggcucgagaaccucauagcccagcuccccggcgagaaaaaaaacggccucuucggcaaccucauagcccucucacucggccucacccccaacuucaaaucaaacuucgaccucgccgaggacgccaaacuccagcucucaaaagacaccuacgacgacgaccucgacaaccuccucgcccagauaggcgaccaguacgccgaccucuuccucgccgccaaaaaccucucagacgccauacuccucucagacauacucagggucaacaccgagauaaccaaagccccccucucagccucaaugauaaaaagguacgacgagcaccaccaggaccucacccuccucaaagcccucgucaggcagcagcuccccgagaaauacaaagagauauucuucgaccagucaaaaaacggcuacgccggcuacauagacggcggcgccucacaggaggaguucuacaaauucauaaaacccauacucgagaaaauggacggcaccgaggagcuccucgucaaacucaacagggaggaccuccucaggaaacagaggaccuucgacaacggcucaauaccccaccagauacaccucggcgagcuccacgccauacucaggaggcaggaggacuucuaccccuuccucaaagacaacagggagaaaauagagaaaauacucaccuucaggauacccuacuacgucggcccccucgccaggggcaacucaagguucgccuggaugaccaggaaaucagaggagaccauaacccccuggaacuucgaggaggucgucgacaaaggcgccucagcccagucauucauagagaggaugaccaacuucgacaaaaaccuccccaacgagaaaguccuccccaaacacucacuccucuacgaguacuucaccgucuacaacgagcucaccaaagucaaauacgucaccgagggcaugaggaaacccgccuuccucucaggcgagcagaaaaaagccauagucgaccuccucuucaaaaccaacaggaaagucaccgucaaacagcucaaagaggacuacuucaaaaaaauagagugcuucgacucagucgagauaucaggcgucgaggacagguucaacgccucacucggcaccuaccacgaccuccucaaaauaauaaaagacaaagacuuccucgacaacgaggagaacgaggacauacucgaggacauaguccucacccucacccucuucgaggacagggagaugauagaggagaggcucaaaaccuacgcccaccucuucgacgacaaagucaugaaacagcucaaaaggaggagguacaccggcuggggcaggcucucaaggaaacucauaaacggcauaagggacaaacagucaggcaaaaccauacucgacuuccucaaaucagacggcuucgccaacaggaacuucaugcagcucauacacgacgacucacucaccuucaaagaggacauacagaaagcccaggucucaggccagggcgacucacuccacgagcacauagccaaccucgccggcucacccgccauaaaaaaaggcauacuccagaccgucaaagucgucgacgagcucgucaaagucaugggcaggcacaaacccgagaacauagucauagagauggccagggagaaccagaccacccagaaaggccagaaaaacucaagggagaggaugaaaaggauagaggagggcauaaaagagcucggcucacagauacucaaagagcaccccgucgagaacacccagcuccagaacgagaaacucuaccucuacuaccuccagaacggcagggacauguacgucgaccaggagcucgacauaaacaggcucucagacuacgacgucgaccacauagucccccagucauuccucaaagacgacucaauagacaacaaaguccucaccaggucagacaaaaacaggggcaaaucagacaacguccccucagaggaggucgucaaaaaaaugaaaaacuacuggaggcagcuccucaacgccaaacucauaacccagaggaaauucgacaaccucaccaaagccgagaggggcggccucucagagcucgacaaagccggcuucauaaaaaggcagcucgucgagaccaggcagauaaccaaacacgucgcccagauacucgacucaaggaugaacaccaaauacgacgagaacgacaaacucauaagggaggucaaagucauaacccucaaaucaaaacucgucucagacuucaggaaagacuuccaguucuacaaagucagggagauaaacaacuaccaccacgcccacgacgccuaccucaacgccgucgucggcaccgcccucauaaaaaaauaccccaaacucgagucagaguucgucuacggcgacuacaaagucuacgacgucaggaaaaugauagccaaaucagagcaggagauaggcaaagccaccgccaaauacuucuucuacucaaacauaaugaacuucuucaaaaccgagauaacccucgccaacggcgagauaaggaaaaggccccucauagagaccaacggcgagaccggcgagauagucugggacaaaggcagggacuucgccaccgucaggaaaguccucucaaugccccaggucaacauagucaaaaaaaccgagguccagaccggcggcuucucaaaagagucaauacuccccaaaaggaacucagacaaacucauagccaggaaaaaagacugggaccccaaaaaauacggcggcuucgacucacccaccgucgccuacucaguccucgucgucgccaaagucgagaaaggcaaaucaaaaaaacucaaaucagucaaagagcuccucggcauaaccauaauggagaggucaucauucgagaaaaaccccauagacuuccucgaggccaaaggcuacaaagaggucaaaaaagaccucauaauaaaacuccccaaauacucacucuucgagcucgagaacggcaggaaaaggaugcucgccucagccggcgagcuccagaaaggcaacgagcucgcccuccccucaaaauacgucaacuuccucuaccucgccucacacuacgagaaacucaaaggcucacccgaggacaacgagcagaaacagcucuucgucgagcagcacaaacacuaccucgacgagauaauagagcagauaucagaguucucaaaaagggucauacucgccgacgccaaccucgacaaaguccucucagccuacaacaaacacagggacaaacccauaagggagcaggccgagaacauaauacaccucuucacccucaccaaccucggcgcccccgccgccuucaaauacuucgacaccaccauagacaggaaaagguacaccucaaccaaagagguccucgacgccacccucauacaccagucaauaaccggccucuacgagaccaggauagaccucucacagcucggcggcgacggcggcggcucacccaaaaaaaaaaggaaagucuagcuagcaccagccucaagaacacccgaauggagucucuaagcuacauaauaccaacuuacacuuuacaaaauguugucccccaaaauguagccauucguaucugcuccuaauaaaaagaaaguuucuucacauucucucgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaucuag

SEQ ID NO: 22 - RNA, 4427 nt, Artificial Sequence
  Synthetic: Cas9 mRNA transcript comprising SEQ 12
gggaagcucagaauaaacgcucaacuuuggccggaucugccaccauggacaaaaaauacagcaucggccuggacaucggcacgaacagcgugggcugggccgugaucacggacgaguacaaagugcccagcaaaaaauucaaagugcugggcaacacggaccggcacagcaucaaaaaaaaccugaucggcgcccugcuguucgacagcggcgagacggccgaggccacgcggcugaaacggacggcccggcggcgguacacgcggcggaaaaaccggaucugcuaccugcaggagaucuucagcaacgagauggccaaaguggacgacagcuucuuccaccggcuggaggagagcuuccugguggaggaggacaaaaaacacgagcggcaccccaucuucggcaacaucguggacgagguggccuaccacgagaaauaccccacgaucuaccaccugcggaaaaaacugguggacagcacggacaaagccgaccugcggcugaucuaccuggcccuggcccacaugaucaaauuccggggccacuuccugaucgagggcgaccugaaccccgacaacagcgacguggacaaacuguucauccagcuggugcagacguacaaccagcuguucgaggagaaccccaucaacgccagcggcguggacgccaaagccauccugagcgcccggcugagcaaaagccggcggcuggagaaccugaucgcccagcugcccggcgagaaaaaaaacggccuguucggcaaccugaucgcccugagccugggccugacgcccaacuucaaaagcaacuucgaccuggccgaggacgccaaacugcagcugagcaaagacacguacgacgacgaccuggacaaccugcuggcccagaucggcgaccaguacgccgaccuguuccuggccgccaaaaaccugagcgacgccauccugcugagcgacauccugcgggugaacacggagaucacgaaagccccccugagcgccagcaugaucaaacgguacgacgagcaccaccaggaccugacgcugcugaaagcccuggugcggcagcagcugcccgagaaauacaaagagaucuucuucgaccagagcaaaaacggcuacgccggcuacaucgacggcggcgccagccaggaggaguucuacaaauucaucaaacccauccuggagaaaauggacggcacggaggagcugcuggugaaacugaaccgggaggaccugcugcggaaacagcggacguucgacaacggcagcaucccccaccagauccaccugggcgagcugcacgccauccugcggcggcaggaggacuucuaccccuuccugaaagacaaccgggagaaaaucgagaaaauccugacguuccggauccccuacuacgugggcccccuggcccggggcaacagccgguucgccuggaugacgcggaaaagcgaggagacgaucacgcccuggaacuucgaggaggugguggacaaaggcgccagcgcccagagcuucaucgagcggaugacgaacuucgacaaaaaccugcccaacgagaaagugcugcccaaacacagccugcuguacgaguacuucacgguguacaacgagcugacgaaagugaaauacgugacggagggcaugcggaaacccgccuuccugagcggcgagcagaaaaaagccaucguggaccugcuguucaaaacgaaccggaaagugacggugaaacagcugaaagaggacuacuucaaaaaaaucgagugcuucgacagcguggagaucagcggcguggaggaccgguucaacgccagccugggcacguaccacgaccugcugaaaaucaucaaagacaaagacuuccuggacaacgaggagaacgaggacauccuggaggacaucgugcugacgcugacgcuguucgaggaccgggagaugaucgaggagcggcugaaaacguacgcccaccuguucgacgacaaagugaugaaacagcugaaacggcggcgguacacgggcuggggccggcugagccggaaacugaucaacggcauccgggacaaacagagcggcaaaacgauccuggacuuccugaaaagcgacggcuucgccaaccggaacuucaugcagcugauccacgacgacagccugacguucaaagaggacauccagaaagcccaggugagcggccagggcgacagccugcacgagcacaucgccaaccuggccggcagccccgccaucaaaaaaggcauccugcagacggugaaagugguggacgagcuggugaaagugaugggccggcacaaacccgagaacaucgugaucgagauggcccgggagaaccagacgacgcagaaaggccagaaaaacagccgggagcggaugaaacggaucgaggagggcaucaaagagcugggcagccagauccugaaagagcaccccguggagaacacgcagcugcagaacgagaaacuguaccuguacuaccugcagaacggccgggacauguacguggaccaggagcuggacaucaaccggcugagcgacuacgacguggaccacaucgugccccagagcuuccugaaagacgacagcaucgacaacaaagugcugacgcggagcgacaaaaaccggggcaaaagcgacaacgugcccagcgaggagguggugaaaaaaaugaaaaacuacuggcggcagcugcugaacgccaaacugaucacgcagcggaaauucgacaaccugacgaaagccgagcggggcggccugagcgagcuggacaaagccggcuucaucaaacggcagcugguggagacgcggcagaucacgaaacacguggcccagauccuggacagccggaugaacacgaaauacgacgagaacgacaaacugauccgggaggugaaagugaucacgcugaaaagcaaacuggugagcgacuuccggaaagacuuccaguucuacaaagugcgggagaucaacaacuaccaccacgcccacgacgccuaccugaacgccguggugggcacggcccugaucaaaaaauaccccaaacuggagagcgaguucguguacggcgacuacaaaguguacgacgugcggaaaaugaucgccaaaagcgagcaggagaucggcaaagccacggccaaauacuucuucuacagcaacaucaugaacuucuucaaaacggagaucacgcuggccaacggcgagauccggaaacggccccugaucgagacgaacggcgagacgggcgagaucgugugggacaaaggccgggacuucgccacggugcggaaagugcugagcaugccccaggugaacaucgugaaaaaaacggaggugcagacgggcggcuucagcaaagagagcauccugcccaaacggaacagcgacaaacugaucgcccggaaaaaagacugggaccccaaaaaauacggcggcuucgacagccccacgguggccuacagcgugcuggugguggccaaaguggagaaaggcaaaagcaaaaaacugaaaagcgugaaagagcugcugggcaucacgaucauggagcggagcagcuucgagaaaaaccccaucgacuuccuggaggccaaaggcuacaaagaggugaaaaaagaccugaucaucaaacugcccaaauacagccuguucgagcuggagaacggccggaaacggaugcuggccagcgccggcgagcugcagaaaggcaacgagcuggcccugcccagcaaauacgugaacuuccuguaccuggccagccacuacgagaaacugaaaggcagccccgaggacaacgagcagaaacagcuguucguggagcagcacaaacacuaccuggacgagaucaucgagcagaucagcgaguucagcaaacgggugauccuggccgacgccaaccuggacaaagugcugagcgccuacaacaaacaccgggacaaacccauccgggagcaggccgagaacaucauccaccuguucacgcugacgaaccugggcgcccccgccgccuucaaauacuucgacacgacgaucgaccggaaacgguacacgagcacgaaagaggugcuggacgccacgcugauccaccagagcaucacgggccuguacgagacgcggaucgaccugagccagcugggcggcgacggcggcggcagccccaaaaaaaaacggaaaguguagcuagcaccagccucaagaacacccgaauggagucucuaagcuacauaauaccaacuuacacuuuacaaaauguugucccccaaaauguagccauucguaucugcuccuaauaaaaagaaaguuucuucacauucucucgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaucuag

SEQ ID NO: 23 - RNA, 4424 nt, Artificial Sequence
  Synthetic: Cas9 transcript comprising SEQ 13
gggaagcucagaauaaacgcucaacuuuggccggaucugccaccauggauaaaaaauauucaauaggauuagauauaggaacaaauucaguaggaugggcaguaauaacagaugaauauaaaguaccaucaaaaaaauuuaaaguauuaggaaauacagauagacauucaauaaaaaaaaauuuaauaggagcauuauuauuugauucaggagaaacagcagaagcaacaagauuaaaaagaacagcaagaagaagauauacaagaagaaaaaauagaauauguuauuuacaagaaauauuuucaaaugaaauggcaaaaguagaugauucauuuuuucauagauuagaagaaucauuuuuaguagaagaagauaaaaaacaugaaagacauccaauauuuggaaauauaguagaugaaguagcauaucaugaaaaauauccaacaauauaucauuuaagaaaaaaauuaguagauucaacagauaaagcagauuuaagauuaauauauuuagcauuagcacauaugauaaaauuuagaggacauuuuuuaauagaaggagauuuaaauccagauaauucagauguagauaaauuauuuauacaauuaguacaaacauauaaucaauuauuugaagaaaauccaauaaaugcaucaggaguagaugcaaaagcaauauuaucagcaagauuaucaaaaucaagaagauuagaaaauuuaauagcacaauuaccaggagaaaaaaaaaauggauuauuuggaaauuuaauagcauuaucauuaggauuaacaccaaauuuuaaaucaaauuuugauuuagcagaagaugcaaaauuacaauuaucaaaagauacauaugaugaugauuuagauaauuuauuagcacaaauaggagaucaauaugcagauuuauuuuuagcagcaaaaaauuuaucagaugcaauauuauuaucagauauauuaagaguaaauacagaaauaacaaaagcaccauuaucagcaucaaugauaaaaagauaugaugaacaucaucaggacuuaacauuauuaaaagcauuaguaagacaacaauuaccagaaaaauauaaagaaauauuuuuugaucaaucaaaaaauggauaugcaggauauauagauggaggagcaucacaagaagaauuuuauaaauuuauaaaaccaauauuagaaaaaauggauggaacagaagaauuauuaguaaaauuaaauagagaagauuuauuaagaaaacaaagaacauuugauaauggaucaauaccacaucaaauacauuuaggagaauuacaugcaauauuaagaagacaagaagauuuuuauccauuuuuaaaagauaauagagaaaaaauagaaaaaauauuaacauuuagaauaccauauuauguaggaccauuagcaagaggaaauucaagauuugcauggaugacaagaaaaucagaagaaacaauaacaccauggaauuuugaagaaguaguagauaaaggagcaucagcacaaucauuuauagaaagaaugacaaauuuugauaaaaauuuaccaaaugaaaaaguauuaccaaaacauucauuauuauaugaauauuuuacaguauauaaugaauuaacaaaaguaaaauauguaacagaaggaaugagaaaaccagcauuuuuaucaggagaacaaaaaaaagcaauaguagauuuauuauuuaaaacaaauagaaaaguaacaguaaaacaauuaaaagaagauuauuuuaaaaaaauagaauguuuugauucaguagaaauaucaggaguagaagauagauuuaaugcaucauuaggaacauaucaugauuuauuaaaaauaauaaaagauaaagauuuuuuagauaaugaagaaaaugaagauauauuagaagauauaguauuaacauuaacauuauuugaagauagagaaaugauagaagaaagauuaaaaacauaugcacauuuauuugaugauaaaguaaugaaacaauuaaaaagaagaagauauacaggauggggaagauuaucaagaaaauuaauaaauggaauaagagauaaacaaucaggaaaaacaauauuagauuuuuuaaaaucagauggauuugcaaauagaaauuuuaugcaauuaauacaugaugauucauuaacauuuaaagaagauauacaaaaagcacaaguaucaggacaaggagauucauuacaugaacauauagcaaauuuagcaggaucaccagcaauaaaaaaaggaauauuacaaacaguaaaaguaguagaugaauuaguaaaaguaaugggaagacauaaaccagaaaauauaguaauagaaauggcaagagaaaaucaaacaacacaaaaaggacaaaaaaauucaagagaaagaaugaaaagaauagaagaaggaauaaaagaauuaggaucacaaauauuaaaagaacauccaguagaaaauacacaauuacaaaaugaaaaauuauauuuauauuauuuacaaaauggaagagauauguauguagaucaagaauuagauauaaauagauuaucagauuaugauguagaucauauaguaccacaaucauuuuuaaaagaugauucaauagauaauaaaguauuaacaagaucagauaaaaauagaggaaaaucagauaauguaccaucagaagaaguaguaaaaaaaaugaaaaauuauuggagacaauuauuaaaugcaaaauuaauaacacaaagaaaauuugauaauuuaacaaaagcagaaagaggaggauuaucagaauuagauaaagcaggauuuauaaaaagacaauuaguagaaacaagacaaauaacaaaacauguagcacaaauauuagauucaagaaugaauacaaaauaugaugaaaaugauaaauuaauaagagaaguaaaaguaauaacauuaaaaucaaaauuaguaucagauuuuagaaaagauuuucaauuuuauaaaguaagagaaauaaauaauuaucaucaugcacaugaugcauauuuaaaugcaguaguaggaacagcauuaauaaaaaaauauccaaaauuagaaucagaauuuguauauggagauuauaaaguauaugauguaagaaaaaugauagcaaaaucagaacaagaaauaggaaaagcaacagcaaaauauuuuuuuuauucaaauauaaugaauuuuuuuaaaacagaaauaacauuagcaaauggagaaauaagaaaaagaccauuaauagaaacaaauggagaaacaggagaaauaguaugggauaaaggaagagauuuugcaacaguaagaaaaguauuaucaaugccacaaguaaauauaguaaaaaaaacagaaguacaaacaggaggauuuucaaaagaaucaauauuaccaaaaagaaauucagauaaauuaauagcaagaaaaaaagauugggauccaaaaaaauauggaggauuugauucaccaacaguagcauauucaguauuaguaguagcaaaaguagaaaaaggaaaaucaaaaaaauuaaaaucaguaaaagaauuauuaggaauaacaauaauggaaagaucaucauuugaaaaaaauccaauagauuuuuuagaagcaaaaggauauaaagaaguaaaaaaagauuuaauaauaaaauuaccaaaauauucauuauuugaauuagaaaauggaagaaaaagaauguuagcaucagcaggagaauuacaaaaaggaaaugaauuagcauuaccaucaaaauauguaaauuuuuuauauuuagcaucacauuaugaaaaauuaaaaggaucaccagaagauaaugaacaaaaacaauuauuuguagaacaacauaaacauuauuuagaugaaauaauagaacaaauaucagaauuuucaaaaagaguaauauuagcagaugcaaauuuagauaaaguauuaucagcauauaauaaacauagagauaaaccaauaagagaacaagcagaaaauauaauacauuuauuuacauuaacaaauuuaggagcaccagcagcauuuaaauauuuugauacaacaauagauagaaaaagauauacaucaacaaaagaaguauuagaugcaacauuaauacaucaaucaauaacaggauuauaugaaacaagaauagauuuaucacaauuaggaggagauggaggaggaucaccaaaaaaaaaaagaaaaguauagcuagcaccagccucaagaacacccgaauggagucucuaagcuacauaauaccaacuuacacuuuacaaaauguugucccccaaaauguagccauucguaucugcuccuaauaaaaagaaaguuucuucacauucucucgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaucuag

SEQ ID NO: 24 - RNA, 4425 nt, Artificial Sequence
  Synthetic: Cas9 transcript comprising SEQ 14
gggaagcucagaauaaacgcucaacuuuggccggaucugccaccauggauaaaaaguacagcaucggauuagauauaggaacaaauucaguuggcugggcugugauaacagaugaauauaaaguucccucaaaaaaauuuaaaguauuaggaaauacagauagacauagcaucaaaaaaaaucucauaggugcacuguuauuugauucaggugagacagcagaagccacaagauuaaaaagaacagcccgcagaagauauacaagaagaaaaaauagaauauguuauuuacaggagauauuuucaaaugaaauggcaaaaguagaugauucauuuuuucauagauuagaagaaucauuccugguagaagaagauaaaaaacaugaaagacauccaauauuuggaaauauaguagaugaagucgcauaucaugaaaaguaccccaccauauaucaucugcggaaaaaauuaguagauucgacugauaaagcagaucugcgguuaauauauuuagcacuggcacauaugauaaaauuuagaggacauuuccugauagaaggagauuuaaauccugacaauucagauguagauaaauuauuuauacaauuaguacaaaccuacaaucaauuauuugaagaaaauccaauaaaugcaucaggaguagaugcaaaagcaauacucagcgcccgccucagcaaaucaagaagauuagaaaaucucauagcacaacuuccaggugagaaaaaaaauggguuauuuggaaaucucauagcacucagcuuaggauuaacucccaauuuuaaaucaaauuuugauuuagcagaagaugcaaaauuacaacucagcaaagauaccuacgaugaugauuuagauaaucucuuagcacaaauaggagaucaauaugcagauuuauuccuggcugccaaaaaucucagcgaugcaauauuacucagcgauauacugcggguaaauacagagauaacaaaagcaccacucagcgcaucaaugauaaaaagauaugaugaacaucaucaagauuuaacauuauuaaaagcacugguaagacaacaacuuccagagaaguacaaagaaauauuuuuugaucagagcaaaaauggguaugccggguauauagaugguggugccucacaggaggaauuuuauaaauuuauaaaaccaauauuagaaaaaauggauggaacagaggagcuguuaguaaaauuaaauagggaggauuuacugcggaaacaaagaacauuugauaaugggagcaucccccaucaaauacauuuaggugagcugcaugcaauacugcggagacaggaggauuuuuauccauuccugaaagauaauagggagaaaauagaaaaaauauuaacauuuagaauccccuauuauguuggcccauuagcccgcggaaauucaagauuugcauggaugacaagaaaaucagaagaaacaauaacucccuggaauuuugaagaagucguagauaagggugccucagcacagagcuuuauagaaagaaugacaaauuuugauaaaaaucuuccaaaugaaaaaguacuuccaaaacauucauuauuauaugaauauuuuacaguauauaaugagcugacaaaaguaaaguacguaacagagggaaugagaaaaccagcauuccucagcggugagcaaaaaaaagcaauaguagauuuauuauuuaaaacaaauagaaaaguaacaguaaaacaauuaaaagaagauuauuuuaaaaaaauagaauguuuugauucaguagaaauaucaggaguagaagauagauuuaaugcaucauuaggaaccuaccaugauuuauuaaaaauaauaaaagauaaagauuuccuggauaaugaagaaaaugaagauauauuagaagauauaguauuaacauuaacauuauuugaagauagggagaugauagaagaaagauuaaaaaccuacgcacauuuauuugaugauaaaguaaugaaacaauuaaaaagaagaagauauacaggauggggaagacucagcagaaaauuaauaaaugggauacgagacaaacagagcggaaaaacaauauuagauuuccugaaaucagauggauuugcaaauagaaauuuuaugcaauuaauacaugaugauucauuaacauuuaaagaagauauacaaaaagcacaggucagcggacagggcgauucauuacaugaacauauagcaaaucucgccgggucaccagcaauaaaaaaggggauauuacaaacaguaaaaguaguagaugagcugguaaaaguaaugggaagacauaaaccagagaauauaguaauagaaauggccagggagaaucaaacaacucaaaaggggcaaaaaaauucaagggagagaaugaaaagaauagaagaaggaauaaaagagcugggaucacaaauauuaaaagaacauccaguagaaaauacucaauuacaaaaugaaaaauuauauuuauauuauuuacaaaaugggcgagacauguauguagaucaggagcuggauauaaauagacucagcgauuaugauguagaucauauaguuccccagagcuuccugaaagaugauagcaucgauaauaaaguauuaacaagaucagauaaaaauagaggaaaaucagauaauguucccucagaagaagucguaaaaaaaaugaaaaauuauuggagacaauuauuaaaugcaaaauuaauaacucaaagaaaauuugauaaucucacaaaagcagaaagagguggccucagcgagcuggauaaagccggguuuauaaaaagacaauuaguagaaacaagacaaauaacaaaacauguagcacaaauauuagauucaagaaugaauacaaaguacgaugaaaaugauaaauuaauaagggaagucaaaguaauaacauuaaaaucaaaauuagucagcgauuuuagaaaagauuuucaauuuuauaaaguaagggagauaaauaauuaucaucaugcacaugaugcauauuuaaaugcugugguuggcacagcacugauaaaaaaguacccaaaauuagaaucagaauuuguauauggagauuauaaaguauaugauguaagaaaaaugauagcaaaaucagaacaggagauaggaaaagcaacagcaaaguacuuuuuuuauucaaauauaaugaauuuuuuuaaaacagagauaacauuagcaaauggugagauaagaaaaagaccauuaauagaaacaaauggugagacaggugagauaguaugggauaaggggcgagacuuugcaacaguaagaaaaguacucagcaugccacaggugaauauaguaaaaaaaacagaaguccaaacagguggcuuuucaaaagaaagcauccuuccaaaaagaaauucagauaaauuaauagcccgcaaaaaagauugggauccaaaaaaguacgguggcuuugauucacccaccguagcauauucaguauuaguaguagcaaaaguagaaaaggggaaaucaaaaaaauuaaaaucaguaaaagagcuguuaggaauaacaauaauggaaagaucaucauuugaaaaaaauccaauagauuuccuggaagccaagggguauaaagaagucaaaaaagauuuaauaauaaaacuuccaaaguacucauuauuugagcuggaaaaugggagaaaaagaauguuagcaucagccggugagcugcaaaaggggaaugagcuggcacuucccucaaaguacguaaauuuccuguauuuagcaucacauuaugaaaaauuaaaggggucaccagaggauaaugaacaaaaacaauuauuuguagaacaacauaaacauuauuuagaugaaauaauagaacaaauaucagaauuuucaaaaagaguaauauuagcagaugcaaaucucgauaaaguacucagcgcauauaauaaacaucgagacaaaccaauaagggagcaggccgaaaauauaauacauuuauuuacauuaacaaaucucggugccccagcugccuuuaaguacuuugauacaacaauagauagaaaaagauauacaucgacuaaagaagucuuagaugcaacauuaauacaucagagcaucacaggauuauaugaaacaagaauagaucucagccaauuagguggcgauggugguggcucaccaaaaaaaaaaagaaaaguauagcuagcaccagccucaagaacacccgaauggagucucuaagcuacauaauaccaacuuacacuuuacaaaauguugucccccaaaauguagccauucguaucugcuccuaauaaaaagaaaguuucuucacauucucucgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaucuag

SEQ ID NO: 25-28 - skipped (4 entries)

SEQ ID NO: 29 - RNA, 4140 nt, Artificial Sequence
  Synthetic: E-pair enriched, Table 6 codon enriched Cas9 ORF
auggacaagaaguacagcaucggacuggacaucggaacaaacagcguuggcugggcugugaucacagacgaauacaagguucccucaaagaaguucaagguccugggaaacacagacagacacagcaucaagaagaaucucaucggugcacugcuguucgacagcggugagacagcagaagccacaagacugaagagaacagcccgcagaagauacacaagaagaaagaacagaaucugcuaccugcaggagaucuucagcaacgaaauggcaaaggucgacgacagcuucuuccacagacuggaagaaagcuuccuggucgaagaagacaagaagcacgaaagacacccgaucuucggaaacaucgucgacgaagucgcauaccacgaaaaguaccccaccaucuaccaccugcggaagaagcuggucgacucgacugacaaggcagaccugcggcugaucuaccuggcacuggcacacaugauaaaguucagaggacacuuccugaucgaaggagaccugaacccugacaacagcgacgucgacaagcuguucauccagcugguccagaccuacaaccagcuguucgaagaaaacccgaucaacgcaagcggagucgacgcaaaggcaauccucagcgcccgccucagcaagagcagaagacuggaaaaucucaucgcacagcuuccaggugagaagaagaaugggcuguucggaaaucucaucgcacucagccugggacugacucccaacuucaagagcaacuucgaccuggcagaagacgcaaagcugcagcucagcaaggacaccuacgacgacgaccuggacaaucuccuggcacagaucggagaccaguacgcagaccuguuccuggcugccaagaaucucagcgacgcaauccugcucagcgacauccugcgggucaacacagagaucacaaaggcaccgcucagcgcaagcaugauaaagagauacgacgaacaccaccaggaccugacacugcugaaggcacuggucagacagcagcuuccagagaaguacaaggaaaucuucuucgaccagagcaagaauggguacgccggguacaucgacgguggugccagccaggaggaauucuacaaguucaucaagccgauccuggaaaagauggacggaacagaggagcugcuggucaagcugaacagggaggaccugcugcggaagcagagaacauucgacaaugggagcaucccccaccagauccaccugggugagcugcacgcaauccugcggagacaggaggacuucuacccguuccugaaggacaacagggagaagaucgaaaagauccugacauucagaauccccuacuacguuggcccgcuggcccgcggaaacagcagauucgcauggaugacaagaaagagcgaagaaacaaucacucccuggaacuucgaagaagucgucgacaagggugccagcgcacagagcuucaucgaaagaaugacaaacuucgacaagaaucuuccaaacgaaaagguccuuccaaagcacagccugcuguacgaauacuucacagucuacaacgagcugacaaaggucaaguacgucacagagggaaugagaaagccggcauuccucagcggugagcagaagaaggcaaucgucgaccugcuguucaagacaaacagaaaggucacagucaagcagcugaaggaagacuacuucaagaagaucgaaugcuucgacagcgucgaaaucagcggagucgaagacagauucaacgcaagccugggaaccuaccacgaccugcugaagaucaucaaggacaaggacuuccuggacaacgaagaaaacgaagacauccuggaagacaucguccugacacugacacuguucgaagacagggagaugauagaagaaagacugaagaccuacgcacaccuguucgacgacaaggucaugaagcagcugaagagaagaagauacacaggauggggaagacucagcagaaagcugaucaaugggauccgagacaagcagagcggaaagacaauccuggacuuccugaagagcgacggauucgcaaacagaaacuucaugcagcugauccacgacgacagccugacauucaaggaagacauccagaaggcacaggucagcggacagggcgacagccugcacgaacacaucgcaaaucucgccgggagcccggcaaucaagaaggggauccugcagacagucaaggucgucgacgagcuggucaaggucaugggaagacacaagccagagaacaucgucaucgaaauggccagggagaaccagacaacucaaaaggggcagaagaacagcagggagagaaugaagagaaucgaagaaggaaucaaggagcugggaagccagauccugaaggaacacccggucgaaaacacucaacugcagaacgaaaagcuguaccuguacuaccugcagaaugggcgagacauguacgucgaccaggagcuggacaucaacagacucagcgacuacgacgucgaccacaucguuccccagagcuuccugaaggacgacagcaucgacaacaagguccugacaagaagcgacaagaacagaggaaagagcgacaacguucccucagaagaagucgucaagaagaugaagaacuacuggagacagcugcugaacgcaaagcugaucacucaaagaaaguucgacaaucucacaaaggcagaaagagguggccucagcgagcuggacaaggccggguucaucaagagacagcuggucgaaacaagacagaucacaaagcacgucgcacagauccuggacagcagaaugaacacaaaguacgacgaaaacgacaagcugaucagggaagucaaggucaucacacugaagagcaagcuggucagcgacuucagaaaggacuuccaguucuacaaggucagggagaucaacaacuaccaccacgcacacgacgcauaccugaacgcugugguuggcacagcacugaucaagaaguacccgaagcuggaaagcgaauucgucuacggagacuacaaggucuacgacgucagaaagaugauagcaaagagcgaacaggagaucggaaaggcaacagcaaaguacuucuucuacagcaacaucaugaacuucuucaagacagagaucacacuggcaaauggugagaucagaaagagaccgcugaucgaaacaaauggugagacaggugagaucgucugggacaaggggcgagacuucgcaacagucagaaagguccucagcaugccgcaggugaacaucgucaagaagacagaaguccagacagguggcuucagcaaggaaagcauccuuccaaagagaaacagcgacaagcugaucgcccgcaagaaggacugggacccgaagaaguacgguggcuucgacagccccaccgucgcauacagcguccuggucgucgcaaaggucgaaaaggggaagagcaagaagcugaagagcgucaaggagcugcugggaaucacaaucauggaaagaagcagcuucgaaaagaacccgaucgacuuccuggaagccaagggguacaaggaagucaagaaggaccugaucaucaagcuuccaaaguacagccuguucgagcuggaaaaugggagaaagagaaugcuggcaagcgccggugagcugcagaaggggaacgagcuggcacuucccucaaaguacgucaacuuccuguaccuggcaagccacuacgaaaagcugaaggggagcccagaggacaacgaacagaagcagcuguucgucgaacagcacaagcacuaccuggacgaaaucaucgaacagaucagcgaauucagcaagagagucauccuggcagacgcaaaucucgacaagguccucagcgcauacaacaagcaccgagacaagccgaucagggagcaggccgaaaacaucauccaccuguucacacugacaaaucucggugccccggcugccuucaaguacuucgacacaacaaucgacagaaagagauacacaucgacuaaggaaguccuggacgcaacacugauccaccagagcaucacaggacuguacgaaacaagaaucgaccucagccagcuggguggcgacggugguggcagcccgaagaagaagagaaaggucuag

SEQ ID NO: 30-45 - skipped (16 entries)

SEQ ID NO: 46 - RNA, 4140 nt, Artificial Sequence
  Synthetic: E-pair enriched, Table 7 Low A codon enriched Cas9 ORF
auggacaagaaguacagcaucggccuggacaucggcaccaacuccguuggcugggcugugaucaccgacgaguacaagguucccucaaagaaguucaaggugcugggcaacaccgaccggcacagcaucaagaagaaucucaucggugcacugcuguucgacuccggugagaccgccgaagccacccggcugaagcggaccgcccgccggcgguacacccggcggaagaaccggaucugcuaccugcaggagaucuucuccaacgagauggccaagguggacgacuccuucuuccaccggcuggaggaguccuuccugguggaggaggacaagaagcacgagcggcaccccaucuucggcaacaucguggacgaagucgccuaccacgagaaguaccccaccaucuaccaccugcggaagaagcugguggacucgacugacaaggccgaccugcggcugaucuaccuggcacuggcccacaugauaaaguuccggggccacuuccugaucgagggcgaccugaacccugacaacuccgacguggacaagcuguucauccagcuggugcagaccuacaaccagcuguucgaggagaaccccaucaacgccuccggcguggacgccaaggccauccucagcgcccgccucagcaagucccggcggcuggagaaucucaucgcccagcuuccaggugagaagaagaaugggcuguucggcaaucucaucgcacucagccugggccugacucccaacuucaaguccaacuucgaccuggccgaggacgccaagcugcagcucagcaaggacaccuacgacgacgaccuggacaaucuccuggcccagaucggcgaccaguacgccgaccuguuccuggcugccaagaaucucagcgacgccauccugcucagcgacauccugcgggugaacacagagaucaccaaggccccccucagcgccuccaugauaaagcgguacgacgagcaccaccaggaccugacccugcugaaggcacuggugcggcagcagcuuccagagaaguacaaggagaucuucuucgaccagagcaagaauggguacgccggguacaucgacgguggugccucccaggaggaguucuacaaguucaucaagcccauccuggagaagauggacggcacagaggagcugcuggugaagcugaacagggaggaccugcugcggaagcagcggaccuucgacaaugggagcaucccccaccagauccaccugggugagcugcacgccauccugcggcggcaggaggacuucuaccccuuccugaaggacaacagggagaagaucgagaagauccugaccuuccggauccccuacuacguuggcccccuggcccgcggcaacucccgguucgccuggaugacccggaaguccgaggagaccaucacucccuggaacuucgaggaagucguggacaagggugccuccgcccagagcuucaucgagcggaugaccaacuucgacaagaaucuuccaaacgagaaggugcuuccaaagcacucccugcuguacgaguacuucaccguguacaacgagcugaccaaggugaaguacgugacagagggcaugcggaagcccgccuuccucagcggugagcagaagaaggccaucguggaccugcuguucaagaccaaccggaaggugaccgugaagcagcugaaggaggacuacuucaagaagaucgagugcuucgacuccguggagaucuccggcguggaggaccgguucaacgccucccugggcaccuaccacgaccugcugaagaucaucaaggacaaggacuuccuggacaacgaggagaacgaggacauccuggaggacaucgugcugacccugacccuguucgaggacagggagaugauagaggagcggcugaagaccuacgcccaccuguucgacgacaaggugaugaagcagcugaagcggcggcgguacaccggcuggggccggcucagccggaagcugaucaaugggauccgagacaagcagagcggcaagaccauccuggacuuccugaaguccgacggcuucgccaaccggaacuucaugcagcugauccacgacgacucccugaccuucaaggaggacauccagaaggcccaggucagcggccagggcgacucccugcacgagcacaucgccaaucucgccgggucccccgccaucaagaaggggauccugcagaccgugaaggugguggacgagcuggugaaggugaugggccggcacaagccagagaacaucgugaucgagauggccagggagaaccagaccacucaaaaggggcagaagaacuccagggagcggaugaagcggaucgaggagggcaucaaggagcugggcucccagauccugaaggagcaccccguggagaacacucaacugcagaacgagaagcuguaccuguacuaccugcagaaugggcgagacauguacguggaccaggagcuggacaucaaccggcucagcgacuacgacguggaccacaucguuccccagagcuuccugaaggacgacagcaucgacaacaaggugcugacccgguccgacaagaaccggggcaaguccgacaacguucccucagaggaagucgugaagaagaugaagaacuacuggcggcagcugcugaacgccaagcugaucacucaacggaaguucgacaaucucaccaaggccgagcgggguggccucagcgagcuggacaaggccggguucaucaagcggcagcugguggagacccggcagaucaccaagcacguggcccagauccuggacucccggaugaacaccaaguacgacgagaacgacaagcugaucagggaagucaaggugaucacccugaaguccaagcuggucagcgacuuccggaaggacuuccaguucuacaaggugagggagaucaacaacuaccaccacgcccacgacgccuaccugaacgcugugguuggcaccgcacugaucaagaaguaccccaagcuggaguccgaguucguguacggcgacuacaagguguacgacgugcggaagaugauagccaaguccgagcaggagaucggcaaggccaccgccaaguacuucuucuacuccaacaucaugaacuucuucaagacagagaucacccuggccaauggugagauccggaagcggccccugaucgagaccaauggugagaccggugagaucgugugggacaaggggcgagacuucgccaccgugcggaaggugcucagcaugccccaggugaacaucgugaagaagacagaaguccagaccgguggcuucuccaaggagagcauccuuccaaagcggaacuccgacaagcugaucgcccgcaagaaggacugggaccccaagaaguacgguggcuucgacucccccaccguggccuacuccgugcuggugguggccaagguggagaaggggaaguccaagaagcugaaguccgugaaggagcugcugggcaucaccaucauggagcgguccuccuucgagaagaaccccaucgacuuccuggaagccaagggguacaaggaagucaagaaggaccugaucaucaagcuuccaaaguacucccuguucgagcuggagaaugggcggaagcggaugcuggccuccgccggugagcugcagaaggggaacgagcuggcacuucccucaaaguacgugaacuuccuguaccuggccucccacuacgagaagcugaagggguccccagaggacaacgagcagaagcagcuguucguggagcagcacaagcacuaccuggacgagaucaucgagcagaucuccgaguucuccaagcgggugauccuggccgacgccaaucucgacaaggugcucagcgccuacaacaagcaccgagacaagcccaucagggagcaggccgagaacaucauccaccuguucacccugaccaaucucggugcccccgcugccuucaaguacuucgacaccaccaucgaccggaagcgguacaccucgacuaaggaaguccuggacgccacccugauccaccagagcaucaccggccuguacgagacccggaucgaccucagccagcuggguggcgacggugguggcucccccaagaagaagcggaagguguag

SEQ ID NO: 47-66 - skipped (20 entries)

SEQ ID NO: 67 - DNA, 4104 bp, Artificial Sequence
  Synthetic: WT Cas9 ORF
atggataagaagtactcaatcgggctggatatcggaactaattccgtgggttgggcagtgatcacggatgaatacaaagtgccgtccaagaagttcaaggtcctggggaacaccgatagacacagcatcaagaaaaatctcatcggagccctgctgtttgactccggcgaaaccgcagaagcgacccggctcaaacgtaccgcgaggcgacgctacacccggcggaagaatcgcatctgctatctgcaagagatcttttcgaacgaaatggcaaaggtcgacgacagcttcttccaccgcctggaagaatctttcctggtggaggaggacaagaagcatgaacggcatcctatctttggaaacatcgtcgacgaagtggcgtaccacgaaaagtacccgaccatctaccatctgcggaagaagttggttgactcaactgacaaggccgacctcagattgatctacttggccctcgcccatatgatcaaattccgcggacacttcctgatcgaaggcgatctgaaccctgataactccgacgtggataagcttttcattcaactggtgcagacctacaaccaactgttcgaagaaaacccaatcaatgctagcggcgtcgatgccaaggccatcctgtccgcccggctgtcgaagtcgcggcgcctcgaaaacctgatcgcacagctgccgggagagaaaaagaacggacttttcggcaacttgatcgctctctcactgggactcactcccaatttcaagtccaattttgacctggccgaggacgcgaagctgcaactctcaaaggacacctacgacgacgacttggacaatttgctggcacaaattggcgatcagtacgcggatctgttccttgccgctaagaacctttcggacgcaatcttgctgtccgatatcctgcgcgtgaacaccgaaataaccaaagcgccgcttagcgcctcgatgattaagcggtacgacgagcatcaccaggatctcacgctgctcaaagcgctcgtgagacagcaactgcctgaaaagtacaaggagatcttcttcgaccagtccaagaatgggtacgcagggtacatcgatggaggcgctagccaggaagagttctataagttcatcaagccaatcctggaaaagatggacggaaccgaagaactgctggtcaagctgaacagggaggatctgctccggaaacagagaacctttgacaacggatccattccccaccagatccatctgggtgagctgcacgccatcttgcggcgccaggaggacttttacccattcctcaaggacaaccgggaaaagatcgagaaaattctgacgttccgcatcccgtattacgtgggcccactggcgcgcggcaattcgcgcttcgcgtggatgactagaaaatcagaggaaaccatcactccttggaatttcgaggaagttgtggataagggagcttcggcacaaagcttcatcgaacgaatgaccaacttcgacaagaatctcccaaacgagaaggtgcttcctaagcacagcctcctttacgaatacttcactgtctacaacgaactgactaaagtgaaatacgttactgaaggaatgaggaagccggcctttctgtccggagaacagaagaaagcaattgtcgatctgctgttcaagaccaaccgcaaggtgaccgtcaagcagcttaaagaggactacttcaagaagatcgagtgtttcgactcagtggaaatcagcggggtggaggacagattcaacgcttcgctgggaacctatcatgatctcctgaagatcatcaaggacaaggacttccttgacaacgaggagaacgaggacatcctggaagatatcgtcctgaccttgacccttttcgaggatcgcgagatgatcgaggagaggcttaagacctacgctcatctcttcgacgataaggtcatgaaacaactcaagcgccgccggtacactggttggggccgcctctcccgcaagctgatcaacggtattcgcgataaacagagcggtaaaactatcctggatttcctcaaatcggatggcttcgctaatcgtaacttcatgcaattgatccacgacgacagcctgacctttaaggaggacatccaaaaagcacaagtgtccggacagggagactcactccatgaacacatcgcgaatctggccggttcgccggcgattaagaagggaattctgcaaactgtgaaggtggtcgacgagctggtgaaggtcatgggacggcacaaaccggagaatatcgtgattgaaatggcccgagaaaaccagactacccagaagggccagaaaaactcccgcgaaaggatgaagcggatcgaagaaggaatcaaggagctgggcagccagatcctgaaagagcacccggtggaaaacacgcagctgcagaacgagaagctctacctgtactatttgcaaaatggacgggacatgtacgtggaccaagagctggacatcaatcggttgtctgattacgacgtggaccacatcgttccacagtcctttctgaaggatgactcgatcgataacaaggtgttgactcgcagcgacaagaacagagggaagtcagataatgtgccatcggaggaggtcgtgaagaagatgaagaattactggcggcagctcctgaatgcgaagctgattacccagagaaagtttgacaatctcactaaagccgagcgcggcggactctcagagctggataaggctggattcatcaaacggcagctggtcgagactcggcagattaccaagcacgtggcgcagatcttggactcccgcatgaacactaaatacgacgagaacgataagctcatccgggaagtgaaggtgattaccctgaaaagcaaacttgtgtcggactttcggaaggactttcagttttacaaagtgagagaaatcaacaactaccatcacgcgcatgacgcatacctcaacgctgtggtcggtaccgccctgatcaaaaagtaccctaaacttgaatcggagtttgtgtacggagactacaaggtctacgacgtgaggaagatgatagccaagtccgaacaggaaatcgggaaagcaactgcgaaatacttcttttactcaaacatcatgaactttttcaagactgaaattacgctggccaatggagaaatcaggaagaggccactgatcgaaactaacggagaaacgggcgaaatcgtgtgggacaagggcagggacttcgcaactgttcgcaaagtgctctctatgccgcaagtcaatattgtgaagaaaaccgaagtgcaaaccggcggattttcaaaggaatcgatcctcccaaagagaaatagcgacaagctcattgcacgcaagaaagactgggacccgaagaagtacggaggattcgattcgccgactgtcgcatactccgtcctcgtggtggccaaggtggagaagggaaagagcaaaaagctcaaatccgtcaaagagctgctggggattaccatcatggaacgatcctcgttcgagaagaacccgattgatttcctcgaggcgaagggttacaaggaggtgaagaaggatctgatcatcaaactccccaagtactcactgttcgaactggaaaatggtcggaagcgcatgctggcttcggccggagaactccaaaaaggaaatgagctggccttgcctagcaagtacgtcaacttcctctatcttgcttcgcactacgaaaaactcaaagggtcaccggaagataacgaacagaagcagcttttcgtggagcagcacaagcattatctggatgaaatcatcgaacaaatctccgagttttcaaagcgcgtgatcctcgccgacgccaacctcgacaaagtcctgtcggcctacaataagcatagagataagccgatcagagaacaggccgagaacattatccacttgttcaccctgactaacctgggagccccagccgccttcaagtacttcgatactactatcgatcgcaaaagatacacgtccaccaaggaagttctggacgcgaccctgatccaccaaagcatcactggactctacgaaactaggatcgatctgtcgcagctgggtggcgat

SEQ ID NO: 68 - DNA, 1257 bp, Artificial Sequence
  Synthetic: WT SERPINA1 ORF
atgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataa

SEQ ID NO: 69 - DNA, 1257 bp, Artificial Sequence
  Synthetic: SERPINA1 ORF using codons of Table 5
atgccatcttctgtctcttggggtatcttgttgttggccggtttgtgctgcttggtcccagtctctttggccgaagacccacaaggtgacgccgcccaaaagaccgacacctctcaccacgaccaagaccacccaaccttcaacaagatcaccccaaacttggccgaattcgccttctctttgtacagacaattggcccaccaatctaactctaccaacatcttcttctctccagtctctatcgccaccgccttcgccatgttgtctttgggtaccaaggccgacacccacgacgaaatcttggaaggtttgaacttcaacttgaccgaaatcccagaagcccaaatccacgaaggtttccaagaattgttgagaaccttgaaccaaccagactctcaattgcaattgaccaccggtaacggtttgttcttgtctgaaggtttgaagttggtcgacaagttcttggaagacgtcaagaagttgtaccactctgaagccttcaccgtcaacttcggtgacaccgaagaagccaagaagcaaatcaacgactacgtcgaaaagggtacccaaggtaagatcgtcgacttggtcaaggaattggacagagacaccgtcttcgccttggtcaactacatcttcttcaagggtaagtgggaaagaccattcgaagtcaaggacaccgaagaagaagacttccacgtcgaccaagtcaccaccgtcaaggtcccaatgatgaagagattgggtatgttcaacatccaacactgcaagaagttgtcttcttgggtcttgttgatgaagtacttgggtaacgccaccgccatcttcttcttgccagacgaaggtaagttgcaacacttggaaaacgaattgacccacgacatcatcaccaagttcttggaaaacgaagacagaagatctgcctctttgcacttgccaaagttgtctatcaccggtacctacgacttgaagtctgtcttgggtcaattgggtatcaccaaggtcttctctaacggtgccgacttgtctggtgtcaccgaagaagccccattgaagttgtctaaggccgtccacaaggccgtcttgaccatcgacgaaaagggtaccgaagccgccggtgccatgttcttggaagccatcccaatgtctatcccaccagaagtcaagttcaacaagccattcgtcttcttgatgatcgaacaaaacaccaagtctccattgttcatgggtaaggtcgtcaacccaacccaaaagtaa

SEQ ID NO: 70 - DNA, 1257 bp, Artificial Sequence
  Synthetic: SERPINA1 ORF using codons of Table 6
atgccgtcgtcggtctcgtggggaatcctgctgctggcaggactgtgctgcctggtcccggtctcgctggcagaagacccgcagggagacgcagcacagaagacagacacatcgcaccacgaccaggaccacccgacattcaacaagatcacaccgaacctggcagaattcgcattctcgctgtacagacagctggcacaccagtcgaactcgacaaacatcttcttctcgccggtctcgatcgcaacagcattcgcaatgctgtcgctgggaacaaaggcagacacacacgacgaaatcctggaaggactgaacttcaacctgacagaaatcccggaagcacagatccacgaaggattccaggaactgctgagaacactgaaccagccggactcgcagctgcagctgacaacaggaaacggactgttcctgtcggaaggactgaagctggtcgacaagttcctggaagacgtcaagaagctgtaccactcggaagcattcacagtcaacttcggagacacagaagaagcaaagaagcagatcaacgactacgtcgaaaagggaacacagggaaagatcgtcgacctggtcaaggaactggacagagacacagtcttcgcactggtcaactacatcttcttcaagggaaagtgggaaagaccgttcgaagtcaaggacacagaagaagaagacttccacgtcgaccaggtcacaacagtcaaggtcccgatgatgaagagactgggaatgttcaacatccagcactgcaagaagctgtcgtcgtgggtcctgctgatgaagtacctgggaaacgcaacagcaatcttcttcctgccggacgaaggaaagctgcagcacctggaaaacgaactgacacacgacatcatcacaaagttcctggaaaacgaagacagaagatcggcatcgctgcacctgccgaagctgtcgatcacaggaacatacgacctgaagtcggtcctgggacagctgggaatcacaaaggtcttctcgaacggagcagacctgtcgggagtcacagaagaagcaccgctgaagctgtcgaaggcagtccacaaggcagtcctgacaatcgacgaaaagggaacagaagcagcaggagcaatgttcctggaagcaatcccgatgtcgatcccgccggaagtcaagttcaacaagccgttcgtcttcctgatgatcgaacagaacacaaagtcgccgctgttcatgggaaaggtcgtcaacccgacacagaagtga

SEQ ID NO: 71 - DNA, 1257 bp, Artificial Sequence
  Synthetic: SERPINA1 ORF 1.1
atgcccagcagcgtgagctggggcatcctgctgctggccggcctgtgctgcctggtgcccgtgagcctggccgaggacccccagggcgacgccgcccagaagacggacacgagccaccacgaccaggaccaccccacgttcaacaagatcacgcccaacctggccgagttcgccttcagcctgtaccggcagctggcccaccagagcaacagcacgaacatcttcttcagccccgtgagcatcgccacggccttcgccatgctgagcctgggcacgaaggccgacacgcacgacgagatcctggagggcctgaacttcaacctgacggagatccccgaggcccagatccacgagggcttccaggagctgctgcggacgctgaaccagcccgacagccagctgcagctgacgacgggcaacggcctgttcctgagcgagggcctgaagctggtggacaagttcctggaggacgtgaagaagctgtaccacagcgaggccttcacggtgaacttcggcgacacggaggaggccaagaagcagatcaacgactacgtggagaagggcacgcagggcaagatcgtggacctggtgaaggagctggaccgggacacggtgttcgccctggtgaactacatcttcttcaagggcaagtgggagcggcccttcgaggtgaaggacacggaggaggaggacttccacgtggaccaggtgacgacggtgaaggtgcccatgatgaagcggctgggcatgttcaacatccagcactgcaagaagctgagcagctgggtgctgctgatgaagtacctgggcaacgccacggccatcttcttcctgcccgacgagggcaagctgcagcacctggagaacgagctgacgcacgacatcatcacgaagttcctggagaacgaggaccggcggagcgccagcctgcacctgcccaagctgagcatcacgggcacgtacgacctgaagagcgtgctgggccagctgggcatcacgaaggtgttcagcaacggcgccgacctgagcggcgtgacggaggaggcccccctgaagctgagcaaggccgtgcacaaggccgtgctgacgatcgacgagaagggcacggaggccgccggcgccatgttcctggaggccatccccatgagcatcccccccgaggtgaagttcaacaagcccttcgtgttcctgatgatcgagcagaacacgaagagccccctgttcatgggcaaggtggtgaaccccacgcagaagtag

SEQ ID NO: 72 - DNA, 1257 bp, Artificial Sequence
  Synthetic: SERPINA1 ORF 1.2
atgccctcgtcggtctcgtggggcatcctcctcctcgcgggcctctgctgcctcgtccccgtctcgctcgcggaggacccccagggcgacgcggcgcagaagacggacacgtcgcaccacgaccaggaccaccccacgttcaacaagatcacgcccaacctcgcggagttcgcgttctcgctctaccgccagctcgcgcaccagtcgaactcgacgaacatcttcttctcgcccgtctcgatcgcgacggcgttcgcgatgctctcgctcggcacgaaggcggacacgcacgacgagatcctcgagggcctcaacttcaacctcacggagatccccgaggcgcagatccacgagggcttccaggagctcctccgcacgctcaaccagcccgactcgcagctccagctcacgacgggcaacggcctcttcctctcggagggcctcaagctcgtcgacaagttcctcgaggacgtcaagaagctctaccactcggaggcgttcacggtcaacttcggcgacacggaggaggcgaagaagcagatcaacgactacgtcgagaagggcacgcagggcaagatcgtcgacctcgtcaaggagctcgaccgcgacacggtcttcgcgctcgtcaactacatcttcttcaagggcaagtgggagcgccccttcgaggtcaaggacacggaggaggaggacttccacgtcgaccaggtcacgacggtcaaggtccccatgatgaagcgcctcggcatgttcaacatccagcactgcaagaagctctcgtcgtgggtcctcctcatgaagtacctcggcaacgcgacggcgatcttcttcctccccgacgagggcaagctccagcacctcgagaacgagctcacgcacgacatcatcacgaagttcctcgagaacgaggaccgccgctcggcgtcgctccacctccccaagctctcgatcacgggcacgtacgacctcaagtcggtcctcggccagctcggcatcacgaaggtcttctcgaacggcgcggacctctcgggcgtcacggaggaggcgcccctcaagctctcgaaggcggtccacaaggcggtcctcacgatcgacgagaagggcacggaggcggcgggcgcgatgttcctcgaggcgatccccatgtcgatcccccccgaggtcaagttcaacaagcccttcgtcttcctcatgatcgagcagaacacgaagtcgcccctcttcatgggcaaggtcgtcaaccccacgcagaagtag

SEQ ID NO: 73 - DNA, 1257 bp, Artificial Sequence
  Synthetic: SERPINA1 ORF 1.3
atgccctcatcggtcagctggggcatcctcctcctcgccgggctctgctgcctcgttcccgtcagcctcgcggaggacccccagggcgacgctgcccagaagacggacacgtcgcaccacgaccaggaccaccccaccttcaacaagatcactcccaatctcgcggagttcgcgttctcgctctaccgccagctcgcgcaccagagcaactcgactaacatcttcttctcgcccgtcagcatcgcgacggcgttcgcgatgctcagcctcggcacgaaggcggacacgcacgacgagatcctcgagggcctcaacttcaatctcacagagatcccagaagcccagatccacgagggcttccaggagctgctgcggacgctcaaccagcctgactcgcagctccagctcacgacgggcaatgggctcttcctcagcgagggcctcaagctcgtcgacaagttcctggaggacgtcaagaagctctaccactcggaagccttcacggtcaacttcggcgacacagaggaagccaagaagcagatcaacgactacgtcgagaaggggactcagggcaagatcgtcgacctcgtcaaggagctggaccgagacacggtcttcgcactggtcaactacatcttcttcaaggggaagtgggagcgccccttcgaagtcaaggacacagaggaggaggacttccacgtcgaccaggtgacgacggtcaaggttcccatgatgaagcgcctcggcatgttcaacatccagcactgcaagaagctcagctcgtgggtcctcctcatgaagtacctcggcaacgcgacggcgatcttcttccttcctgacgagggcaagctccagcacctcgagaacgagctgacgcacgacatcatcacgaagttcctggagaacgaggaccgccgatcggcgtcgctccaccttccaaagctcagcatcacgggcacctacgacctcaagtcggtcctcggccagctcggcatcacgaaggtcttctcgaatggtgccgacctcagcggcgtcacagaggaagcccccctcaagctcagcaaggctgtgcacaaggctgtgctcacgatcgacgagaaggggacagaagctgccggtgccatgttcctggaagccatccccatgagcatcccaccagaagtcaagttcaacaagcccttcgtcttcctgatgatagagcagaacacgaagtcgcccctcttcatgggcaaggtcgtcaaccccactcaaaagtag

SEQ ID NO: 74 - protein, 418 aa, Artificial Sequence
  Synthetic: SERPINA1 WT amino acid sequence
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 75 - skipped

SEQ ID NO: 76 - DNA, 1622 bp, Artificial Sequence
  Synthetic: SERPINA1 transcript comprising SEQ 70
gggtcccgcagtcggcgtccagcggctctgcttgttcgtgtgtgtgtcgttgcaggccttattcggatcagccaccatgccgtcgtcggtctcgtggggaatcctgctgctggcaggactgtgctgcctggtcccggtctcgctggcagaagacccgcagggagacgcagcacagaagacagacacatcgcaccacgaccaggaccacccgacattcaacaagatcacaccgaacctggcagaattcgcattctcgctgtacagacagctggcacaccagtcgaactcgacaaacatcttcttctcgccggtctcgatcgcaacagcattcgcaatgctgtcgctgggaacaaaggcagacacacacgacgaaatcctggaaggactgaacttcaacctgacagaaatcccggaagcacagatccacgaaggattccaggaactgctgagaacactgaaccagccggactcgcagctgcagctgacaacaggaaacggactgttcctgtcggaaggactgaagctggtcgacaagttcctggaagacgtcaagaagctgtaccactcggaagcattcacagtcaacttcggagacacagaagaagcaaagaagcagatcaacgactacgtcgaaaagggaacacagggaaagatcgtcgacctggtcaaggaactggacagagacacagtcttcgcactggtcaactacatcttcttcaagggaaagtgggaaagaccgttcgaagtcaaggacacagaagaagaagacttccacgtcgaccaggtcacaacagtcaaggtcccgatgatgaagagactgggaatgttcaacatccagcactgcaagaagctgtcgtcgtgggtcctgctgatgaagtacctgggaaacgcaacagcaatcttcttcctgccggacgaaggaaagctgcagcacctggaaaacgaactgacacacgacatcatcacaaagttcctggaaaacgaagacagaagatcggcatcgctgcacctgccgaagctgtcgatcacaggaacatacgacctgaagtcggtcctgggacagctgggaatcacaaaggtcttctcgaacggagcagacctgtcgggagtcacagaagaagcaccgctgaagctgtcgaaggcagtccacaaggcagtcctgacaatcgacgaaaagggaacagaagcagcaggagcaatgttcctggaagcaatcccgatgtcgatcccgccggaagtcaagttcaacaagccgttcgtcttcctgatgatcgaacagaacacaaagtcgccgctgttcatgggaaaggtcgtcaacccgacacagaagtagctagccatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctcgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 77 - DNA, 1626 bp, Artificial Sequence
  Synthetic: SERPINA1 transcript comprising SEQ 69
gggtcccgcagtcggcgtccagcggctctgcttgttcgtgtgtgtgtcgttgcaggccttattcggatcagccaccatgccatcttctgtctcttggggtatcttgttgttggccggtttgtgctgcttggtcccagtctctttggccgaagacccacaaggtgacgccgcccaaaagaccgacacctctcaccacgaccaagaccacccaaccttcaacaagatcaccccaaacttggccgaattcgccttctctttgtacagacaattggcccaccaatctaactctaccaacatcttcttctctccagtctctatcgccaccgccttcgccatgttgtctttgggtaccaaggccgacacccacgacgaaatcttggaaggtttgaacttcaacttgaccgaaatcccagaagcccaaatccacgaaggtttccaagaattgttgagaaccttgaaccaaccagactctcaattgcaattgaccaccggtaacggtttgttcttgtctgaaggtttgaagttggtcgacaagttcttggaagacgtcaagaagttgtaccactctgaagccttcaccgtcaacttcggtgacaccgaagaagccaagaagcaaatcaacgactacgtcgaaaagggtacccaaggtaagatcgtcgacttggtcaaggaattggacagagacaccgtcttcgccttggtcaactacatcttcttcaagggtaagtgggaaagaccattcgaagtcaaggacaccgaagaagaagacttccacgtcgaccaagtcaccaccgtcaaggtcccaatgatgaagagattgggtatgttcaacatccaacactgcaagaagttgtcttcttgggtcttgttgatgaagtacttgggtaacgccaccgccatcttcttcttgccagacgaaggtaagttgcaacacttggaaaacgaattgacccacgacatcatcaccaagttcttggaaaacgaagacagaagatctgcctctttgcacttgccaaagttgtctatcaccggtacctacgacttgaagtctgtcttgggtcaattgggtatcaccaaggtcttctctaacggtgccgacttgtctggtgtcaccgaagaagccccattgaagttgtctaaggccgtccacaaggccgtcttgaccatcgacgaaaagggtaccgaagccgccggtgccatgttcttggaagccatcccaatgtctatcccaccagaagtcaagttcaacaagccattcgtcttcttgatgatcgaacaaaacaccaagtctccattgttcatgggtaaggtcgtcaacccaacccaaaagtagctagccatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctcgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 78 - DNA, 1640 bp, Artificial Sequence
  Synthetic: SERPINA1 transcript comprising SEQ 71
gggtcccgcagtcggcgtccagcggctctgcttgttcgtgtgtgtgtcgttgcaggccttattccgccaccatgcccagcagcgtgagctggggcatcctgctgctggccggcctgtgctgcctggtgcccgtgagcctggccgaggacccccagggcgacgccgcccagaagacggacacgagccaccacgaccaggaccaccccacgttcaacaagatcacgcccaacctggccgagttcgccttcagcctgtaccggcagctggcccaccagagcaacagcacgaacatcttcttcagccccgtgagcatcgccacggccttcgccatgctgagcctgggcacgaaggccgacacgcacgacgagatcctggagggcctgaacttcaacctgacggagatccccgaggcccagatccacgagggcttccaggagctgctgcggacgctgaaccagcccgacagccagctgcagctgacgacgggcaacggcctgttcctgagcgagggcctgaagctggtggacaagttcctggaggacgtgaagaagctgtaccacagcgaggccttcacggtgaacttcggcgacacggaggaggccaagaagcagatcaacgactacgtggagaagggcacgcagggcaagatcgtggacctggtgaaggagctggaccgggacacggtgttcgccctggtgaactacatcttcttcaagggcaagtgggagcggcccttcgaggtgaaggacacggaggaggaggacttccacgtggaccaggtgacgacggtgaaggtgcccatgatgaagcggctgggcatgttcaacatccagcactgcaagaagctgagcagctgggtgctgctgatgaagtacctgggcaacgccacggccatcttcttcctgcccgacgagggcaagctgcagcacctggagaacgagctgacgcacgacatcatcacgaagttcctggagaacgaggaccggcggagcgccagcctgcacctgcccaagctgagcatcacgggcacgtacgacctgaagagcgtgctgggccagctgggcatcacgaaggtgttcagcaacggcgccgacctgagcggcgtgacggaggaggcccccctgaagctgagcaaggccgtgcacaaggccgtgctgacgatcgacgagaagggcacggaggccgccggcgccatgttcctggaggccatccccatgagcatcccccccgaggtgaagttcaacaagcccttcgtgttcctgatgatcgagcagaacacgaagagccccctgttcatgggcaaggtggtgaaccccacgcagaagtagtagtgaagcttctagccatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctcgagaaaaaaaaaaaaaaaaaaaaaaaaaaaaaagcgaaaaaaaaaaaaaaaaaaaaaaaaaaaaaaccgaaaaaaaaaaaaaaaaaaaaaaaaaaaaaaaaaaaaaaat

SEQ ID NO: 79 - DNA, 1640 bp, Artificial Sequence
  Synthetic: SERPINA1 transcript comprising SEQ 72
gggtcccgcagtcggcgtccagcggctctgcttgttcgtgtgtgtgtcgttgcaggccttattccgccaccatgccctcgtcggtctcgtggggcatcctcctcctcgcgggcctctgctgcctcgtccccgtctcgctcgcggaggacccccagggcgacgcggcgcagaagacggacacgtcgcaccacgaccaggaccaccccacgttcaacaagatcacgcccaacctcgcggagttcgcgttctcgctctaccgccagctcgcgcaccagtcgaactcgacgaacatcttcttctcgcccgtctcgatcgcgacggcgttcgcgatgctctcgctcggcacgaaggcggacacgcacgacgagatcctcgagggcctcaacttcaacctcacggagatccccgaggcgcagatccacgagggcttccaggagctcctccgcacgctcaaccagcccgactcgcagctccagctcacgacgggcaacggcctcttcctctcggagggcctcaagctcgtcgacaagttcctcgaggacgtcaagaagctctaccactcggaggcgttcacggtcaacttcggcgacacggaggaggcgaagaagcagatcaacgactacgtcgagaagggcacgcagggcaagatcgtcgacctcgtcaaggagctcgaccgcgacacggtcttcgcgctcgtcaactacatcttcttcaagggcaagtgggagcgccccttcgaggtcaaggacacggaggaggaggacttccacgtcgaccaggtcacgacggtcaaggtccccatgatgaagcgcctcggcatgttcaacatccagcactgcaagaagctctcgtcgtgggtcctcctcatgaagtacctcggcaacgcgacggcgatcttcttcctccccgacgagggcaagctccagcacctcgagaacgagctcacgcacgacatcatcacgaagttcctcgagaacgaggaccgccgctcggcgtcgctccacctccccaagctctcgatcacgggcacgtacgacctcaagtcggtcctcggccagctcggcatcacgaaggtcttctcgaacggcgcggacctctcgggcgtcacggaggaggcgcccctcaagctctcgaaggcggtccacaaggcggtcctcacgatcgacgagaagggcacggaggcggcgggcgcgatgttcctcgaggcgatccccatgtcgatcccccccgaggtcaagttcaacaagcccttcgtcttcctcatgatcgagcagaacacgaagtcgcccctcttcatgggcaaggtcgtcaaccccacgcagaagtagtagtagagcttctagccatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctcgagaaaaaaaaaaaaaaaaaaaaaaaaaaaaaagcgaaaaaaaaaaaaaaaaaaaaaaaaaaaaaaccgaaaaaaaaaaaaaaaaaaaaaaaaaaaaaaaaaaaaaaat

SEQ ID NO: 80 - DNA, 1640 bp, Artificial Sequence
  Synthetic: SERPINA1 transcript comprising SEQ 73
gggtcccgcagtcggcgtccagcggctctgcttgttcgtgtgtgtgtcgttgcaggccttattccgccaccatgccctcatcggtcagctggggcatcctcctcctcgccgggctctgctgcctcgttcccgtcagcctcgcggaggacccccagggcgacgctgcccagaagacggacacgtcgcaccacgaccaggaccaccccaccttcaacaagatcactcccaatctcgcggagttcgcgttctcgctctaccgccagctcgcgcaccagagcaactcgactaacatcttcttctcgcccgtcagcatcgcgacggcgttcgcgatgctcagcctcggcacgaaggcggacacgcacgacgagatcctcgagggcctcaacttcaatctcacagagatcccagaagcccagatccacgagggcttccaggagctgctgcggacgctcaaccagcctgactcgcagctccagctcacgacgggcaatgggctcttcctcagcgagggcctcaagctcgtcgacaagttcctggaggacgtcaagaagctctaccactcggaagccttcacggtcaacttcggcgacacagaggaagccaagaagcagatcaacgactacgtcgagaaggggactcagggcaagatcgtcgacctcgtcaaggagctggaccgagacacggtcttcgcactggtcaactacatcttcttcaaggggaagtgggagcgccccttcgaagtcaaggacacagaggaggaggacttccacgtcgaccaggtgacgacggtcaaggttcccatgatgaagcgcctcggcatgttcaacatccagcactgcaagaagctcagctcgtgggtcctcctcatgaagtacctcggcaacgcgacggcgatcttcttccttcctgacgagggcaagctccagcacctcgagaacgagctgacgcacgacatcatcacgaagttcctggagaacgaggaccgccgatcggcgtcgctccaccttccaaagctcagcatcacgggcacctacgacctcaagtcggtcctcggccagctcggcatcacgaaggtcttctcgaatggtgccgacctcagcggcgtcacagaggaagcccccctcaagctcagcaaggctgtgcacaaggctgtgctcacgatcgacgagaaggggacagaagctgccggtgccatgttcctggaagccatccccatgagcatcccaccagaagtcaagttcaacaagcccttcgtcttcctgatgatagagcagaacacgaagtcgcccctcttcatgggcaaggtcgtcaaccccactcaaaagtagtgatagagcttctagccatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctcgagaaaaaaaaaaaaaaaaaaaaaaaaaaaaaagcgaaaaaaaaaaaaaaaaaaaaaaaaaaaaaaccgaaaaaaaaaaaaaaaaaaaaaaaaaaaaaaaaaaaaaaat

SEQ ID NO: 81-87 - skipped (7 entries)

SEQ ID NO: 88 - protein, 418 aa, Artificial Sequence
  Synthetic: FAH amino acid sequence
MSFIPVAEDSDFPIHNLPYGVFSTRGDPRPRIGVAIGDQILDLSIIKHLFTGPVLSKHQDVFNQPTLNSFMGLGQAAWKEARVFLQNLLSVSQARLRDDTELRKCAFISQASATMHLPATIGDYTDFYSSRQHATNVGIMFRDKENALMPNWLHLPVGYHGRASSVVVSGTPIRRPMGQMKPDDSKPPVYGACKLLDMELEMAFFVGPGNRLGEPIPISKAHEHIFGMVLMNDWSARDIQKWEYVPLGPFLGKSFGTTVSPWVVPMDALMPFAVPNPKQDPRPLPYLCHDEPYTFDINLSVNLKGEGMSQAATICKSNFKYMYWTMLQQLTHHSVNGCNLRPGDLLASGTISGPEPENFGSMLELSWKGTKPIDLGNGQTRKFLLDGDEVIITGYCQGDGYRIGFGQCAGKVLPALLP

SEQ ID NO: 89 - RNA, 1257 nt, Artificial Sequence
  Synthetic: FAH WT ORF
augucuuuuauuccuguugcugaagauucugauuuuccuauucauaauuuaccuuaugguguuuuuucuacucguggugauccucguccucguauugguguugcuauuggugaucaaauuuuagauuuaucuauuauuaaacauuuauuuacugguccuguuuuaucuaaacaucaagauguuuuuaaucaaccuacuuuaaauucuuuuauggguuuaggucaagcugcuuggaaagaagcucguguuuuuuuacaaaauuuauuaucuguuucucaagcucguuuacgugaugauacugaauuacguaaaugugcuuuuauuucucaagcuucugcuacuaugcauuuaccugcuacuauuggugauuauacugauuuuuauucuucucgucaacaugcuacuaauguugguauuauguuucgugauaaagaaaaugcuuuaaugccuaauugguuacauuuaccuguugguuaucauggucgugcuucuucuguuguuguuucugguacuccuauucgucguccuaugggucaaaugaaaccugaugauucuaaaccuccuguuuauggugcuuguaaauuauuagauauggaauuagaaauggcuuuuuuuguugguccugguaaucguuuaggugaaccuauuccuauuucuaaagcucaugaacauauuuuugguaugguuuuaaugaaugauuggucugcucgugauauucaaaaaugggaauauguuccuuuagguccuuuuuuagguaaaucuuuugguacuacuguuucuccuuggguuguuccuauggaugcuuuaaugccuuuugcuguuccuaauccuaaacaagauccucguccuuuaccuuauuuaugucaugaugaaccuuauacuuuugauauuaauuuaucuguuaauuuaaaaggugaagguaugucucaagcugcuacuauuuguaaaucuaauuuuaaauauauguauuggacuauguuacaacaauuaacucaucauucuguuaaugguuguaauuuacguccuggugauuuauuagcuucugguacuauuucugguccugaaccugaaaauuuugguucuauguuagaauuaucuuggaaagguacuaaaccuauugauuuagguaauggucaaacucguaaauuuuuauuagauggugaugaaguuauuauuacugguuauugucaaggugaugguuaucguauugguuuuggucaaugugcugguaaaguuuuaccugcuuuauuaccuuag

SEQ ID NO: 90 - DNA, 1257 bp, Artificial Sequence
  Synthetic: FAH BP_GCU ORF
atgtcgttcatccccgtcgcggaggactcggacttccccatccacaacctcccctacggcgtcttctcgacgcgcggcgacccccgcccccgcatcggcgtcgcgatcggcgaccagatcctcgacctctcgatcatcaagcacctcttcacgggccccgtcctctcgaagcaccaggacgtcttcaaccagcccacgctcaactcgttcatgggcctcggccaggcggcgtggaaggaggcgcgcgtcttcctccagaacctcctctcggtctcgcaggcgcgcctccgcgacgacacggagctccgcaagtgcgcgttcatctcgcaggcgtcggcgacgatgcacctccccgcgacgatcggcgactacacggacttctactcgtcgcgccagcacgcgacgaacgtcggcatcatgttccgcgacaaggagaacgcgctcatgcccaactggctccacctccccgtcggctaccacggccgcgcgtcgtcggtcgtcgtctcgggcacgcccatccgccgccccatgggccagatgaagcccgacgactcgaagccccccgtctacggcgcgtgcaagctcctcgacatggagctcgagatggcgttcttcgtcggccccggcaaccgcctcggcgagcccatccccatctcgaaggcgcacgagcacatcttcggcatggtcctcatgaacgactggtcggcgcgcgacatccagaagtgggagtacgtccccctcggccccttcctcggcaagtcgttcggcacgacggtctcgccctgggtcgtccccatggacgcgctcatgcccttcgcggtccccaaccccaagcaggacccccgccccctcccctacctctgccacgacgagccctacacgttcgacatcaacctctcggtcaacctcaagggcgagggcatgtcgcaggcggcgacgatctgcaagtcgaacttcaagtacatgtactggacgatgctccagcagctcacgcaccactcggtcaacggctgcaacctccgccccggcgacctcctcgcgtcgggcacgatctcgggccccgagcccgagaacttcggctcgatgctcgagctctcgtggaagggcacgaagcccatcgacctcggcaacggccagacgcgcaagttcctcctcgacggcgacgaggtcatcatcacgggctactgccagggcgacggctaccgcatcggcttcggccagtgcgcgggcaaggtcctccccgcgctcctcccctag

SEQ ID NO: 91 - DNA, 1260 bp, Artificial Sequence
  Synthetic: FAH GP_BP_BS_GCU ORF
atgtcgttcatccccgtcgcggaggactcggacttccccatccacaatcttccatacggcgtcttctcgactcgcggcgacccccgcccccgcatcggcgtcgcgatcggcgaccagatcctcgacctcagcatcatcaagcacctcttcacgggccccgtcctcagcaagcaccaggacgtcttcaaccagcccaccctcaactcgttcatgggcctcggccaggctgcctggaaggaagcccgcgtcttcctgcagaatctcctcagcgtcagccaggcccgcctgcgagacgacacagagctgcggaagtgcgcgttcatctcgcaggcctcggcgacgatgcaccttccagcgacgatcggcgactacacggacttctactcgtcgcgccagcacgcgacgaacgttggcatcatgttccgagacaaggagaacgcactgatgcccaactggctccaccttccagttggctaccacggccgcgcgtcgtcggtcgtcgtcagcggcactcccatccgccgccccatgggccagatgaagcctgacgactcgaagccacccgtctacggtgcctgcaagctcctcgacatggagctggagatggcgttcttcgttggccctggcaaccgcctcggtgagcccatccccatctcgaaggcgcacgagcacatcttcggcatggtcctcatgaacgactggtcggcccgagacatccagaagtgggagtacgttcccctcggccccttcctgggcaagtcgttcggcacgacggtcagcccctgggtcgttcccatggacgcactgatgcccttcgctgttcccaaccccaagcaggacccccgcccccttccatacctctgccacgacgagccctacacgttcgacatcaatctcagcgtcaatctcaagggtgagggcatgtcgcaggctgccacgatctgcaagtcgaacttcaagtacatgtactggacgatgctccagcagctcacgcaccactcggtcaatgggtgcaatctgcggcctggcgacctcctcgcgtcgggcacgatctcgggcccagagccagagaacttcggctcgatgctcgagctcagctggaaggggacgaagcccatcgacctcggcaatgggcagacgcgcaagttcctgctcgacggcgacgaagtcatcatcacgggctactgccagggcgacggctaccgcatcggcttcggccagtgcgccgggaaggtccttccagcactgcttccctcatag

SEQ ID NO: 92 - DNA, 1260 bp, Artificial Sequence
  Synthetic: FAH GS_BS_GCU ORF
atgagcttcatccccgtcgcggaggacagcgacttccccatccacaatcttccatacggcgtcttctcgactcgcggcgacccccgcccccgcatcggcgtcgcgatcggcgaccagatcctcgacctcagcatcatcaagcacctcttcacgggccccgtcctcagcaagcaccaggacgtcttcaaccagcccaccctcaacagcttcatgggcctcggccaggctgcctggaaggaagcccgcgtcttcctgcagaatctcctcagcgtcagccaggcccgcctgcgagacgacacagagctgcggaagtgcgcgttcatcagccaggccagcgcgacgatgcaccttccagcgacgatcggcgactacacggacttctacagcagccgccagcacgcgacgaacgttggcatcatgttccgagacaaggagaacgcactgatgcccaactggctccaccttccagttggctaccacggccgcgcgagcagcgtcgtcgtcagcggcactcccatccgccgccccatgggccagatgaagcctgacgacagcaagccacccgtctacggtgcctgcaagctcctcgacatggagctggagatggcgttcttcgttggccctggcaaccgcctcggtgagcccatccccatcagcaaggcgcacgagcacatcttcggcatggtcctcatgaacgactggagcgcccgagacatccagaagtgggagtacgttcccctcggccccttcctgggcaagagcttcggcacgacggtcagcccctgggtcgttcccatggacgcactgatgcccttcgctgttcccaaccccaagcaggacccccgcccccttccatacctctgccacgacgagccctacacgttcgacatcaatctcagcgtcaatctcaagggtgagggcatgagccaggctgccacgatctgcaagagcaacttcaagtacatgtactggacgatgctccagcagctcacgcaccacagcgtcaatgggtgcaatctgcggcctggcgacctcctcgcgagcggcacgatcagcggcccagagccagagaacttcggcagcatgctcgagctcagctggaaggggacgaagcccatcgacctcggcaatgggcagacgcgcaagttcctgctcgacggcgacgaagtcatcatcacgggctactgccagggcgacggctaccgcatcggcttcggccagtgcgccgggaaggtccttccagcactgcttccctcatag

SEQ ID NO: 93 - DNA, 1260 bp, Artificial Sequence
  Synthetic: FAH GS_GCU ORF
atgagcttcatccccgtggcggaggacagcgacttcccgatccacaatcttccatacggcgtgttctcgactcgcggcgacccgcgcccgcgcatcggcgtggcgatcggcgaccagatcctggacctcagcatcatcaagcacctgttcacgggcccggtgctcagcaagcaccaggacgtgttcaaccagcccaccctgaacagcttcatgggcctgggccaggctgcctggaaggaagcccgcgtgttcctgcagaatctcctcagcgtcagccaggcccgcctgcgagacgacacagagctgcggaagtgcgcgttcatcagccaggccagcgcgacgatgcaccttccagcgacgatcggcgactacacggacttctacagcagccgccagcacgcgacgaacgttggcatcatgttccgagacaaggagaacgcactgatgccgaactggctgcaccttccagttggctaccacggccgcgcgagcagcgtggtggtcagcggcactcccatccgccgcccgatgggccagatgaagcctgacgacagcaagccacccgtgtacggtgcctgcaagctgctggacatggagctggagatggcgttcttcgttggcccgggcaaccgcctgggtgagccgatccccatcagcaaggcgcacgagcacatcttcggcatggtgctgatgaacgactggagcgcccgagacatccagaagtgggagtacgttcccctgggcccgttcctgggcaagagcttcggcacgacggtcagcccgtgggtggttcccatggacgcactgatgccgttcgctgttcccaacccgaagcaggacccgcgcccgcttccatacctgtgccacgacgagccgtacacgttcgacatcaatctcagcgtgaatctcaagggtgagggcatgagccaggctgccacgatctgcaagagcaacttcaagtacatgtactggacgatgctgcagcagctgacgcaccacagcgtgaatgggtgcaatctgcggccgggcgacctgctggcgagcggcacgatcagcggcccagagccagagaacttcggcagcatgctggagctcagctggaaggggacgaagccgatcgacctgggcaatgggcagacgcgcaagttcctgctggacggcgacgaagtcatcatcacgggctactgccagggcgacggctaccgcatcggcttcggccagtgcgccgggaaggtgcttccagcactgcttccctcatag

SEQ ID NO: 94 - protein, 464 aa, Artificial Sequence
  Synthetic: GABRD amino acid sequence
MSEATPLDRNDSENTGGLISRPHPWDQSPSCVQEDRAMNDIGDYVGSNLEISWLPNLDGLIAGYARNFRPGIGGPPVNVALALEVASIDHISEANMEYTMTVFLHQSWRDSRLSYNHTNETLGLDSRFVDKLWLPDTFIVNAKSAWFHDVTVENKLIRLQPDGVILYSIRITSTVACDMDLAKYPMDEQECMLDLESYGYSSEDIVYYWSESQEHIHGLDKLQLAQFTITSYRFTTELMNFKSAGQFPRLSLHFHLRRNRGVYIIQSYMPSVLLVAMSWVSFWISQAAVPARVSLGITTVLTMTTLMVSARSSLPRASAIKALDVYFWICYVFVFAALVEYAFAHFNADYRKKQKAKVKVSRPRAEMDVRNAIVLFSLSAAGVTQELAISRRQRRVPGNLMGSYRSVGVETGETKKEGAARSGGQGGIRARLRPIDADTIDIYARAVFPAAFAAVNVIYWAAYA

SEQ ID NO: 95 - RNA, 1395 nt, Artificial Sequence
  Synthetic: GABRD WT ORF
augucugaagcuacuccuuuagaucguaaugauucugaaaauacuggugguuuaauuucucguccucauccuugggaucaaucuccuucuuguguucaagaagaucgugcuaugaaugauauuggugauuauguugguucuaauuuagaaauuucuugguuaccuaauuuagaugguuuaauugcugguuaugcucguaauuuucguccugguauuggugguccuccuguuaauguugcuuuagcuuuagaaguugcuucuauugaucauauuucugaagcuaauauggaauauacuaugacuguuuuuuuacaucaaucuuggcgugauucucguuuaucuuauaaucauacuaaugaaacuuuagguuuagauucucguuuuguugauaaauuaugguuaccugauacuuuuauuguuaaugcuaaaucugcuugguuucaugauguuacuguugaaaauaaauuaauucguuuacaaccugaugguguuauuuuauauucuauucguauuacuucuacuguugcuugugauauggauuuagcuaaauauccuauggaugaacaagaauguauguuagauuuagaaucuuaugguuauucuucugaagauauuguuuauuauuggucugaaucucaagaacauauucaugguuuagauaaauuacaauuagcucaauuuacuauuacuucuuaucguuuuacuacugaauuaaugaauuuuaaaucugcuggucaauuuccucguuuaucuuuacauuuucauuuacgucguaaucgugguguuuauauuauucaaucuuauaugccuucuguuuuauuaguugcuaugucuuggguuucuuuuuggauuucucaagcugcuguuccugcucguguuucuuuagguauuacuacuguuuuaacuaugacuacuuuaaugguuucugcucguucuucuuuaccucgugcuucugcuauuaaagcuuuagauguuuauuuuuggauuuguuauguuuuuguuuuugcugcuuuaguugaauaugcuuuugcucauuuuaaugcugauuaucguaaaaaacaaaaagcuaaaguuaaaguuucucguccucgugcugaaauggauguucguaaugcuauuguuuuauuuucuuuaucugcugcugguguuacucaagaauuagcuauuucucgucgucaacgucguguuccugguaauuuaauggguucuuaucguucuguugguguugaaacuggugaaacuaaaaaagaaggugcugcucguucugguggucaaggugguauucgugcucguuuacguccuauugaugcugauacuauugauauuuaugcucgugcuguuuuuccugcugcuuuugcugcuguuaauguuauuuauugggcugcuuaugcuuag

SEQ ID NO: 96 - DNA, 1395 bp, Artificial Sequence
  Synthetic: GABRD BP_GCU ORF
atgtcggaggcgacgcccctcgaccgcaacgactcggagaacacgggcggcctcatctcgcgcccccacccctgggaccagtcgccctcgtgcgtccaggaggaccgcgcgatgaacgacatcggcgactacgtcggctcgaacctcgagatctcgtggctccccaacctcgacggcctcatcgcgggctacgcgcgcaacttccgccccggcatcggcggcccccccgtcaacgtcgcgctcgcgctcgaggtcgcgtcgatcgaccacatctcggaggcgaacatggagtacacgatgacggtcttcctccaccagtcgtggcgcgactcgcgcctctcgtacaaccacacgaacgagacgctcggcctcgactcgcgcttcgtcgacaagctctggctccccgacacgttcatcgtcaacgcgaagtcggcgtggttccacgacgtcacggtcgagaacaagctcatccgcctccagcccgacggcgtcatcctctactcgatccgcatcacgtcgacggtcgcgtgcgacatggacctcgcgaagtaccccatggacgagcaggagtgcatgctcgacctcgagtcgtacggctactcgtcggaggacatcgtctactactggtcggagtcgcaggagcacatccacggcctcgacaagctccagctcgcgcagttcacgatcacgtcgtaccgcttcacgacggagctcatgaacttcaagtcggcgggccagttcccccgcctctcgctccacttccacctccgccgcaaccgcggcgtctacatcatccagtcgtacatgccctcggtcctcctcgtcgcgatgtcgtgggtctcgttctggatctcgcaggcggcggtccccgcgcgcgtctcgctcggcatcacgacggtcctcacgatgacgacgctcatggtctcggcgcgctcgtcgctcccccgcgcgtcggcgatcaaggcgctcgacgtctacttctggatctgctacgtcttcgtcttcgcggcgctcgtcgagtacgcgttcgcgcacttcaacgcggactaccgcaagaagcagaaggcgaaggtcaaggtctcgcgcccccgcgcggagatggacgtccgcaacgcgatcgtcctcttctcgctctcggcggcgggcgtcacgcaggagctcgcgatctcgcgccgccagcgccgcgtccccggcaacctcatgggctcgtaccgctcggtcggcgtcgagacgggcgagacgaagaaggagggcgcggcgcgctcgggcggccagggcggcatccgcgcgcgcctccgccccatcgacgcggacacgatcgacatctacgcgcgcgcggtcttccccgcggcgttcgcggcggtcaacgtcatctactgggcggcgtacgcgtag

SEQ ID NO: 97 - DNA, 1395 bp, Artificial Sequence
  Synthetic: GABRD GP_BP_BS_GCU ORF
atgtcggaagccactcccctcgaccgcaacgactcggagaacacgggtggcctcatctcgcgcccccacccctgggaccagagcccctcatgcgtccaggaggaccgcgcgatgaacgacatcggcgactacgttggctcgaatctcgagatctcgtggcttccaaatctcgacggcctcatcgccgggtacgcccgcaacttccgccctggcatcggtggcccacccgtcaacgtcgcactggcactggaagtcgcgagcatcgaccacatctcggaagccaacatggagtacacgatgacggtcttcctgcaccagagctggcgagactcgcgcctcagctacaaccacacgaacgagacgctcggcctcgactcgcgcttcgtcgacaagctctggcttcctgacacgttcatcgtcaacgcgaagtcggcgtggttccacgacgtcacggtcgagaacaagctcatccgcctccagcctgacggcgtcatcctctacagcatccgcatcacgtcgactgtcgcgtgcgacatggacctcgcgaagtaccccatggacgagcaggagtgcatgctcgacctcgagtcgtacggctactcgtcggaggacatcgtctactactggtcggagtcgcaggagcacatccacggcctcgacaagctccagctcgcgcagttcacgatcacgtcgtaccgcttcacgacagagctgatgaacttcaagtcggccgggcagttcccccgcctcagcctccacttccacctgcggcgcaaccgcggcgtctacatcatccagagctacatgccctcagtcctcctcgtcgcgatgtcgtgggtcagcttctggatctcgcaggctgctgttcccgcccgcgtcagcctcggcatcacgacggtcctcacgatgacgacgctcatggtcagcgcccgctcgtcgcttccacgcgcgtcggcgatcaaggcactggacgtctacttctggatctgctacgtcttcgtcttcgctgcactggtcgagtacgcgttcgcgcacttcaacgcggactaccgcaagaagcagaaggcgaaggtcaaggtcagccgcccccgcgcggagatggacgtccgcaacgcgatcgtcctcttctcgctcagcgctgccggggtcactcaggagctggcgatctcgcgccgccagcgccgcgttcccggcaatctcatgggctcgtaccgatcggttggcgtcgagacgggtgagacgaagaaggagggtgctgcccgctcgggtggccagggtggcatccgcgcccgcctgcggcccatcgacgcggacacgatcgacatctacgcccgcgctgtgttccctgctgccttcgctgctgtgaacgtcatctactgggctgcctacgcgtag

SEQ ID NO: 98 - DNA, 1395 bp, Artificial Sequence
  Synthetic: GABRD GS_BS_GCU ORF
atgagcgaagccactcccctcgaccgcaacgacagcgagaacacgggtggcctcatcagccgcccccacccctgggaccagagcccctcatgcgtccaggaggaccgcgcgatgaacgacatcggcgactacgttggcagcaatctcgagatcagctggcttccaaatctcgacggcctcatcgccgggtacgcccgcaacttccgccctggcatcggtggcccacccgtcaacgtcgcactggcactggaagtcgcgagcatcgaccacatcagcgaagccaacatggagtacacgatgacggtcttcctgcaccagagctggcgagacagccgcctcagctacaaccacacgaacgagacgctcggcctcgacagccgcttcgtcgacaagctctggcttcctgacacgttcatcgtcaacgcgaagagcgcgtggttccacgacgtcacggtcgagaacaagctcatccgcctccagcctgacggcgtcatcctctacagcatccgcatcacgtcgactgtcgcgtgcgacatggacctcgcgaagtaccccatggacgagcaggagtgcatgctcgacctcgagagctacggctacagcagcgaggacatcgtctactactggagcgagagccaggagcacatccacggcctcgacaagctccagctcgcgcagttcacgatcacgagctaccgcttcacgacagagctgatgaacttcaagagcgccgggcagttcccccgcctcagcctccacttccacctgcggcgcaaccgcggcgtctacatcatccagagctacatgccctcagtcctcctcgtcgcgatgagctgggtcagcttctggatcagccaggctgctgttcccgcccgcgtcagcctcggcatcacgacggtcctcacgatgacgacgctcatggtcagcgcccgcagcagccttccacgcgcgagcgcgatcaaggcactggacgtctacttctggatctgctacgtcttcgtcttcgctgcactggtcgagtacgcgttcgcgcacttcaacgcggactaccgcaagaagcagaaggcgaaggtcaaggtcagccgcccccgcgcggagatggacgtccgcaacgcgatcgtcctcttcagcctcagcgctgccggggtcactcaggagctggcgatcagccgccgccagcgccgcgttcccggcaatctcatgggcagctacaggagcgttggcgtcgagacgggtgagacgaagaaggagggtgctgcccgcagcggtggccagggtggcatccgcgcccgcctgcggcccatcgacgcggacacgatcgacatctacgcccgcgctgtgttccctgctgccttcgctgctgtgaacgtcatctactgggctgcctacgcgtag

SEQ ID NO: 99 - DNA, 1395 bp, Artificial Sequence
  Synthetic: GABRD GS_GCU ORF
atgagcgaagccactcccctggaccgcaacgacagcgagaacacgggtggcctgatcagccgcccgcacccgtgggaccagagcccctcatgcgtgcaggaggaccgcgcgatgaacgacatcggcgactacgttggcagcaatctcgagatcagctggcttccaaatctcgacggcctgatcgccgggtacgcccgcaacttccgcccgggcatcggtggcccacccgtgaacgtggcactggcactggaagtcgcgagcatcgaccacatcagcgaagccaacatggagtacacgatgacggtgttcctgcaccagagctggcgagacagccgcctcagctacaaccacacgaacgagacgctgggcctggacagccgcttcgtggacaagctgtggcttcctgacacgttcatcgtgaacgcgaagagcgcgtggttccacgacgtgacggtggagaacaagctgatccgcctgcagcctgacggcgtgatcctgtacagcatccgcatcacgtcgactgtggcgtgcgacatggacctggcgaagtacccgatggacgagcaggagtgcatgctggacctggagagctacggctacagcagcgaggacatcgtgtactactggagcgagagccaggagcacatccacggcctggacaagctgcagctggcgcagttcacgatcacgagctaccgcttcacgacagagctgatgaacttcaagagcgccgggcagttcccgcgcctcagcctgcacttccacctgcggcgcaaccgcggcgtgtacatcatccagagctacatgccctcagtgctgctggtggcgatgagctgggtcagcttctggatcagccaggctgctgttcccgcccgcgtcagcctgggcatcacgacggtgctgacgatgacgacgctgatggtcagcgcccgcagcagccttccacgcgcgagcgcgatcaaggcactggacgtgtacttctggatctgctacgtgttcgtgttcgctgcactggtggagtacgcgttcgcgcacttcaacgcggactaccgcaagaagcagaaggcgaaggtgaaggtcagccgcccgcgcgcggagatggacgtgcgcaacgcgatcgtgctgttcagcctcagcgctgccggggtgactcaggagctggcgatcagccgccgccagcgccgcgttcccggcaatctcatgggcagctaccgcagcgttggcgtggagacgggtgagacgaagaaggagggtgctgcccgcagcggtggccagggtggcatccgcgcccgcctgcggccgatcgacgcggacacgatcgacatctacgcccgcgctgtgttcccggctgccttcgctgctgtgaacgtgatctactgggctgcctacgcgtag

SEQ ID NO: 100 - protein, 316 aa, Artificial Sequence
  Synthetic: GAPDH amino acid sequence
MGKVKVGVNGFGRIGRLVTRAAFNSGKVDIVAINDPFIDLNYMAENGKLVINGNPITIFQERDPSKIKWGDAGAEYVVESTGVFTTMEKAGAHLQGGAKRVIISAPSADAPMFVMGVNHEKYDNSLKIISNASCTTNCLAPLAKVIHDNFGIVEGLMTTVHAITATQKTVDGPSGKLWRDGRGALQNIIPASTGAAKAVGKVIPELNGKLTGMAFRVPTANVSVVDLTCRLEKPAKYDDIKKVVKQASEGPLKGILGYTEHQVVSSDFNSDTHSSTFDAGAGIALNDHFVKLISWYDNEFGYSNRVVDLMAHMASK

SEQ ID NO: 101 - RNA, 951 nt, Artificial Sequence
  Synthetic: GAPDH WT ORF
auggguaaaguuaaaguugguguuaaugguuuuggucguauuggucguuuaguuacucgugcugcuuuuaauucugguaaaguugauauuguugcuauuaaugauccuuuuauugauuuaaauuauauggcugaaaaugguaaauuaguuauuaaugguaauccuauuacuauuuuucaagaacgugauccuucuaaaauuaaauggggugaugcuggugcugaauauguuguugaaucuacugguguuuuuacuacuauggaaaaagcuggugcucauuuacaagguggugcuaaacguguuauuauuucugcuccuucugcugaugcuccuauguuuguuauggguguuaaucaugaaaaauaugauaauucuuuaaaaauuauuucuaaugcuucuuguacuacuaauuguuuagcuccuuuagcuaaaguuauucaugauaauuuugguauuguugaagguuuaaugacuacuguucaugcuauuacugcuacucaaaaaacuguugaugguccuucugguaaauuauggcgugauggucguggugcuuuacaaaauauuauuccugcuucuacuggugcugcuaaagcuguugguaaaguuauuccugaauuaaaugguaaauuaacugguauggcuuuucguguuccuacugcuaauguuucuguuguugauuuaacuugucguuuagaaaaaccugcuaaauaugaugauauuaaaaaaguuguuaaacaagcuucugaagguccuuuaaaagguauuuuagguuauacugaacaucaaguuguuucuucugauuuuaauucugauacucauucuucuacuuuugaugcuggugcugguauugcuuuaaaugaucauuuuguuaaauuaauuucuugguaugauaaugaauuugguuauucuaaucguguuguugauuuaauggcucauauggcuucuaaauag

SEQ ID NO: 102 - DNA, 951 bp, Artificial Sequence
  Synthetic: GAPDH BP_GCU ORF
atgggcaaggtcaaggtcggcgtcaacggcttcggccgcatcggccgcctcgtcacgcgcgcggcgttcaactcgggcaaggtcgacatcgtcgcgatcaacgaccccttcatcgacctcaactacatggcggagaacggcaagctcgtcatcaacggcaaccccatcacgatcttccaggagcgcgacccctcgaagatcaagtggggcgacgcgggcgcggagtacgtcgtcgagtcgacgggcgtcttcacgacgatggagaaggcgggcgcgcacctccagggcggcgcgaagcgcgtcatcatctcggcgccctcggcggacgcgcccatgttcgtcatgggcgtcaaccacgagaagtacgacaactcgctcaagatcatctcgaacgcgtcgtgcacgacgaactgcctcgcgcccctcgcgaaggtcatccacgacaacttcggcatcgtcgagggcctcatgacgacggtccacgcgatcacggcgacgcagaagacggtcgacggcccctcgggcaagctctggcgcgacggccgcggcgcgctccagaacatcatccccgcgtcgacgggcgcggcgaaggcggtcggcaaggtcatccccgagctcaacggcaagctcacgggcatggcgttccgcgtccccacggcgaacgtctcggtcgtcgacctcacgtgccgcctcgagaagcccgcgaagtacgacgacatcaagaaggtcgtcaagcaggcgtcggagggccccctcaagggcatcctcggctacacggagcaccaggtcgtctcgtcggacttcaactcggacacgcactcgtcgacgttcgacgcgggcgcgggcatcgcgctcaacgaccacttcgtcaagctcatctcgtggtacgacaacgagttcggctactcgaaccgcgtcgtcgacctcatggcgcacatggcgtcgaagtag

SEQ ID NO: 103 - DNA, 951 bp, Artificial Sequence
  Synthetic: GAPDH GP_BP_BS_GCU ORF
atgggcaaggtcaaggttggcgtcaatgggttcggccgcatcggccgcctcgtcacgcgcgctgccttcaactcgggcaaggtcgacatcgtcgcgatcaacgaccccttcatcgacctcaactacatggcggagaatgggaagctcgtcatcaatgggaaccccatcacgatcttccaggagcgagacccctcaaagatcaagtggggcgacgccggtgccgagtacgtcgtcgagtcgactggcgtcttcacgacgatggagaaggccggtgcccacctccagggtggtgccaagcgcgtcatcatctcggcgccctcagcggacgcgcccatgttcgtcatgggcgtcaaccacgagaagtacgacaactcgctcaagatcatctcgaacgcgtcgtgcacgacgaactgcctcgcgcccctcgcgaaggtcatccacgacaacttcggcatcgtcgagggcctcatgacgacggtccacgcgatcacggcgactcaaaagacggtcgacggcccctcaggcaagctctggcgagacggccgcggtgcactgcagaacatcatccccgcgtcgactggtgctgccaaggctgttggcaaggtcatcccagagctgaatgggaagctcacgggcatggcgttccgcgttcccaccgcgaacgtcagcgtcgtcgacctcacgtgccgcctcgagaagcctgcgaagtacgacgacatcaagaaggtcgtcaagcaggcctcggagggccccctcaaggggatcctcggctacacagagcaccaggtggtcagctcggacttcaactcggacacgcactcgtcgactttcgacgccggtgccgggatcgcactgaacgaccacttcgtcaagctcatctcgtggtacgacaacgagttcggctactcgaaccgcgtcgtcgacctcatggcgcacatggcgtcgaagtag

SEQ ID NO: 104 - DNA, 951 bp, Artificial Sequence
  Synthetic: GAPDH GS_BS_GCU ORF
atgggcaaggtcaaggttggcgtcaatgggttcggccgcatcggccgcctcgtcacgcgcgctgccttcaacagcggcaaggtcgacatcgtcgcgatcaacgaccccttcatcgacctcaactacatggcggagaatgggaagctcgtcatcaatgggaaccccatcacgatcttccaggagcgagacccctcaaagatcaagtggggcgacgccggtgccgagtacgtcgtcgagtcgactggcgtcttcacgacgatggagaaggccggtgcccacctccagggtggtgccaagcgcgtcatcatcagcgcgccctcagcggacgcgcccatgttcgtcatgggcgtcaaccacgagaagtacgacaacagcctcaagatcatcagcaacgcgagctgcacgacgaactgcctcgcgcccctcgcgaaggtcatccacgacaacttcggcatcgtcgagggcctcatgacgacggtccacgcgatcacggcgactcaaaagacggtcgacggcccctcaggcaagctctggcgagacggccgcggtgcactgcagaacatcatccccgcgtcgactggtgctgccaaggctgttggcaaggtcatcccagagctgaatgggaagctcacgggcatggcgttccgcgttcccaccgcgaacgtcagcgtcgtcgacctcacgtgccgcctcgagaagcctgcgaagtacgacgacatcaagaaggtcgtcaagcaggccagcgagggccccctcaaggggatcctcggctacacagagcaccaggtggtcagcagcgacttcaacagcgacacgcacagctcgactttcgacgccggtgccgggatcgcactgaacgaccacttcgtcaagctcatcagctggtacgacaacgagttcggctacagcaaccgcgtcgtcgacctcatggcgcacatggcgagcaagtag

SEQ ID NO: 105 - DNA, 951 bp, Artificial Sequence
  Synthetic: GAPDH GS_GCU ORF
atgggcaaggtgaaggttggcgtgaatgggttcggccgcatcggccgcctggtgacgcgcgctgccttcaacagcggcaaggtggacatcgtggcgatcaacgacccgttcatcgacctgaactacatggcggagaatgggaagctggtgatcaatgggaacccgatcacgatcttccaggagcgagacccctcaaagatcaagtggggcgacgccggtgccgagtacgtggtggagtcgactggcgtgttcacgacgatggagaaggccggtgcccacctgcagggtggtgccaagcgcgtgatcatcagcgcgccctcagcggacgcgccgatgttcgtgatgggcgtgaaccacgagaagtacgacaacagcctgaagatcatcagcaacgcgagctgcacgacgaactgcctggcgccgctggcgaaggtgatccacgacaacttcggcatcgtggagggcctgatgacgacggtgcacgcgatcacggcgactcaaaagacggtggacggcccctcaggcaagctgtggcgagacggccgcggtgcactgcagaacatcatccccgcgtcgactggtgctgccaaggctgttggcaaggtgatcccagagctgaatgggaagctgacgggcatggcgttccgcgttcccaccgcgaacgtcagcgtggtggacctgacgtgccgcctggagaagccggcgaagtacgacgacatcaagaaggtggtgaagcaggccagcgagggcccgctgaaggggatcctgggctacacagagcaccaggtggtcagcagcgacttcaacagcgacacgcacagctcgactttcgacgccggtgccgggatcgcactgaacgaccacttcgtgaagctgatcagctggtacgacaacgagttcggctacagcaaccgcgtggtggacctgatggcgcacatggcgagcaagtag

SEQ ID NO: 106 - protein, 535 aa, Artificial Sequence
  Synthetic: GBA1 amino acid sequence
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRR

SEQ ID NO: 107 - RNA, 1608 nt, Artificial Sequence
  Synthetic: GBA1 WT ORF
auggaauuuucuucuccuucucgugaagaauguccuaaaccuuuaucucguguuucuauuauggcugguucuuuaacugguuuauuauuauuacaagcuguuucuugggcuucuggugcucguccuuguauuccuaaaucuuuugguuauucuucuguuguuuguguuuguaaugcuacuuauugugauucuuuugauccuccuacuuuuccugcuuuagguacuuuuucucguuaugaaucuacucguucuggucgucguauggaauuaucuauggguccuauucaagcuaaucauacugguacugguuuauuauuaacuuuacaaccugaacaaaaauuucaaaaaguuaaagguuuugguggugcuaugacugaugcugcugcuuuaaauauuuuagcuuuaucuccuccugcucaaaauuuauuauuaaaaucuuauuuuucugaagaagguauugguuauaauauuauucguguuccuauggcuucuugugauuuuucuauucguacuuauacuuaugcugauacuccugaugauuuucaauuacauaauuuuucuuuaccugaagaagauacuaaauuaaaaauuccuuuaauucaucgugcuuuacaauuagcucaacguccuguuucuuuauuagcuucuccuuggacuucuccuacuugguuaaaaacuaauggugcuguuaaugguaaagguucuuuaaaaggucaaccuggugauauuuaucaucaaacuugggcucguuauuuuguuaaauuuuuagaugcuuaugcugaacauaaauuacaauuuugggcuguuacugcugaaaaugaaccuucugcugguuuauuaucugguuauccuuuucaauguuuagguuuuacuccugaacaucaacgugauuuuauugcucgugauuuagguccuacuuuagcuaauucuacucaucauaauguucguuuauuaauguuagaugaucaacguuuauuauuaccucauugggcuaaaguuguuuuaacugauccugaagcugcuaaauauguucaugguauugcuguucauugguauuuagauuuuuuagcuccugcuaaagcuacuuuaggugaaacucaucguuuauuuccuaauacuauguuauuugcuucugaagcuuguguugguucuaaauuuugggaacaaucuguucguuuagguucuugggaucgugguaugcaauauucucauucuauuauuacuaauuuauuauaucauguuguugguuggacugauuggaauuuagcuuuaaauccugaaggugguccuaauuggguucguaauuuuguugauucuccuauuauuguugauauuacuaaagauacuuuuuauaaacaaccuauguuuuaucauuuaggucauuuuucuaaauuuauuccugaagguucucaacguguugguuuaguugcuucucaaaaaaaugauuuagaugcuguugcuuuaaugcauccugaugguucugcuguuguuguuguuuuaaaucguucuucuaaagauguuccuuuaacuauuaaagauccugcuguugguuuuuuagaaacuauuucuccugguuauucuauucauacuuauuuauggcgucguuag

SEQ ID NO: 108 - DNA, 1608 bp, Artificial Sequence
  Synthetic: GBA1 BP_GCU ORF
atggagttctcgtcgccctcgcgcgaggagtgccccaagcccctctcgcgcgtctcgatcatggcgggctcgctcacgggcctcctcctcctccaggcggtctcgtgggcgtcgggcgcgcgcccctgcatccccaagtcgttcggctactcgtcggtcgtctgcgtctgcaacgcgacgtactgcgactcgttcgacccccccacgttccccgcgctcggcacgttctcgcgctacgagtcgacgcgctcgggccgccgcatggagctctcgatgggccccatccaggcgaaccacacgggcacgggcctcctcctcacgctccagcccgagcagaagttccagaaggtcaagggcttcggcggcgcgatgacggacgcggcggcgctcaacatcctcgcgctctcgccccccgcgcagaacctcctcctcaagtcgtacttctcggaggagggcatcggctacaacatcatccgcgtccccatggcgtcgtgcgacttctcgatccgcacgtacacgtacgcggacacgcccgacgacttccagctccacaacttctcgctccccgaggaggacacgaagctcaagatccccctcatccaccgcgcgctccagctcgcgcagcgccccgtctcgctcctcgcgtcgccctggacgtcgcccacgtggctcaagacgaacggcgcggtcaacggcaagggctcgctcaagggccagcccggcgacatctaccaccagacgtgggcgcgctacttcgtcaagttcctcgacgcgtacgcggagcacaagctccagttctgggcggtcacggcggagaacgagccctcggcgggcctcctctcgggctaccccttccagtgcctcggcttcacgcccgagcaccagcgcgacttcatcgcgcgcgacctcggccccacgctcgcgaactcgacgcaccacaacgtccgcctcctcatgctcgacgaccagcgcctcctcctcccccactgggcgaaggtcgtcctcacggaccccgaggcggcgaagtacgtccacggcatcgcggtccactggtacctcgacttcctcgcgcccgcgaaggcgacgctcggcgagacgcaccgcctcttccccaacacgatgctcttcgcgtcggaggcgtgcgtcggctcgaagttctgggagcagtcggtccgcctcggctcgtgggaccgcggcatgcagtactcgcactcgatcatcacgaacctcctctaccacgtcgtcggctggacggactggaacctcgcgctcaaccccgagggcggccccaactgggtccgcaacttcgtcgactcgcccatcatcgtcgacatcacgaaggacacgttctacaagcagcccatgttctaccacctcggccacttctcgaagttcatccccgagggctcgcagcgcgtcggcctcgtcgcgtcgcagaagaacgacctcgacgcggtcgcgctcatgcaccccgacggctcggcggtcgtcgtcgtcctcaaccgctcgtcgaaggacgtccccctcacgatcaaggaccccgcggtcggcttcctcgagacgatctcgcccggctactcgatccacacgtacctctggcgccgctag

SEQ ID NO: 109 - DNA, 1608 bp, Artificial Sequence
  Synthetic: GBA1 GP_BP_BS_GCU ORF
atggagttctcgtcgccctcaagggaggagtgccccaagcccctcagccgcgtcagcatcatggccgggtcgctcacgggcctcctcctcctccaggctgtcagctgggcgtcgggtgcccgcccctgcatccccaagtcgttcggctactcgtcggtcgtctgcgtctgcaacgcgacctactgcgactcgttcgacccacccaccttccctgcactgggcacgttctcgcgctacgagtcgactcgatcgggccgccgcatggagctcagcatgggccccatccaggccaaccacacgggcacgggcctcctcctcacgctccagccagagcagaagttccagaaggtcaaggggttcggtggtgccatgacggacgctgctgcactgaacatcctcgcactcagcccacccgcgcagaatctcctcctcaagtcgtacttctcggaggagggcatcggctacaacatcatccgcgttcccatggcgtcgtgcgacttcagcatccgcacctacacctacgcggacactcctgacgacttccagctccacaacttctcgcttccagaggaggacacgaagctcaagatccccctcatccaccgcgcactgcagctcgcgcagcgccccgtcagcctcctcgcgtcgccctggacgtcgcccacctggctcaagacgaatggtgctgtgaatgggaaggggtcgctcaaggggcagcccggcgacatctaccaccagacgtgggcccgctacttcgtcaagttcctggacgcgtacgcggagcacaagctccagttctgggctgtgacggcggagaacgagccctcagccgggctcctcagcggctaccccttccagtgcctcggcttcactccagagcaccagcgagacttcatcgcccgagacctcggccccaccctcgcgaactcgactcaccacaacgtccgcctcctcatgctcgacgaccagcgcctcctccttccacactgggcgaaggtcgtcctcacggacccagaagctgccaagtacgtccacggcatcgctgtgcactggtacctcgacttcctggcgcctgcgaaggcgacgctcggtgagacgcaccgcctcttccccaacacgatgctcttcgcgtcggaagcctgcgttggctcgaagttctgggagcagagcgtccgcctcggctcgtgggaccgcggcatgcagtactcgcacagcatcatcacgaatctcctctaccacgtcgttggctggacggactggaatctcgcactgaacccagagggtggccccaactgggtccgcaacttcgtcgactcgcccatcatcgtcgacatcacgaaggacacgttctacaagcagcccatgttctaccacctcggccacttctcgaagttcatcccagagggctcgcagcgcgttggcctcgtcgcgtcgcagaagaacgacctcgacgctgtggcactgatgcaccctgacggctcggctgtggtcgtcgtcctcaaccgatcgtcgaaggacgttcccctcacgatcaaggaccctgctgttggcttcctggagacgatctcgcctggctacagcatccacacctacctctggcgccgctag

SEQ ID NO: 110 - DNA, 1608 bp, Artificial Sequence
  Synthetic: GBA1 GS_BS_GCU ORF
atggagttcagcagcccctcaagggaggagtgccccaagcccctcagccgcgtcagcatcatggccgggagcctcacgggcctcctcctcctccaggctgtcagctgggcgagcggtgcccgcccctgcatccccaagagcttcggctacagcagcgtcgtctgcgtctgcaacgcgacctactgcgacagcttcgacccacccaccttccctgcactgggcacgttcagccgctacgagtcgactaggagcggccgccgcatggagctcagcatgggccccatccaggccaaccacacgggcacgggcctcctcctcacgctccagccagagcagaagttccagaaggtcaaggggttcggtggtgccatgacggacgctgctgcactgaacatcctcgcactcagcccacccgcgcagaatctcctcctcaagagctacttcagcgaggagggcatcggctacaacatcatccgcgttcccatggcgagctgcgacttcagcatccgcacctacacctacgcggacactcctgacgacttccagctccacaacttcagccttccagaggaggacacgaagctcaagatccccctcatccaccgcgcactgcagctcgcgcagcgccccgtcagcctcctcgcgagcccctggacgagccccacctggctcaagacgaatggtgctgtgaatgggaaggggagcctcaaggggcagcccggcgacatctaccaccagacgtgggcccgctacttcgtcaagttcctggacgcgtacgcggagcacaagctccagttctgggctgtgacggcggagaacgagccctcagccgggctcctcagcggctaccccttccagtgcctcggcttcactccagagcaccagcgagacttcatcgcccgagacctcggccccaccctcgcgaactcgactcaccacaacgtccgcctcctcatgctcgacgaccagcgcctcctccttccacactgggcgaaggtcgtcctcacggacccagaagctgccaagtacgtccacggcatcgctgtgcactggtacctcgacttcctggcgcctgcgaaggcgacgctcggtgagacgcaccgcctcttccccaacacgatgctcttcgcgagcgaagcctgcgttggcagcaagttctgggagcagagcgtccgcctcggcagctgggaccgcggcatgcagtacagccacagcatcatcacgaatctcctctaccacgtcgttggctggacggactggaatctcgcactgaacccagagggtggccccaactgggtccgcaacttcgtcgacagccccatcatcgtcgacatcacgaaggacacgttctacaagcagcccatgttctaccacctcggccacttcagcaagttcatcccagagggcagccagcgcgttggcctcgtcgcgagccagaagaacgacctcgacgctgtggcactgatgcaccctgacggcagcgctgtggtcgtcgtcctcaacaggagcagcaaggacgttcccctcacgatcaaggaccctgctgttggcttcctggagacgatcagccctggctacagcatccacacctacctctggcgccgctag

SEQ ID NO: 111 - DNA, 1608 bp, Artificial Sequence
  Synthetic: GBA1 GS_GCU ORF
atggagttcagcagcccctcaagggaggagtgcccgaagccgctcagccgcgtcagcatcatggccgggagcctgacgggcctgctgctgctgcaggctgtcagctgggcgagcggtgcccgcccgtgcatccccaagagcttcggctacagcagcgtggtgtgcgtgtgcaacgcgacctactgcgacagcttcgacccacccaccttcccggcactgggcacgttcagccgctacgagtcgactcgcagcggccgccgcatggagctcagcatgggcccgatccaggccaaccacacgggcacgggcctgctgctgacgctgcagccagagcagaagttccagaaggtgaaggggttcggtggtgccatgacggacgctgctgcactgaacatcctggcactcagcccacccgcgcagaatctcctgctgaagagctacttcagcgaggagggcatcggctacaacatcatccgcgttcccatggcgagctgcgacttcagcatccgcacctacacctacgcggacactcctgacgacttccagctgcacaacttcagccttccagaggaggacacgaagctgaagatccccctgatccaccgcgcactgcagctggcgcagcgcccggtcagcctgctggcgagcccgtggacgagccccacctggctgaagacgaatggtgctgtgaatgggaaggggagcctgaaggggcagcccggcgacatctaccaccagacgtgggcccgctacttcgtgaagttcctggacgcgtacgcggagcacaagctgcagttctgggctgtgacggcggagaacgagccctcagccgggctgctcagcggctacccgttccagtgcctgggcttcactccagagcaccagcgagacttcatcgcccgagacctgggccccaccctggcgaactcgactcaccacaacgtgcgcctgctgatgctggacgaccagcgcctgctgcttccacactgggcgaaggtggtgctgacggacccagaagctgccaagtacgtgcacggcatcgctgtgcactggtacctggacttcctggcgccggcgaaggcgacgctgggtgagacgcaccgcctgttcccgaacacgatgctgttcgcgagcgaagcctgcgttggcagcaagttctgggagcagagcgtgcgcctgggcagctgggaccgcggcatgcagtacagccacagcatcatcacgaatctcctgtaccacgtggttggctggacggactggaatctcgcactgaacccagagggtggcccgaactgggtgcgcaacttcgtggacagcccgatcatcgtggacatcacgaaggacacgttctacaagcagcccatgttctaccacctgggccacttcagcaagttcatcccagagggcagccagcgcgttggcctggtggcgagccagaagaacgacctggacgctgtggcactgatgcaccctgacggcagcgctgtggtggtggtgctgaaccgcagcagcaaggacgttcccctgacgatcaaggacccggctgttggcttcctggagacgatcagcccgggctacagcatccacacctacctgtggcgccgctag

SEQ ID NO: 112 - protein, 428 aa, Artificial Sequence
  Synthetic: GLA amino acid sequence
MQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDL

SEQ ID NO: 113 - RNA, 1287 nt, Artificial Sequence
  Synthetic: GLA WT ORF
augcaauuacguaauccugaauuacauuuagguugugcuuuagcuuuacguuuuuuagcuuuaguuucuugggauauuccuggugcucgugcuuuagauaaugguuuagcucguacuccuacuauggguugguuacauugggaacguuuuauguguaauuuagauugucaagaagaaccugauucuuguauuucugaaaaauuauuuauggaaauggcugaauuaaugguuucugaagguuggaaagaugcugguuaugaauauuuauguauugaugauuguuggauggcuccucaacgugauucugaaggucguuuacaagcugauccucaacguuuuccucaugguauucgucaauuagcuaauuauguucauucuaaagguuuaaaauuagguauuuaugcugauguugguaauaaaacuugugcugguuuuccugguucuuuugguuauuaugauauugaugcucaaacuuuugcugauugggguguugauuuauuaaaauuugaugguuguuauugugauucuuuagaaaauuuagcugaugguuauaaacauaugucuuuagcuuuaaaucguacuggucguucuauuguuuauucuugugaauggccuuuauauauguggccuuuucaaaaaccuaauuauacugaaauucgucaauauuguaaucauuggcguaauuuugcugauauugaugauucuuggaaaucuauuaaaucuauuuuagauuggacuucuuuuaaucaagaacguauuguugauguugcugguccuggugguuggaaugauccugauauguuaguuauugguaauuuugguuuaucuuggaaucaacaaguuacucaaauggcuuuaugggcuauuauggcugcuccuuuauuuaugucuaaugauuuacgucauauuucuccucaagcuaaagcuuuauuacaagauaaagauguuauugcuauuaaucaagauccuuuagguaaacaagguuaucaauuacgucaaggugauaauuuugaaguuugggaacguccuuuaucugguuuagcuugggcuguugcuaugauuaaucgucaagaaauuggugguccucguucuuauacuauugcuguugcuucuuuagguaaagguguugcuuguaauccugcuuguuuuauuacucaauuauuaccuguuaaacguaaauuagguuuuuaugaauggacuucucguuuacguucucauauuaauccuacugguacuguuuuauuacaauuagaaaauacuaugcaaaugucuuuaaaagauuuauag

SEQ ID NO: 114 - DNA, 1287 bp, Artificial Sequence
  Synthetic: GLA BP_GCU ORF
atgcagctccgcaaccccgagctccacctcggctgcgcgctcgcgctccgcttcctcgcgctcgtctcgtgggacatccccggcgcgcgcgcgctcgacaacggcctcgcgcgcacgcccacgatgggctggctccactgggagcgcttcatgtgcaacctcgactgccaggaggagcccgactcgtgcatctcggagaagctcttcatggagatggcggagctcatggtctcggagggctggaaggacgcgggctacgagtacctctgcatcgacgactgctggatggcgccccagcgcgactcggagggccgcctccaggcggacccccagcgcttcccccacggcatccgccagctcgcgaactacgtccactcgaagggcctcaagctcggcatctacgcggacgtcggcaacaagacgtgcgcgggcttccccggctcgttcggctactacgacatcgacgcgcagacgttcgcggactggggcgtcgacctcctcaagttcgacggctgctactgcgactcgctcgagaacctcgcggacggctacaagcacatgtcgctcgcgctcaaccgcacgggccgctcgatcgtctactcgtgcgagtggcccctctacatgtggcccttccagaagcccaactacacggagatccgccagtactgcaaccactggcgcaacttcgcggacatcgacgactcgtggaagtcgatcaagtcgatcctcgactggacgtcgttcaaccaggagcgcatcgtcgacgtcgcgggccccggcggctggaacgaccccgacatgctcgtcatcggcaacttcggcctctcgtggaaccagcaggtcacgcagatggcgctctgggcgatcatggcggcgcccctcttcatgtcgaacgacctccgccacatctcgccccaggcgaaggcgctcctccaggacaaggacgtcatcgcgatcaaccaggaccccctcggcaagcagggctaccagctccgccagggcgacaacttcgaggtctgggagcgccccctctcgggcctcgcgtgggcggtcgcgatgatcaaccgccaggagatcggcggcccccgctcgtacacgatcgcggtcgcgtcgctcggcaagggcgtcgcgtgcaaccccgcgtgcttcatcacgcagctcctccccgtcaagcgcaagctcggcttctacgagtggacgtcgcgcctccgctcgcacatcaaccccacgggcacggtcctcctccagctcgagaacacgatgcagatgtcgctcaaggacctctag

SEQ ID NO: 115 - DNA, 1287 bp, Artificial Sequence
  Synthetic: GLA GP_BP_BS_GCU ORF
atgcagctgcggaacccagagctgcacctcggctgcgcactggcactgcggttcctggcactggtcagctgggacatccccggtgcccgcgcactggacaatgggctcgcccgcactcccaccatgggctggctccactgggagcgcttcatgtgcaatctcgactgccaggaggagcctgactcgtgcatctcggagaagctcttcatggagatggcggagctgatggtcagcgagggctggaaggacgccgggtacgagtacctctgcatcgacgactgctggatggcgccccagcgagactcggagggccgcctccaggccgacccccagcgcttcccccacggcatccgccagctcgcgaactacgtccactcgaaggggctcaagctcggcatctacgcggacgttggcaacaagacgtgcgccgggttccctggctcgttcggctactacgacatcgacgcgcagacgttcgcggactggggcgtcgacctcctcaagttcgacggctgctactgcgactcgctcgagaatctcgcggacggctacaagcacatgtcgctcgcactgaaccgcacgggccgaagcatcgtctactcgtgcgagtggcccctctacatgtggcccttccagaagcccaactacacagagatccgccagtactgcaaccactggcgcaacttcgcggacatcgacgactcgtggaagagcatcaagagcatcctcgactggacgtcgttcaaccaggagcgcatcgtcgacgtcgccgggcctggtggctggaacgaccctgacatgctcgtcatcggcaacttcggcctcagctggaaccagcaggtgactcaaatggcactgtgggcgatcatggctgcccccctcttcatgtcgaacgacctgcggcacatctcgccccaggccaaggcactgctccaggacaaggacgtcatcgcgatcaaccaggaccccctcggcaagcagggctaccagctgcggcagggcgacaacttcgaagtctgggagcgccccctcagcggcctcgcgtgggctgtggcgatgataaaccgccaggagatcggtggcccccgatcgtacacgatcgctgtggcgtcgctcggcaagggggtcgcgtgcaaccctgcgtgcttcatcactcaactccttccagtcaagcgcaagctcggcttctacgagtggacgtcgcgcctgcggtcgcacatcaaccccaccggcacggtcctcctccagctcgagaacacgatgcagatgtcgctcaaggacctctag

SEQ ID NO: 116 - DNA, 1287 bp, Artificial Sequence
  Synthetic: GLA GS_BS_GCU ORF
atgcagctgcggaacccagagctgcacctcggctgcgcactggcactgcggttcctggcactggtcagctgggacatccccggtgcccgcgcactggacaatgggctcgcccgcactcccaccatgggctggctccactgggagcgcttcatgtgcaatctcgactgccaggaggagcctgacagctgcatcagcgagaagctcttcatggagatggcggagctgatggtcagcgagggctggaaggacgccgggtacgagtacctctgcatcgacgactgctggatggcgccccagcgagacagcgagggccgcctccaggccgacccccagcgcttcccccacggcatccgccagctcgcgaactacgtccacagcaaggggctcaagctcggcatctacgcggacgttggcaacaagacgtgcgccgggttccctggcagcttcggctactacgacatcgacgcgcagacgttcgcggactggggcgtcgacctcctcaagttcgacggctgctactgcgacagcctcgagaatctcgcggacggctacaagcacatgagcctcgcactgaaccgcacgggcaggagcatcgtctacagctgcgagtggcccctctacatgtggcccttccagaagcccaactacacagagatccgccagtactgcaaccactggcgcaacttcgcggacatcgacgacagctggaagagcatcaagagcatcctcgactggacgagcttcaaccaggagcgcatcgtcgacgtcgccgggcctggtggctggaacgaccctgacatgctcgtcatcggcaacttcggcctcagctggaaccagcaggtgactcaaatggcactgtgggcgatcatggctgcccccctcttcatgagcaacgacctgcggcacatcagcccccaggccaaggcactgctccaggacaaggacgtcatcgcgatcaaccaggaccccctcggcaagcagggctaccagctgcggcagggcgacaacttcgaagtctgggagcgccccctcagcggcctcgcgtgggctgtggcgatgataaaccgccaggagatcggtggccccaggagctacacgatcgctgtggcgagcctcggcaagggggtcgcgtgcaaccctgcgtgcttcatcactcaactccttccagtcaagcgcaagctcggcttctacgagtggacgagccgcctgcggagccacatcaaccccaccggcacggtcctcctccagctcgagaacacgatgcagatgagcctcaaggacctctag

SEQ ID NO: 117 - DNA, 1287 bp, Artificial Sequence
  Synthetic: GLA GS_GCU ORF
atgcagctgcggaacccagagctgcacctgggctgcgcactggcactgcggttcctggcactggtcagctgggacatccccggtgcccgcgcactggacaatgggctggcccgcactcccaccatgggctggctgcactgggagcgcttcatgtgcaatctcgactgccaggaggagcctgacagctgcatcagcgagaagctgttcatggagatggcggagctgatggtcagcgagggctggaaggacgccgggtacgagtacctgtgcatcgacgactgctggatggcgccgcagcgagacagcgagggccgcctgcaggccgacccgcagcgcttcccgcacggcatccgccagctggcgaactacgtgcacagcaaggggctgaagctgggcatctacgcggacgttggcaacaagacgtgcgccgggttcccgggcagcttcggctactacgacatcgacgcgcagacgttcgcggactggggcgtggacctgctgaagttcgacggctgctactgcgacagcctggagaatctcgcggacggctacaagcacatgagcctggcactgaaccgcacgggccgcagcatcgtgtacagctgcgagtggccgctgtacatgtggccgttccagaagccgaactacacagagatccgccagtactgcaaccactggcgcaacttcgcggacatcgacgacagctggaagagcatcaagagcatcctggactggacgagcttcaaccaggagcgcatcgtggacgtggccgggccgggtggctggaacgaccctgacatgctggtgatcggcaacttcggcctcagctggaaccagcaggtgactcaaatggcactgtgggcgatcatggctgccccgctgttcatgagcaacgacctgcggcacatcagcccgcaggccaaggcactgctgcaggacaaggacgtgatcgcgatcaaccaggacccgctgggcaagcagggctaccagctgcggcagggcgacaacttcgaagtctgggagcgcccgctcagcggcctggcgtgggctgtggcgatgataaaccgccaggagatcggtggcccgcgcagctacacgatcgctgtggcgagcctgggcaagggggtggcgtgcaacccggcgtgcttcatcactcaactgcttccagtgaagcgcaagctgggcttctacgagtggacgagccgcctgcggagccacatcaaccccaccggcacggtgctgctgcagctggagaacacgatgcagatgagcctgaaggacctgtag

SEQ ID NO: 118 - protein, 353 aa, Artificial Sequence
  Synthetic: OTC amino acid sequence
MLFNLRILLNNAAFRNGHNFMVRNFRCGQPLQNKVQLKGRDLLTLKNFTGEEIKYMLWLSADLKFRIKQKGEYLPLLQGKSLGMIFEKRSTRTRLSTETGFALLGGHPCFLTTQDIHLGVNESLTDTARVLSSMADAVLARVYKQSDLDTLAKEASIPIINGLSDLYHPIQILADYLTLQEHYSSLKGLTLSWIGDGNNILHSIMMSAAKFGMHLQAATPKGYEPDASVTKLAEQYAKENGTKLLLTNDPLEAAHGGNVLITDTWISMGQEEEKKKRLQAFQGYQVTMKTAKVAASDWTFLHCLPRKPEEVDDEVFYSPRSLVFPEAENRKWTIMAVMVSLLTDYSPQLQKPK

SEQ ID NO: 119 - RNA, 1062 nt, Artificial Sequence
  Synthetic: OTC WT ORF
auguuauuuaauuuacguauuuuauuaaauaaugcugcuuuucguaauggucauaauuuuaugguucguaauuuucguuguggucaaccuuuacaaaauaaaguucaauuaaaaggucgugauuuauuaacuuuaaaaaauuuuacuggugaagaaauuaaauauauguuaugguuaucugcugauuuaaaauuucguauuaaacaaaaaggugaauauuuaccuuuauuacaagguaaaucuuuagguaugauuuuugaaaaacguucuacucguacucguuuaucuacugaaacugguuuugcuuuauuagguggucauccuuguuuuuuaacuacucaagauauucauuuagguguuaaugaaucuuuaacugauacugcucguguuuuaucuucuauggcugaugcuguuuuagcucguguuuauaaacaaucugauuuagauacuuuagcuaaagaagcuucuauuccuauuauuaaugguuuaucugauuuauaucauccuauucaaauuuuagcugauuauuuaacuuuacaagaacauuauucuucuuuaaaagguuuaacuuuaucuuggauuggugaugguaauaauauuuuacauucuauuaugaugucugcugcuaaauuugguaugcauuuacaagcugcuacuccuaaagguuaugaaccugaugcuucuguuacuaaauuagcugaacaauaugcuaaagaaaaugguacuaaauuauuauuaacuaaugauccuuuagaagcugcucauggugguaauguuuuaauuacugauacuuggauuucuaugggucaagaagaagaaaaaaaaaaacguuuacaagcuuuucaagguuaucaaguuacuaugaaaacugcuaaaguugcugcuucugauuggacuuuuuuacauuguuuaccucguaaaccugaagaaguugaugaugaaguuuuuuauucuccucguucuuuaguuuuuccugaagcugaaaaucguaaauggacuauuauggcuguuaugguuucuuuauuaacugauuauucuccucaauuacaaaaaccuaaauag

SEQ ID NO: 120 - DNA, 1062 bp, Artificial Sequence
  Synthetic: OTC BP_GCU ORF
atgctcttcaacctccgcatcctcctcaacaacgcggcgttccgcaacggccacaacttcatggtccgcaacttccgctgcggccagcccctccagaacaaggtccagctcaagggccgcgacctcctcacgctcaagaacttcacgggcgaggagatcaagtacatgctctggctctcggcggacctcaagttccgcatcaagcagaagggcgagtacctccccctcctccagggcaagtcgctcggcatgatcttcgagaagcgctcgacgcgcacgcgcctctcgacggagacgggcttcgcgctcctcggcggccacccctgcttcctcacgacgcaggacatccacctcggcgtcaacgagtcgctcacggacacggcgcgcgtcctctcgtcgatggcggacgcggtcctcgcgcgcgtctacaagcagtcggacctcgacacgctcgcgaaggaggcgtcgatccccatcatcaacggcctctcggacctctaccaccccatccagatcctcgcggactacctcacgctccaggagcactactcgtcgctcaagggcctcacgctctcgtggatcggcgacggcaacaacatcctccactcgatcatgatgtcggcggcgaagttcggcatgcacctccaggcggcgacgcccaagggctacgagcccgacgcgtcggtcacgaagctcgcggagcagtacgcgaaggagaacggcacgaagctcctcctcacgaacgaccccctcgaggcggcgcacggcggcaacgtcctcatcacggacacgtggatctcgatgggccaggaggaggagaagaagaagcgcctccaggcgttccagggctaccaggtcacgatgaagacggcgaaggtcgcggcgtcggactggacgttcctccactgcctcccccgcaagcccgaggaggtcgacgacgaggtcttctactcgccccgctcgctcgtcttccccgaggcggagaaccgcaagtggacgatcatggcggtcatggtctcgctcctcacggactactcgccccagctccagaagcccaagtag

SEQ ID NO: 121 - DNA, 1062 bp, Artificial Sequence
  Synthetic: OTC GP_BP_BS_GCU ORF
atgctcttcaatctgcggatcctcctcaacaacgctgccttccgcaatgggcacaacttcatggtccgcaacttccgctgtgggcagcccctccagaacaaggtccagctcaaggggcgagacctcctcacgctcaagaacttcacgggtgaggagatcaagtacatgctctggctcagcgcggacctcaagttccgcatcaagcagaagggtgagtaccttccactcctccagggcaagtcgctcggcatgatattcgagaagcgatcgactcgcacgcgcctctcgacagagacgggcttcgcactgctcggtggccacccctgcttcctgacgactcaagacatccacctcggcgtcaacgagtcgctcacggacacggcccgcgtcctcagctcgatggcggacgctgtgctcgcccgcgtctacaagcagagcgacctcgacacgctcgcgaaggaagccagcatccccatcatcaatgggctcagcgacctctaccaccccatccagatcctcgcggactacctcacgctccaggagcactactcgtcgctcaaggggctcacgctcagctggatcggcgacggcaacaacatcctccacagcatcatgatgtcggctgccaagttcggcatgcacctccaggctgccactcccaaggggtacgagcctgacgcgtcggtcacgaagctcgcggagcagtacgcgaaggagaatgggacgaagctcctcctcacgaacgaccccctcgaagctgcccacggtggcaacgtcctcatcacggacacgtggatctcgatgggccaggaggaggagaagaagaagcgcctccaggccttccagggctaccaggtgacgatgaagacggcgaaggtcgctgcctcggactggacgttcctgcactgccttccacgcaagccagaggaagtcgacgacgaagtcttctactcgccccgatcgctcgtcttcccagaagccgagaaccgcaagtggacgatcatggctgtgatggtcagcctcctcacggactactcgccccagctccagaagcccaagtag

SEQ ID NO: 122 - DNA, 1062 bp, Artificial Sequence
  Synthetic: OTC GS_BS_GCU ORF
atgctcttcaatctgcggatcctcctcaacaacgctgccttccgcaatgggcacaacttcatggtccgcaacttccgctgtgggcagcccctccagaacaaggtccagctcaaggggcgagacctcctcacgctcaagaacttcacgggtgaggagatcaagtacatgctctggctcagcgcggacctcaagttccgcatcaagcagaagggtgagtaccttccactcctccagggcaagagcctcggcatgatattcgagaagaggtcgactcgcacgcgcctctcgacagagacgggcttcgcactgctcggtggccacccctgcttcctgacgactcaagacatccacctcggcgtcaacgagagcctcacggacacggcccgcgtcctcagcagcatggcggacgctgtgctcgcccgcgtctacaagcagagcgacctcgacacgctcgcgaaggaagccagcatccccatcatcaatgggctcagcgacctctaccaccccatccagatcctcgcggactacctcacgctccaggagcactacagcagcctcaaggggctcacgctcagctggatcggcgacggcaacaacatcctccacagcatcatgatgagcgctgccaagttcggcatgcacctccaggctgccactcccaaggggtacgagcctgacgcgagcgtcacgaagctcgcggagcagtacgcgaaggagaatgggacgaagctcctcctcacgaacgaccccctcgaagctgcccacggtggcaacgtcctcatcacggacacgtggatcagcatgggccaggaggaggagaagaagaagcgcctccaggccttccagggctaccaggtgacgatgaagacggcgaaggtcgctgccagcgactggacgttcctgcactgccttccacgcaagccagaggaagtcgacgacgaagtcttctacagccccaggagcctcgtcttcccagaagccgagaaccgcaagtggacgatcatggctgtgatggtcagcctcctcacggactacagcccccagctccagaagcccaagtag

SEQ ID NO: 123 - DNA, 1062 bp, Artificial Sequence
  Synthetic: OTC GS_GCU ORF
atgctgttcaatctgcggatcctgctgaacaacgctgccttccgcaatgggcacaacttcatggtgcgcaacttccgctgtgggcagcccctgcagaacaaggtgcagctgaaggggcgagacctgctgacgctgaagaacttcacgggtgaggagatcaagtacatgctgtggctcagcgcggacctgaagttccgcatcaagcagaagggtgagtaccttccactgctgcagggcaagagcctgggcatgatattcgagaagcgctcgactcgcacgcgcctctcgacagagacgggcttcgcactgctgggtggccacccgtgcttcctgacgactcaagacatccacctgggcgtgaacgagagcctgacggacacggcccgcgtgctcagcagcatggcggacgctgtgctggcccgcgtgtacaagcagagcgacctggacacgctggcgaaggaagccagcatccccatcatcaatgggctcagcgacctgtaccacccgatccagatcctggcggactacctgacgctgcaggagcactacagcagcctgaaggggctgacgctcagctggatcggcgacggcaacaacatcctgcacagcatcatgatgagcgctgccaagttcggcatgcacctgcaggctgccactcccaaggggtacgagcctgacgcgagcgtgacgaagctggcggagcagtacgcgaaggagaatgggacgaagctgctgctgacgaacgacccgctggaagctgcccacggtggcaacgtgctgatcacggacacgtggatcagcatgggccaggaggaggagaagaagaagcgcctgcaggccttccagggctaccaggtgacgatgaagacggcgaaggtggctgccagcgactggacgttcctgcactgccttccacgcaagccagaggaagtcgacgacgaagtcttctacagcccgcgcagcctggtgttcccagaagccgagaaccgcaagtggacgatcatggctgtgatggtcagcctgctgacggactacagcccgcagctgcagaagccgaagtag

SEQ ID NO: 124 - protein, 451 aa, Artificial Sequence
  Synthetic: PAH amino acid sequence
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKI

SEQ ID NO: 125 - RNA, 1356 nt, Artificial Sequence
  Synthetic: PAH WT ORF
augucuacugcuguuuuagaaaauccugguuuaggucguaaauuaucugauuuuggucaagaaacuucuuauauugaagauaauuguaaucaaaauggugcuauuucuuuaauuuuuucuuuaaaagaagaaguuggugcuuuagcuaaaguuuuacguuuauuugaagaaaaugauguuaauuuaacucauauugaaucucguccuucucguuuaaaaaaagaugaauaugaauuuuuuacucauuuagauaaacguucuuuaccugcuuuaacuaauauuauuaaaauuuuacgucaugauauuggugcuacuguucaugaauuaucucgugauaaaaaaaaagauacuguuccuugguuuccucguacuauucaagaauuagaucguuuugcuaaucaaauuuuaucuuauggugcugaauuagaugcugaucauccugguuuuaaagauccuguuuaucgugcucgucguaaacaauuugcugauauugcuuauaauuaucgucauggucaaccuauuccucguguugaauauauggaagaagaaaaaaaaacuugggguacuguuuuuaaaacuuuaaaaucuuuauauaaaacucaugcuuguuaugaauauaaucauauuuuuccuuuauuagaaaaauauugugguuuucaugaagauaauauuccucaauuagaagauguuucucaauuuuuacaaacuuguacugguuuucguuuacguccuguugcugguuuauuaucuucucgugauuuuuuaggugguuuagcuuuucguguuuuucauuguacucaauauauucgucaugguucuaaaccuauguauacuccugaaccugauauuugucaugaauuauuaggucauguuccuuuauuuucugaucguucuuuugcucaauuuucucaagaaauugguuuagcuucuuuaggugcuccugaugaauauauugaaaaauuagcuacuauuuauugguuuacuguugaauuugguuuauguaaacaaggugauucuauuaaagcuuauggugcugguuuauuaucuucuuuuggugaauuacaauauuguuuaucugaaaaaccuaaauuauuaccuuuagaauuagaaaaaacugcuauucaaaauuauacuguuacugaauuucaaccuuuauauuauguugcugaaucuuuuaaugaugcuaaagaaaaaguucguaauuuugcugcuacuauuccucguccuuuuucuguucguuaugauccuuauacucaacguauugaaguuuuagauaauacucaacaauuaaaaauuuuagcugauucuauuaauucugaaauugguauuuuauguucugcuuuacaaaaaauuuag

SEQ ID NO: 126 - DNA, 1356 bp, Artificial Sequence
  Synthetic: PAH BP_GCU ORF
atgtcgacggcggtcctcgagaaccccggcctcggccgcaagctctcggacttcggccaggagacgtcgtacatcgaggacaactgcaaccagaacggcgcgatctcgctcatcttctcgctcaaggaggaggtcggcgcgctcgcgaaggtcctccgcctcttcgaggagaacgacgtcaacctcacgcacatcgagtcgcgcccctcgcgcctcaagaaggacgagtacgagttcttcacgcacctcgacaagcgctcgctccccgcgctcacgaacatcatcaagatcctccgccacgacatcggcgcgacggtccacgagctctcgcgcgacaagaagaaggacacggtcccctggttcccccgcacgatccaggagctcgaccgcttcgcgaaccagatcctctcgtacggcgcggagctcgacgcggaccaccccggcttcaaggaccccgtctaccgcgcgcgccgcaagcagttcgcggacatcgcgtacaactaccgccacggccagcccatcccccgcgtcgagtacatggaggaggagaagaagacgtggggcacggtcttcaagacgctcaagtcgctctacaagacgcacgcgtgctacgagtacaaccacatcttccccctcctcgagaagtactgcggcttccacgaggacaacatcccccagctcgaggacgtctcgcagttcctccagacgtgcacgggcttccgcctccgccccgtcgcgggcctcctctcgtcgcgcgacttcctcggcggcctcgcgttccgcgtcttccactgcacgcagtacatccgccacggctcgaagcccatgtacacgcccgagcccgacatctgccacgagctcctcggccacgtccccctcttctcggaccgctcgttcgcgcagttctcgcaggagatcggcctcgcgtcgctcggcgcgcccgacgagtacatcgagaagctcgcgacgatctactggttcacggtcgagttcggcctctgcaagcagggcgactcgatcaaggcgtacggcgcgggcctcctctcgtcgttcggcgagctccagtactgcctctcggagaagcccaagctcctccccctcgagctcgagaagacggcgatccagaactacacggtcacggagttccagcccctctactacgtcgcggagtcgttcaacgacgcgaaggagaaggtccgcaacttcgcggcgacgatcccccgccccttctcggtccgctacgacccctacacgcagcgcatcgaggtcctcgacaacacgcagcagctcaagatcctcgcggactcgatcaactcggagatcggcatcctctgctcggcgctccagaagatctag

SEQ ID NO: 127 - DNA, 1356 bp, Artificial Sequence
  Synthetic: PAH GP_BP_BS_GCU ORF
atgtcgactgctgtgctcgagaaccctggcctcggccgcaagctcagcgacttcggccaggagacgtcgtacatcgaggacaactgcaaccagaatggtgccatctcgctcatcttctcgctcaaggaggaagttggtgcactggcgaaggtcctgcggctcttcgaggagaacgacgtcaatctcacgcacatcgagtcgcgcccctcacgcctcaagaaggacgagtacgagttcttcacgcacctcgacaagcgatcgcttccagcactgacgaacatcatcaagatcctgcggcacgacatcggtgccacggtccacgagctcagccgagacaagaagaaggacacggttccctggttcccccgcacgatccaggagctggaccgcttcgcgaaccagatcctcagctacggtgccgagctggacgcggaccaccctggcttcaaggaccccgtctaccgcgcccgccgcaagcagttcgcggacatcgcgtacaactaccgccacggccagcccatcccccgcgtcgagtacatggaggaggagaagaagacgtggggcacggtcttcaagacgctcaagtcgctctacaagacgcacgcgtgctacgagtacaaccacatcttccccctcctcgagaagtactgtgggttccacgaggacaacatcccccagctcgaggacgtcagccagttcctgcagacgtgcacgggcttccgcctgcggcccgtcgccgggctcctcagctcgcgagacttcctgggtggcctcgcgttccgcgtcttccactgcactcaatacatccgccacggctcgaagcccatgtacactccagagcctgacatctgccacgagctgctcggccacgttcccctcttctcggaccgatcgttcgcgcagttctcgcaggagatcggcctcgcgtcgctcggtgcccctgacgagtacatcgagaagctcgcgacgatctactggttcacggtcgagttcggcctctgcaagcagggcgacagcatcaaggcgtacggtgccgggctcctcagctcgttcggtgagctgcagtactgcctcagcgagaagcccaagctccttccactcgagctggagaagacggcgatccagaactacacggtcacagagttccagcccctctactacgtcgcggagtcgttcaacgacgcgaaggagaaggtccgcaacttcgctgccacgatcccccgccccttctcggtccgctacgacccctacactcaacgcatcgaagtcctcgacaacactcaacagctcaagatcctcgcggacagcatcaactcggagatcggcatcctctgctcggcactgcagaagatctag

SEQ ID NO: 128 - DNA, 1356 bp, Artificial Sequence
  Synthetic: PAH GS_BS_GCU ORF
atgtcgactgctgtgctcgagaaccctggcctcggccgcaagctcagcgacttcggccaggagacgagctacatcgaggacaactgcaaccagaatggtgccatcagcctcatcttcagcctcaaggaggaagttggtgcactggcgaaggtcctgcggctcttcgaggagaacgacgtcaatctcacgcacatcgagagccgcccctcacgcctcaagaaggacgagtacgagttcttcacgcacctcgacaagaggagccttccagcactgacgaacatcatcaagatcctgcggcacgacatcggtgccacggtccacgagctcagccgagacaagaagaaggacacggttccctggttcccccgcacgatccaggagctggaccgcttcgcgaaccagatcctcagctacggtgccgagctggacgcggaccaccctggcttcaaggaccccgtctaccgcgcccgccgcaagcagttcgcggacatcgcgtacaactaccgccacggccagcccatcccccgcgtcgagtacatggaggaggagaagaagacgtggggcacggtcttcaagacgctcaagagcctctacaagacgcacgcgtgctacgagtacaaccacatcttccccctcctcgagaagtactgtgggttccacgaggacaacatcccccagctcgaggacgtcagccagttcctgcagacgtgcacgggcttccgcctgcggcccgtcgccgggctcctcagcagccgagacttcctgggtggcctcgcgttccgcgtcttccactgcactcaatacatccgccacggcagcaagcccatgtacactccagagcctgacatctgccacgagctgctcggccacgttcccctcttcagcgacaggagcttcgcgcagttcagccaggagatcggcctcgcgagcctcggtgcccctgacgagtacatcgagaagctcgcgacgatctactggttcacggtcgagttcggcctctgcaagcagggcgacagcatcaaggcgtacggtgccgggctcctcagcagcttcggtgagctgcagtactgcctcagcgagaagcccaagctccttccactcgagctggagaagacggcgatccagaactacacggtcacagagttccagcccctctactacgtcgcggagagcttcaacgacgcgaaggagaaggtccgcaacttcgctgccacgatcccccgccccttcagcgtccgctacgacccctacactcaacgcatcgaagtcctcgacaacactcaacagctcaagatcctcgcggacagcatcaacagcgagatcggcatcctctgcagcgcactgcagaagatctag

SEQ ID NO: 129 - DNA, 1356 bp, Artificial Sequence
  Synthetic: PAH GS_GCU ORF
atgtcgactgctgtgctggagaacccgggcctgggccgcaagctcagcgacttcggccaggagacgagctacatcgaggacaactgcaaccagaatggtgccatcagcctgatcttcagcctgaaggaggaagttggtgcactggcgaaggtgctgcggctgttcgaggagaacgacgtgaatctcacgcacatcgagagccgcccctcacgcctgaagaaggacgagtacgagttcttcacgcacctggacaagcgcagccttccagcactgacgaacatcatcaagatcctgcggcacgacatcggtgccacggtgcacgagctcagccgagacaagaagaaggacacggttccctggttcccgcgcacgatccaggagctggaccgcttcgcgaaccagatcctcagctacggtgccgagctggacgcggaccacccgggcttcaaggacccggtgtaccgcgcccgccgcaagcagttcgcggacatcgcgtacaactaccgccacggccagcccatcccccgcgtggagtacatggaggaggagaagaagacgtggggcacggtgttcaagacgctgaagagcctgtacaagacgcacgcgtgctacgagtacaaccacatcttcccgctgctggagaagtactgtgggttccacgaggacaacatcccccagctggaggacgtcagccagttcctgcagacgtgcacgggcttccgcctgcggccggtggccgggctgctcagcagccgagacttcctgggtggcctggcgttccgcgtgttccactgcactcaatacatccgccacggcagcaagccgatgtacactccagagcctgacatctgccacgagctgctgggccacgttcccctgttcagcgaccgcagcttcgcgcagttcagccaggagatcggcctggcgagcctgggtgcccctgacgagtacatcgagaagctggcgacgatctactggttcacggtggagttcggcctgtgcaagcagggcgacagcatcaaggcgtacggtgccgggctgctcagcagcttcggtgagctgcagtactgcctcagcgagaagccgaagctgcttccactggagctggagaagacggcgatccagaactacacggtgacagagttccagcccctgtactacgtggcggagagcttcaacgacgcgaaggagaaggtgcgcaacttcgctgccacgatcccccgcccgttcagcgtgcgctacgacccgtacactcaacgcatcgaagtcctggacaacactcaacagctgaagatcctggcggacagcatcaacagcgagatcggcatcctgtgcagcgcactgcagaagatctag

SEQ ID NO: 130 - protein, 146 aa, Artificial Sequence
  Synthetic: TTR amino acid sequence
MASHRLLLLCLAGLVFVSEAGPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPK

SEQ ID NO: 131 - RNA, 441 nt, Artificial Sequence
  Synthetic: TTR WT ORF
auggcuucucaucguuuauuauuauuauguuuagcugguuuaguuuuuguuucugaagcugguccuacugguacuggugaaucuaaauguccuuuaaugguuaaaguuuuagaugcuguucgugguucuccugcuauuaauguugcuguucauguuuuucguaaagcugcugaugauacuugggaaccuuuugcuucugguaaaacuucugaaucuggugaauuacaugguuuaacuacugaagaagaauuuguugaagguauuuauaaaguugaaauugauacuaaaucuuauuggaaagcuuuagguauuucuccuuuucaugaacaugcugaaguuguuuuuacugcuaaugauucugguccucgucguuauacuauugcugcuuuauuaucuccuuauucuuauucuacuacugcuguuguuacuaauccuaaauag

SEQ ID NO: 132 - DNA, 441 bp, Artificial Sequence
  Synthetic: TTR BP_GCU ORF
atggcgtcgcaccgcctcctcctcctctgcctcgcgggcctcgtcttcgtctcggaggcgggccccacgggcacgggcgagtcgaagtgccccctcatggtcaaggtcctcgacgcggtccgcggctcgcccgcgatcaacgtcgcggtccacgtcttccgcaaggcggcggacgacacgtgggagcccttcgcgtcgggcaagacgtcggagtcgggcgagctccacggcctcacgacggaggaggagttcgtcgagggcatctacaaggtcgagatcgacacgaagtcgtactggaaggcgctcggcatctcgcccttccacgagcacgcggaggtcgtcttcacggcgaacgactcgggcccccgccgctacacgatcgcggcgctcctctcgccctactcgtactcgacgacggcggtcgtcacgaaccccaagtag

SEQ ID NO: 133 - DNA, 441 bp, Artificial Sequence
  Synthetic: TTR GP_BP_BS_GCU ORF
atggcgtcgcaccgcctcctcctcctctgcctcgccgggctcgtcttcgtcagcgaagccgggcccaccggcacgggtgagtcgaagtgccccctcatggtcaaggtcctcgacgctgtgcgcggctcgcctgcgatcaacgtcgctgtgcacgtcttccgcaaggctgccgacgacacgtgggagcccttcgcgtcgggcaagacgtcggagtcgggtgagctgcacggcctcacgacagaggaggagttcgtcgagggcatctacaaggtcgagatcgacacgaagtcgtactggaaggcactgggcatctcgcccttccacgagcacgcggaagtcgtcttcacggcgaacgactcgggcccccgccgctacacgatcgctgcactgctcagcccctactcgtactcgactacggctgtggtcacgaaccccaagtag

SEQ ID NO: 134 - DNA, 441 bp, Artificial Sequence
  Synthetic: TTR GS_BS_GCU ORF
atggcgagccaccgcctcctcctcctctgcctcgccgggctcgtcttcgtcagcgaagccgggcccaccggcacgggtgagagcaagtgccccctcatggtcaaggtcctcgacgctgtgcgcggcagccctgcgatcaacgtcgctgtgcacgtcttccgcaaggctgccgacgacacgtgggagcccttcgcgagcggcaagacgagcgagagcggtgagctgcacggcctcacgacagaggaggagttcgtcgagggcatctacaaggtcgagatcgacacgaagagctactggaaggcactgggcatcagccccttccacgagcacgcggaagtcgtcttcacggcgaacgacagcggcccccgccgctacacgatcgctgcactgctcagcccctacagctactcgactacggctgtggtcacgaaccccaagtag

SEQ ID NO: 135 - DNA, 441 bp, Artificial Sequence
  Synthetic: TTR GS_GCU ORF
atggcgagccaccgcctgctgctgctgtgcctggccgggctggtgttcgtcagcgaagccgggcccaccggcacgggtgagagcaagtgcccgctgatggtgaaggtgctggacgctgtgcgcggcagcccggcgatcaacgtggctgtgcacgtgttccgcaaggctgccgacgacacgtgggagccgttcgcgagcggcaagacgagcgagagcggtgagctgcacggcctgacgacagaggaggagttcgtggagggcatctacaaggtggagatcgacacgaagagctactggaaggcactgggcatcagcccgttccacgagcacgcggaagtcgtgttcacggcgaacgacagcggcccgcgccgctacacgatcgctgcactgctcagcccgtacagctactcgactacggctgtggtgacgaacccgaagtag

SEQ ID NO: 136 - RNA, 1257 nt, Artificial Sequence
  Synthetic: FAH BS_GCU
augagcuucauccccgucgcggaggacagcgacuuccccauccacaaccuccccuacggcgucuucagcacgcgcggcgacccccgcccccgcaucggcgucgcgaucggcgaccagauccucgaccucagcaucaucaagcaccucuucacgggccccguccucagcaagcaccaggacgucuucaaccagcccacgcucaacagcuucaugggccucggccaggcggcguggaaggaggcgcgcgucuuccuccagaaccuccucagcgucagccaggcgcgccuccgcgacgacacggagcuccgcaagugcgcguucaucagccaggcgagcgcgacgaugcaccuccccgcgacgaucggcgacuacacggacuucuacagcagccgccagcacgcgacgaacgucggcaucauguuccgcgacaaggagaacgcgcucaugcccaacuggcuccaccuccccgucggcuaccacggccgcgcgagcagcgucgucgucagcggcacgcccauccgccgccccaugggccagaugaagcccgacgacagcaagccccccgucuacggcgcgugcaagcuccucgacauggagcucgagauggcguucuucgucggccccggcaaccgccucggcgagcccauccccaucagcaaggcgcacgagcacaucuucggcaugguccucaugaacgacuggagcgcgcgcgacauccagaagugggaguacgucccccucggccccuuccucggcaagagcuucggcacgacggucagccccugggucguccccauggacgcgcucaugcccuucgcgguccccaaccccaagcaggacccccgcccccuccccuaccucugccacgacgagcccuacacguucgacaucaaccucagcgucaaccucaagggcgagggcaugagccaggcggcgacgaucugcaagagcaacuucaaguacauguacuggacgaugcuccagcagcucacgcaccacagcgucaacggcugcaaccuccgccccggcgaccuccucgcgagcggcacgaucagcggccccgagcccgagaacuucggcagcaugcucgagcucagcuggaagggcacgaagcccaucgaccucggcaacggccagacgcgcaaguuccuccucgacggcgacgaggucaucaucacgggcuacugccagggcgacggcuaccgcaucggcuucggccagugcgcgggcaagguccuccccgcgcuccuccccuag

SEQ ID NO: 137 - RNA, 1395 nt, Artificial Sequence
  Synthetic: GABRD BS_GCU
augagcgaggcgacgccccucgaccgcaacgacagcgagaacacgggcggccucaucagccgcccccaccccugggaccagagccccagcugcguccaggaggaccgcgcgaugaacgacaucggcgacuacgucggcagcaaccucgagaucagcuggcuccccaaccucgacggccucaucgcgggcuacgcgcgcaacuuccgccccggcaucggcggcccccccgucaacgucgcgcucgcgcucgaggucgcgagcaucgaccacaucagcgaggcgaacauggaguacacgaugacggucuuccuccaccagagcuggcgcgacagccgccucagcuacaaccacacgaacgagacgcucggccucgacagccgcuucgucgacaagcucuggcuccccgacacguucaucgucaacgcgaagagcgcgugguuccacgacgucacggucgagaacaagcucauccgccuccagcccgacggcgucauccucuacagcauccgcaucacgagcacggucgcgugcgacauggaccucgcgaaguaccccauggacgagcaggagugcaugcucgaccucgagagcuacggcuacagcagcgaggacaucgucuacuacuggagcgagagccaggagcacauccacggccucgacaagcuccagcucgcgcaguucacgaucacgagcuaccgcuucacgacggagcucaugaacuucaagagcgcgggccaguucccccgccucagccuccacuuccaccuccgccgcaaccgcggcgucuacaucauccagagcuacaugcccagcguccuccucgucgcgaugagcugggucagcuucuggaucagccaggcggcgguccccgcgcgcgucagccucggcaucacgacgguccucacgaugacgacgcucauggucagcgcgcgcagcagccucccccgcgcgagcgcgaucaaggcgcucgacgucuacuucuggaucugcuacgucuucgucuucgcggcgcucgucgaguacgcguucgcgcacuucaacgcggacuaccgcaagaagcagaaggcgaaggucaaggucagccgcccccgcgcggagauggacguccgcaacgcgaucguccucuucagccucagcgcggcgggcgucacgcaggagcucgcgaucagccgccgccagcgccgcguccccggcaaccucaugggcagcuaccgcagcgucggcgucgagacgggcgagacgaagaaggagggcgcggcgcgcagcggcggccagggcggcauccgcgcgcgccuccgccccaucgacgcggacacgaucgacaucuacgcgcgcgcggucuuccccgcggcguucgcggcggucaacgucaucuacugggcggcguacgcguag

SEQ ID NO: 138 - RNA, 951 nt, Artificial Sequence
  Synthetic: GAPDH BS_GCU
augggcaaggucaaggucggcgucaacggcuucggccgcaucggccgccucgucacgcgcgcggcguucaacagcggcaaggucgacaucgucgcgaucaacgaccccuucaucgaccucaacuacauggcggagaacggcaagcucgucaucaacggcaaccccaucacgaucuuccaggagcgcgaccccagcaagaucaaguggggcgacgcgggcgcggaguacgucgucgagagcacgggcgucuucacgacgauggagaaggcgggcgcgcaccuccagggcggcgcgaagcgcgucaucaucagcgcgcccagcgcggacgcgcccauguucgucaugggcgucaaccacgagaaguacgacaacagccucaagaucaucagcaacgcgagcugcacgacgaacugccucgcgccccucgcgaaggucauccacgacaacuucggcaucgucgagggccucaugacgacgguccacgcgaucacggcgacgcagaagacggucgacggccccagcggcaagcucuggcgcgacggccgcggcgcgcuccagaacaucauccccgcgagcacgggcgcggcgaaggcggucggcaaggucauccccgagcucaacggcaagcucacgggcauggcguuccgcguccccacggcgaacgucagcgucgucgaccucacgugccgccucgagaagcccgcgaaguacgacgacaucaagaaggucgucaagcaggcgagcgagggcccccucaagggcauccucggcuacacggagcaccaggucgucagcagcgacuucaacagcgacacgcacagcagcacguucgacgcgggcgcgggcaucgcgcucaacgaccacuucgucaagcucaucagcugguacgacaacgaguucggcuacagcaaccgcgucgucgaccucauggcgcacauggcgagcaaguag

SEQ ID NO: 139 - RNA, 1608 nt, Artificial Sequence
  Synthetic: GBA1 BS_GCU
auggaguucagcagccccagccgcgaggagugccccaagccccucagccgcgucagcaucauggcgggcagccucacgggccuccuccuccuccaggcggucagcugggcgagcggcgcgcgccccugcauccccaagagcuucggcuacagcagcgucgucugcgucugcaacgcgacguacugcgacagcuucgacccccccacguuccccgcgcucggcacguucagccgcuacgagagcacgcgcagcggccgccgcauggagcucagcaugggccccauccaggcgaaccacacgggcacgggccuccuccucacgcuccagcccgagcagaaguuccagaaggucaagggcuucggcggcgcgaugacggacgcggcggcgcucaacauccucgcgcucagcccccccgcgcagaaccuccuccucaagagcuacuucagcgaggagggcaucggcuacaacaucauccgcguccccauggcgagcugcgacuucagcauccgcacguacacguacgcggacacgcccgacgacuuccagcuccacaacuucagccuccccgaggaggacacgaagcucaagaucccccucauccaccgcgcgcuccagcucgcgcagcgccccgucagccuccucgcgagccccuggacgagccccacguggcucaagacgaacggcgcggucaacggcaagggcagccucaagggccagcccggcgacaucuaccaccagacgugggcgcgcuacuucgucaaguuccucgacgcguacgcggagcacaagcuccaguucugggcggucacggcggagaacgagcccagcgcgggccuccucagcggcuaccccuuccagugccucggcuucacgcccgagcaccagcgcgacuucaucgcgcgcgaccucggccccacgcucgcgaacagcacgcaccacaacguccgccuccucaugcucgacgaccagcgccuccuccucccccacugggcgaaggucguccucacggaccccgaggcggcgaaguacguccacggcaucgcgguccacugguaccucgacuuccucgcgcccgcgaaggcgacgcucggcgagacgcaccgccucuuccccaacacgaugcucuucgcgagcgaggcgugcgucggcagcaaguucugggagcagagcguccgccucggcagcugggaccgcggcaugcaguacagccacagcaucaucacgaaccuccucuaccacgucgucggcuggacggacuggaaccucgcgcucaaccccgagggcggccccaacuggguccgcaacuucgucgacagccccaucaucgucgacaucacgaaggacacguucuacaagcagcccauguucuaccaccucggccacuucagcaaguucauccccgagggcagccagcgcgucggccucgucgcgagccagaagaacgaccucgacgcggucgcgcucaugcaccccgacggcagcgcggucgucgucguccucaaccgcagcagcaaggacgucccccucacgaucaaggaccccgcggucggcuuccucgagacgaucagccccggcuacagcauccacacguaccucuggcgccgcuag

SEQ ID NO: 140 - RNA, 1287 nt, Artificial Sequence
  Synthetic: GLA BS_GCU
augcagcuccgcaaccccgagcuccaccucggcugcgcgcucgcgcuccgcuuccucgcgcucgucagcugggacauccccggcgcgcgcgcgcucgacaacggccucgcgcgcacgcccacgaugggcuggcuccacugggagcgcuucaugugcaaccucgacugccaggaggagcccgacagcugcaucagcgagaagcucuucauggagauggcggagcucauggucagcgagggcuggaaggacgcgggcuacgaguaccucugcaucgacgacugcuggauggcgccccagcgcgacagcgagggccgccuccaggcggacccccagcgcuucccccacggcauccgccagcucgcgaacuacguccacagcaagggccucaagcucggcaucuacgcggacgucggcaacaagacgugcgcgggcuuccccggcagcuucggcuacuacgacaucgacgcgcagacguucgcggacuggggcgucgaccuccucaaguucgacggcugcuacugcgacagccucgagaaccucgcggacggcuacaagcacaugagccucgcgcucaaccgcacgggccgcagcaucgucuacagcugcgaguggccccucuacauguggcccuuccagaagcccaacuacacggagauccgccaguacugcaaccacuggcgcaacuucgcggacaucgacgacagcuggaagagcaucaagagcauccucgacuggacgagcuucaaccaggagcgcaucgucgacgucgcgggccccggcggcuggaacgaccccgacaugcucgucaucggcaacuucggccucagcuggaaccagcaggucacgcagauggcgcucugggcgaucauggcggcgccccucuucaugagcaacgaccuccgccacaucagcccccaggcgaaggcgcuccuccaggacaaggacgucaucgcgaucaaccaggacccccucggcaagcagggcuaccagcuccgccagggcgacaacuucgaggucugggagcgcccccucagcggccucgcgugggcggucgcgaugaucaaccgccaggagaucggcggcccccgcagcuacacgaucgcggucgcgagccucggcaagggcgucgcgugcaaccccgcgugcuucaucacgcagcuccuccccgucaagcgcaagcucggcuucuacgaguggacgagccgccuccgcagccacaucaaccccacgggcacgguccuccuccagcucgagaacacgaugcagaugagccucaaggaccucuag

SEQ ID NO: 141 - RNA, 1062 nt, Artificial Sequence
  Synthetic: OTC BS_GCU
augcucuucaaccuccgcauccuccucaacaacgcggcguuccgcaacggccacaacuucaugguccgcaacuuccgcugcggccagccccuccagaacaagguccagcucaagggccgcgaccuccucacgcucaagaacuucacgggcgaggagaucaaguacaugcucuggcucagcgcggaccucaaguuccgcaucaagcagaagggcgaguaccucccccuccuccagggcaagagccucggcaugaucuucgagaagcgcagcacgcgcacgcgccucagcacggagacgggcuucgcgcuccucggcggccaccccugcuuccucacgacgcaggacauccaccucggcgucaacgagagccucacggacacggcgcgcguccucagcagcauggcggacgcgguccucgcgcgcgucuacaagcagagcgaccucgacacgcucgcgaaggaggcgagcauccccaucaucaacggccucagcgaccucuaccaccccauccagauccucgcggacuaccucacgcuccaggagcacuacagcagccucaagggccucacgcucagcuggaucggcgacggcaacaacauccuccacagcaucaugaugagcgcggcgaaguucggcaugcaccuccaggcggcgacgcccaagggcuacgagcccgacgcgagcgucacgaagcucgcggagcaguacgcgaaggagaacggcacgaagcuccuccucacgaacgacccccucgaggcggcgcacggcggcaacguccucaucacggacacguggaucagcaugggccaggaggaggagaagaagaagcgccuccaggcguuccagggcuaccaggucacgaugaagacggcgaaggucgcggcgagcgacuggacguuccuccacugccucccccgcaagcccgaggaggucgacgacgaggucuucuacagcccccgcagccucgucuuccccgaggcggagaaccgcaaguggacgaucauggcggucauggucagccuccucacggacuacagcccccagcuccagaagcccaaguag

SEQ ID NO: 142 - RNA, 1356 nt, Artificial Sequence
  Synthetic: PAH BS_GCU
augagcacggcgguccucgagaaccccggccucggccgcaagcucagcgacuucggccaggagacgagcuacaucgaggacaacugcaaccagaacggcgcgaucagccucaucuucagccucaaggaggaggucggcgcgcucgcgaagguccuccgccucuucgaggagaacgacgucaaccucacgcacaucgagagccgccccagccgccucaagaaggacgaguacgaguucuucacgcaccucgacaagcgcagccuccccgcgcucacgaacaucaucaagauccuccgccacgacaucggcgcgacgguccacgagcucagccgcgacaagaagaaggacacgguccccugguucccccgcacgauccaggagcucgaccgcuucgcgaaccagauccucagcuacggcgcggagcucgacgcggaccaccccggcuucaaggaccccgucuaccgcgcgcgccgcaagcaguucgcggacaucgcguacaacuaccgccacggccagcccaucccccgcgucgaguacauggaggaggagaagaagacguggggcacggucuucaagacgcucaagagccucuacaagacgcacgcgugcuacgaguacaaccacaucuucccccuccucgagaaguacugcggcuuccacgaggacaacaucccccagcucgaggacgucagccaguuccuccagacgugcacgggcuuccgccuccgccccgucgcgggccuccucagcagccgcgacuuccucggcggccucgcguuccgcgucuuccacugcacgcaguacauccgccacggcagcaagcccauguacacgcccgagcccgacaucugccacgagcuccucggccacgucccccucuucagcgaccgcagcuucgcgcaguucagccaggagaucggccucgcgagccucggcgcgcccgacgaguacaucgagaagcucgcgacgaucuacugguucacggucgaguucggccucugcaagcagggcgacagcaucaaggcguacggcgcgggccuccucagcagcuucggcgagcuccaguacugccucagcgagaagcccaagcuccucccccucgagcucgagaagacggcgauccagaacuacacggucacggaguuccagccccucuacuacgucgcggagagcuucaacgacgcgaaggagaagguccgcaacuucgcggcgacgaucccccgccccuucagcguccgcuacgaccccuacacgcagcgcaucgagguccucgacaacacgcagcagcucaagauccucgcggacagcaucaacagcgagaucggcauccucugcagcgcgcuccagaagaucuag

SEQ ID NO: 143 - RNA, 441 nt, Artificial Sequence
  Synthetic: TTR BS_GCU
auggcgagccaccgccuccuccuccucugccucgcgggccucgucuucgucagcgaggcgggccccacgggcacgggcgagagcaagugcccccucauggucaagguccucgacgcgguccgcggcagccccgcgaucaacgucgcgguccacgucuuccgcaaggcggcggacgacacgugggagcccuucgcgagcggcaagacgagcgagagcggcgagcuccacggccucacgacggaggaggaguucgucgagggcaucuacaaggucgagaucgacacgaagagcuacuggaaggcgcucggcaucagccccuuccacgagcacgcggaggucgucuucacggcgaacgacagcggcccccgccgcuacacgaucgcggcgcuccucagccccuacagcuacagcacgacggcggucgucacgaaccccaaguag

SEQ ID NO: 144-160 - skipped (17 entries)

SEQ ID NO: 161 - protein, 1379 aa, Artificial Sequence
  Synthetic: Cas9 nickase amino acid sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKV

SEQ ID NO: 162 - protein, 1379 aa, Artificial Sequence
  Synthetic: dCas9 amino acid sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKV

SEQ ID NO: 163 - protein, 7 aa, Artificial Sequence
  Synthetic: Exemplary NLS amino acid sequence
PKKKRKV

SEQ ID NO: 164 - protein, 9 aa, Artificial Sequence
  Synthetic: Exemplary NLS amino acid sequence
LAAKRSRTT

SEQ ID NO: 165 - protein, 9 aa, Artificial Sequence
  Synthetic: Exemplary NLS amino acid sequence
QAAKRSRTT

SEQ ID NO: 166 - protein, 10 aa, Artificial Sequence
  Synthetic: Exemplary NLS amino acid sequence
PAPAKRERTT

SEQ ID NO: 167 - protein, 9 aa, Artificial Sequence
  Synthetic: Exemplary NLS amino acid sequence
QAAKRPRTT

SEQ ID NO: 168 - protein, 9 aa, Artificial Sequence
  Synthetic: Exemplary NLS amino acid sequence
RAAKRPRTT

SEQ ID NO: 169 - protein, 11 aa, Artificial Sequence
  Synthetic: Exemplary NLS amino acid sequence
AAAKRSWSMAA

SEQ ID NO: 170 - protein, 11 aa, Artificial Sequence
  Synthetic: Exemplary NLS amino acid sequence
AAAKRVWSMAF

SEQ ID NO: 171 - protein, 11 aa, Artificial Sequence
  Synthetic: Exemplary NLS amino acid sequence
AAAKRSWSMAF

SEQ ID NO: 172 - protein, 10 aa, Artificial Sequence
  Synthetic: Exemplary NLS amino acid sequence
AAAKRKYFAA

SEQ ID NO: 173 - protein, 10 aa, Artificial Sequence
  Synthetic: Exemplary NLS amino acid sequence
RAAKRKAFAA

SEQ ID NO: 174 - protein, 10 aa, Artificial Sequence
  Synthetic: Exemplary NLS amino acid sequence
RAAKRKYFAV

SEQ ID NO: 175 - protein, 7 aa, Artificial Sequence
  Synthetic: Exemplary NLS amino acid sequence
PKKKRRV

SEQ ID NO: 176 - protein, 16 aa, Artificial Sequence
  Synthetic: Exemplary NLS amino acid sequence
KRPAATKKAGQAKKKK

SEQ ID NO: 177 - DNA, 50 bp, Artificial Sequence
  Synthetic: Exemplary 5 UTR
acatttgcttctgacacaactgtgttcactagcaacctcaaacagacacc

SEQ ID NO: 178 - DNA, 66 bp, Artificial Sequence
  Synthetic: Exemplary 5 UTR
cataaaccctggcgcgctcgcggcccggcactcttctggtccccacagactcagagagaacccacc

SEQ ID NO: 179 - DNA, 29 bp, Artificial Sequence
  Synthetic: Exemplary 5 UTR
aagctcagaataaacgctcaactttggcc

SEQ ID NO: 180 - DNA, 27 bp, Artificial Sequence
  Synthetic: Exemplary 5 UTR
cagggtcctgtggacagctcaccagct

SEQ ID NO: 181 - DNA, 61 bp, Artificial Sequence
  Synthetic: Exemplary 5 UTR
tcccgcagtcggcgtccagcggctctgcttgttcgtgtgtgtgtcgttgcaggccttattc

SEQ ID NO: 182 - DNA, 132 bp, Artificial Sequence
  Synthetic: Exemplary 3 UTR
gctcgctttcttgctgtccaatttctattaaaggttcctttgttccctaagtccaactactaaactgggggatattatgaagggccttgagcatctggattctgcctaataaaaaacatttattttcattgc

SEQ ID NO: 183 - DNA, 110 bp, Artificial Sequence
  Synthetic: Exemplary 3 UTR
gctggagcctcggtggccatgcttcttgccccttgggcctccccccagcccctcctccccttcctgcacccgtacccccgtggtctttgaataaagtctgagtgggcggc

SEQ ID NO: 184 - DNA, 130 bp, Artificial Sequence
  Synthetic: Exemplary 3 UTR
accagcctcaagaacacccgaatggagtctctaagctacataataccaacttacactttacaaaatgttgtcccccaaaatgtagccattcgtatctgctcctaataaaaagaaagtttcttcacattct

SEQ ID NO: 185 - DNA, 102 bp, Artificial Sequence
  Synthetic: Exemplary 3 UTR
ttgccagccatctgttgtttgcccctcccccgtgccttccttgaccctggaaggtgccactcccactgtcctttcctaataaaatgaggaaattgcatcgca

SEQ ID NO: 186 - DNA, 93 bp, Artificial Sequence
  Synthetic: Exemplary 3 UTR
gctgccttctgcggggcttgccttctggccatgcccttcttctctcccttgcacctgtacctcttggtctttgaataaagcctgagtaggaag

SEQ ID NO: 187 - RNA, 13 nt, Artificial Sequence
  Synthetic: Exemplary Kozak sequence
gccgccrccaugg

SEQ ID NO: 188 - RNA, 105 nt, Artificial Sequence
  Synthetic: Exemplary poly-A sequence
aaaaaaaaaaaaaaaaaaaaaaaaaaaaaagcgaaaaaaaaaaaaaaaaaaaaaaaaaaaaaaccgaaaaaaaaaaaaaaaaaaaaaaaaaaaaaaaaaaaaaaa

SEQ ID NO: 189 - RNA, 100 nt, Artificial Sequence
  Synthetic: Exemplary guide pattern
  modified_base (1)..(3): 2′-O-Me nucleotide
  modified_base (1)..(4): PS linkage
  misc_feature (1)..(20): n is a, c, g, or u
  modified_base (29)..(40): 2′-O-Me nucleotide
  modified_base (69)..(100): 2′-O-Me nucleotide
  modified_base (97)..(100): PS linkage
nnnnnnnnnnnnnnnnnnnnguuuuagagcuagaaauagcaaguuaaaauaaggcuaguccguuaucaacuugaaaaaguggcaccgagucggugcuuuu

SEQ ID NO: 190 - DNA, 42 bp, Artificial Sequence
  Synthetic: Exemplary 5 UTR
cagatcgcctggagacgccatccacgctgttttgacctccat

SEQ ID NO: 191 - DNA, 42 bp, Artificial Sequence
  Synthetic: Exemplary 5 UTR
agaagacaccgggaccgatccagcctccgcggccgggaacgg

SEQ ID NO: 192 - DNA, 42 bp, Artificial Sequence
  Synthetic: Exemplary 5 UTR
tgcattggaacgcggattccccgtgccaagagtgactcaccg

SEQ ID NO: 193 - RNA, 4423 nt, Artificial Sequence
  Synthetic: ORF32 mRNA
gggaagcucagaauaaacgcucaacuuuggccggaucugccaccauggacaagaaguacagcaucggacuggacaucggaacaaacagcguuggcugggcugugaucacagacgaauacaagguucccucaaagaaguucaagguccugggaaacacagacagacacagcaucaagaagaaucucaucggugcacugcuguucgacagcggugagacagcagaagccacaagacugaagagaacagcccgcagaagauacacaagaagaaagaacagaaucugcuaccugcaggagaucuucagcaacgaaauggcaaaggucgacgacagcuucuuccacagacuggaagaaagcuuccuggucgaagaagacaagaagcacgaaagacacccgaucuucggaaacaucgucgacgaagucgcauaccacgaaaaguaccccaccaucuaccaccugcggaagaagcuggucgacucgacugacaaggcagaccugcggcugaucuaccuggcacuggcacacaugauaaaguucagaggacacuuccugaucgaaggagaccugaacccugacaacagcgacgucgacaagcuguucauccagcugguccagaccuacaaccagcuguucgaagaaaacccgaucaacgcaagcggagucgacgcaaaggcaauccucagcgcccgccucagcaagagcagaagacuggaaaaucucaucgcacagcuuccaggugagaagaagaaugggcuguucggaaaucucaucgcacucagccugggacugacucccaacuucaagagcaacuucgaccuggcagaagacgcaaagcugcagcucagcaaggacaccuacgacgacgaccuggacaaucuccuggcacagaucggagaccaguacgcagaccuguuccuggcugccaagaaucucagcgacgcaauccugcucagcgacauccugcgggucaacacagagaucacaaaggcaccgcucagcgcaagcaugauaaagagauacgacgaacaccaccaggaccugacacugcugaaggcacuggucagacagcagcuuccagagaaguacaaggaaaucuucuucgaccagagcaagaauggguacgccggguacaucgacgguggugccagccaggaggaauucuacaaguucaucaagccgauccuggaaaagauggacggaacagaggagcugcuggucaagcugaacagggaggaccugcugcggaagcagagaacauucgacaaugggagcaucccccaccagauccaccugggugagcugcacgcaauccugcggagacaggaggacuucuacccguuccugaaggacaacagggagaagaucgaaaagauccugacauucagaauccccuacuacguuggcccgcuggcccgcggaaacagcagauucgcauggaugacaagaaagagcgaagaaacaaucacucccuggaacuucgaagaagucgucgacaagggugccagcgcacagagcuucaucgaaagaaugacaaacuucgacaagaaucuuccaaacgaaaagguccuuccaaagcacagccugcuguacgaauacuucacagucuacaacgagcugacaaaggucaaguacgucacagagggaaugagaaagccggcauuccucagcggugagcagaagaaggcaaucgucgaccugcuguucaagacaaacagaaaggucacagucaagcagcugaaggaagacuacuucaagaagaucgaaugcuucgacagcgucgaaaucagcggagucgaagacagauucaacgcaagccugggaaccuaccacgaccugcugaagaucaucaaggacaaggacuuccuggacaacgaagaaaacgaagacauccuggaagacaucguccugacacugacacuguucgaagacagggagaugauagaagaaagacugaagaccuacgcacaccuguucgacgacaaggucaugaagcagcugaagagaagaagauacacaggauggggaagacucagcagaaagcugaucaaugggauccgagacaagcagagcggaaagacaauccuggacuuccugaagagcgacggauucgcaaacagaaacuucaugcagcugauccacgacgacagccugacauucaaggaagacauccagaaggcacaggucagcggacagggcgacagccugcacgaacacaucgcaaaucucgccgggagcccggcaaucaagaaggggauccugcagacagucaaggucgucgacgagcuggucaaggucaugggaagacacaagccagagaacaucgucaucgaaauggccagggagaaccagacaacucaaaaggggcagaagaacagcagggagagaaugaagagaaucgaagaaggaaucaaggagcugggaagccagauccugaaggaacacccggucgaaaacacucaacugcagaacgaaaagcuguaccuguacuaccugcagaaugggcgagacauguacgucgaccaggagcuggacaucaacagacucagcgacuacgacgucgaccacaucguuccccagagcuuccugaaggacgacagcaucgacaacaagguccugacaagaagcgacaagaacagaggaaagagcgacaacguucccucagaagaagucgucaagaagaugaagaacuacuggagacagcugcugaacgcaaagcugaucacucaaagaaaguucgacaaucucacaaaggcagaaagagguggccucagcgagcuggacaaggccggguucaucaagagacagcuggucgaaacaagacagaucacaaagcacgucgcacagauccuggacagcagaaugaacacaaaguacgacgaaaacgacaagcugaucagggaagucaaggucaucacacugaagagcaagcuggucagcgacuucagaaaggacuuccaguucuacaaggucagggagaucaacaacuaccaccacgcacacgacgcauaccugaacgcugugguuggcacagcacugaucaagaaguacccgaagcuggaaagcgaauucgucuacggagacuacaaggucuacgacgucagaaagaugauagcaaagagcgaacaggagaucggaaaggcaacagcaaaguacuucuucuacagcaacaucaugaacuucuucaagacagagaucacacuggcaaauggugagaucagaaagagaccgcugaucgaaacaaauggugagacaggugagaucgucugggacaaggggcgagacuucgcaacagucagaaagguccucagcaugccgcaggugaacaucgucaagaagacagaaguccagacagguggcuucagcaaggaaagcauccuuccaaagagaaacagcgacaagcugaucgcccgcaagaaggacugggacccgaagaaguacgguggcuucgacagccccaccgucgcauacagcguccuggucgucgcaaaggucgaaaaggggaagagcaagaagcugaagagcgucaaggagcugcugggaaucacaaucauggaaagaagcagcuucgaaaagaacccgaucgacuuccuggaagccaagggguacaaggaagucaagaaggaccugaucaucaagcuuccaaaguacagccuguucgagcuggaaaaugggagaaagagaaugcuggcaagcgccggugagcugcagaaggggaacgagcuggcacuucccucaaaguacgucaacuuccuguaccuggcaagccacuacgaaaagcugaaggggagcccagaggacaacgaacagaagcagcuguucgucgaacagcacaagcacuaccuggacgaaaucaucgaacagaucagcgaauucagcaagagagucauccuggcagacgcaaaucucgacaagguccucagcgcauacaacaagcaccgagacaagccgaucagggagcaggccgaaaacaucauccaccuguucacacugacaaaucucggugccccggcugccuucaaguacuucgacacaacaaucgacagaaagagauacacaucgacuaaggaaguccuggacgcaacacugauccaccagagcaucacaggacuguacgaaacaagaaucgaccucagccagcuggguggcgacggugguggcagcccgaagaagaagagaaaggucuagcuagcaccagccucaagaacacccgaauggagucucuaagcuacauaauaccaacuuacacuuuacaaaauguugucccccaaaauguagccauucguaucugcuccuaauaaaaagaaaguuucuucacauucucucgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaucuag

SEQ ID NO: 194 - RNA, 4423 nt, Artificial Sequence
  Synthetic: ORF23 mRNA000112
gggaagcucagaauaaacgcucaacuuuggccggaucugccaccauggacaagaaguacagcaucggccuggacaucggcaccaacuccguuggcugggcugugaucaccgacgaguacaagguucccucaaagaaguucaaggugcugggcaacaccgaccggcacagcaucaagaagaaucucaucggugcacugcuguucgacuccggugagaccgccgaagccacccggcugaagcggaccgcccgccggcgguacacccggcggaagaaccggaucugcuaccugcaggagaucuucuccaacgagauggccaagguggacgacuccuucuuccaccggcuggaggaguccuuccugguggaggaggacaagaagcacgagcggcaccccaucuucggcaacaucguggacgaagucgccuaccacgagaaguaccccaccaucuaccaccugcggaagaagcugguggacucgacugacaaggccgaccugcggcugaucuaccuggcacuggcccacaugauaaaguuccggggccacuuccugaucgagggcgaccugaacccugacaacuccgacguggacaagcuguucauccagcuggugcagaccuacaaccagcuguucgaggagaaccccaucaacgccuccggcguggacgccaaggccauccucagcgcccgccucagcaagucccggcggcuggagaaucucaucgcccagcuuccaggugagaagaagaaugggcuguucggcaaucucaucgcacucagccugggccugacucccaacuucaaguccaacuucgaccuggccgaggacgccaagcugcagcucagcaaggacaccuacgacgacgaccuggacaaucuccuggcccagaucggcgaccaguacgccgaccuguuccuggcugccaagaaucucagcgacgccauccugcucagcgacauccugcgggugaacacagagaucaccaaggccccccucagcgccuccaugauaaagcgguacgacgagcaccaccaggaccugacccugcugaaggcacuggugcggcagcagcuuccagagaaguacaaggagaucuucuucgaccagagcaagaauggguacgccggguacaucgacgguggugccucccaggaggaguucuacaaguucaucaagcccauccuggagaagauggacggcacagaggagcugcuggugaagcugaacagggaggaccugcugcggaagcagcggaccuucgacaaugggagcaucccccaccagauccaccugggugagcugcacgccauccugcggcggcaggaggacuucuaccccuuccugaaggacaacagggagaagaucgagaagauccugaccuuccggauccccuacuacguuggcccccuggcccgcggcaacucccgguucgccuggaugacccggaaguccgaggagaccaucacucccuggaacuucgaggaagucguggacaagggugccuccgcccagagcuucaucgagcggaugaccaacuucgacaagaaucuuccaaacgagaaggugcuuccaaagcacucccugcuguacgaguacuucaccguguacaacgagcugaccaaggugaaguacgugacagagggcaugcggaagcccgccuuccucagcggugagcagaagaaggccaucguggaccugcuguucaagaccaaccggaaggugaccgugaagcagcugaaggaggacuacuucaagaagaucgagugcuucgacuccguggagaucuccggcguggaggaccgguucaacgccucccugggcaccuaccacgaccugcugaagaucaucaaggacaaggacuuccuggacaacgaggagaacgaggacauccuggaggacaucgugcugacccugacccuguucgaggacagggagaugauagaggagcggcugaagaccuacgcccaccuguucgacgacaaggugaugaagcagcugaagcggcggcgguacaccggcuggggccggcucagccggaagcugaucaaugggauccgagacaagcagagcggcaagaccauccuggacuuccugaaguccgacggcuucgccaaccggaacuucaugcagcugauccacgacgacucccugaccuucaaggaggacauccagaaggcccaggucagcggccagggcgacucccugcacgagcacaucgccaaucucgccgggucccccgccaucaagaaggggauccugcagaccgugaaggugguggacgagcuggugaaggugaugggccggcacaagccagagaacaucgugaucgagauggccagggagaaccagaccacucaaaaggggcagaagaacuccagggagcggaugaagcggaucgaggagggcaucaaggagcugggcucccagauccugaaggagcaccccguggagaacacucaacugcagaacgagaagcuguaccuguacuaccugcagaaugggcgagacauguacguggaccaggagcuggacaucaaccggcucagcgacuacgacguggaccacaucguuccccagagcuuccugaaggacgacagcaucgacaacaaggugcugacccgguccgacaagaaccggggcaaguccgacaacguucccucagaggaagucgugaagaagaugaagaacuacuggcggcagcugcugaacgccaagcugaucacucaacggaaguucgacaaucucaccaaggccgagcgggguggccucagcgagcuggacaaggccggguucaucaagcggcagcugguggagacccggcagaucaccaagcacguggcccagauccuggacucccggaugaacaccaaguacgacgagaacgacaagcugaucagggaagucaaggugaucacccugaaguccaagcuggucagcgacuuccggaaggacuuccaguucuacaaggugagggagaucaacaacuaccaccacgcccacgacgccuaccugaacgcugugguuggcaccgcacugaucaagaaguaccccaagcuggaguccgaguucguguacggcgacuacaagguguacgacgugcggaagaugauagccaaguccgagcaggagaucggcaaggccaccgccaaguacuucuucuacuccaacaucaugaacuucuucaagacagagaucacccuggccaauggugagauccggaagcggccccugaucgagaccaauggugagaccggugagaucgugugggacaaggggcgagacuucgccaccgugcggaaggugcucagcaugccccaggugaacaucgugaagaagacagaaguccagaccgguggcuucuccaaggagagcauccuuccaaagcggaacuccgacaagcugaucgcccgcaagaaggacugggaccccaagaaguacgguggcuucgacucccccaccguggccuacuccgugcuggugguggccaagguggagaaggggaaguccaagaagcugaaguccgugaaggagcugcugggcaucaccaucauggagcgguccuccuucgagaagaaccccaucgacuuccuggaagccaagggguacaaggaagucaagaaggaccugaucaucaagcuuccaaaguacucccuguucgagcuggagaaugggcggaagcggaugcuggccuccgccggugagcugcagaaggggaacgagcuggcacuucccucaaaguacgugaacuuccuguaccuggccucccacuacgagaagcugaagggguccccagaggacaacgagcagaagcagcuguucguggagcagcacaagcacuaccuggacgagaucaucgagcagaucuccgaguucuccaagcgggugauccuggccgacgccaaucucgacaaggugcucagcgccuacaacaagcaccgagacaagcccaucagggagcaggccgagaacaucauccaccuguucacccugaccaaucucggugcccccgcugccuucaaguacuucgacaccaccaucgaccggaagcgguacaccucgacuaaggaaguccuggacgccacccugauccaccagagcaucaccggccuguacgagacccggaucgaccucagccagcuggguggcgacggugguggcucccccaagaagaagcggaagguguagcuagcaccagccucaagaacacccgaauggagucucuaagcuacauaauaccaacuuacacuuuacaaaauguugucccccaaaauguagccauucguaucugcuccuaauaaaaagaaaguuucuucacauucucucgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaucuag

SEQ ID NO: 195 - RNA, 4553 nt, Artificial Sequence
  Synthetic: mRNA with SEQ ID No. 67 Cas9 ORF and 3′UTR XBG-XBG
gggaagcucagaauaaacgcucaacuuuggccggaucugccaccauggacaagaaguacuccaucggccuggacaucggcaccaacuccgugggcugggccgugaucaccgacgaguacaaggugcccuccaagaaguucaaggugcugggcaacaccgaccggcacuccaucaagaagaaccugaucggcgcccugcuguucgacuccggcgagaccgccgaggccacccggcugaagcggaccgcccggcggcgguacacccggcggaagaaccggaucugcuaccugcaggagaucuucuccaacgagauggccaagguggacgacuccuucuuccaccggcuggaggaguccuuccugguggaggaggacaagaagcacgagcggcaccccaucuucggcaacaucguggacgagguggccuaccacgagaaguaccccaccaucuaccaccugcggaagaagcugguggacuccaccgacaaggccgaccugcggcugaucuaccuggcccuggcccacaugaucaaguuccggggccacuuccugaucgagggcgaccugaaccccgacaacuccgacguggacaagcuguucauccagcuggugcagaccuacaaccagcuguucgaggagaaccccaucaacgccuccggcguggacgccaaggccauccuguccgcccggcuguccaagucccggcggcuggagaaccugaucgcccagcugcccggcgagaagaagaacggccuguucggcaaccugaucgcccugucccugggccugacccccaacuucaaguccaacuucgaccuggccgaggacgccaagcugcagcuguccaaggacaccuacgacgacgaccuggacaaccugcuggcccagaucggcgaccaguacgccgaccuguuccuggccgccaagaaccuguccgacgccauccugcuguccgacauccugcgggugaacaccgagaucaccaaggccccccuguccgccuccaugaucaagcgguacgacgagcaccaccaggaccugacccugcugaaggcccuggugcggcagcagcugcccgagaaguacaaggagaucuucuucgaccaguccaagaacggcuacgccggcuacaucgacggcggcgccucccaggaggaguucuacaaguucaucaagcccauccuggagaagauggacggcaccgaggagcugcuggugaagcugaaccgggaggaccugcugcggaagcagcggaccuucgacaacggcuccaucccccaccagauccaccugggcgagcugcacgccauccugcggcggcaggaggacuucuaccccuuccugaaggacaaccgggagaagaucgagaagauccugaccuuccggauccccuacuacgugggcccccuggcccggggcaacucccgguucgccuggaugacccggaaguccgaggagaccaucacccccuggaacuucgaggaggugguggacaagggcgccuccgcccaguccuucaucgagcggaugaccaacuucgacaagaaccugcccaacgagaaggugcugcccaagcacucccugcuguacgaguacuucaccguguacaacgagcugaccaaggugaaguacgugaccgagggcaugcggaagcccgccuuccuguccggcgagcagaagaaggccaucguggaccugcuguucaagaccaaccggaaggugaccgugaagcagcugaaggaggacuacuucaagaagaucgagugcuucgacuccguggagaucuccggcguggaggaccgguucaacgccucccugggcaccuaccacgaccugcugaagaucaucaaggacaaggacuuccuggacaacgaggagaacgaggacauccuggaggacaucgugcugacccugacccuguucgaggaccgggagaugaucgaggagcggcugaagaccuacgcccaccuguucgacgacaaggugaugaagcagcugaagcggcggcgguacaccggcuggggccggcugucccggaagcugaucaacggcauccgggacaagcaguccggcaagaccauccuggacuuccugaaguccgacggcuucgccaaccggaacuucaugcagcugauccacgacgacucccugaccuucaaggaggacauccagaaggcccagguguccggccagggcgacucccugcacgagcacaucgccaaccuggccggcucccccgccaucaagaagggcauccugcagaccgugaaggugguggacgagcuggugaaggugaugggccggcacaagcccgagaacaucgugaucgagauggcccgggagaaccagaccacccagaagggccagaagaacucccgggagcggaugaagcggaucgaggagggcaucaaggagcugggcucccagauccugaaggagcaccccguggagaacacccagcugcagaacgagaagcuguaccuguacuaccugcagaacggccgggacauguacguggaccaggagcuggacaucaaccggcuguccgacuacgacguggaccacaucgugccccaguccuuccugaaggacgacuccaucgacaacaaggugcugacccgguccgacaagaaccggggcaaguccgacaacgugcccuccgaggagguggugaagaagaugaagaacuacuggcggcagcugcugaacgccaagcugaucacccagcggaaguucgacaaccugaccaaggccgagcggggcggccuguccgagcuggacaaggccggcuucaucaagcggcagcugguggagacccggcagaucaccaagcacguggcccagauccuggacucccggaugaacaccaaguacgacgagaacgacaagcugauccgggaggugaaggugaucacccugaaguccaagcugguguccgacuuccggaaggacuuccaguucuacaaggugcgggagaucaacaacuaccaccacgcccacgacgccuaccugaacgccguggugggcaccgcccugaucaagaaguaccccaagcuggaguccgaguucguguacggcgacuacaagguguacgacgugcggaagaugaucgccaaguccgagcaggagaucggcaaggccaccgccaaguacuucuucuacuccaacaucaugaacuucuucaagaccgagaucacccuggccaacggcgagauccggaagcggccccugaucgagaccaacggcgagaccggcgagaucgugugggacaagggccgggacuucgccaccgugcggaaggugcuguccaugccccaggugaacaucgugaagaagaccgaggugcagaccggcggcuucuccaaggaguccauccugcccaagcggaacuccgacaagcugaucgcccggaagaaggacugggaccccaagaaguacggcggcuucgacucccccaccguggccuacuccgugcuggugguggccaagguggagaagggcaaguccaagaagcugaaguccgugaaggagcugcugggcaucaccaucauggagcgguccuccuucgagaagaaccccaucgacuuccuggaggccaagggcuacaaggaggugaagaaggaccugaucaucaagcugcccaaguacucccuguucgagcuggagaacggccggaagcggaugcuggccuccgccggcgagcugcagaagggcaacgagcuggcccugcccuccaaguacgugaacuuccuguaccuggccucccacuacgagaagcugaagggcucccccgaggacaacgagcagaagcagcuguucguggagcagcacaagcacuaccuggacgagaucaucgagcagaucuccgaguucuccaagcgggugauccuggccgacgccaaccuggacaaggugcuguccgccuacaacaagcaccgggacaagcccauccgggagcaggccgagaacaucauccaccuguucacccugaccaaccugggcgcccccgccgccuucaaguacuucgacaccaccaucgaccggaagcgguacaccuccaccaaggaggugcuggacgccacccugauccaccaguccaucaccggccuguacgagacccggaucgaccugucccagcugggcggcgacggcggcggcucccccaagaagaagcggaaggugugacuagcaccagccucaagaacacccgaauggagucucuaagcuacauaauaccaacuuacacuuuacaaaauguugucccccaaaauguagccauucguaucugcuccuaauaaaaagaaaguuucuucacauucuaccagccucaagaacacccgaauggagucucuaagcuacauaauaccaacuuacacuuuacaaaauguugucccccaaaauguagccauucguaucugcuccuaauaaaaagaaaguuucuucacauucucucgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaucuag

SEQ ID NO: 196 - RNA, 4566 nt, Artificial Sequence
  Synthetic: mRNA with SEQ ID No. 67 Cas9 ORF and 3′UTR AES-mtRNR1
gggaagcucagaauaaacgcucaacuuuggccggaucugccaccauggacaagaaguacuccaucggccuggacaucggcaccaacuccgugggcugggccgugaucaccgacgaguacaaggugcccuccaagaaguucaaggugcugggcaacaccgaccggcacuccaucaagaagaaccugaucggcgcccugcuguucgacuccggcgagaccgccgaggccacccggcugaagcggaccgcccggcggcgguacacccggcggaagaaccggaucugcuaccugcaggagaucuucuccaacgagauggccaagguggacgacuccuucuuccaccggcuggaggaguccuuccugguggaggaggacaagaagcacgagcggcaccccaucuucggcaacaucguggacgagguggccuaccacgagaaguaccccaccaucuaccaccugcggaagaagcugguggacuccaccgacaaggccgaccugcggcugaucuaccuggcccuggcccacaugaucaaguuccggggccacuuccugaucgagggcgaccugaaccccgacaacuccgacguggacaagcuguucauccagcuggugcagaccuacaaccagcuguucgaggagaaccccaucaacgccuccggcguggacgccaaggccauccuguccgcccggcuguccaagucccggcggcuggagaaccugaucgcccagcugcccggcgagaagaagaacggccuguucggcaaccugaucgcccugucccugggccugacccccaacuucaaguccaacuucgaccuggccgaggacgccaagcugcagcuguccaaggacaccuacgacgacgaccuggacaaccugcuggcccagaucggcgaccaguacgccgaccuguuccuggccgccaagaaccuguccgacgccauccugcuguccgacauccugcgggugaacaccgagaucaccaaggccccccuguccgccuccaugaucaagcgguacgacgagcaccaccaggaccugacccugcugaaggcccuggugcggcagcagcugcccgagaaguacaaggagaucuucuucgaccaguccaagaacggcuacgccggcuacaucgacggcggcgccucccaggaggaguucuacaaguucaucaagcccauccuggagaagauggacggcaccgaggagcugcuggugaagcugaaccgggaggaccugcugcggaagcagcggaccuucgacaacggcuccaucccccaccagauccaccugggcgagcugcacgccauccugcggcggcaggaggacuucuaccccuuccugaaggacaaccgggagaagaucgagaagauccugaccuuccggauccccuacuacgugggcccccuggcccggggcaacucccgguucgccuggaugacccggaaguccgaggagaccaucacccccuggaacuucgaggaggugguggacaagggcgccuccgcccaguccuucaucgagcggaugaccaacuucgacaagaaccugcccaacgagaaggugcugcccaagcacucccugcuguacgaguacuucaccguguacaacgagcugaccaaggugaaguacgugaccgagggcaugcggaagcccgccuuccuguccggcgagcagaagaaggccaucguggaccugcuguucaagaccaaccggaaggugaccgugaagcagcugaaggaggacuacuucaagaagaucgagugcuucgacuccguggagaucuccggcguggaggaccgguucaacgccucccugggcaccuaccacgaccugcugaagaucaucaaggacaaggacuuccuggacaacgaggagaacgaggacauccuggaggacaucgugcugacccugacccuguucgaggaccgggagaugaucgaggagcggcugaagaccuacgcccaccuguucgacgacaaggugaugaagcagcugaagcggcggcgguacaccggcuggggccggcugucccggaagcugaucaacggcauccgggacaagcaguccggcaagaccauccuggacuuccugaaguccgacggcuucgccaaccggaacuucaugcagcugauccacgacgacucccugaccuucaaggaggacauccagaaggcccagguguccggccagggcgacucccugcacgagcacaucgccaaccuggccggcucccccgccaucaagaagggcauccugcagaccgugaaggugguggacgagcuggugaaggugaugggccggcacaagcccgagaacaucgugaucgagauggcccgggagaaccagaccacccagaagggccagaagaacucccgggagcggaugaagcggaucgaggagggcaucaaggagcugggcucccagauccugaaggagcaccccguggagaacacccagcugcagaacgagaagcuguaccuguacuaccugcagaacggccgggacauguacguggaccaggagcuggacaucaaccggcuguccgacuacgacguggaccacaucgugccccaguccuuccugaaggacgacuccaucgacaacaaggugcugacccgguccgacaagaaccggggcaaguccgacaacgugcccuccgaggagguggugaagaagaugaagaacuacuggcggcagcugcugaacgccaagcugaucacccagcggaaguucgacaaccugaccaaggccgagcggggcggccuguccgagcuggacaaggccggcuucaucaagcggcagcugguggagacccggcagaucaccaagcacguggcccagauccuggacucccggaugaacaccaaguacgacgagaacgacaagcugauccgggaggugaaggugaucacccugaaguccaagcugguguccgacuuccggaaggacuuccaguucuacaaggugcgggagaucaacaacuaccaccacgcccacgacgccuaccugaacgccguggugggcaccgcccugaucaagaaguaccccaagcuggaguccgaguucguguacggcgacuacaagguguacgacgugcggaagaugaucgccaaguccgagcaggagaucggcaaggccaccgccaaguacuucuucuacuccaacaucaugaacuucuucaagaccgagaucacccuggccaacggcgagauccggaagcggccccugaucgagaccaacggcgagaccggcgagaucgugugggacaagggccgggacuucgccaccgugcggaaggugcuguccaugccccaggugaacaucgugaagaagaccgaggugcagaccggcggcuucuccaaggaguccauccugcccaagcggaacuccgacaagcugaucgcccggaagaaggacugggaccccaagaaguacggcggcuucgacucccccaccguggccuacuccgugcuggugguggccaagguggagaagggcaaguccaagaagcugaaguccgugaaggagcugcugggcaucaccaucauggagcgguccuccuucgagaagaaccccaucgacuuccuggaggccaagggcuacaaggaggugaagaaggaccugaucaucaagcugcccaaguacucccuguucgagcuggagaacggccggaagcggaugcuggccuccgccggcgagcugcagaagggcaacgagcuggcccugcccuccaaguacgugaacuuccuguaccuggccucccacuacgagaagcugaagggcucccccgaggacaacgagcagaagcagcuguucguggagcagcacaagcacuaccuggacgagaucaucgagcagaucuccgaguucuccaagcgggugauccuggccgacgccaaccuggacaaggugcuguccgccuacaacaagcaccgggacaagcccauccgggagcaggccgagaacaucauccaccuguucacccugaccaaccugggcgcccccgccgccuucaaguacuucgacaccaccaucgaccggaagcgguacaccuccaccaaggaggugcuggacgccacccugauccaccaguccaucaccggccuguacgagacccggaucgaccugucccagcugggcggcgacggcggcggcucccccaagaagaagcggaaggugugacugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacacccucgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaucuag

SEQ ID NO: 197 - RNA, 4566 nt, Artificial Sequence
  Synthetic: mRNA with SEQ ID No. 67 Cas9 ORF and 3′UTR mtRNR1-AES
gggaagcucagaauaaacgcucaacuuuggccggaucugccaccauggacaagaaguacuccaucggccuggacaucggcaccaacuccgugggcugggccgugaucaccgacgaguacaaggugcccuccaagaaguucaaggugcugggcaacaccgaccggcacuccaucaagaagaaccugaucggcgcccugcuguucgacuccggcgagaccgccgaggccacccggcugaagcggaccgcccggcggcgguacacccggcggaagaaccggaucugcuaccugcaggagaucuucuccaacgagauggccaagguggacgacuccuucuuccaccggcuggaggaguccuuccugguggaggaggacaagaagcacgagcggcaccccaucuucggcaacaucguggacgagguggccuaccacgagaaguaccccaccaucuaccaccugcggaagaagcugguggacuccaccgacaaggccgaccugcggcugaucuaccuggcccuggcccacaugaucaaguuccggggccacuuccugaucgagggcgaccugaaccccgacaacuccgacguggacaagcuguucauccagcuggugcagaccuacaaccagcuguucgaggagaaccccaucaacgccuccggcguggacgccaaggccauccuguccgcccggcuguccaagucccggcggcuggagaaccugaucgcccagcugcccggcgagaagaagaacggccuguucggcaaccugaucgcccugucccugggccugacccccaacuucaaguccaacuucgaccuggccgaggacgccaagcugcagcuguccaaggacaccuacgacgacgaccuggacaaccugcuggcccagaucggcgaccaguacgccgaccuguuccuggccgccaagaaccuguccgacgccauccugcuguccgacauccugcgggugaacaccgagaucaccaaggccccccuguccgccuccaugaucaagcgguacgacgagcaccaccaggaccugacccugcugaaggcccuggugcggcagcagcugcccgagaaguacaaggagaucuucuucgaccaguccaagaacggcuacgccggcuacaucgacggcggcgccucccaggaggaguucuacaaguucaucaagcccauccuggagaagauggacggcaccgaggagcugcuggugaagcugaaccgggaggaccugcugcggaagcagcggaccuucgacaacggcuccaucccccaccagauccaccugggcgagcugcacgccauccugcggcggcaggaggacuucuaccccuuccugaaggacaaccgggagaagaucgagaagauccugaccuuccggauccccuacuacgugggcccccuggcccggggcaacucccgguucgccuggaugacccggaaguccgaggagaccaucacccccuggaacuucgaggaggugguggacaagggcgccuccgcccaguccuucaucgagcggaugaccaacuucgacaagaaccugcccaacgagaaggugcugcccaagcacucccugcuguacgaguacuucaccguguacaacgagcugaccaaggugaaguacgugaccgagggcaugcggaagcccgccuuccuguccggcgagcagaagaaggccaucguggaccugcuguucaagaccaaccggaaggugaccgugaagcagcugaaggaggacuacuucaagaagaucgagugcuucgacuccguggagaucuccggcguggaggaccgguucaacgccucccugggcaccuaccacgaccugcugaagaucaucaaggacaaggacuuccuggacaacgaggagaacgaggacauccuggaggacaucgugcugacccugacccuguucgaggaccgggagaugaucgaggagcggcugaagaccuacgcccaccuguucgacgacaaggugaugaagcagcugaagcggcggcgguacaccggcuggggccggcugucccggaagcugaucaacggcauccgggacaagcaguccggcaagaccauccuggacuuccugaaguccgacggcuucgccaaccggaacuucaugcagcugauccacgacgacucccugaccuucaaggaggacauccagaaggcccagguguccggccagggcgacucccugcacgagcacaucgccaaccuggccggcucccccgccaucaagaagggcauccugcagaccgugaaggugguggacgagcuggugaaggugaugggccggcacaagcccgagaacaucgugaucgagauggcccgggagaaccagaccacccagaagggccagaagaacucccgggagcggaugaagcggaucgaggagggcaucaaggagcugggcucccagauccugaaggagcaccccguggagaacacccagcugcagaacgagaagcuguaccuguacuaccugcagaacggccgggacauguacguggaccaggagcuggacaucaaccggcuguccgacuacgacguggaccacaucgugccccaguccuuccugaaggacgacuccaucgacaacaaggugcugacccgguccgacaagaaccggggcaaguccgacaacgugcccuccgaggagguggugaagaagaugaagaacuacuggcggcagcugcugaacgccaagcugaucacccagcggaaguucgacaaccugaccaaggccgagcggggcggccuguccgagcuggacaaggccggcuucaucaagcggcagcugguggagacccggcagaucaccaagcacguggcccagauccuggacucccggaugaacaccaaguacgacgagaacgacaagcugauccgggaggugaaggugaucacccugaaguccaagcugguguccgacuuccggaaggacuuccaguucuacaaggugcgggagaucaacaacuaccaccacgcccacgacgccuaccugaacgccguggugggcaccgcccugaucaagaaguaccccaagcuggaguccgaguucguguacggcgacuacaagguguacgacgugcggaagaugaucgccaaguccgagcaggagaucggcaaggccaccgccaaguacuucuucuacuccaacaucaugaacuucuucaagaccgagaucacccuggccaacggcgagauccggaagcggccccugaucgagaccaacggcgagaccggcgagaucgugugggacaagggccgggacuucgccaccgugcggaaggugcuguccaugccccaggugaacaucgugaagaagaccgaggugcagaccggcggcuucuccaaggaguccauccugcccaagcggaacuccgacaagcugaucgcccggaagaaggacugggaccccaagaaguacggcggcuucgacucccccaccguggccuacuccgugcuggugguggccaagguggagaagggcaaguccaagaagcugaaguccgugaaggagcugcugggcaucaccaucauggagcgguccuccuucgagaagaaccccaucgacuuccuggaggccaagggcuacaaggaggugaagaaggaccugaucaucaagcugcccaaguacucccuguucgagcuggagaacggccggaagcggaugcuggccuccgccggcgagcugcagaagggcaacgagcuggcccugcccuccaaguacgugaacuuccuguaccuggccucccacuacgagaagcugaagggcucccccgaggacaacgagcagaagcagcuguucguggagcagcacaagcacuaccuggacgagaucaucgagcagaucuccgaguucuccaagcgggugauccuggccgacgccaaccuggacaaggugcuguccgccuacaacaagcaccgggacaagcccauccgggagcaggccgagaacaucauccaccuguucacccugaccaaccugggcgcccccgccgccuucaaguacuucgacaccaccaucgaccggaagcgguacaccuccaccaaggaggugcuggacgccacccugauccaccaguccaucaccggccuguacgagacccggaucgaccugucccagcugggcggcgacggcggcggcucccccaagaagaagcggaaggugugacaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacacccugguacugcaugcacgcaaugcuagcugccccuuucccguccuggguaccccgagucucccccgaccucgggucccagguaugcucccaccuccaccugccccacucaccaccucugcuaguuccagacaccucccucgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaucuag

SEQ ID NO: 198 - skipped

SEQ ID NO: 199 - RNA, 4560 nt, Artificial Sequence
  Synthetic: mRNA with SEQ ID No. 46 Cas9 ORF and 3′UTR XBG-XBG
gggaagcucagaauaaacgcucaacuuuggccggaucugccaccauggacaagaaguacagcaucggccuggacaucggcaccaacuccguuggcugggcugugaucaccgacgaguacaagguucccucaaagaaguucaaggugcugggcaacaccgaccggcacagcaucaagaagaaucucaucggugcacugcuguucgacuccggugagaccgccgaagccacccggcugaagcggaccgcccgccggcgguacacccggcggaagaaccggaucugcuaccugcaggagaucuucuccaacgagauggccaagguggacgacuccuucuuccaccggcuggaggaguccuuccugguggaggaggacaagaagcacgagcggcaccccaucuucggcaacaucguggacgaagucgccuaccacgagaaguaccccaccaucuaccaccugcggaagaagcugguggacucgacugacaaggccgaccugcggcugaucuaccuggcacuggcccacaugauaaaguuccggggccacuuccugaucgagggcgaccugaacccugacaacuccgacguggacaagcuguucauccagcuggugcagaccuacaaccagcuguucgaggagaaccccaucaacgccuccggcguggacgccaaggccauccucagcgcccgccucagcaagucccggcggcuggagaaucucaucgcccagcuuccaggugagaagaagaaugggcuguucggcaaucucaucgcacucagccugggccugacucccaacuucaaguccaacuucgaccuggccgaggacgccaagcugcagcucagcaaggacaccuacgacgacgaccuggacaaucuccuggcccagaucggcgaccaguacgccgaccuguuccuggcugccaagaaucucagcgacgccauccugcucagcgacauccugcgggugaacacagagaucaccaaggccccccucagcgccuccaugauaaagcgguacgacgagcaccaccaggaccugacccugcugaaggcacuggugcggcagcagcuuccagagaaguacaaggagaucuucuucgaccagagcaagaauggguacgccggguacaucgacgguggugccucccaggaggaguucuacaaguucaucaagcccauccuggagaagauggacggcacagaggagcugcuggugaagcugaacagggaggaccugcugcggaagcagcggaccuucgacaaugggagcaucccccaccagauccaccugggugagcugcacgccauccugcggcggcaggaggacuucuaccccuuccugaaggacaacagggagaagaucgagaagauccugaccuuccggauccccuacuacguuggcccccuggcccgcggcaacucccgguucgccuggaugacccggaaguccgaggagaccaucacucccuggaacuucgaggaagucguggacaagggugccuccgcccagagcuucaucgagcggaugaccaacuucgacaagaaucuuccaaacgagaaggugcuuccaaagcacucccugcuguacgaguacuucaccguguacaacgagcugaccaaggugaaguacgugacagagggcaugcggaagcccgccuuccucagcggugagcagaagaaggccaucguggaccugcuguucaagaccaaccggaaggugaccgugaagcagcugaaggaggacuacuucaagaagaucgagugcuucgacuccguggagaucuccggcguggaggaccgguucaacgccucccugggcaccuaccacgaccugcugaagaucaucaaggacaaggacuuccuggacaacgaggagaacgaggacauccuggaggacaucgugcugacccugacccuguucgaggacagggagaugauagaggagcggcugaagaccuacgcccaccuguucgacgacaaggugaugaagcagcugaagcggcggcgguacaccggcuggggccggcucagccggaagcugaucaaugggauccgagacaagcagagcggcaagaccauccuggacuuccugaaguccgacggcuucgccaaccggaacuucaugcagcugauccacgacgacucccugaccuucaaggaggacauccagaaggcccaggucagcggccagggcgacucccugcacgagcacaucgccaaucucgccgggucccccgccaucaagaaggggauccugcagaccgugaaggugguggacgagcuggugaaggugaugggccggcacaagccagagaacaucgugaucgagauggccagggagaaccagaccacucaaaaggggcagaagaacuccagggagcggaugaagcggaucgaggagggcaucaaggagcugggcucccagauccugaaggagcaccccguggagaacacucaacugcagaacgagaagcuguaccuguacuaccugcagaaugggcgagacauguacguggaccaggagcuggacaucaaccggcucagcgacuacgacguggaccacaucguuccccagagcuuccugaaggacgacagcaucgacaacaaggugcugacccgguccgacaagaaccggggcaaguccgacaacguucccucagaggaagucgugaagaagaugaagaacuacuggcggcagcugcugaacgccaagcugaucacucaacggaaguucgacaaucucaccaaggccgagcgggguggccucagcgagcuggacaaggccggguucaucaagcggcagcugguggagacccggcagaucaccaagcacguggcccagauccuggacucccggaugaacaccaaguacgacgagaacgacaagcugaucagggaagucaaggugaucacccugaaguccaagcuggucagcgacuuccggaaggacuuccaguucuacaaggugagggagaucaacaacuaccaccacgcccacgacgccuaccugaacgcugugguuggcaccgcacugaucaagaaguaccccaagcuggaguccgaguucguguacggcgacuacaagguguacgacgugcggaagaugauagccaaguccgagcaggagaucggcaaggccaccgccaaguacuucuucuacuccaacaucaugaacuucuucaagacagagaucacccuggccaauggugagauccggaagcggccccugaucgagaccaauggugagaccggugagaucgugugggacaaggggcgagacuucgccaccgugcggaaggugcucagcaugccccaggugaacaucgugaagaagacagaaguccagaccgguggcuucuccaaggagagcauccuuccaaagcggaacuccgacaagcugaucgcccgcaagaaggacugggaccccaagaaguacgguggcuucgacucccccaccguggccuacuccgugcuggugguggccaagguggagaaggggaaguccaagaagcugaaguccgugaaggagcugcugggcaucaccaucauggagcgguccuccuucgagaagaaccccaucgacuuccuggaagccaagggguacaaggaagucaagaaggaccugaucaucaagcuuccaaaguacucccuguucgagcuggagaaugggcggaagcggaugcuggccuccgccggugagcugcagaaggggaacgagcuggcacuucccucaaaguacgugaacuuccuguaccuggccucccacuacgagaagcugaagggguccccagaggacaacgagcagaagcagcuguucguggagcagcacaagcacuaccuggacgagaucaucgagcagaucuccgaguucuccaagcgggugauccuggccgacgccaaucucgacaaggugcucagcgccuacaacaagcaccgagacaagcccaucagggagcaggccgagaacaucauccaccuguucacccugaccaaucucggugcccccgcugccuucaaguacuucgacaccaccaucgaccggaagcgguacaccucgacuaaggaaguccuggacgccacccugauccaccagagcaucaccggccuguacgagacccggaucgaccucagccagcuggguggcgacggugguggcucccccaagaagaagcggaagguguagcuagcaccagccucaagaacacccgaauggagucucuaagcuacauaauaccaacuuacacuuuacaaaauguugucccccaaaauguagccauucguaucugcuccuaauaaaaagaaaguuucuucacauucuaccagccucaagaacacccgaauggagucucuaagcuacauaauaccaacuuacacuuuacaaaauguugucccccaaaauguagccauucguaucugcuccuaauaaaaagaaaguuucuucacauucucucgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaucuag

SEQ ID NO: 200 - RNA, 4573 nt, Artificial Sequence
  Synthetic: mRNA with SEQ ID No. 46 Cas9 ORF and 3′UTR AES-mtRNR1
gggaagcucagaauaaacgcucaacuuuggccggaucugccaccauggacaagaaguacagcaucggccuggacaucggcaccaacuccguuggcugggcugugaucaccgacgaguacaagguucccucaaagaaguucaaggugcugggcaacaccgaccggcacagcaucaagaagaaucucaucggugcacugcuguucgacuccggugagaccgccgaagccacccggcugaagcggaccgcccgccggcgguacacccggcggaagaaccggaucugcuaccugcaggagaucuucuccaacgagauggccaagguggacgacuccuucuuccaccggcuggaggaguccuuccugguggaggaggacaagaagcacgagcggcaccccaucuucggcaacaucguggacgaagucgccuaccacgagaaguaccccaccaucuaccaccugcggaagaagcugguggacucgacugacaaggccgaccugcggcugaucuaccuggcacuggcccacaugauaaaguuccggggccacuuccugaucgagggcgaccugaacccugacaacuccgacguggacaagcuguucauccagcuggugcagaccuacaaccagcuguucgaggagaaccccaucaacgccuccggcguggacgccaaggccauccucagcgcccgccucagcaagucccggcggcuggagaaucucaucgcccagcuuccaggugagaagaagaaugggcuguucggcaaucucaucgcacucagccugggccugacucccaacuucaaguccaacuucgaccuggccgaggacgccaagcugcagcucagcaaggacaccuacgacgacgaccuggacaaucuccuggcccagaucggcgaccaguacgccgaccuguuccuggcugccaagaaucucagcgacgccauccugcucagcgacauccugcgggugaacacagagaucaccaaggccccccucagcgccuccaugauaaagcgguacgacgagcaccaccaggaccugacccugcugaaggcacuggugcggcagcagcuuccagagaaguacaaggagaucuucuucgaccagagcaagaauggguacgccggguacaucgacgguggugccucccaggaggaguucuacaaguucaucaagcccauccuggagaagauggacggcacagaggagcugcuggugaagcugaacagggaggaccugcugcggaagcagcggaccuucgacaaugggagcaucccccaccagauccaccugggugagcugcacgccauccugcggcggcaggaggacuucuaccccuuccugaaggacaacagggagaagaucgagaagauccugaccuuccggauccccuacuacguuggcccccuggcccgcggcaacucccgguucgccuggaugacccggaaguccgaggagaccaucacucccuggaacuucgaggaagucguggacaagggugccuccgcccagagcuucaucgagcggaugaccaacuucgacaagaaucuuccaaacgagaaggugcuuccaaagcacucccugcuguacgaguacuucaccguguacaacgagcugaccaaggugaaguacgugacagagggcaugcggaagcccgccuuccucagcggugagcagaagaaggccaucguggaccugcuguucaagaccaaccggaaggugaccgugaagcagcugaaggaggacuacuucaagaagaucgagugcuucgacuccguggagaucuccggcguggaggaccgguucaacgccucccugggcaccuaccacgaccugcugaagaucaucaaggacaaggacuuccuggacaacgaggagaacgaggacauccuggaggacaucgugcugacccugacccuguucgaggacagggagaugauagaggagcggcugaagaccuacgcccaccuguucgacgacaaggugaugaagcagcugaagcggcggcgguacaccggcuggggccggcucagccggaagcugaucaaugggauccgagacaagcagagcggcaagaccauccuggacuuccugaaguccgacggcuucgccaaccggaacuucaugcagcugauccacgacgacucccugaccuucaaggaggacauccagaaggcccaggucagcggccagggcgacucccugcacgagcacaucgccaaucucgccgggucccccgccaucaagaaggggauccugcagaccgugaaggugguggacgagcuggugaaggugaugggccggcacaagccagagaacaucgugaucgagauggccagggagaaccagaccacucaaaaggggcagaagaacuccagggagcggaugaagcggaucgaggagggcaucaaggagcugggcucccagauccugaaggagcaccccguggagaacacucaacugcagaacgagaagcuguaccuguacuaccugcagaaugggcgagacauguacguggaccaggagcuggacaucaaccggcucagcgacuacgacguggaccacaucguuccccagagcuuccugaaggacgacagcaucgacaacaaggugcugacccgguccgacaagaaccggggcaaguccgacaacguucccucagaggaagucgugaagaagaugaagaacuacuggcggcagcugcugaacgccaagcugaucacucaacggaaguucgacaaucucaccaaggccgagcgggguggccucagcgagcuggacaaggccggguucaucaagcggcagcugguggagacccggcagaucaccaagcacguggcccagauccuggacucccggaugaacaccaaguacgacgagaacgacaagcugaucagggaagucaaggugaucacccugaaguccaagcuggucagcgacuuccggaaggacuuccaguucuacaaggugagggagaucaacaacuaccaccacgcccacgacgccuaccugaacgcugugguuggcaccgcacugaucaagaaguaccccaagcuggaguccgaguucguguacggcgacuacaagguguacgacgugcggaagaugauagccaaguccgagcaggagaucggcaaggccaccgccaaguacuucuucuacuccaacaucaugaacuucuucaagacagagaucacccuggccaauggugagauccggaagcggccccugaucgagaccaauggugagaccggugagaucgugugggacaaggggcgagacuucgccaccgugcggaaggugcucagcaugccccaggugaacaucgugaagaagacagaaguccagaccgguggcuucuccaaggagagcauccuuccaaagcggaacuccgacaagcugaucgcccgcaagaaggacugggaccccaagaaguacgguggcuucgacucccccaccguggccuacuccgugcuggugguggccaagguggagaaggggaaguccaagaagcugaaguccgugaaggagcugcugggcaucaccaucauggagcgguccuccuucgagaagaaccccaucgacuuccuggaagccaagggguacaaggaagucaagaaggaccugaucaucaagcuuccaaaguacucccuguucgagcuggagaaugggcggaagcggaugcuggccuccgccggugagcugcagaaggggaacgagcuggcacuucccucaaaguacgugaacuuccuguaccuggccucccacuacgagaagcugaagggguccccagaggacaacgagcagaagcagcuguucguggagcagcacaagcacuaccuggacgagaucaucgagcagaucuccgaguucuccaagcgggugauccuggccgacgccaaucucgacaaggugcucagcgccuacaacaagcaccgagacaagcccaucagggagcaggccgagaacaucauccaccuguucacccugaccaaucucggugcccccgcugccuucaaguacuucgacaccaccaucgaccggaagcgguacaccucgacuaaggaaguccuggacgccacccugauccaccagagcaucaccggccuguacgagacccggaucgaccucagccagcuggguggcgacggugguggcucccccaagaagaagcggaagguguagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacacccucgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaucuag

SEQ ID NO: 201 - RNA, 4573 nt, Artificial Sequence
  Synthetic: mRNA with SEQ ID No. 46 Cas9 ORF and 3′UTR mtRNR1-AES
gggaagcucagaauaaacgcucaacuuuggccggaucugccaccauggacaagaaguacagcaucggccuggacaucggcaccaacuccguuggcugggcugugaucaccgacgaguacaagguucccucaaagaaguucaaggugcugggcaacaccgaccggcacagcaucaagaagaaucucaucggugcacugcuguucgacuccggugagaccgccgaagccacccggcugaagcggaccgcccgccggcgguacacccggcggaagaaccggaucugcuaccugcaggagaucuucuccaacgagauggccaagguggacgacuccuucuuccaccggcuggaggaguccuuccugguggaggaggacaagaagcacgagcggcaccccaucuucggcaacaucguggacgaagucgccuaccacgagaaguaccccaccaucuaccaccugcggaagaagcugguggacucgacugacaaggccgaccugcggcugaucuaccuggcacuggcccacaugauaaaguuccggggccacuuccugaucgagggcgaccugaacccugacaacuccgacguggacaagcuguucauccagcuggugcagaccuacaaccagcuguucgaggagaaccccaucaacgccuccggcguggacgccaaggccauccucagcgcccgccucagcaagucccggcggcuggagaaucucaucgcccagcuuccaggugagaagaagaaugggcuguucggcaaucucaucgcacucagccugggccugacucccaacuucaaguccaacuucgaccuggccgaggacgccaagcugcagcucagcaaggacaccuacgacgacgaccuggacaaucuccuggcccagaucggcgaccaguacgccgaccuguuccuggcugccaagaaucucagcgacgccauccugcucagcgacauccugcgggugaacacagagaucaccaaggccccccucagcgccuccaugauaaagcgguacgacgagcaccaccaggaccugacccugcugaaggcacuggugcggcagcagcuuccagagaaguacaaggagaucuucuucgaccagagcaagaauggguacgccggguacaucgacgguggugccucccaggaggaguucuacaaguucaucaagcccauccuggagaagauggacggcacagaggagcugcuggugaagcugaacagggaggaccugcugcggaagcagcggaccuucgacaaugggagcaucccccaccagauccaccugggugagcugcacgccauccugcggcggcaggaggacuucuaccccuuccugaaggacaacagggagaagaucgagaagauccugaccuuccggauccccuacuacguuggcccccuggcccgcggcaacucccgguucgccuggaugacccggaaguccgaggagaccaucacucccuggaacuucgaggaagucguggacaagggugccuccgcccagagcuucaucgagcggaugaccaacuucgacaagaaucuuccaaacgagaaggugcuuccaaagcacucccugcuguacgaguacuucaccguguacaacgagcugaccaaggugaaguacgugacagagggcaugcggaagcccgccuuccucagcggugagcagaagaaggccaucguggaccugcuguucaagaccaaccggaaggugaccgugaagcagcugaaggaggacuacuucaagaagaucgagugcuucgacuccguggagaucuccggcguggaggaccgguucaacgccucccugggcaccuaccacgaccugcugaagaucaucaaggacaaggacuuccuggacaacgaggagaacgaggacauccuggaggacaucgugcugacccugacccuguucgaggacagggagaugauagaggagcggcugaagaccuacgcccaccuguucgacgacaaggugaugaagcagcugaagcggcggcgguacaccggcuggggccggcucagccggaagcugaucaaugggauccgagacaagcagagcggcaagaccauccuggacuuccugaaguccgacggcuucgccaaccggaacuucaugcagcugauccacgacgacucccugaccuucaaggaggacauccagaaggcccaggucagcggccagggcgacucccugcacgagcacaucgccaaucucgccgggucccccgccaucaagaaggggauccugcagaccgugaaggugguggacgagcuggugaaggugaugggccggcacaagccagagaacaucgugaucgagauggccagggagaaccagaccacucaaaaggggcagaagaacuccagggagcggaugaagcggaucgaggagggcaucaaggagcugggcucccagauccugaaggagcaccccguggagaacacucaacugcagaacgagaagcuguaccuguacuaccugcagaaugggcgagacauguacguggaccaggagcuggacaucaaccggcucagcgacuacgacguggaccacaucguuccccagagcuuccugaaggacgacagcaucgacaacaaggugcugacccgguccgacaagaaccggggcaaguccgacaacguucccucagaggaagucgugaagaagaugaagaacuacuggcggcagcugcugaacgccaagcugaucacucaacggaaguucgacaaucucaccaaggccgagcgggguggccucagcgagcuggacaaggccggguucaucaagcggcagcugguggagacccggcagaucaccaagcacguggcccagauccuggacucccggaugaacaccaaguacgacgagaacgacaagcugaucagggaagucaaggugaucacccugaaguccaagcuggucagcgacuuccggaaggacuuccaguucuacaaggugagggagaucaacaacuaccaccacgcccacgacgccuaccugaacgcugugguuggcaccgcacugaucaagaaguaccccaagcuggaguccgaguucguguacggcgacuacaagguguacgacgugcggaagaugauagccaaguccgagcaggagaucggcaaggccaccgccaaguacuucuucuacuccaacaucaugaacuucuucaagacagagaucacccuggccaauggugagauccggaagcggccccugaucgagaccaauggugagaccggugagaucgugugggacaaggggcgagacuucgccaccgugcggaaggugcucagcaugccccaggugaacaucgugaagaagacagaaguccagaccgguggcuucuccaaggagagcauccuuccaaagcggaacuccgacaagcugaucgcccgcaagaaggacugggaccccaagaaguacgguggcuucgacucccccaccguggccuacuccgugcuggugguggccaagguggagaaggggaaguccaagaagcugaaguccgugaaggagcugcugggcaucaccaucauggagcgguccuccuucgagaagaaccccaucgacuuccuggaagccaagggguacaaggaagucaagaaggaccugaucaucaagcuuccaaaguacucccuguucgagcuggagaaugggcggaagcggaugcuggccuccgccggugagcugcagaaggggaacgagcuggcacuucccucaaaguacgugaacuuccuguaccuggccucccacuacgagaagcugaagggguccccagaggacaacgagcagaagcagcuguucguggagcagcacaagcacuaccuggacgagaucaucgagcagaucuccgaguucuccaagcgggugauccuggccgacgccaaucucgacaaggugcucagcgccuacaacaagcaccgagacaagcccaucagggagcaggccgagaacaucauccaccuguucacccugaccaaucucggugcccccgcugccuucaaguacuucgacaccaccaucgaccggaagcgguacaccucgacuaaggaaguccuggacgccacccugauccaccagagcaucaccggccuguacgagacccggaucgaccucagccagcuggguggcgacggugguggcucccccaagaagaagcggaagguguagcaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacacccugguacugcaugcacgcaaugcuagcugccccuuucccguccuggguaccccgagucucccccgaccucgggucccagguaugcucccaccuccaccugccccacucaccaccucugcuaguuccagacaccucccucgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaucuag

SEQ ID NO: 202 - DNA, 278 bp, Artificial Sequence
  Synthetic: 3′ AES-mtRNR1 UTR
ctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacacc

SEQ ID NO: 203 - DNA, 278 bp, Artificial Sequence
  Synthetic: 3′ mtRNR1-AES UTR
caagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggtactgcatgcacgcaatgctagctgcccctttcccgtcctgggtaccccgagtctcccccgacctcgggtcccaggtatgctcccacctccacctgccccactcaccacctctgctagttccagacacctcc

SEQ ID NO: 204 - DNA, 260 bp, Artificial Sequence
  Synthetic: 3′ XBG-XBG UTR
accagcctcaagaacacccgaatggagtctctaagctacataataccaacttacactttacaaaatgttgtcccccaaaatgtagccattcgtatctgctcctaataaaaagaaagtttcttcacattctaccagcctcaagaacacccgaatggagtctctaagctacataataccaacttacactttacaaaatgttgtcccccaaaatgtagccattcgtatctgctcctaataaaaagaaagtttcttcacattct